(19) United States
(12) Patent Application Publication
(10) Pub. No.: US 2005/0176060 A1
(43) Pub. Date: Aug. 11, 2005

(54) Crystal structure

(76) Inventors: Neil McDonald, London (GB); Judith Murray-Rust, London (GB); James Mitchell Leiper, London (GB); Mark McAlister, London (GB); Patrick John Thompson Vallance, London (GB)

Correspondence Address: NIXON & VANDERHYE, PC, 1100 N GLEBE ROAD, 8TH FLOOR, ARLINGTON, VA, 22201-4714, US

(21) Appl. No.: 10/362,026

(22) PCT Filed: Aug. 17, 2001
(86) PCT No.: PCT/GB01/03687

(30) Foreign Application Priority Data
Aug. 18, 2000 (GB) 0020449.5

(51) Int. Cl. G01N 33/53; G06F 19/00; G01N 33/48; G01N 33/50

(52) U.S. Cl. 435/7.1; 702/19

(57) ABSTRACT

Structural coordinates obtainable by subjecting a crystal comprising a dimethylarginine dimethylaminohydrolase (DDAH) or a fragment thereof or an argininc deliminase (DI) or a fragment thercof to X-ray diffraction measurements and deducing the structural coordinales from the diffraction measurements arc used to identify, screen, characteristic, design or modify a chemical entity. The chemical entities so generated may be used in methods of Therapy or to identify the presence or absence of a DDAH or DI substrate.

### FIELD OF THE INVENTION

The invention relates to a crystal of an enzyme and a method for making that crystal. The structural coordinates of the crystal may be used to identify or design chemical entities which are modified enzymes or which bind to the enzyme.

### BACKGROUND OF THE INVENTION

N^{G}-Methylated derivatives of arginine within a wide range of eukaryotic proteins have been known for many years, although their function remains ill-defined. Methylation of arginine in proteins is carried out by enzymes of the protein-arginine methyl transferase PRMT) family. Proteolysis of methyl-arginine-containing proteins releases free methylarginine derivatives into the cytoplasm. Levels of methylarginines vary greatly between different tissues and elevated levels are found in a variety of pathological conditions and clinical disorders. Catabolism of two the asymmetrically methylated arginine derivatives N^{G},N^{G}-dimethyl-L-arginine (ADMA), and N^{G}-monomethyl-L-arginine (L-NMMA), to citrulline and mono- or di-methylamine is carried out by dimethylarginine dimethylaminohydrolase (DDAH, EC 3.5.3.18), which does not, however, hydrolyse symmetrical N^{G},N^{G′}-dimethyl-L-arginine (SDMA).

ADMA and L-NMMA (but not SDMA) are reversible inhibitors of all three isoforms of nitric oxide synthase (NOS) and may act as endogenous regulators of NOS in vivo. Thus DDAH, which controls levels of asymmetrically methylated arginine derivatives, may have therapeutic potential through its ability to indirectly influence the activity of NOS.

Two human DDAH isoforms have been identified—DDAH I and DDAH II and this is the case with some other mammals. Both enzymes have restricted tissue distributions which closely match either neuronal NOS or endothelial NOS respectively, suggesting an isoform-specific mechanism for regulating NOS activity via methylarginine. More recently microbial DDAH enzymes have been identified (Santa Maria et al., 1999) and so far each species has only one isoform. Their functional role is likely to be different from that in mammals, since bacteria lack NOS. Sequence comparisons have suggested a similarity between human DDAH and arginine deiminase (DI, EC 3.5.3.6), a microbial enzyme that converts arginine to citrulline and ammonia.

### SUMMARY OF THE INVENTION

We have determined the crystal structure of a bacterial DDAH from Pseudomonas auruginosa (PaDDAH).

According to the present invention there is thus provided use of the structural coordinates obtainable by subjecting by subjecting a crystal comprising a dimethylarginine dimethylaminohydrolase (DDAH) or a fragment thereof or an arginine deiminase (DI) or a fragment thereof to X-ray diffraction measurements and deducing the structural coordinates from the diffraction measurements, to identify, screen, characterise, design or modify a chemical entity.

The invention also provides:
a chemical entity generated by a use according to the invention;
a method for identifying, screening, characterising or designing a chemical entity which is a modified DDAH or DI or binds to a DDAH or DI, which method comprises comparing a structural model of the DDAH or DI with a structural model for said chemical entity, and thereby determining whether said chemical entity is likely to be a modified DDAH or DI or bind to the DDAH or DI, wherein said structural model of the DDAH is derived from structural coordinates determined by subjecting to X-Ray diffraction measurements a crystal comprising a DDAH or DI or a fragment thereof;
a chemical entity identified by a method of the invention;
a method for treating a host suffering from a condition in which abnormal nitric oxide metabolism is implicated or a bacterial infection, which method comprises administering to the host a therapeutically effective amount of a chemical entity of the invention;
a method for identifying the presence or absence of an asymmetricaliy methylated arginine derivative in a sample, which method comprises:
   (a) contacting the sample with a modified DDAH of the invention; and
   (b) determining whether the modified DDAH binds to an asymmetrically methylated arginine derivative;
a crystal comprising a DDAH or a fragment thereof or a DI or a fragment thereof;
a method for preparing a crystal, which method comprises forming a crystal of a substance comprising a DDAH or a DI;
a DDAH or DI which is an inactive mutant; and
a machine readable data storage medium comprising a data storage material encoded with machine readable data which when read by an appropriate machine is capable of displaying a three dimensional representation of a crystal of the invention.

### BRIEF DESCRIPTION OF THE FIGURES

FIG. 1 shows the overall fold of DDAH; every tenth residue is numbered.

FIG. 2 shows a worm representation of the backbone of DDAH with the catalytic residues indicated: Ser, (S, mutant from Cys); His (H) ; Glu (E) and the citrulline inhibitor (C).

### DETAILED DESCRIPTION OF THE INVENTION

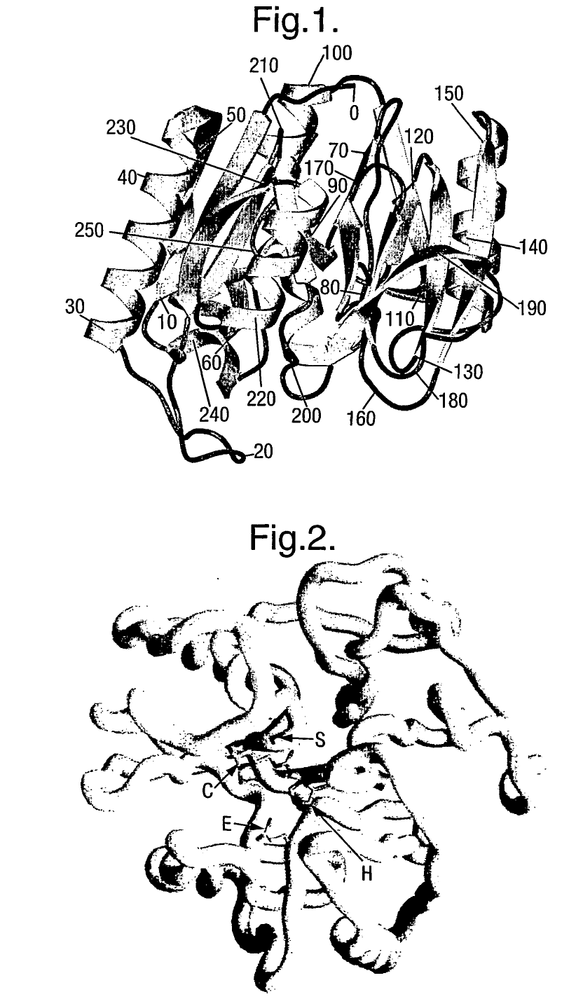

We have successfully crystallised a bacterial DDAH from Pseudomonas auruginosa (PaDDAH). This is the first crystallisation of a DDAH. This has allowed us to determine the crystal structure of PADDAH, revealing that it belongs to a structural superfamily of arginine-modifying enzymes which includes arginine:glycine amidinotransferase (AT, EC 2.1.4.1) and arginine deiminase (DI, EC 3.5.3.6). The three-dimensional structure of PADDAH is set out in FIG. 1. We have further identified Glu114, His162 and Cys249 as residues that are implicated in the active site. Site-directed mutagenesis of each of these residues produced enzymes that were inactive. A Cys:ser (C249S) mutant was used in the determination of crystal structures of a DDAH:substrate (ADMA) complex and a DDAH:product (citrulline) complex (FIG. 2). This allowed identification of ligand-binding residues around the active site.

Our crystallisation studies have allowed us to solve the structural coordinates of a DDAH enzyme. The invention provides use of the structure coordinates of an arginine modifying enzyme to identify, characterise, design or screen chemical entities. In particular, the chemical entities of interest are modified arginine-modifying enzymes or chemical entities which bind to an arginine-modifying enzyme. Particular arginine-modifying enzymes of interest are DDAHs and DIs.

Previously, it has not been possible to crystallise a DDAH or a DI. For example, attempts to crystallise mammalian DDAHs have not been successful and over-expression of DI in bacterial cells lead to cell death. We have now identified residues that when mutated result in an inactive PaDDAH. Those residues are conserved between PaDDAH and DI. It is therefore now possible to produce inactive DI and therefore to successfully over-express that enzyme. Thus, in the light of our results, crystallisation of DI is now possible for the first time. In addition, the similarities between bacterial and mammalian. DDAHs allow the use of our bacterial data in the design of chemical entities for use in mammalian contexts.

The chemical entities of the invention have uses in for example, therapy, diagnosis, quantification of DDAH or DI substrate concentrations and other investigative applications.

Typically, the structure coordinates used are obtainable by subjecting a crystal comprising a dimethylarginine dimethylaminohydrolase (DDAH) or a fragment thereof or an arginine deiminase (DI) to X-ray diffraction measurements and deducing the structural coordinates from the diffraction measurements, to identify, screen, characterise, design or modify a chemical entity.

The structural coordinates indicate the positions of individual atoms within the crystal give an indication of the available space for adjusting the position of individual atoms when designing a chemical entity.

The structure coordinates for native PADDAH are shown in Table II and those for C249S+citrulline are shown in Table III. Those structure coordinates are suitable for use according to the invention. In addition, structural coordinates obtainable by solving the data statistics in Table I may be used.

The crystal subjected to X-ray diffraction methods comprises a DDAH or a fragment thereof or a DI or a fragment thereof. The DDAH or DI may be from any source. Thus, the DDAH or DI may be a bacterial DDAH, for example one which originates from Pseudomonas aeruginosa, Strepyomyces coelicolor or Mycobacterium tuberculosis. Examples of bacterial arginine-modifying enzyme sequences are set out in Santa Maria et al., 1999, Mol. Microbiol. 33, 1278-1279.

Alternatively, a DDAH may be derived from a mammal, in which case the DDAH may be a DDAHI, for example human DDAHI or a DDAHII, for example human DDAHII. Suitable human DDAH sequences are set out in Leiper et al., 1999, Biochem. J. 343, 209-214.

A fragment of a DDAH or DI may also be used. Typically, a suitable fragment will be up to 10 amino acids in length, up to 20 amino acids in length, up to 50 amino acids in length, up to 100 amino acids in length, up to 200 amino acids in length, up to 300 amino acids in length, up to 400 amino acids in length, up to 500 amino acids in length or up to the entire length of the DDAH or DI polypeptide.

A preferred fragment is one which comprises all the amino acids required to form an active site of the DDAH or DI under study. Other preferred fragments are those which form antigenic epitopes of the DDAH or DI in question.

The DDAH may be a modified form of an entire DDAH or DI or a modified form of a fragment thereof. For example, the DDAH or DI may be modified by insertion, deletion, N-terminal or C-terminal addition, or substitution of an amino acid by another amino acid. Those types of modifications may be combined to produce a modified DDAH or DI for use in the invention. Amino acid substitutions may be conservative substitutions. From 1, 2, 3, 5 or 10 to 20, 30 or 50 modifications may be made in comparison to the corresponding wild-type DDAH or DI sequence. Thus, a DDAH or DI for use in the invention may be a mutant sequence. That is, it may have a polypeptide sequence which is different from that of the corresponding wild-type sequence. Typically, when crystallised, a DDAH or DI mutant or fragment thereof, will adopt a similar three-dimensional structure to that adopted by the corresponding DDAH or DI or a fragment thereof.

A mutant may be an inactive DDAH or DI. An inactive DDAH or DI is one which shows less than 20% of the enzyme activity shown by the corresponding wild-type enzyme, when the mutant and wild-type are compared using the calorimetric assay described in Leiper et al., 1999 (supra). Preferred, inactive enzymes are those which show less than 10% of the enzyme activity shown by the corresponding wild-type enzyme or preferably substantially no enzyme activity.

Preferred inactive DDAHs or DIs are those which carry a mutation at an amino acid equivalent to E114, H162 or C249 of PaDDAH. Those residues are directly involved in the reaction catalysed by PaDDAH. An equivalent amino acid is an amino acid in a DDAH derived from a species other Pseudomonas aeruginosa or a DI which occurs at an similar/corresponding position to a particular amino acid in the P. aeruginosa sequence and fulfils approximately the same function as the amino acid it is equivalent to, for example it takes part in catalysis. Examples of equivalent amino acids for a variety of arginine-modifying enzymes are set out in Table IV. For example, when compared to PADDAH, human DDAH shows conservation of the active site residues except that, in common with most species looked at, the catalytic Glu of PADDAH is Asp in the human sequence.

An equivalent amino acid will be readily identifiable by those skilled in the art, for example by carrying out sequence alignments. Computer programs for carrying out sequence alignments are will known to those skilled in the art and include, for example BLAST and PSI-BLAST as described in Altschul et al. (1997) Nucleic Acids Res. 25, 3389-3402.

A DDAH or DI for use in the invention may be chemically modified. Thus, particular amino acids can be labelled with a heavy metal, for example methionine residues may be labelled with selenium. A DDAH of DI for use in the invention may also be post-translationally modified. For example it may be glycosylated or comprise modified amino acid residues. A DDAH of DI can be in a variety of forms of polypeptide derivatives, including amides and conjugates with polypeptides.

Chemically modified DDAHs or DIs also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

A DDAH or DI may be isolated by any suitable means for use in crystallization studies. For example, a DDAH or a DI may be purified using biochemical means from a suitable source. Typically, however, it will be convenient to over-express a DDAH or a DI in cells and purify the DDAH or DI from those cells. Thus, a polynucleotide encoding a DDAH or a DI as described herein may be used in the construction of a vector. It may be necessary to use a polynucleotide encoding an inactive DDAH or DI as defined above. This may be necessary if over-expression of an active DDAH or DI disrupts cell function to the extent that expression cannot occur to a desired level.

Preferably, a polynucleotide in a vector is operably linked to a control sequence which is capable of providing for the expression of the coding sequence by a host cell when the construct in transferred into that cell, i.e. the vector is an expression vector. The term “operably linked” refers to ajuxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used to transfect or transform a host cell, for example, a bacterial, yeast, insect or mammalian host cell.

Promoters and other expression regulation signals may be selected to be compatible with the host cell for which expression is designed. For example, yeast promoters include S. cerevisiae GAL4 and ADH promoters, S. pombe nmtl and adh promoter. Mammalian include promoters such as P-actin promoters or the metallothionein promoter which can be induced in response to heavy metals such as cadmium. Viral promoters such as the SV40 large T antigen promoter or adenovirus promoters may also be used. All these promoters are readily available in the art. The vector may further include sequences flanking the polynucleotide giving rise to polynucleotides which help in the expression of the DDAH or DI.

Constructs which contain a polynucleotide encoding a DDAH or a DI may be transferred, for example by transformation or transfection, into a cell so that the cells express the DDAH or DI. Such cells may express the DDAH or DI transiently or stably. Suitable cells may be higher eukaryotic cell lines, such as mammalian cells or insect cells, lower eukaryotic cells, such as yeast or prokaryotic cells such as bacterial cells. The cell line may be selected so that appropriate post-translational modification occurs. It is also possible for the proteins of the invention to be transiently expressed in a cell line or on a membrane, such as for example in a baculovirus expression system. Such systems, which are adapted to express the proteins according to the invention, are also included within the scope of the present invention.

The DDAH or DI may be crystallised according to any method known to those skilled in the art.

X-ray diffraction may be carried out according to any method known to those skilled in the art. The data collected from X-ray diffraction experiments may be processed to deduce the structural coordinates of the DDAH or DI being studied using methods well known to those skilled in the art.

The invention also provides the use of the structural coordinates obtainable by subjecting a crystal comprising a DDAH or a fragment thereof or a DI or a fragment thereof bound to a substrate or product to X-ray diffraction measurements and deducing the structural coordinates from the diffraction measurements. Suitable substrates are asymmetric methylated arginine derivatives, for example N^{G},N^{G}-dimethyl-L-argine (ADMA) or N^{G}-monomethyl-L-arginine (L-NMMA) in the case of DDAH and arginine in the case of DI. A suitable product is citrulline for DDAH or DI.

When a crystals comprising an arginine-modifying enzyme bound to a substrate or product is used, typically an inactive form of a DDAH or DI is used. The DDAH or DI is inactive in the sense that it is binds a substrate but substantially cannot metabolize that substrate. An alternative suitable inactive DDAH or DI can typically be capable of binding the product without metabolizing that product. A suitable inactive DDAH or DI may be capable of binding both substrate and product.

The invention provides the use of structural coordinates to identify, characterise, design or screen a chemical entity. The chemical entity may be for example a modified DDAH or DI or a chemical entity which binds to a DDAH or a DI. A chemical entity which binds to a DDAH or a DI may be, for example, an inhibitor or an activator of that DDAH or DI.

A modified DDAH or DI may have a different sequence to its corresponding DDAH or DI. Alternatively, it may have the same sequence as its corresponding wild-type sequence, but comprise one or more chemically modified amino acids.

Preferred modifications are those which alter the activity characteristics of the DDAH or DI, but which do not substantially alter the shape of the DDAH or DI.

A chemical entity which binds to a DDAH or a DI is any chemical entity capable of forming an association with the DDAH or DI. The chemical entity may bind to a DDAH or DI non-specifically, in which case it will bind to other partners, or may bind specifically to a DDAH or a DI. Chemical entities which bind DDAH or DI may be small molecules, for example small organic or inorganic molecules. Alternatively, the chemical entity could be a large/macromolecule, for example a polypeptide or peptide. The polypeptide or peptide may be for example, an antibody or a fragment of an antibody.

An inhibitor of a DDAH or a DI is one which, when present, produces a measurable reduction in DDAH or DI activity in the calorimetric assay described in Leiper et al., 1999 (supra). An activator of a DDAH or DI is one which, when present, produces a measurable increase in DDAH or DI activity in the colorimetric assay described in Leiper et al., 1999 (supra).

Preferred inhibitors are those which reduces DDAH or DI activity by at least 10%, at least 20%, at least 30%, at least 40% at least 50%, at least 60%, at least 70%, at least 80%, at least 90%, at least 95% or at least 99% at a concentration of the inhibitor of 1 μg ml⁻¹, 10 μg ml⁻¹, 100 μg ml⁻¹, 500 μg ml⁻¹, 1 mg ml⁻¹, 10 mg ml⁻¹, 100 mg ml⁻¹.

Preferred activators are those which increase DDAH or DI activity by at least 10%, at least 25%, at least 50%, at least 100%, at least, 200%, at least 500% or at least 1000% at a concentration of the activator 1 μg ml⁻¹, 10 μg ml⁻¹, 100 μg ml⁻¹, 500 μg ml⁻¹, 1 mg ml⁻¹, 10 mg ml⁻¹, 100 mg m⁻¹.

The percentage inhibition or activation represents the percentage decrease or increase in activity in a comparison of assays in the presence and absence of the test substance. Any combination of the above mentioned degrees of percentage inhibition or activation and concentration of inhibitor or activator may be used to define an inhibitor or activator of the invention, with greater inhibition or activation at lower concentrations being preferred.

Inhibition may occur if, for example, the inhibitor resembles the substrate and binds at the active site of the DDAH or DI. The substrate is thus prevented from binding to the same active site and the rate of catalysis is reduced by reducing the proportion of enzyme molecules bound to substrate (competitive inhibition): An inhibitor may also exert its effects by non-competitive inhibition where the inhibitor and substrate can bind simultaneously to the DDAH or DI and thus bind at different non-overlapping sites. Inhibition occurs as the turnover number of the DDAH of DI decreases.

Activation may occur, for example, if the modulator increases the affinity of the substrate for the DDAH or DI or vice versa. This means that the proportion of DDAH or DI molecules bound to a substrate is increased and the rate of catalysis will thus increase.

The structural coordinates of a DDAH of a DI crystal, in particular a crystal of a DDAH of a DI bound to its substrate, may allow the skilled person to predict which amino acids may be important in active site formation and-which amino acids are important in contacting the substrate and product. The substrate binding site bound to the DDAH or DI peptide to be shown as a two dimensional representation, for example as a LIGPLOT or a three dimensional representation by physical models or as displayed on a computer screen. Such representations can be used to design modifications of a DDAH or DI, for example in the design of a DDAH or DI with altered activity characteristics, to design chemical entities which bind a DDAH or a DI or to design modifications to chemical entities which bind a DDAH or a DI.

Example of modifications include modification to increase the avidity of a DDAH or a DI for its substrate or product. That type of modification may increase the activity of a DDAH or a DI, increase the avidity of an inactive DDAH or DI for a DDAH or DI substrate or may alter the substrate specificity of the DDAH or DI in question. Alternatively, modifications may increase the avidity with which a chemical entity binds DDAH or DI. Thus modifications may be made to a known inhibitor or activator of DDAH or DI so that they bind DDAH or DI with increased affinity, thus increasing their efficacy.

The avidity of a DDAH or DI for its substrate or product or of a chemical entity for a DDAH of DI may be increased by modifying the active site to increase the amount and number of interactions favourable to binding. Favourable interactions may be increased by extending the structure of the substrate or product binding site or the chemical entity into spaces which are shown in the two dimensional or three dimensional representations to be unoccupied or filled with water molecules.

The representations of the structures may be used in other ways to modify the structure of a DDAH or DI. The representations of a DDAH or DI active site may be used to model constraints by the putative introduction of covalent bonds between the atoms which come close together when a DDAH or DI binds to a substrate or product; one or more chemical linkers may be used between atoms of a DDAH or DI to constrin the active site to the required conformation, and/or one or more amino acids of a DDAH or DI may be replaced by analogues of the natural amino acids which help to constrain the conformation of the active site. On the basis of such modelling, modified DDAHs or DIs may be identified, characterised, designed or screened.

Representation of the active site bound to a substrate may be used to predict which residues of a DDAH or DI are likely to be involved in the steric hindrance. Such residues may be modified, replaced or deleted to decrease the steric hindrance in order to increase avidity.

In general it will be necessary to process the structural coordinates obtainable according to the invention in computer-based methods in order to identify or design chemical identities with a desired molecular structure or to identify chemical entities whose structure is similar to all or a part of another chemical entity of interest.

Thus, chemical entities which have a structure similar to a DDAH or a fragment thereof or a DI or a fragment thereof may be identified or designed. Also, chemical entities which bind to a DDAH or a DI may be identified or designed. Preferably, that type of chemical entity will bind at the active site of a DDAH or DI.

Such computer-based methods fall into two-broad classes: database methods and de novo design methods. In database methods the chemical entity of interest is compared to all chemical entities present in a database of chemical structures and chemical identities whose structure is in some way similar to the compound of interest are identified. The structures in the database are based either on experimental data, generated by NMR or x-ray crystallography, or modeled three-dimensional structures based on two-dimensional data. In de novo design methods, models of chemical entities whose structure is in some way similar to the compound of interest are generated by a computer program using information derived from known structures and/or theoretical rules.

Using the structural coordinates, a three dimensional representation of the surface of a DDAH or a DI, in particular the active site, can be produced using Catalyst Software such as Catalyst/SHAPE, Catalyst/COMPARE, DBServer HipHop Ludi, MCSS and Hook which are available from Molecular Simulations Ltd., 240/250 The Quorum, Barnwell Road, Cambridge, England. Modified DDAHs or DIs, for example can be produced either by computationally identifying compounds which have a similar surface to the DDAH, or by computationally designing compounds with surfaces which are likely to bind a substrate or product.

Various methods can be used to produce a three dimensional surface which is the same or similar to the surface of a chemical entity which will bind DDAH or DI, preferably at the active site. Based on this shape, packages such as Catalyst/SHAPE and Catalyst/COMPARE can be used to select compounds from databases which have a similar three dimensional shape. Thus chemical entities which bind a DDAH or DI, for example, can be produced either by computationally identifying compounds which have a similar surface, or by computationally designing compounds with surfaces which are likely to bind to a DDAH or a DI.

The success of both database and de novo methods for identifying compounds with activities similar to the compound of interest depends on the identification of the functionally relevant portion of the compound of interest. For a chemical entity that interacts with a DDAH or DI, the functionally relevant portion is referred to as a pharmacophore. A pharmacophore is an arrangement of structural features and functional groups important for biological activity. Similarly, one can identify one or more pharmacophores for a given chemical entity which binds to a DDAH or a DI. In this case, the pharmacophore is a group of atoms that play an important role in binding to a DDAH or DI and thus for activation or inhibition of a DDAH of DI, for example.

The data provided herein, concerning the structures of a DDAH and the structure of a DDAH bound to a substrate permits the identification of pharmacophores important for binding to arginine-modifying enzymes, in particular to a DDAH or a DI.

Programs suitable for pharmacophore selection and design include DISCO (Abbott Laboratories, Abbott Park, Ill.) and catalyst (Bio-CAD Corp., Mountain view, Calif.). Databases of chemical structures are available from Cambridge Crystallographic Data Centre (Cambridge, UK) and Chemical Abstracts Service (Columbus, Ohio). De novo design programs include Ludi (Biosym Technologies Inc., San Diego, Calif.) and Aladdin (Daylight Chemical Information Systems, Irvine, Calif.). Such programs are well known to those skilled in the art. Packages such as DBServer1 and HipHop can be used to search databases for compounds whose surfaces are described by similar pharmacophores.

Once a chemical entity has been designed or selected by the above methods, the efficiency with which that entity binds a DDAH or a DI, a substrate or a product may be tested and optimized using computational or experimental evaluation. Various parameters can be optimized depending on the desired result. These include, but are not limited to, specificity, avidity, on/off rates, and other characteristics readily identifiable by those skilled in the art.

The computational means may employ packages such as Catalyst/SHAPE, Catalyst/COMPARE, DBServer, HipHop, Ludi and MCSS to evaluate selected modified DDAHs or DIs and new DDAH or DI binding entities. The experimental means may comprise, for example, the calorimetric assay described in Leiper et al., 1999 (supra).

Thus, one may optionally make substitutions, deletions, or insertions in some of the components of the DDAH or DI or to the DDAH or DI binding entity in order to improve or modify the binding properties. Generally, initial substitutions are conservative, i.e. the replacement group will have approximately the same size, shape, hydrophobicity and charge as the original component. Such modifications can be computationally or experimentally evaluated in the same manner as the first candidate chemical entities. If necessary, further modifications can be made. This process of evaluating and modifying may be iterated any number of times.

Thus, the invention provide a method for identifying, screening, characterising or designing a chemical entity which is a modified DDAH or DI or binds to a DDAH or DI, which method comprises comparing a structural model of the DDAH or DI with a structural model for said chemical entity, and thereby determining whether said chemical entity is likely to bind to or mimic the DDAH or DI, wherein said structural model of the DDAH or DI is derived from structural coordinates determined by subjecting to X-Ray diffraction measurements a crystal comprising a DDAH or a DI. The invention also provides a chemical entity identified by a method of the invention for identifying, screening, characterising or designing a chemical entity.

A chemical entity identified in the invention, for example by a use according to the invention or by a method of the invention for identifying, screening, characterizing or designing a chemical entity which is a modified DDAH or a modified DI or binds to a DDAH or DI, may be used in the treatment of medical or veterinary conditions. Thus, the invention provides chemical entities for use in a method of treatment of the human or animal body by therapy. In particular, they may be used in a method of treatment of a condition in which the abnormal metabolism of NO is implicated. A chemical entity identified in the invention may also be used for the manufacture of a medicament for use in the treatment of a condition in which the abnormal metabolism of NO is implicated. The condition of a patient suffering from that type of condition can be improved by administration of a chemical entity identified in the invention. A therapeutically effective amount of a chemical entity identified in the invention may be given to a human patient in need thereof.

A chemical entity which is an activator of a DDAH may be used in the treatment of conditions in which reduced NO production is implicated. In particular such conditions as hyperlipidaemia, renal failure, hypertension, restenosis after angioplasty, complications of heart failure, or atherosclerosis and its complications may be treated and patients with schizophrenia, multiple sclerosis or cancer may also be treated. A modified DDAH which shows increased DDAH activity in comparison to its corresponding wild-type DDAH may also be used in the treatment of the above conditions.

A chemical entity which is an inhibitor of a DDAH may be used in the treatment of conditions in which increased NO production is implicated. In particular, conditions such as ischeamia-reperfusion injury of the brain or heart, cancer, lethal hypotension in severe inflammatory conditions such as septic shock or multi-organ failure, or local and systemic inflammatory disorders including arthritis, skin disorders, inflammatory cardiac disease or migraine may be treated.

Alternatively, a chemical entity which is an inhibitor of a DDAH could be used as a joint therapy together with an inhibitor of NOS activity (for example, a methylarginine). For example, a specific inhibitor of a DDAH isoform could be used with the methylarginine L-NMMA. This approach may radically alter the activity profile of L-NMMA and may result in L-NMMA having an increased inhibitory effect for a specific NOS isoform. Thus, the invention provides products containing an inhibitor of a DDAH activity and/or expression and a methylarginine as a combined preparation for simultaneous, separate or sequential use in the treatment of ischeamia-reperfusion injury of the brain or heart, cancer, lethal hypotension in severe inflammatory conditions such as septic shock or multi-organ failure, or local and systemic inflammatory disorders including arthritis, slin disorders, inflammatory cardiac disease or migrane.

A chemical entity which is an inhibitor of a DDAH or a DI may also be used as an antimicrobial agent, for example an antibacterial agent. Therefore, the invention also provides a chemical entity for use in the treatment of a bacterial infection.

The formulation of a chemical entity of the invention will depend upon factors such as the nature of the exact antagonist, whether a pharmaceutical or veterinary use is intended, etc. A product of the invention may be formulated for simultaneous, separate or sequential use.

A product of the invention is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidnocaine hydrochloride.

Solutions for intravenous administration or infusion may contain as carrier, for example, sterile water or preferably they may be in the form of sterile, aqueous, isotonic saline solutions.

A therapeutically effective amount of product of the invention is administered to a patient. The does of a product of the invention may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated, the type and severity of the degeneration and the frequency and route of administration. Preferably, daily dosage levels are from 5 mg to 2 g.

The invention allows for the identification, screening, characterisation, design or modification of chemical entities which effect specific regulation a particular isoform DDAH and thus of NOS. Chemical entities which have effects specific for one particular DDAH isoform, for example a DDAHI or a DDAHIH, may be administered non-specifically as they will only modulate the expression or activity of a particular methylarginase and thus the activity of one particular isoform of NOS. Chemical entities which are peptides or polypeptides may be administered in the form of a naked nucleic acid construct. Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™).

Typically, nucleic acid constructs are mixed with the transfection agent to produce a composition. Preferably the naked nucleic acid construct, viral vector comprising the polynucleotide or composition is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, or transdermal administration.

The pharmaceutical composition is administered in such a way that the polynucleotide of the invention, viral vector for gene therapy, can be incorporated into cells at an appropriate area. When the polynucleotide of the invention is delivered to cells by a-viral vector, the amount of virus administered is in the range of from 10⁶ to 10¹⁰ pfu, preferably from 10⁷ to 10⁹ pfu, more preferably about 10⁸ pfu for adenoviral vectors. When injected, typically 1-2 ml of virus in a pharmaceutically acceptable suitable carrier or diluent is administered. When the polynucleotide of the invention is administered as a naked nucleic acid, the amount of nucleic acid administered is typically in the range of from 1 μg to 10 mg.

Where the polynucleotide giving rise to the product is under the control of an inducible promoter, it may only be necessary to induce gene expression for the duration of the treatment. Once the condition has been treated, the inducer is removed and expression of the polypeptide of the invention ceases. This will clearly have clinical advantages. Such a system may, for example, involve administering the antibiotic tetracycline, to activate gene expression via its effect on the tet repressor/VP16 fusion protein.

The use of tissue-specific promoters will be of assistance in the treatment of disease using the olypeptides, polynucleotide and vectors of the invention. It will be advantageous to be able express therapeutic genes in only the relevant affected cell types, especially where such genes are toxic when expressed in other cell types.

The routes of administration and dosages described above are intended only as a guide since a skilled physician will be able to determine readily the optimum route of administration and dosage for any particular patient and condition.

Chemical entities identified in the invention may be used in a method of diagnosis carried out on the human or animal body. Thus, a modified DDAH, for example an inactive modified DDAH, can be used to assay for the concentration of asymmetric methylarginines, for example ADMA, in a sample obtained from a subject. This may provide an indication of the status of a subject with respect to a particular medical condition, for example it may indicate whether or not the subject is suffering from a cardiovascular disease. Suitable samples include any body fluid, for example blood, urine or saliva. In addition, tissue samples may be used, for example a blood vessel biopsy. It will be apparent to those skilled in the art how such assays may be carried out. A modified DDAH, for example an inactive modified DDAH, may also be used for in vivo asymmetric methylarginine imaging. Generally, the modified DDAH is be labelled in such an application.

Chemical entities identified in the invention may also be used in a method for identifying the presence or absence of an asymmetrically methylated arginine derivative, for example ADMA, in a sample. The method comprises contacting the sample with a modified DDAH and determining whether the modified DDAH binds to an asymmetrically methylated arginine derivative. The method could be applied in the context of a radioimmunoassay or a ligand binding assay. Suitableassay formats are well known to those skilled in the art. The modified DDAH is preferably an inactive modified DDAH.

Similar methods may be carried out with a modified DI in order to determine the presence or absence of arginine in a sample.

The invention also provides a crystal comprising a DDAH or a fragment thereof or a DI or a fragment thereof. The DDAH or DI may be any DDAH or DI, for example one which is described above. The DDAH or DI may be crystallised in combination with its substrate or its product. The crystal may have the data statistics set out in Table I. Alternatively or in addition, the crystal may have the structural coordinates set out in Table II.

The invention also provides a DDAH or a DI. A DDAH or a DI of the invention is preferably one which has been identified, designed and/or modified by use of the structural coordinates obtained by subjecting a crystal of the invention to X-ray diffraction. Preferred DDAHs or DIs are those which have been inactivated. Examples of such DDAHs or Dis are those in which an amino acid equivalent to E114, H162 or C249 in the PaDDAH is replaced by a different amino acid. The term “equivalent” in this context is explained above. Preferably, the different amino acid is alanine or a sterically similar residue.

Other residues which form part of the active pocket and stabilise its structure include those equivalent to D60, F63, E65, D66, R85, E88, D117, R132, L161, I243, K164, S248, S251, L252 and R253 in the PaDDAH sequence. Thus, the invention provides a DDAH which is mutated, for example substituted, at one or more residues corresponding to one of the above list. Two types of mutation are favoured which may provide inactive DDAHs or DIs: (i) substitution of a residue which hydrogen bonds directly to the substrate, such those equivalent to D66 and E65 in the PaDDAH sequence, with a basic residue; and (ii) substitution of hydrophobic residues, such as those equivalent to F63 and L161 which contact the hydrophobic part of the ligand either to block the active site or to provide less favourable interactions. More extensive mutation, for example changing the length of the flexible loops surrounding the active site cleft could also be used to affect activity of a DDAH or a DI. A DDAH or DI of the invention may be used to prepare a crystal of the invention which is then subjected to X-ray diffraction measurements according to a use of the invention.

The invention further provides a polynucleotide which encodes a DDAH or a DI of the invention and a vector which contains that polynucleotide. A polynucleotide of the invention can be used to prepare a construct wherein the polynucleotide is operably linked to a promoter. Such a construct can then be used to transform or transfect a cell. Such cells may be used to over-express a DDAH or a DI of the invention. The DDAH or DI so-expressed may be used to prepare a crystal and that crystal used according to the invention to identify, screen, characterise, design or modify a chemical entity. Suitable promoters and cells are described above.

The invention also provides a method for preparing a crystal of the invention. The method comprises crystallizing a substance comprising a DDAH or a fragment thereof or a DI or a fragment thereof. The DDAH or DI may be a DDAH or a DI of the invention.

The following Example illustrates the invention:

### EXAMPLE

Materials and Methods

Preparation of P.a DDAH.

Samples of wild-type PaDDAH and the C249S mutant form were produced for crystallisation as described below. BL21(DE3) cells (Novagen Inc.) transformed with pPROX.Hta.PaDDAH or pPROX.Hta.PaDDAH-C249S were grown at 30° C. in Luria-Bertani medium with carbenicillin (100 μg/ml) to an optimal density of 0.8 at 600 nm, before induction with 0.5 mM IPTG. Cultures were harvested by centrifugation after 4-6 hrs. For biosynthetic-labelling of PaDDAH with seleno-methionine, B834(DE3) cells transformed with pPROX.Hta.DDAH were grown at 30° C. in minimal medium supplemented with selenomethionine, and induced as described above and harvested after 12 hrs. Cell-pellets were resuspended in 25 ml of buffer A (50 mM NaH₂PO₄, 10 mM imidazaole, 0.3 M NaCl, 5 mM b-mercaptoethanol, 1 mM PMSF, 5 mM benzamidine, adjusted to pH 8.0 with NaOH) and frozen at −80° C. DNAaseI was added to the thawed cell suspension to 10 μg/ml, before disrupting the cells by French-press (Spectronic Instruments Inc.). The cell-lysate was centrifuged at 40,000 g for 30 mins. at 4° C. The lysate supematant was purified on a Ni—NTA (Qiagen Inc.) column (Vₜ=10 ml, dᵢ=16 mm), by washing in 100 ml of buffer A and then 100 ml of buffer B (as buffer A, but with 50 mM imadazole pH 8.0); before step-elution with 50 ml of buffer C (as buffer A, but with 250 mM imidazole). EDTA (10 mM) was added to the DDAH pool, before further purification by gel-filtration on a Superdex 200 column (Vₜ=100 ml, dᵢ=16 mm) equilibrated in 20 mM Tris pH 8.0, 100 mM NaCl, 10 mM DTT. The purified DDAH pool was then digested with rTEV protease (Gibco-BRL) at room temperature overnight, using 500 U rTEV per 10 mg PaDDAH. The cleaved 6 His-tag and rTEV were removed by batch adsorption onto 2 ml of Ni—NTA agarose at 4° C. PaDDAH was then re-purified by gel filtration. The de-tagged PaDDAH pool was then buffer-exchanged into 25 mM Tris, 10 mM DTT pH 8.0 using a Vivaspin centrifugal concentrator (Sartorius) then concentrated to 14 mg/ml. A final yield of 10-15 mg of PaDDAH per litre of original culture was obtained. Protein concentrations of purified PaDDAH samples were estimated by absorbance measurements at 280 nm. The extinction coefficient of PaDDAH was calculated to be 12,960 M⁻¹ cm⁻¹ using the program “Peptidesort” (GCG Inc.).

Bio-Physical Analysis.

Mass spectra were collected on a VG Platform Electrospray Mass Spectrometer (Micromass). Samples were desalted by ultrafiltration into de-ionised water. Samples were then injected (10 μl) under standard conditions (80-200 pmol/μl in 50% acetonitrile, 0.25% fornic acid). Delivery solvent (50% acetonitrile) was pumped at a flow rate of 10 μl/min. Twelve 10 second scans were accumulated for each-sample over the m/z range 750-1150. Source temperature was set at 45° C. and the cone voltage was 41 volts. Spectra were processed using the ‘Masslynx’ software supplied with the instrument (version 3.0 b5). Horse heart myoglobin was used to calibrate the instrument (Mᵣ=16951.5 as determined by (Ashton et al.,1994). The masses of “de-tagged” wild-type and C249S mutant PaDDAH constructs were calculated to be 29,217 and 29,201 respectively. The observed masses matched the calculated masses to within experimental error (4 mass units) and indicated that both samples were essentially homogeneous.

Analytical gel-filtration and dynamic light scattering were used to estimate the apparent molecular weight and oligomeric state of PaDDAH over a range of protein concentrations. Both analyses suggested that DDAH was dimeric in solution over the concentration range of 0.1-10 mg/ml.

Analytical gel-filtration chromatography was performed using a Superdex 200 HR column (10/30) (Amersham-Pharmacia Biotech), equilibrated in 25 mM Tris, 100 mM NaCl, pH 7.0, 5 mM DTT at a flow-rate of 0.5 ml/min, using 100 μl sample volumes at 0.1, 1.0 and 10 mg/ml protein concentration. The column was calibrated with a range of protein molecular weight standards to allow estimation of apparent molecular mass (Preneta, 1990).

Dynamic light scattering measurements were obtained using a DynaPro-801 instrument equipped with a 100 μl cell (Protein Solutions, Charlottesville, Va.). Samples of PaDDAH, at concentrations of 0.5, 2.0, 6.0 and 12 mg/ml in 25 MM Tris, 100 mM NaCl, pH 7.0, 5 mM DTT buffer, were passed through 0.2 μm membrane filters (Whatman, UK) during injection. Measurements were taken at room temperature according to the manufacturer's instructions, and data was analysed using ‘Autopro’ software. The program allowed calculation of the translational diffusion coefficient, D, which was converted into apparent Stokes radii, Rsₐₚₚ via the Stokes-Einstein equation:
Rs,app=kT/6πη₀D
where k is the Boltzrnan constant, T the temperature in degrees Kelvin and η₀ the solvent viscosity that was assumed to be that of water. The instrument was calibrated with a set of twenty-three standard proteins ranging in size from 8-1250 k in Mᵣ, allowing reliable estimation of molecular mass from measurements of diffusion coefficients. The photon count-rate varied by less than 3% in all experiments. Sum of squares errors for all readings were less than 1.0 indicating that the data was of high quality with a high signal to noise ratio.

Analytical gel-filtration and dynamic light scattering were used to estimate the apparent molecular weight and oligomeric state of PaDDAH over a range of protein concentrations. Both analyses suggested that DDAH was dimeric in solution over the concentration range of 0.1-10 mg/mil. There was some evidence of a reduction in apparent molecular weight as the protein concentration was decreased suggesting some degree of dissociation of dimer. In addition dynamic light scattering data indicated that PaDDAH existed as an essentially mono-dispersed dimer at a protein concentration of 12 mg/ml.

Site-Directed Mutagenesis

The open reading frame of Pseudomonas DDAH was cloned in frame downstream of the polyhistidine and linker sequences of the plasmid pProEX HTa (Life Technologies). Recombinant plasmids were transfected into competent E. coli DH5α which were seeded into liquid cultures. When cultures had reached an OD of 0.5 protein expression was induced by the addition of IPTG (final conc 1 mM). Two hours post induction cells were harvested by centrifugation and lysed by sonication. Cytosolic protein was separated from cell debris by centrifugation and recombinant protein was purified by affinity chromatography (Nickel agarose binding to polyhistidine tag) followed by size fractionation. The polyhistidine and linker sequences were removed by proteolysis with recombinant tobacco envelope virus protease (Life technologies).

Site-directed mutagenesis was performed using a PCR based method. Complimentary oligonucleotides (forward and reverse) encoding the desired nucleotide substitution were designed. In separate PCR reactions, using cloned PaDDAH as the template, each of the mutagentic oligonucleotide was included with a primer complimentary to flanking vector sequences. The products of the two PCR reactions were gel purified, combined and the entire open reading frame was then amplified using the two vector primers previously included in the initial PCR reactions. PCR products were cloned into pProEX HTa and sequenced to verify the correct sequence.

Activity of the mutants was determined by the colorimetric assay described in Leiper et al. (1999), Biochem. J. 343, 209-214.

Crystallography

Crystallization. All crystals were grown at room temperature by the sitting drop method using 1.5 μl of each of the protein solution and the well solution per drop.

For the native and the SeMet derivatives the protein stock solution was between12 and 14 mg/ml in 50 mM Tris buffer, pH 8 with 5 mM DTT. For the co-crystallisation of mutant protein, ligand at a concentration giving 10-15:1 molar ratio was added to the protein solution. The well solutions were in the range 0.1M Tris pH8.5, 0.15-0.3M Na acetate, 25-35% w/v PEG4000.

Data Collection. Experimental details and data statistics for native, SeMet, citrulline- and ADMA-cocrystallised datasets are given in Table I. Data processing was with MOSFLM (Leslie, 1992, Joint CCP4 and ESF-EAMCB Newsletter on Protein Crystallography, No 26) and subsequent calculations used the CCP4 programs (CCP4, 1994, Collaborative Computing Project, Number 4. The CCP4 suite: programs for computational crystallography. Acta Crystallographica D, 50, 760-763.).

Structure solution and refinement. The structure of the SeMet derivative of PADDAH was solved by using SOLVE (Terwiliger and Berendzen, 1999, Automated structure solution for MIR and MAD. Acta Crystallographica D55, 849-861) using the MAD dataset. This revealed 10 out of a possible 14 Se sites, which were grouped in two sets of five related by a two-fold axis. Use of density modification and two-fold averaging in the program DM (Cowtan, 1994) produced a map in which most of the mainchain could be traced, with the help of the Se sites as landmarks. Native PaDDAH and the SeMet derivative crystallise in the same space group, but do not scale well together. The structure of native PaDDAH was solved by using the phases obtained from SOLVE for the SeMet derivative with the native F's as input to density modification and two-fold averaging-in DM. This showed a dimeric structure essentially the same as that of SeMet derivative, but with a small displacement of one molecule of the dimer. The same reflection set was used for cross-validation (R_{free}) calculation in both the native and SeMet datasets. Co-crystallisations of C249S PaDDAH with citrulline and ADMA each produced crystal of different habit from the native, and with higher resolution diffraction. This is reflected in the overall B values from a Wilson plot of 16 and 18 Å² respectively, compared with 40 Å² for both the native and SeMet datasets.

The citrulline- and ADMA-containing C249S mutant crystals were in space group C2 (1 molecule/asymmetric unit) and P21 (4 molecules/asymmetric unit) respectively, and they were each solved by molecular replacement using a partly refined native structure in AmoRe (Navaza, 1994, AmoRe: an Automated package for molecular replacement. Acta Crystallographica A, 50, 157-163.). The cell dimensions of the ADMA-containing crystals show similarity to that of the native, while the cell for the citrulline derivative is not obviously related.

Model building was done with the program O (Jones et al., 1991, Improved methods for building protein models in electron density maps and the location of errors in these models. Acta Cryst. A, 47, 110-119.) and refinement with refmac (Murshodov, et al., 1997, Refinement of Macromolecular Structures by the Maximum Likelihood Method. Acta Crystallographica D, 53, 240-255). In all the structure there is a cis-Pro at 110.

In the C249S-citrulline and C249S-ADMA structures, all the residues are within allowed regions of the Ramachandran plot except E171, which precedes the phi-positive H172. These residues are in a loop region, but the electron density is unambiguous, although no functional reason has yet been discerned.

Results

The DDAH fold

The crystal structure of DDAH from Pseudomonas aeruginosa residues 0 to 254 as the selenomethioinine derivative was solved by MAD methods at 2.4 Å resolution. Amino-terminal residues that had been cloned to the N terminus are not seen in the electron density map. The phases from the Se-substructure were used to solve the native enzyme at 2.4 Å resolution. Coordinates from the native structure were used as the model to solve the structures of a C249S mutant co-crystallized with ADMA at 2.0 Å resolution and with citrulline at 1.8 Å resolution by molecular replacement. The structure coordinates for the native PaDDAH are set out in Table II and the structure coordinates of C249S co-crystallized with citrulline are set out in Table III.

The overall fold of Pseudomonas aeruginosa DDAH (PADDAH) shown comprises a type of barrel formed from 5 modules of a weakly conserved ββαβ structural motif which enclose the active site. The N— and C-termini protrude from one (“bottom”) end of the barrel, and at the other (“top”) end loop regions surround the active site entrance. Within each β₁β₂αβ₃ module the three strands are approximately parallel to the barrel axis and arranged as a sheet with β₁ (inside the barrel) antiparallel to β₂, which in turn is parallel to β₃ (on the outside of the barrel); the helix lies on a face of the sheet. There are more or less elaborate loops between these secondary structural elements.

Module 1 comprises residues 0-67, and the C-terminal strand 249-254, acting as β₁; module2, residues 68-117; module3, residues 118-166; module 4, residues 167-205; and module 5(residues 206-248).

As predicted by both inspection of alignment from PSIBLAST (Altschul et al., 1997, Gapped BLAST and PSI-BLAST: a new generation of protein database search programs. Nucleic Acids Res. 25, 3389-3402.) and the 3D-PSSM fold prediction server (Kelley et al., 2000, Enhanced genome annotation using structural profiles in the program 3D-PSSM. J. Mol. Biol. 299, 501-522), the DDAH fold is related to that of human L-argine:glycine amindinotransferase (AT, DPB code 1jdw), although PaDDAH is much smaller (254 as opposed to 360 amino acid residues) and the sequence identity between human DDAH1 and AT is 18%. A superposition of the Cα traces of PaDDAH and AT is shown in; the rms based on 217 equivalent Cα atoms is 1.8 Å. Module 4 is particularly sketchy, with the alpha helix represented by four residues in a 3₁₀ helical conformation. However there is a clear correspondence between the catalytic residues of AT (C407, H303, D254) and C249, H162 and E114 of DDAH. Interestingly, these residues and the mechanistically important D66 are all found to lie in roughly the same position with respect to the module-structure, on a kinked region immediately preceding strand β₁ at the same level inside the barrel.

Dimer Formation

Both AT and DDAH are dimeric in the crystal, and DDAH has been shown to be dimeric in solution by analytical gel-filtration and dynamic light scattering (see Experimental section). In each of the crystal forms in the present study the dimer is formed in the same manner, so that it is less likely to be a crystallisation artefact. At the heart of each dimer is the interaction of a strand on the exterior of the barrel with its equivalent around a two-fold axis. In DDAH this is strand β₃ of module 1 (residues 48 to 53), whereas in AT it is strand β₃ of module 2 (residues 209-212), leading to different spatial arrangements of the protomers in their respective dimers. Calculation of accessible surface areas for DDAH monomers and dimers in GRASP give a mean buried area per protomer at the interface of 1154 Å², which is within the limits for a protein of this molecular weight (Jones and Thornton, 1995, Protein-Protein Interactions: A Review of Protein Dimer Structures In Progress in Biophysics and Molecular Biology, 63, 31-165.). The AT interface is more elaborate than that of DDAH, and involves additional residues from modules 1 and 3 (Humm et al., 1997, Crystal structure and mechanism of human L-arginine:glycine amidinotransferase: a mitochondrial enzyme involved in creatine biosynthesis. EMBO J. 16, 3373-85.); the surface area/protomer buried on dimer formation is 1773 Å² in AT.

The angle between the approximate 5-fold axis in the barrel and the dimer axis is different in AT and DDAH. In AT these-axes are almost parallel, and it was suggested that this enables the N-terminal residues to protrude on the same side of the dimer and to act as a membrane anchor. In DDAH the two-fold axis is more tilted with respect to the barrel axis, so that the chain termini are more widely separated, which is consistent with its predominantly cyctosolic location (Birdsey et al., 2000, Intracellular localization of dimethylarginine dimethylaminohydrolase overexpressed in an endothelial cell line. Acta Physiol. Scand. 168, 73-79.).

The angle between the two interface strands is likewise significantly different in AT and DDAH. In DDAH there are two pairs of hydrogen bonds across the two-fold axis, so that a 6-membered beta sheet is formed form the two adjacent 3-stranded β₁β₂β₃ sheets of the protomers. In AT the angle between the two strands is too large for continuous beta sheet formation, and the central strands interact at a single residue, with most of the interface contacts contributed by residues from parts of the structure not present in DDAH.

A similar variation in homodimers of very similar enzyres is reported in a structure of a prokaryotic superoxide dismutase (SOD) compared with the eukaryotic SODs (Bourne et al., 1996, Novel dimeric interface and electrostatic recognition in bacterial Cu, Zn superoxide dismutase. Prog. Natl. Acad. Sci. USA 93, 12774-12779.). Both enzymes have the same fold and function but different strands from the Greek key β-barrel are used in the two interfaces, and different electrostatic substrate guidance is postulated. DDAH and AT are much more distantly related than the two form of SOD, but there is the intriguing question of whether they diverged from a common ancestor by the formation of two different dimers in response to different cellular localisation as well as different substrate specificity.

The DDAH Active Site

The active site of the SeMet derivative shows no density unaccounted for by protein atoms. The native form has density that may be β-mercaptoethanol, analogous to that found in one of the preparations of AT (PDB code 1jdw). The citrulline co-crystallisation experiment contains a very clear citrulline molecule in difference density where it had not been included in the model. The ADMA co-crystallisation experiment gave crystals with four molecules in the asymmetric unit.

Much of the structure could be built into an averaged map, and the averaged difference map for the ligand shows the methyl groups of ADMA to be present. However, on refinement they have somewhat higher temperature factors than the rest of the ADMA molecule, which may be the result either of movement, or of a slow turnover of the ADMA resulting in the active sites being occupied partly by ADMA and partly by the product, citruline. As might be expected there is no evidence of dimethylamine, the other product of the reaction.

Ligand Binding in DDAH

Identification of the active site residues permitted the production of the mutants C249S, H162F and E114Q. All three mutants were shown to be inactive by in vitro assays.

Co-crystallisation of the active site mutant C249S PaDDAH with ADMA (substrate) and citrulline (product/inhibitor) has provided evidence for the binding modes at the extreme of the reaction. Comparison of the binding site with that of arginine in AT shows that the ligand has changed orientation with respect to the active site cavity by about 80°, preserving the orientation of the scissile bond. For this reason we propose a similar mechanism, involving nucleophilic attack by the S atom of C249 on the carbon atom of the guanidino group of dimethylarginine. The slight deviation from planarity of the substituted guanidino group may be induced by the binding of the substrate in the active site, and would increase the susceptibility of this bond to cleavage.

The DDAH site lies in a negatively charged cleft at the centre of the barrel. In the apo-form of the enzyme this site is open but In the presence of ligand, either substrate (ADMA) and product/inhibitor (citrulline), the active site is occluded by the folding down of a loop between residues 14 and 27 whose extremity is H22. This flap is very mobile/disordered in the apo-form of the protein, but becomes more ordered, with a concomitant increase in resolution of the diffraction, on binding ADMA or citrulline. There is a single direct contact between the ligand (ADMA or citrulline) and the flap: the carbonyl O of Leu 18 binds the peptide N of ADMA. The sidechain of D60 also bridges between N of ADMA, and N of L18 (does in ADMA-check CIT. The flap region is quite variable amongst DDAHs from different species, and there is a one-residue insertion here in mammalian DDAH compared with PADDAH, so this contact is presumably a late event in ligand binding, rather than a driving force. This flap movement is in an opposite sense from that in AT, where the binding of L-arginine induces an open conformation.

The catalytic residues are supplied by three different modules of the sequence: E114 by module 3, H162 by module 4 and C249 by module 5. They all lie in the linker regions between strand β₃ of one their motif and strand β₁ of the next. E114 and H162 immediately precede small 3₁₀ helices, and C249 is also in a helical conformation.

Within the active site, a network of hydrogen bonds holds the ligand in place, with the C249 of the catalytic triad on one side, and H162, E114 on the other. The peptide end of the molecule form is hydrogen bonds to the O atoms of L18 and I243, and to the sidechains of R132 and D60. The hydrophobic part of the DDAH extended backbone is protected from the charged residue around the cleft by the sidechains of F63 and L161. The guanidinium end of the ADMA is hydrogen bonded to D66, which bridges Ne and the non-methylated N^{G}′, and the H-bonding is similar in the citrulline complex.

There is an extensive network of hydrogen bonds surrounding the active site cleft, which stabilises the relationships between the secondary structure elements, the ligand and the catalytic residues.

Comparison of the DDAH Active Site with that of Amidinotransferase

Having the scissile bond in the same orientation in the DDAH and AT complexes means that the backbones of the two ligands are differently disposed with respect to the active site residues, with an angle between them of about 80 deg. This also has the result that some residues, while apparently conserved between the two enzymes, actually have slightly different roles.

D66(PaDDAH) and its sequential equivalent D170(AT) both make contact with their ligand, but D66(PADDAH) makes two hydrogen bonds rather than one, effectively locking the guanidino group in place. The D at this position is conserved in all DDAH sequences except M tuberculosis, and in the AT family. D66 is held in place by R85 (R189) and E88 (E192). R85 and E88 are conserved in all know DDAH and AT sequences.

The equivalent dual hydrogen-bonding function to D66 is carried out by D305 in AT, which is conserved in known AT sequences. The sequentially equivalent residue to AT's D305 in PADDAH is R164, which is K or R in known DDAH sequences (except M tuberculosis), and whose side-chain does not make contact with the ligand. The sidechain of E65 forms a hydrogen bond to NG′ of ADMA, and is mainly conserved in the DDAH sequences; this aligns with the conserved R169 in AT, whose sidechain is directed awat from the arginine ligand; the equivalent bond is formed by D170 of AT, which is conserved in that family.

Residues involved in hydrogen bonding to the peptide end of the ligand are quite different in DDAH and AT. Interactions with the mainchain of L18 are mentioned above, another example is D60, whose sidechain in hydrogen-bonded to the N of ADMA, but the equivalent Y164 in AT is not in contact with the R ligand.

Although the ligand in each complex makes contacts with two hydrophobic sidechains, the residues involved are different: L161 and F63 in DDAH; M302 and L358 in AT.

Proposed Mechanism

Ogawa et al. (1989, Purification and properties of a new enzyme, N^{G},N^{G}-dimethylarginine dimethylaminohydrolase, from rat kidney. J. Biol. Chem., 264, 10205-9) showed that DDAH from rat kidney irreversibly converted one molecule of ADMA to one molecule each of L-citrulline and dimethylamine, requiring no co-factors and giving no other product. In the presence of H₂¹⁸O, ¹⁸O was incorporated onto the ureido group of the citrulline, showing that a hydrolytic step is involved. They showed that somebut not all, SH-blocking agents acted as inhibitors, as did certain divalent metal ions. The structural data suggested that in PaDDAH the catalytic residues are C249, H162 and E114, and this was confirmed by mutations at these three positions which abolished activity. In most of the DDAH sequences the equivalent of E114 is D, but comparison with the At structure, where D is also used, suggests that only minor adjustments are required to allow for the differing sidechain lengths. In the discussion which follows it can be assumed that D could replace E114 without major changes to the mechanism.

The ADMA (substrate) and citrulline (product/inhibitor) are both fixed firmly into the active site by hydrogen bonds. If we denote the methyl-bearing guanidino-nitrogen atom as N^{G}, and the hydrogen bearing nitrogen as N^{G}′, the sidechain of D66 hydrogen bonds to both the NE and N^{G′} in both complexes. N^{G′} is also hydrogen-bonded to the sidechain of E65 and both E65 and D66 are held in place by further H-bonds. It is suggested that these bonds serve as a vice to hold the substrate in position during the reaction. The OG of S249 (and therefore the SG of native enzyme), the CZ of the substrate and the ND of H162 are in a line perpendicular to the plane of the guanidino group.

We hypothesise that the active site Cys and His form a thiolate-imidazoliuniurn ion pair analogous to that of the cysteine proteases (Storer and Menard, 1994, Catalytic Mechanism in papain family of cysteine peptidases. Methods Enzymol., 244, 486-500).

The proposed mechanism then involves nucleophilic attack by SG of C249 on the CZ of the substrate with formation of tetrahedral centres at both CZ and N^{G}, which carries. The electron density for the ADMA show some degree of distortion from planarity here, which would imply a loss of conjugation of the bonds of the guanidino group. Although it is difficult to apportion between twisting and non-planarity at the CZ, N^{G} atoms, it is suggested that the effect would be to increase the reactivity of this bond.

Dimethylamine is released, and diffuses out of the active site, leaving the thiourea oxyanion derivative. In the second part of the reaction, hydrolysis of the intermediate, possibly by a water molecule activated by interaction with H162 (there is no obvious general base in the vicinity). Finally collapse of the tetrahedral centre and release of a proton leaves the citrulline bound to active site; its release presumably require a conformational change back to the open form of the enzyme. There is as yet, no definite proof for the formation of an intermediate, and a one-step reaction could be envisaged, but this distinction will require direct observation of an intermediate.

DDAH Specificity

Both ADMA and LMMA will fit into the binding site in DDAH, and both will susceptible to the steric distortion that is proposed to assist in activating the scissile bond. Introduction of SDMA, however, would be hindered both sterically and by the negatively charged environment occupied by the non-methylated N^{G} of ADMA.

PADDAH has been shown to have weak deiminase activity (Santa Maria et al, 1999, Identification of microbial dimethylarginine dimethylarninohydrolase enzymes. Mol. Microbiol., 33, 1278-1279). It is quite possible to fit arginine in place of ADMA in the active site of DDAH, and it might be expected to bind in the same way. However, the active site of arginine deiminase (see below) is likely to be considerably different from that of DDAH.

While non-methylated arginine would fit into the bonding pocket, there would be space for one or more water molecules, as found in the citrulline structure, and also there would be less tendency for the guanidinium group to twist to fit in the active site. A closer fit of local residues and the assumption is that a chemical requirement for one of the arginine N^{G} atoms to become the leaving group is not optimised in DDAH.

Relationship of the PaDDAH Structure to other Family Members

Two questions that arise in considering the relationship of the structure of the active site of PADDAH to other structures in the family, and its implications for other sequences in the superfamily are (i)Is PaDAAH a good model for human/mammalian DDAH? and (ii) What might the arginine deiminase active site look like? Some of the key residues around the PADDAH active site, together with their equivalents in arginine:glycine amidinotransferase, arginine:inosamine phosphate arnidinotransferase, the mammalian DDAH isoforms I and II and arginine deiminase are listed in Table IV.

(i)Is PaDAAH a good model for human/mammalian DDAH?

In a sequence-based alignment, while the catalytic His and D/E are in register, the catalytic Cys of DDAHII is two residues later than that of the bacterial and DDAHI, so that an adjustment to the preceding loop will be required. This is in contact with the flap region, and may somewhat change its position. The flap region sequence is not conserved in the DDAH sequences, but since the only requirement is for contact of the ligand with a mainchain atom, this is not unexpected. There are, however, quite major large insertions and deletions, particularly in modules 4 and 5, which are likely lead to local structural differences between PaDDAH and mammalian DDAH, while maintaining the overall architecture.

Around the active site, most of the PaDDAH residues are conserved in DDAHI and bacterial DDAH. There are one or two surprising positions amongst these residues at which there is no conservation between PaDDAH and the DDAHI isoforms. Two obvious examples are-E65, which is G in DDAHII and D60, which is L in DDAHII. R132 in DDAHI and bacterial DDAH is W in DDAII so clearly this is not an essential residue. Futhermore, 132 is spatially close to another mutated residue between DDAHI and 2, G81W, and some degree of repacking will be needed to accommodate these two sidechains in DDAH2.

Another feature reported in mammalian DDAH, but not PaDDAH is the presence of a zinc ion, whose function is was speculated to be structural; Zn²⁺ also inhibits the enzymatic activity (Bogumil et al., 1998, Characterization of dimethylargininase from bovine brain: evidence for a zinc binding site. Biochemistry 37, 4791-4798.). No evidence of a Zn²⁺ ion was found in any of the present-structures. The Zn²⁺ is thought to bind to cysteine and histidine sidechains, and there are several places where a residue is conserved as one of these in mammalian DDAH but not in the bacterial sequences, including 35, 168, 190, 205 and 214 (PADDAH numbering). Apart from 35, all of these residues lie in parts of the structure where there are insertions/deletions, and thus expected structural variations, between the PaDDAH and mammalian sequences. So far inspection of the sequences and structure has failed to reveal a possible location for this binding site, although in view of its inhibiting effect it is possible binding involves at least one residue from the active site.

(ii) What M the Arginine Deimnase Active Site look like?

Arginine deiminase is a bacterial enzyme that catalyses the first step in the arginine dihydrolase pathway, which is an important source of energy in microbes. It catalyses the hydrolysis of arginine to citrulline and ammonia, which involves breaking the equivalent bond to that broken by DDAH. The substrates are not interchangeable, however; rat DDAH was reported to have no arginine deiminase activity (Ogawa et al., 1989, supra) and PADDAH to have only a very low level (Santa Maria et al., 1999, supra).

Inspection of the aligned deiminase sequences (Knodler et al., 1998, Clonging and expression fo a prokaryotic enzyme, Arginine deiminase, from a primitive eukaryote Giardia intestinalis. J. Biol. Chem. 273 4470-4477) shows conserved Cys, His and Glu residues in the same order as in DDAH so that it is reasonable to assume that deiminase has a similar catalytic triad and mechanism to DDAH. Homology between DDAH and arginine deiminase has been noted (Leiper et al., 1999, supra), although it is largely confined to the residues between the catalytic Glu and His residues and those immediately adjacent to the catalytic Cys. The sequence length in module 2 is approximately the same as that in PADDAH, but there is a large insertion in module 4.

Table IV shows that the arginine deiminase active site has some of the characteristics of both the DDAH and AT site.

A preliminary test for modelling the deiminase active site was made by taking the structure of PaDDAH and making local mutations (residues in brackets are conserved from PaDDAH to PaDEIM); D60N (63F) E65R (66D) (85R) (88E) 117(D) (132R) L161M K164D. The pair of mutations E65R and K164D where the R and D are conserved in the deiminases, are characteristic of the AT rather than the DDAH active site is particularly notable. These sidechains occupy rather different spatial positions in DDAH and AT, so that a rearrangement more complicated than reversal of hydrogen-bonded pair is possible for deiminase. R132 is conserved, and might be thought to have the same function as in the PaDDAH active site; however, the equivalent residue to R132 in human and mouse DDAHII is W, so this position may not be critical.

Other mutations which on inspection of the substitutions above seem to be affected by the changes around active site were Y38L, A82W, N204, 1206Q, V247G, S248H, S251T, L252Cand R253P but of these only V247G represents a change to a residue conserved in the deiminase family. From graphical inspection of the mutations above, and bearing in mind the differing length of putative loop regions of the molecules, it is quite possible that arginine deiminase has its own unique variant of the active site, possibly with a different flap region and a distinctive orientation of the substrate.

How Does This Structure Compare With Others With a Cys-His-Asp Triad?

The catalytic triad of DDAH is reminiscent of that of the cysteine proteases; however, the secondary structure elements in which the are found do not resemble the cysteine proteases. It is therefore somewhat surprising that manual overlay (using O) of the sidechains of the catalytic triad of DDAH with that of arylamine N-acetyl transferase—a member of the cysteine protease superfamily (Sinclair et al., 2000, Structure of arylamin N-acetyltransferase reveals a catalytic triad. Nature Struct. Biol. 7, 560-564) -shows that distances between the three equivalent pairs of Cα atoms of 2.9, 2.8 and 1.8 A. However there is no general alignment of secondary structural elements, and the sequence order of the catalytic residues is reversed, so it not likely that they share a common ancestry. This seems to be an example of convergent evolution of a catalytic triad in the cysteine protease superfamily, analogous to the relationship between the serine proteases and other enzymes that have independently evolved a Ser-His-Asp catalytic triad (Dodson, G. and Wlodawer, A, 1998, Catalytic triads and their relatives. TIBS 23, 347-352).

How Does it Compare With Other Enzymes that Process Arginine and Related Molecules?

Of the many enzymes involved in arginine synthesis and metabolism (Wu and Morris, 1998, Arginine Metabolism: nitric oxide and beyond. Biochem. J. 336, 1-17) structures are known for NOS (Fischmann et al., 1999, Structural characterization of nitric oxide synthase isoforms reveals striking active-site conservation, Nat Struct Biol. 6, 233-42), arginase (Cox et al., 1999, Arginase-boronic acid complex highlights a physiological role in erectile function, Nat Struct Biol. 6, 1043-1047), PRMT3 (Zhang et al., 2000, Crystal structure of the conserved core of protein arginine methyltransferase PRMT3, EMBO J 19, 3509-3519), argininosuccinate lyase (Turner et al., 1997, Human argininosuccinate lyase: a structural basis for intragenic complementation, Proc Natl Acad Sci USA 94,9063-8) and AT but apart from AT none bears any resemblance to that of DDAH. There are two enzymes which deiminate arginine, arginine deiminase (EC 3.5.3.6) which acts on free substrate, and which is so far only known so far in bacteria, and protein arginine deiminase (PAD, EC 3.5.3.15), found mainly in vertebrates, which converts arginine residues in proteins to citrulline in the presence of calcium ion. While DDAH processes free methylarginines, there is so far no equivalent enzyme known which processes methylarginine residues in a protein context.

There appears to be little similarly between DDAH and protein arginine deiminase, which is not surprising since DDAH buries the substrate, and in a protein context this would not be possible. It has been suggested the PAD has a catalytic Cys residue (McGraw et al., 1999, Purification,characterization, and sequence analysis of a potential virulence factor from Porphyromonas gingivalis, peptidylarginine deiminase. Infection and Immunity 67, 3248-3256), and the presence of a similar catalytic triad seems quite plausible.

TABLE I
X-ray crystallographic data collection
Each data set collected on a single cryo-cooled crystal at flash
cooled to 100 K in a gaseous nitrogen stream: the cryoprotetant was
glycerol for the native dataset and N-paratone for all the others.

| Compound | SeMet | Native | C249S + citrulline | C249S + ADMA |
|---|---|---|---|---|
| Beamline | Grenoble BM30 | Daresbury 9.5 | Daresbury 14.1 | Daresbury 14.1 |
| Detector | Mar345 | Mar345 | ADSC | ADSC |
| Wavelength(Å) | λ1 0.98001 | 1.2 | 1.244 | 1.244 |
| | λ2 0.98048 | | | |
| | λ3 0.96686 | | | |
| Spacegroup | P21212 | P21212 | C2 | P21 |
| Cell dimensions | | | | |
| a (Å) | 86.367 | 86.030 | 69.814 | 85.020 |
| b (Å) | 127.872 | 127.720 | 61.798 | 123.10 |
| c (Å) | 47.723 | 47.340 | 54.618 | 45.804 |
| β (deg) | | | 95.146 | 95.742 |
| Z | 2 | 2 | 1 | 4 |
| Resolution (Å) | 2.52 | 2.37 | 1.8 | 2.0 |
| Unique refls. | λ1 15665 | 18992 | 20847 | 62421 |
| | λ2 15417 | | | |
| | λ3 15887 | | | |
| Av.redundancy | λ1 3.5 (2.3) | 3.0 (2.2) | 3.5 (3.5) | 7.4 (7.2) |
| | λ2 3.3 (2.2) | | | |
| | λ3 | | | |
| Completeness % | λ1 97.4 (85.6) | 97.5 (87.2) | 99.3 (99.8) | 98.9 (98.3) |
| | λ2 97.0 (84.9) | | | |
| | λ3 97.7 (90.8) | | | |
| R_{merge} | λ1 0.026 (0.062) | 0.054 (0.243) | 0.045 (0.082) | 0.056 (0.114) |
| | λ2 0.025 (0.025) | | | |
| | λ3 0.028 (0.060) | | | |
| Ranom | λ1 0.042 (0.070) | | | |
| | λ2 0.025 (0.055) | | | |
| | λ3 0.036 (0.059) | | | |
| I/σ(I) | λ1 16.3 (8.8) | 14.3 (4.9) | 10.0 (8.0) | 9.3 (5.9) |
| | λ2 13.4 (4.2) | | | |
| | λ3 14.9 (4.9) | | | |

TABLE II
Structural Coordinates for the native PaDDAH

CRYST1 86.030 127.720 47.340 90.00 90.00 90.00

| | | | | |
|---|---|---|---|---|
| SCALE1 | 0.011624 | 0.000000 | 0.000000 | 0.00000 |
| SCALE2 | 0.000000 | 0.007830 | 0.000000 | 0.00000 |
| SCALE3 | 0.000000 | 0.000000 | 0.021124 | 0.00000 |

| | | | | | | | | | | | | |
|---|---|---|---|---|---|---|---|---|---|---|---|---|
| ATOM | 1 | N | PHE | A | 0 | 37.110 | 33.687 | 30.450 | 1.00 | 62.68 | 7 | N |
| ATOM | 2 | CA | PHE | A | 0 | 36.149 | 32.847 | 29.678 | 1.00 | 62.98 | 6 | C |
| ATOM | 3 | C | PHE | A | 0 | 36.358 | 32.903 | 28.163 | 1.00 | 61.43 | 6 | C |
| ATOM | 4 | O | PHE | A | 0 | 37.132 | 33.660 | 27.605 | 1.00 | 62.94 | 8 | O |
| ATOM | 5 | CB | PHE | A | 0 | 34.703 | 33.264 | 29.966 | 1.00 | 63.80 | 6 | C |
| ATOM | 6 | CG | PHE | A | 0 | 34.173 | 34.358 | 29.076 | 1.00 | 63.61 | 6 | C |
| ATOM | 7 | CD1 | PHE | A | 0 | 33.061 | 34.131 | 28.298 | 1.00 | 63.56 | 6 | C |
| ATOM | 8 | CD2 | PHE | A | 0 | 34.764 | 35.610 | 29.062 | 1.00 | 63.57 | 6 | C |
| ATOM | 9 | CE1 | PHE | A | 0 | 32.527 | 35.152 | 27.535 | 1.00 | 62.87 | 6 | C |
| ATOM | 10 | CE2 | PHE | A | 0 | 34.202 | 36.637 | 28.329 | 1.00 | 64.41 | 6 | C |
| ATOM | 11 | CZ | PHE | A | 0 | 33.068 | 36.408 | 27.574 | 1.00 | 64.35 | 6 | C |
| ATOM | 12 | N | MET | A | 1 | 35.603 | 32.062 | 27.439 | 1.00 | 58.44 | 7 | N |
| ATOM | 13 | CA | MET | A | 1 | 35.783 | 32.081 | 25.998 | 1.00 | 53.42 | 6 | C |
| ATOM | 14 | C | MET | A | 1 | 34.449 | 32.047 | 25.292 | 1.00 | 48.80 | 6 | C |
| ATOM | 15 | O | MET | A | 1 | 33.389 | 31.853 | 25.845 | 1.00 | 50.03 | 8 | O |
| ATOM | 16 | CB | MET | A | 1 | 36.454 | 30.804 | 25.498 | 1.00 | 57.14 | 6 | C |
| ATOM | 17 | CG | MET | A | 1 | 37.951 | 30.805 | 25.702 | 1.00 | 61.29 | 6 | C |
| ATOM | 18 | SD | MET | A | 1 | 38.708 | 29.417 | 24.809 | 1.00 | 66.99 | 16 | S |
| ATOM | 19 | CE | MET | A | 1 | 39.150 | 30.325 | 23.302 | 1.00 | 65.96 | 6 | C |
| ATOM | 20 | N | PHE | A | 2 | 34.574 | 32.175 | 23.991 | 1.00 | 43.54 | 7 | N |
| ATOM | 21 | CA | PHE | A | 2 | 33.416 | 32.043 | 23.105 | 1.00 | 37.19 | 6 | C |
| ATOM | 22 | C | PHE | A | 2 | 33.709 | 30.752 | 22.331 | 1.00 | 35.23 | 6 | C |
| ATOM | 23 | O | PHE | A | 2 | 34.874 | 30.579 | 21.886 | 1.00 | 32.49 | 8 | O |
| ATOM | 24 | CB | PHE | A | 2 | 33.335 | 33.214 | 22.123 | 1.00 | 37.42 | 6 | C |
| ATOM | 25 | CG | PHE | A | 2 | 32.819 | 34.501 | 22.704 | 1.00 | 33.48 | 6 | C |
| ATOM | 26 | CD1 | PHE | A | 2 | 33.458 | 35.678 | 22.366 | 1.00 | 29.44 | 6 | C |
| ATOM | 27 | CD2 | PHE | A | 2 | 31.711 | 34.474 | 23.575 | 1.00 | 33.10 | 6 | C |
| ATOM | 28 | CE1 | PHE | A | 2 | 33.020 | 36.893 | 22.877 | 1.00 | 27.39 | 6 | C |
| ATOM | 29 | CE2 | PHE | A | 2 | 31.274 | 35.726 | 24.095 | 1.00 | 34.33 | 6 | C |
| ATOM | 30 | CZ | PHE | A | 2 | 31.934 | 36.904 | 23.735 | 1.00 | 30.88 | 6 | C |
| ATOM | 31 | N | LYS | A | 3 | 32.668 | 29.943 | 22.181 | 1.00 | 32.94 | 7 | N |
| ATOM | 32 | CA | LYS | A | 3 | 32.808 | 28.711 | 21.400 | 1.00 | 32.59 | 6 | C |
| ATOM | 33 | C | LYS | A | 3 | 32.065 | 28.749 | 20.052 | 1.00 | 35.08 | 6 | C |
| ATOM | 34 | O | LYS | A | 3 | 32.259 | 28.005 | 19.052 | 1.00 | 31.22 | 8 | O |
| ATOM | 35 | CB | LYS | A | 3 | 32.431 | 27.447 | 22.171 | 1.00 | 30.87 | 6 | C |
| ATOM | 36 | CG | LYS | A | 3 | 33.123 | 27.110 | 23.406 | 0.00 | 25.00 | 6 | C |
| ATOM | 37 | CD | LYS | A | 3 | 32.330 | 26.122 | 24.242 | 0.00 | 25.00 | 6 | C |
| ATOM | 38 | CE | LYS | A | 3 | 33.209 | 24.995 | 24.748 | 0.00 | 25.00 | 6 | C |
| ATOM | 39 | NZ | LYS | A | 3 | 32.549 | 24.234 | 25.844 | 0.00 | 25.00 | 7 | N |
| ATOM | 40 | N | HIS | A | 4 | 31.068 | 29.690 | 19.935 | 1.00 | 35.39 | 7 | N |
| ATOM | 41 | CA | HIS | A | 4 | 30.260 | 29.860 | 18.772 | 1.00 | 30.13 | 6 | C |
| ATOM | 42 | C | HIS | A | 4 | 29.958 | 31.280 | 18.304 | 1.00 | 30.91 | 6 | C |
| ATOM | 43 | O | HIS | A | 4 | 29.732 | 32.268 | 18.938 | 1.00 | 28.72 | 8 | O |
| ATOM | 44 | CB | HIS | A | 4 | 28.797 | 29.462 | 19.108 | 1.00 | 34.36 | 6 | C |
| ATOM | 45 | CG | HIS | A | 4 | 28.839 | 28.018 | 19.476 | 1.00 | 39.64 | 6 | C |
| ATOM | 46 | ND1 | HIS | A | 4 | 29.336 | 27.101 | 18.573 | 1.00 | 38.50 | 7 | N |
| ATOM | 47 | CD2 | HIS | A | 4 | 28.466 | 27.371 | 20.612 | 1.00 | 39.75 | 6 | C |
| ATOM | 48 | CE1 | HIS | A | 4 | 29.267 | 25.923 | 19.138 | 1.00 | 38.43 | 6 | C |
| ATOM | 49 | NE2 | HIS | A | 4 | 28.752 | 26.067 | 20.355 | 1.00 | 40.69 | 7 | N |
| ATOM | 50 | N | ILE | A | 5 | 29.800 | 31.417 | 17.005 | 1.00 | 31.23 | 7 | N |
| ATOM | 51 | CA | ILE | A | 5 | 29.389 | 32.546 | 16.247 | 1.00 | 31.51 | 6 | C |
| ATOM | 52 | C | ILE | A | 5 | 28.206 | 32.243 | 15.338 | 1.00 | 30.53 | 6 | C |
| ATOM | 53 | O | ILE | A | 5 | 28.084 | 31.249 | 14.667 | 1.00 | 30.67 | 8 | O |
| ATOM | 54 | CB | ILE | A | 5 | 30.530 | 33.085 | 15.378 | 1.00 | 34.30 | 6 | C |
| ATOM | 55 | CG1 | ILE | A | 5 | 31.276 | 34.062 | 16.341 | 1.00 | 34.58 | 6 | C |
| ATOM | 56 | CG2 | ILE | A | 5 | 30.004 | 33.777 | 14.108 | 1.00 | 33.67 | 6 | C |
| ATOM | 57 | CD1 | ILE | A | 5 | 32.606 | 34.306 | 15.607 | 1.00 | 39.67 | 6 | C |
| ATOM | 58 | N | ILE | A | 6 | 27.264 | 33.192 | 15.221 | 1.00 | 28.58 | 7 | N |
| ATOM | 59 | CA | ILE | A | 6 | 26.134 | 33.025 | 14.300 | 1.00 | 23.15 | 6 | C |
| ATOM | 60 | C | ILE | A | 6 | 26.183 | 34.313 | 13.472 | 1.00 | 22.48 | 6 | C |
| ATOM | 61 | O | ILE | A | 6 | 26.466 | 35.446 | 13.947 | 1.00 | 23.13 | 8 | O |
| ATOM | 62 | CB | ILE | A | 6 | 24.759 | 32.927 | 14.927 | 1.00 | 25.16 | 6 | C |
| ATOM | 63 | CG1 | ILE | A | 6 | 24.637 | 31.732 | 15.868 | 1.00 | 21.17 | 6 | C |
| ATOM | 64 | CG2 | ILE | A | 6 | 23.681 | 33.013 | 13.829 | 1.00 | 21.42 | 6 | C |
| ATOM | 65 | CD1 | ILE | A | 6 | 23.301 | 31.698 | 16.628 | 1.00 | 24.74 | 6 | C |
| ATOM | 66 | N | ALA | A | 7 | 26.027 | 34.143 | 12.153 | 1.00 | 19.06 | 7 | N |
| ATOM | 67 | CA | ALA | A | 7 | 26.108 | 35.297 | 11.282 | 1.00 | 15.97 | 6 | C |
| ATOM | 68 | C | ALA | A | 7 | 25.239 | 35.074 | 10.083 | 1.00 | 17.69 | 6 | C |
| ATOM | 69 | O | ALA | A | 7 | 24.846 | 33.925 | 9.978 | 1.00 | 16.09 | 8 | O |
| ATOM | 70 | CB | ALA | A | 7 | 27.524 | 35.450 | 10.763 | 1.00 | 16.42 | 6 | C |
| ATOM | 71 | N | ARG | A | 8 | 24.985 | 36.082 | 9.225 | 1.00 | 20.62 | 7 | N |
| ATOM | 72 | CA | ARG | A | 8 | 24.109 | 35.758 | 8.053 | 1.00 | 21.09 | 6 | C |
| ATOM | 73 | C | ARG | A | 8 | 24.553 | 36.478 | 6.782 | 1.00 | 20.84 | 6 | C |
| ATOM | 74 | O | ARG | A | 8 | 24.839 | 37.702 | 6.814 | 1.00 | 17.32 | 8 | O |
| ATOM | 75 | CB | ARG | A | 8 | 22.666 | 36.241 | 8.279 | 1.00 | 23.39 | 6 | C |
| ATOM | 76 | CG | ARG | A | 8 | 21.636 | 35.945 | 7.159 | 1.00 | 23.03 | 6 | C |
| ATOM | 77 | CD | ARG | A | 8 | 20.301 | 36.641 | 7.518 | 1.00 | 24.04 | 6 | C |
| ATOM | 78 | NE | ARG | A | 8 | 19.582 | 36.087 | 8.651 | 1.00 | 25.68 | 7 | N |
| ATOM | 79 | CZ | ARG | A | 8 | 18.971 | 34.885 | 8.561 | 1.00 | 24.76 | 6 | C |
| ATOM | 80 | NH1 | ARG | A | 8 | 19.088 | 34.267 | 7.381 | 1.00 | 27.28 | 7 | N |
| ATOM | 81 | NH2 | ARG | A | 8 | 18.281 | 34.175 | 9.395 | 1.00 | 21.38 | 7 | N |
| ATOM | 82 | N | THR | A | 9 | 24.625 | 35.788 | 5.650 | 1.00 | 19.75 | 7 | N |
| ATOM | 83 | CA | THR | A | 9 | 25.056 | 36.555 | 4.443 | 1.00 | 23.25 | 6 | C |
| ATOM | 84 | C | THR | A | 9 | 24.210 | 37.745 | 4.160 | 1.00 | 22.64 | 6 | C |
| ATOM | 85 | O | THR | A | 9 | 23.013 | 37.687 | 4.351 | 1.00 | 23.85 | 8 | O |
| ATOM | 86 | CB | THR | A | 9 | 24.870 | 35.591 | 3.226 | 1.00 | 23.90 | 6 | C |
| ATOM | 87 | OG1 | THR | A | 9 | 25.514 | 34.403 | 3.785 | 1.00 | 27.68 | 8 | O |
| ATOM | 88 | CG2 | THR | A | 9 | 25.578 | 35.942 | 1.975 | 1.00 | 21.05 | 6 | C |
| ATOM | 89 | N | PRO | A | 10 | 24.703 | 38.834 | 3.703 | 1.00 | 24.41 | 7 | N |
| ATOM | 90 | CA | PRO | A | 10 | 23.827 | 39.962 | 3.389 | 1.00 | 23.79 | 6 | C |
| ATOM | 91 | C | PRO | A | 10 | 23.042 | 39.664 | 2.134 | 1.00 | 28.09 | 6 | C |
| ATOM | 92 | O | PRO | A | 10 | 23.514 | 39.037 | 1.162 | 1.00 | 32.99 | 8 | O |
| ATOM | 93 | CB | PRO | A | 10 | 24.760 | 41.053 | 3.035 | 1.00 | 19.47 | 6 | C |
| ATOM | 94 | CG | PRO | A | 10 | 26.019 | 40.404 | 2.602 | 1.00 | 20.50 | 6 | C |
| ATOM | 95 | CD | PRO | A | 10 | 26.148 | 39.109 | 3.434 | 1.00 | 24.16 | 6 | C |
| ATOM | 96 | N | ALA | A | 11 | 21.828 | 40.167 | 2.084 | 1.00 | 31.10 | 7 | N |
| ATOM | 97 | CA | ALA | A | 11 | 20.890 | 40.076 | 0.989 | 1.00 | 31.27 | 6 | C |
| ATOM | 98 | C | ALA | A | 11 | 21.159 | 41.204 | −0.030 | 1.00 | 34.04 | 6 | C |
| ATOM | 99 | O | ALA | A | 11 | 21.684 | 42.248 | 0.371 | 1.00 | 31.26 | 8 | O |
| ATOM | 100 | CB | ALA | A | 11 | 19.493 | 40.259 | 1.531 | 1.00 | 29.04 | 6 | C |
| ATOM | 101 | N | ARG | A | 12 | 20.754 | 40.944 | −1.300 | 1.00 | 30.79 | 7 | N |
| ATOM | 102 | CA | ARG | A | 12 | 20.920 | 41.941 | −2.323 | 1.00 | 33.27 | 6 | C |
| ATOM | 103 | C | ARG | A | 12 | 19.990 | 43.076 | −1.933 | 1.00 | 32.96 | 6 | C |
| ATOM | 104 | O | ARG | A | 12 | 20.347 | 44.253 | −2.024 | 1.00 | 33.10 | 8 | O |
| ATOM | 105 | CB | ARG | A | 12 | 20.594 | 41.530 | −3.765 | 1.00 | 36.05 | 6 | C |
| ATOM | 106 | CG | ARG | A | 12 | 21.187 | 40.228 | −4.223 | 1.00 | 39.17 | 6 | C |
| ATOM | 107 | CD | ARG | A | 12 | 21.123 | 39.985 | −5.710 | 0.00 | 25.00 | 6 | C |
| ATOM | 108 | NE | ARG | A | 12 | 22.090 | 38.994 | −6.168 | 0.00 | 25.00 | 7 | N |
| ATOM | 109 | CZ | ARG | A | 12 | 23.073 | 39.249 | −7.020 | 0.00 | 25.00 | 6 | C |
| ATOM | 110 | NH1 | ARG | A | 12 | 23.207 | 40.464 | −7.531 | 0.00 | 25.00 | 7 | N |
| ATOM | 111 | NH2 | ARG | A | 12 | 23.921 | 38.288 | −7.356 | 0.00 | 25.00 | 7 | N |
| ATOM | 112 | N | SER | A | 13 | 18.819 | 42.783 | −1.322 | 1.00 | 32.93 | 7 | N |
| ATOM | 113 | CA | SER | A | 13 | 18.020 | 43.921 | −0.857 | 1.00 | 33.79 | 6 | C |
| ATOM | 114 | C | SER | A | 13 | 18.785 | 44.852 | 0.040 | 1.00 | 39.59 | 6 | C |
| ATOM | 115 | O | SER | A | 13 | 18.374 | 46.016 | 0.228 | 1.00 | 42.25 | 8 | O |
| ATOM | 116 | CB | SER | A | 13 | 16.700 | 43.412 | −0.328 | 1.00 | 32.35 | 6 | C |
| ATOM | 117 | OG | SER | A | 13 | 16.791 | 42.748 | 0.948 | 1.00 | 35.17 | 8 | O |
| ATOM | 118 | N | LEU | A | 14 | 19.912 | 44.484 | 0.697 | 1.00 | 43.32 | 7 | N |
| ATOM | 119 | CA | LEU | A | 14 | 20.676 | 45.332 | 1.583 | 1.00 | 45.46 | 6 | C |
| ATOM | 120 | C | LEU | A | 14 | 20.788 | 46.738 | 0.996 | 1.00 | 47.96 | 6 | C |
| ATOM | 121 | O | LEU | A | 14 | 20.716 | 47.698 | 1.692 | 1.00 | 44.53 | 8 | O |
| ATOM | 122 | CB | LEU | A | 14 | 22.096 | 44.793 | 1.759 | 1.00 | 46.15 | 6 | C |
| ATOM | 123 | CG | LEU | A | 14 | 22.962 | 45.090 | 2.949 | 1.00 | 45.96 | 6 | C |
| ATOM | 124 | CD1 | LEU | A | 14 | 24.020 | 46.102 | 2.591 | 1.00 | 47.65 | 6 | C |
| ATOM | 125 | CD2 | LEU | A | 14 | 22.107 | 45.655 | 4.071 | 1.00 | 48.57 | 6 | C |
| ATOM | 126 | N | VAL | A | 15 | 21.070 | 46.859 | −0.280 | 1.00 | 54.67 | 7 | N |
| ATOM | 127 | CA | VAL | A | 15 | 21.169 | 48.153 | −0.966 | 1.00 | 61.86 | 6 | C |
| ATOM | 128 | C | VAL | A | 15 | 19.771 | 48.721 | −0.944 | 1.00 | 67.28 | 6 | C |
| ATOM | 129 | O | VAL | A | 15 | 19.556 | 49.441 | 0.081 | 1.00 | 70.79 | 8 | O |
| ATOM | 130 | CB | VAL | A | 15 | 21.678 | 47.891 | −2.381 | 1.00 | 60.80 | 6 | C |
| ATOM | 131 | CG1 | VAL | A | 15 | 21.929 | 49.294 | −2.919 | 1.00 | 63.45 | 6 | C |
| ATOM | 132 | CG2 | VAL | A | 15 | 22.912 | 47.035 | −2.279 | 1.00 | 58.69 | 6 | C |
| ATOM | 133 | N | ASP | A | 16 | 18.842 | 48.423 | −1.873 | 1.00 | 69.66 | 7 | N |
| ATOM | 134 | CA | ASP | A | 16 | 17.492 | 48.905 | −1.592 | 1.00 | 73.65 | 6 | C |
| ATOM | 135 | C | ASP | A | 16 | 16.855 | 50.146 | −2.118 | 1.00 | 73.96 | 6 | C |
| ATOM | 136 | O | ASP | A | 16 | 15.621 | 50.250 | −1.876 | 1.00 | 75.09 | 8 | O |
| ATOM | 137 | CB | ASP | A | 16 | 17.256 | 49.122 | −0.054 | 1.00 | 76.53 | 6 | C |
| ATOM | 138 | CG | ASP | A | 16 | 15.860 | 48.741 | 0.394 | 1.00 | 82.22 | 6 | C |
| ATOM | 139 | OD1 | ASP | A | 16 | 15.092 | 47.968 | −0.241 | 1.00 | 82.82 | 8 | O |
| ATOM | 140 | OD2 | ASP | A | 16 | 15.546 | 49.286 | 1.479 | 1.00 | 85.01 | 8 | O |
| ATOM | 141 | N | GLY | A | 17 | 17.524 | 51.084 | −2.769 | 1.00 | 74.31 | 7 | N |
| ATOM | 142 | CA | GLY | A | 17 | 16.623 | 52.176 | −3.163 | 1.00 | 74.96 | 6 | C |
| ATOM | 143 | C | GLY | A | 17 | 17.373 | 53.481 | −3.185 | 1.00 | 75.85 | 6 | C |
| ATOM | 144 | O | GLY | A | 17 | 18.517 | 53.424 | −3.636 | 1.00 | 77.64 | 8 | O |
| ATOM | 145 | N | LEU | A | 18 | 16.650 | 54.502 | −2.715 | 1.00 | 73.64 | 7 | N |
| ATOM | 146 | CA | LEU | A | 18 | 17.295 | 55.813 | −2.774 | 1.00 | 70.08 | 6 | C |
| ATOM | 147 | C | LEU | A | 18 | 18.278 | 56.027 | −1.638 | 1.00 | 68.32 | 6 | C |
| ATOM | 148 | O | LEU | A | 18 | 17.945 | 55.924 | −0.463 | 1.00 | 69.03 | 8 | O |
| ATOM | 149 | CB | LEU | A | 18 | 16.151 | 56.795 | −2.820 | 1.00 | 70.56 | 6 | C |
| ATOM | 150 | CG | LEU | A | 18 | 14.841 | 56.681 | −3.579 | 1.00 | 69.33 | 6 | C |
| ATOM | 151 | CD1 | LEU | A | 18 | 13.995 | 57.916 | −3.217 | 1.00 | 68.34 | 6 | C |
| ATOM | 152 | CD2 | LEU | A | 18 | 14.911 | 56.617 | −5.082 | 1.00 | 66.83 | 6 | C |
| ATOM | 153 | N | THR | A | 19 | 19.512 | 56.365 | −1.988 | 1.00 | 66.52 | 7 | N |
| ATOM | 154 | CA | THR | A | 19 | 20.546 | 56.643 | −1.005 | 1.00 | 65.07 | 6 | C |
| ATOM | 155 | C | THR | A | 19 | 19.902 | 57.295 | 0.227 | 1.00 | 66.10 | 6 | C |
| ATOM | 156 | O | THR | A | 19 | 19.108 | 58.245 | 0.141 | 1.00 | 65.68 | 8 | O |
| ATOM | 157 | CB | THR | A | 19 | 21.677 | 57.462 | −1.604 | 1.00 | 63.90 | 6 | C |
| ATOM | 158 | OG1 | THR | A | 19 | 21.561 | 58.804 | −1.217 | 1.00 | 63.50 | 8 | O |
| ATOM | 159 | CG2 | THR | A | 19 | 21.645 | 57.367 | −3.143 | 1.00 | 65.35 | 6 | C |
| ATOM | 160 | N | SER | A | 20 | 20.194 | 56.707 | 1.398 | 1.00 | 64.66 | 7 | N |
| ATOM | 161 | CA | SER | A | 20 | 19.593 | 57.211 | 2.630 | 1.00 | 65.02 | 6 | C |
| ATOM | 162 | C | SER | A | 20 | 20.526 | 57.963 | 3.551 | 1.00 | 62.89 | 6 | C |
| ATOM | 163 | O | SER | A | 20 | 20.022 | 58.437 | 4.554 | 1.00 | 61.99 | 8 | O |
| ATOM | 164 | CB | SER | A | 20 | 18.856 | 56.060 | 3.322 | 1.00 | 67.58 | 6 | C |
| ATOM | 165 | OG | SER | A | 20 | 19.502 | 54.832 | 2.929 | 1.00 | 71.93 | 8 | O |
| ATOM | 166 | N | SER | A | 21 | 21.805 | 58.160 | 3.316 | 1.00 | 61.78 | 7 | N |
| ATOM | 167 | CA | SER | A | 21 | 22.680 | 58.957 | 4.149 | 1.00 | 61.68 | 6 | C |
| ATOM | 168 | C | SER | A | 21 | 23.575 | 59.691 | 3.137 | 1.00 | 59.24 | 6 | C |
| ATOM | 169 | O | SER | A | 21 | 23.748 | 59.187 | 2.029 | 1.00 | 59.16 | 8 | O |
| ATOM | 170 | CB | SER | A | 21 | 23.803 | 58.300 | 5.015 | 1.00 | 62.18 | 6 | C |
| ATOM | 171 | OG | SER | A | 21 | 23.699 | 58.475 | 6.401 | 0.00 | 30.00 | 8 | O |
| ATOM | 172 | N | HIS | A | 22 | 24.231 | 60.710 | 3.644 | 1.00 | 57.13 | 7 | N |
| ATOM | 173 | CA | HIS | A | 22 | 25.178 | 61.396 | 2.806 | 1.00 | 58.09 | 6 | C |
| ATOM | 174 | C | HIS | A | 22 | 26.417 | 60.508 | 2.747 | 1.00 | 57.37 | 6 | C |
| ATOM | 175 | O | HIS | A | 22 | 27.368 | 60.885 | 2.076 | 1.00 | 58.77 | 8 | O |
| ATOM | 176 | CB | HIS | A | 22 | 25.613 | 62.720 | 3.444 | 1.00 | 62.64 | 6 | C |
| ATOM | 177 | CG | HIS | A | 22 | 26.243 | 62.418 | 4.781 | 1.00 | 64.55 | 6 | C |
| ATOM | 178 | ND1 | HIS | A | 22 | 25.477 | 61.778 | 5.724 | 1.00 | 67.21 | 7 | N |
| ATOM | 179 | CD2 | HIS | A | 22 | 27.442 | 62.619 | 5.340 | 1.00 | 65.40 | 6 | C |
| ATOM | 180 | CE1 | HIS | A | 22 | 26.184 | 61.588 | 6.840 | 1.00 | 68.44 | 6 | C |
| ATOM | 181 | NE2 | HIS | A | 22 | 27.388 | 62.098 | 6.620 | 1.00 | 67.27 | 7 | N |
| ATOM | 182 | N | LEU | A | 23 | 26.460 | 59.352 | 3.424 | 1.00 | 54.28 | 7 | N |
| ATOM | 183 | CA | LEU | A | 23 | 27.606 | 58.477 | 3.365 | 1.00 | 51.08 | 6 | C |
| ATOM | 184 | C | LEU | A | 23 | 27.502 | 57.730 | 2.053 | 1.00 | 50.64 | 6 | C |
| ATOM | 185 | O | LEU | A | 23 | 28.485 | 57.104 | 1.620 | 1.00 | 55.30 | 8 | O |
| ATOM | 186 | CB | LEU | A | 23 | 27.656 | 57.566 | 4.586 | 1.00 | 50.86 | 6 | C |
| ATOM | 187 | CG | LEU | A | 23 | 28.116 | 58.228 | 5.867 | 1.00 | 48.97 | 6 | C |
| ATOM | 188 | CD1 | LEU | A | 23 | 27.806 | 57.396 | 7.080 | 1.00 | 49.29 | 6 | C |
| ATOM | 189 | CD2 | LEU | A | 23 | 29.624 | 58.488 | 5.764 | 1.00 | 51.85 | 6 | C |
| ATOM | 190 | N | GLY | A | 24 | 26.338 | 57.804 | 1.443 | 1.00 | 47.10 | 7 | N |
| ATOM | 191 | CA | GLY | A | 24 | 26.130 | 57.152 | 0.164 | 1.00 | 49.42 | 6 | C |
| ATOM | 192 | C | GLY | A | 24 | 25.393 | 55.820 | 0.294 | 1.00 | 48.86 | 6 | C |
| ATOM | 193 | O | GLY | A | 24 | 24.814 | 55.434 | 1.316 | 1.00 | 48.17 | 8 | O |
| ATOM | 194 | N | LYS | A | 25 | 25.447 | 55.071 | −0.811 | 1.00 | 49.55 | 7 | N |
| ATOM | 195 | CA | LYS | A | 25 | 24.755 | 53.757 | −0.780 | 1.00 | 50.51 | 6 | C |
| ATOM | 196 | C | LYS | A | 25 | 25.776 | 52.638 | −0.834 | 1.00 | 46.78 | 6 | C |
| ATOM | 197 | O | LYS | A | 25 | 26.871 | 52.803 | −1.377 | 1.00 | 48.22 | 8 | O |
| ATOM | 198 | CB | LYS | A | 25 | 23.841 | 53.546 | −2.007 | 1.00 | 55.99 | 6 | C |
| ATOM | 199 | CG | LYS | A | 25 | 22.786 | 52.855 | −1.747 | 0.00 | 25.00 | 6 | C |
| ATOM | 200 | CD | LYS | A | 25 | 22.261 | 52.228 | −3.001 | 0.00 | 25.00 | 6 | C |
| ATOM | 201 | CE | LYS | A | 25 | 20.808 | 52.581 | −3.183 | 0.00 | 25.00 | 6 | C |
| ATOM | 202 | NZ | LYS | A | 25 | 20.345 | 52.281 | −4.556 | 0.00 | 25.00 | 7 | N |
| ATOM | 203 | N | PRO | A | 26 | 25.395 | 51.533 | −0.210 | 1.00 | 42.68 | 7 | N |
| ATOM | 204 | CA | PRO | A | 26 | 26.320 | 50.416 | −0.166 | 1.00 | 39.07 | 6 | C |
| ATOM | 205 | C | PRO | A | 26 | 26.345 | 49.728 | −1.500 | 1.00 | 37.59 | 6 | C |
| ATOM | 206 | O | PRO | A | 26 | 25.274 | 49.603 | −2.053 | 1.00 | 39.43 | 8 | O |
| ATOM | 207 | CB | PRO | A | 26 | 25.588 | 49.529 | 0.799 | 1.00 | 39.98 | 6 | C |
| ATOM | 208 | CG | PRO | A | 26 | 24.547 | 50.256 | 1.531 | 1.00 | 37.68 | 6 | C |
| ATOM | 209 | CD | PRO | A | 26 | 24.158 | 51.252 | 0.517 | 1.00 | 40.06 | 6 | C |
| ATOM | 210 | N | ASP | A | 27 | 27.420 | 49.247 | −2.009 | 1.00 | 35.64 | 7 | N |
| ATOM | 211 | CA | ASP | A | 27 | 27.545 | 48.466 | −3.201 | 1.00 | 37.19 | 6 | C |
| ATOM | 212 | C | ASP | A | 27 | 27.456 | 47.022 | −2.677 | 1.00 | 38.07 | 6 | C |
| ATOM | 213 | O | ASP | A | 27 | 28.207 | 46.505 | −1.869 | 1.00 | 39.12 | 8 | O |
| ATOM | 214 | CB | ASP | A | 27 | 28.864 | 48.815 | −3.935 | 1.00 | 39.81 | 6 | C |
| ATOM | 215 | CG | ASP | A | 27 | 29.506 | 47.686 | −4.668 | 1.00 | 45.42 | 6 | C |
| ATOM | 216 | OD1 | ASP | A | 27 | 28.889 | 46.758 | −5.224 | 1.00 | 44.60 | 8 | O |
| ATOM | 217 | OD2 | ASP | A | 27 | 30.776 | 47.651 | −4.731 | 1.00 | 51.68 | 8 | O |
| ATOM | 218 | N | TYR | A | 28 | 26.500 | 46.248 | −3.116 | 1.00 | 38.37 | 7 | N |
| ATOM | 219 | CA | TYR | A | 28 | 26.290 | 44.874 | −2.769 | 1.00 | 39.05 | 6 | C |
| ATOM | 220 | C | TYR | A | 28 | 27.506 | 43.992 | −2.995 | 1.00 | 39.20 | 6 | C |
| ATOM | 221 | O | TYR | A | 28 | 27.885 | 43.196 | −2.109 | 1.00 | 39.63 | 8 | O |
| ATOM | 222 | CB | TYR | A | 28 | 25.161 | 44.248 | −3.554 | 1.00 | 37.78 | 6 | C |
| ATOM | 223 | CG | TYR | A | 28 | 24.883 | 42.820 | −3.190 | 1.00 | 43.82 | 6 | C |
| ATOM | 224 | CD1 | TYR | A | 28 | 24.630 | 42.441 | −1.848 | 1.00 | 43.26 | 6 | C |
| ATOM | 225 | CD2 | TYR | A | 28 | 24.855 | 41.814 | −4.145 | 1.00 | 43.70 | 6 | C |
| ATOM | 226 | CE1 | TYR | A | 28 | 24.370 | 41.150 | −1.494 | 1.00 | 43.70 | 6 | C |
| ATOM | 227 | CE2 | TYR | A | 28 | 24.593 | 40.497 | −3.794 | 1.00 | 45.14 | 6 | C |
| ATOM | 228 | CZ | TYR | A | 28 | 24.353 | 40.176 | −2.475 | 1.00 | 46.75 | 6 | C |
| ATOM | 229 | OH | TYR | A | 28 | 24.088 | 38.852 | −2.127 | 1.00 | 49.65 | 8 | O |
| ATOM | 230 | N | ALA | A | 29 | 28.118 | 44.137 | −4.154 | 1.00 | 38.45 | 7 | N |
| ATOM | 231 | CA | ALA | A | 29 | 29.293 | 43.320 | −4.471 | 1.00 | 36.75 | 6 | C |
| ATOM | 232 | C | ALA | A | 29 | 30.397 | 43.636 | −3.484 | 1.00 | 37.07 | 6 | C |
| ATOM | 233 | O | ALA | A | 29 | 30.906 | 42.611 | −3.013 | 1.00 | 40.68 | 8 | O |
| ATOM | 234 | CB | ALA | A | 29 | 29.675 | 43.419 | −5.951 | 1.00 | 35.51 | 6 | C |
| ATOM | 235 | N | LYS | A | 30 | 30.724 | 44.873 | −3.132 | 1.00 | 36.07 | 7 | N |
| ATOM | 236 | CA | LYS | A | 30 | 31.769 | 45.125 | −2.179 | 1.00 | 35.27 | 6 | C |
| ATOM | 237 | C | LYS | A | 30 | 31.412 | 44.610 | −0.791 | 1.00 | 35.07 | 6 | C |
| ATOM | 238 | O | LYS | A | 30 | 32.260 | 44.103 | −0.018 | 1.00 | 36.78 | 8 | O |
| ATOM | 239 | CB | LYS | A | 30 | 32.210 | 46.627 | −2.194 | 1.00 | 33.83 | 6 | C |
| ATOM | 240 | CG | LYS | A | 30 | 33.512 | 46.994 | −1.388 | 0.00 | 25.00 | 6 | C |
| ATOM | 241 | CD | LYS | A | 30 | 33.478 | 48.478 | −1.025 | 0.00 | 25.00 | 6 | C |
| ATOM | 242 | CE | LYS | A | 30 | 34.867 | 49.107 | −1.015 | 0.00 | 25.00 | 6 | C |
| ATOM | 243 | NZ | LYS | A | 30 | 34.812 | 50.554 | −0.646 | 0.00 | 25.00 | 7 | N |
| ATOM | 244 | N | ALA | A | 31 | 30.151 | 44.794 | −0.461 | 1.00 | 31.95 | 7 | N |
| ATOM | 245 | CA | ALA | A | 31 | 29.601 | 44.424 | 0.839 | 1.00 | 31.96 | 6 | C |
| ATOM | 246 | C | ALA | A | 31 | 29.650 | 42.892 | 0.961 | 1.00 | 31.51 | 6 | C |
| ATOM | 247 | O | ALA | A | 31 | 29.888 | 42.490 | 2.085 | 1.00 | 31.06 | 8 | O |
| ATOM | 248 | CB | ALA | A | 31 | 28.214 | 44.966 | 1.100 | 1.00 | 27.97 | 6 | C |
| ATOM | 249 | N | LEU | A | 32 | 29.459 | 42.206 | −0.135 | 1.00 | 32.78 | 7 | N |
| ATOM | 250 | CA | LEU | A | 32 | 29.569 | 40.723 | −0.129 | 1.00 | 33.84 | 6 | C |
| ATOM | 251 | C | LEU | A | 32 | 31.019 | 40.336 | 0.193 | 1.00 | 30.39 | 6 | C |
| ATOM | 252 | O | LEU | A | 32 | 31.167 | 39.560 | 1.144 | 1.00 | 28.93 | 8 | O |
| ATOM | 253 | CB | LEU | A | 32 | 28.998 | 40.056 | −1.364 | 1.00 | 32.99 | 6 | C |
| ATOM | 254 | CG | LEU | A | 32 | 28.619 | 38.587 | −1.260 | 1.00 | 35.14 | 6 | C |
| ATOM | 255 | CD1 | LEU | A | 32 | 27.326 | 38.356 | −0.487 | 1.00 | 32.70 | 6 | C |
| ATOM | 256 | CD2 | LEU | A | 32 | 28.525 | 37.877 | −2.598 | 1.00 | 32.76 | 6 | C |
| ATOM | 257 | N | GLU | A | 33 | 32.027 | 40.967 | −0.413 | 1.00 | 29.36 | 7 | N |
| ATOM | 258 | CA | GLU | A | 33 | 33.427 | 40.684 | −0.066 | 1.00 | 27.36 | 6 | C |
| ATOM | 259 | C | GLU | A | 33 | 33.765 | 41.061 | 1.353 | 1.00 | 26.68 | 6 | C |
| ATOM | 260 | O | GLU | A | 33 | 34.516 | 40.325 | 2.076 | 1.00 | 28.43 | 8 | O |
| ATOM | 261 | CB | GLU | A | 33 | 34.342 | 41.482 | −1.022 | 1.00 | 30.91 | 6 | C |
| ATOM | 262 | CG | GLU | A | 33 | 34.295 | 41.095 | −2.505 | 1.00 | 36.30 | 6 | C |
| ATOM | 263 | CD | GLU | A | 33 | 34.571 | 39.589 | −2.719 | 1.00 | 39.53 | 6 | C |
| ATOM | 264 | OE1 | GLU | A | 33 | 35.544 | 39.077 | −2.093 | 1.00 | 41.03 | 8 | O |
| ATOM | 265 | OE2 | GLU | A | 33 | 33.855 | 38.898 | −3.482 | 1.00 | 38.49 | 8 | O |
| ATOM | 266 | N | GLN | A | 34 | 33.222 | 42.195 | 1.834 | 1.00 | 24.37 | 7 | N |
| ATOM | 267 | CA | GLN | A | 34 | 33.634 | 42.548 | 3.212 | 1.00 | 24.50 | 6 | C |
| ATOM | 268 | C | GLN | A | 34 | 33.089 | 41.578 | 4.208 | 1.00 | 22.24 | 6 | C |
| ATOM | 269 | O | GLN | A | 34 | 33.615 | 41.228 | 5.187 | 1.00 | 20.90 | 8 | O |
| ATOM | 270 | CB | GLN | A | 34 | 33.231 | 43.990 | 3.516 | 1.00 | 29.92 | 6 | C |
| ATOM | 271 | CG | GLN | A | 34 | 33.547 | 44.925 | 2.344 | 1.00 | 31.30 | 6 | C |
| ATOM | 272 | CD | GLN | A | 34 | 33.268 | 46.357 | 2.828 | 1.00 | 34.98 | 6 | C |
| ATOM | 273 | OE1 | GLN | A | 34 | 32.247 | 46.581 | 3.474 | 1.00 | 31.81 | 8 | O |
| ATOM | 274 | NE2 | GLN | A | 34 | 34.210 | 47.253 | 2.482 | 1.00 | 38.64 | 7 | N |
| ATOM | 275 | N | HIS | A | 35 | 31.892 | 41.017 | 3.975 | 1.00 | 26.42 | 7 | N |
| ATOM | 276 | CA | HIS | A | 35 | 31.321 | 40.014 | 4.886 | 1.00 | 24.26 | 6 | C |
| ATOM | 277 | C | HIS | A | 35 | 32.137 | 38.700 | 4.891 | 1.00 | 24.90 | 6 | C |
| ATOM | 278 | O | HIS | A | 35 | 32.294 | 38.019 | 5.940 | 1.00 | 26.43 | 8 | O |
| ATOM | 279 | CB | HIS | A | 35 | 29.935 | 39.705 | 4.474 | 1.00 | 19.66 | 6 | C |
| ATOM | 280 | CG | HIS | A | 35 | 29.174 | 38.939 | 5.525 | 1.00 | 19.66 | 6 | C |
| ATOM | 281 | ND1 | HIS | A | 35 | 28.586 | 39.491 | 6.663 | 1.00 | 23.22 | 7 | N |
| ATOM | 282 | CD2 | HIS | A | 35 | 28.907 | 37.656 | 5.590 | 1.00 | 16.13 | 6 | C |
| ATOM | 283 | CE1 | HIS | A | 35 | 27.999 | 38.556 | 7.362 | 1.00 | 19.13 | 6 | C |
| ATOM | 284 | NE2 | HIS | A | 35 | 28.210 | 37.408 | 6.669 | 1.00 | 19.06 | 7 | N |
| ATOM | 285 | N | ASN | A | 36 | 32.619 | 38.387 | 3.706 | 1.00 | 22.39 | 7 | N |
| ATOM | 286 | CA | ASN | A | 36 | 33.459 | 37.246 | 3.425 | 1.00 | 25.00 | 6 | C |
| ATOM | 287 | C | ASN | A | 36 | 34.767 | 37.520 | 4.147 | 1.00 | 25.39 | 6 | C |
| ATOM | 288 | O | ASN | A | 36 | 35.217 | 36.692 | 4.941 | 1.00 | 23.90 | 8 | O |
| ATOM | 289 | CB | ASN | A | 36 | 33.509 | 36.969 | 1.903 | 1.00 | 30.86 | 6 | C |
| ATOM | 290 | CG | ASN | A | 36 | 34.376 | 35.698 | 1.802 | 1.00 | 36.31 | 6 | C |
| ATOM | 291 | OD1 | ASN | A | 36 | 33.942 | 34.697 | 2.429 | 1.00 | 42.74 | 8 | O |
| ATOM | 292 | ND2 | ASN | A | 36 | 35.487 | 35.636 | 1.128 | 1.00 | 34.95 | 7 | N |
| ATOM | 293 | N | ALA | A | 37 | 35.340 | 38.726 | 4.082 | 1.00 | 27.26 | 7 | N |
| ATOM | 294 | CA | ALA | A | 37 | 36.576 | 38.991 | 4.893 | 1.00 | 27.51 | 6 | C |
| ATOM | 295 | C | ALA | A | 37 | 36.270 | 38.906 | 6.365 | 1.00 | 26.90 | 6 | C |
| ATOM | 296 | O | ALA | A | 37 | 37.158 | 38.436 | 7.137 | 1.00 | 31.57 | 8 | O |
| ATOM | 297 | CB | ALA | A | 37 | 37.315 | 40.313 | 4.538 | 1.00 | 20.67 | 6 | C |
| ATOM | 298 | N | TYR | A | 38 | 35.037 | 39.243 | 6.763 | 1.00 | 25.18 | 7 | N |
| ATOM | 299 | CA | TYR | A | 38 | 34.700 | 39.165 | 8.192 | 1.00 | 24.23 | 6 | C |
| ATOM | 300 | C | TYR | A | 38 | 34.626 | 37.717 | 8.671 | 1.00 | 25.05 | 6 | C |
| ATOM | 301 | O | TYR | A | 38 | 34.994 | 37.434 | 9.818 | 1.00 | 24.85 | 8 | O |
| ATOM | 302 | CB | TYR | A | 38 | 33.301 | 39.805 | 8.345 | 1.00 | 21.85 | 6 | C |
| ATOM | 303 | CG | TYR | A | 38 | 32.635 | 39.904 | 9.700 | 1.00 | 21.49 | 6 | C |
| ATOM | 304 | CD1 | TYR | A | 38 | 33.161 | 40.779 | 10.718 | 1.00 | 22.78 | 6 | C |
| ATOM | 305 | CD2 | TYR | A | 38 | 31.530 | 39.185 | 10.007 | 1.00 | 19.03 | 6 | C |
| ATOM | 306 | CE1 | TYR | A | 38 | 32.521 | 40.850 | 11.953 | 1.00 | 22.09 | 6 | C |
| ATOM | 307 | CE2 | TYR | A | 38 | 30.895 | 39.272 | 11.274 | 1.00 | 22.37 | 6 | C |
| ATOM | 308 | CZ | TYR | A | 38 | 31.435 | 40.139 | 12.245 | 1.00 | 19.79 | 6 | C |
| ATOM | 309 | OH | TYR | A | 38 | 30.794 | 40.198 | 13.459 | 1.00 | 24.81 | 8 | O |
| ATOM | 310 | N | ILE | A | 39 | 34.010 | 36.817 | 7.874 | 1.00 | 24.91 | 7 | N |
| ATOM | 311 | CA | ILE | A | 39 | 33.890 | 35.378 | 8.217 | 1.00 | 24.75 | 6 | C |
| ATOM | 312 | C | ILE | A | 39 | 35.299 | 34.824 | 8.290 | 1.00 | 26.52 | 6 | C |
| ATOM | 313 | O | ILE | A | 39 | 35.680 | 34.219 | 9.311 | 1.00 | 27.63 | 8 | O |
| ATOM | 314 | CB | ILE | A | 39 | 32.953 | 34.577 | 7.319 | 1.00 | 25.52 | 6 | C |
| ATOM | 315 | CG1 | ILE | A | 39 | 31.526 | 35.142 | 7.344 | 1.00 | 20.50 | 6 | C |
| ATOM | 316 | CG2 | ILE | A | 39 | 32.852 | 33.062 | 7.550 | 1.00 | 23.99 | 6 | C |
| ATOM | 317 | CD1 | ILE | A | 39 | 30.963 | 35.229 | 8.795 | 1.00 | 18.38 | 6 | C |
| ATOM | 318 | N | ARG | A | 40 | 36.182 | 35.113 | 7.346 | 1.00 | 26.60 | 7 | N |
| ATOM | 319 | CA | ARG | A | 40 | 37.585 | 34.640 | 7.440 | 1.00 | 25.37 | 6 | C |
| ATOM | 320 | C | ARG | A | 40 | 38.293 | 35.124 | 8.655 | 1.00 | 29.74 | 6 | C |
| ATOM | 321 | O | ARG | A | 40 | 39.078 | 34.325 | 9.252 | 1.00 | 34.08 | 8 | O |
| ATOM | 322 | CB | ARG | A | 40 | 38.383 | 35.051 | 6.188 | 1.00 | 22.95 | 6 | C |
| ATOM | 323 | CG | ARG | A | 40 | 37.635 | 34.265 | 5.029 | 1.00 | 22.33 | 6 | C |
| ATOM | 324 | CD | ARG | A | 40 | 38.201 | 34.598 | 3.648 | 1.00 | 22.04 | 6 | C |
| ATOM | 325 | NE | ARG | A | 40 | 39.322 | 33.696 | 3.499 | 1.00 | 24.22 | 7 | N |
| ATOM | 326 | CZ | ARG | A | 40 | 40.161 | 33.452 | 2.557 | 1.00 | 28.66 | 6 | C |
| ATOM | 327 | NH1 | ARG | A | 40 | 39.921 | 34.235 | 1.476 | 1.00 | 35.84 | 7 | N |
| ATOM | 328 | NH2 | ARG | A | 40 | 41.144 | 32.532 | 2.656 | 1.00 | 28.13 | 7 | N |
| ATOM | 329 | N | ALA | A | 41 | 38.066 | 36.414 | 9.057 | 1.00 | 28.06 | 7 | N |
| ATOM | 330 | CA | ALA | A | 41 | 38.789 | 36.730 | 10.332 | 1.00 | 24.37 | 6 | C |
| ATOM | 331 | C | ALA | A | 41 | 38.211 | 35.884 | 11.419 | 1.00 | 27.29 | 6 | C |
| ATOM | 332 | O | ALA | A | 41 | 38.981 | 35.368 | 12.235 | 1.00 | 34.11 | 8 | O |
| ATOM | 333 | CB | ALA | A | 41 | 38.661 | 38.192 | 10.589 | 1.00 | 21.19 | 6 | C |
| ATOM | 334 | N | LEU | A | 42 | 36.880 | 35.712 | 11.486 | 1.00 | 25.91 | 7 | N |
| ATOM | 335 | CA | LEU | A | 42 | 36.330 | 34.941 | 12.577 | 1.00 | 26.34 | 6 | C |
| ATOM | 336 | C | LEU | A | 42 | 36.847 | 33.531 | 12.559 | 1.00 | 29.26 | 6 | C |
| ATOM | 337 | O | LEU | A | 42 | 37.177 | 32.927 | 13.577 | 1.00 | 34.17 | 8 | O |
| ATOM | 338 | CB | LEU | A | 42 | 34.804 | 35.006 | 12.455 | 1.00 | 24.15 | 6 | C |
| ATOM | 339 | CG | LEU | A | 42 | 34.234 | 36.390 | 12.903 | 1.00 | 25.87 | 6 | C |
| ATOM | 340 | CD1 | LEU | A | 42 | 32.756 | 36.586 | 12.400 | 1.00 | 24.15 | 6 | C |
| ATOM | 341 | CD2 | LEU | A | 42 | 34.408 | 36.600 | 14.359 | 1.00 | 18.61 | 6 | C |
| ATOM | 342 | N | GLN | A | 43 | 37.005 | 32.834 | 11.476 | 1.00 | 30.44 | 7 | N |
| ATOM | 343 | CA | GLN | A | 43 | 37.531 | 31.513 | 11.284 | 1.00 | 32.34 | 6 | C |
| ATOM | 344 | C | GLN | A | 43 | 38.868 | 31.338 | 11.982 | 1.00 | 34.70 | 6 | C |
| ATOM | 345 | O | GLN | A | 43 | 39.280 | 30.241 | 12.267 | 1.00 | 37.30 | 8 | O |
| ATOM | 346 | CB | GLN | A | 43 | 37.685 | 31.240 | 9.777 | 1.00 | 30.76 | 6 | C |
| ATOM | 347 | CG | GLN | A | 43 | 36.423 | 30.706 | 9.193 | 1.00 | 30.88 | 6 | C |
| ATOM | 348 | CD | GLN | A | 43 | 36.618 | 30.485 | 7.705 | 1.00 | 32.57 | 6 | C |
| ATOM | 349 | OE1 | GLN | A | 43 | 36.771 | 29.314 | 7.310 | 1.00 | 35.75 | 8 | O |
| ATOM | 350 | NE2 | GLN | A | 43 | 36.621 | 31.494 | 6.878 | 1.00 | 28.89 | 7 | N |
| ATOM | 351 | N | THR | A | 44 | 39.625 | 32.363 | 12.270 | 1.00 | 35.85 | 7 | N |
| ATOM | 352 | CA | THR | A | 44 | 40.874 | 32.334 | 13.040 | 1.00 | 36.77 | 6 | C |
| ATOM | 353 | C | THR | A | 44 | 40.596 | 32.444 | 14.503 | 1.00 | 36.18 | 6 | C |
| ATOM | 354 | O | THR | A | 44 | 41.463 | 32.091 | 15.258 | 1.00 | 39.71 | 8 | O |
| ATOM | 355 | CB | THR | A | 44 | 41.475 | 33.594 | 12.385 | 1.00 | 38.69 | 6 | C |
| ATOM | 356 | OG1 | THR | A | 44 | 42.219 | 33.125 | 11.253 | 1.00 | 38.83 | 8 | O |
| ATOM | 357 | CG2 | THR | A | 44 | 42.108 | 34.565 | 13.297 | 1.00 | 41.01 | 6 | C |
| ATOM | 358 | N | CYS | A | 45 | 39.486 | 32.826 | 15.098 | 1.00 | 34.29 | 7 | N |
| ATOM | 359 | CA | CYS | A | 45 | 39.262 | 32.976 | 16.528 | 1.00 | 34.07 | 6 | C |
| ATOM | 360 | C | CYS | A | 45 | 38.895 | 31.770 | 17.346 | 1.00 | 35.08 | 6 | C |
| ATOM | 361 | O | CYS | A | 45 | 38.372 | 31.905 | 18.469 | 1.00 | 37.73 | 8 | O |
| ATOM | 362 | CB | CYS | A | 45 | 38.172 | 34.104 | 16.630 | 1.00 | 29.98 | 6 | C |
| ATOM | 363 | SG | CYS | A | 45 | 38.733 | 35.572 | 15.712 | 1.00 | 35.91 | 16 | S |
| ATOM | 364 | N | ASP | A | 46 | 39.156 | 30.549 | 16.939 | 1.00 | 34.71 | 7 | N |
| ATOM | 365 | CA | ASP | A | 46 | 38.871 | 29.321 | 17.661 | 1.00 | 35.36 | 6 | C |
| ATOM | 366 | C | ASP | A | 46 | 37.423 | 29.288 | 18.109 | 1.00 | 33.28 | 6 | C |
| ATOM | 367 | O | ASP | A | 46 | 37.030 | 29.221 | 19.240 | 1.00 | 30.74 | 8 | O |
| ATOM | 368 | CB | ASP | A | 46 | 39.725 | 29.147 | 18.913 | 1.00 | 43.62 | 6 | C |
| ATOM | 369 | CG | ASP | A | 46 | 41.210 | 29.107 | 18.615 | 1.00 | 53.54 | 6 | C |
| ATOM | 370 | OD1 | ASP | A | 46 | 41.667 | 27.985 | 18.258 | 1.00 | 57.92 | 8 | O |
| ATOM | 371 | OD2 | ASP | A | 46 | 41.909 | 30.164 | 18.731 | 1.00 | 57.53 | 8 | O |
| ATOM | 372 | N | VAL | A | 47 | 36.513 | 29.370 | 17.184 | 1.00 | 32.70 | 7 | N |
| ATOM | 373 | CA | VAL | A | 47 | 35.070 | 29.383 | 17.424 | 1.00 | 29.52 | 6 | C |
| ATOM | 374 | C | VAL | A | 47 | 34.492 | 28.729 | 16.239 | 1.00 | 28.10 | 6 | C |
| ATOM | 375 | O | VAL | A | 47 | 35.166 | 28.715 | 15.152 | 1.00 | 29.55 | 8 | O |
| ATOM | 376 | CB | VAL | A | 47 | 34.833 | 30.912 | 17.555 | 1.00 | 33.41 | 6 | C |
| ATOM | 377 | CG1 | VAL | A | 47 | 34.380 | 31.531 | 16.254 | 1.00 | 29.25 | 6 | C |
| ATOM | 378 | CG2 | VAL | A | 47 | 34.007 | 31.245 | 18.801 | 1.00 | 33.11 | 6 | C |
| ATOM | 379 | N | ASP | A | 48 | 33.343 | 28.104 | 16.395 | 1.00 | 27.21 | 7 | N |
| ATOM | 380 | CA | ASP | A | 48 | 32.774 | 27.580 | 15.137 | 1.00 | 28.52 | 6 | C |
| ATOM | 381 | C | ASP | A | 48 | 31.769 | 28.656 | 14.700 | 1.00 | 26.68 | 6 | C |
| ATOM | 382 | O | ASP | A | 48 | 31.218 | 29.457 | 15.487 | 1.00 | 25.75 | 8 | O |
| ATOM | 383 | CB | ASP | A | 48 | 32.223 | 26.210 | 15.169 | 1.00 | 33.17 | 6 | C |
| ATOM | 384 | CG | ASP | A | 48 | 31.303 | 25.949 | 16.287 | 1.00 | 37.40 | 6 | C |
| ATOM | 385 | OD1 | ASP | A | 48 | 30.661 | 26.975 | 16.604 | 1.00 | 39.92 | 8 | O |
| ATOM | 386 | OD2 | ASP | A | 48 | 31.210 | 24.818 | 16.825 | 1.00 | 41.59 | 8 | O |
| ATOM | 387 | N | ILE | A | 49 | 31.421 | 28.693 | 13.459 | 1.00 | 24.70 | 7 | N |
| ATOM | 388 | CA | ILE | A | 49 | 30.563 | 29.648 | 12.852 | 1.00 | 25.72 | 6 | C |
| ATOM | 389 | C | ILE | A | 49 | 29.294 | 29.073 | 12.308 | 1.00 | 30.12 | 6 | C |
| ATOM | 390 | O | ILE | A | 49 | 29.420 | 28.144 | 11.491 | 1.00 | 35.34 | 8 | O |
| ATOM | 391 | CB | ILE | A | 49 | 31.383 | 30.196 | 11.675 | 1.00 | 25.39 | 6 | C |
| ATOM | 392 | CG1 | ILE | A | 49 | 32.551 | 31.010 | 12.334 | 1.00 | 21.19 | 6 | C |
| ATOM | 393 | CG2 | ILE | A | 49 | 30.593 | 31.039 | 10.713 | 1.00 | 25.74 | 6 | C |
| ATOM | 394 | CD1 | ILE | A | 49 | 33.519 | 31.605 | 11.415 | 1.00 | 20.51 | 6 | C |
| ATOM | 395 | N | THR | A | 50 | 28.102 | 29.590 | 12.616 | 1.00 | 27.07 | 7 | N |
| ATOM | 396 | CA | THR | A | 50 | 26.904 | 29.167 | 11.983 | 1.00 | 23.13 | 6 | C |
| ATOM | 397 | C | THR | A | 50 | 26.636 | 30.260 | 10.921 | 1.00 | 25.15 | 6 | C |
| ATOM | 398 | O | THR | A | 50 | 26.468 | 31.382 | 11.444 | 1.00 | 28.58 | 8 | O |
| ATOM | 399 | CB | THR | A | 50 | 25.876 | 29.007 | 13.008 | 1.00 | 21.68 | 6 | C |
| ATOM | 400 | OG1 | THR | A | 50 | 26.374 | 27.840 | 13.652 | 1.00 | 27.10 | 8 | O |
| ATOM | 401 | CG2 | THR | A | 50 | 24.512 | 28.613 | 12.406 | 1.00 | 22.71 | 6 | C |
| ATOM | 402 | N | LEU | A | 51 | 26.758 | 30.078 | 9.646 | 1.00 | 20.93 | 7 | N |
| ATOM | 403 | CA | LEU | A | 51 | 26.555 | 31.164 | 8.633 | 1.00 | 20.68 | 6 | C |
| ATOM | 404 | C | LEU | A | 51 | 25.206 | 30.985 | 7.990 | 1.00 | 23.53 | 6 | C |
| ATOM | 405 | O | LEU | A | 51 | 24.907 | 29.912 | 7.333 | 1.00 | 26.60 | 8 | O |
| ATOM | 406 | CB | LEU | A | 51 | 27.515 | 31.105 | 7.466 | 1.00 | 18.66 | 6 | C |
| ATOM | 407 | CG | LEU | A | 51 | 27.808 | 32.163 | 6.448 | 1.00 | 23.76 | 6 | C |
| ATOM | 408 | CD1 | LEU | A | 51 | 27.634 | 33.633 | 6.985 | 1.00 | 25.13 | 6 | C |
| ATOM | 409 | CD2 | LEU | A | 51 | 29.272 | 32.111 | 5.953 | 1.00 | 18.53 | 6 | C |
| ATOM | 410 | N | LEU | A | 52 | 24.264 | 31.863 | 8.260 | 1.00 | 22.18 | 7 | N |
| ATOM | 411 | CA | LEU | A | 52 | 22.944 | 31.643 | 7.687 | 1.00 | 20.34 | 6 | C |
| ATOM | 412 | C | LEU | A | 52 | 22.813 | 32.323 | 6.304 | 1.00 | 18.64 | 6 | C |
| ATOM | 413 | O | LEU | A | 52 | 23.523 | 33.209 | 5.981 | 1.00 | 16.87 | 8 | O |
| ATOM | 414 | CB | LEU | A | 52 | 21.913 | 32.266 | 8.646 | 1.00 | 22.30 | 6 | C |
| ATOM | 415 | CG | LEU | A | 52 | 21.869 | 31.698 | 10.069 | 1.00 | 25.14 | 6 | C |
| ATOM | 416 | CD1 | LEU | A | 52 | 20.888 | 32.486 | 10.925 | 1.00 | 20.85 | 6 | C |
| ATOM | 417 | CD2 | LEU | A | 52 | 21.698 | 30.196 | 10.156 | 1.00 | 19.64 | 6 | C |
| ATOM | 418 | N | PRO | A | 53 | 21.862 | 31.869 | 5.487 | 1.00 | 19.01 | 7 | N |
| ATOM | 419 | CA | PRO | A | 53 | 21.541 | 32.307 | 4.134 | 1.00 | 21.41 | 6 | C |
| ATOM | 420 | C | PRO | A | 53 | 20.860 | 33.676 | 4.099 | 1.00 | 24.30 | 6 | C |
| ATOM | 421 | O | PRO | A | 53 | 20.235 | 34.099 | 5.066 | 1.00 | 24.47 | 8 | O |
| ATOM | 422 | CB | PRO | A | 53 | 20.596 | 31.209 | 3.559 | 1.00 | 16.73 | 6 | C |
| ATOM | 423 | CG | PRO | A | 53 | 19.898 | 30.852 | 4.922 | 1.00 | 18.80 | 6 | C |
| ATOM | 424 | CD | PRO | A | 53 | 20.980 | 30.688 | 5.927 | 1.00 | 14.11 | 6 | C |
| ATOM | 425 | N | PRO | A | 54 | 21.057 | 34.395 | 2.991 | 1.00 | 27.10 | 7 | N |
| ATOM | 426 | CA | PRO | A | 54 | 20.486 | 35.714 | 2.805 | 1.00 | 28.32 | 6 | C |
| ATOM | 427 | C | PRO | A | 54 | 18.958 | 35.494 | 2.774 | 1.00 | 33.08 | 6 | C |
| ATOM | 428 | O | PRO | A | 54 | 18.427 | 34.440 | 2.454 | 1.00 | 33.86 | 8 | O |
| ATOM | 429 | CB | PRO | A | 54 | 20.982 | 36.164 | 1.477 | 1.00 | 26.61 | 6 | C |
| ATOM | 430 | CG | PRO | A | 54 | 21.568 | 34.994 | 0.750 | 1.00 | 24.83 | 6 | C |
| ATOM | 431 | CD | PRO | A | 54 | 21.743 | 33.904 | 1.752 | 1.00 | 26.69 | 6 | C |
| ATOM | 432 | N | ASP | A | 55 | 18.268 | 36.487 | 3.265 | 1.00 | 35.16 | 7 | N |
| ATOM | 433 | CA | ASP | A | 55 | 16.823 | 36.534 | 3.287 | 1.00 | 34.29 | 6 | C |
| ATOM | 434 | C | ASP | A | 55 | 16.507 | 37.924 | 2.692 | 1.00 | 36.97 | 6 | C |
| ATOM | 435 | O | ASP | A | 55 | 16.690 | 38.977 | 3.307 | 1.00 | 38.84 | 8 | O |
| ATOM | 436 | CB | ASP | A | 55 | 16.302 | 36.328 | 4.666 | 1.00 | 30.78 | 6 | C |
| ATOM | 437 | CG | ASP | A | 55 | 14.861 | 35.884 | 4.624 | 1.00 | 31.36 | 6 | C |
| ATOM | 438 | OD1 | ASP | A | 55 | 14.110 | 36.272 | 3.724 | 1.00 | 27.22 | 8 | O |
| ATOM | 439 | OD2 | ASP | A | 55 | 14.436 | 35.104 | 5.533 | 1.00 | 35.57 | 8 | O |
| ATOM | 440 | N | GLU | A | 56 | 16.123 | 37.953 | 1.432 | 1.00 | 36.78 | 7 | N |
| ATOM | 441 | CA | GLU | A | 56 | 15.791 | 39.112 | 0.617 | 1.00 | 37.87 | 6 | C |
| ATOM | 442 | C | GLU | A | 56 | 14.580 | 39.853 | 1.197 | 1.00 | 38.05 | 6 | C |
| ATOM | 443 | O | GLU | A | 56 | 14.430 | 41.039 | 0.910 | 1.00 | 37.55 | 8 | O |
| ATOM | 444 | CB | GLU | A | 56 | 15.498 | 38.777 | −0.858 | 1.00 | 35.35 | 6 | C |
| ATOM | 445 | CG | GLU | A | 56 | 16.795 | 38.184 | −1.450 | 1.00 | 35.32 | 6 | C |
| ATOM | 446 | CD | GLU | A | 56 | 17.818 | 39.281 | −1.544 | 1.00 | 38.08 | 6 | C |
| ATOM | 447 | OE1 | GLU | A | 56 | 17.541 | 40.468 | −1.313 | 1.00 | 38.12 | 8 | O |
| ATOM | 448 | OE2 | GLU | A | 56 | 18.990 | 39.066 | −1.860 | 1.00 | 43.09 | 8 | O |
| ATOM | 449 | N | ARG | A | 57 | 13.843 | 39.171 | 2.045 | 1.00 | 35.05 | 7 | N |
| ATOM | 450 | CA | ARG | A | 57 | 12.724 | 39.843 | 2.681 | 1.00 | 35.86 | 6 | C |
| ATOM | 451 | C | ARG | A | 57 | 13.212 | 40.797 | 3.768 | 1.00 | 36.89 | 6 | C |
| ATOM | 452 | O | ARG | A | 57 | 12.455 | 41.624 | 4.194 | 1.00 | 33.57 | 8 | O |
| ATOM | 453 | CB | ARG | A | 57 | 11.888 | 38.743 | 3.347 | 1.00 | 35.40 | 6 | C |
| ATOM | 454 | CG | ARG | A | 57 | 10.912 | 38.015 | 2.428 | 1.00 | 35.86 | 6 | C |
| ATOM | 455 | CD | ARG | A | 57 | 10.280 | 36.867 | 3.207 | 1.00 | 41.11 | 6 | C |
| ATOM | 456 | NE | ARG | A | 57 | 11.270 | 36.029 | 3.844 | 1.00 | 43.62 | 7 | N |
| ATOM | 457 | CZ | ARG | A | 57 | 10.953 | 35.017 | 4.665 | 1.00 | 46.02 | 6 | C |
| ATOM | 458 | NH1 | ARG | A | 57 | 9.696 | 34.728 | 4.934 | 1.00 | 42.67 | 7 | N |
| ATOM | 459 | NH2 | ARG | A | 57 | 11.938 | 34.273 | 5.231 | 1.00 | 43.71 | 7 | N |
| ATOM | 460 | N | PHE | A | 58 | 14.438 | 40.663 | 4.314 | 1.00 | 35.40 | 7 | N |
| ATOM | 461 | CA | PHE | A | 58 | 15.008 | 41.468 | 5.356 | 1.00 | 31.50 | 6 | C |
| ATOM | 462 | C | PHE | A | 58 | 16.387 | 42.033 | 5.068 | 1.00 | 32.43 | 6 | C |
| ATOM | 463 | O | PHE | A | 58 | 17.450 | 41.521 | 5.488 | 1.00 | 33.72 | 8 | O |
| ATOM | 464 | CB | PHE | A | 58 | 15.074 | 40.705 | 6.680 | 1.00 | 31.73 | 6 | C |
| ATOM | 465 | CG | PHE | A | 58 | 13.701 | 40.229 | 7.072 | 1.00 | 33.55 | 6 | C |
| ATOM | 466 | CD1 | PHE | A | 58 | 13.300 | 38.954 | 6.778 | 1.00 | 36.83 | 6 | C |
| ATOM | 467 | CD2 | PHE | A | 58 | 12.814 | 41.074 | 7.728 | 1.00 | 33.80 | 6 | C |
| ATOM | 468 | CE1 | PHE | A | 58 | 12.020 | 38.538 | 7.155 | 1.00 | 38.15 | 6 | C |
| ATOM | 469 | CE2 | PHE | A | 58 | 11.565 | 40.597 | 8.067 | 1.00 | 35.99 | 6 | C |
| ATOM | 470 | CZ | PHE | A | 58 | 11.107 | 39.324 | 7.801 | 1.00 | 32.90 | 6 | C |
| ATOM | 471 | N | PRO | A | 59 | 16.410 | 43.217 | 4.454 | 1.00 | 31.86 | 7 | N |
| ATOM | 472 | CA | PRO | A | 59 | 17.581 | 43.912 | 4.059 | 1.00 | 30.00 | 6 | C |
| ATOM | 473 | C | PRO | A | 59 | 18.589 | 44.017 | 5.140 | 1.00 | 32.05 | 6 | C |
| ATOM | 474 | O | PRO | A | 59 | 19.772 | 44.013 | 4.860 | 1.00 | 33.61 | 8 | O |
| ATOM | 475 | CB | PRO | A | 59 | 17.044 | 45.334 | 3.666 | 1.00 | 30.27 | 6 | C |
| ATOM | 476 | CG | PRO | A | 59 | 15.663 | 45.043 | 3.097 | 1.00 | 29.24 | 6 | C |
| ATOM | 477 | CD | PRO | A | 59 | 15.138 | 43.887 | 3.908 | 1.00 | 32.56 | 6 | C |
| ATOM | 478 | N | ASP | A | 60 | 18.215 | 44.221 | 6.378 | 1.00 | 31.89 | 7 | N |
| ATOM | 479 | CA | ASP | A | 60 | 19.159 | 44.372 | 7.490 | 1.00 | 32.41 | 6 | C |
| ATOM | 480 | C | ASP | A | 60 | 19.373 | 43.034 | 8.180 | 1.00 | 30.02 | 6 | C |
| ATOM | 481 | O | ASP | A | 60 | 20.027 | 43.030 | 9.222 | 1.00 | 28.97 | 8 | O |
| ATOM | 482 | CB | ASP | A | 60 | 18.473 | 45.419 | 8.447 | 1.00 | 33.85 | 6 | C |
| ATOM | 483 | CG | ASP | A | 60 | 18.483 | 46.809 | 7.842 | 1.00 | 37.89 | 6 | C |
| ATOM | 484 | OD1 | ASP | A | 60 | 19.584 | 47.413 | 7.628 | 1.00 | 40.07 | 8 | O |
| ATOM | 485 | OD2 | ASP | A | 60 | 17.477 | 47.484 | 7.504 | 1.00 | 37.08 | 8 | O |
| ATOM | 486 | N | SER | A | 61 | 18.901 | 41.884 | 7.663 | 1.00 | 27.23 | 7 | N |
| ATOM | 487 | CA | SER | A | 61 | 19.108 | 40.675 | 8.457 | 1.00 | 28.36 | 6 | C |
| ATOM | 488 | C | SER | A | 61 | 20.571 | 40.358 | 8.543 | 1.00 | 25.73 | 6 | C |
| ATOM | 489 | O | SER | A | 61 | 20.863 | 39.528 | 9.372 | 1.00 | 26.88 | 8 | O |
| ATOM | 490 | CB | SER | A | 61 | 18.446 | 39.326 | 8.002 | 1.00 | 30.66 | 6 | C |
| ATOM | 491 | OG | SER | A | 61 | 18.346 | 39.409 | 6.583 | 1.00 | 31.07 | 8 | O |
| ATOM | 492 | N | VAL | A | 62 | 21.477 | 40.957 | 7.796 | 1.00 | 25.63 | 7 | N |
| ATOM | 493 | CA | VAL | A | 62 | 22.886 | 40.693 | 7.991 | 1.00 | 24.54 | 6 | C |
| ATOM | 494 | C | VAL | A | 62 | 23.212 | 41.164 | 9.417 | 1.00 | 26.53 | 6 | C |
| ATOM | 495 | O | VAL | A | 62 | 24.216 | 40.638 | 9.917 | 1.00 | 27.73 | 8 | O |
| ATOM | 496 | CB | VAL | A | 62 | 23.692 | 41.490 | 6.961 | 1.00 | 24.13 | 6 | C |
| ATOM | 497 | CG1 | VAL | A | 62 | 23.411 | 43.008 | 7.227 | 1.00 | 23.39 | 6 | C |
| ATOM | 498 | CG2 | VAL | A | 62 | 25.158 | 41.192 | 7.192 | 1.00 | 21.16 | 6 | C |
| ATOM | 499 | N | PHE | A | 63 | 22.471 | 42.087 | 10.044 | 1.00 | 25.99 | 7 | N |
| ATOM | 500 | CA | PHE | A | 63 | 22.816 | 42.475 | 11.383 | 1.00 | 29.11 | 6 | C |
| ATOM | 501 | C | PHE | A | 63 | 22.198 | 41.513 | 12.409 | 1.00 | 30.42 | 6 | C |
| ATOM | 502 | O | PHE | A | 63 | 21.319 | 42.034 | 13.102 | 1.00 | 31.13 | 8 | O |
| ATOM | 503 | CB | PHE | A | 63 | 22.402 | 43.933 | 11.718 | 1.00 | 27.93 | 6 | C |
| ATOM | 504 | CG | PHE | A | 63 | 23.108 | 44.821 | 10.712 | 1.00 | 30.27 | 6 | C |
| ATOM | 505 | CD1 | PHE | A | 63 | 22.362 | 45.629 | 9.886 | 1.00 | 30.49 | 6 | C |
| ATOM | 506 | CD2 | PHE | A | 63 | 24.484 | 44.846 | 10.591 | 1.00 | 29.21 | 6 | C |
| ATOM | 507 | CE1 | PHE | A | 63 | 23.043 | 46.425 | 8.973 | 1.00 | 32.10 | 6 | C |
| ATOM | 508 | CE2 | PHE | A | 63 | 25.101 | 45.646 | 9.687 | 1.00 | 28.92 | 6 | C |
| ATOM | 509 | CZ | PHE | A | 63 | 24.399 | 46.453 | 8.857 | 1.00 | 30.37 | 6 | C |
| ATOM | 510 | N | VAL | A | 64 | 22.737 | 40.319 | 12.539 | 1.00 | 27.88 | 7 | N |
| ATOM | 511 | CA | VAL | A | 64 | 22.146 | 39.359 | 13.474 | 1.00 | 28.28 | 6 | C |
| ATOM | 512 | C | VAL | A | 64 | 22.247 | 39.792 | 14.898 | 1.00 | 27.87 | 6 | C |
| ATOM | 513 | O | VAL | A | 64 | 21.486 | 39.271 | 15.721 | 1.00 | 27.81 | 8 | O |
| ATOM | 514 | CB | VAL | A | 64 | 22.650 | 37.897 | 13.246 | 1.00 | 27.99 | 6 | C |
| ATOM | 515 | CG1 | VAL | A | 64 | 22.457 | 37.544 | 11.757 | 1.00 | 28.02 | 6 | C |
| ATOM | 516 | CG2 | VAL | A | 64 | 24.151 | 37.702 | 13.542 | 1.00 | 23.97 | 6 | C |
| ATOM | 517 | N | GLU | A | 65 | 23.149 | 40.688 | 15.298 | 1.00 | 28.38 | 7 | N |
| ATOM | 518 | CA | GLU | A | 65 | 23.162 | 41.104 | 16.702 | 1.00 | 26.08 | 6 | C |
| ATOM | 519 | C | GLU | A | 65 | 21.794 | 41.581 | 17.187 | 1.00 | 27.45 | 6 | C |
| ATOM | 520 | O | GLU | A | 65 | 21.359 | 41.282 | 18.311 | 1.00 | 28.15 | 8 | O |
| ATOM | 521 | CB | GLU | A | 65 | 24.117 | 42.274 | 16.802 | 1.00 | 26.92 | 6 | C |
| ATOM | 522 | CG | GLU | A | 65 | 24.126 | 42.976 | 18.166 | 1.00 | 28.58 | 6 | C |
| ATOM | 523 | CD | GLU | A | 65 | 25.444 | 43.678 | 18.345 | 1.00 | 29.06 | 6 | C |
| ATOM | 524 | OE1 | GLU | A | 65 | 25.436 | 44.798 | 17.749 | 1.00 | 33.68 | 8 | O |
| ATOM | 525 | OE2 | GLU | A | 65 | 26.445 | 43.278 | 18.963 | 1.00 | 32.32 | 8 | O |
| ATOM | 526 | N | ASP | A | 66 | 20.928 | 42.265 | 16.482 | 1.00 | 27.02 | 7 | N |
| ATOM | 527 | CA | ASP | A | 66 | 19.685 | 42.889 | 16.884 | 1.00 | 27.62 | 6 | C |
| ATOM | 528 | C | ASP | A | 66 | 18.592 | 42.019 | 17.391 | 1.00 | 29.94 | 6 | C |
| ATOM | 529 | O | ASP | A | 66 | 18.132 | 42.178 | 18.504 | 1.00 | 32.85 | 8 | O |
| ATOM | 530 | CB | ASP | A | 66 | 19.270 | 43.962 | 15.878 | 1.00 | 26.56 | 6 | C |
| ATOM | 531 | CG | ASP | A | 66 | 20.317 | 45.071 | 16.092 | 1.00 | 31.25 | 6 | C |
| ATOM | 532 | OD1 | ASP | A | 66 | 20.776 | 45.423 | 17.242 | 1.00 | 31.97 | 8 | O |
| ATOM | 533 | OD2 | ASP | A | 66 | 20.701 | 45.606 | 15.037 | 1.00 | 33.10 | 8 | O |
| ATOM | 534 | N | PRO | A | 67 | 18.252 | 40.968 | 16.679 | 1.00 | 31.77 | 7 | N |
| ATOM | 535 | CA | PRO | A | 67 | 17.241 | 40.053 | 17.099 | 1.00 | 29.37 | 6 | C |
| ATOM | 536 | C | PRO | A | 67 | 17.448 | 39.096 | 18.233 | 1.00 | 31.41 | 6 | C |
| ATOM | 537 | O | PRO | A | 67 | 16.420 | 38.428 | 18.510 | 1.00 | 33.18 | 8 | O |
| ATOM | 538 | CB | PRO | A | 67 | 17.007 | 39.275 | 15.820 | 1.00 | 29.86 | 6 | C |
| ATOM | 539 | CG | PRO | A | 67 | 18.124 | 39.550 | 14.879 | 1.00 | 30.41 | 6 | C |
| ATOM | 540 | CD | PRO | A | 67 | 18.796 | 40.796 | 15.288 | 1.00 | 28.88 | 6 | C |
| ATOM | 541 | N | VAL | A | 68 | 18.545 | 38.836 | 18.945 | 1.00 | 30.40 | 7 | N |
| ATOM | 542 | CA | VAL | A | 68 | 18.566 | 37.861 | 19.992 | 1.00 | 30.34 | 6 | C |
| ATOM | 543 | C | VAL | A | 68 | 19.749 | 38.300 | 20.924 | 1.00 | 32.47 | 6 | C |
| ATOM | 544 | O | VAL | A | 68 | 20.667 | 38.844 | 20.297 | 1.00 | 31.27 | 8 | O |
| ATOM | 545 | CB | VAL | A | 68 | 19.125 | 36.464 | 19.600 | 1.00 | 35.49 | 6 | C |
| ATOM | 546 | CG1 | VAL | A | 68 | 18.744 | 35.303 | 20.591 | 1.00 | 34.53 | 6 | C |
| ATOM | 547 | CG2 | VAL | A | 68 | 18.825 | 35.953 | 18.206 | 1.00 | 32.75 | 6 | C |
| ATOM | 548 | N | LEU | A | 69 | 19.707 | 38.056 | 22.238 | 1.00 | 31.41 | 7 | N |
| ATOM | 549 | CA | LEU | A | 69 | 20.848 | 38.340 | 23.076 | 1.00 | 31.10 | 6 | C |
| ATOM | 550 | C | LEU | A | 69 | 21.246 | 36.867 | 23.499 | 1.00 | 33.71 | 6 | C |
| ATOM | 551 | O | LEU | A | 69 | 20.281 | 36.154 | 23.947 | 1.00 | 35.34 | 8 | O |
| ATOM | 552 | CB | LEU | A | 69 | 20.493 | 39.033 | 24.357 | 1.00 | 30.39 | 6 | C |
| ATOM | 553 | CG | LEU | A | 69 | 21.585 | 39.543 | 25.235 | 1.00 | 31.74 | 6 | C |
| ATOM | 554 | CD1 | LEU | A | 69 | 22.129 | 40.700 | 24.478 | 1.00 | 34.28 | 6 | C |
| ATOM | 555 | CD2 | LEU | A | 69 | 21.021 | 40.087 | 26.597 | 1.00 | 35.48 | 6 | C |
| ATOM | 556 | N | CYS | A | 70 | 22.512 | 36.453 | 23.382 | 1.00 | 28.50 | 7 | N |
| ATOM | 557 | CA | CYS | A | 70 | 22.892 | 35.110 | 23.763 | 1.00 | 31.35 | 6 | C |
| ATOM | 558 | C | CYS | A | 70 | 23.740 | 35.209 | 25.015 | 1.00 | 33.80 | 6 | C |
| ATOM | 559 | O | CYS | A | 70 | 24.825 | 35.818 | 25.027 | 1.00 | 34.34 | 8 | O |
| ATOM | 560 | CB | CYS | A | 70 | 23.669 | 34.344 | 22.642 | 1.00 | 30.28 | 6 | C |
| ATOM | 561 | SG | CYS | A | 70 | 22.537 | 34.093 | 21.181 | 1.00 | 31.35 | 16 | S |
| ATOM | 562 | N | THR | A | 71 | 23.191 | 34.599 | 26.079 | 1.00 | 37.46 | 7 | N |
| ATOM | 563 | CA | THR | A | 71 | 23.943 | 34.709 | 27.374 | 1.00 | 39.71 | 6 | C |
| ATOM | 564 | C | THR | A | 71 | 24.364 | 33.291 | 27.651 | 1.00 | 41.48 | 6 | C |
| ATOM | 565 | O | THR | A | 71 | 23.934 | 32.399 | 26.862 | 1.00 | 40.94 | 8 | O |
| ATOM | 566 | CB | THR | A | 71 | 22.978 | 35.210 | 28.439 | 1.00 | 40.24 | 6 | C |
| ATOM | 567 | OG1 | THR | A | 71 | 21.963 | 34.199 | 28.561 | 1.00 | 41.56 | 8 | O |
| ATOM | 568 | CG2 | THR | A | 71 | 22.220 | 36.426 | 27.892 | 1.00 | 35.95 | 6 | C |
| ATOM | 569 | N | SER | A | 72 | 25.101 | 33.143 | 28.755 | 1.00 | 40.00 | 7 | N |
| ATOM | 570 | CA | SER | A | 72 | 25.581 | 31.806 | 29.099 | 1.00 | 36.31 | 6 | C |
| ATOM | 571 | C | SER | A | 72 | 24.528 | 30.976 | 29.745 | 1.00 | 39.27 | 6 | C |
| ATOM | 572 | O | SER | A | 72 | 24.862 | 29.901 | 30.231 | 1.00 | 42.43 | 8 | O |
| ATOM | 573 | CB | SER | A | 72 | 26.722 | 32.061 | 30.084 | 1.00 | 36.25 | 6 | C |
| ATOM | 574 | OG | SER | A | 72 | 26.050 | 32.650 | 31.246 | 1.00 | 38.53 | 8 | O |
| ATOM | 575 | N | ARG | A | 73 | 23.269 | 31.374 | 29.815 | 1.00 | 41.04 | 7 | N |
| ATOM | 576 | CA | ARG | A | 73 | 22.253 | 30.594 | 30.513 | 1.00 | 41.40 | 6 | C |
| ATOM | 577 | C | ARG | A | 73 | 21.007 | 30.606 | 29.636 | 1.00 | 41.34 | 6 | C |
| ATOM | 578 | O | ARG | A | 73 | 20.221 | 29.603 | 29.712 | 1.00 | 46.04 | 8 | O |
| ATOM | 579 | CB | ARG | A | 73 | 21.886 | 31.161 | 31.891 | 1.00 | 42.16 | 6 | C |
| ATOM | 580 | CG | ARG | A | 73 | 23.019 | 31.575 | 32.797 | 1.00 | 45.77 | 6 | C |
| ATOM | 581 | CD | ARG | A | 73 | 23.324 | 30.953 | 34.112 | 1.00 | 52.47 | 6 | C |
| ATOM | 582 | NE | ARG | A | 73 | 24.335 | 31.693 | 34.896 | 1.00 | 57.98 | 7 | N |
| ATOM | 583 | CZ | ARG | A | 73 | 24.030 | 32.484 | 35.924 | 1.00 | 60.40 | 6 | C |
| ATOM | 584 | NH1 | ARG | A | 73 | 22.736 | 32.598 | 36.246 | 1.00 | 61.61 | 7 | N |
| ATOM | 585 | NH2 | ARG | A | 73 | 24.892 | 33.178 | 36.673 | 1.00 | 61.75 | 7 | N |
| ATOM | 586 | N | CYS | A | 74 | 20.710 | 31.598 | 28.861 | 1.00 | 36.56 | 7 | N |
| ATOM | 587 | CA | CYS | A | 74 | 19.480 | 31.523 | 28.068 | 1.00 | 36.88 | 6 | C |
| ATOM | 588 | C | CYS | A | 74 | 19.781 | 32.340 | 26.813 | 1.00 | 36.05 | 6 | C |
| ATOM | 589 | O | CYS | A | 74 | 20.922 | 32.871 | 26.697 | 1.00 | 39.58 | 8 | O |
| ATOM | 590 | CB | CYS | A | 74 | 18.243 | 32.088 | 28.757 | 1.00 | 38.40 | 6 | C |
| ATOM | 591 | SG | CYS | A | 74 | 18.228 | 33.865 | 29.190 | 1.00 | 44.59 | 16 | S |
| ATOM | 592 | N | ALA | A | 75 | 18.826 | 32.448 | 25.950 | 1.00 | 33.14 | 7 | N |
| ATOM | 593 | CA | ALA | A | 75 | 18.936 | 33.349 | 24.802 | 1.00 | 34.63 | 6 | C |
| ATOM | 594 | C | ALA | A | 75 | 17.502 | 34.003 | 24.768 | 1.00 | 35.44 | 6 | C |
| ATOM | 595 | O | ALA | A | 75 | 16.518 | 33.291 | 24.962 | 1.00 | 34.93 | 8 | O |
| ATOM | 596 | CB | ALA | A | 75 | 19.081 | 32.821 | 23.392 | 1.00 | 30.52 | 6 | C |
| ATOM | 597 | N | ILE | A | 76 | 17.449 | 35.303 | 24.486 | 1.00 | 31.88 | 7 | N |
| ATOM | 598 | CA | ILE | A | 76 | 16.184 | 35.976 | 24.456 | 1.00 | 29.70 | 6 | C |
| ATOM | 599 | C | ILE | A | 76 | 15.913 | 36.491 | 23.063 | 1.00 | 31.68 | 6 | C |
| ATOM | 600 | O | ILE | A | 76 | 16.698 | 37.327 | 22.461 | 1.00 | 33.66 | 8 | O |
| ATOM | 601 | CB | ILE | A | 76 | 16.336 | 37.135 | 25.556 | 1.00 | 27.80 | 6 | C |
| ATOM | 602 | CG1 | ILE | A | 76 | 16.554 | 36.637 | 26.977 | 1.00 | 25.83 | 6 | C |
| ATOM | 603 | CG2 | ILE | A | 76 | 15.039 | 37.917 | 25.417 | 1.00 | 28.27 | 6 | C |
| ATOM | 604 | CD1 | ILE | A | 76 | 18.051 | 36.626 | 27.434 | 1.00 | 26.23 | 6 | C |
| ATOM | 605 | N | ILE | A | 77 | 14.858 | 36.049 | 22.385 | 1.00 | 30.90 | 7 | N |
| ATOM | 606 | CA | ILE | A | 77 | 14.575 | 36.590 | 21.050 | 1.00 | 30.11 | 6 | C |
| ATOM | 607 | C | ILE | A | 77 | 14.143 | 38.023 | 21.386 | 1.00 | 35.09 | 6 | C |
| ATOM | 608 | O | ILE | A | 77 | 13.316 | 38.193 | 22.333 | 1.00 | 40.24 | 8 | O |
| ATOM | 609 | CB | ILE | A | 77 | 13.415 | 35.862 | 20.399 | 1.00 | 31.67 | 6 | C |
| ATOM | 610 | CG1 | ILE | A | 77 | 13.730 | 34.348 | 20.277 | 1.00 | 36.56 | 6 | C |
| ATOM | 611 | CG2 | ILE | A | 77 | 13.104 | 36.323 | 19.020 | 1.00 | 30.14 | 6 | C |
| ATOM | 612 | CD1 | ILE | A | 77 | 14.871 | 34.067 | 19.291 | 1.00 | 35.01 | 6 | C |
| ATOM | 613 | N | THR | A | 78 | 14.594 | 39.079 | 20.731 | 1.00 | 34.47 | 7 | N |
| ATOM | 614 | CA | THR | A | 78 | 14.250 | 40.418 | 21.124 | 1.00 | 34.03 | 6 | C |
| ATOM | 615 | C | THR | A | 78 | 13.042 | 40.818 | 20.333 | 1.00 | 37.00 | 6 | C |
| ATOM | 616 | O | THR | A | 78 | 12.410 | 39.875 | 19.847 | 1.00 | 40.06 | 8 | O |
| ATOM | 617 | CB | THR | A | 78 | 15.445 | 41.352 | 20.839 | 1.00 | 34.73 | 6 | C |
| ATOM | 618 | OG1 | THR | A | 78 | 15.675 | 41.361 | 19.426 | 1.00 | 32.31 | 8 | O |
| ATOM | 619 | CG2 | THR | A | 78 | 16.626 | 40.878 | 21.655 | 1.00 | 33.05 | 6 | C |
| ATOM | 620 | N | ARG | A | 79 | 12.657 | 42.037 | 20.165 | 1.00 | 35.94 | 7 | N |
| ATOM | 621 | CA | ARG | A | 79 | 11.470 | 42.447 | 19.415 | 1.00 | 35.29 | 6 | C |
| ATOM | 622 | C | ARG | A | 79 | 11.799 | 43.911 | 18.998 | 1.00 | 36.78 | 6 | C |
| ATOM | 623 | O | ARG | A | 79 | 11.549 | 44.922 | 19.678 | 1.00 | 35.76 | 8 | O |
| ATOM | 624 | CB | ARG | A | 79 | 10.239 | 42.405 | 20.346 | 1.00 | 35.77 | 6 | C |
| ATOM | 625 | CG | ARG | A | 79 | 9.019 | 42.873 | 19.475 | 1.00 | 35.31 | 6 | C |
| ATOM | 626 | CD | ARG | A | 79 | 7.767 | 42.511 | 20.291 | 1.00 | 38.35 | 6 | C |
| ATOM | 627 | NE | ARG | A | 79 | 6.672 | 43.036 | 19.491 | 1.00 | 40.63 | 7 | N |
| ATOM | 628 | CZ | ARG | A | 79 | 6.259 | 44.297 | 19.463 | 1.00 | 40.32 | 6 | C |
| ATOM | 629 | NH1 | ARG | A | 79 | 6.881 | 45.176 | 20.222 | 1.00 | 36.52 | 7 | N |
| ATOM | 630 | NH2 | ARG | A | 79 | 5.223 | 44.527 | 18.634 | 1.00 | 41.78 | 7 | N |
| ATOM | 631 | N | PRO | A | 80 | 12.467 | 44.008 | 17.880 | 1.00 | 38.93 | 7 | N |
| ATOM | 632 | CA | PRO | A | 80 | 13.033 | 45.210 | 17.333 | 1.00 | 40.61 | 6 | C |
| ATOM | 633 | C | PRO | A | 80 | 12.051 | 46.342 | 17.159 | 1.00 | 39.05 | 6 | C |
| ATOM | 634 | O | PRO | A | 80 | 10.957 | 46.079 | 16.775 | 1.00 | 38.29 | 8 | O |
| ATOM | 635 | CB | PRO | A | 80 | 13.750 | 44.827 | 16.035 | 1.00 | 39.62 | 6 | C |
| ATOM | 636 | CG | PRO | A | 80 | 13.892 | 43.361 | 16.076 | 1.00 | 39.34 | 6 | C |
| ATOM | 637 | CD | PRO | A | 80 | 12.841 | 42.846 | 16.997 | 1.00 | 42.12 | 6 | C |
| ATOM | 638 | N | GLY | A | 81 | 12.531 | 47.519 | 17.455 | 1.00 | 39.77 | 7 | N |
| ATOM | 639 | CA | GLY | A | 81 | 11.646 | 48.721 | 17.314 | 1.00 | 42.41 | 6 | C |
| ATOM | 640 | C | GLY | A | 81 | 11.427 | 48.926 | 15.845 | 1.00 | 43.43 | 6 | C |
| ATOM | 641 | O | GLY | A | 81 | 10.312 | 49.167 | 15.466 | 1.00 | 45.38 | 8 | O |
| ATOM | 642 | N | ALA | A | 82 | 12.470 | 48.772 | 15.016 | 1.00 | 43.72 | 7 | N |
| ATOM | 643 | CA | ALA | A | 82 | 12.173 | 48.956 | 13.580 | 1.00 | 44.48 | 6 | C |
| ATOM | 644 | C | ALA | A | 82 | 11.273 | 47.818 | 13.146 | 1.00 | 47.68 | 6 | C |
| ATOM | 645 | O | ALA | A | 82 | 11.586 | 46.630 | 13.236 | 1.00 | 47.18 | 8 | O |
| ATOM | 646 | CB | ALA | A | 82 | 13.490 | 49.036 | 12.825 | 1.00 | 43.34 | 6 | C |
| ATOM | 647 | N | GLU | A | 83 | 10.118 | 48.087 | 12.596 | 1.00 | 49.49 | 7 | N |
| ATOM | 648 | CA | GLU | A | 83 | 9.143 | 47.143 | 12.091 | 1.00 | 49.64 | 6 | C |
| ATOM | 649 | C | GLU | A | 83 | 9.499 | 46.477 | 10.787 | 1.00 | 48.16 | 6 | C |
| ATOM | 650 | O | GLU | A | 83 | 8.974 | 45.518 | 10.209 | 1.00 | 49.66 | 8 | O |
| ATOM | 651 | CB | GLU | A | 83 | 7.964 | 48.151 | 12.044 | 1.00 | 53.64 | 6 | C |
| ATOM | 652 | CG | GLU | A | 83 | 6.701 | 47.375 | 11.869 | 1.00 | 59.06 | 6 | C |
| ATOM | 653 | CD | GLU | A | 83 | 5.460 | 48.049 | 12.414 | 1.00 | 62.18 | 6 | C |
| ATOM | 654 | OE1 | GLU | A | 83 | 5.534 | 49.082 | 13.106 | 1.00 | 62.90 | 8 | O |
| ATOM | 655 | OE2 | GLU | A | 83 | 4.448 | 47.385 | 12.033 | 1.00 | 63.18 | 8 | O |
| ATOM | 656 | N | SER | A | 84 | 10.562 | 46.983 | 10.170 | 1.00 | 46.07 | 7 | N |
| ATOM | 657 | CA | SER | A | 84 | 11.003 | 46.316 | 8.922 | 1.00 | 44.81 | 6 | C |
| ATOM | 658 | C | SER | A | 84 | 11.939 | 45.178 | 9.293 | 1.00 | 43.65 | 6 | C |
| ATOM | 659 | O | SER | A | 84 | 12.152 | 44.366 | 8.463 | 1.00 | 45.26 | 8 | O |
| ATOM | 660 | CB | SER | A | 84 | 11.668 | 47.313 | 8.002 | 1.00 | 43.66 | 6 | C |
| ATOM | 661 | OG | SER | A | 84 | 12.899 | 47.794 | 8.427 | 1.00 | 43.12 | 8 | O |
| ATOM | 662 | N | ARG | A | 85 | 12.488 | 45.127 | 10.482 | 1.00 | 42.86 | 7 | N |
| ATOM | 663 | CA | ARG | A | 85 | 13.381 | 44.159 | 11.014 | 1.00 | 40.83 | 6 | C |
| ATOM | 664 | C | ARG | A | 85 | 12.690 | 43.281 | 12.071 | 1.00 | 42.90 | 6 | C |
| ATOM | 665 | O | ARG | A | 85 | 13.337 | 42.399 | 12.628 | 1.00 | 43.73 | 8 | O |
| ATOM | 666 | CB | ARG | A | 85 | 14.579 | 44.866 | 11.679 | 1.00 | 35.86 | 6 | C |
| ATOM | 667 | CG | ARG | A | 85 | 15.290 | 45.945 | 10.891 | 1.00 | 30.73 | 6 | C |
| ATOM | 668 | CD | ARG | A | 85 | 16.770 | 46.158 | 11.316 | 1.00 | 27.47 | 6 | C |
| ATOM | 669 | NE | ARG | A | 85 | 17.367 | 44.829 | 11.518 | 1.00 | 27.90 | 7 | N |
| ATOM | 670 | CZ | ARG | A | 85 | 18.460 | 44.602 | 12.226 | 1.00 | 28.75 | 6 | C |
| ATOM | 671 | NH1 | ARG | A | 85 | 19.104 | 45.611 | 12.811 | 1.00 | 25.10 | 7 | N |
| ATOM | 672 | NH2 | ARG | A | 85 | 18.878 | 43.311 | 12.323 | 1.00 | 29.00 | 7 | N |
| ATOM | 673 | N | ARG | A | 86 | 11.412 | 43.441 | 12.351 | 1.00 | 42.27 | 7 | N |
| ATOM | 674 | CA | ARG | A | 86 | 10.793 | 42.613 | 13.391 | 1.00 | 43.53 | 6 | C |
| ATOM | 675 | C | ARG | A | 86 | 10.651 | 41.159 | 13.014 | 1.00 | 42.53 | 6 | C |
| ATOM | 676 | O | ARG | A | 86 | 10.894 | 40.317 | 13.902 | 1.00 | 41.77 | 8 | O |
| ATOM | 677 | CB | ARG | A | 86 | 9.478 | 43.233 | 13.904 | 1.00 | 45.21 | 6 | C |
| ATOM | 678 | CG | ARG | A | 86 | 8.998 | 42.836 | 15.269 | 1.00 | 45.81 | 6 | C |
| ATOM | 679 | CD | ARG | A | 86 | 7.966 | 43.769 | 15.870 | 1.00 | 50.08 | 6 | C |
| ATOM | 680 | NE | ARG | A | 86 | 8.533 | 45.042 | 16.316 | 1.00 | 52.09 | 7 | N |
| ATOM | 681 | CZ | ARG | A | 86 | 7.852 | 46.184 | 16.325 | 1.00 | 54.44 | 6 | C |
| ATOM | 682 | NH1 | ARG | A | 86 | 6.597 | 46.122 | 15.899 | 1.00 | 55.12 | 7 | N |
| ATOM | 683 | NH2 | ARG | A | 86 | 8.314 | 47.370 | 16.716 | 1.00 | 54.52 | 7 | N |
| ATOM | 684 | N | GLY | A | 87 | 10.306 | 40.753 | 11.814 | 1.00 | 41.30 | 7 | N |
| ATOM | 685 | CA | GLY | A | 87 | 10.214 | 39.368 | 11.411 | 1.00 | 38.40 | 6 | C |
| ATOM | 686 | C | GLY | A | 87 | 11.594 | 38.725 | 11.390 | 1.00 | 37.29 | 6 | C |
| ATOM | 687 | O | GLY | A | 87 | 11.646 | 37.528 | 11.374 | 1.00 | 37.00 | 8 | O |
| ATOM | 688 | N | GLU | A | 88 | 12.737 | 39.395 | 11.492 | 1.00 | 34.87 | 7 | N |
| ATOM | 689 | CA | GLU | A | 88 | 14.028 | 38.747 | 11.507 | 1.00 | 33.51 | 6 | C |
| ATOM | 690 | C | GLU | A | 88 | 14.070 | 37.846 | 12.725 | 1.00 | 36.42 | 6 | C |
| ATOM | 691 | O | GLU | A | 88 | 14.853 | 36.869 | 12.683 | 1.00 | 39.36 | 8 | O |
| ATOM | 692 | CB | GLU | A | 88 | 15.202 | 39.721 | 11.476 | 1.00 | 31.83 | 6 | C |
| ATOM | 693 | CG | GLU | A | 88 | 15.237 | 40.606 | 10.230 | 1.00 | 26.70 | 6 | C |
| ATOM | 694 | CD | GLU | A | 88 | 16.268 | 41.678 | 10.478 | 1.00 | 30.70 | 6 | C |
| ATOM | 695 | OE1 | GLU | A | 88 | 17.242 | 41.345 | 11.210 | 1.00 | 31.05 | 8 | O |
| ATOM | 696 | OE2 | GLU | A | 88 | 16.182 | 42.849 | 9.996 | 1.00 | 30.69 | 8 | O |
| ATOM | 697 | N | THR | A | 89 | 13.240 | 38.100 | 13.769 | 1.00 | 34.68 | 7 | N |
| ATOM | 698 | CA | THR | A | 89 | 13.225 | 37.183 | 14.882 | 1.00 | 35.28 | 6 | C |
| ATOM | 699 | C | THR | A | 89 | 12.697 | 35.837 | 14.444 | 1.00 | 35.41 | 6 | C |
| ATOM | 700 | O | THR | A | 89 | 13.190 | 34.803 | 14.881 | 1.00 | 38.37 | 8 | O |
| ATOM | 701 | CB | THR | A | 89 | 12.315 | 37.721 | 16.003 | 1.00 | 40.06 | 6 | C |
| ATOM | 702 | OG1 | THR | A | 89 | 11.000 | 37.923 | 15.417 | 1.00 | 42.92 | 8 | O |
| ATOM | 703 | CG2 | THR | A | 89 | 12.844 | 39.051 | 16.491 | 1.00 | 40.29 | 6 | C |
| ATOM | 704 | N | GLU | A | 90 | 11.741 | 35.652 | 13.589 | 1.00 | 34.81 | 7 | N |
| ATOM | 705 | CA | GLU | A | 90 | 11.225 | 34.363 | 13.233 | 1.00 | 39.76 | 6 | C |
| ATOM | 706 | C | GLU | A | 90 | 12.142 | 33.512 | 12.396 | 1.00 | 39.51 | 6 | C |
| ATOM | 707 | O | GLU | A | 90 | 11.909 | 32.323 | 12.437 | 1.00 | 40.76 | 8 | O |
| ATOM | 708 | CB | GLU | A | 90 | 9.875 | 34.461 | 12.518 | 1.00 | 44.99 | 6 | C |
| ATOM | 709 | CG | GLU | A | 90 | 8.826 | 35.215 | 13.362 | 1.00 | 55.34 | 6 | C |
| ATOM | 710 | CD | GLU | A | 90 | 7.485 | 35.264 | 12.648 | 1.00 | 59.16 | 6 | C |
| ATOM | 711 | OE1 | GLU | A | 90 | 7.639 | 35.121 | 11.403 | 1.00 | 61.89 | 8 | O |
| ATOM | 712 | OE2 | GLU | A | 90 | 6.444 | 35.428 | 13.331 | 1.00 | 61.11 | 8 | O |
| ATOM | 713 | N | ILE | A | 91 | 13.089 | 33.970 | 11.623 | 1.00 | 38.04 | 7 | N |
| ATOM | 714 | CA | ILE | A | 91 | 14.010 | 33.282 | 10.769 | 1.00 | 33.30 | 6 | C |
| ATOM | 715 | C | ILE | A | 91 | 15.337 | 33.072 | 11.508 | 1.00 | 34.96 | 6 | C |
| ATOM | 716 | O | ILE | A | 91 | 16.279 | 32.582 | 10.883 | 1.00 | 37.55 | 8 | O |
| ATOM | 717 | CB | ILE | A | 91 | 14.461 | 34.074 | 9.501 | 1.00 | 29.78 | 6 | C |
| ATOM | 718 | CG1 | ILE | A | 91 | 14.929 | 35.487 | 9.792 | 1.00 | 27.82 | 6 | C |
| ATOM | 719 | CG2 | ILE | A | 91 | 13.329 | 34.252 | 8.509 | 1.00 | 27.01 | 6 | C |
| ATOM | 720 | CD1 | ILE | A | 91 | 15.487 | 36.167 | 8.500 | 1.00 | 26.88 | 6 | C |
| ATOM | 721 | N | ILE | A | 92 | 15.504 | 33.451 | 12.773 | 1.00 | 31.69 | 7 | N |
| ATOM | 722 | CA | ILE | A | 92 | 16.737 | 33.126 | 13.420 | 1.00 | 29.83 | 6 | C |
| ATOM | 723 | C | ILE | A | 92 | 16.469 | 32.149 | 14.588 | 1.00 | 29.38 | 6 | C |
| ATOM | 724 | O | ILE | A | 92 | 17.417 | 31.545 | 15.161 | 1.00 | 32.63 | 8 | O |
| ATOM | 725 | CB | ILE | A | 92 | 17.417 | 34.418 | 13.954 | 1.00 | 29.20 | 6 | C |
| ATOM | 726 | CG1 | ILE | A | 92 | 18.886 | 34.099 | 14.253 | 1.00 | 30.18 | 6 | C |
| ATOM | 727 | CG2 | ILE | A | 92 | 16.696 | 34.887 | 15.184 | 1.00 | 29.20 | 6 | C |
| ATOM | 728 | CD1 | ILE | A | 92 | 19.833 | 35.226 | 14.382 | 1.00 | 30.71 | 6 | C |
| ATOM | 729 | N | GLU | A | 93 | 15.230 | 32.013 | 14.953 | 1.00 | 29.73 | 7 | N |
| ATOM | 730 | CA | GLU | A | 93 | 14.835 | 31.228 | 16.131 | 1.00 | 36.81 | 6 | C |
| ATOM | 731 | C | GLU | A | 93 | 15.119 | 29.738 | 16.071 | 1.00 | 35.23 | 6 | C |
| ATOM | 732 | O | GLU | A | 93 | 15.465 | 29.149 | 17.065 | 1.00 | 37.35 | 8 | O |
| ATOM | 733 | CB | GLU | A | 93 | 13.359 | 31.422 | 16.442 | 1.00 | 41.55 | 6 | C |
| ATOM | 734 | CG | GLU | A | 93 | 13.048 | 30.875 | 17.846 | 1.00 | 48.03 | 6 | C |
| ATOM | 735 | CD | GLU | A | 93 | 11.632 | 31.345 | 18.213 | 1.00 | 50.63 | 6 | C |
| ATOM | 736 | OE1 | GLU | A | 93 | 11.245 | 32.438 | 17.716 | 1.00 | 53.99 | 8 | O |
| ATOM | 737 | OE2 | GLU | A | 93 | 11.041 | 30.556 | 18.978 | 1.00 | 51.98 | 8 | O |
| ATOM | 738 | N | GLU | A | 94 | 14.996 | 29.174 | 14.903 | 1.00 | 35.33 | 7 | N |
| ATOM | 739 | CA | GLU | A | 94 | 15.297 | 27.766 | 14.775 | 1.00 | 38.81 | 6 | C |
| ATOM | 740 | C | GLU | A | 94 | 16.763 | 27.512 | 15.120 | 1.00 | 35.59 | 6 | C |
| ATOM | 741 | O | GLU | A | 94 | 16.956 | 26.483 | 15.774 | 1.00 | 35.01 | 8 | O |
| ATOM | 742 | CB | GLU | A | 94 | 14.829 | 27.314 | 13.405 | 1.00 | 43.28 | 6 | C |
| ATOM | 743 | CG | GLU | A | 94 | 15.015 | 25.766 | 13.291 | 1.00 | 51.17 | 6 | C |
| ATOM | 744 | CD | GLU | A | 94 | 14.677 | 25.506 | 11.825 | 1.00 | 59.39 | 6 | C |
| ATOM | 745 | OE1 | GLU | A | 94 | 13.478 | 25.861 | 11.536 | 1.00 | 63.20 | 8 | O |
| ATOM | 746 | OE2 | GLU | A | 94 | 15.621 | 24.993 | 11.122 | 1.00 | 60.88 | 8 | O |
| ATOM | 747 | N | THR | A | 95 | 17.733 | 28.277 | 14.763 | 1.00 | 31.81 | 7 | N |
| ATOM | 748 | CA | THR | A | 95 | 19.140 | 28.208 | 15.095 | 1.00 | 31.15 | 6 | C |
| ATOM | 749 | C | THR | A | 95 | 19.338 | 28.436 | 16.605 | 1.00 | 33.49 | 6 | C |
| ATOM | 750 | O | THR | A | 95 | 20.129 | 27.781 | 17.195 | 1.00 | 34.78 | 8 | O |
| ATOM | 751 | CB | THR | A | 95 | 19.838 | 29.418 | 14.413 | 1.00 | 29.04 | 6 | C |
| ATOM | 752 | OG1 | THR | A | 95 | 19.760 | 29.421 | 12.983 | 1.00 | 30.97 | 8 | O |
| ATOM | 753 | CG2 | THR | A | 95 | 21.298 | 29.662 | 14.678 | 1.00 | 25.39 | 6 | C |
| ATOM | 754 | N | VAL | A | 96 | 18.685 | 29.463 | 17.222 | 1.00 | 33.86 | 7 | N |
| ATOM | 755 | CA | VAL | A | 96 | 18.805 | 29.735 | 18.636 | 1.00 | 34.31 | 6 | C |
| ATOM | 756 | C | VAL | A | 96 | 18.238 | 28.541 | 19.407 | 1.00 | 33.71 | 6 | C |
| ATOM | 757 | O | VAL | A | 96 | 18.779 | 28.050 | 20.390 | 1.00 | 35.38 | 8 | O |
| ATOM | 758 | CB | VAL | A | 96 | 18.044 | 31.042 | 19.033 | 1.00 | 38.10 | 6 | C |
| ATOM | 759 | CG1 | VAL | A | 96 | 18.272 | 31.294 | 20.534 | 1.00 | 36.05 | 6 | C |
| ATOM | 760 | CG2 | VAL | A | 96 | 18.594 | 32.243 | 18.292 | 1.00 | 38.55 | 6 | C |
| ATOM | 761 | N | GLN | A | 97 | 17.132 | 28.008 | 19.034 | 1.00 | 35.46 | 7 | N |
| ATOM | 762 | CA | GLN | A | 97 | 16.490 | 26.836 | 19.706 | 1.00 | 39.88 | 6 | C |
| ATOM | 763 | C | GLN | A | 97 | 17.511 | 25.724 | 19.685 | 1.00 | 41.14 | 6 | C |
| ATOM | 764 | O | GLN | A | 97 | 17.895 | 25.107 | 20.697 | 1.00 | 43.77 | 8 | O |
| ATOM | 765 | CB | GLN | A | 97 | 15.201 | 26.607 | 18.960 | 1.00 | 43.60 | 6 | C |
| ATOM | 766 | CG | GLN | A | 97 | 13.866 | 26.203 | 19.571 | 1.00 | 46.07 | 6 | C |
| ATOM | 767 | CD | GLN | A | 97 | 13.459 | 27.128 | 20.702 | 1.00 | 49.58 | 6 | C |
| ATOM | 768 | OE1 | GLN | A | 97 | 14.107 | 27.041 | 21.771 | 1.00 | 52.10 | 8 | O |
| ATOM | 769 | NE2 | GLN | A | 97 | 12.432 | 27.980 | 20.476 | 1.00 | 49.48 | 7 | N |
| ATOM | 770 | N | ARG | A | 98 | 18.135 | 25.469 | 18.517 | 1.00 | 40.51 | 7 | N |
| ATOM | 771 | CA | ARG | A | 98 | 19.174 | 24.420 | 18.423 | 1.00 | 39.90 | 6 | C |
| ATOM | 772 | C | ARG | A | 98 | 20.281 | 24.661 | 19.415 | 1.00 | 39.65 | 6 | C |
| ATOM | 773 | O | ARG | A | 98 | 20.849 | 23.731 | 20.014 | 1.00 | 39.01 | 8 | O |
| ATOM | 774 | CB | ARG | A | 98 | 19.643 | 24.291 | 16.997 | 1.00 | 40.97 | 6 | C |
| ATOM | 775 | CG | ARG | A | 98 | 20.592 | 23.145 | 16.802 | 1.00 | 47.01 | 6 | C |
| ATOM | 776 | CD | ARG | A | 98 | 20.961 | 22.873 | 15.356 | 1.00 | 48.84 | 6 | C |
| ATOM | 777 | NE | ARG | A | 98 | 19.688 | 22.730 | 14.627 | 1.00 | 52.18 | 7 | N |
| ATOM | 778 | CZ | ARG | A | 98 | 19.404 | 23.711 | 13.696 | 1.00 | 54.11 | 6 | C |
| ATOM | 779 | NH1 | ARG | A | 98 | 20.263 | 24.734 | 13.485 | 1.00 | 54.13 | 7 | N |
| ATOM | 780 | NH2 | ARG | A | 98 | 18.264 | 23.565 | 13.056 | 1.00 | 51.43 | 7 | N |
| ATOM | 781 | N | PHE | A | 99 | 20.657 | 25.939 | 19.742 | 1.00 | 37.30 | 7 | N |
| ATOM | 782 | CA | PHE | A | 99 | 21.701 | 26.060 | 20.760 | 1.00 | 33.66 | 6 | C |
| ATOM | 783 | C | PHE | A | 99 | 21.164 | 26.169 | 22.186 | 1.00 | 34.15 | 6 | C |
| ATOM | 784 | O | PHE | A | 99 | 22.009 | 25.843 | 23.049 | 1.00 | 30.59 | 8 | O |
| ATOM | 785 | CB | PHE | A | 99 | 22.534 | 27.197 | 20.399 | 1.00 | 32.59 | 6 | C |
| ATOM | 786 | CG | PHE | A | 99 | 23.627 | 26.988 | 19.392 | 1.00 | 31.53 | 6 | C |
| ATOM | 787 | CD1 | PHE | A | 99 | 23.404 | 27.328 | 18.095 | 1.00 | 32.45 | 6 | C |
| ATOM | 788 | CD2 | PHE | A | 99 | 24.836 | 26.483 | 19.706 | 1.00 | 30.64 | 6 | C |
| ATOM | 789 | CE1 | PHE | A | 99 | 24.336 | 27.180 | 17.105 | 1.00 | 31.13 | 6 | C |
| ATOM | 790 | CE2 | PHE | A | 99 | 25.823 | 26.306 | 18.770 | 1.00 | 34.28 | 6 | C |
| ATOM | 791 | CZ | PHE | A | 99 | 25.559 | 26.659 | 17.457 | 1.00 | 31.49 | 6 | C |
| ATOM | 792 | N | TYR | A | 100 | 19.901 | 26.612 | 22.394 | 1.00 | 32.63 | 7 | N |
| ATOM | 793 | CA | TYR | A | 100 | 19.429 | 26.741 | 23.802 | 1.00 | 35.26 | 6 | C |
| ATOM | 794 | C | TYR | A | 100 | 18.130 | 25.979 | 23.946 | 1.00 | 38.30 | 6 | C |
| ATOM | 795 | O | TYR | A | 100 | 16.977 | 26.506 | 23.956 | 1.00 | 40.25 | 8 | O |
| ATOM | 796 | CB | TYR | A | 100 | 19.151 | 28.285 | 23.974 | 1.00 | 35.82 | 6 | C |
| ATOM | 797 | CG | TYR | A | 100 | 20.392 | 29.175 | 24.023 | 1.00 | 36.46 | 6 | C |
| ATOM | 798 | CD1 | TYR | A | 100 | 20.968 | 29.724 | 22.864 | 1.00 | 34.32 | 6 | C |
| ATOM | 799 | CD2 | TYR | A | 100 | 21.039 | 29.505 | 25.218 | 1.00 | 35.11 | 6 | C |
| ATOM | 800 | CE1 | TYR | A | 100 | 22.083 | 30.527 | 22.898 | 1.00 | 30.64 | 6 | C |
| ATOM | 801 | CE2 | TYR | A | 100 | 22.154 | 30.304 | 25.252 | 1.00 | 34.76 | 6 | C |
| ATOM | 802 | CZ | TYR | A | 100 | 22.675 | 30.814 | 24.072 | 1.00 | 33.66 | 6 | C |
| ATOM | 803 | OH | TYR | A | 100 | 23.820 | 31.635 | 24.106 | 1.00 | 36.77 | 8 | O |
| ATOM | 804 | N | PRO | A | 101 | 18.120 | 24.649 | 23.904 | 1.00 | 40.09 | 7 | N |
| ATOM | 805 | CA | PRO | A | 101 | 16.908 | 23.843 | 24.026 | 1.00 | 40.17 | 6 | C |
| ATOM | 806 | C | PRO | A | 101 | 16.228 | 24.097 | 25.386 | 1.00 | 40.13 | 6 | C |
| ATOM | 807 | O | PRO | A | 101 | 16.955 | 23.974 | 26.401 | 1.00 | 40.99 | 8 | O |
| ATOM | 808 | CB | PRO | A | 101 | 17.413 | 22.390 | 24.074 | 1.00 | 40.04 | 6 | C |
| ATOM | 809 | CG | PRO | A | 101 | 18.790 | 22.467 | 23.532 | 1.00 | 39.11 | 6 | C |
| ATOM | 810 | CD | PRO | A | 101 | 19.354 | 23.793 | 23.918 | 1.00 | 40.38 | 6 | C |
| ATOM | 811 | N | GLY | A | 102 | 14.949 | 24.382 | 25.354 | 1.00 | 40.76 | 7 | N |
| ATOM | 812 | CA | GLY | A | 102 | 14.222 | 24.718 | 26.601 | 1.00 | 44.59 | 6 | C |
| ATOM | 813 | C | GLY | A | 102 | 14.866 | 25.971 | 27.225 | 1.00 | 47.47 | 6 | C |
| ATOM | 814 | O | GLY | A | 102 | 14.799 | 26.143 | 28.462 | 1.00 | 49.92 | 8 | O |
| ATOM | 815 | N | LYS | A | 103 | 15.553 | 26.880 | 26.528 | 1.00 | 44.75 | 7 | N |
| ATOM | 816 | CA | LYS | A | 103 | 16.237 | 28.017 | 27.139 | 1.00 | 44.40 | 6 | C |
| ATOM | 817 | C | LYS | A | 103 | 16.254 | 29.267 | 26.270 | 1.00 | 43.17 | 6 | C |
| ATOM | 818 | O | LYS | A | 103 | 17.185 | 29.974 | 25.932 | 1.00 | 43.94 | 8 | O |
| ATOM | 819 | CB | LYS | A | 103 | 17.571 | 27.528 | 27.680 | 1.00 | 44.34 | 6 | C |
| ATOM | 820 | CG | LYS | A | 103 | 17.552 | 26.647 | 28.910 | 1.00 | 46.68 | 6 | C |
| ATOM | 821 | CD | LYS | A | 103 | 18.793 | 26.587 | 29.803 | 1.00 | 50.58 | 6 | C |
| ATOM | 822 | CE | LYS | A | 103 | 18.519 | 25.793 | 31.082 | 1.00 | 52.97 | 6 | C |
| ATOM | 823 | NZ | LYS | A | 103 | 17.647 | 26.586 | 32.060 | 1.00 | 55.23 | 7 | N |
| ATOM | 824 | N | VAL | A | 104 | 15.060 | 29.619 | 25.777 | 1.00 | 42.29 | 7 | N |
| ATOM | 825 | CA | VAL | A | 104 | 14.775 | 30.705 | 24.897 | 1.00 | 42.00 | 6 | C |
| ATOM | 826 | C | VAL | A | 104 | 13.456 | 31.343 | 25.398 | 1.00 | 43.92 | 6 | C |
| ATOM | 827 | O | VAL | A | 104 | 12.403 | 30.698 | 25.478 | 1.00 | 42.97 | 8 | O |
| ATOM | 828 | CB | VAL | A | 104 | 14.494 | 30.241 | 23.445 | 1.00 | 42.88 | 6 | C |
| ATOM | 829 | CG1 | VAL | A | 104 | 13.950 | 31.398 | 22.619 | 1.00 | 43.51 | 6 | C |
| ATOM | 830 | CG2 | VAL | A | 104 | 15.612 | 29.471 | 22.813 | 1.00 | 40.32 | 6 | C |
| ATOM | 831 | N | GLU | A | 105 | 13.621 | 32.610 | 25.692 | 1.00 | 43.72 | 7 | N |
| ATOM | 832 | CA | GLU | A | 105 | 12.761 | 33.620 | 26.190 | 1.00 | 41.83 | 6 | C |
| ATOM | 833 | C | GLU | A | 105 | 12.443 | 34.633 | 25.107 | 1.00 | 42.14 | 6 | C |
| ATOM | 834 | O | GLU | A | 105 | 13.210 | 34.806 | 24.153 | 1.00 | 45.11 | 8 | O |
| ATOM | 835 | CB | GLU | A | 105 | 13.624 | 34.303 | 27.291 | 1.00 | 38.98 | 6 | C |
| ATOM | 836 | CG | GLU | A | 105 | 13.983 | 33.371 | 28.439 | 1.00 | 41.72 | 6 | C |
| ATOM | 837 | CD | GLU | A | 105 | 12.928 | 33.052 | 29.517 | 1.00 | 42.10 | 6 | C |
| ATOM | 838 | OE1 | GLU | A | 105 | 11.789 | 33.532 | 29.315 | 1.00 | 40.77 | 8 | O |
| ATOM | 839 | OE2 | GLU | A | 105 | 13.236 | 32.343 | 30.526 | 1.00 | 40.18 | 8 | O |
| ATOM | 840 | N | ARG | A | 106 | 11.383 | 35.397 | 25.131 | 1.00 | 41.29 | 7 | N |
| ATOM | 841 | CA | ARG | A | 106 | 11.176 | 36.461 | 24.164 | 1.00 | 43.12 | 6 | C |
| ATOM | 842 | C | ARG | A | 106 | 10.605 | 37.720 | 24.849 | 1.00 | 45.10 | 6 | C |
| ATOM | 843 | O | ARG | A | 106 | 10.026 | 37.694 | 25.929 | 1.00 | 45.31 | 8 | O |
| ATOM | 844 | CB | ARG | A | 106 | 10.213 | 35.916 | 23.137 | 1.00 | 45.78 | 6 | C |
| ATOM | 845 | CG | ARG | A | 106 | 8.870 | 35.537 | 23.708 | 1.00 | 48.14 | 6 | C |
| ATOM | 846 | CD | ARG | A | 106 | 8.137 | 34.478 | 22.888 | 1.00 | 48.35 | 6 | C |
| ATOM | 847 | NE | ARG | A | 106 | 8.927 | 33.267 | 22.722 | 1.00 | 51.35 | 7 | N |
| ATOM | 848 | CZ | ARG | A | 106 | 9.603 | 32.813 | 21.691 | 1.00 | 52.60 | 6 | C |
| ATOM | 849 | NH1 | ARG | A | 106 | 9.621 | 33.520 | 20.569 | 1.00 | 55.59 | 7 | N |
| ATOM | 850 | NH2 | ARG | A | 106 | 10.285 | 31.679 | 21.679 | 1.00 | 52.87 | 7 | N |
| ATOM | 851 | N | ILE | A | 107 | 10.745 | 38.865 | 24.204 | 1.00 | 43.65 | 7 | N |
| ATOM | 852 | CA | ILE | A | 107 | 10.269 | 40.150 | 24.650 | 1.00 | 41.38 | 6 | C |
| ATOM | 853 | C | ILE | A | 107 | 8.854 | 40.191 | 24.048 | 1.00 | 43.58 | 6 | C |
| ATOM | 854 | O | ILE | A | 107 | 8.821 | 39.707 | 22.918 | 1.00 | 42.63 | 8 | O |
| ATOM | 855 | CB | ILE | A | 107 | 11.133 | 41.256 | 24.008 | 1.00 | 36.51 | 6 | C |
| ATOM | 856 | CG1 | ILE | A | 107 | 12.518 | 41.318 | 24.557 | 1.00 | 31.56 | 6 | C |
| ATOM | 857 | CG2 | ILE | A | 107 | 10.466 | 42.617 | 23.980 | 1.00 | 33.96 | 6 | C |
| ATOM | 858 | CD1 | ILE | A | 107 | 12.949 | 41.641 | 25.913 | 1.00 | 36.74 | 6 | C |
| ATOM | 859 | N | GLU | A | 108 | 7.835 | 40.800 | 24.700 | 1.00 | 44.37 | 7 | N |
| ATOM | 860 | CA | GLU | A | 108 | 6.540 | 40.792 | 24.025 | 1.00 | 43.69 | 6 | C |
| ATOM | 861 | C | GLU | A | 108 | 6.041 | 42.221 | 23.857 | 1.00 | 46.06 | 6 | C |
| ATOM | 862 | O | GLU | A | 108 | 6.484 | 43.206 | 24.491 | 1.00 | 47.24 | 8 | O |
| ATOM | 863 | CB | GLU | A | 108 | 5.504 | 40.045 | 24.919 | 0.00 | 25.00 | 6 | C |
| ATOM | 864 | CG | GLU | A | 108 | 6.016 | 38.639 | 25.119 | 0.00 | 25.00 | 6 | C |
| ATOM | 865 | CD | GLU | A | 108 | 5.329 | 37.654 | 26.028 | 0.00 | 25.00 | 6 | C |
| ATOM | 866 | OE1 | GLU | A | 108 | 4.431 | 38.059 | 26.836 | 0.00 | 25.00 | 8 | O |
| ATOM | 867 | OE2 | GLU | A | 108 | 5.773 | 36.451 | 25.855 | 0.00 | 25.00 | 8 | O |
| ATOM | 868 | N | ALA | A | 109 | 5.071 | 42.322 | 22.942 | 1.00 | 45.41 | 7 | N |
| ATOM | 869 | CA | ALA | A | 109 | 4.501 | 43.659 | 22.667 | 1.00 | 45.88 | 6 | C |
| ATOM | 870 | C | ALA | A | 109 | 3.910 | 44.084 | 24.007 | 1.00 | 45.31 | 6 | C |
| ATOM | 871 | O | ALA | A | 109 | 3.595 | 43.176 | 24.779 | 1.00 | 50.50 | 8 | O |
| ATOM | 872 | CB | ALA | A | 109 | 3.548 | 43.534 | 21.506 | 1.00 | 45.38 | 6 | C |
| ATOM | 873 | N | PRO | A | 110 | 3.800 | 45.374 | 24.302 | 1.00 | 42.28 | 7 | N |
| ATOM | 874 | CA | PRO | A | 110 | 4.237 | 46.413 | 23.349 | 1.00 | 40.12 | 6 | C |
| ATOM | 875 | C | PRO | A | 110 | 5.711 | 46.723 | 23.566 | 1.00 | 40.31 | 6 | C |
| ATOM | 876 | O | PRO | A | 110 | 6.115 | 47.802 | 23.125 | 1.00 | 39.46 | 8 | O |
| ATOM | 877 | CB | PRO | A | 110 | 3.314 | 47.563 | 23.768 | 1.00 | 38.26 | 6 | C |
| ATOM | 878 | CG | PRO | A | 110 | 3.321 | 47.474 | 25.280 | 1.00 | 40.82 | 6 | C |
| ATOM | 879 | CD | PRO | A | 110 | 3.318 | 45.976 | 25.562 | 1.00 | 40.91 | 6 | C |
| ATOM | 880 | N | GLY | A | 111 | 6.461 | 45.872 | 24.277 | 1.00 | 39.16 | 7 | N |
| ATOM | 881 | CA | GLY | A | 111 | 7.840 | 46.112 | 24.541 | 1.00 | 40.19 | 6 | C |
| ATOM | 882 | C | GLY | A | 111 | 8.643 | 46.053 | 23.243 | 1.00 | 41.90 | 6 | C |
| ATOM | 883 | O | GLY | A | 111 | 8.303 | 45.435 | 22.223 | 1.00 | 38.66 | 8 | O |
| ATOM | 884 | N | THR | A | 112 | 9.824 | 46.687 | 23.265 | 1.00 | 41.17 | 7 | N |
| ATOM | 885 | CA | THR | A | 112 | 10.676 | 46.697 | 22.069 | 1.00 | 40.27 | 6 | C |
| ATOM | 886 | C | THR | A | 112 | 12.117 | 46.654 | 22.537 | 1.00 | 40.12 | 6 | C |
| ATOM | 887 | O | THR | A | 112 | 12.398 | 47.313 | 23.506 | 1.00 | 39.55 | 8 | O |
| ATOM | 888 | CB | THR | A | 112 | 10.364 | 48.070 | 21.497 | 1.00 | 39.68 | 6 | C |
| ATOM | 889 | OG1 | THR | A | 112 | 9.735 | 48.090 | 20.247 | 1.00 | 42.26 | 8 | O |
| ATOM | 890 | CG2 | THR | A | 112 | 11.534 | 49.005 | 21.531 | 1.00 | 38.78 | 6 | C |
| ATOM | 891 | N | VAL | A | 113 | 13.037 | 45.933 | 21.922 | 1.00 | 38.93 | 7 | N |
| ATOM | 892 | CA | VAL | A | 113 | 14.430 | 45.783 | 22.191 | 1.00 | 36.67 | 6 | C |
| ATOM | 893 | C | VAL | A | 113 | 15.173 | 45.373 | 20.921 | 1.00 | 37.87 | 6 | C |
| ATOM | 894 | O | VAL | A | 113 | 14.792 | 44.603 | 20.019 | 1.00 | 37.62 | 8 | O |
| ATOM | 895 | CB | VAL | A | 113 | 14.761 | 44.622 | 23.181 | 1.00 | 36.65 | 6 | C |
| ATOM | 896 | CG1 | VAL | A | 113 | 16.258 | 44.564 | 23.459 | 1.00 | 30.81 | 6 | C |
| ATOM | 897 | CG2 | VAL | A | 113 | 13.901 | 44.606 | 24.450 | 1.00 | 33.03 | 6 | C |
| ATOM | 898 | N | GLU | A | 114 | 16.394 | 45.867 | 20.815 | 1.00 | 38.05 | 7 | N |
| ATOM | 899 | CA | GLU | A | 114 | 17.362 | 45.547 | 19.755 | 1.00 | 35.00 | 6 | C |
| ATOM | 900 | C | GLU | A | 114 | 18.674 | 45.366 | 20.509 | 1.00 | 32.56 | 6 | C |
| ATOM | 901 | O | GLU | A | 114 | 19.189 | 46.184 | 21.193 | 1.00 | 31.41 | 8 | O |
| ATOM | 902 | CB | GLU | A | 114 | 17.394 | 46.527 | 18.628 | 1.00 | 38.58 | 6 | C |
| ATOM | 903 | CG | GLU | A | 114 | 16.249 | 46.168 | 17.650 | 1.00 | 36.40 | 6 | C |
| ATOM | 904 | CD | GLU | A | 114 | 16.135 | 47.284 | 16.662 | 1.00 | 35.71 | 6 | C |
| ATOM | 905 | OE1 | GLU | A | 114 | 17.199 | 47.884 | 16.486 | 1.00 | 34.52 | 8 | O |
| ATOM | 906 | OE2 | GLU | A | 114 | 15.077 | 47.592 | 16.057 | 1.00 | 39.16 | 8 | O |
| ATOM | 907 | N | ALA | A | 115 | 19.194 | 44.155 | 20.376 | 1.00 | 30.00 | 7 | N |
| ATOM | 908 | CA | ALA | A | 115 | 20.335 | 43.696 | 21.138 | 1.00 | 29.49 | 6 | C |
| ATOM | 909 | C | ALA | A | 115 | 21.590 | 44.444 | 20.876 | 1.00 | 29.53 | 6 | C |
| ATOM | 910 | O | ALA | A | 115 | 22.563 | 44.299 | 21.602 | 1.00 | 31.95 | 8 | O |
| ATOM | 911 | CB | ALA | A | 115 | 20.133 | 42.179 | 21.191 | 1.00 | 29.07 | 6 | C |
| ATOM | 912 | N | GLY | A | 116 | 21.724 | 45.328 | 19.895 | 1.00 | 31.85 | 7 | N |
| ATOM | 913 | CA | GLY | A | 116 | 22.840 | 46.240 | 19.657 | 1.00 | 31.94 | 6 | C |
| ATOM | 914 | C | GLY | A | 116 | 22.744 | 47.370 | 20.662 | 1.00 | 34.52 | 6 | C |
| ATOM | 915 | O | GLY | A | 116 | 23.640 | 48.242 | 20.800 | 1.00 | 35.55 | 8 | O |
| ATOM | 916 | N | ASP | A | 117 | 21.641 | 47.378 | 21.455 | 1.00 | 34.55 | 7 | N |
| ATOM | 917 | CA | ASP | A | 117 | 21.574 | 48.358 | 22.542 | 1.00 | 36.04 | 6 | C |
| ATOM | 918 | C | ASP | A | 117 | 22.160 | 47.689 | 23.783 | 1.00 | 37.55 | 6 | C |
| ATOM | 919 | O | ASP | A | 117 | 22.252 | 48.404 | 24.724 | 1.00 | 38.40 | 8 | O |
| ATOM | 920 | CB | ASP | A | 117 | 20.148 | 48.750 | 22.861 | 1.00 | 33.87 | 6 | C |
| ATOM | 921 | CG | ASP | A | 117 | 19.850 | 49.909 | 21.899 | 1.00 | 37.67 | 6 | C |
| ATOM | 922 | OD1 | ASP | A | 117 | 20.873 | 50.677 | 21.707 | 1.00 | 40.16 | 8 | O |
| ATOM | 923 | OD2 | ASP | A | 117 | 18.704 | 49.989 | 21.419 | 1.00 | 32.57 | 8 | O |
| ATOM | 924 | N | ILE | A | 118 | 22.547 | 46.397 | 23.769 | 1.00 | 37.87 | 7 | N |
| ATOM | 925 | CA | ILE | A | 118 | 22.991 | 45.736 | 24.990 | 1.00 | 39.06 | 6 | C |
| ATOM | 926 | C | ILE | A | 118 | 24.469 | 45.474 | 25.098 | 1.00 | 40.33 | 6 | C |
| ATOM | 927 | O | ILE | A | 118 | 24.885 | 44.966 | 24.094 | 1.00 | 39.46 | 8 | O |
| ATOM | 928 | CB | ILE | A | 118 | 22.106 | 44.471 | 25.060 | 1.00 | 38.79 | 6 | C |
| ATOM | 929 | CG1 | ILE | A | 118 | 20.606 | 44.856 | 25.170 | 1.00 | 39.69 | 6 | C |
| ATOM | 930 | CG2 | ILE | A | 118 | 22.371 | 43.562 | 26.247 | 1.00 | 38.21 | 6 | C |
| ATOM | 931 | CD1 | ILE | A | 118 | 19.687 | 43.707 | 25.551 | 1.00 | 42.13 | 6 | C |
| ATOM | 932 | N | MET | A | 119 | 25.229 | 45.803 | 26.165 | 1.00 | 41.54 | 7 | N |
| ATOM | 933 | CA | MET | A | 119 | 26.662 | 45.475 | 26.237 | 1.00 | 38.31 | 6 | C |
| ATOM | 934 | C | MET | A | 119 | 26.817 | 44.542 | 27.470 | 1.00 | 38.93 | 6 | C |
| ATOM | 935 | O | MET | A | 119 | 26.437 | 44.882 | 28.624 | 1.00 | 41.46 | 8 | O |
| ATOM | 936 | CB | MET | A | 119 | 27.632 | 46.584 | 26.393 | 1.00 | 34.91 | 6 | C |
| ATOM | 937 | CG | MET | A | 119 | 29.034 | 46.328 | 26.875 | 1.00 | 34.29 | 6 | C |
| ATOM | 938 | SD | MET | A | 119 | 29.904 | 47.915 | 26.648 | 1.00 | 37.77 | 16 | S |
| ATOM | 939 | CE | MET | A | 119 | 29.000 | 48.848 | 28.022 | 1.00 | 36.89 | 6 | C |
| ATOM | 940 | N | MET | A | 120 | 27.389 | 43.405 | 27.146 | 1.00 | 36.43 | 7 | N |
| ATOM | 941 | CA | MET | A | 120 | 27.529 | 42.336 | 28.145 | 1.00 | 37.80 | 6 | C |
| ATOM | 942 | C | MET | A | 120 | 28.853 | 42.461 | 28.862 | 1.00 | 37.66 | 6 | C |
| ATOM | 943 | O | MET | A | 120 | 29.865 | 42.465 | 28.147 | 1.00 | 36.08 | 8 | O |
| ATOM | 944 | CB | MET | A | 120 | 27.212 | 40.965 | 27.498 | 1.00 | 33.68 | 6 | C |
| ATOM | 945 | CG | MET | A | 120 | 27.661 | 39.785 | 28.363 | 1.00 | 34.78 | 6 | C |
| ATOM | 946 | SD | MET | A | 120 | 27.577 | 38.142 | 27.521 | 1.00 | 30.80 | 16 | S |
| ATOM | 947 | CE | MET | A | 120 | 29.270 | 38.294 | 26.853 | 1.00 | 18.64 | 6 | C |
| ATOM | 948 | N | VAL | A | 121 | 28.769 | 42.649 | 30.201 | 1.00 | 37.29 | 7 | N |
| ATOM | 949 | CA | VAL | A | 121 | 30.047 | 42.767 | 30.938 | 1.00 | 38.31 | 6 | C |
| ATOM | 950 | C | VAL | A | 121 | 29.936 | 41.742 | 32.082 | 1.00 | 41.51 | 6 | C |
| ATOM | 951 | O | VAL | A | 121 | 29.038 | 41.742 | 32.950 | 1.00 | 43.73 | 8 | O |
| ATOM | 952 | CB | VAL | A | 121 | 30.437 | 44.109 | 31.471 | 1.00 | 39.13 | 6 | C |
| ATOM | 953 | CG1 | VAL | A | 121 | 31.740 | 44.207 | 32.275 | 1.00 | 36.25 | 6 | C |
| ATOM | 954 | CG2 | VAL | A | 121 | 30.531 | 45.101 | 30.335 | 1.00 | 38.99 | 6 | C |
| ATOM | 955 | N | GLY | A | 122 | 30.902 | 40.785 | 31.971 | 1.00 | 41.64 | 7 | N |
| ATOM | 956 | CA | GLY | A | 122 | 30.885 | 39.762 | 33.027 | 1.00 | 39.80 | 6 | C |
| ATOM | 957 | C | GLY | A | 122 | 29.457 | 39.258 | 32.949 | 1.00 | 42.33 | 6 | C |
| ATOM | 958 | O | GLY | A | 122 | 29.256 | 38.735 | 31.869 | 1.00 | 40.94 | 8 | O |
| ATOM | 959 | N | ASP | A | 123 | 28.615 | 39.375 | 33.984 | 1.00 | 45.79 | 7 | N |
| ATOM | 960 | CA | ASP | A | 123 | 27.271 | 38.767 | 33.884 | 1.00 | 44.61 | 6 | C |
| ATOM | 961 | C | ASP | A | 123 | 26.250 | 39.883 | 33.943 | 1.00 | 43.53 | 6 | C |
| ATOM | 962 | O | ASP | A | 123 | 25.073 | 39.551 | 34.179 | 1.00 | 42.53 | 8 | O |
| ATOM | 963 | CB | ASP | A | 123 | 26.912 | 37.764 | 34.839 | 0.00 | 25.00 | 6 | C |
| ATOM | 964 | CG | ASP | A | 123 | 27.276 | 36.395 | 34.264 | 0.00 | 25.00 | 6 | C |
| ATOM | 965 | OD1 | ASP | A | 123 | 27.654 | 36.220 | 33.073 | 0.00 | 25.00 | 8 | O |
| ATOM | 966 | OD2 | ASP | A | 123 | 27.189 | 35.398 | 35.018 | 0.00 | 25.00 | 8 | O |
| ATOM | 967 | N | HIS | A | 124 | 26.806 | 41.112 | 33.784 | 1.00 | 40.63 | 7 | N |
| ATOM | 968 | CA | HIS | A | 124 | 25.896 | 42.232 | 33.900 | 1.00 | 41.61 | 6 | C |
| ATOM | 969 | C | HIS | A | 124 | 25.639 | 42.713 | 32.484 | 1.00 | 43.02 | 6 | C |
| ATOM | 970 | O | HIS | A | 124 | 26.494 | 42.782 | 31.558 | 1.00 | 42.86 | 8 | O |
| ATOM | 971 | CB | HIS | A | 124 | 26.337 | 43.411 | 34.792 | 1.00 | 43.00 | 6 | C |
| ATOM | 972 | CG | HIS | A | 124 | 25.257 | 44.427 | 34.884 | 1.00 | 42.26 | 6 | C |
| ATOM | 973 | ND1 | HIS | A | 124 | 24.151 | 44.211 | 35.685 | 1.00 | 45.36 | 7 | N |
| ATOM | 974 | CD2 | HIS | A | 124 | 25.101 | 45.630 | 34.293 | 1.00 | 44.30 | 6 | C |
| ATOM | 975 | CE1 | HIS | A | 124 | 23.333 | 45.264 | 35.590 | 1.00 | 45.50 | 6 | C |
| ATOM | 976 | NE2 | HIS | A | 124 | 23.892 | 46.143 | 34.744 | 1.00 | 46.53 | 7 | N |
| ATOM | 977 | N | PHE | A | 125 | 24.326 | 43.045 | 32.288 | 1.00 | 42.09 | 7 | N |
| ATOM | 978 | CA | PHE | A | 125 | 23.991 | 43.530 | 30.952 | 1.00 | 41.35 | 6 | C |
| ATOM | 979 | C | PHE | A | 125 | 23.595 | 44.989 | 30.906 | 1.00 | 41.21 | 6 | C |
| ATOM | 980 | O | PHE | A | 125 | 22.483 | 45.316 | 31.348 | 1.00 | 43.44 | 8 | O |
| ATOM | 981 | CB | PHE | A | 125 | 22.895 | 42.642 | 30.284 | 1.00 | 41.55 | 6 | C |
| ATOM | 982 | CG | PHE | A | 125 | 23.410 | 41.264 | 29.919 | 1.00 | 39.91 | 6 | C |
| ATOM | 983 | CD1 | PHE | A | 125 | 23.752 | 40.364 | 30.906 | 1.00 | 36.89 | 6 | C |
| ATOM | 984 | CD2 | PHE | A | 125 | 23.533 | 40.907 | 28.566 | 1.00 | 39.75 | 6 | C |
| ATOM | 985 | CE1 | PHE | A | 125 | 24.223 | 39.115 | 30.561 | 1.00 | 40.59 | 6 | C |
| ATOM | 986 | CE2 | PHE | A | 125 | 24.001 | 39.654 | 28.220 | 1.00 | 40.95 | 6 | C |
| ATOM | 987 | CZ | PHE | A | 125 | 24.355 | 38.740 | 29.235 | 1.00 | 42.30 | 6 | C |
| ATOM | 988 | N | TYR | A | 126 | 24.401 | 45.887 | 30.348 | 1.00 | 38.97 | 7 | N |
| ATOM | 989 | CA | TYR | A | 126 | 23.992 | 47.298 | 30.272 | 1.00 | 39.21 | 6 | C |
| ATOM | 990 | C | TYR | A | 126 | 23.092 | 47.459 | 29.062 | 1.00 | 42.09 | 6 | C |
| ATOM | 991 | O | TYR | A | 126 | 23.473 | 47.058 | 27.947 | 1.00 | 45.16 | 8 | O |
| ATOM | 992 | CB | TYR | A | 126 | 25.146 | 48.235 | 30.154 | 1.00 | 40.40 | 6 | C |
| ATOM | 993 | CG | TYR | A | 126 | 26.133 | 48.224 | 31.276 | 1.00 | 42.20 | 6 | C |
| ATOM | 994 | CD1 | TYR | A | 126 | 27.220 | 47.392 | 31.288 | 1.00 | 40.99 | 6 | C |
| ATOM | 995 | CD2 | TYR | A | 126 | 25.919 | 49.122 | 32.368 | 1.00 | 45.58 | 6 | C |
| ATOM | 996 | CE1 | TYR | A | 126 | 28.112 | 47.429 | 32.374 | 1.00 | 45.13 | 6 | C |
| ATOM | 997 | CE2 | TYR | A | 126 | 26.811 | 49.151 | 33.443 | 1.00 | 45.27 | 6 | C |
| ATOM | 998 | CZ | TYR | A | 126 | 27.883 | 48.304 | 33.414 | 1.00 | 44.54 | 6 | C |
| ATOM | 999 | OH | TYR | A | 126 | 28.775 | 48.301 | 34.436 | 1.00 | 51.91 | 8 | O |
| ATOM | 1000 | N | ILE | A | 127 | 21.895 | 48.021 | 29.163 | 1.00 | 44.05 | 7 | N |
| ATOM | 1001 | CA | ILE | A | 127 | 20.901 | 48.158 | 28.112 | 1.00 | 41.92 | 6 | C |
| ATOM | 1002 | C | ILE | A | 127 | 20.493 | 49.601 | 27.859 | 1.00 | 43.99 | 6 | C |
| ATOM | 1003 | O | ILE | A | 127 | 19.976 | 50.339 | 28.734 | 1.00 | 49.40 | 8 | O |
| ATOM | 1004 | CB | ILE | A | 127 | 19.638 | 47.414 | 28.627 | 1.00 | 41.42 | 6 | C |
| ATOM | 1005 | CG1 | ILE | A | 127 | 19.809 | 45.978 | 29.111 | 1.00 | 41.15 | 6 | C |
| ATOM | 1006 | CG2 | ILE | A | 127 | 18.572 | 47.516 | 27.553 | 1.00 | 41.55 | 6 | C |
| ATOM | 1007 | CD1 | ILE | A | 127 | 18.539 | 45.207 | 29.523 | 1.00 | 36.62 | 6 | C |
| ATOM | 1008 | N | GLY | A | 128 | 20.670 | 50.173 | 26.692 | 1.00 | 42.61 | 7 | N |
| ATOM | 1009 | CA | GLY | A | 128 | 20.432 | 51.505 | 26.312 | 1.00 | 42.96 | 6 | C |
| ATOM | 1010 | C | GLY | A | 128 | 19.007 | 51.836 | 25.964 | 1.00 | 45.96 | 6 | C |
| ATOM | 1011 | O | GLY | A | 128 | 18.312 | 51.119 | 25.259 | 1.00 | 47.97 | 8 | O |
| ATOM | 1012 | N | GLU | A | 129 | 18.412 | 52.919 | 26.419 | 1.00 | 48.53 | 7 | N |
| ATOM | 1013 | CA | GLU | A | 129 | 17.015 | 53.193 | 26.060 | 1.00 | 48.14 | 6 | C |
| ATOM | 1014 | C | GLU | A | 129 | 17.194 | 53.954 | 24.763 | 1.00 | 45.84 | 6 | C |
| ATOM | 1015 | O | GLU | A | 129 | 18.060 | 54.808 | 24.716 | 1.00 | 43.60 | 8 | O |
| ATOM | 1016 | CB | GLU | A | 129 | 16.382 | 53.983 | 27.208 | 1.00 | 52.45 | 6 | C |
| ATOM | 1017 | CG | GLU | A | 129 | 14.854 | 54.026 | 27.025 | 1.00 | 55.35 | 6 | C |
| ATOM | 1018 | CD | GLU | A | 129 | 14.163 | 54.721 | 28.201 | 1.00 | 57.65 | 6 | C |
| ATOM | 1019 | OE1 | GLU | A | 129 | 14.794 | 54.965 | 29.270 | 1.00 | 58.86 | 8 | O |
| ATOM | 1020 | OE2 | GLU | A | 129 | 12.957 | 55.006 | 27.988 | 1.00 | 56.11 | 8 | O |
| ATOM | 1021 | N | SER | A | 130 | 16.492 | 53.734 | 23.671 | 1.00 | 42.83 | 7 | N |
| ATOM | 1022 | CA | SER | A | 130 | 16.815 | 54.487 | 22.463 | 1.00 | 43.25 | 6 | C |
| ATOM | 1023 | C | SER | A | 130 | 15.652 | 54.490 | 21.508 | 1.00 | 42.94 | 6 | C |
| ATOM | 1024 | O | SER | A | 130 | 14.628 | 53.860 | 21.802 | 1.00 | 42.70 | 8 | O |
| ATOM | 1025 | CB | SER | A | 130 | 17.985 | 53.797 | 21.715 | 1.00 | 45.67 | 6 | C |
| ATOM | 1026 | OG | SER | A | 130 | 17.563 | 52.538 | 21.135 | 1.00 | 47.55 | 8 | O |
| ATOM | 1027 | N | ALA | A | 131 | 15.852 | 55.037 | 20.287 | 1.00 | 44.08 | 7 | N |
| ATOM | 1028 | CA | ALA | A | 131 | 14.641 | 54.914 | 19.419 | 1.00 | 47.62 | 6 | C |
| ATOM | 1029 | C | ALA | A | 131 | 14.180 | 53.463 | 19.288 | 1.00 | 47.16 | 6 | C |
| ATOM | 1030 | O | ALA | A | 131 | 13.027 | 53.086 | 19.245 | 1.00 | 50.97 | 8 | O |
| ATOM | 1031 | CB | ALA | A | 131 | 14.861 | 55.440 | 18.032 | 1.00 | 47.05 | 6 | C |
| ATOM | 1032 | N | ARG | A | 132 | 15.144 | 52.564 | 19.235 | 1.00 | 45.36 | 7 | N |
| ATOM | 1033 | CA | ARG | A | 132 | 14.941 | 51.163 | 19.035 | 1.00 | 43.62 | 6 | C |
| ATOM | 1034 | C | ARG | A | 132 | 14.637 | 50.375 | 20.285 | 1.00 | 41.28 | 6 | C |
| ATOM | 1035 | O | ARG | A | 132 | 13.956 | 49.405 | 20.031 | 1.00 | 40.20 | 8 | O |
| ATOM | 1036 | CB | ARG | A | 132 | 16.217 | 50.764 | 18.225 | 1.00 | 44.19 | 6 | C |
| ATOM | 1037 | CG | ARG | A | 132 | 16.403 | 51.228 | 16.889 | 0.00 | 25.00 | 6 | C |
| ATOM | 1038 | CD | ARG | A | 132 | 15.327 | 50.920 | 15.863 | 0.00 | 25.00 | 6 | C |
| ATOM | 1039 | NE | ARG | A | 132 | 15.808 | 51.476 | 14.589 | 0.00 | 25.00 | 7 | N |
| ATOM | 1040 | CZ | ARG | A | 132 | 16.711 | 50.914 | 13.749 | 0.00 | 25.00 | 6 | C |
| ATOM | 1041 | NH1 | ARG | A | 132 | 17.287 | 49.728 | 14.010 | 0.00 | 25.00 | 7 | N |
| ATOM | 1042 | NH2 | ARG | A | 132 | 17.017 | 51.591 | 12.621 | 0.00 | 25.00 | 7 | N |
| ATOM | 1043 | N | THR | A | 133 | 15.078 | 50.659 | 21.473 | 1.00 | 40.43 | 7 | N |
| ATOM | 1044 | CA | THR | A | 133 | 14.706 | 49.880 | 22.646 | 1.00 | 42.15 | 6 | C |
| ATOM | 1045 | C | THR | A | 133 | 13.948 | 50.717 | 23.711 | 1.00 | 42.45 | 6 | C |
| ATOM | 1046 | O | THR | A | 133 | 14.574 | 51.483 | 24.448 | 1.00 | 42.32 | 8 | O |
| ATOM | 1047 | CB | THR | A | 133 | 15.898 | 49.286 | 23.401 | 1.00 | 42.19 | 6 | C |
| ATOM | 1048 | OG1 | THR | A | 133 | 16.463 | 48.440 | 22.399 | 1.00 | 40.39 | 8 | O |
| ATOM | 1049 | CG2 | THR | A | 133 | 15.370 | 48.522 | 24.592 | 1.00 | 40.77 | 6 | C |
| ATOM | 1050 | N | ASN | A | 134 | 12.628 | 50.508 | 23.713 | 1.00 | 41.43 | 7 | N |
| ATOM | 1051 | CA | ASN | A | 134 | 11.715 | 51.233 | 24.617 | 1.00 | 39.27 | 6 | C |
| ATOM | 1052 | C | ASN | A | 134 | 11.823 | 50.839 | 26.059 | 1.00 | 39.24 | 6 | C |
| ATOM | 1053 | O | ASN | A | 134 | 12.553 | 49.882 | 26.370 | 1.00 | 42.19 | 8 | O |
| ATOM | 1054 | CB | ASN | A | 134 | 10.287 | 51.135 | 24.022 | 1.00 | 35.65 | 6 | C |
| ATOM | 1055 | CG | ASN | A | 134 | 9.629 | 49.803 | 24.220 | 1.00 | 35.95 | 6 | C |
| ATOM | 1056 | OD1 | ASN | A | 134 | 10.017 | 48.909 | 25.008 | 1.00 | 36.84 | 8 | O |
| ATOM | 1057 | ND2 | ASN | A | 134 | 8.550 | 49.649 | 23.451 | 1.00 | 33.02 | 7 | N |
| ATOM | 1058 | N | ALA | A | 135 | 11.166 | 51.508 | 26.988 | 1.00 | 39.69 | 7 | N |
| ATOM | 1059 | CA | ALA | A | 135 | 11.254 | 51.235 | 28.409 | 1.00 | 41.26 | 6 | C |
| ATOM | 1060 | C | ALA | A | 135 | 10.756 | 49.868 | 28.824 | 1.00 | 40.43 | 6 | C |
| ATOM | 1061 | O | ALA | A | 135 | 11.320 | 49.135 | 29.627 | 1.00 | 40.79 | 8 | O |
| ATOM | 1062 | CB | ALA | A | 135 | 10.390 | 52.216 | 29.213 | 1.00 | 39.72 | 6 | C |
| ATOM | 1063 | N | GLU | A | 136 | 9.609 | 49.565 | 28.202 | 1.00 | 41.51 | 7 | N |
| ATOM | 1064 | CA | GLU | A | 136 | 8.921 | 48.307 | 28.507 | 1.00 | 41.00 | 6 | C |
| ATOM | 1065 | C | GLU | A | 136 | 9.807 | 47.137 | 28.151 | 1.00 | 43.34 | 6 | C |
| ATOM | 1066 | O | GLU | A | 136 | 9.947 | 46.116 | 28.855 | 1.00 | 44.33 | 8 | O |
| ATOM | 1067 | CB | GLU | A | 136 | 7.573 | 48.280 | 27.820 | 1.00 | 43.19 | 6 | C |
| ATOM | 1068 | CG | GLU | A | 136 | 6.699 | 47.092 | 28.214 | 1.00 | 47.13 | 6 | C |
| ATOM | 1069 | CD | GLU | A | 136 | 6.831 | 46.530 | 29.595 | 1.00 | 48.67 | 6 | C |
| ATOM | 1070 | OE1 | GLU | A | 136 | 7.237 | 47.177 | 30.616 | 1.00 | 52.69 | 8 | O |
| ATOM | 1071 | OE2 | GLU | A | 136 | 6.493 | 45.316 | 29.652 | 1.00 | 50.52 | 8 | O |
| ATOM | 1072 | N | GLY | A | 137 | 10.463 | 47.351 | 27.001 | 1.00 | 42.58 | 7 | N |
| ATOM | 1073 | CA | GLY | A | 137 | 11.427 | 46.384 | 26.515 | 1.00 | 41.79 | 6 | C |
| ATOM | 1074 | C | GLY | A | 137 | 12.591 | 46.256 | 27.462 | 1.00 | 41.10 | 6 | C |
| ATOM | 1075 | O | GLY | A | 137 | 12.886 | 45.077 | 27.805 | 1.00 | 43.59 | 8 | O |
| ATOM | 1076 | N | ALA | A | 138 | 13.241 | 47.325 | 27.888 | 1.00 | 38.62 | 7 | N |
| ATOM | 1077 | CA | ALA | A | 138 | 14.341 | 47.005 | 28.839 | 1.00 | 38.82 | 6 | C |
| ATOM | 1078 | C | ALA | A | 138 | 13.761 | 46.407 | 30.118 | 1.00 | 39.69 | 6 | C |
| ATOM | 1079 | O | ALA | A | 138 | 14.382 | 45.497 | 30.686 | 1.00 | 40.77 | 8 | O |
| ATOM | 1080 | CB | ALA | A | 138 | 15.224 | 48.179 | 29.090 | 1.00 | 39.67 | 6 | C |
| ATOM | 1081 | N | ARG | A | 139 | 12.584 | 46.854 | 30.530 | 1.00 | 40.13 | 7 | N |
| ATOM | 1082 | CA | ARG | A | 139 | 12.017 | 46.327 | 31.765 | 1.00 | 42.98 | 6 | C |
| ATOM | 1083 | C | ARG | A | 139 | 11.990 | 44.801 | 31.693 | 1.00 | 41.82 | 6 | C |
| ATOM | 1084 | O | ARG | A | 139 | 12.611 | 44.108 | 32.508 | 1.00 | 44.18 | 8 | O |
| ATOM | 1085 | CB | ARG | A | 139 | 10.607 | 46.917 | 32.036 | 1.00 | 46.03 | 6 | C |
| ATOM | 1086 | CG | ARG | A | 139 | 9.964 | 46.531 | 33.174 | 0.00 | 25.00 | 6 | C |
| ATOM | 1087 | CD | ARG | A | 139 | 8.476 | 46.846 | 33.125 | 0.00 | 25.00 | 6 | C |
| ATOM | 1088 | NE | ARG | A | 139 | 7.778 | 46.012 | 32.151 | 0.00 | 25.00 | 7 | N |
| ATOM | 1089 | CZ | ARG | A | 139 | 7.522 | 44.726 | 32.342 | 0.00 | 25.00 | 6 | C |
| ATOM | 1090 | NH1 | ARG | A | 139 | 7.904 | 44.146 | 33.471 | 0.00 | 25.00 | 7 | N |
| ATOM | 1091 | NH2 | ARG | A | 139 | 6.891 | 44.022 | 31.414 | 0.00 | 25.00 | 7 | N |
| ATOM | 1092 | N | GLN | A | 140 | 11.282 | 44.277 | 30.726 | 1.00 | 39.96 | 7 | N |
| ATOM | 1093 | CA | GLN | A | 140 | 11.117 | 42.869 | 30.448 | 1.00 | 39.67 | 6 | C |
| ATOM | 1094 | C | GLN | A | 140 | 12.462 | 42.180 | 30.341 | 1.00 | 41.42 | 6 | C |
| ATOM | 1095 | O | GLN | A | 140 | 12.716 | 41.103 | 30.948 | 1.00 | 45.50 | 8 | O |
| ATOM | 1096 | CB | GLN | A | 140 | 10.346 | 42.761 | 29.167 | 1.00 | 37.83 | 6 | C |
| ATOM | 1097 | CG | GLN | A | 140 | 8.827 | 42.602 | 29.143 | 1.00 | 36.27 | 6 | C |
| ATOM | 1098 | CD | GLN | A | 140 | 8.311 | 42.390 | 27.706 | 1.00 | 40.74 | 6 | C |
| ATOM | 1099 | OE1 | GLN | A | 140 | 8.679 | 41.352 | 27.056 | 1.00 | 42.86 | 8 | O |
| ATOM | 1100 | NE2 | GLN | A | 140 | 7.477 | 43.262 | 27.087 | 1.00 | 36.53 | 7 | N |
| ATOM | 1101 | N | MET | A | 141 | 13.390 | 42.817 | 29.615 | 1.00 | 41.08 | 7 | N |
| ATOM | 1102 | CA | MET | A | 141 | 14.715 | 42.234 | 29.468 | 1.00 | 39.01 | 6 | C |
| ATOM | 1103 | C | MET | A | 141 | 15.329 | 42.133 | 30.836 | 1.00 | 37.86 | 6 | C |
| ATOM | 1104 | O | MET | A | 141 | 15.689 | 40.979 | 31.086 | 1.00 | 39.52 | 8 | O |
| ATOM | 1105 | CB | MET | A | 141 | 15.665 | 42.985 | 28.523 | 1.00 | 41.84 | 6 | C |
| ATOM | 1106 | CG | MET | A | 141 | 17.049 | 42.300 | 28.317 | 1.00 | 41.33 | 6 | C |
| ATOM | 1107 | SD | MET | A | 141 | 16.917 | 40.673 | 27.510 | 1.00 | 43.02 | 16 | S |
| ATOM | 1108 | CE | MET | A | 141 | 16.580 | 40.996 | 25.803 | 1.00 | 39.58 | 6 | C |
| ATOM | 1109 | N | ILE | A | 142 | 15.439 | 43.186 | 31.669 | 1.00 | 36.75 | 7 | N |
| ATOM | 1110 | CA | ILE | A | 142 | 16.038 | 42.969 | 32.994 | 1.00 | 38.20 | 6 | C |
| ATOM | 1111 | C | ILE | A | 142 | 15.372 | 41.826 | 33.797 | 1.00 | 37.42 | 6 | C |
| ATOM | 1112 | O | ILE | A | 142 | 15.985 | 41.056 | 34.527 | 1.00 | 36.17 | 8 | O |
| ATOM | 1113 | CB | ILE | A | 142 | 15.911 | 44.334 | 33.709 | 1.00 | 40.47 | 6 | C |
| ATOM | 1114 | CG1 | ILE | A | 142 | 16.728 | 45.345 | 32.910 | 1.00 | 40.83 | 6 | C |
| ATOM | 1115 | CG2 | ILE | A | 142 | 16.293 | 44.230 | 35.163 | 1.00 | 41.86 | 6 | C |
| ATOM | 1116 | CD1 | ILE | A | 142 | 16.753 | 46.768 | 33.350 | 1.00 | 40.25 | 6 | C |
| ATOM | 1117 | N | ALA | A | 143 | 14.062 | 41.655 | 33.617 | 1.00 | 36.53 | 7 | N |
| ATOM | 1118 | CA | ALA | A | 143 | 13.292 | 40.648 | 34.301 | 1.00 | 39.03 | 6 | C |
| ATOM | 1119 | C | ALA | A | 143 | 13.765 | 39.255 | 33.886 | 1.00 | 44.10 | 6 | C |
| ATOM | 1120 | O | ALA | A | 143 | 13.974 | 38.366 | 34.749 | 1.00 | 44.96 | 8 | O |
| ATOM | 1121 | CB | ALA | A | 143 | 11.815 | 40.822 | 34.008 | 1.00 | 35.82 | 6 | C |
| ATOM | 1122 | N | ILE | A | 144 | 13.917 | 39.114 | 32.516 | 1.00 | 44.81 | 7 | N |
| ATOM | 1123 | CA | ILE | A | 144 | 14.407 | 37.802 | 32.055 | 1.00 | 42.08 | 6 | C |
| ATOM | 1124 | C | ILE | A | 144 | 15.812 | 37.547 | 32.537 | 1.00 | 43.57 | 6 | C |
| ATOM | 1125 | O | ILE | A | 144 | 15.977 | 36.393 | 32.984 | 1.00 | 46.89 | 8 | O |
| ATOM | 1126 | CB | ILE | A | 144 | 14.393 | 37.682 | 30.561 | 1.00 | 41.61 | 6 | C |
| ATOM | 1127 | CG1 | ILE | A | 144 | 12.920 | 37.640 | 30.112 | 1.00 | 40.08 | 6 | C |
| ATOM | 1128 | CG2 | ILE | A | 144 | 15.139 | 36.461 | 30.047 | 1.00 | 42.14 | 6 | C |
| ATOM | 1129 | CD1 | ILE | A | 144 | 12.611 | 38.327 | 28.800 | 1.00 | 37.77 | 6 | C |
| ATOM | 1130 | N | LEU | A | 145 | 16.695 | 38.534 | 32.520 | 1.00 | 42.59 | 7 | N |
| ATOM | 1131 | CA | LEU | A | 145 | 18.025 | 38.216 | 33.032 | 1.00 | 43.71 | 6 | C |
| ATOM | 1132 | C | LEU | A | 145 | 17.957 | 37.823 | 34.502 | 1.00 | 48.43 | 6 | C |
| ATOM | 1133 | O | LEU | A | 145 | 18.828 | 36.987 | 34.824 | 1.00 | 49.21 | 8 | O |
| ATOM | 1134 | CB | LEU | A | 145 | 18.927 | 39.433 | 32.790 | 1.00 | 39.36 | 6 | C |
| ATOM | 1135 | CG | LEU | A | 145 | 19.169 | 39.703 | 31.311 | 1.00 | 38.73 | 6 | C |
| ATOM | 1136 | CD1 | LEU | A | 145 | 19.773 | 41.064 | 31.029 | 1.00 | 39.04 | 6 | C |
| ATOM | 1137 | CD2 | LEU | A | 145 | 20.041 | 38.584 | 30.726 | 1.00 | 35.22 | 6 | C |
| ATOM | 1138 | N | GLU | A | 146 | 16.999 | 38.458 | 35.236 | 1.00 | 50.61 | 7 | N |
| ATOM | 1139 | CA | GLU | A | 146 | 16.856 | 38.201 | 36.662 | 1.00 | 53.29 | 6 | C |
| ATOM | 1140 | C | GLU | A | 146 | 16.438 | 36.762 | 36.933 | 1.00 | 50.86 | 6 | C |
| ATOM | 1141 | O | GLU | A | 146 | 17.061 | 36.035 | 37.724 | 1.00 | 49.36 | 8 | O |
| ATOM | 1142 | CB | GLU | A | 146 | 15.907 | 39.231 | 37.353 | 1.00 | 53.73 | 6 | C |
| ATOM | 1143 | CG | GLU | A | 146 | 16.777 | 40.233 | 38.120 | 1.00 | 57.30 | 6 | C |
| ATOM | 1144 | CD | GLU | A | 146 | 16.200 | 41.622 | 38.128 | 1.00 | 58.80 | 6 | C |
| ATOM | 1145 | OE1 | GLU | A | 146 | 14.964 | 41.606 | 37.938 | 1.00 | 61.75 | 8 | O |
| ATOM | 1146 | OE2 | GLU | A | 146 | 16.872 | 42.652 | 38.298 | 1.00 | 57.60 | 8 | O |
| ATOM | 1147 | N | LYS | A | 147 | 15.408 | 36.425 | 36.149 | 1.00 | 49.22 | 7 | N |
| ATOM | 1148 | CA | LYS | A | 147 | 14.886 | 35.051 | 36.300 | 1.00 | 50.37 | 6 | C |
| ATOM | 1149 | C | LYS | A | 147 | 15.993 | 34.027 | 36.128 | 1.00 | 53.27 | 6 | C |
| ATOM | 1150 | O | LYS | A | 147 | 16.044 | 32.960 | 36.738 | 1.00 | 56.64 | 8 | O |
| ATOM | 1151 | CB | LYS | A | 147 | 13.733 | 34.847 | 35.332 | 1.00 | 49.38 | 6 | C |
| ATOM | 1152 | CG | LYS | A | 147 | 13.286 | 33.440 | 35.003 | 1.00 | 48.97 | 6 | C |
| ATOM | 1153 | CD | LYS | A | 147 | 12.369 | 33.550 | 33.767 | 1.00 | 51.71 | 6 | C |
| ATOM | 1154 | CE | LYS | A | 147 | 12.017 | 32.131 | 33.251 | 1.00 | 54.44 | 6 | C |
| ATOM | 1155 | NZ | LYS | A | 147 | 11.005 | 32.155 | 32.118 | 1.00 | 57.75 | 7 | N |
| ATOM | 1156 | N | HIS | A | 148 | 16.999 | 34.240 | 35.278 | 1.00 | 52.72 | 7 | N |
| ATOM | 1157 | CA | HIS | A | 148 | 18.130 | 33.419 | 35.026 | 1.00 | 49.93 | 6 | C |
| ATOM | 1158 | C | HIS | A | 148 | 19.333 | 33.771 | 35.877 | 1.00 | 48.22 | 6 | C |
| ATOM | 1159 | O | HIS | A | 148 | 20.444 | 33.326 | 35.458 | 1.00 | 48.43 | 8 | O |
| ATOM | 1160 | CB | HIS | A | 148 | 18.532 | 33.502 | 33.500 | 1.00 | 50.21 | 6 | C |
| ATOM | 1161 | CG | HIS | A | 148 | 17.358 | 32.945 | 32.752 | 1.00 | 47.86 | 6 | C |
| ATOM | 1162 | ND1 | HIS | A | 148 | 17.163 | 31.576 | 32.798 | 1.00 | 48.79 | 7 | N |
| ATOM | 1163 | CD2 | HIS | A | 148 | 16.360 | 33.424 | 31.996 | 1.00 | 48.49 | 6 | C |
| ATOM | 1164 | CE1 | HIS | A | 148 | 16.061 | 31.303 | 32.067 | 1.00 | 48.15 | 6 | C |
| ATOM | 1165 | NE2 | HIS | A | 148 | 15.534 | 32.395 | 31.558 | 1.00 | 46.04 | 7 | N |
| ATOM | 1166 | N | GLY | A | 149 | 19.107 | 34.535 | 36.914 | 1.00 | 45.21 | 7 | N |
| ATOM | 1167 | CA | GLY | A | 149 | 20.302 | 34.812 | 37.736 | 1.00 | 46.95 | 6 | C |
| ATOM | 1168 | C | GLY | A | 149 | 21.329 | 35.711 | 37.147 | 1.00 | 48.16 | 6 | C |
| ATOM | 1169 | O | GLY | A | 149 | 22.481 | 35.631 | 37.653 | 1.00 | 51.80 | 8 | O |
| ATOM | 1170 | N | LEU | A | 150 | 21.020 | 36.531 | 36.141 | 1.00 | 47.23 | 7 | N |
| ATOM | 1171 | CA | LEU | A | 150 | 22.029 | 37.477 | 35.616 | 1.00 | 47.94 | 6 | C |
| ATOM | 1172 | C | LEU | A | 150 | 21.525 | 38.879 | 35.963 | 1.00 | 48.06 | 6 | C |
| ATOM | 1173 | O | LEU | A | 150 | 20.380 | 38.853 | 36.469 | 1.00 | 50.18 | 8 | O |
| ATOM | 1174 | CB | LEU | A | 150 | 22.192 | 37.298 | 34.118 | 1.00 | 48.24 | 6 | C |
| ATOM | 1175 | CG | LEU | A | 150 | 22.793 | 35.941 | 33.735 | 1.00 | 49.96 | 6 | C |
| ATOM | 1176 | CD1 | LEU | A | 150 | 22.539 | 35.635 | 32.269 | 1.00 | 50.41 | 6 | C |
| ATOM | 1177 | CD2 | LEU | A | 150 | 24.270 | 35.811 | 34.042 | 1.00 | 49.49 | 6 | C |
| ATOM | 1178 | N | SER | A | 151 | 22.137 | 39.996 | 35.754 | 1.00 | 47.20 | 7 | N |
| ATOM | 1179 | CA | SER | A | 151 | 21.581 | 41.304 | 36.146 | 1.00 | 47.89 | 6 | C |
| ATOM | 1180 | C | SER | A | 151 | 21.460 | 42.237 | 34.947 | 1.00 | 49.68 | 6 | C |
| ATOM | 1181 | O | SER | A | 151 | 22.050 | 42.012 | 33.854 | 1.00 | 53.62 | 8 | O |
| ATOM | 1182 | CB | SER | A | 151 | 22.633 | 41.954 | 37.084 | 1.00 | 47.40 | 6 | C |
| ATOM | 1183 | OG | SER | A | 151 | 23.870 | 41.747 | 36.321 | 1.00 | 52.12 | 8 | O |
| ATOM | 1184 | N | GLY | A | 152 | 20.825 | 43.380 | 35.011 | 1.00 | 49.88 | 7 | N |
| ATOM | 1185 | CA | GLY | A | 152 | 20.734 | 44.294 | 33.885 | 1.00 | 50.61 | 6 | C |
| ATOM | 1186 | C | GLY | A | 152 | 20.321 | 45.692 | 34.346 | 1.00 | 50.20 | 6 | C |
| ATOM | 1187 | O | GLY | A | 152 | 19.671 | 45.777 | 35.367 | 1.00 | 52.00 | 8 | O |
| ATOM | 1188 | N | SER | A | 153 | 20.813 | 46.703 | 33.658 | 1.00 | 50.84 | 7 | N |
| ATOM | 1189 | CA | SER | A | 153 | 20.532 | 48.089 | 34.035 | 1.00 | 49.59 | 6 | C |
| ATOM | 1190 | C | SER | A | 153 | 20.338 | 48.937 | 32.794 | 1.00 | 50.33 | 6 | C |
| ATOM | 1191 | O | SER | A | 153 | 20.696 | 48.553 | 31.665 | 1.00 | 52.58 | 8 | O |
| ATOM | 1192 | CB | SER | A | 153 | 21.643 | 48.644 | 34.902 | 1.00 | 48.22 | 6 | C |
| ATOM | 1193 | OG | SER | A | 153 | 22.845 | 48.444 | 34.240 | 1.00 | 47.32 | 8 | O |
| ATOM | 1194 | N | VAL | A | 154 | 19.691 | 50.069 | 32.919 | 1.00 | 47.74 | 7 | N |
| ATOM | 1195 | CA | VAL | A | 154 | 19.385 | 50.926 | 31.822 | 1.00 | 47.15 | 6 | C |
| ATOM | 1196 | C | VAL | A | 154 | 20.346 | 52.072 | 31.665 | 1.00 | 47.76 | 6 | C |
| ATOM | 1197 | O | VAL | A | 154 | 20.532 | 52.748 | 32.632 | 1.00 | 54.69 | 8 | O |
| ATOM | 1198 | CB | VAL | A | 154 | 17.967 | 51.461 | 31.935 | 1.00 | 47.44 | 6 | C |
| ATOM | 1199 | CG1 | VAL | A | 154 | 17.665 | 52.372 | 30.760 | 1.00 | 46.31 | 6 | C |
| ATOM | 1200 | CG2 | VAL | A | 154 | 17.062 | 50.246 | 32.028 | 1.00 | 45.82 | 6 | C |
| ATOM | 1201 | N | VAL | A | 155 | 20.969 | 52.239 | 30.525 | 1.00 | 46.98 | 7 | N |
| ATOM | 1202 | CA | VAL | A | 155 | 21.922 | 53.288 | 30.273 | 1.00 | 47.82 | 6 | C |
| ATOM | 1203 | C | VAL | A | 155 | 21.227 | 54.217 | 29.266 | 1.00 | 52.32 | 6 | C |
| ATOM | 1204 | O | VAL | A | 155 | 20.508 | 53.902 | 28.336 | 1.00 | 51.26 | 8 | O |
| ATOM | 1205 | CB | VAL | A | 155 | 23.292 | 52.892 | 29.714 | 1.00 | 45.92 | 6 | C |
| ATOM | 1206 | CG1 | VAL | A | 155 | 24.028 | 54.178 | 29.246 | 1.00 | 41.05 | 6 | C |
| ATOM | 1207 | CG2 | VAL | A | 155 | 24.049 | 52.066 | 30.746 | 1.00 | 44.34 | 6 | C |
| ATOM | 1208 | N | ARG | A | 156 | 21.384 | 55.481 | 29.512 | 1.00 | 56.85 | 7 | N |
| ATOM | 1209 | CA | ARG | A | 156 | 20.944 | 56.694 | 28.884 | 1.00 | 61.13 | 6 | C |
| ATOM | 1210 | C | ARG | A | 156 | 21.259 | 57.139 | 27.463 | 1.00 | 60.65 | 6 | C |
| ATOM | 1211 | O | ARG | A | 156 | 22.245 | 56.704 | 26.916 | 1.00 | 68.50 | 8 | O |
| ATOM | 1212 | CB | ARG | A | 156 | 21.622 | 57.746 | 29.831 | 1.00 | 63.95 | 6 | C |
| ATOM | 1213 | CG | ARG | A | 156 | 21.687 | 57.214 | 31.290 | 1.00 | 64.88 | 6 | C |
| ATOM | 1214 | CD | ARG | A | 156 | 22.915 | 57.687 | 32.009 | 0.00 | 25.00 | 6 | C |
| ATOM | 1215 | NE | ARG | A | 156 | 22.810 | 57.432 | 33.454 | 0.00 | 25.00 | 7 | N |
| ATOM | 1216 | CZ | ARG | A | 156 | 23.586 | 57.983 | 34.385 | 0.00 | 25.00 | 6 | C |
| ATOM | 1217 | NH1 | ARG | A | 156 | 24.553 | 58.818 | 34.034 | 0.00 | 25.00 | 7 | N |
| ATOM | 1218 | NH2 | ARG | A | 156 | 23.390 | 57.700 | 35.668 | 0.00 | 25.00 | 7 | N |
| ATOM | 1219 | N | LEU | A | 157 | 20.722 | 57.532 | 26.660 | 0.00 | 25.00 | 7 | N |
| ATOM | 1220 | CA | LEU | A | 157 | 21.081 | 57.650 | 25.255 | 0.00 | 25.00 | 6 | C |
| ATOM | 1221 | C | LEU | A | 157 | 20.428 | 58.798 | 24.523 | 0.00 | 25.00 | 6 | C |
| ATOM | 1222 | O | LEU | A | 157 | 19.216 | 58.985 | 24.607 | 0.00 | 25.00 | 8 | O |
| ATOM | 1223 | CB | LEU | A | 157 | 20.789 | 56.356 | 24.500 | 0.00 | 25.00 | 6 | C |
| ATOM | 1224 | CG | LEU | A | 157 | 22.011 | 55.579 | 24.005 | 0.00 | 25.00 | 6 | C |
| ATOM | 1225 | CD1 | LEU | A | 157 | 21.588 | 54.422 | 23.115 | 0.00 | 25.00 | 6 | C |
| ATOM | 1226 | CD2 | LEU | A | 157 | 22.976 | 56.497 | 23.267 | 0.00 | 25.00 | 6 | C |
| ATOM | 1227 | N | GLU | A | 158 | 21.220 | 59.559 | 23.756 | 0.00 | 25.00 | 7 | N |
| ATOM | 1228 | CA | GLU | A | 158 | 20.640 | 60.691 | 23.068 | 0.00 | 25.00 | 6 | C |
| ATOM | 1229 | C | GLU | A | 158 | 21.137 | 60.957 | 21.650 | 0.00 | 25.00 | 6 | C |
| ATOM | 1230 | O | GLU | A | 158 | 20.308 | 61.357 | 20.793 | 0.00 | 25.00 | 8 | O |
| ATOM | 1231 | CB | GLU | A | 158 | 20.771 | 61.968 | 23.922 | 0.00 | 25.00 | 6 | C |
| ATOM | 1232 | CG | GLU | A | 158 | 19.589 | 62.199 | 24.887 | 0.00 | 25.00 | 6 | C |
| ATOM | 1233 | CD | GLU | A | 158 | 19.602 | 63.581 | 25.530 | 0.00 | 25.00 | 6 | C |
| ATOM | 1234 | OE1 | GLU | A | 158 | 20.430 | 64.417 | 25.112 | 0.00 | 25.00 | 8 | O |
| ATOM | 1235 | OE2 | GLU | A | 158 | 18.790 | 63.832 | 26.450 | 0.00 | 25.00 | 8 | O |
| ATOM | 1236 | N | LYS | A | 159 | 22.462 | 60.772 | 21.433 | 0.00 | 25.00 | 7 | N |
| ATOM | 1237 | CA | LYS | A | 159 | 22.991 | 61.144 | 20.126 | 0.00 | 25.00 | 6 | C |
| ATOM | 1238 | C | LYS | A | 159 | 23.362 | 59.910 | 19.311 | 0.00 | 25.00 | 6 | C |
| ATOM | 1239 | O | LYS | A | 159 | 23.767 | 60.106 | 18.108 | 0.00 | 25.00 | 8 | O |
| ATOM | 1240 | CB | LYS | A | 159 | 24.208 | 62.058 | 20.280 | 0.00 | 25.00 | 6 | C |
| ATOM | 1241 | CG | LYS | A | 159 | 23.887 | 63.425 | 20.863 | 0.00 | 25.00 | 6 | C |
| ATOM | 1242 | CD | LYS | A | 159 | 25.139 | 64.279 | 20.988 | 0.00 | 25.00 | 6 | C |
| ATOM | 1243 | CE | LYS | A | 159 | 24.818 | 65.645 | 21.571 | 0.00 | 25.00 | 6 | C |
| ATOM | 1244 | NZ | LYS | A | 159 | 26.036 | 66.494 | 21.699 | 0.00 | 25.00 | 7 | N |
| ATOM | 1245 | N | VAL | A | 160 | 23.237 | 58.708 | 19.844 | 0.00 | 25.00 | 7 | N |
| ATOM | 1246 | CA | VAL | A | 160 | 23.567 | 57.564 | 18.991 | 0.00 | 25.00 | 6 | C |
| ATOM | 1247 | C | VAL | A | 160 | 22.378 | 56.607 | 18.980 | 0.00 | 25.00 | 6 | C |
| ATOM | 1248 | O | VAL | A | 160 | 21.535 | 56.495 | 19.896 | 0.00 | 25.00 | 8 | O |
| ATOM | 1249 | CB | VAL | A | 160 | 24.887 | 56.880 | 19.362 | 0.00 | 25.00 | 6 | C |
| ATOM | 1250 | CG1 | VAL | A | 160 | 25.955 | 57.388 | 18.365 | 0.00 | 25.00 | 6 | C |
| ATOM | 1251 | CG2 | VAL | A | 160 | 25.306 | 57.085 | 20.828 | 0.00 | 25.00 | 6 | C |
| ATOM | 1252 | N | LEU | A | 161 | 22.370 | 55.868 | 17.863 | 0.00 | 25.00 | 7 | N |
| ATOM | 1253 | CA | LEU | A | 161 | 21.290 | 54.930 | 17.580 | 0.00 | 25.00 | 6 | C |
| ATOM | 1254 | C | LEU | A | 161 | 21.295 | 53.769 | 18.569 | 0.00 | 25.00 | 6 | C |
| ATOM | 1255 | O | LEU | A | 161 | 20.224 | 53.278 | 19.002 | 0.00 | 25.00 | 8 | O |
| ATOM | 1256 | CB | LEU | A | 161 | 21.398 | 54.402 | 16.148 | 0.00 | 25.00 | 6 | C |
| ATOM | 1257 | CG | LEU | A | 161 | 21.211 | 55.431 | 15.031 | 0.00 | 25.00 | 6 | C |
| ATOM | 1258 | CD1 | LEU | A | 161 | 21.488 | 54.805 | 13.673 | 0.00 | 25.00 | 6 | C |
| ATOM | 1259 | CD2 | LEU | A | 161 | 19.787 | 55.964 | 15.026 | 0.00 | 25.00 | 6 | C |
| ATOM | 1260 | N | HIS | A | 162 | 22.353 | 53.238 | 18.779 | 1.00 | 54.50 | 7 | N |
| ATOM | 1261 | CA | HIS | A | 162 | 22.513 | 52.125 | 19.666 | 1.00 | 54.83 | 6 | C |
| ATOM | 1262 | C | HIS | A | 162 | 23.579 | 52.423 | 20.720 | 1.00 | 54.76 | 6 | C |
| ATOM | 1263 | O | HIS | A | 162 | 24.469 | 53.214 | 20.505 | 1.00 | 56.54 | 8 | O |
| ATOM | 1264 | CB | HIS | A | 162 | 22.964 | 50.820 | 19.007 | 1.00 | 58.46 | 6 | C |
| ATOM | 1265 | CG | HIS | A | 162 | 21.968 | 50.106 | 18.143 | 1.00 | 57.90 | 6 | C |
| ATOM | 1266 | ND1 | HIS | A | 162 | 22.302 | 49.386 | 17.034 | 1.00 | 57.48 | 7 | N |
| ATOM | 1267 | CD2 | HIS | A | 162 | 20.623 | 50.002 | 18.227 | 1.00 | 57.16 | 6 | C |
| ATOM | 1268 | CE1 | HIS | A | 162 | 21.196 | 48.880 | 16.489 | 1.00 | 57.03 | 6 | C |
| ATOM | 1269 | NE2 | HIS | A | 162 | 20.151 | 49.238 | 17.196 | 1.00 | 55.43 | 7 | N |
| ATOM | 1270 | N | LEU | A | 163 | 23.422 | 51.692 | 21.826 | 1.00 | 53.46 | 7 | N |
| ATOM | 1271 | CA | LEU | A | 163 | 24.376 | 51.832 | 22.898 | 1.00 | 49.16 | 6 | C |
| ATOM | 1272 | C | LEU | A | 163 | 25.753 | 51.557 | 22.314 | 1.00 | 46.30 | 6 | C |
| ATOM | 1273 | O | LEU | A | 163 | 26.580 | 52.390 | 22.573 | 1.00 | 44.20 | 8 | O |
| ATOM | 1274 | CB | LEU | A | 163 | 24.073 | 50.869 | 24.038 | 1.00 | 48.44 | 6 | C |
| ATOM | 1275 | CG | LEU | A | 163 | 25.226 | 50.956 | 25.082 | 1.00 | 50.03 | 6 | C |
| ATOM | 1276 | CD1 | LEU | A | 163 | 25.074 | 52.245 | 25.916 | 1.00 | 46.01 | 6 | C |
| ATOM | 1277 | CD2 | LEU | A | 163 | 25.163 | 49.607 | 25.721 | 1.00 | 48.34 | 6 | C |
| ATOM | 1278 | N | LYS | A | 164 | 25.871 | 50.461 | 21.546 | 1.00 | 46.62 | 7 | N |
| ATOM | 1279 | CA | LYS | A | 164 | 27.137 | 50.018 | 20.977 | 1.00 | 45.20 | 6 | C |
| ATOM | 1280 | C | LYS | A | 164 | 27.788 | 50.691 | 19.818 | 1.00 | 47.71 | 6 | C |
| ATOM | 1281 | O | LYS | A | 164 | 28.902 | 50.360 | 19.419 | 1.00 | 50.28 | 8 | O |
| ATOM | 1282 | CB | LYS | A | 164 | 27.088 | 48.498 | 20.720 | 1.00 | 38.46 | 6 | C |
| ATOM | 1283 | CG | LYS | A | 164 | 27.215 | 47.919 | 22.149 | 1.00 | 36.80 | 6 | C |
| ATOM | 1284 | CD | LYS | A | 164 | 27.653 | 46.479 | 22.220 | 1.00 | 33.88 | 6 | C |
| ATOM | 1285 | CE | LYS | A | 164 | 26.817 | 45.500 | 21.426 | 1.00 | 32.23 | 6 | C |
| ATOM | 1286 | NZ | LYS | A | 164 | 27.006 | 44.094 | 21.787 | 1.00 | 31.58 | 7 | N |
| ATOM | 1287 | N | THR | A | 165 | 27.192 | 51.714 | 19.236 | 1.00 | 49.65 | 7 | N |
| ATOM | 1288 | CA | THR | A | 165 | 27.765 | 52.538 | 18.197 | 1.00 | 48.05 | 6 | C |
| ATOM | 1289 | C | THR | A | 165 | 28.808 | 53.358 | 18.972 | 1.00 | 46.84 | 6 | C |
| ATOM | 1290 | O | THR | A | 165 | 29.770 | 53.816 | 18.377 | 1.00 | 47.71 | 8 | O |
| ATOM | 1291 | CB | THR | A | 165 | 26.764 | 53.577 | 17.660 | 1.00 | 50.31 | 6 | C |
| ATOM | 1292 | OG1 | THR | A | 165 | 25.530 | 52.992 | 17.213 | 1.00 | 52.28 | 8 | O |
| ATOM | 1293 | CG2 | THR | A | 165 | 27.337 | 54.375 | 16.492 | 1.00 | 48.02 | 6 | C |
| ATOM | 1294 | N | GLY | A | 166 | 28.663 | 53.538 | 20.290 | 1.00 | 45.73 | 7 | N |
| ATOM | 1295 | CA | GLY | A | 166 | 29.672 | 54.312 | 21.026 | 1.00 | 46.87 | 6 | C |
| ATOM | 1296 | C | GLY | A | 166 | 30.574 | 53.564 | 21.957 | 1.00 | 48.61 | 6 | C |
| ATOM | 1297 | O | GLY | A | 166 | 31.562 | 54.116 | 22.511 | 1.00 | 50.78 | 8 | O |
| ATOM | 1298 | N | LEU | A | 167 | 30.351 | 52.248 | 22.197 | 1.00 | 51.05 | 7 | N |
| ATOM | 1299 | CA | LEU | A | 167 | 31.282 | 51.570 | 23.141 | 1.00 | 49.56 | 6 | C |
| ATOM | 1300 | C | LEU | A | 167 | 31.554 | 50.124 | 22.804 | 1.00 | 47.50 | 6 | C |
| ATOM | 1301 | O | LEU | A | 167 | 30.836 | 49.457 | 22.079 | 1.00 | 46.08 | 8 | O |
| ATOM | 1302 | CB | LEU | A | 167 | 30.636 | 51.564 | 24.493 | 1.00 | 53.91 | 6 | C |
| ATOM | 1303 | CG | LEU | A | 167 | 29.953 | 52.557 | 25.396 | 1.00 | 55.64 | 6 | C |
| ATOM | 1304 | CD1 | LEU | A | 167 | 28.604 | 51.998 | 25.864 | 1.00 | 54.35 | 6 | C |
| ATOM | 1305 | CD2 | LEU | A | 167 | 30.824 | 52.831 | 26.621 | 1.00 | 56.89 | 6 | C |
| ATOM | 1306 | N | ALA | A | 168 | 32.536 | 49.501 | 23.435 | 1.00 | 47.59 | 7 | N |
| ATOM | 1307 | CA | ALA | A | 168 | 32.939 | 48.130 | 23.300 | 1.00 | 44.76 | 6 | C |
| ATOM | 1308 | C | ALA | A | 168 | 33.752 | 47.696 | 24.530 | 1.00 | 45.81 | 6 | C |
| ATOM | 1309 | O | ALA | A | 168 | 34.700 | 48.368 | 24.912 | 1.00 | 42.97 | 8 | O |
| ATOM | 1310 | CB | ALA | A | 168 | 33.861 | 47.948 | 22.113 | 1.00 | 47.12 | 6 | C |
| ATOM | 1311 | N | TYR | A | 169 | 33.272 | 46.564 | 25.078 | 1.00 | 45.72 | 7 | N |
| ATOM | 1312 | CA | TYR | A | 169 | 33.941 | 46.036 | 26.259 | 1.00 | 44.36 | 6 | C |
| ATOM | 1313 | C | TYR | A | 169 | 34.883 | 45.068 | 25.613 | 1.00 | 45.49 | 6 | C |
| ATOM | 1314 | O | TYR | A | 169 | 34.396 | 44.238 | 24.809 | 1.00 | 44.42 | 8 | O |
| ATOM | 1315 | CB | TYR | A | 169 | 32.986 | 45.296 | 27.165 | 1.00 | 46.80 | 6 | C |
| ATOM | 1316 | CG | TYR | A | 169 | 33.663 | 44.838 | 28.440 | 1.00 | 49.26 | 6 | C |
| ATOM | 1317 | CD1 | TYR | A | 169 | 34.386 | 45.721 | 29.257 | 1.00 | 46.45 | 6 | C |
| ATOM | 1318 | CD2 | TYR | A | 169 | 33.538 | 43.484 | 28.788 | 1.00 | 48.79 | 6 | C |
| ATOM | 1319 | CE1 | TYR | A | 169 | 34.969 | 45.209 | 30.399 | 1.00 | 46.50 | 6 | C |
| ATOM | 1320 | CE2 | TYR | A | 169 | 34.138 | 43.031 | 29.937 | 1.00 | 47.91 | 6 | C |
| ATOM | 1321 | CZ | TYR | A | 169 | 34.855 | 43.904 | 30.742 | 1.00 | 46.29 | 6 | C |
| ATOM | 1322 | OH | TYR | A | 169 | 35.403 | 43.336 | 31.853 | 1.00 | 49.09 | 8 | O |
| ATOM | 1323 | N | LEU | A | 170 | 36.176 | 45.221 | 25.895 | 1.00 | 44.47 | 7 | N |
| ATOM | 1324 | CA | LEU | A | 170 | 37.146 | 44.285 | 25.295 | 1.00 | 40.33 | 6 | C |
| ATOM | 1325 | C | LEU | A | 170 | 37.560 | 43.340 | 26.387 | 1.00 | 43.47 | 6 | C |
| ATOM | 1326 | O | LEU | A | 170 | 38.598 | 42.666 | 26.219 | 1.00 | 44.67 | 8 | O |
| ATOM | 1327 | CB | LEU | A | 170 | 38.328 | 44.992 | 24.665 | 1.00 | 36.40 | 6 | C |
| ATOM | 1328 | CG | LEU | A | 170 | 37.854 | 46.035 | 23.632 | 1.00 | 35.76 | 6 | C |
| ATOM | 1329 | CD1 | LEU | A | 170 | 38.993 | 46.919 | 23.111 | 1.00 | 29.27 | 6 | C |
| ATOM | 1330 | CD2 | LEU | A | 170 | 36.975 | 45.398 | 22.536 | 1.00 | 31.40 | 6 | C |
| ATOM | 1331 | N | GLU | A | 171 | 36.718 | 43.238 | 27.464 | 1.00 | 44.40 | 7 | N |
| ATOM | 1332 | CA | GLU | A | 171 | 37.077 | 42.315 | 28.543 | 1.00 | 45.41 | 6 | C |
| ATOM | 1333 | C | GLU | A | 171 | 38.249 | 42.901 | 29.307 | 1.00 | 47.70 | 6 | C |
| ATOM | 1334 | O | GLU | A | 171 | 38.954 | 43.817 | 28.876 | 1.00 | 46.80 | 8 | O |
| ATOM | 1335 | CB | GLU | A | 171 | 37.428 | 40.921 | 28.015 | 1.00 | 44.46 | 6 | C |
| ATOM | 1336 | CG | GLU | A | 171 | 36.245 | 40.245 | 27.368 | 1.00 | 42.80 | 6 | C |
| ATOM | 1337 | CD | GLU | A | 171 | 35.068 | 39.947 | 28.227 | 1.00 | 44.45 | 6 | C |
| ATOM | 1338 | OE1 | GLU | A | 171 | 35.133 | 39.476 | 29.402 | 1.00 | 46.66 | 8 | O |
| ATOM | 1339 | OE2 | GLU | A | 171 | 33.927 | 40.181 | 27.753 | 1.00 | 44.84 | 8 | O |
| ATOM | 1340 | N | HIS | A | 172 | 38.522 | 42.364 | 30.489 | 1.00 | 52.55 | 7 | N |
| ATOM | 1341 | CA | HIS | A | 172 | 39.634 | 42.838 | 31.334 | 1.00 | 55.97 | 6 | C |
| ATOM | 1342 | C | HIS | A | 172 | 39.420 | 44.260 | 31.785 | 1.00 | 57.54 | 6 | C |
| ATOM | 1343 | O | HIS | A | 172 | 40.352 | 45.085 | 31.868 | 1.00 | 60.45 | 8 | O |
| ATOM | 1344 | CB | HIS | A | 172 | 40.987 | 42.692 | 30.610 | 1.00 | 56.43 | 6 | C |
| ATOM | 1345 | CG | HIS | A | 172 | 41.196 | 41.309 | 30.076 | 1.00 | 56.53 | 6 | C |
| ATOM | 1346 | ND1 | HIS | A | 172 | 41.210 | 40.173 | 30.862 | 1.00 | 57.32 | 7 | N |
| ATOM | 1347 | CD2 | HIS | A | 172 | 41.400 | 40.876 | 28.820 | 1.00 | 57.05 | 6 | C |
| ATOM | 1348 | CE1 | HIS | A | 172 | 41.413 | 39.094 | 30.120 | 1.00 | 56.50 | 6 | C |
| ATOM | 1349 | NE2 | HIS | A | 172 | 41.531 | 39.500 | 28.879 | 1.00 | 56.76 | 7 | N |
| ATOM | 1350 | N | ASN | A | 173 | 38.174 | 44.660 | 32.030 | 1.00 | 58.11 | 7 | N |
| ATOM | 1351 | CA | ASN | A | 173 | 37.768 | 45.982 | 32.468 | 1.00 | 59.93 | 6 | C |
| ATOM | 1352 | C | ASN | A | 173 | 38.110 | 47.075 | 31.460 | 1.00 | 61.32 | 6 | C |
| ATOM | 1353 | O | ASN | A | 173 | 37.851 | 48.246 | 31.704 | 1.00 | 61.15 | 8 | O |
| ATOM | 1354 | CB | ASN | A | 173 | 38.286 | 46.342 | 33.869 | 1.00 | 60.29 | 6 | C |
| ATOM | 1355 | CG | ASN | A | 173 | 37.266 | 47.127 | 34.672 | 1.00 | 61.91 | 6 | C |
| ATOM | 1356 | OD1 | ASN | A | 173 | 36.784 | 48.190 | 34.274 | 1.00 | 61.34 | 8 | O |
| ATOM | 1357 | ND2 | ASN | A | 173 | 36.853 | 46.672 | 35.862 | 1.00 | 63.56 | 7 | N |
| ATOM | 1358 | N | ASN | A | 174 | 38.633 | 46.698 | 30.307 | 1.00 | 62.83 | 7 | N |
| ATOM | 1359 | CA | ASN | A | 174 | 39.016 | 47.556 | 29.202 | 1.00 | 64.03 | 6 | C |
| ATOM | 1360 | C | ASN | A | 174 | 37.723 | 47.928 | 28.506 | 1.00 | 63.34 | 6 | C |
| ATOM | 1361 | O | ASN | A | 174 | 36.888 | 47.037 | 28.377 | 1.00 | 62.55 | 8 | O |
| ATOM | 1362 | CB | ASN | A | 174 | 40.069 | 46.897 | 28.332 | 1.00 | 65.98 | 6 | C |
| ATOM | 1363 | CG | ASN | A | 174 | 41.289 | 46.616 | 29.210 | 1.00 | 67.54 | 6 | C |
| ATOM | 1364 | OD1 | ASN | A | 174 | 41.703 | 47.558 | 29.889 | 1.00 | 68.63 | 8 | O |
| ATOM | 1365 | ND2 | ASN | A | 174 | 41.774 | 45.384 | 29.154 | 1.00 | 67.94 | 7 | N |
| ATOM | 1366 | N | LEU | A | 175 | 37.520 | 49.211 | 28.172 | 1.00 | 62.18 | 7 | N |
| ATOM | 1367 | CA | LEU | A | 175 | 36.237 | 49.581 | 27.585 | 1.00 | 58.30 | 6 | C |
| ATOM | 1368 | C | LEU | A | 175 | 36.402 | 50.746 | 26.652 | 1.00 | 57.12 | 6 | C |
| ATOM | 1369 | O | LEU | A | 175 | 36.697 | 51.828 | 27.164 | 1.00 | 60.67 | 8 | O |
| ATOM | 1370 | CB | LEU | A | 175 | 35.272 | 49.989 | 28.707 | 1.00 | 58.46 | 6 | C |
| ATOM | 1371 | CG | LEU | A | 175 | 34.102 | 50.816 | 28.143 | 1.00 | 61.08 | 6 | C |
| ATOM | 1372 | CD1 | LEU | A | 175 | 32.892 | 49.932 | 27.898 | 1.00 | 60.97 | 6 | C |
| ATOM | 1373 | CD2 | LEU | A | 175 | 33.761 | 52.033 | 28.982 | 1.00 | 61.42 | 6 | C |
| ATOM | 1374 | N | LEU | A | 176 | 36.210 | 50.590 | 25.371 | 1.00 | 54.51 | 7 | N |
| ATOM | 1375 | CA | LEU | A | 176 | 36.356 | 51.633 | 24.377 | 1.00 | 53.17 | 6 | C |
| ATOM | 1376 | C | LEU | A | 176 | 35.152 | 52.537 | 24.511 | 1.00 | 53.04 | 6 | C |
| ATOM | 1377 | O | LEU | A | 176 | 34.048 | 52.001 | 24.660 | 1.00 | 53.75 | 8 | O |
| ATOM | 1378 | CB | LEU | A | 176 | 36.377 | 51.025 | 22.976 | 1.00 | 54.78 | 6 | C |
| ATOM | 1379 | CG | LEU | A | 176 | 37.507 | 50.109 | 22.516 | 1.00 | 54.22 | 6 | C |
| ATOM | 1380 | CD1 | LEU | A | 176 | 37.588 | 49.932 | 21.022 | 1.00 | 54.60 | 6 | C |
| ATOM | 1381 | CD2 | LEU | A | 176 | 38.852 | 50.679 | 22.982 | 1.00 | 55.24 | 6 | C |
| ATOM | 1382 | N | ALA | A | 177 | 35.283 | 53.855 | 24.508 | 1.00 | 53.05 | 7 | N |
| ATOM | 1383 | CA | ALA | A | 177 | 34.128 | 54.727 | 24.634 | 1.00 | 51.53 | 6 | C |
| ATOM | 1384 | C | ALA | A | 177 | 34.335 | 55.984 | 23.815 | 1.00 | 53.07 | 6 | C |
| ATOM | 1385 | O | ALA | A | 177 | 35.478 | 56.432 | 23.825 | 1.00 | 54.77 | 8 | O |
| ATOM | 1386 | CB | ALA | A | 177 | 33.904 | 55.111 | 26.072 | 1.00 | 50.70 | 6 | C |
| ATOM | 1387 | N | ALA | A | 178 | 33.342 | 56.493 | 23.152 | 1.00 | 53.99 | 7 | N |
| ATOM | 1388 | CA | ALA | A | 178 | 33.392 | 57.704 | 22.369 | 1.00 | 58.12 | 6 | C |
| ATOM | 1389 | C | ALA | A | 178 | 32.149 | 58.594 | 22.549 | 1.00 | 60.06 | 6 | C |
| ATOM | 1390 | O | ALA | A | 178 | 31.072 | 58.012 | 22.654 | 1.00 | 59.09 | 8 | O |
| ATOM | 1391 | CB | ALA | A | 178 | 33.414 | 57.357 | 20.871 | 1.00 | 57.76 | 6 | C |
| ATOM | 1392 | N | GLY | A | 179 | 32.260 | 59.929 | 22.544 | 1.00 | 62.70 | 7 | N |
| ATOM | 1393 | CA | GLY | A | 179 | 31.069 | 60.787 | 22.682 | 1.00 | 64.96 | 6 | C |
| ATOM | 1394 | C | GLY | A | 179 | 30.518 | 60.833 | 24.110 | 1.00 | 66.28 | 6 | C |
| ATOM | 1395 | O | GLY | A | 179 | 31.310 | 60.854 | 25.087 | 1.00 | 69.76 | 8 | O |
| ATOM | 1396 | N | GLU | A | 180 | 29.193 | 60.822 | 24.255 | 1.00 | 64.13 | 7 | N |
| ATOM | 1397 | CA | GLU | A | 180 | 28.615 | 60.791 | 25.604 | 1.00 | 64.06 | 6 | C |
| ATOM | 1398 | C | GLU | A | 180 | 29.344 | 59.867 | 26.563 | 1.00 | 62.92 | 6 | C |
| ATOM | 1399 | O | GLU | A | 180 | 30.059 | 60.268 | 27.498 | 1.00 | 67.41 | 8 | O |
| ATOM | 1400 | CB | GLU | A | 180 | 27.195 | 60.212 | 25.589 | 1.00 | 64.95 | 6 | C |
| ATOM | 1401 | CG | GLU | A | 180 | 26.564 | 60.315 | 24.220 | 1.00 | 67.71 | 6 | C |
| ATOM | 1402 | CD | GLU | A | 180 | 25.082 | 60.008 | 24.204 | 1.00 | 68.99 | 6 | C |
| ATOM | 1403 | OE1 | GLU | A | 180 | 24.441 | 59.866 | 25.258 | 1.00 | 69.92 | 8 | O |
| ATOM | 1404 | OE2 | GLU | A | 180 | 24.521 | 59.907 | 23.089 | 1.00 | 70.61 | 8 | O |
| ATOM | 1405 | N | PHE | A | 181 | 29.281 | 58.560 | 26.388 | 1.00 | 58.81 | 7 | N |
| ATOM | 1406 | CA | PHE | A | 181 | 29.869 | 57.540 | 27.213 | 1.00 | 55.50 | 6 | C |
| ATOM | 1407 | C | PHE | A | 181 | 31.222 | 57.759 | 27.831 | 1.00 | 57.61 | 6 | C |
| ATOM | 1408 | O | PHE | A | 181 | 31.576 | 57.020 | 28.792 | 1.00 | 54.58 | 8 | O |
| ATOM | 1409 | CB | PHE | A | 181 | 29.971 | 56.179 | 26.422 | 1.00 | 52.18 | 6 | C |
| ATOM | 1410 | CG | PHE | A | 181 | 28.647 | 56.017 | 25.713 | 1.00 | 49.11 | 6 | C |
| ATOM | 1411 | CD1 | PHE | A | 181 | 28.608 | 56.132 | 24.338 | 1.00 | 49.74 | 6 | C |
| ATOM | 1412 | CD2 | PHE | A | 181 | 27.477 | 55.785 | 26.411 | 1.00 | 46.45 | 6 | C |
| ATOM | 1413 | CE1 | PHE | A | 181 | 27.420 | 56.011 | 23.640 | 1.00 | 49.15 | 6 | C |
| ATOM | 1414 | CE2 | PHE | A | 181 | 26.296 | 55.665 | 25.757 | 1.00 | 47.30 | 6 | C |
| ATOM | 1415 | CZ | PHE | A | 181 | 26.254 | 55.775 | 24.369 | 1.00 | 50.04 | 6 | C |
| ATOM | 1416 | N | VAL | A | 182 | 32.031 | 58.679 | 27.277 | 1.00 | 60.15 | 7 | N |
| ATOM | 1417 | CA | VAL | A | 182 | 33.344 | 58.803 | 27.918 | 1.00 | 64.92 | 6 | C |
| ATOM | 1418 | C | VAL | A | 182 | 33.220 | 59.220 | 29.380 | 1.00 | 68.82 | 6 | C |
| ATOM | 1419 | O | VAL | A | 182 | 33.928 | 58.661 | 30.247 | 1.00 | 69.89 | 8 | O |
| ATOM | 1420 | CB | VAL | A | 182 | 34.151 | 59.825 | 27.129 | 1.00 | 66.58 | 6 | C |
| ATOM | 1421 | CG1 | VAL | A | 182 | 35.273 | 60.401 | 27.982 | 1.00 | 67.11 | 6 | C |
| ATOM | 1422 | CG2 | VAL | A | 182 | 34.637 | 59.167 | 25.837 | 1.00 | 67.47 | 6 | C |
| ATOM | 1423 | N | SER | A | 183 | 32.332 | 60.191 | 29.667 | 1.00 | 70.60 | 7 | N |
| ATOM | 1424 | CA | SER | A | 183 | 32.246 | 60.647 | 31.052 | 1.00 | 73.23 | 6 | C |
| ATOM | 1425 | C | SER | A | 183 | 31.083 | 60.107 | 31.827 | 1.00 | 74.75 | 6 | C |
| ATOM | 1426 | O | SER | A | 183 | 30.932 | 60.434 | 33.015 | 1.00 | 75.57 | 8 | O |
| ATOM | 1427 | CB | SER | A | 183 | 32.292 | 62.181 | 31.043 | 1.00 | 74.20 | 6 | C |
| ATOM | 1428 | OG | SER | A | 183 | 33.602 | 62.472 | 30.535 | 1.00 | 75.37 | 8 | O |
| ATOM | 1429 | N | LYS | A | 184 | 30.305 | 59.232 | 31.179 | 1.00 | 75.60 | 7 | N |
| ATOM | 1430 | CA | LYS | A | 184 | 29.176 | 58.630 | 31.925 | 1.00 | 74.23 | 6 | C |
| ATOM | 1431 | C | LYS | A | 184 | 29.766 | 58.056 | 33.201 | 1.00 | 73.12 | 6 | C |
| ATOM | 1432 | O | LYS | A | 184 | 30.791 | 57.401 | 33.170 | 1.00 | 73.55 | 8 | O |
| ATOM | 1433 | CB | LYS | A | 184 | 28.475 | 57.577 | 31.091 | 1.00 | 74.63 | 6 | C |
| ATOM | 1434 | CG | LYS | A | 184 | 27.593 | 58.028 | 30.077 | 0.00 | 25.00 | 6 | C |
| ATOM | 1435 | CD | LYS | A | 184 | 26.199 | 58.112 | 30.678 | 0.00 | 25.00 | 6 | C |
| ATOM | 1436 | CE | LYS | A | 184 | 25.132 | 57.826 | 29.633 | 0.00 | 25.00 | 6 | C |
| ATOM | 1437 | NZ | LYS | A | 184 | 25.203 | 58.779 | 28.491 | 0.00 | 25.00 | 7 | N |
| ATOM | 1438 | N | PRO | A | 185 | 29.128 | 58.333 | 34.312 | 1.00 | 73.30 | 7 | N |
| ATOM | 1439 | CA | PRO | A | 185 | 29.595 | 57.887 | 35.615 | 1.00 | 73.90 | 6 | C |
| ATOM | 1440 | C | PRO | A | 185 | 29.635 | 56.391 | 35.719 | 1.00 | 73.86 | 6 | C |
| ATOM | 1441 | O | PRO | A | 185 | 30.649 | 55.816 | 36.113 | 1.00 | 76.59 | 8 | O |
| ATOM | 1442 | CB | PRO | A | 185 | 28.605 | 58.492 | 36.609 | 1.00 | 74.32 | 6 | C |
| ATOM | 1443 | CG | PRO | A | 185 | 27.924 | 59.590 | 35.874 | 1.00 | 73.83 | 6 | C |
| ATOM | 1444 | CD | PRO | A | 185 | 27.890 | 59.136 | 34.425 | 1.00 | 73.36 | 6 | C |
| ATOM | 1445 | N | GLU | A | 186 | 28.564 | 55.719 | 35.332 | 1.00 | 73.22 | 7 | N |
| ATOM | 1446 | CA | GLU | A | 186 | 28.415 | 54.265 | 35.375 | 1.00 | 71.25 | 6 | C |
| ATOM | 1447 | C | GLU | A | 186 | 29.562 | 53.429 | 34.828 | 1.00 | 69.14 | 6 | C |
| ATOM | 1448 | O | GLU | A | 186 | 29.718 | 52.251 | 35.245 | 1.00 | 69.17 | 8 | O |
| ATOM | 1449 | CB | GLU | A | 186 | 27.098 | 53.872 | 34.683 | 1.00 | 70.86 | 6 | C |
| ATOM | 1450 | CG | GLU | A | 186 | 26.856 | 54.778 | 33.500 | 1.00 | 72.47 | 6 | C |
| ATOM | 1451 | CD | GLU | A | 186 | 25.447 | 55.295 | 33.340 | 1.00 | 73.12 | 6 | C |
| ATOM | 1452 | OE1 | GLU | A | 186 | 24.468 | 54.508 | 33.305 | 1.00 | 74.84 | 8 | O |
| ATOM | 1453 | OE2 | GLU | A | 186 | 25.305 | 56.533 | 33.245 | 1.00 | 73.47 | 8 | O |
| ATOM | 1454 | N | PHE | A | 187 | 30.370 | 54.005 | 33.932 | 1.00 | 65.17 | 7 | N |
| ATOM | 1455 | CA | PHE | A | 187 | 31.511 | 53.309 | 33.383 | 1.00 | 63.13 | 6 | C |
| ATOM | 1456 | C | PHE | A | 187 | 32.821 | 53.877 | 33.915 | 1.00 | 64.91 | 6 | C |
| ATOM | 1457 | O | PHE | A | 187 | 33.861 | 53.672 | 33.256 | 1.00 | 63.37 | 8 | O |
| ATOM | 1458 | CB | PHE | A | 187 | 31.558 | 53.405 | 31.825 | 1.00 | 59.18 | 6 | C |
| ATOM | 1459 | CG | PHE | A | 187 | 30.268 | 53.016 | 31.153 | 1.00 | 53.53 | 6 | C |
| ATOM | 1460 | CD1 | PHE | A | 187 | 29.745 | 53.775 | 30.131 | 1.00 | 51.23 | 6 | C |
| ATOM | 1461 | CD2 | PHE | A | 187 | 29.584 | 51.879 | 31.562 | 1.00 | 50.29 | 6 | C |
| ATOM | 1462 | CE1 | PHE | A | 187 | 28.555 | 53.367 | 29.556 | 1.00 | 48.73 | 6 | C |
| ATOM | 1463 | CE2 | PHE | A | 187 | 28.410 | 51.487 | 30.992 | 1.00 | 47.88 | 6 | C |
| ATOM | 1464 | CZ | PHE | A | 187 | 27.880 | 52.258 | 29.952 | 1.00 | 45.94 | 6 | C |
| ATOM | 1465 | N | GLN | A | 188 | 32.864 | 54.566 | 35.062 | 1.00 | 67.08 | 7 | N |
| ATOM | 1466 | CA | GLN | A | 188 | 34.199 | 55.113 | 35.422 | 1.00 | 68.70 | 6 | C |
| ATOM | 1467 | C | GLN | A | 188 | 35.138 | 54.220 | 36.161 | 1.00 | 69.43 | 6 | C |
| ATOM | 1468 | O | GLN | A | 188 | 36.372 | 54.443 | 36.103 | 1.00 | 72.41 | 8 | O |
| ATOM | 1469 | CB | GLN | A | 188 | 33.889 | 56.511 | 35.946 | 1.00 | 69.86 | 6 | C |
| ATOM | 1470 | CG | GLN | A | 188 | 33.063 | 57.343 | 34.973 | 1.00 | 70.31 | 6 | C |
| ATOM | 2471 | CD | GLN | A | 188 | 33.799 | 57.836 | 33.751 | 1.00 | 71.72 | 6 | C |
| ATOM | 1472 | OE1 | GLN | A | 188 | 34.950 | 58.281 | 33.887 | 1.00 | 71.14 | 8 | O |
| ATOM | 1473 | NE2 | GLN | A | 188 | 33.184 | 57.775 | 32.550 | 1.00 | 71.35 | 7 | N |
| ATOM | 1474 | N | ASP | A | 189 | 34.758 | 53.090 | 36.741 | 1.00 | 70.30 | 7 | N |
| ATOM | 1475 | CA | ASP | A | 189 | 35.723 | 52.176 | 37.362 | 1.00 | 70.94 | 6 | C |
| ATOM | 1476 | C | ASP | A | 189 | 36.305 | 51.258 | 36.278 | 1.00 | 71.04 | 6 | C |
| ATOM | 1477 | O | ASP | A | 189 | 36.893 | 50.217 | 36.583 | 1.00 | 71.25 | 8 | O |
| ATOM | 1478 | CB | ASP | A | 189 | 35.124 | 51.340 | 38.490 | 1.00 | 71.81 | 6 | C |
| ATOM | 1479 | CG | ASP | A | 189 | 34.775 | 52.260 | 39.641 | 0.00 | 25.00 | 6 | C |
| ATOM | 1480 | OD1 | ASP | A | 189 | 35.077 | 53.481 | 39.660 | 0.00 | 25.00 | 8 | O |
| ATOM | 1481 | OD2 | ASP | A | 189 | 34.123 | 51.669 | 40.538 | 0.00 | 25.00 | 8 | O |
| ATOM | 1482 | N | PHE | A | 190 | 36.106 | 51.608 | 35.003 | 1.00 | 70.18 | 7 | N |
| ATOM | 1483 | CA | PHE | A | 190 | 36.587 | 50.964 | 33.830 | 1.00 | 68.32 | 6 | C |
| ATOM | 1484 | C | PHE | A | 190 | 37.818 | 51.738 | 33.342 | 1.00 | 67.53 | 6 | C |
| ATOM | 1485 | O | PHE | A | 190 | 37.891 | 52.938 | 33.554 | 1.00 | 68.39 | 8 | O |
| ATOM | 1486 | CB | PHE | A | 190 | 35.718 | 51.001 | 32.543 | 1.00 | 65.29 | 6 | C |
| ATOM | 1487 | CG | PHE | A | 190 | 34.567 | 50.054 | 32.673 | 1.00 | 65.19 | 6 | C |
| ATOM | 1488 | CD1 | PHE | A | 190 | 33.323 | 50.482 | 33.082 | 1.00 | 64.27 | 6 | C |
| ATOM | 1489 | CD2 | PHE | A | 190 | 34.718 | 48.705 | 32.386 | 1.00 | 64.92 | 6 | C |
| ATOM | 1490 | CE1 | PHE | A | 190 | 32.251 | 49.630 | 33.210 | 1.00 | 63.20 | 6 | C |
| ATOM | 1491 | CE2 | PHE | A | 190 | 33.664 | 47.817 | 32.504 | 1.00 | 63.08 | 6 | C |
| ATOM | 1492 | CZ | PHE | A | 190 | 32.435 | 48.292 | 32.918 | 1.00 | 64.25 | 6 | C |
| ATOM | 1493 | N | ASN | A | 191 | 38.668 | 51.024 | 32.656 | 1.00 | 67.84 | 7 | N |
| ATOM | 1494 | CA | ASN | A | 191 | 39.833 | 51.619 | 32.033 | 1.00 | 69.14 | 6 | C |
| ATOM | 1495 | C | ASN | A | 191 | 39.275 | 52.227 | 30.736 | 1.00 | 70.15 | 6 | C |
| ATOM | 1496 | O | ASN | A | 191 | 39.398 | 51.726 | 29.620 | 1.00 | 71.20 | 8 | O |
| ATOM | 1497 | CB | ASN | A | 191 | 40.837 | 50.522 | 31.749 | 1.00 | 70.43 | 6 | C |
| ATOM | 1498 | CG | ASN | A | 191 | 42.133 | 51.041 | 31.155 | 1.00 | 74.02 | 6 | C |
| ATOM | 1499 | OD1 | ASN | A | 191 | 42.438 | 52.252 | 31.143 | 1.00 | 74.23 | 8 | O |
| ATOM | 1500 | ND2 | ASN | A | 191 | 42.922 | 50.076 | 30.646 | 1.00 | 75.60 | 7 | N |
| ATOM | 1501 | N | ILE | A | 192 | 38.569 | 53.351 | 30.801 | 1.00 | 70.10 | 7 | N |
| ATOM | 1502 | CA | ILE | A | 192 | 38.005 | 53.936 | 29.592 | 1.00 | 68.54 | 6 | C |
| ATOM | 1503 | C | ILE | A | 192 | 39.087 | 54.128 | 28.569 | 1.00 | 68.27 | 6 | C |
| ATOM | 1504 | O | ILE | A | 192 | 40.208 | 54.402 | 28.964 | 1.00 | 70.43 | 8 | O |
| ATOM | 1505 | CB | ILE | A | 192 | 37.240 | 55.199 | 29.982 | 1.00 | 68.93 | 6 | C |
| ATOM | 1506 | CG1 | ILE | A | 192 | 35.891 | 54.703 | 30.531 | 1.00 | 69.76 | 6 | C |
| ATOM | 1507 | CG2 | ILE | A | 192 | 37.089 | 56.092 | 28.782 | 1.00 | 68.75 | 6 | C |
| ATOM | 1508 | CD1 | ILE | A | 192 | 35.156 | 55.660 | 31.446 | 1.00 | 71.78 | 6 | C |
| ATOM | 1509 | N | ILE | A | 193 | 38.803 | 53.910 | 27.293 | 1.00 | 66.79 | 7 | N |
| ATOM | 1510 | CA | ILE | A | 193 | 39.711 | 54.066 | 26.184 | 1.00 | 65.12 | 6 | C |
| ATOM | 1511 | C | ILE | A | 193 | 38.923 | 54.852 | 25.149 | 1.00 | 68.03 | 6 | C |
| ATOM | 1512 | O | ILE | A | 193 | 37.992 | 54.379 | 24.495 | 1.00 | 69.41 | 8 | O |
| ATOM | 1513 | CB | ILE | A | 193 | 40.143 | 52.758 | 25.503 | 1.00 | 63.59 | 6 | C |
| ATOM | 1514 | CG1 | ILE | A | 193 | 40.971 | 51.924 | 26.478 | 1.00 | 65.29 | 6 | C |
| ATOM | 1515 | CG2 | ILE | A | 193 | 40.876 | 53.078 | 24.206 | 1.00 | 61.15 | 6 | C |
| ATOM | 1516 | CD1 | ILE | A | 193 | 42.001 | 51.014 | 25.806 | 1.00 | 67.25 | 6 | C |
| ATOM | 1517 | N | GLU | A | 194 | 39.123 | 56.044 | 24.733 | 1.00 | 68.29 | 7 | N |
| ATOM | 1518 | CA | GLU | A | 194 | 38.889 | 57.251 | 24.128 | 1.00 | 66.82 | 6 | C |
| ATOM | 1519 | C | GLU | A | 194 | 38.970 | 57.244 | 22.616 | 1.00 | 65.67 | 6 | C |
| ATOM | 1520 | O | GLU | A | 194 | 40.003 | 57.132 | 21.950 | 1.00 | 66.30 | 8 | O |
| ATOM | 1521 | CB | GLU | A | 194 | 39.892 | 58.291 | 24.637 | 1.00 | 68.66 | 6 | C |
| ATOM | 1522 | CG | GLU | A | 194 | 39.800 | 57.934 | 26.157 | 0.00 | 25.00 | 6 | C |
| ATOM | 1523 | CD | GLU | A | 194 | 40.848 | 58.903 | 26.668 | 0.00 | 25.00 | 6 | C |
| ATOM | 1524 | OE1 | GLU | A | 194 | 41.713 | 59.320 | 25.870 | 0.00 | 25.00 | 8 | O |
| ATOM | 1525 | OE2 | GLU | A | 194 | 40.802 | 59.247 | 27.869 | 0.00 | 25.00 | 8 | O |
| ATOM | 1526 | N | ILE | A | 195 | 37.702 | 57.319 | 22.136 | 1.00 | 61.00 | 7 | N |
| ATOM | 1527 | CA | ILE | A | 195 | 37.961 | 57.195 | 20.696 | 1.00 | 60.90 | 6 | C |
| ATOM | 1528 | C | ILE | A | 195 | 37.914 | 58.612 | 20.207 | 1.00 | 61.82 | 6 | C |
| ATOM | 1529 | O | ILE | A | 195 | 36.943 | 59.242 | 20.576 | 1.00 | 64.02 | 8 | O |
| ATOM | 1530 | CB | ILE | A | 195 | 37.155 | 56.016 | 20.185 | 1.00 | 58.85 | 6 | C |
| ATOM | 1531 | CG1 | ILE | A | 195 | 37.607 | 54.644 | 20.670 | 1.00 | 58.74 | 6 | C |
| ATOM | 1532 | CG2 | ILE | A | 195 | 37.224 | 56.122 | 18.680 | 1.00 | 54.89 | 6 | C |
| ATOM | 1533 | CD1 | ILE | A | 195 | 39.069 | 54.274 | 20.582 | 1.00 | 57.69 | 6 | C |
| ATOM | 1534 | N | PRO | A | 196 | 38.923 | 59.017 | 19.467 | 1.00 | 61.34 | 7 | N |
| ATOM | 1535 | CA | PRO | A | 196 | 38.980 | 60.333 | 18.885 | 1.00 | 63.28 | 6 | C |
| ATOM | 1536 | C | PRO | A | 196 | 37.769 | 60.521 | 17.977 | 1.00 | 65.53 | 6 | C |
| ATOM | 1537 | O | PRO | A | 196 | 37.455 | 59.613 | 17.211 | 1.00 | 66.74 | 8 | O |
| ATOM | 1538 | CB | PRO | A | 196 | 40.213 | 60.261 | 17.991 | 1.00 | 62.20 | 6 | C |
| ATOM | 1539 | CG | PRO | A | 196 | 41.062 | 59.159 | 18.542 | 1.00 | 62.55 | 6 | C |
| ATOM | 1540 | CD | PRO | A | 196 | 40.080 | 58.167 | 19.078 | 1.00 | 61.87 | 6 | C |
| ATOM | 1541 | N | GLU | A | 197 | 37.072 | 61.644 | 17.969 | 1.00 | 65.81 | 7 | N |
| ATOM | 1542 | CA | GLU | A | 197 | 35.896 | 61.787 | 17.120 | 1.00 | 65.73 | 6 | C |
| ATOM | 1543 | C | GLU | A | 197 | 36.151 | 61.579 | 15.646 | 1.00 | 64.55 | 6 | C |
| ATOM | 1544 | O | GLU | A | 197 | 35.227 | 61.107 | 14.943 | 1.00 | 61.75 | 8 | O |
| ATOM | 1545 | CB | GLU | A | 197 | 35.196 | 63.134 | 17.332 | 1.00 | 67.28 | 6 | C |
| ATOM | 1546 | CG | GLU | A | 197 | 34.012 | 63.405 | 16.579 | 0.00 | 25.00 | 6 | C |
| ATOM | 1547 | CD | GLU | A | 197 | 33.455 | 64.799 | 16.789 | 0.00 | 25.00 | 6 | C |
| ATOM | 1548 | OE1 | GLU | A | 197 | 34.083 | 65.585 | 17.529 | 0.00 | 25.00 | 8 | O |
| ATOM | 1549 | OE2 | GLU | A | 197 | 32.390 | 65.106 | 16.213 | 0.00 | 25.00 | 8 | O |
| ATOM | 1550 | N | GLU | A | 198 | 37.361 | 61.843 | 15.117 | 1.00 | 64.29 | 7 | N |
| ATOM | 1551 | CA | GLU | A | 198 | 37.547 | 61.575 | 13.666 | 1.00 | 63.21 | 6 | C |
| ATOM | 1552 | C | GLU | A | 198 | 37.470 | 60.048 | 13.500 | 1.00 | 62.44 | 6 | C |
| ATOM | 1553 | O | GLU | A | 198 | 37.082 | 59.632 | 12.414 | 1.00 | 64.04 | 8 | O |
| ATOM | 1554 | CB | GLU | A | 198 | 38.736 | 62.257 | 13.017 | 1.00 | 63.11 | 6 | C |
| ATOM | 1555 | CG | GLU | A | 198 | 38.854 | 63.560 | 12.947 | 0.00 | 25.00 | 6 | C |
| ATOM | 1556 | CD | GLU | A | 198 | 39.673 | 64.083 | 14.088 | 0.00 | 25.00 | 6 | C |
| ATOM | 1557 | OE1 | GLU | A | 198 | 40.184 | 63.247 | 14.864 | 0.00 | 25.00 | 8 | O |
| ATOM | 1558 | OE2 | GLU | A | 198 | 39.805 | 65.311 | 14.210 | 0.00 | 25.00 | 8 | O |
| ATOM | 1559 | N | GLU | A | 199 | 37.756 | 59.250 | 14.520 | 1.00 | 59.04 | 7 | N |
| ATOM | 1560 | CA | GLU | A | 199 | 37.674 | 57.812 | 14.540 | 1.00 | 57.74 | 6 | C |
| ATOM | 1561 | C | GLU | A | 199 | 36.488 | 57.305 | 15.363 | 1.00 | 55.26 | 6 | C |
| ATOM | 1562 | O | GLU | A | 199 | 36.479 | 56.229 | 15.959 | 1.00 | 55.68 | 8 | O |
| ATOM | 1563 | CB | GLU | A | 199 | 39.028 | 57.276 | 15.092 | 1.00 | 58.08 | 6 | C |
| ATOM | 1564 | CG | GLU | A | 199 | 40.197 | 57.343 | 14.110 | 1.00 | 61.18 | 6 | C |
| ATOM | 1565 | CD | GLU | A | 199 | 41.375 | 56.461 | 14.504 | 1.00 | 65.23 | 6 | C |
| ATOM | 1566 | OE1 | GLU | A | 199 | 41.460 | 55.614 | 15.464 | 1.00 | 66.14 | 8 | O |
| ATOM | 1567 | OE2 | GLU | A | 199 | 42.416 | 56.576 | 13.786 | 1.00 | 66.97 | 8 | O |
| ATOM | 1568 | N | SER | A | 200 | 35.380 | 57.990 | 15.534 | 1.00 | 54.20 | 7 | N |
| ATOM | 1569 | CA | SER | A | 200 | 34.264 | 57.468 | 16.356 | 1.00 | 55.68 | 6 | C |
| ATOM | 1570 | C | SER | A | 200 | 33.771 | 56.124 | 15.822 | 1.00 | 55.37 | 6 | C |
| ATOM | 1571 | O | SER | A | 200 | 33.535 | 55.163 | 16.607 | 1.00 | 57.10 | 8 | O |
| ATOM | 1572 | CB | SER | A | 200 | 33.190 | 58.510 | 16.625 | 1.00 | 57.62 | 6 | C |
| ATOM | 1573 | OG | SER | A | 200 | 32.422 | 58.898 | 15.481 | 1.00 | 59.81 | 8 | O |
| ATOM | 1574 | N | TYR | A | 201 | 33.658 | 55.870 | 14.514 | 1.00 | 51.39 | 7 | N |
| ATOM | 1575 | CA | TYR | A | 201 | 33.289 | 54.536 | 14.053 | 1.00 | 49.13 | 6 | C |
| ATOM | 1576 | C | TYR | A | 201 | 34.050 | 53.411 | 14.734 | 1.00 | 46.41 | 6 | C |
| ATOM | 1577 | O | TYR | A | 201 | 33.471 | 52.361 | 15.033 | 1.00 | 46.32 | 8 | O |
| ATOM | 1578 | CB | TYR | A | 201 | 33.376 | 54.384 | 12.536 | 1.00 | 48.20 | 6 | C |
| ATOM | 1579 | CG | TYR | A | 201 | 32.801 | 53.150 | 11.864 | 1.00 | 48.31 | 6 | C |
| ATOM | 1580 | CD1 | TYR | A | 201 | 31.446 | 52.833 | 11.909 | 1.00 | 46.03 | 6 | C |
| ATOM | 1581 | CD2 | TYR | A | 201 | 33.634 | 52.271 | 11.160 | 1.00 | 47.27 | 6 | C |
| ATOM | 1582 | CE1 | TYR | A | 201 | 30.930 | 51.717 | 11.299 | 1.00 | 44.02 | 6 | C |
| ATOM | 1583 | CE2 | TYR | A | 201 | 33.144 | 51.146 | 10.537 | 1.00 | 44.47 | 6 | C |
| ATOM | 1584 | CZ | TYR | A | 201 | 31.789 | 50.868 | 10.609 | 1.00 | 44.31 | 6 | C |
| ATOM | 1585 | OH | TYR | A | 201 | 31.315 | 49.730 | 9.978 | 1.00 | 39.36 | 8 | O |
| ATOM | 1586 | N | ALA | A | 202 | 35.328 | 53.475 | 15.027 | 1.00 | 47.02 | 7 | N |
| ATOM | 1587 | CA | ALA | A | 202 | 36.133 | 52.427 | 15.653 | 1.00 | 44.43 | 6 | C |
| ATOM | 1588 | C | ALA | A | 202 | 35.733 | 52.107 | 17.047 | 1.00 | 46.51 | 6 | C |
| ATOM | 1589 | O | ALA | A | 202 | 36.293 | 51.210 | 17.667 | 1.00 | 50.94 | 8 | O |
| ATOM | 1590 | CB | ALA | A | 202 | 37.602 | 52.780 | 15.673 | 1.00 | 42.28 | 6 | C |
| ATOM | 1591 | N | ALA | A | 203 | 34.760 | 52.749 | 17.670 | 1.00 | 47.90 | 7 | N |
| ATOM | 1592 | CA | ALA | A | 203 | 34.361 | 52.380 | 19.018 | 1.00 | 47.87 | 6 | C |
| ATOM | 1593 | C | ALA | A | 203 | 33.633 | 51.040 | 19.009 | 1.00 | 47.45 | 6 | C |
| ATOM | 1594 | O | ALA | A | 203 | 33.381 | 50.381 | 20.034 | 1.00 | 49.50 | 8 | O |
| ATOM | 1595 | CB | ALA | A | 203 | 33.475 | 53.542 | 19.470 | 1.00 | 49.39 | 6 | C |
| ATOM | 1596 | N | ASN | A | 204 | 33.235 | 50.554 | 17.852 | 1.00 | 45.04 | 7 | N |
| ATOM | 1597 | CA | ASN | A | 204 | 32.473 | 49.314 | 17.661 | 1.00 | 43.05 | 6 | C |
| ATOM | 1598 | C | ASN | A | 204 | 33.456 | 48.263 | 17.188 | 1.00 | 41.25 | 6 | C |
| ATOM | 1599 | O | ASN | A | 204 | 33.818 | 48.134 | 16.021 | 1.00 | 41.10 | 8 | O |
| ATOM | 1600 | CB | ASN | A | 204 | 31.261 | 49.579 | 16.784 | 1.00 | 41.59 | 6 | C |
| ATOM | 1601 | CG | ASN | A | 204 | 30.545 | 48.426 | 16.175 | 1.00 | 45.98 | 6 | C |
| ATOM | 1602 | OD1 | ASN | A | 204 | 30.765 | 47.242 | 16.492 | 1.00 | 49.13 | 8 | O |
| ATOM | 1603 | ND2 | ASN | A | 204 | 29.603 | 48.565 | 15.223 | 1.00 | 47.73 | 7 | N |
| ATOM | 1604 | N | CYS | A | 205 | 33.955 | 47.488 | 18.127 | 1.00 | 36.51 | 7 | N |
| ATOM | 1605 | CA | CYS | A | 205 | 34.986 | 46.521 | 17.788 | 1.00 | 33.94 | 6 | C |
| ATOM | 1606 | C | CYS | A | 205 | 34.516 | 45.291 | 18.543 | 1.00 | 35.73 | 6 | C |
| ATOM | 1607 | O | CYS | A | 205 | 33.765 | 45.586 | 19.453 | 1.00 | 34.23 | 8 | O |
| ATOM | 1608 | CB | CYS | A | 205 | 36.284 | 47.042 | 18.458 | 1.00 | 32.65 | 6 | C |
| ATOM | 1609 | SG | CYS | A | 205 | 37.001 | 48.237 | 17.296 | 1.00 | 40.44 | 16 | S |
| ATOM | 1610 | N | ILE | A | 206 | 35.002 | 44.096 | 18.233 | 1.00 | 34.95 | 7 | N |
| ATOM | 1611 | CA | ILE | A | 206 | 34.522 | 43.018 | 19.095 | 1.00 | 32.63 | 6 | C |
| ATOM | 1612 | C | ILE | A | 206 | 35.711 | 42.189 | 19.578 | 1.00 | 33.52 | 6 | C |
| ATOM | 1613 | O | ILE | A | 206 | 36.687 | 42.130 | 18.864 | 1.00 | 35.63 | 8 | O |
| ATOM | 1614 | CB | ILE | A | 206 | 33.633 | 42.078 | 18.349 | 1.00 | 31.19 | 6 | C |
| ATOM | 1615 | CG1 | ILE | A | 206 | 34.233 | 41.870 | 16.952 | 1.00 | 28.33 | 6 | C |
| ATOM | 1616 | CG2 | ILE | A | 206 | 32.175 | 42.449 | 18.289 | 1.00 | 32.94 | 6 | C |
| ATOM | 1617 | CD1 | ILE | A | 206 | 33.674 | 40.537 | 16.426 | 1.00 | 32.06 | 6 | C |
| ATOM | 1618 | N | TRP | A | 207 | 35.544 | 41.663 | 20.776 | 1.00 | 33.42 | 7 | N |
| ATOM | 1619 | CA | TRP | A | 207 | 36.530 | 40.895 | 21.417 | 1.00 | 34.40 | 6 | C |
| ATOM | 1620 | C | TRP | A | 207 | 36.245 | 39.445 | 21.077 | 1.00 | 36.42 | 6 | C |
| ATOM | 1621 | O | TRP | A | 207 | 35.087 | 39.046 | 21.243 | 1.00 | 35.39 | 8 | O |
| ATOM | 1622 | CB | TRP | A | 207 | 36.366 | 41.069 | 22.947 | 1.00 | 33.30 | 6 | C |
| ATOM | 1623 | CG | TRP | A | 207 | 37.287 | 40.108 | 23.700 | 1.00 | 30.66 | 6 | C |
| ATOM | 1624 | CD1 | TRP | A | 207 | 38.602 | 40.405 | 23.942 | 1.00 | 29.27 | 6 | C |
| ATOM | 1625 | CD2 | TRP | A | 207 | 36.989 | 38.838 | 24.240 | 1.00 | 27.00 | 6 | C |
| ATOM | 1626 | NE1 | TRP | A | 207 | 39.089 | 39.323 | 24.629 | 1.00 | 31.52 | 7 | N |
| ATOM | 1627 | CE2 | TRP | A | 207 | 38.171 | 38.360 | 24.835 | 1.00 | 26.28 | 6 | C |
| ATOM | 1628 | CE3 | TRP | A | 207 | 35.847 | 38.032 | 24.303 | 1.00 | 28.27 | 6 | C |
| ATOM | 1629 | CZ2 | TRP | A | 207 | 38.286 | 37.153 | 25.465 | 1.00 | 26.07 | 6 | C |
| ATOM | 1630 | CZ3 | TRP | A | 207 | 35.961 | 36.773 | 24.966 | 1.00 | 29.27 | 6 | C |
| ATOM | 1631 | CH2 | TRP | A | 207 | 37.180 | 36.344 | 25.536 | 1.00 | 28.64 | 6 | C |
| ATOM | 1632 | N | VAL | A | 208 | 37.213 | 38.628 | 20.651 | 1.00 | 36.26 | 7 | N |
| ATOM | 1633 | CA | VAL | A | 208 | 36.693 | 37.227 | 20.461 | 1.00 | 35.78 | 6 | C |
| ATOM | 1634 | C | VAL | A | 208 | 37.876 | 36.354 | 20.885 | 1.00 | 33.79 | 6 | C |
| ATOM | 1635 | O | VAL | A | 208 | 38.794 | 36.416 | 20.062 | 1.00 | 33.39 | 8 | O |
| ATOM | 1636 | CB | VAL | A | 208 | 36.261 | 36.772 | 19.046 | 1.00 | 35.18 | 6 | C |
| ATOM | 1637 | CG1 | VAL | A | 208 | 35.753 | 35.309 | 19.030 | 1.00 | 28.30 | 6 | C |
| ATOM | 1638 | CG2 | VAL | A | 208 | 35.147 | 37.646 | 18.498 | 1.00 | 35.67 | 6 | C |
| ATOM | 1639 | N | ASN | A | 209 | 37.776 | 35.707 | 22.019 | 1.00 | 34.60 | 7 | N |
| ATOM | 1640 | CA | ASN | A | 209 | 38.919 | 34.880 | 22.401 | 1.00 | 36.57 | 6 | C |
| ATOM | 1641 | C | ASN | A | 209 | 40.254 | 35.569 | 22.360 | 1.00 | 36.26 | 6 | C |
| ATOM | 1642 | O | ASN | A | 209 | 41.164 | 34.930 | 21.845 | 1.00 | 37.31 | 8 | O |
| ATOM | 1643 | CB | ASN | A | 209 | 38.862 | 33.593 | 21.556 | 1.00 | 35.62 | 6 | C |
| ATOM | 1644 | CG | ASN | A | 209 | 37.512 | 32.990 | 21.932 | 1.00 | 38.86 | 6 | C |
| ATOM | 1645 | OD1 | ASN | A | 209 | 36.828 | 33.243 | 22.935 | 1.00 | 40.55 | 8 | O |
| ATOM | 1646 | ND2 | ASN | A | 209 | 37.140 | 32.129 | 21.014 | 1.00 | 38.63 | 7 | N |
| ATOM | 1647 | N | GLU | A | 210 | 40.360 | 36.787 | 22.909 | 1.00 | 37.34 | 7 | N |
| ATOM | 1648 | CA | GLU | A | 210 | 41.628 | 37.466 | 22.986 | 1.00 | 40.53 | 6 | C |
| ATOM | 1649 | C | GLU | A | 210 | 42.060 | 38.094 | 21.718 | 1.00 | 41.59 | 6 | C |
| ATOM | 1650 | O | GLU | A | 210 | 43.191 | 38.592 | 21.728 | 1.00 | 47.48 | 8 | O |
| ATOM | 1651 | CB | GLU | A | 210 | 42.765 | 36.701 | 23.761 | 1.00 | 42.11 | 6 | C |
| ATOM | 1652 | CG | GLU | A | 210 | 42.229 | 36.413 | 25.257 | 0.00 | 25.00 | 6 | C |
| ATOM | 1653 | CD | GLU | A | 210 | 41.725 | 37.541 | 26.157 | 0.00 | 25.00 | 6 | C |
| ATOM | 1654 | OE1 | GLU | A | 210 | 41.660 | 38.750 | 25.822 | 0.00 | 25.00 | 8 | O |
| ATOM | 1655 | OE2 | GLU | A | 210 | 41.328 | 37.326 | 27.342 | 0.00 | 25.00 | 8 | O |
| ATOM | 1656 | N | ARG | A | 211 | 41.295 | 38.220 | 20.654 | 1.00 | 41.12 | 7 | N |
| ATOM | 1657 | CA | ARG | A | 211 | 41.667 | 38.999 | 19.484 | 1.00 | 40.95 | 6 | C |
| ATOM | 1658 | C | ARG | A | 211 | 40.539 | 40.023 | 19.348 | 1.00 | 42.26 | 6 | C |
| ATOM | 1659 | O | ARG | A | 211 | 39.418 | 39.901 | 19.903 | 1.00 | 44.73 | 8 | O |
| ATOM | 1660 | CB | ARG | A | 211 | 41.953 | 38.223 | 18.211 | 1.00 | 43.12 | 6 | C |
| ATOM | 1661 | CG | ARG | A | 211 | 41.398 | 36.796 | 18.458 | 1.00 | 43.12 | 6 | C |
| ATOM | 1662 | CD | ARG | A | 211 | 42.278 | 35.715 | 17.838 | 1.00 | 39.85 | 6 | C |
| ATOM | 1663 | NE | ARG | A | 211 | 42.110 | 34.563 | 18.631 | 1.00 | 39.40 | 7 | N |
| ATOM | 1664 | CZ | ARG | A | 211 | 42.248 | 33.275 | 18.784 | 1.00 | 41.14 | 6 | C |
| ATOM | 1665 | NH1 | ARG | A | 211 | 42.768 | 32.400 | 17.921 | 1.00 | 37.16 | 7 | N |
| ATOM | 1666 | NH2 | ARG | A | 211 | 41.815 | 32.746 | 19.967 | 1.00 | 38.61 | 7 | N |
| ATOM | 1667 | N | VAL | A | 212 | 40.783 | 41.110 | 18.633 | 1.00 | 40.34 | 7 | N |
| ATOM | 1668 | CA | VAL | A | 212 | 39.777 | 42.134 | 18.516 | 1.00 | 39.19 | 6 | C |
| ATOM | 1669 | C | VAL | A | 212 | 39.596 | 42.458 | 17.043 | 1.00 | 36.20 | 6 | C |
| ATOM | 1670 | O | VAL | A | 212 | 40.612 | 42.645 | 16.384 | 1.00 | 29.64 | 8 | O |
| ATOM | 1671 | CB | VAL | A | 212 | 40.199 | 43.395 | 19.321 | 1.00 | 39.61 | 6 | C |
| ATOM | 1672 | CG1 | VAL | A | 212 | 39.308 | 44.535 | 18.956 | 1.00 | 37.43 | 6 | C |
| ATOM | 1673 | CG2 | VAL | A | 212 | 39.942 | 43.176 | 20.815 | 1.00 | 40.46 | 6 | C |
| ATOM | 1674 | N | ILE | A | 213 | 38.318 | 42.469 | 16.606 | 1.00 | 36.62 | 7 | N |
| ATOM | 1675 | CA | ILE | A | 213 | 37.987 | 42.692 | 15.185 | 1.00 | 36.86 | 6 | C |
| ATOM | 1676 | C | ILE | A | 213 | 32.467 | 44.109 | 15.115 | 1.00 | 38.51 | 6 | C |
| ATOM | 1677 | O | ILE | A | 213 | 36.587 | 44.483 | 15.870 | 1.00 | 40.06 | 8 | O |
| ATOM | 1678 | CB | ILE | A | 213 | 37.096 | 41.587 | 14.582 | 1.00 | 38.12 | 6 | C |
| ATOM | 1679 | CG1 | ILE | A | 213 | 37.841 | 40.249 | 14.689 | 1.00 | 39.53 | 6 | C |
| ATOM | 1680 | CG2 | ILE | A | 213 | 36.862 | 41.753 | 13.070 | 1.00 | 37.90 | 6 | C |
| ATOM | 1681 | CD1 | ILE | A | 213 | 37.200 | 38.921 | 14.505 | 1.00 | 36.98 | 6 | C |
| ATOM | 1682 | N | MET | A | 214 | 38.088 | 44.941 | 14.290 | 1.00 | 40.29 | 7 | N |
| ATOM | 1683 | CA | MET | A | 214 | 37.790 | 46.352 | 14.200 | 1.00 | 40.33 | 6 | C |
| ATOM | 1684 | C | MET | A | 214 | 37.707 | 46.806 | 12.763 | 1.00 | 39.72 | 6 | C |
| ATOM | 1685 | O | MET | A | 214 | 38.183 | 46.193 | 11.779 | 1.00 | 38.00 | 8 | O |
| ATOM | 1686 | CB | MET | A | 214 | 38.987 | 47.047 | 14.909 | 1.00 | 44.70 | 6 | C |
| ATOM | 1687 | CG | MET | A | 214 | 40.326 | 47.090 | 14.203 | 1.00 | 42.63 | 6 | C |
| ATOM | 1688 | SD | MET | A | 214 | 41.567 | 48.132 | 15.031 | 1.00 | 46.50 | 16 | S |
| ATOM | 1689 | CE | MET | A | 214 | 40.764 | 49.713 | 15.182 | 1.00 | 40.28 | 6 | C |
| ATOM | 1690 | N | PRO | A | 215 | 37.030 | 47.925 | 12.544 | 1.00 | 39.84 | 7 | N |
| ATOM | 1691 | CA | PRO | A | 215 | 36.902 | 48.457 | 11.170 | 1.00 | 41.29 | 6 | C |
| ATOM | 1692 | C | PRO | A | 215 | 38.316 | 48.763 | 10.706 | 1.00 | 43.86 | 6 | C |
| ATOM | 1693 | O | PRO | A | 215 | 39.132 | 48.914 | 11.629 | 1.00 | 49.68 | 8 | O |
| ATOM | 1694 | CB | PRO | A | 215 | 36.022 | 49.639 | 11.285 | 1.00 | 37.79 | 6 | C |
| ATOM | 1695 | CG | PRO | A | 215 | 35.702 | 49.857 | 12.691 | 1.00 | 35.62 | 6 | C |
| ATOM | 1696 | CD | PRO | A | 215 | 36.380 | 48.820 | 13.515 | 1.00 | 38.80 | 6 | C |
| ATOM | 1697 | N | ALA | A | 216 | 38.659 | 48.756 | 9.446 | 1.00 | 44.35 | 7 | N |
| ATOM | 1698 | CA | ALA | A | 216 | 40.051 | 49.065 | 9.114 | 1.00 | 46.58 | 6 | C |
| ATOM | 1699 | C | ALA | A | 216 | 40.096 | 50.526 | 8.689 | 1.00 | 48.01 | 6 | C |
| ATOM | 1700 | O | ALA | A | 216 | 39.133 | 50.965 | 8.070 | 1.00 | 46.42 | 8 | O |
| ATOM | 1701 | CB | ALA | A | 216 | 40.565 | 48.326 | 7.888 | 1.00 | 43.48 | 6 | C |
| ATOM | 1702 | N | GLY | A | 217 | 41.192 | 51.241 | 8.904 | 1.00 | 51.30 | 7 | N |
| ATOM | 1703 | CA | GLY | A | 217 | 41.115 | 52.625 | 8.371 | 1.00 | 49.57 | 6 | C |
| ATOM | 1704 | C | GLY | A | 217 | 41.056 | 53.592 | 9.524 | 1.00 | 49.26 | 6 | C |
| ATOM | 1705 | O | GLY | A | 217 | 40.847 | 54.767 | 9.277 | 1.00 | 52.66 | 8 | O |
| ATOM | 1706 | N | TYR | A | 218 | 41.218 | 53.139 | 10.753 | 1.00 | 48.14 | 7 | N |
| ATOM | 1707 | CA | TYR | A | 218 | 41.201 | 53.879 | 11.998 | 1.00 | 49.82 | 6 | C |
| ATOM | 1708 | C | TYR | A | 218 | 42.473 | 53.531 | 12.787 | 1.00 | 52.79 | 6 | C |
| ATOM | 1709 | O | TYR | A | 218 | 42.423 | 52.786 | 13.738 | 1.00 | 54.53 | 8 | O |
| ATOM | 1710 | CB | TYR | A | 218 | 39.929 | 53.561 | 12.822 | 1.00 | 47.34 | 6 | C |
| ATOM | 1711 | CG | TYR | A | 218 | 38.732 | 53.777 | 11.888 | 1.00 | 46.54 | 6 | C |
| ATOM | 1712 | CD1 | TYR | A | 218 | 38.230 | 52.720 | 11.120 | 1.00 | 43.64 | 6 | C |
| ATOM | 1713 | CD2 | TYR | A | 218 | 38.141 | 55.027 | 11.797 | 1.00 | 43.52 | 6 | C |
| ATOM | 1714 | CE1 | TYR | A | 218 | 37.149 | 52.943 | 10.276 | 1.00 | 43.74 | 6 | C |
| ATOM | 1715 | CE2 | TYR | A | 218 | 37.067 | 55.206 | 10.950 | 1.00 | 42.71 | 6 | C |
| ATOM | 1716 | CZ | TYR | A | 218 | 36.565 | 54.191 | 10.191 | 1.00 | 43.20 | 6 | C |
| ATOM | 1717 | OH | TYR | A | 218 | 35.499 | 54.405 | 9.357 | 1.00 | 43.35 | 8 | O |
| ATOM | 1718 | N | PRO | A | 219 | 43.614 | 54.140 | 12.426 | 1.00 | 55.73 | 7 | N |
| ATOM | 1719 | CA | PRO | A | 219 | 44.902 | 53.935 | 13.019 | 1.00 | 55.81 | 6 | C |
| ATOM | 1720 | C | PRO | A | 219 | 45.160 | 54.255 | 14.468 | 1.00 | 56.79 | 6 | C |
| ATOM | 1721 | O | PRO | A | 219 | 45.972 | 53.512 | 15.063 | 1.00 | 58.30 | 8 | O |
| ATOM | 1722 | CB | PRO | A | 219 | 45.901 | 54.744 | 12.185 | 1.00 | 55.80 | 6 | C |
| ATOM | 1723 | CG | PRO | A | 219 | 45.147 | 55.294 | 11.017 | 1.00 | 55.83 | 6 | C |
| ATOM | 1724 | CD | PRO | A | 219 | 43.689 | 55.129 | 11.295 | 1.00 | 55.36 | 6 | C |
| ATOM | 1725 | N | ARG | A | 220 | 44.563 | 55.269 | 15.100 | 1.00 | 57.18 | 7 | N |
| ATOM | 1726 | CA | ARG | A | 220 | 44.941 | 55.451 | 16.517 | 1.00 | 57.81 | 6 | C |
| ATOM | 1727 | C | ARG | A | 220 | 44.292 | 54.302 | 17.262 | 1.00 | 59.10 | 6 | C |
| ATOM | 1728 | O | ARG | A | 220 | 44.997 | 53.572 | 17.974 | 1.00 | 62.71 | 8 | O |
| ATOM | 1729 | CB | ARG | A | 220 | 44.499 | 56.755 | 17.108 | 1.00 | 60.33 | 6 | C |
| ATOM | 1730 | CG | ARG | A | 220 | 45.459 | 57.755 | 16.938 | 0.00 | 25.00 | 6 | C |
| ATOM | 1731 | CD | ARG | A | 220 | 44.915 | 59.019 | 17.565 | 0.00 | 25.00 | 6 | C |
| ATOM | 1732 | NE | ARG | A | 220 | 45.720 | 60.171 | 17.201 | 0.00 | 25.00 | 7 | N |
| ATOM | 1733 | CZ | ARG | A | 220 | 45.496 | 61.403 | 17.626 | 0.00 | 25.00 | 6 | C |
| ATOM | 1734 | NH1 | ARG | A | 220 | 44.480 | 61.649 | 18.439 | 0.00 | 25.00 | 7 | N |
| ATOM | 1735 | NH2 | ARG | A | 220 | 46.291 | 62.389 | 17.235 | 0.00 | 25.00 | 7 | N |
| ATOM | 1736 | N | THR | A | 221 | 42.984 | 54.125 | 17.028 | 1.00 | 55.43 | 7 | N |
| ATOM | 1737 | CA | THR | A | 221 | 42.311 | 53.014 | 17.720 | 1.00 | 50.58 | 6 | C |
| ATOM | 1738 | C | THR | A | 221 | 43.093 | 51.746 | 17.504 | 1.00 | 50.04 | 6 | C |
| ATOM | 1739 | O | THR | A | 221 | 43.182 | 51.002 | 18.526 | 1.00 | 47.68 | 8 | O |
| ATOM | 1740 | CB | THR | A | 221 | 40.839 | 53.010 | 17.271 | 1.00 | 50.88 | 6 | C |
| ATOM | 1741 | OG1 | THR | A | 221 | 40.135 | 54.254 | 17.581 | 1.00 | 50.84 | 8 | O |
| ATOM | 1742 | CG2 | THR | A | 221 | 40.073 | 51.866 | 17.908 | 1.00 | 48.23 | 6 | C |
| ATOM | 1743 | N | ARG | A | 222 | 43.662 | 51.430 | 16.305 | 1.00 | 49.47 | 7 | N |
| ATOM | 1744 | CA | ARG | A | 222 | 44.413 | 50.147 | 16.243 | 1.00 | 49.87 | 6 | C |
| ATOM | 1745 | C | ARG | A | 222 | 45.541 | 50.186 | 17.256 | 1.00 | 50.26 | 6 | C |
| ATOM | 1746 | O | ARG | A | 222 | 45.660 | 49.216 | 17.999 | 1.00 | 51.16 | 8 | O |
| ATOM | 1747 | CB | ARG | A | 222 | 44.948 | 49.709 | 14.900 | 1.00 | 51.29 | 6 | C |
| ATOM | 1748 | CG | ARG | A | 222 | 45.643 | 48.366 | 14.837 | 1.00 | 53.06 | 6 | C |
| ATOM | 1749 | CD | ARG | A | 222 | 46.669 | 47.783 | 13.733 | 0.00 | 25.00 | 6 | C |
| ATOM | 1750 | NE | ARG | A | 222 | 47.574 | 46.698 | 14.032 | 0.00 | 25.00 | 7 | N |
| ATOM | 1751 | CZ | ARG | A | 222 | 48.561 | 46.784 | 14.909 | 0.00 | 25.00 | 6 | C |
| ATOM | 1752 | NH1 | ARG | A | 222 | 48.777 | 47.920 | 15.558 | 0.00 | 25.00 | 7 | N |
| ATOM | 1753 | NH2 | ARG | A | 222 | 49.330 | 45.731 | 15.135 | 0.00 | 25.00 | 7 | N |
| ATOM | 1754 | N | GLU | A | 223 | 46.336 | 51.289 | 17.275 | 1.00 | 51.54 | 7 | N |
| ATOM | 1755 | CA | GLU | A | 223 | 47.411 | 51.301 | 18.283 | 1.00 | 53.43 | 6 | C |
| ATOM | 1756 | C | GLU | A | 223 | 46.832 | 51.298 | 19.689 | 1.00 | 51.80 | 6 | C |
| ATOM | 1757 | O | GLU | A | 223 | 47.170 | 50.466 | 20.546 | 1.00 | 54.40 | 8 | O |
| ATOM | 1758 | CB | GLU | A | 223 | 48.358 | 52.487 | 18.094 | 1.00 | 56.00 | 6 | C |
| ATOM | 1759 | CG | GLU | A | 223 | 49.036 | 52.423 | 16.692 | 0.00 | 25.00 | 6 | C |
| ATOM | 1760 | CD | GLU | A | 223 | 50.012 | 53.553 | 16.505 | 0.00 | 25.00 | 6 | C |
| ATOM | 1761 | OE1 | GLU | A | 223 | 49.926 | 54.538 | 17.265 | 0.00 | 25.00 | 8 | O |
| ATOM | 1762 | OE2 | GLU | A | 223 | 50.861 | 53.450 | 15.599 | 0.00 | 25.00 | 8 | O |
| ATOM | 1763 | N | LYS | A | 224 | 45.886 | 52.179 | 19.966 | 1.00 | 50.35 | 7 | N |
| ATOM | 1764 | CA | LYS | A | 224 | 45.340 | 52.212 | 21.327 | 1.00 | 53.36 | 6 | C |
| ATOM | 1765 | C | LYS | A | 224 | 44.923 | 50.816 | 21.712 | 1.00 | 55.26 | 6 | C |
| ATOM | 1766 | O | LYS | A | 224 | 45.283 | 50.472 | 22.839 | 1.00 | 57.53 | 8 | O |
| ATOM | 1767 | CB | LYS | A | 224 | 44.342 | 53.340 | 21.466 | 1.00 | 54.87 | 6 | C |
| ATOM | 1768 | CG | LYS | A | 224 | 44.582 | 54.743 | 21.028 | 0.00 | 25.00 | 6 | C |
| ATOM | 1769 | CD | LYS | A | 224 | 43.763 | 55.781 | 21.814 | 0.00 | 25.00 | 6 | C |
| ATOM | 1770 | CE | LYS | A | 224 | 43.714 | 57.131 | 21.098 | 0.00 | 25.00 | 6 | C |
| ATOM | 1771 | NZ | LYS | A | 224 | 43.378 | 58.255 | 22.024 | 0.00 | 25.00 | 7 | N |
| ATOM | 1772 | N | ILE | A | 225 | 44.296 | 49.975 | 20.894 | 1.00 | 56.19 | 7 | N |
| ATOM | 1773 | CA | ILE | A | 225 | 43.926 | 48.634 | 21.281 | 1.00 | 56.15 | 6 | C |
| ATOM | 1774 | C | ILE | A | 225 | 45.068 | 47.625 | 21.203 | 1.00 | 55.37 | 6 | C |
| ATOM | 1775 | O | ILE | A | 225 | 45.128 | 46.757 | 22.087 | 1.00 | 54.68 | 8 | O |
| ATOM | 1776 | CB | ILE | A | 225 | 42.798 | 48.059 | 20.397 | 1.00 | 58.42 | 6 | C |
| ATOM | 1777 | CG1 | ILE | A | 225 | 41.690 | 49.080 | 20.154 | 1.00 | 58.53 | 6 | C |
| ATOM | 1778 | CG2 | ILE | A | 225 | 42.233 | 46.749 | 20.982 | 1.00 | 57.48 | 6 | C |
| ATOM | 1779 | CD1 | ILE | A | 225 | 40.467 | 48.500 | 19.477 | 1.00 | 58.69 | 6 | C |
| ATOM | 1780 | N | ALA | A | 226 | 45.893 | 47.746 | 20.170 | 1.00 | 53.98 | 7 | N |
| ATOM | 1781 | CA | ALA | A | 226 | 47.024 | 46.849 | 20.030 | 1.00 | 56.01 | 6 | C |
| ATOM | 1782 | C | ALA | A | 226 | 47.956 | 46.906 | 21.226 | 1.00 | 59.14 | 6 | C |
| ATOM | 1783 | O | ALA | A | 226 | 48.452 | 45.821 | 21.565 | 1.00 | 59.23 | 8 | O |
| ATOM | 1784 | CB | ALA | A | 226 | 47.788 | 47.214 | 18.767 | 1.00 | 57.58 | 6 | C |
| ATOM | 1785 | N | ARG | A | 227 | 48.173 | 48.063 | 21.904 | 1.00 | 61.13 | 7 | N |
| ATOM | 1786 | CA | ARG | A | 227 | 49.026 | 48.117 | 23.075 | 1.00 | 63.62 | 6 | C |
| ATOM | 1787 | C | ARG | A | 227 | 48.611 | 47.338 | 24.314 | 1.00 | 64.16 | 6 | C |
| ATOM | 1788 | O | ARG | A | 227 | 49.437 | 46.972 | 25.176 | 1.00 | 66.16 | 8 | O |
| ATOM | 1789 | CB | ARG | A | 227 | 49.152 | 49.589 | 23.529 | 1.00 | 65.62 | 6 | C |
| ATOM | 1790 | CG | ARG | A | 227 | 50.119 | 50.424 | 22.618 | 0.00 | 25.00 | 6 | C |
| ATOM | 1791 | CD | ARG | A | 227 | 50.137 | 51.866 | 23.123 | 0.00 | 25.00 | 6 | C |
| ATOM | 1792 | NE | ARG | A | 227 | 50.816 | 52.764 | 22.193 | 0.00 | 25.00 | 7 | N |
| ATOM | 1793 | CZ | ARG | A | 227 | 50.977 | 54.075 | 22.377 | 0.00 | 25.00 | 6 | C |
| ATOM | 1794 | NH1 | ARG | A | 227 | 50.505 | 54.662 | 23.467 | 0.00 | 25.00 | 7 | N |
| ATOM | 1795 | NH2 | ARG | A | 227 | 51.615 | 54.796 | 21.461 | 0.00 | 25.00 | 7 | N |
| ATOM | 1796 | N | LEU | A | 228 | 47.332 | 47.023 | 24.468 | 1.00 | 63.40 | 7 | N |
| ATOM | 1797 | CA | LEU | A | 228 | 46.800 | 46.309 | 25.616 | 1.00 | 65.63 | 6 | C |
| ATOM | 1798 | C | LEU | A | 228 | 47.119 | 44.824 | 25.539 | 1.00 | 66.14 | 6 | C |
| ATOM | 1799 | O | LEU | A | 228 | 46.831 | 44.063 | 26.479 | 1.00 | 67.67 | 8 | O |
| ATOM | 1800 | CB | LEU | A | 228 | 45.308 | 46.555 | 25.811 | 1.00 | 65.36 | 6 | C |
| ATOM | 1801 | CG | LEU | A | 228 | 44.910 | 48.044 | 25.837 | 1.00 | 66.97 | 6 | C |
| ATOM | 1802 | CD1 | LEU | A | 228 | 44.306 | 48.481 | 24.516 | 1.00 | 68.89 | 6 | C |
| ATOM | 1803 | CD2 | LEU | A | 228 | 43.905 | 48.312 | 26.950 | 1.00 | 66.75 | 6 | C |
| ATOM | 1804 | N | GLY | A | 229 | 47.740 | 44.440 | 24.418 | 1.00 | 65.41 | 7 | N |
| ATOM | 1805 | CA | GLY | A | 229 | 48.124 | 43.030 | 24.260 | 1.00 | 65.53 | 6 | C |
| ATOM | 1806 | C | GLY | A | 229 | 47.176 | 42.184 | 23.423 | 1.00 | 64.09 | 6 | C |
| ATOM | 1807 | O | GLY | A | 229 | 47.252 | 40.953 | 23.438 | 1.00 | 65.55 | 8 | O |
| ATOM | 1808 | N | TYR | A | 230 | 46.267 | 42.770 | 22.656 | 1.00 | 60.84 | 7 | N |
| ATOM | 1809 | CA | TYR | A | 230 | 45.295 | 42.028 | 21.903 | 1.00 | 59.56 | 6 | C |
| ATOM | 1810 | C | TYR | A | 230 | 45.682 | 41.704 | 20.475 | 1.00 | 58.97 | 6 | C |
| ATOM | 1811 | O | TYR | A | 230 | 46.143 | 42.642 | 19.791 | 1.00 | 61.04 | 8 | O |
| ATOM | 1812 | CB | TYR | A | 230 | 44.001 | 42.830 | 21.752 | 1.00 | 59.91 | 6 | C |
| ATOM | 1813 | CG | TYR | A | 230 | 43.187 | 43.068 | 22.993 | 1.00 | 59.58 | 6 | C |
| ATOM | 1814 | CD1 | TYR | A | 230 | 42.594 | 42.015 | 23.660 | 1.00 | 60.51 | 6 | C |
| ATOM | 1815 | CD2 | TYR | A | 230 | 43.010 | 44.340 | 23.496 | 1.00 | 59.34 | 6 | C |
| ATOM | 1816 | CE1 | TYR | A | 230 | 41.834 | 42.218 | 24.813 | 1.00 | 60.26 | 6 | C |
| ATOM | 1817 | CE2 | TYR | A | 230 | 42.253 | 44.542 | 24.646 | 1.00 | 59.88 | 6 | C |
| ATOM | 1818 | CZ | TYR | A | 230 | 41.670 | 43.484 | 25.296 | 1.00 | 58.58 | 6 | C |
| ATOM | 1819 | OH | TYR | A | 230 | 40.938 | 43.735 | 26.419 | 1.00 | 59.32 | 8 | O |
| ATOM | 1820 | N | ARG | A | 231 | 45.450 | 40.459 | 20.016 | 1.00 | 54.96 | 7 | N |
| ATOM | 1821 | CA | ARG | A | 231 | 45.865 | 40.304 | 18.617 | 1.00 | 54.15 | 6 | C |
| ATOM | 1822 | C | ARG | A | 231 | 44.813 | 41.052 | 17.794 | 1.00 | 50.91 | 6 | C |
| ATOM | 1823 | O | ARG | A | 231 | 43.708 | 40.489 | 17.728 | 1.00 | 53.46 | 8 | O |
| ATOM | 1824 | CB | ARG | A | 231 | 46.004 | 38.908 | 18.034 | 1.00 | 56.43 | 6 | C |
| ATOM | 1825 | CG | ARG | A | 231 | 45.930 | 38.886 | 16.666 | 0.00 | 25.00 | 6 | C |
| ATOM | 1826 | CD | ARG | A | 231 | 46.207 | 37.382 | 16.461 | 0.00 | 25.00 | 6 | C |
| ATOM | 1827 | NE | ARG | A | 231 | 47.397 | 37.103 | 17.282 | 0.00 | 25.00 | 7 | N |
| ATOM | 1828 | CZ | ARG | A | 231 | 47.860 | 35.910 | 17.610 | 0.00 | 25.00 | 6 | C |
| ATOM | 1829 | NH1 | ARG | A | 231 | 47.214 | 34.830 | 17.163 | 0.00 | 25.00 | 7 | N |
| ATOM | 1830 | NH2 | ARG | A | 231 | 48.963 | 35.851 | 18.360 | 0.00 | 25.00 | 7 | N |
| ATOM | 1831 | N | VAL | A | 232 | 45.111 | 42.187 | 17.211 | 1.00 | 44.43 | 7 | N |
| ATOM | 1832 | CA | VAL | A | 232 | 44.108 | 42.901 | 16.474 | 1.00 | 42.47 | 6 | C |
| ATOM | 1833 | C | VAL | A | 232 | 43.958 | 42.460 | 15.032 | 1.00 | 43.54 | 6 | C |
| ATOM | 1834 | O | VAL | A | 232 | 44.988 | 42.214 | 14.396 | 1.00 | 46.25 | 8 | O |
| ATOM | 1835 | CB | VAL | A | 232 | 44.517 | 44.388 | 16.488 | 1.00 | 42.47 | 6 | C |
| ATOM | 1836 | CG1 | VAL | A | 232 | 43.543 | 45.305 | 15.780 | 1.00 | 41.72 | 6 | C |
| ATOM | 1837 | CG2 | VAL | A | 232 | 44.716 | 44.757 | 17.957 | 1.00 | 43.14 | 6 | C |
| ATOM | 1838 | N | ILE | A | 233 | 42.739 | 42.388 | 14.500 | 1.00 | 40.02 | 7 | N |
| ATOM | 1839 | CA | ILE | A | 233 | 42.510 | 42.012 | 13.113 | 1.00 | 36.26 | 6 | C |
| ATOM | 1840 | C | ILE | A | 233 | 41.665 | 43.090 | 12.464 | 1.00 | 38.47 | 6 | C |
| ATOM | 1841 | O | ILE | A | 233 | 40.659 | 43.416 | 13.134 | 1.00 | 43.21 | 8 | O |
| ATOM | 1842 | CB | ILE | A | 233 | 41.660 | 40.751 | 12.968 | 1.00 | 34.33 | 6 | C |
| ATOM | 1843 | CG1 | ILE | A | 233 | 42.016 | 39.642 | 13.973 | 1.00 | 30.26 | 6 | C |
| ATOM | 1844 | CG2 | ILE | A | 233 | 41.740 | 40.379 | 11.503 | 1.00 | 32.65 | 6 | C |
| ATOM | 1845 | CD1 | ILE | A | 233 | 41.706 | 38.261 | 13.420 | 1.00 | 25.33 | 6 | C |
| ATOM | 1846 | N | GLU | A | 234 | 41.986 | 43.694 | 11.367 | 1.00 | 39.81 | 7 | N |
| ATOM | 1847 | CA | GLU | A | 234 | 41.212 | 44.751 | 10.810 | 1.00 | 42.23 | 6 | C |
| ATOM | 1848 | C | GLU | A | 234 | 40.439 | 44.306 | 9.571 | 1.00 | 42.51 | 6 | C |
| ATOM | 1849 | O | GLU | A | 234 | 41.010 | 43.701 | 8.651 | 1.00 | 42.80 | 8 | O |
| ATOM | 1850 | CB | GLU | A | 234 | 42.014 | 45.942 | 10.258 | 1.00 | 45.69 | 6 | C |
| ATOM | 1851 | CG | GLU | A | 234 | 43.050 | 46.367 | 11.276 | 1.00 | 51.51 | 6 | C |
| ATOM | 1852 | CD | GLU | A | 234 | 43.939 | 47.450 | 10.658 | 1.00 | 54.18 | 6 | C |
| ATOM | 1853 | OE1 | GLU | A | 234 | 43.510 | 48.436 | 10.065 | 1.00 | 54.73 | 8 | O |
| ATOM | 1854 | OE2 | GLU | A | 234 | 45.130 | 47.136 | 10.872 | 1.00 | 60.36 | 8 | O |
| ATOM | 1855 | N | VAL | A | 235 | 39.150 | 44.675 | 9.549 | 1.00 | 38.95 | 7 | N |
| ATOM | 1856 | CA | VAL | A | 235 | 38.315 | 44.308 | 8.402 | 1.00 | 32.27 | 6 | C |
| ATOM | 1857 | C | VAL | A | 235 | 37.584 | 45.510 | 7.845 | 1.00 | 32.84 | 6 | C |
| ATOM | 1858 | O | VAL | A | 235 | 36.967 | 46.319 | 8.571 | 1.00 | 39.63 | 8 | O |
| ATOM | 1859 | CB | VAL | A | 235 | 37.286 | 43.250 | 8.815 | 1.00 | 29.09 | 6 | C |
| ATOM | 1860 | CG1 | VAL | A | 235 | 36.242 | 43.119 | 7.739 | 1.00 | 30.71 | 6 | C |
| ATOM | 1861 | CG2 | VAL | A | 235 | 38.018 | 41.910 | 8.971 | 1.00 | 26.44 | 6 | C |
| ATOM | 1862 | N | ASP | A | 236 | 37.576 | 45.748 | 6.595 | 1.00 | 29.05 | 7 | N |
| ATOM | 1863 | CA | ASP | A | 236 | 37.016 | 46.831 | 5.879 | 1.00 | 29.93 | 6 | C |
| ATOM | 1864 | C | ASP | A | 236 | 35.522 | 46.570 | 5.845 | 1.00 | 32.97 | 6 | C |
| ATOM | 1865 | O | ASP | A | 236 | 34.997 | 45.701 | 5.131 | 1.00 | 37.80 | 8 | O |
| ATOM | 1866 | CB | ASP | A | 236 | 37.589 | 47.067 | 4.471 | 1.00 | 30.16 | 6 | C |
| ATOM | 1867 | CG | ASP | A | 236 | 37.108 | 48.346 | 3.808 | 1.00 | 34.70 | 6 | C |
| ATOM | 1868 | OD1 | ASP | A | 236 | 36.209 | 49.041 | 4.409 | 1.00 | 39.80 | 8 | O |
| ATOM | 1869 | OD2 | ASP | A | 236 | 37.496 | 48.826 | 2.701 | 1.00 | 33.27 | 8 | O |
| ATOM | 1870 | N | THR | A | 237 | 34.741 | 47.328 | 6.584 | 1.00 | 32.66 | 7 | N |
| ATOM | 1871 | CA | THR | A | 237 | 33.335 | 47.247 | 6.674 | 1.00 | 33.54 | 6 | C |
| ATOM | 1872 | C | THR | A | 237 | 32.742 | 48.532 | 6.144 | 1.00 | 35.71 | 6 | C |
| ATOM | 1873 | O | THR | A | 237 | 31.618 | 48.915 | 6.390 | 1.00 | 37.77 | 8 | O |
| ATOM | 1874 | CB | THR | A | 237 | 32.871 | 46.988 | 8.092 | 1.00 | 34.57 | 6 | C |
| ATOM | 1875 | OG1 | THR | A | 237 | 33.186 | 48.191 | 8.773 | 1.00 | 37.21 | 8 | O |
| ATOM | 1876 | CG2 | THR | A | 237 | 33.563 | 45.759 | 8.685 | 1.00 | 33.69 | 6 | C |
| ATOM | 1877 | N | SER | A | 238 | 33.464 | 49.215 | 5.290 | 1.00 | 36.98 | 7 | N |
| ATOM | 1878 | CA | SER | A | 238 | 33.024 | 50.483 | 4.739 | 1.00 | 37.00 | 6 | C |
| ATOM | 1879 | C | SER | A | 238 | 31.648 | 50.373 | 4.196 | 1.00 | 39.28 | 6 | C |
| ATOM | 1880 | O | SER | A | 238 | 30.950 | 51.346 | 4.509 | 1.00 | 45.03 | 8 | O |
| ATOM | 1881 | CB | SER | A | 238 | 33.899 | 50.997 | 3.604 | 1.00 | 37.13 | 6 | C |
| ATOM | 1882 | OG | SER | A | 238 | 33.618 | 50.300 | 2.367 | 1.00 | 40.52 | 8 | O |
| ATOM | 1883 | N | GLU | A | 239 | 31.242 | 49.352 | 3.428 | 1.00 | 37.51 | 7 | N |
| ATOM | 1884 | CA | GLU | A | 239 | 29.827 | 49.466 | 3.028 | 1.00 | 35.11 | 6 | C |
| ATOM | 1885 | C | GLU | A | 239 | 28.885 | 49.210 | 4.197 | 1.00 | 34.31 | 6 | C |
| ATOM | 1886 | O | GLU | A | 239 | 27.694 | 49.497 | 4.037 | 1.00 | 37.23 | 8 | O |
| ATOM | 1887 | CB | GLU | A | 239 | 29.637 | 48.453 | 1.902 | 1.00 | 35.91 | 6 | C |
| ATOM | 1888 | CG | GLU | A | 239 | 30.673 | 48.554 | 0.825 | 1.00 | 37.60 | 6 | C |
| ATOM | 1889 | CD | GLU | A | 239 | 30.455 | 49.892 | 0.130 | 1.00 | 40.45 | 6 | C |
| ATOM | 1890 | OE1 | GLU | A | 239 | 29.345 | 50.192 | −0.347 | 1.00 | 42.01 | 8 | O |
| ATOM | 1891 | OE2 | GLU | A | 239 | 31.406 | 50.681 | 0.052 | 1.00 | 45.12 | 8 | O |
| ATOM | 1892 | N | TYR | A | 240 | 29.129 | 48.680 | 5.364 | 1.00 | 31.35 | 7 | N |
| ATOM | 1893 | CA | TYR | A | 240 | 28.242 | 48.478 | 6.470 | 1.00 | 31.70 | 6 | C |
| ATOM | 1894 | C | TYR | A | 240 | 28.063 | 49.825 | 7.220 | 1.00 | 35.64 | 6 | C |
| ATOM | 1895 | O | TYR | A | 240 | 26.994 | 50.147 | 7.798 | 1.00 | 37.04 | 8 | O |
| ATOM | 1896 | CB | TYR | A | 240 | 28.746 | 47.314 | 7.401 | 1.00 | 27.47 | 6 | C |
| ATOM | 1897 | CG | TYR | A | 240 | 28.527 | 46.099 | 6.499 | 1.00 | 28.07 | 6 | C |
| ATOM | 1898 | CD1 | TYR | A | 240 | 29.590 | 45.548 | 5.794 | 1.00 | 26.26 | 6 | C |
| ATOM | 1899 | CD2 | TYR | A | 240 | 27.237 | 45.537 | 6.368 | 1.00 | 23.44 | 6 | C |
| ATOM | 1900 | CE1 | TYR | A | 240 | 29.409 | 44.453 | 4.965 | 1.00 | 23.22 | 6 | C |
| ATOM | 1901 | CE2 | TYR | A | 240 | 27.089 | 44.457 | 5.543 | 1.00 | 20.35 | 6 | C |
| ATOM | 1902 | CZ | TYR | A | 240 | 28.167 | 43.928 | 4.852 | 1.00 | 22.01 | 6 | C |
| ATOM | 1903 | OH | TYR | A | 240 | 27.936 | 42.833 | 4.038 | 1.00 | 19.69 | 8 | O |
| ATOM | 1904 | N | ARG | A | 241 | 29.097 | 50.609 | 7.207 | 1.00 | 34.06 | 7 | N |
| ATOM | 1905 | CA | ARG | A | 241 | 29.134 | 51.900 | 7.870 | 1.00 | 40.82 | 6 | C |
| ATOM | 1906 | C | ARG | A | 241 | 28.056 | 52.812 | 7.285 | 1.00 | 43.73 | 6 | C |
| ATOM | 1907 | O | ARG | A | 241 | 27.202 | 53.445 | 7.922 | 1.00 | 41.78 | 8 | O |
| ATOM | 1908 | CB | ARG | A | 241 | 30.540 | 52.485 | 7.601 | 1.00 | 38.65 | 6 | C |
| ATOM | 1909 | CG | ARG | A | 241 | 30.469 | 53.944 | 8.036 | 1.00 | 41.33 | 6 | C |
| ATOM | 1910 | CD | ARG | A | 241 | 31.850 | 54.429 | 8.445 | 1.00 | 42.66 | 6 | C |
| ATOM | 1911 | NE | ARG | A | 241 | 31.795 | 55.804 | 8.846 | 1.00 | 50.06 | 7 | N |
| ATOM | 1912 | CZ | ARG | A | 241 | 32.088 | 56.825 | 8.037 | 1.00 | 52.28 | 6 | C |
| ATOM | 1913 | NH1 | ARG | A | 241 | 32.446 | 56.555 | 6.793 | 1.00 | 51.84 | 7 | N |
| ATOM | 1914 | NH2 | ARG | A | 241 | 32.040 | 58.095 | 8.410 | 1.00 | 53.84 | 7 | N |
| ATOM | 1915 | N | LYS | A | 242 | 27.994 | 52.750 | 5.925 | 1.00 | 45.81 | 7 | N |
| ATOM | 1916 | CA | LYS | A | 242 | 27.021 | 53.507 | 5.188 | 1.00 | 45.61 | 6 | C |
| ATOM | 1917 | C | LYS | A | 242 | 25.622 | 53.187 | 5.703 | 1.00 | 44.16 | 6 | C |
| ATOM | 1918 | O | LYS | A | 242 | 24.757 | 54.046 | 5.417 | 1.00 | 45.96 | 8 | O |
| ATOM | 1919 | CB | LYS | A | 242 | 27.024 | 53.359 | 3.668 | 1.00 | 45.65 | 6 | C |
| ATOM | 1920 | CG | LYS | A | 242 | 28.386 | 53.678 | 3.069 | 1.00 | 45.89 | 6 | C |
| ATOM | 1921 | CD | LYS | A | 242 | 28.341 | 53.500 | 1.566 | 1.00 | 44.34 | 6 | C |
| ATOM | 1922 | CE | LYS | A | 242 | 29.725 | 53.173 | 1.022 | 1.00 | 43.84 | 6 | C |
| ATOM | 1923 | NZ | LYS | A | 242 | 29.513 | 52.924 | −0.465 | 1.00 | 49.40 | 7 | N |
| ATOM | 1924 | N | ILE | A | 243 | 25.378 | 52.116 | 6.378 | 1.00 | 39.32 | 7 | N |
| ATOM | 1925 | CA | ILE | A | 243 | 24.011 | 51.912 | 6.871 | 1.00 | 39.57 | 6 | C |
| ATOM | 1926 | C | ILE | A | 243 | 24.070 | 51.775 | 8.394 | 1.00 | 41.19 | 6 | C |
| ATOM | 1927 | O | ILE | A | 243 | 23.255 | 51.106 | 9.018 | 1.00 | 41.14 | 8 | O |
| ATOM | 1928 | CB | ILE | A | 243 | 23.352 | 50.758 | 6.106 | 1.00 | 37.97 | 6 | C |
| ATOM | 1929 | CG1 | ILE | A | 243 | 24.253 | 49.537 | 5.977 | 1.00 | 36.16 | 6 | C |
| ATOM | 1930 | CG2 | ILE | A | 243 | 22.947 | 51.150 | 4.689 | 1.00 | 36.16 | 6 | C |
| ATOM | 1931 | CD1 | ILE | A | 243 | 23.627 | 48.377 | 5.236 | 1.00 | 33.76 | 6 | C |
| ATOM | 1932 | N | ASP | A | 244 | 25.060 | 52.425 | 9.003 | 1.00 | 43.35 | 7 | N |
| ATOM | 1933 | CA | ASP | A | 244 | 25.304 | 52.424 | 10.436 | 1.00 | 46.12 | 6 | C |
| ATOM | 1934 | C | ASP | A | 244 | 25.413 | 51.085 | 11.133 | 1.00 | 45.02 | 6 | C |
| ATOM | 1935 | O | ASP | A | 244 | 24.770 | 50.774 | 12.136 | 1.00 | 47.32 | 8 | O |
| ATOM | 1936 | CB | ASP | A | 244 | 24.240 | 53.315 | 11.103 | 1.00 | 51.54 | 6 | C |
| ATOM | 1937 | CG | ASP | A | 244 | 24.391 | 54.776 | 10.722 | 1.00 | 57.18 | 6 | C |
| ATOM | 1938 | OD1 | ASP | A | 244 | 25.374 | 55.425 | 10.244 | 1.00 | 60.02 | 8 | O |
| ATOM | 1939 | OD2 | ASP | A | 244 | 23.316 | 55.377 | 10.946 | 1.00 | 61.67 | 8 | O |
| ATOM | 1940 | N | GLY | A | 245 | 26.262 | 50.184 | 10.635 | 1.00 | 43.80 | 7 | N |
| ATOM | 1941 | CA | GLY | A | 245 | 26.499 | 48.851 | 11.232 | 1.00 | 35.29 | 6 | C |
| ATOM | 1942 | C | GLY | A | 245 | 28.005 | 48.616 | 11.062 | 1.00 | 32.59 | 6 | C |
| ATOM | 1943 | O | GLY | A | 245 | 28.715 | 49.192 | 10.254 | 1.00 | 30.63 | 8 | O |
| ATOM | 1944 | N | GLY | A | 246 | 28.565 | 47.705 | 11.778 | 1.00 | 33.22 | 7 | N |
| ATOM | 1945 | CA | GLY | A | 246 | 29.937 | 47.350 | 11.799 | 1.00 | 30.36 | 6 | C |
| ATOM | 1946 | C | GLY | A | 246 | 30.123 | 46.035 | 12.478 | 1.00 | 31.65 | 6 | C |
| ATOM | 1947 | O | GLY | A | 246 | 29.079 | 45.357 | 12.639 | 1.00 | 39.23 | 8 | O |
| ATOM | 1948 | N | VAL | A | 247 | 31.324 | 45.721 | 12.826 | 1.00 | 27.74 | 7 | N |
| ATOM | 1949 | CA | VAL | A | 247 | 31.715 | 44.442 | 13.326 | 1.00 | 30.79 | 6 | C |
| ATOM | 1950 | C | VAL | A | 247 | 30.906 | 43.934 | 14.469 | 1.00 | 31.67 | 6 | C |
| ATOM | 1951 | O | VAL | A | 247 | 30.432 | 42.789 | 14.226 | 1.00 | 36.18 | 8 | O |
| ATOM | 1952 | CB | VAL | A | 247 | 33.254 | 44.308 | 13.499 | 1.00 | 33.23 | 6 | C |
| ATOM | 1953 | CG1 | VAL | A | 247 | 33.838 | 44.731 | 12.114 | 1.00 | 28.99 | 6 | C |
| ATOM | 1954 | CG2 | VAL | A | 247 | 33.699 | 45.154 | 14.691 | 1.00 | 29.14 | 6 | C |
| ATOM | 1955 | N | SER | A | 248 | 30.575 | 44.558 | 15.536 | 1.00 | 31.24 | 7 | N |
| ATOM | 1956 | CA | SER | A | 248 | 29.713 | 43.853 | 16.503 | 1.00 | 32.91 | 6 | C |
| ATOM | 1957 | C | SER | A | 248 | 28.346 | 43.551 | 15.948 | 1.00 | 30.13 | 6 | C |
| ATOM | 1958 | O | SER | A | 248 | 27.669 | 42.597 | 16.391 | 1.00 | 29.30 | 8 | O |
| ATOM | 1959 | CB | SER | A | 248 | 29.570 | 44.795 | 17.724 | 1.00 | 33.72 | 6 | C |
| ATOM | 1960 | OG | SER | A | 248 | 28.169 | 44.870 | 18.039 | 1.00 | 35.59 | 8 | O |
| ATOM | 1961 | N | CYS | A | 249 | 27.861 | 44.333 | 14.995 | 1.00 | 29.88 | 7 | N |
| ATOM | 1962 | CA | CYS | A | 249 | 26.442 | 44.080 | 14.612 | 1.00 | 33.92 | 6 | C |
| ATOM | 1963 | C | CYS | A | 249 | 26.224 | 42.858 | 13.749 | 1.00 | 32.35 | 6 | C |
| ATOM | 1964 | O | CYS | A | 249 | 25.122 | 42.338 | 13.570 | 1.00 | 32.83 | 8 | O |
| ATOM | 1965 | CB | CYS | A | 249 | 25.687 | 45.208 | 13.881 | 1.00 | 32.82 | 6 | C |
| ATOM | 1966 | SG | CYS | A | 249 | 26.132 | 46.893 | 14.308 | 1.00 | 40.88 | 16 | S |
| ATOM | 1967 | N | MET | A | 250 | 27.293 | 42.417 | 13.097 | 1.00 | 31.22 | 7 | N |
| ATOM | 1968 | CA | MET | A | 250 | 27.207 | 41.274 | 12.205 | 1.00 | 27.58 | 6 | C |
| ATOM | 1969 | C | MET | A | 250 | 27.377 | 39.909 | 12.819 | 1.00 | 26.29 | 6 | C |
| ATOM | 1970 | O | MET | A | 250 | 27.430 | 38.972 | 11.988 | 1.00 | 24.48 | 8 | O |
| ATOM | 1971 | CB | MET | A | 250 | 28.184 | 41.525 | 11.034 | 1.00 | 27.22 | 6 | C |
| ATOM | 1972 | CG | MET | A | 250 | 27.753 | 42.808 | 10.312 | 1.00 | 31.32 | 6 | C |
| ATOM | 1973 | SD | MET | A | 250 | 28.616 | 43.011 | 8.719 | 1.00 | 37.29 | 16 | S |
| ATOM | 1974 | CE | MET | A | 250 | 30.115 | 43.823 | 9.396 | 1.00 | 33.74 | 6 | C |
| ATOM | 1975 | N | SER | A | 251 | 27.414 | 39.657 | 14.101 | 1.00 | 24.51 | 7 | N |
| ATOM | 1976 | CA | SER | A | 251 | 27.528 | 38.309 | 14.637 | 1.00 | 25.08 | 6 | C |
| ATOM | 1977 | C | SER | A | 251 | 26.927 | 38.231 | 16.062 | 1.00 | 25.88 | 6 | C |
| ATOM | 1978 | O | SER | A | 251 | 26.712 | 39.297 | 16.698 | 1.00 | 23.60 | 8 | O |
| ATOM | 1979 | CB | SER | A | 251 | 28.937 | 37.787 | 14.580 | 1.00 | 24.88 | 6 | C |
| ATOM | 1980 | OG | SER | A | 251 | 29.971 | 38.436 | 15.303 | 1.00 | 27.27 | 8 | O |
| ATOM | 1981 | N | LEU | A | 252 | 26.642 | 36.999 | 16.453 | 1.00 | 22.94 | 7 | N |
| ATOM | 1982 | CA | LEU | A | 252 | 26.198 | 36.712 | 17.800 | 1.00 | 27.75 | 6 | C |
| ATOM | 1983 | C | LEU | A | 252 | 27.375 | 35.915 | 18.409 | 1.00 | 29.48 | 6 | C |
| ATOM | 1984 | O | LEU | A | 252 | 27.850 | 34.990 | 17.703 | 1.00 | 30.89 | 8 | O |
| ATOM | 1985 | CB | LEU | A | 252 | 24.984 | 35.803 | 17.783 | 1.00 | 27.94 | 6 | C |
| ATOM | 1986 | CG | LEU | A | 252 | 23.563 | 36.254 | 17.823 | 1.00 | 29.02 | 6 | C |
| ATOM | 1987 | CD1 | LEU | A | 252 | 23.339 | 37.696 | 18.234 | 1.00 | 23.62 | 6 | C |
| ATOM | 1988 | CD2 | LEU | A | 252 | 22.690 | 35.798 | 16.622 | 1.00 | 25.49 | 6 | C |
| ATOM | 1989 | N | ARG | A | 253 | 27.903 | 36.171 | 19.562 | 1.00 | 31.90 | 7 | N |
| ATOM | 1990 | CA | ARG | A | 253 | 29.031 | 35.441 | 20.175 | 1.00 | 33.27 | 6 | C |
| ATOM | 1991 | C | ARG | A | 253 | 28.538 | 34.811 | 21.473 | 1.00 | 34.40 | 6 | C |
| ATOM | 1992 | O | ARG | A | 253 | 27.952 | 35.708 | 22.162 | 1.00 | 35.67 | 8 | O |
| ATOM | 1993 | CB | ARG | A | 253 | 30.157 | 36.394 | 20.479 | 1.00 | 33.83 | 6 | C |
| ATOM | 1994 | CG | ARG | A | 253 | 30.611 | 37.091 | 19.222 | 1.00 | 38.64 | 6 | C |
| ATOM | 1995 | CD | ARG | A | 253 | 31.173 | 38.490 | 19.258 | 1.00 | 44.75 | 6 | C |
| ATOM | 1996 | NE | ARG | A | 253 | 30.460 | 39.685 | 19.713 | 1.00 | 42.66 | 7 | N |
| ATOM | 1997 | CZ | ARG | A | 253 | 29.616 | 40.301 | 18.909 | 1.00 | 45.74 | 6 | C |
| ATOM | 1998 | NH1 | ARG | A | 253 | 29.001 | 41.388 | 19.388 | 1.00 | 49.52 | 7 | N |
| ATOM | 1999 | NH2 | ARG | A | 253 | 29.322 | 39.936 | 17.651 | 1.00 | 43.04 | 7 | N |
| ATOM | 2000 | N | PHE | A | 254 | 28.723 | 33.518 | 21.759 | 1.00 | 29.15 | 7 | N |
| ATOM | 2001 | CA | PHE | A | 254 | 28.253 | 32.992 | 23.026 | 1.00 | 29.52 | 6 | C |
| ATOM | 2002 | C | PHE | A | 254 | 28.800 | 31.565 | 23.515 | 1.00 | 28.81 | 6 | C |
| ATOM | 2003 | O | PHE | A | 254 | 29.705 | 31.352 | 22.632 | 1.00 | 34.77 | 8 | O |
| ATOM | 2004 | CB | PHE | A | 254 | 26.811 | 32.764 | 22.929 | 1.00 | 26.68 | 6 | C |
| ATOM | 2005 | CG | PHE | A | 254 | 26.290 | 31.847 | 21.871 | 1.00 | 30.72 | 6 | C |
| ATOM | 2006 | CD1 | PHE | A | 254 | 26.280 | 30.478 | 22.007 | 1.00 | 30.11 | 6 | C |
| ATOM | 2007 | CD2 | PHE | A | 254 | 25.767 | 32.336 | 20.683 | 1.00 | 29.32 | 6 | C |
| ATOM | 2008 | CE1 | PHE | A | 254 | 25.783 | 29.634 | 21.024 | 1.00 | 29.34 | 6 | C |
| ATOM | 2009 | CE2 | PHE | A | 254 | 25.279 | 31.528 | 19.712 | 1.00 | 26.03 | 6 | C |
| ATOM | 2010 | CZ | PHE | A | 254 | 25.286 | 30.162 | 19.880 | 1.00 | 29.57 | 6 | C |
| TER | | | | | | | | | | | | |
| ATOM | 1 | O1 | HOH | W | 1 | 28.025 | 28.390 | 15.602 | 1.00 | 42.31 | 8 | O |
| ATOM | 2 | O1 | HOH | W | 2 | 26.268 | 38.572 | 9.634 | 1.00 | 18.83 | 8 | O |
| ATOM | 3 | O1 | HOH | W | 3 | 20.131 | 38.583 | 4.610 | 1.00 | 26.79 | 8 | O |
| ATOM | 4 | O1 | HOH | W | 4 | 20.970 | 41.393 | 4.696 | 1.00 | 37.64 | 8 | O |
| ATOM | 5 | O1 | HOH | W | 5 | 40.024 | 29.325 | 14.898 | 1.00 | 46.73 | 8 | O |
| ATOM | 6 | O1 | HOH | W | 6 | 32.991 | 23.495 | 17.663 | 1.00 | 59.22 | 8 | O |
| ATOM | 7 | O1 | HOH | W | 7 | 15.840 | 43.990 | 7.507 | 1.00 | 36.79 | 8 | O |
| ATOM | 8 | O1 | HOH | W | 8 | 18.953 | 39.159 | 11.374 | 1.00 | 31.57 | 8 | O |
| ATOM | 9 | O1 | HOH | W | 9 | 24.174 | 38.042 | 22.193 | 1.00 | 28.52 | 8 | O |
| ATOM | 10 | O1 | HOH | W | 10 | 27.171 | 35.756 | 25.175 | 1.00 | 43.63 | 8 | O |
| ATOM | 11 | O1 | HOH | W | 11 | 14.947 | 46.648 | 7.221 | 1.00 | 73.87 | 8 | O |
| ATOM | 12 | O1 | HOH | W | 12 | 9.614 | 42.318 | 10.157 | 1.00 | 66.39 | 8 | O |
| ATOM | 13 | O1 | HOH | W | 13 | 17.391 | 30.066 | 12.079 | 1.00 | 22.71 | 8 | O |
| ATOM | 14 | O1 | HOH | W | 14 | 13.770 | 30.141 | 12.628 | 1.00 | 47.82 | 8 | O |
| ATOM | 15 | O1 | HOH | W | 15 | 10.140 | 34.531 | 27.487 | 1.00 | 48.18 | 8 | O |
| ATOM | 16 | O1 | HOH | W | 16 | 10.347 | 38.810 | 20.645 | 1.00 | 61.45 | 8 | O |
| ATOM | 17 | O1 | HOH | W | 17 | 4.605 | 40.349 | 21.239 | 1.00 | 58.90 | 8 | O |
| ATOM | 18 | O1 | HOH | W | 18 | 24.452 | 42.317 | 22.059 | 1.00 | 27.18 | 8 | O |
| ATOM | 19 | O1 | HOH | W | 19 | 27.836 | 42.629 | 24.467 | 1.00 | 53.13 | 8 | O |
| ATOM | 20 | O1 | HOH | W | 20 | 30.376 | 42.435 | 25.640 | 1.00 | 39.14 | 8 | O |
| ATOM | 21 | O1 | HOH | W | 21 | 31.636 | 40.357 | 28.787 | 1.00 | 62.00 | 8 | O |
| ATOM | 22 | O1 | HOH | W | 22 | 33.311 | 41.946 | 25.682 | 1.00 | 52.83 | 8 | O |
| ATOM | 23 | O1 | HOH | W | 23 | 30.668 | 37.676 | 29.602 | 1.00 | 63.49 | 8 | O |
| ATOM | 24 | O1 | HOH | W | 24 | 33.351 | 39.445 | 31.453 | 1.00 | 48.61 | 8 | O |
| ATOM | 25 | O1 | HOH | W | 25 | 10.588 | 51.177 | 19.875 | 1.00 | 46.32 | 8 | O |
| ATOM | 26 | O1 | HOH | W | 26 | 19.101 | 43.610 | 37.923 | 1.00 | 51.37 | 8 | O |
| ATOM | 27 | O1 | HOH | W | 27 | 32.756 | 48.007 | 12.395 | 1.00 | 51.00 | 8 | O |
| ATOM | 28 | O1 | HOH | W | 28 | 32.674 | 49.670 | 14.196 | 1.00 | 47.62 | 8 | O |
| ATOM | 29 | O1 | HOH | W | 29 | 25.585 | 41.165 | 19.391 | 1.00 | 52.32 | 8 | O |
| ATOM | 30 | O1 | HOH | W | 30 | 26.674 | 38.235 | 20.849 | 1.00 | 29.09 | 8 | O |
| ATOM | 31 | O1 | HOH | W | 31 | 26.207 | 39.346 | 19.156 | 1.00 | 59.19 | 8 | O |
| ATOM | 32 | O1 | HOH | W | 32 | 30.658 | 40.368 | 22.149 | 1.00 | 43.78 | 8 | O |
| ATOM | 33 | O1 | HOH | W | 33 | 39.583 | 39.129 | 6.901 | 1.00 | 35.61 | 8 | O |
| TER | | | | | | | | | | | | |
| ATOM | 1 | N | PHE | B | 0 | 12.996 | 16.960 | −2.638 | 1.00 | 62.36 | 7 | N |
| ATOM | 2 | CA | PHE | B | 0 | 13.546 | 17.179 | −1.290 | 1.00 | 63.39 | 6 | C |
| ATOM | 3 | C | PHE | B | 0 | 14.665 | 18.236 | −1.240 | 1.00 | 61.73 | 6 | C |
| ATOM | 4 | O | PHE | B | 0 | 15.236 | 18.590 | −2.265 | 1.00 | 63.41 | 8 | O |
| ATOM | 5 | CB | PHE | B | 0 | 14.061 | 15.949 | −0.554 | 1.00 | 64.35 | 6 | C |
| ATOM | 6 | CG | PHE | B | 0 | 15.522 | 15.648 | −0.749 | 1.00 | 63.64 | 6 | C |
| ATOM | 7 | CD1 | PHE | B | 0 | 16.338 | 15.502 | 0.355 | 1.00 | 64.10 | 6 | C |
| ATOM | 8 | CD2 | PHE | B | 0 | 16.018 | 15.319 | −2.002 | 1.00 | 62.56 | 6 | C |
| ATOM | 9 | CE1 | PHE | B | 0 | 17.700 | 15.317 | 0.177 | 1.00 | 62.88 | 6 | C |
| ATOM | 10 | CE2 | PHE | B | 0 | 17.331 | 14.900 | −2.130 | 1.00 | 62.77 | 6 | C |
| ATOM | 11 | CZ | PHE | B | 0 | 18.168 | 14.884 | −1.041 | 1.00 | 62.32 | 6 | C |
| ATOM | 12 | N | MET | B | 1 | 14.915 | 18.774 | −0.050 | 1.00 | 58.21 | 7 | N |
| ATOM | 13 | CA | MET | B | 1 | 15.923 | 19.807 | 0.060 | 1.00 | 53.82 | 6 | C |
| ATOM | 14 | C | MET | B | 1 | 16.955 | 19.497 | 1.120 | 1.00 | 49.19 | 6 | C |
| ATOM | 15 | O | MET | B | 1 | 16.766 | 18.589 | 1.894 | 1.00 | 50.18 | 8 | O |
| ATOM | 16 | CB | MET | B | 1 | 15.373 | 21.155 | 0.509 | 1.00 | 57.09 | 6 | C |
| ATOM | 17 | CG | MET | B | 1 | 14.751 | 21.937 | −0.600 | 1.00 | 63.61 | 6 | C |
| ATOM | 18 | SD | MET | B | 1 | 14.400 | 23.643 | −0.179 | 1.00 | 72.31 | 16 | S |
| ATOM | 19 | CE | MET | B | 1 | 15.756 | 24.598 | −0.919 | 1.00 | 69.90 | 6 | C |
| ATOM | 20 | N | PHE | B | 2 | 18.006 | 20.302 | 1.053 | 1.00 | 43.18 | 7 | N |
| ATOM | 21 | CA | PHE | B | 2 | 19.026 | 20.155 | 2.103 | 1.00 | 35.60 | 6 | C |
| ATOM | 22 | C | PHE | B | 2 | 18.763 | 21.471 | 2.803 | 1.00 | 34.55 | 6 | C |
| ATOM | 23 | O | PHE | B | 2 | 18.326 | 22.467 | 2.111 | 1.00 | 31.59 | 8 | O |
| ATOM | 24 | CB | PHE | B | 2 | 20.336 | 19.940 | 1.468 | 1.00 | 34.55 | 6 | C |
| ATOM | 25 | CG | PHE | B | 2 | 20.847 | 18.638 | 0.935 | 1.00 | 29.77 | 6 | C |
| ATOM | 26 | CD1 | PHE | B | 2 | 21.561 | 18.581 | −0.262 | 1.00 | 28.21 | 6 | C |
| ATOM | 27 | CD2 | PHE | B | 2 | 20.617 | 17.477 | 1.627 | 1.00 | 29.31 | 6 | C |
| ATOM | 28 | CE1 | PHE | B | 2 | 22.019 | 17.407 | −0.729 | 1.00 | 25.86 | 6 | C |
| ATOM | 29 | CE2 | PHE | B | 2 | 21.091 | 16.217 | 1.148 | 1.00 | 27.63 | 6 | C |
| ATOM | 30 | CZ | PHE | B | 2 | 21.789 | 16.218 | −0.031 | 1.00 | 26.07 | 6 | C |
| ATOM | 31 | N | LYS | B | 3 | 18.984 | 21.498 | 4.077 | 1.00 | 32.99 | 7 | N |
| ATOM | 32 | CA | LYS | B | 3 | 18.756 | 22.786 | 4.816 | 1.00 | 32.24 | 6 | C |
| ATOM | 33 | C | LYS | B | 3 | 20.081 | 23.176 | 5.453 | 1.00 | 33.54 | 6 | C |
| ATOM | 34 | O | LYS | B | 3 | 20.434 | 24.260 | 5.813 | 1.00 | 31.06 | 8 | O |
| ATOM | 35 | CB | LYS | B | 3 | 17.585 | 22.551 | 5.846 | 1.00 | 31.16 | 6 | C |
| ATOM | 36 | CG | LYS | B | 3 | 16.267 | 22.575 | 5.438 | 0.00 | 25.00 | 6 | C |
| ATOM | 37 | CD | LYS | B | 3 | 15.355 | 22.145 | 6.572 | 0.00 | 25.00 | 6 | C |
| ATOM | 38 | CE | LYS | B | 3 | 14.059 | 22.931 | 6.568 | 0.00 | 25.00 | 6 | C |
| ATOM | 39 | NZ | LYS | B | 3 | 13.024 | 22.289 | 7.424 | 0.00 | 25.00 | 7 | N |
| ATOM | 40 | N | HIS | B | 4 | 21.011 | 22.216 | 5.713 | 1.00 | 34.25 | 7 | N |
| ATOM | 41 | CA | HIS | B | 4 | 22.260 | 22.284 | 6.352 | 1.00 | 29.94 | 6 | C |
| ATOM | 42 | C | HIS | B | 4 | 23.478 | 21.656 | 5.713 | 1.00 | 30.78 | 6 | C |
| ATOM | 43 | O | HIS | B | 4 | 23.434 | 20.581 | 5.088 | 1.00 | 27.94 | 8 | O |
| ATOM | 44 | CB | HIS | B | 4 | 22.264 | 21.499 | 7.730 | 1.00 | 33.44 | 6 | C |
| ATOM | 45 | CG | HIS | B | 4 | 21.119 | 22.071 | 8.529 | 1.00 | 36.16 | 6 | C |
| ATOM | 46 | ND1 | HIS | B | 4 | 21.193 | 23.432 | 8.869 | 1.00 | 37.08 | 7 | N |
| ATOM | 47 | CD2 | HIS | B | 4 | 19.963 | 21.545 | 9.015 | 1.00 | 34.53 | 6 | C |
| ATOM | 48 | CE1 | HIS | B | 4 | 20.111 | 23.713 | 9.544 | 1.00 | 37.92 | 6 | C |
| ATOM | 49 | NE2 | HIS | B | 4 | 19.353 | 22.585 | 9.643 | 1.00 | 37.24 | 7 | N |
| ATOM | 50 | N | ILE | B | 5 | 24.590 | 22.333 | 6.051 | 1.00 | 30.46 | 7 | N |
| ATOM | 51 | CA | ILE | B | 5 | 25.930 | 21.943 | 5.677 | 1.00 | 30.39 | 6 | C |
| ATOM | 52 | C | ILE | B | 5 | 26.889 | 22.077 | 6.833 | 1.00 | 29.55 | 6 | C |
| ATOM | 53 | O | ILE | B | 5 | 26.819 | 23.051 | 7.619 | 1.00 | 30.51 | 8 | O |
| ATOM | 54 | CB | ILE | B | 5 | 26.349 | 22.790 | 4.436 | 1.00 | 32.00 | 6 | C |
| ATOM | 55 | CG1 | ILE | B | 5 | 25.864 | 21.889 | 3.272 | 1.00 | 33.25 | 6 | C |
| ATOM | 56 | CG2 | ILE | B | 5 | 27.841 | 22.990 | 4.361 | 1.00 | 31.19 | 6 | C |
| ATOM | 57 | CD1 | ILE | B | 5 | 25.790 | 22.533 | 1.974 | 1.00 | 35.69 | 6 | C |
| ATOM | 58 | N | ILE | B | 6 | 27.793 | 21.132 | 7.008 | 1.00 | 26.73 | 7 | N |
| ATOM | 59 | CA | ILE | B | 6 | 28.814 | 21.101 | 8.020 | 1.00 | 22.50 | 6 | C |
| ATOM | 60 | C | ILE | B | 6 | 30.118 | 21.068 | 7.263 | 1.00 | 21.02 | 6 | C |
| ATOM | 61 | O | ILE | B | 6 | 30.258 | 20.301 | 6.305 | 1.00 | 21.94 | 8 | O |
| ATOM | 62 | CB | ILE | B | 6 | 28.688 | 19.921 | 9.004 | 1.00 | 24.98 | 6 | C |
| ATOM | 63 | CG1 | ILE | B | 6 | 27.421 | 20.098 | 9.871 | 1.00 | 22.79 | 6 | C |
| ATOM | 64 | CG2 | ILE | B | 6 | 29.910 | 19.667 | 9.911 | 1.00 | 22.85 | 6 | C |
| ATOM | 65 | CD1 | ILE | B | 6 | 27.160 | 18.742 | 10.628 | 1.00 | 23.58 | 6 | C |
| ATOM | 66 | N | ALA | B | 7 | 31.106 | 21.888 | 7.569 | 1.00 | 18.11 | 7 | N |
| ATOM | 67 | CA | ALA | B | 7 | 32.379 | 21.825 | 6.823 | 1.00 | 15.72 | 6 | C |
| ATOM | 68 | C | ALA | B | 7 | 33.442 | 22.157 | 7.848 | 1.00 | 19.04 | 6 | C |
| ATOM | 69 | O | ALA | B | 7 | 32.987 | 22.511 | 8.965 | 1.00 | 18.48 | 8 | O |
| ATOM | 70 | CB | ALA | B | 7 | 32.304 | 22.763 | 5.676 | 1.00 | 19.74 | 6 | C |
| ATOM | 71 | N | ARG | B | 8 | 34.717 | 22.053 | 7.547 | 1.00 | 21.91 | 7 | N |
| ATOM | 72 | CA | ARG | B | 8 | 35.748 | 22.385 | 8.589 | 1.00 | 21.90 | 6 | C |
| ATOM | 73 | C | ARG | B | 8 | 36.903 | 23.048 | 7.916 | 1.00 | 20.32 | 6 | C |
| ATOM | 74 | O | ARG | B | 8 | 37.233 | 22.541 | 6.824 | 1.00 | 17.80 | 8 | O |
| ATOM | 75 | CB | ARG | B | 8 | 36.231 | 21.073 | 9.249 | 1.00 | 20.23 | 6 | C |
| ATOM | 76 | CG | ARG | B | 8 | 37.201 | 21.380 | 10.378 | 1.00 | 21.10 | 6 | C |
| ATOM | 77 | CD | ARG | B | 8 | 37.855 | 20.114 | 11.038 | 1.00 | 22.39 | 6 | C |
| ATOM | 78 | NE | ARG | B | 8 | 36.871 | 19.288 | 11.726 | 1.00 | 23.51 | 7 | N |
| ATOM | 79 | CZ | ARG | B | 8 | 36.424 | 19.663 | 12.928 | 1.00 | 24.23 | 6 | C |
| ATOM | 80 | NH1 | ARG | B | 8 | 36.850 | 20.781 | 13.528 | 1.00 | 27.58 | 7 | N |
| ATOM | 81 | NH2 | ARG | B | 8 | 35.560 | 18.915 | 13.510 | 1.00 | 22.42 | 7 | N |
| ATOM | 82 | N | THR | B | 9 | 37.497 | 24.103 | 8.447 | 1.00 | 21.98 | 7 | N |
| ATOM | 83 | CA | THR | B | 9 | 38.616 | 24.762 | 7.679 | 1.00 | 24.00 | 6 | C |
| ATOM | 84 | C | THR | B | 9 | 39.793 | 23.826 | 7.611 | 1.00 | 25.11 | 6 | C |
| ATOM | 85 | O | THR | B | 9 | 39.865 | 23.080 | 8.615 | 1.00 | 26.77 | 8 | O |
| ATOM | 86 | CB | THR | B | 9 | 38.941 | 25.983 | 8.592 | 1.00 | 24.67 | 6 | C |
| ATOM | 87 | OG1 | THR | B | 9 | 37.682 | 26.731 | 8.797 | 1.00 | 25.37 | 8 | O |
| ATOM | 88 | CG2 | THR | B | 9 | 39.916 | 26.905 | 7.973 | 1.00 | 23.69 | 6 | C |
| ATOM | 89 | N | PRO | B | 10 | 40.584 | 23.787 | 6.555 | 1.00 | 26.38 | 7 | N |
| ATOM | 90 | CA | PRO | B | 10 | 41.760 | 22.907 | 6.541 | 1.00 | 25.50 | 6 | C |
| ATOM | 91 | C | PRO | B | 10 | 42.784 | 23.351 | 7.535 | 1.00 | 29.11 | 6 | C |
| ATOM | 92 | O | PRO | B | 10 | 42.927 | 24.594 | 7.689 | 1.00 | 34.74 | 8 | O |
| ATOM | 93 | CB | PRO | B | 10 | 42.485 | 23.278 | 5.215 | 1.00 | 21.19 | 6 | C |
| ATOM | 94 | CG | PRO | B | 10 | 41.596 | 24.155 | 4.451 | 1.00 | 17.14 | 6 | C |
| ATOM | 95 | CD | PRO | B | 10 | 40.511 | 24.713 | 5.346 | 1.00 | 20.58 | 6 | C |
| ATOM | 96 | N | ALA | B | 11 | 43.580 | 22.501 | 8.131 | 1.00 | 32.94 | 7 | N |
| ATOM | 97 | CA | ALA | B | 11 | 44.707 | 22.780 | 9.025 | 1.00 | 31.80 | 6 | C |
| ATOM | 98 | C | ALA | B | 11 | 45.952 | 22.992 | 8.192 | 1.00 | 34.51 | 6 | C |
| ATOM | 99 | O | ALA | B | 11 | 46.080 | 22.628 | 7.021 | 1.00 | 31.91 | 8 | O |
| ATOM | 100 | CB | ALA | B | 11 | 45.135 | 21.478 | 9.751 | 1.00 | 34.92 | 6 | C |
| ATOM | 101 | N | ARG | B | 12 | 46.983 | 23.566 | 8.857 | 1.00 | 40.72 | 7 | N |
| ATOM | 102 | CA | ARG | B | 12 | 48.262 | 23.766 | 8.193 | 1.00 | 41.31 | 6 | C |
| ATOM | 103 | C | ARG | B | 12 | 48.844 | 22.358 | 7.966 | 1.00 | 40.01 | 6 | C |
| ATOM | 104 | O | ARG | B | 12 | 49.497 | 22.184 | 6.947 | 1.00 | 35.70 | 8 | O |
| ATOM | 105 | CB | ARG | B | 12 | 49.193 | 24.645 | 9.007 | 1.00 | 44.40 | 6 | C |
| ATOM | 106 | CG | ARG | B | 12 | 48.531 | 25.991 | 9.264 | 1.00 | 52.43 | 6 | C |
| ATOM | 107 | CD | ARG | B | 12 | 49.536 | 27.192 | 9.701 | 0.00 | 25.00 | 6 | C |
| ATOM | 108 | NE | ARG | B | 12 | 49.016 | 28.553 | 9.647 | 0.00 | 25.00 | 7 | N |
| ATOM | 109 | CZ | ARG | B | 12 | 49.478 | 29.496 | 8.838 | 0.00 | 25.00 | 6 | C |
| ATOM | 110 | NH1 | ARG | B | 12 | 50.491 | 29.233 | 8.027 | 0.00 | 25.00 | 7 | N |
| ATOM | 111 | NH2 | ARG | B | 12 | 48.922 | 30.699 | 8.841 | 0.00 | 25.00 | 7 | N |
| ATOM | 112 | N | SER | B | 13 | 48.529 | 21.397 | 8.841 | 1.00 | 38.57 | 7 | N |
| ATOM | 113 | CA | SER | B | 13 | 49.038 | 20.064 | 8.729 | 1.00 | 38.85 | 6 | C |
| ATOM | 114 | C | SER | B | 13 | 48.586 | 19.299 | 7.528 | 1.00 | 43.88 | 6 | C |
| ATOM | 115 | O | SER | B | 13 | 49.137 | 18.276 | 7.047 | 1.00 | 46.47 | 8 | O |
| ATOM | 116 | CB | SER | B | 13 | 48.895 | 19.331 | 10.015 | 1.00 | 39.71 | 6 | C |
| ATOM | 117 | OG | SER | B | 13 | 47.663 | 19.228 | 10.659 | 1.00 | 42.09 | 8 | O |
| ATOM | 118 | N | LEU | B | 14 | 47.600 | 19.789 | 6.820 | 1.00 | 46.15 | 7 | N |
| ATOM | 119 | CA | LEU | B | 14 | 47.105 | 19.313 | 5.570 | 1.00 | 50.77 | 6 | C |
| ATOM | 120 | C | LEU | B | 14 | 48.342 | 19.024 | 4.726 | 1.00 | 54.56 | 6 | C |
| ATOM | 121 | O | LEU | B | 14 | 48.478 | 17.976 | 4.128 | 1.00 | 57.78 | 8 | O |
| ATOM | 122 | CB | LEU | B | 14 | 46.510 | 20.467 | 4.729 | 1.00 | 52.16 | 6 | C |
| ATOM | 123 | CG | LEU | B | 14 | 45.170 | 20.293 | 4.055 | 1.00 | 53.42 | 6 | C |
| ATOM | 124 | CDI | LEU | B | 14 | 45.221 | 20.769 | 2.611 | 1.00 | 52.91 | 6 | C |
| ATOM | 125 | CD2 | LEU | B | 14 | 44.681 | 18.864 | 4.143 | 1.00 | 52.28 | 6 | C |
| ATOM | 126 | N | VAL | B | 15 | 49.191 | 20.032 | 4.575 | 1.00 | 59.93 | 7 | N |
| ATOM | 127 | CA | VAL | B | 15 | 50.410 | 19.890 | 3.827 | 1.00 | 66.21 | 6 | C |
| ATOM | 128 | C | VAL | B | 15 | 51.163 | 18.764 | 4.513 | 1.00 | 69.45 | 6 | C |
| ATOM | 129 | O | VAL | B | 15 | 51.792 | 19.169 | 5.485 | 1.00 | 68.94 | 8 | O |
| ATOM | 130 | CB | VAL | B | 15 | 51.391 | 21.080 | 3.867 | 1.00 | 67.73 | 6 | C |
| ATOM | 131 | CG1 | VAL | B | 15 | 52.751 | 20.619 | 3.325 | 1.00 | 69.54 | 6 | C |
| ATOM | 132 | CG2 | VAL | B | 15 | 50.799 | 22.209 | 3.039 | 1.00 | 66.01 | 6 | C |
| ATOM | 133 | N | ASP | B | 16 | 51.021 | 17.538 | 4.004 | 1.00 | 74.17 | 7 | N |
| ATOM | 134 | CA | ASP | B | 16 | 51.733 | 16.448 | 4.600 | 1.00 | 76.62 | 6 | C |
| ATOM | 135 | C | ASP | B | 16 | 51.165 | 15.040 | 4.436 | 1.00 | 79.32 | 6 | C |
| ATOM | 136 | O | ASP | B | 16 | 50.138 | 14.714 | 3.868 | 1.00 | 82.55 | 8 | O |
| ATOM | 137 | CB | ASP | B | 16 | 52.039 | 16.495 | 6.097 | 1.00 | 76.64 | 6 | C |
| ATOM | 138 | CG | ASP | B | 16 | 53.393 | 17.152 | 6.330 | 1.00 | 78.67 | 6 | C |
| ATOM | 139 | OD1 | ASP | B | 16 | 54.173 | 17.028 | 5.351 | 1.00 | 79.90 | 8 | O |
| ATOM | 140 | OD2 | ASP | B | 16 | 53.418 | 17.693 | 7.473 | 1.00 | 78.69 | 8 | O |
| ATOM | 141 | N | GLY | B | 17 | 52.029 | 14.254 | 5.071 | 1.00 | 78.62 | 7 | N |
| ATOM | 142 | CA | GLY | B | 17 | 52.032 | 12.812 | 5.134 | 1.00 | 78.49 | 6 | C |
| ATOM | 143 | C | GLY | B | 17 | 53.511 | 12.667 | 4.673 | 1.00 | 78.29 | 6 | C |
| ATOM | 144 | O | GLY | B | 17 | 54.044 | 13.544 | 3.973 | 1.00 | 78.69 | 8 | O |
| ATOM | 145 | N | LEU | B | 18 | 54.110 | 11.545 | 5.065 | 1.00 | 76.76 | 7 | N |
| ATOM | 146 | CA | LEU | B | 18 | 55.507 | 11.329 | 4.708 | 1.00 | 73.67 | 6 | C |
| ATOM | 147 | C | LEU | B | 18 | 55.650 | 10.934 | 3.263 | 1.00 | 73.42 | 6 | C |
| ATOM | 148 | O | LEU | B | 18 | 56.734 | 10.884 | 2.676 | 1.00 | 75.40 | 8 | O |
| ATOM | 149 | CB | LEU | B | 18 | 56.036 | 10.166 | 5.551 | 1.00 | 71.65 | 6 | C |
| ATOM | 150 | CG | LEU | B | 18 | 56.362 | 10.550 | 6.997 | 1.00 | 70.44 | 6 | C |
| ATOM | 151 | CD1 | LEU | B | 18 | 55.219 | 11.267 | 7.696 | 1.00 | 70.33 | 6 | C |
| ATOM | 152 | CD2 | LEU | B | 18 | 56.764 | 9.332 | 7.808 | 1.00 | 68.67 | 6 | C |
| ATOM | 153 | N | THR | B | 19 | 54.497 | 10.557 | 2.741 | 1.00 | 72.30 | 7 | N |
| ATOM | 154 | CA | THR | B | 19 | 54.556 | 10.075 | 1.357 | 1.00 | 74.10 | 6 | C |
| ATOM | 155 | C | THR | B | 19 | 53.556 | 10.855 | 0.516 | 1.00 | 75.60 | 6 | C |
| ATOM | 156 | O | THR | B | 19 | 52.456 | 11.174 | 0.965 | 1.00 | 78.14 | 8 | O |
| ATOM | 157 | CB | THR | B | 19 | 54.178 | 8.596 | 1.449 | 1.00 | 71.63 | 6 | C |
| ATOM | 158 | OG1 | THR | B | 19 | 52.866 | 8.714 | 2.070 | 1.00 | 71.21 | 8 | O |
| ATOM | 159 | CG2 | THR | B | 19 | 55.122 | 7.698 | 2.228 | 1.00 | 70.76 | 6 | C |
| ATOM | 160 | N | SER | B | 20 | 53.992 | 11.144 | −0.700 | 1.00 | 75.63 | 7 | N |
| ATOM | 161 | CA | SER | B | 20 | 53.162 | 11.897 | −1.622 | 1.00 | 77.65 | 6 | C |
| ATOM | 162 | C | SER | B | 20 | 52.872 | 11.134 | −2.925 | 1.00 | 77.69 | 6 | C |
| ATOM | 163 | O | SER | B | 20 | 53.566 | 10.189 | −3.295 | 1.00 | 78.40 | 8 | O |
| ATOM | 164 | CB | SER | B | 20 | 53.783 | 13.263 | −1.965 | 1.00 | 78.23 | 6 | C |
| ATOM | 165 | OG | SER | B | 20 | 55.140 | 12.783 | −2.575 | 0.00 | 25.00 | 8 | O |
| ATOM | 166 | N | SER | B | 21 | 51.792 | 11.575 | −3.582 | 1.00 | 76.63 | 7 | N |
| ATOM | 167 | CA | SER | B | 21 | 51.326 | 10.965 | −4.826 | 1.00 | 75.04 | 6 | C |
| ATOM | 168 | C | SER | B | 21 | 51.763 | 11.858 | −5.952 | 1.00 | 72.68 | 6 | C |
| ATOM | 169 | O | SER | B | 21 | 51.551 | 13.068 | −5.870 | 1.00 | 77.30 | 8 | O |
| ATOM | 170 | CB | SER | B | 21 | 49.798 | 10.820 | −4.858 | 1.00 | 74.64 | 6 | C |
| ATOM | 171 | OG | SER | B | 21 | 49.298 | 12.134 | −4.628 | 1.00 | 77.06 | 8 | O |
| ATOM | 172 | N | HIS | B | 22 | 52.344 | 11.434 | −7.018 | 1.00 | 67.97 | 7 | N |
| ATOM | 173 | CA | HIS | B | 22 | 52.785 | 12.194 | −8.135 | 1.00 | 64.63 | 6 | C |
| ATOM | 174 | C | HIS | B | 22 | 52.143 | 13.513 | −8.403 | 1.00 | 63.58 | 6 | C |
| ATOM | 175 | O | HIS | B | 22 | 52.748 | 14.263 | −9.191 | 1.00 | 67.38 | 8 | O |
| ATOM | 176 | CB | HIS | B | 22 | 52.658 | 11.304 | −9.387 | 1.00 | 65.37 | 6 | C |
| ATOM | 177 | CG | HIS | B | 22 | 53.616 | 10.134 | −9.252 | 0.00 | 25.00 | 6 | C |
| ATOM | 178 | ND1 | HIS | B | 22 | 54.989 | 10.121 | −9.110 | 0.00 | 25.00 | 6 | N |
| ATOM | 179 | CD2 | HIS | B | 22 | 53.220 | 8.843 | −9.221 | 0.00 | 25.00 | 6 | C |
| ATOM | 180 | CE1 | HIS | B | 22 | 55.403 | 8.870 | −9.000 | 0.00 | 25.00 | 6 | C |
| ATOM | 181 | NE2 | HIS | B | 22 | 54.349 | 8.075 | −9.067 | 0.00 | 25.00 | 7 | N |
| ATOM | 182 | N | LEU | B | 23 | 51.025 | 13.942 | −7.883 | 1.00 | 62.50 | 7 | N |
| ATOM | 183 | CA | LEU | B | 23 | 50.398 | 15.232 | −8.039 | 1.00 | 63.22 | 6 | C |
| ATOM | 184 | C | LEU | B | 23 | 51.204 | 16.276 | −7.253 | 1.00 | 62.01 | 6 | C |
| ATOM | 185 | O | LEU | B | 23 | 51.218 | 17.448 | −7.531 | 1.00 | 61.98 | 8 | O |
| ATOM | 186 | CB | LEU | B | 23 | 48.990 | 15.184 | −7.485 | 1.00 | 62.42 | 6 | C |
| ATOM | 187 | CG | LEU | B | 23 | 48.038 | 14.287 | −8.314 | 1.00 | 63.66 | 6 | C |
| ATOM | 188 | CD1 | LEU | B | 23 | 46.699 | 14.254 | −7.594 | 1.00 | 64.12 | 6 | C |
| ATOM | 189 | CD2 | LEU | B | 23 | 47.905 | 14.806 | −9.737 | 1.00 | 64.69 | 6 | C |
| ATOM | 190 | N | GLY | B | 24 | 51.904 | 15.749 | −6.256 | 1.00 | 61.33 | 7 | N |
| ATOM | 191 | CA | GLY | B | 24 | 52.789 | 16.532 | −5.458 | 1.00 | 61.50 | 6 | C |
| ATOM | 192 | C | GLY | B | 24 | 51.836 | 17.037 | −4.385 | 1.00 | 62.42 | 6 | C |
| ATOM | 193 | O | GLY | B | 24 | 50.654 | 16.747 | −4.448 | 1.00 | 64.98 | 8 | O |
| ATOM | 194 | N | LYS | B | 25 | 52.448 | 17.821 | −3.554 | 1.00 | 62.18 | 7 | N |
| ATOM | 195 | CA | LYS | B | 25 | 51.823 | 18.407 | −2.433 | 1.00 | 60.21 | 6 | C |
| ATOM | 196 | C | LYS | B | 25 | 51.063 | 19.725 | −2.485 | 1.00 | 54.10 | 6 | C |
| ATOM | 197 | O | LYS | B | 25 | 51.279 | 20.654 | −3.223 | 1.00 | 53.62 | 8 | O |
| ATOM | 198 | CB | LYS | B | 25 | 52.834 | 18.656 | −1.277 | 1.00 | 65.47 | 6 | C |
| ATOM | 199 | CG | LYS | B | 25 | 53.559 | 17.365 | −0.966 | 1.00 | 72.86 | 6 | C |
| ATOM | 200 | CD | LYS | B | 25 | 54.556 | 17.496 | 0.178 | 1.00 | 77.18 | 6 | C |
| ATOM | 201 | CE | LYS | B | 25 | 55.275 | 16.178 | 0.400 | 1.00 | 83.60 | 6 | C |
| ATOM | 202 | NZ | LYS | B | 25 | 56.219 | 16.116 | 1.558 | 1.00 | 86.25 | 7 | N |
| ATOM | 203 | N | PRO | B | 26 | 50.103 | 19.717 | −1.544 | 1.00 | 46.97 | 7 | N |
| ATOM | 204 | CA | PRO | B | 26 | 49.186 | 20.786 | −1.325 | 1.00 | 43.27 | 6 | C |
| ATOM | 205 | C | PRO | B | 26 | 49.763 | 22.103 | −0.792 | 1.00 | 38.14 | 6 | C |
| ATOM | 206 | O | PRO | B | 26 | 50.372 | 21.965 | 0.262 | 1.00 | 35.26 | 8 | O |
| ATOM | 207 | CB | PRO | B | 26 | 48.324 | 20.296 | −0.126 | 1.00 | 42.06 | 6 | C |
| ATOM | 208 | CG | PRO | B | 26 | 48.486 | 18.830 | −0.109 | 1.00 | 44.29 | 6 | C |
| ATOM | 209 | CD | PRO | B | 26 | 49.857 | 18.556 | −0.637 | 1.00 | 45.04 | 6 | C |
| ATOM | 210 | N | ASP | B | 27 | 49.564 | 23.172 | −1.497 | 1.00 | 34.44 | 7 | N |
| ATOM | 211 | CA | ASP | B | 27 | 50.018 | 24.454 | −0.983 | 1.00 | 36.61 | 6 | C |
| ATOM | 212 | C | ASP | B | 27 | 48.934 | 24.848 | 0.014 | 1.00 | 38.42 | 6 | C |
| ATOM | 213 | O | ASP | B | 27 | 47.732 | 25.096 | −0.314 | 1.00 | 40.93 | 8 | O |
| ATOM | 214 | CB | ASP | B | 27 | 50.079 | 25.392 | −2.145 | 1.00 | 37.37 | 6 | C |
| ATOM | 215 | CG | ASP | B | 27 | 50.062 | 26.902 | −1.988 | 1.00 | 41.23 | 6 | C |
| ATOM | 216 | OD1 | ASP | B | 27 | 50.216 | 27.362 | −0.819 | 1.00 | 37.92 | 8 | O |
| ATOM | 217 | OD2 | ASP | B | 27 | 49.893 | 27.585 | −3.085 | 1.00 | 40.68 | 8 | O |
| ATOM | 218 | N | TYR | B | 28 | 49.194 | 24.977 | 1.279 | 1.00 | 38.86 | 7 | N |
| ATOM | 219 | CA | TYR | B | 28 | 48.244 | 25.378 | 2.270 | 1.00 | 39.16 | 6 | C |
| ATOM | 220 | C | TYR | B | 28 | 47.542 | 26.646 | 1.804 | 1.00 | 39.02 | 6 | C |
| ATOM | 221 | O | TYR | B | 28 | 46.322 | 26.722 | 1.763 | 1.00 | 40.07 | 8 | O |
| ATOM | 222 | CB | TYR | B | 28 | 48.952 | 25.717 | 3.577 | 1.00 | 38.50 | 6 | C |
| ATOM | 223 | CG | TYR | B | 28 | 48.012 | 26.120 | 4.655 | 1.00 | 42.85 | 6 | C |
| ATOM | 224 | CD1 | TYR | B | 28 | 46.929 | 25.286 | 4.953 | 1.00 | 43.17 | 6 | C |
| ATOM | 225 | CD2 | TYR | B | 28 | 48.158 | 27.299 | 5.394 | 1.00 | 44.23 | 6 | C |
| ATOM | 226 | CE1 | TYR | B | 28 | 46.059 | 25.667 | 5.963 | 1.00 | 45.91 | 6 | C |
| ATOM | 227 | CE2 | TYR | B | 28 | 47.264 | 27.639 | 6.396 | 1.00 | 45.36 | 6 | C |
| ATOM | 228 | CZ | TYR | B | 28 | 46.212 | 26.819 | 6.679 | 1.00 | 45.35 | 6 | C |
| ATOM | 229 | OH | TYR | B | 28 | 45.301 | 27.134 | 7.672 | 1.00 | 51.11 | 8 | O |
| ATOM | 230 | N | ALA | B | 29 | 48.233 | 27.695 | 1.451 | 1.00 | 38.57 | 7 | N |
| ATOM | 231 | CA | ALA | B | 29 | 47.634 | 28.955 | 1.071 | 1.00 | 36.27 | 6 | C |
| ATOM | 232 | C | ALA | B | 29 | 46.694 | 28.710 | −0.055 | 1.00 | 36.45 | 6 | C |
| ATOM | 233 | O | ALA | B | 29 | 45.593 | 29.222 | 0.086 | 1.00 | 40.46 | 8 | O |
| ATOM | 234 | CB | ALA | B | 29 | 48.545 | 30.130 | 0.750 | 1.00 | 32.06 | 6 | C |
| ATOM | 235 | N | LYS | B | 30 | 46.962 | 28.011 | −1.124 | 1.00 | 37.09 | 7 | N |
| ATOM | 236 | CA | LYS | B | 30 | 45.904 | 27.930 | −2.188 | 1.00 | 34.84 | 6 | C |
| ATOM | 237 | C | LYS | B | 30 | 44.746 | 27.100 | −1.644 | 1.00 | 35.26 | 6 | C |
| ATOM | 238 | O | LYS | B | 30 | 43.579 | 27.286 | −1.872 | 1.00 | 34.93 | 8 | O |
| ATOM | 239 | CB | LYS | B | 30 | 46.573 | 27.382 | −3.398 | 1.00 | 32.95 | 6 | C |
| ATOM | 240 | CG | LYS | B | 30 | 45.874 | 27.490 | −4.712 | 1.00 | 37.29 | 6 | C |
| ATOM | 241 | CD | LYS | B | 30 | 46.565 | 26.521 | −5.713 | 1.00 | 35.24 | 6 | C |
| ATOM | 242 | CE | LYS | B | 30 | 46.445 | 26.917 | −7.069 | 0.00 | 25.00 | 6 | C |
| ATOM | 243 | NZ | LYS | B | 30 | 46.975 | 25.760 | −7.896 | 0.00 | 25.00 | 7 | N |
| ATOM | 244 | N | ALA | B | 31 | 45.048 | 26.085 | −0.821 | 1.00 | 32.73 | 7 | N |
| ATOM | 245 | CA | ALA | B | 31 | 44.060 | 25.210 | −0.244 | 1.00 | 32.49 | 6 | C |
| ATOM | 246 | C | ALA | B | 31 | 43.086 | 26.066 | 0.567 | 1.00 | 32.89 | 6 | C |
| ATOM | 247 | O | ALA | B | 31 | 41.854 | 25.835 | 0.508 | 1.00 | 31.92 | 8 | O |
| ATOM | 248 | CB | ALA | B | 31 | 44.597 | 24.082 | 0.649 | 1.00 | 26.79 | 6 | C |
| ATOM | 249 | N | LEU | B | 32 | 43.685 | 26.998 | 1.311 | 1.00 | 34.13 | 7 | N |
| ATOM | 250 | CA | LEU | B | 32 | 42.811 | 27.865 | 2.169 | 1.00 | 34.01 | 6 | C |
| ATOM | 251 | C | LEU | B | 32 | 41.851 | 28.640 | 1.281 | 1.00 | 30.20 | 6 | C |
| ATOM | 252 | O | LEU | B | 32 | 40.688 | 28.816 | 1.592 | 1.00 | 29.48 | 8 | O |
| ATOM | 253 | CB | LEU | B | 32 | 43.662 | 28.839 | 2.992 | 1.00 | 34.11 | 6 | C |
| ATOM | 254 | CG | LEU | B | 32 | 43.041 | 29.042 | 4.386 | 1.00 | 34.32 | 6 | C |
| ATOM | 255 | CD1 | LEU | B | 32 | 43.313 | 27.811 | 5.186 | 1.00 | 33.82 | 6 | C |
| ATOM | 256 | CD2 | LEU | B | 32 | 43.532 | 30.261 | 5.094 | 1.00 | 33.98 | 6 | C |
| ATOM | 257 | N | GLU | B | 33 | 42.238 | 29.149 | 0.143 | 1.00 | 28.05 | 7 | N |
| ATOM | 258 | CA | GLU | B | 33 | 41.452 | 29.884 | −0.783 | 1.00 | 26.37 | 6 | C |
| ATOM | 259 | C | GLU | B | 33 | 40.455 | 28.979 | −1.519 | 1.00 | 27.62 | 6 | C |
| ATOM | 260 | O | GLU | B | 33 | 39.300 | 29.369 | −1.820 | 1.00 | 26.94 | 8 | O |
| ATOM | 261 | CB | GLU | B | 33 | 42.378 | 30.567 | −1.748 | 1.00 | 28.61 | 6 | C |
| ATOM | 262 | CG | GLU | B | 33 | 43.210 | 31.691 | −1.285 | 1.00 | 34.19 | 6 | C |
| ATOM | 263 | CD | GLU | B | 33 | 42.577 | 32.829 | −0.527 | 1.00 | 38.16 | 6 | C |
| ATOM | 264 | OE1 | GLU | B | 33 | 41.719 | 33.594 | −1.006 | 1.00 | 40.69 | 8 | O |
| ATOM | 265 | OE2 | GLU | B | 33 | 42.878 | 33.093 | 0.659 | 1.00 | 39.45 | 8 | O |
| ATOM | 266 | N | GLN | B | 34 | 40.859 | 27.698 | −1.817 | 1.00 | 24.90 | 7 | N |
| ATOM | 267 | CA | GLN | B | 34 | 39.942 | 26.838 | −2.507 | 1.00 | 24.09 | 6 | C |
| ATOM | 268 | C | GLN | B | 34 | 38.761 | 26.567 | −1.588 | 1.00 | 23.49 | 6 | C |
| ATOM | 269 | O | GLN | B | 34 | 37.572 | 26.649 | −1.838 | 1.00 | 21.28 | 8 | O |
| ATOM | 270 | CB | GLN | B | 34 | 40.662 | 25.653 | −3.128 | 1.00 | 27.12 | 6 | C |
| ATOM | 271 | CG | GLN | B | 34 | 41.704 | 26.043 | −4.134 | 1.00 | 29.89 | 6 | C |
| ATOM | 272 | CD | GLN | B | 34 | 42.307 | 24.869 | −4.861 | 1.00 | 31.87 | 6 | C |
| ATOM | 273 | OE1 | GLN | B | 34 | 42.407 | 23.832 | −4.173 | 1.00 | 34.54 | 8 | O |
| ATOM | 274 | NE2 | GLN | B | 34 | 42.639 | 25.096 | −6.113 | 1.00 | 28.43 | 7 | N |
| ATOM | 275 | N | HIS | B | 35 | 39.062 | 26.304 | −0.303 | 1.00 | 26.75 | 7 | N |
| ATOM | 276 | CA | HIS | B | 35 | 38.088 | 26.088 | 0.714 | 1.00 | 23.52 | 6 | C |
| ATOM | 277 | C | HIS | B | 35 | 37.131 | 27.284 | 0.851 | 1.00 | 25.05 | 6 | C |
| ATOM | 278 | O | HIS | B | 35 | 35.909 | 27.010 | 1.036 | 1.00 | 26.89 | 8 | O |
| ATOM | 279 | CB | HIS | B | 35 | 38.684 | 25.698 | 2.063 | 1.00 | 18.08 | 6 | C |
| ATOM | 280 | CG | HIS | B | 35 | 37.626 | 25.072 | 2.982 | 1.00 | 17.66 | 6 | C |
| ATOM | 281 | ND1 | HIS | B | 35 | 37.268 | 23.719 | 2.888 | 1.00 | 18.75 | 7 | N |
| ATOM | 282 | CD2 | HIS | B | 35 | 36.868 | 25.546 | 3.976 | 1.00 | 15.01 | 6 | C |
| ATOM | 283 | CE1 | HIS | B | 35 | 36.318 | 23.419 | 3.824 | 1.00 | 13.30 | 6 | C |
| ATOM | 284 | NE2 | HIS | B | 35 | 36.060 | 24.517 | 4.493 | 1.00 | 15.39 | 7 | N |
| ATOM | 285 | N | ASN | B | 36 | 37.635 | 28.486 | 0.885 | 1.00 | 21.09 | 7 | N |
| ATOM | 286 | CA | ASN | B | 36 | 36.751 | 29.628 | 1.076 | 1.00 | 23.45 | 6 | C |
| ATOM | 287 | C | ASN | B | 36 | 35.867 | 29.773 | −0.116 | 1.00 | 24.18 | 6 | C |
| ATOM | 288 | O | ASN | B | 36 | 34.664 | 30.039 | −0.013 | 1.00 | 24.11 | 8 | O |
| ATOM | 289 | CB | ASN | B | 36 | 37.499 | 30.938 | 1.353 | 1.00 | 27.50 | 6 | C |
| ATOM | 290 | CG | ASN | B | 36 | 36.683 | 32.178 | 1.309 | 1.00 | 29.07 | 6 | C |
| ATOM | 291 | OD1 | ASN | B | 36 | 36.009 | 32.472 | 2.323 | 1.00 | 34.53 | 8 | O |
| ATOM | 292 | ND2 | ASN | B | 36 | 36.595 | 33.011 | 0.283 | 1.00 | 27.04 | 7 | N |
| ATOM | 293 | N | ALA | B | 37 | 36.316 | 29.537 | −1.316 | 1.00 | 27.18 | 7 | N |
| ATOM | 294 | CA | ALA | B | 37 | 35.568 | 29.585 | −2.571 | 1.00 | 27.32 | 6 | C |
| ATOM | 295 | C | ALA | B | 37 | 34.518 | 28.493 | −2.530 | 1.00 | 26.59 | 6 | C |
| ATOM | 296 | O | ALA | B | 37 | 33.395 | 28.688 | −3.018 | 1.00 | 30.22 | 8 | O |
| ATOM | 297 | CB | ALA | B | 37 | 36.388 | 29.455 | −3.825 | 1.00 | 20.13 | 6 | C |
| ATOM | 298 | N | TYR | B | 38 | 34.852 | 27.445 | −1.801 | 1.00 | 25.56 | 7 | N |
| ATOM | 299 | CA | TYR | B | 38 | 33.871 | 26.313 | −1.679 | 1.00 | 23.33 | 6 | C |
| ATOM | 300 | C | TYR | B | 38 | 32.704 | 26.760 | −0.825 | 1.00 | 24.73 | 6 | C |
| ATOM | 301 | O | TYR | B | 38 | 31.548 | 26.437 | −1.118 | 1.00 | 23.97 | 8 | O |
| ATOM | 302 | CB | TYR | B | 38 | 34.571 | 25.103 | −1.141 | 1.00 | 17.31 | 6 | C |
| ATOM | 303 | CG | TYR | B | 38 | 33.792 | 23.847 | −0.847 | 1.00 | 21.19 | 6 | C |
| ATOM | 304 | CD1 | TYR | B | 38 | 33.289 | 23.035 | −1.855 | 1.00 | 21.93 | 6 | C |
| ATOM | 305 | CD2 | TYR | B | 38 | 33.527 | 23.427 | 0.458 | 1.00 | 19.92 | 6 | C |
| ATOM | 306 | CE1 | TYR | B | 38 | 32.581 | 21.896 | −1.573 | 1.00 | 22.41 | 6 | C |
| ATOM | 307 | CE2 | TYR | B | 38 | 32.815 | 22.278 | 0.727 | 1.00 | 21.98 | 6 | C |
| ATOM | 308 | CZ | TYR | B | 38 | 32.335 | 21.498 | −0.290 | 1.00 | 22.40 | 6 | C |
| ATOM | 309 | OH | TYR | B | 38 | 31.592 | 20.292 | −0.069 | 1.00 | 21.43 | 8 | O |
| ATOM | 310 | N | ILE | B | 39 | 33.050 | 27.397 | 0.304 | 1.00 | 24.72 | 7 | N |
| ATOM | 311 | CA | ILE | B | 39 | 32.138 | 27.925 | 1.286 | 1.00 | 23.52 | 6 | C |
| ATOM | 312 | C | ILE | B | 39 | 31.266 | 28.981 | 0.595 | 1.00 | 26.65 | 6 | C |
| ATOM | 313 | O | ILE | B | 39 | 30.026 | 28.856 | 0.735 | 1.00 | 27.47 | 8 | O |
| ATOM | 314 | CB | ILE | B | 39 | 32.922 | 28.347 | 2.532 | 1.00 | 20.42 | 6 | C |
| ATOM | 315 | CG1 | ILE | B | 39 | 33.613 | 27.228 | 3.331 | 1.00 | 17.66 | 6 | C |
| ATOM | 316 | CG2 | ILE | B | 39 | 31.769 | 28.908 | 3.423 | 1.00 | 23.85 | 6 | C |
| ATOM | 317 | CD1 | ILE | B | 39 | 32.595 | 26.011 | 3.566 | 1.00 | 16.59 | 6 | C |
| ATOM | 318 | N | ARG | B | 40 | 31.778 | 29.934 | −0.174 | 1.00 | 26.25 | 7 | N |
| ATOM | 319 | CA | ARG | B | 40 | 31.010 | 30.931 | −0.911 | 1.00 | 24.76 | 6 | C |
| ATOM | 320 | C | ARG | B | 40 | 30.089 | 30.285 | −1.923 | 1.00 | 28.77 | 6 | C |
| ATOM | 321 | O | ARG | B | 40 | 28.980 | 30.846 | −2.107 | 1.00 | 34.37 | 8 | O |
| ATOM | 322 | CB | ARG | B | 40 | 31.855 | 32.024 | −1.708 | 1.00 | 17.72 | 6 | C |
| ATOM | 323 | CG | ARG | B | 40 | 32.617 | 32.667 | −0.480 | 1.00 | 21.82 | 6 | C |
| ATOM | 324 | CD | ARG | B | 40 | 33.629 | 33.686 | −0.930 | 1.00 | 23.74 | 6 | C |
| ATOM | 325 | NE | ARG | B | 40 | 32.934 | 34.900 | −1.321 | 1.00 | 31.35 | 7 | N |
| ATOM | 326 | CZ | ARG | B | 40 | 33.182 | 36.098 | −1.810 | 1.00 | 34.06 | 6 | C |
| ATOM | 327 | NH1 | ARG | B | 40 | 34.423 | 36.461 | −2.106 | 1.00 | 39.39 | 7 | N |
| ATOM | 328 | NH2 | ARG | B | 40 | 32.257 | 37.035 | −2.057 | 1.00 | 33.60 | 7 | N |
| ATOM | 329 | N | ALA | B | 41 | 30.350 | 29.200 | −2.584 | 1.00 | 26.58 | 7 | N |
| ATOM | 330 | CA | ALA | B | 41 | 29.392 | 28.617 | −3.528 | 1.00 | 24.86 | 6 | C |
| ATOM | 331 | C | ALA | B | 41 | 28.308 | 27.984 | −2.681 | 1.00 | 27.27 | 6 | C |
| ATOM | 332 | O | ALA | B | 41 | 27.114 | 28.088 | −2.981 | 1.00 | 32.34 | 8 | O |
| ATOM | 333 | CB | ALA | B | 41 | 30.071 | 27.474 | −4.325 | 1.092 | 5.32 | 6 | C |
| ATOM | 334 | N | LEU | B | 42 | 28.656 | 27.274 | −1.582 | 1.00 | 25.24 | 7 | N |
| ATOM | 335 | CA | LEU | B | 42 | 27.567 | 26.670 | −0.810 | 1.00 | 26.25 | 6 | C |
| ATOM | 336 | C | LEU | B | 42 | 26.752 | 27.807 | −0.191 | 1.00 | 29.52 | 6 | C |
| ATOM | 337 | O | LEU | B | 42 | 25.579 | 27.590 | −0.033 | 1.00 | 32.32 | 8 | O |
| ATOM | 338 | CB | LEU | B | 42 | 27.991 | 25.726 | 0.273 | 1.00 | 23.81 | 6 | C |
| ATOM | 339 | CG | LEU | B | 42 | 28.574 | 24.392 | −0.164 | 1.00 | 22.39 | 6 | C |
| ATOM | 340 | CD1 | LEU | B | 42 | 29.548 | 23.891 | 0.944 | 1.00 | 14.74 | 6 | C |
| ATOM | 341 | CD2 | LEU | B | 42 | 27.467 | 23.397 | −0.406 | 1.00 | 20.59 | 6 | C |
| ATOM | 342 | N | GLN | B | 43 | 27.182 | 29.013 | 0.148 | 1.00 | 31.16 | 7 | N |
| ATOM | 343 | CA | GLN | B | 43 | 26.397 | 30.118 | 0.645 | 1.00 | 31.34 | 6 | C |
| ATOM | 344 | C | GLN | B | 43 | 25.320 | 30.551 | −0.342 | 1.00 | 33.64 | 6 | C |
| ATOM | 345 | O | GLN | B | 43 | 24.364 | 31.268 | −0.020 | 1.00 | 36.49 | 8 | O |
| ATOM | 346 | CB | GLN | B | 43 | 27.225 | 31.400 | 0.841 | 1.00 | 26.40 | 6 | C |
| ATOM | 347 | CG | GLN | B | 43 | 27.987 | 31.421 | 2.105 | 1.00 | 29.57 | 6 | C |
| ATOM | 348 | CD | GLN | B | 43 | 28.908 | 32.609 | 2.299 | 1.00 | 29.10 | 6 | C |
| ATOM | 349 | OE1 | GLN | B | 43 | 28.535 | 33.674 | 2.719 | 1.00 | 33.22 | 8 | O |
| ATOM | 350 | NE2 | GLN | B | 43 | 30.150 | 32.508 | 2.014 | 1.00 | 29.22 | 7 | N |
| ATOM | 351 | N | THR | B | 44 | 25.398 | 30.207 | −1.577 | 1.00 | 34.28 | 7 | N |
| ATOM | 352 | CA | THR | B | 44 | 24.486 | 30.460 | −2.685 | 1.00 | 36.60 | 6 | C |
| ATOM | 353 | C | THR | B | 44 | 23.465 | 29.345 | −2.765 | 1.00 | 36.65 | 6 | C |
| ATOM | 354 | O | THR | B | 44 | 22.462 | 29.524 | −3.454 | 1.00 | 39.15 | 8 | O |
| ATOM | 355 | CB | THR | B | 44 | 25.459 | 30.490 | −3.907 | 1.00 | 39.04 | 6 | C |
| ATOM | 356 | OG1 | THR | B | 44 | 26.081 | 31.812 | −4.038 | 1.00 | 38.48 | 8 | O |
| ATOM | 357 | CG2 | THR | B | 44 | 24.846 | 29.983 | −5.142 | 1.00 | 38.73 | 6 | C |
| ATOM | 358 | N | CYS | B | 45 | 23.580 | 28.219 | −2.032 | 1.00 | 32.93 | 7 | N |
| ATOM | 359 | CA | CYS | B | 45 | 22.606 | 27.148 | −2.151 | 1.00 | 33.58 | 6 | C |
| ATOM | 360 | C | CYS | B | 45 | 21.429 | 27.050 | −1.205 | 1.00 | 36.10 | 6 | C |
| ATOM | 361 | O | CYS | B | 45 | 20.842 | 25.950 | −1.078 | 1.00 | 37.61 | 8 | O |
| ATOM | 362 | CB | CYS | B | 45 | 23.361 | 25.802 | −2.130 | 1.00 | 29.46 | 6 | C |
| ATOM | 363 | SG | CYS | B | 45 | 24.625 | 25.839 | −3.456 | 1.00 | 27.64 | 16 | S |
| ATOM | 364 | N | ASP | B | 46 | 20.942 | 28.115 | −0.564 | 1.00 | 34.51 | 7 | N |
| ATOM | 365 | CA | ASP | B | 46 | 19.812 | 28.030 | 0.318 | 1.00 | 34.93 | 6 | C |
| ATOM | 366 | C | ASP | B | 46 | 19.946 | 26.997 | 1.411 | 1.00 | 33.58 | 6 | C |
| ATOM | 367 | O | ASP | B | 46 | 19.068 | 26.137 | 1.668 | 1.00 | 30.36 | 8 | O |
| ATOM | 368 | CB | ASP | B | 46 | 18.490 | 27.782 | −0.412 | 1.00 | 41.76 | 6 | C |
| ATOM | 369 | CG | ASP | B | 46 | 18.131 | 28.914 | −1.336 | 1.00 | 52.48 | 6 | C |
| ATOM | 370 | OD1 | ASP | B | 46 | 17.622 | 30.020 | −0.955 | 1.00 | 57.16 | 8 | O |
| ATOM | 371 | OD2 | ASP | B | 46 | 18.323 | 28.834 | −2.590 | 1.00 | 57.97 | 8 | O |
| ATOM | 372 | N | VAL | B | 47 | 21.058 | 27.087 | 2.117 | 1.00 | 32.40 | 7 | N |
| ATOM | 373 | CA | VAL | B | 47 | 21.353 | 26.182 | 3.229 | 1.00 | 29.14 | 6 | C |
| ATOM | 374 | C | VAL | B | 47 | 22.062 | 26.936 | 4.292 | 1.00 | 27.97 | 6 | G |
| ATOM | 375 | O | VAL | B | 47 | 22.712 | 27.944 | 3.936 | 1.00 | 30.56 | 8 | O |
| ATOM | 376 | CB | VAL | B | 47 | 22.282 | 25.237 | 2.421 | 1.00 | 31.81 | 6 | C |
| ATOM | 377 | CG1 | VAL | B | 47 | 23.752 | 25.408 | 2.700 | 1.00 | 28.39 | 6 | C |
| ATOM | 378 | CG2 | VAL | B | 47 | 21.819 | 23.806 | 2.521 | 1.00 | 32.58 | 6 | C |
| ATOM | 379 | N | ASP | B | 48 | 22.082 | 26.610 | 5.570 | 1.00 | 27.43 | 7 | N |
| ATOM | 380 | CA | ASP | B | 48 | 22.949 | 27.307 | 6.467 | 1.00 | 28.98 | 6 | C |
| ATOM | 381 | C | ASP | B | 48 | 24.229 | 26.461 | 6.527 | 1.00 | 26.95 | 6 | C |
| ATOM | 382 | O | ASP | B | 48 | 24.242 | 25.315 | 6.120 | 1.00 | 25.91 | 8 | O |
| ATOM | 383 | CB | ASP | B | 48 | 22.355 | 27.693 | 7.785 | 1.00 | 36.83 | 6 | C |
| ATOM | 384 | CG | ASP | B | 48 | 21.761 | 26.496 | 8.437 | 1.00 | 40.09 | 6 | C |
| ATOM | 385 | OD1 | ASP | B | 48 | 22.460 | 25.465 | 8.310 | 1.00 | 41.77 | 8 | O |
| ATOM | 386 | OD2 | ASP | B | 48 | 20.679 | 26.765 | 8.975 | 1.00 | 43.39 | 8 | O |
| ATOM | 387 | N | ILE | B | 49 | 25.356 | 26.998 | 6.922 | 1.00 | 25.50 | 7 | N |
| ATOM | 388 | CA | ILE | B | 49 | 26.617 | 26.370 | 7.024 | 1.00 | 26.25 | 6 | C |
| ATOM | 389 | C | ILE | B | 49 | 27.211 | 26.290 | 8.428 | 1.00 | 29.61 | 6 | C |
| ATOM | 390 | O | ILE | B | 49 | 27.399 | 27.368 | 9.031 | 1.00 | 33.54 | 8 | O |
| ATOM | 391 | CB | ILE | B | 49 | 27.515 | 27.271 | 6.155 | 1.00 | 28.15 | 6 | C |
| ATOM | 392 | CG1 | ILE | B | 49 | 27.180 | 27.048 | 4.655 | 1.00 | 26.64 | 6 | C |
| ATOM | 393 | CG2 | ILE | B | 49 | 29.013 | 26.939 | 6.341 | 1.00 | 29.92 | 6 | C |
| ATOM | 394 | CD1 | ILE | B | 49 | 27.758 | 28.238 | 3.847 | 1.00 | 24.01 | 6 | C |
| ATOM | 395 | N | THR | B | 50 | 27.536 | 25.067 | 8.922 | 1.00 | 27.22 | 7 | N |
| ATOM | 396 | CA | THR | B | 50 | 28.192 | 25.110 | 10.237 | 1.00 | 24.36 | 6 | C |
| ATOM | 397 | C | THR | B | 50 | 29.654 | 24.951 | 9.864 | 1.00 | 26.40 | 6 | C |
| ATOM | 398 | O | THR | B | 50 | 29.987 | 23.808 | 9.510 | 1.00 | 28.23 | 8 | O |
| ATOM | 399 | CB | THR | B | 50 | 27.636 | 23.979 | 11.081 | 1.00 | 25.29 | 6 | C |
| ATOM | 400 | OG1 | THR | B | 50 | 26.242 | 24.305 | 11.215 | 1.00 | 20.14 | 8 | O |
| ATOM | 401 | CG2 | THR | B | 50 | 28.382 | 23.735 | 12.402 | 1.00 | 23.12 | 6 | C |
| ATOM | 402 | N | LEU | B | 51 | 30.464 | 26.025 | 9.937 | 1.00 | 23.35 | 7 | N |
| ATOM | 403 | CA | LEU | B | 51 | 31.879 | 25.866 | 9.591 | 1.00 | 21.01 | 6 | C |
| ATOM | 404 | C | LEU | B | 51 | 32.720 | 25.635 | 10.833 | 1.00 | 23.77 | 6 | C |
| ATOM | 405 | O | LEU | B | 51 | 32.772 | 26.538 | 11.661 | 1.00 | 26.42 | 8 | O |
| ATOM | 406 | CB | LEU | B | 51 | 32.183 | 27.181 | 8.870 | 1.00 | 15.86 | 6 | C |
| ATOM | 407 | CG | LEU | B | 51 | 33.604 | 27.500 | 8.523 | 1.00 | 19.48 | 6 | C |
| ATOM | 408 | CD1 | LEU | B | 51 | 34.266 | 26.279 | 7.879 | 1.00 | 21.83 | 6 | C |
| ATOM | 409 | CD2 | LEU | B | 51 | 33.781 | 28.745 | 7.606 | 1.00 | 15.47 | 6 | C |
| ATOM | 410 | N | LEU | B | 52 | 33.409 | 24.502 | 11.022 | 1.00 | 23.52 | 7 | N |
| ATOM | 411 | CA | LEU | B | 52 | 34.222 | 24.247 | 12.225 | 1.00 | 20.46 | 6 | C |
| ATOM | 412 | C | LEU | B | 52 | 35.681 | 24.624 | 12.050 | 1.00 | 19.76 | 6 | C |
| ATOM | 413 | O | LEU | B | 52 | 36.148 | 24.765 | 10.907 | 1.00 | 19.49 | 8 | O |
| ATOM | 414 | CB | LEU | B | 52 | 34.094 | 22.749 | 12.464 | 1.00 | 20.38 | 6 | C |
| ATOM | 415 | CG | LEU | B | 52 | 32.708 | 22.210 | 12.478 | 1.00 | 22.77 | 6 | C |
| ATOM | 416 | CD1 | LEU | B | 52 | 32.665 | 20.671 | 12.699 | 1.00 | 22.76 | 6 | C |
| ATOM | 417 | CD2 | LEU | B | 52 | 32.125 | 22.835 | 13.789 | 1.00 | 25.84 | 6 | C |
| ATOM | 418 | N | PRO | B | 53 | 36.412 | 24.806 | 13.145 | 1.00 | 19.46 | 7 | N |
| ATOM | 419 | CA | PRO | B | 53 | 37.758 | 25.253 | 13.186 | 1.00 | 21.26 | 6 | C |
| ATOM | 420 | C | PRO | B | 53 | 38.726 | 24.150 | 12.836 | 1.00 | 23.87 | 6 | C |
| ATOM | 421 | O | PRO | B | 53 | 38.347 | 22.969 | 12.910 | 1.00 | 25.13 | 8 | O |
| ATOM | 422 | CB | PRO | B | 53 | 37.932 | 25.951 | 14.534 | 1.00 | 19.11 | 6 | C |
| ATOM | 423 | CG | PRO | B | 53 | 36.997 | 25.201 | 15.394 | 1.00 | 22.76 | 6 | C |
| ATOM | 424 | CD | PRO | B | 53 | 35.842 | 24.747 | 14.518 | 1.00 | 22.84 | 6 | C |
| ATOM | 425 | N | PRO | B | 54 | 39.878 | 24.558 | 12.390 | 1.00 | 25.90 | 7 | N |
| ATOM | 426 | CA | PRO | B | 54 | 40.930 | 23.646 | 12.023 | 1.00 | 28.49 | 6 | C |
| ATOM | 427 | C | PRO | B | 54 | 41.344 | 22.889 | 13.256 | 1.00 | 33.78 | 6 | C |
| ATOM | 428 | O | PRO | B | 54 | 41.152 | 23.396 | 14.380 | 1.00 | 36.09 | 8 | O |
| ATOM | 429 | CB | PRO | B | 54 | 42.022 | 24.566 | 11.555 | 1.00 | 27.21 | 6 | C |
| ATOM | 430 | CG | PRO | B | 54 | 41.760 | 25.960 | 12.048 | 1.00 | 27.82 | 6 | C |
| ATOM | 431 | CD | PRO | B | 54 | 40.303 | 26.001 | 12.347 | 1.00 | 26.80 | 6 | C |
| ATOM | 432 | N | ASP | B | 55 | 41.847 | 21.671 | 13.064 | 1.00 | 36.13 | 7 | N |
| ATOM | 433 | CA | ASP | B | 55 | 42.324 | 20.841 | 14.165 | 1.00 | 34.16 | 6 | C |
| ATOM | 434 | C | ASP | B | 55 | 43.637 | 20.328 | 13.636 | 1.00 | 35.50 | 6 | C |
| ATOM | 435 | O | ASP | B | 55 | 43.585 | 19.462 | 12.772 | 1.00 | 38.97 | 8 | O |
| ATOM | 436 | CB | ASP | B | 55 | 41.377 | 19.757 | 14.645 | 1.00 | 33.74 | 6 | C |
| ATOM | 437 | CG | ASP | B | 55 | 41.954 | 19.150 | 15.919 | 1.00 | 34.18 | 6 | C |
| ATOM | 438 | OD1 | ASP | B | 55 | 43.197 | 19.005 | 15.995 | 1.00 | 38.50 | 8 | O |
| ATOM | 439 | OD2 | ASP | B | 55 | 41.275 | 18.799 | 16.863 | 1.00 | 36.49 | 8 | O |
| ATOM | 440 | N | GLU | B | 56 | 44.723 | 20.857 | 14.052 | 1.00 | 36.17 | 7 | N |
| ATOM | 441 | CA | GLU | B | 56 | 46.085 | 20.556 | 13.624 | 1.00 | 37.43 | 6 | C |
| ATOM | 442 | C | GLU | B | 56 | 46.457 | 19.120 | 13.888 | 1.00 | 37.76 | 6 | C |
| ATOM | 443 | O | GLU | B | 56 | 47.169 | 18.489 | 13.107 | 1.00 | 38.26 | 8 | O |
| ATOM | 444 | CB | GLU | B | 56 | 47.028 | 21.583 | 14.217 | 1.00 | 37.41 | 6 | C |
| ATOM | 445 | CG | GLU | B | 56 | 47.041 | 22.930 | 13.529 | 1.00 | 35.74 | 6 | C |
| ATOM | 446 | CD | GLU | B | 56 | 47.339 | 22.949 | 12.055 | 1.00 | 39.31 | 6 | C |
| ATOM | 447 | OE1 | GLU | B | 56 | 47.995 | 22.144 | 11.348 | 1.00 | 37.10 | 8 | O |
| ATOM | 448 | OE2 | GLU | B | 56 | 46.842 | 23.945 | 11.443 | 1.00 | 41.28 | 8 | O |
| ATOM | 449 | N | ARG | B | 57 | 45.913 | 18.450 | 14.872 | 1.00 | 36.64 | 7 | N |
| ATOM | 450 | CA | ARG | B | 57 | 46.224 | 17.067 | 15.105 | 1.00 | 36.77 | 6 | C |
| ATOM | 451 | C | ARG | B | 57 | 45.657 | 16.173 | 13.992 | 1.00 | 38.45 | 6 | C |
| ATOM | 452 | O | ARG | B | 57 | 45.937 | 14.955 | 14.043 | 1.00 | 35.98 | 8 | O |
| ATOM | 453 | CB | ARG | B | 57 | 45.463 | 16.665 | 16.357 | 1.00 | 37.75 | 6 | C |
| ATOM | 454 | CG | ARG | B | 57 | 45.857 | 17.161 | 17.730 | 1.00 | 39.93 | 6 | C |
| ATOM | 455 | CD | ARG | B | 57 | 44.934 | 16.289 | 18.680 | 1.00 | 43.05 | 6 | C |
| ATOM | 456 | NE | ARG | B | 57 | 43.707 | 17.050 | 18.665 | 1.00 | 43.89 | 7 | N |
| ATOM | 457 | CZ | ARG | B | 57 | 42.669 | 16.850 | 19.474 | 1.00 | 48.81 | 6 | C |
| ATOM | 458 | NH1 | ARG | B | 57 | 42.924 | 15.824 | 20.320 | 1.00 | 49.04 | 7 | N |
| ATOM | 459 | NH2 | ARG | B | 57 | 41.548 | 17.629 | 19.385 | 1.00 | 43.97 | 7 | N |
| ATOM | 460 | N | PHE | B | 58 | 44.794 | 16.614 | 13.061 | 1.00 | 35.98 | 7 | N |
| ATOM | 461 | CA | PHE | B | 58 | 44.181 | 15.779 | 12.056 | 1.00 | 31.46 | 6 | C |
| ATOM | 462 | C | PHE | B | 58 | 44.216 | 16.441 | 10.688 | 1.00 | 32.33 | 6 | C |
| ATOM | 463 | O | PHE | B | 58 | 43.282 | 17.062 | 10.210 | 1.00 | 33.93 | 8 | O |
| ATOM | 464 | CB | PHE | B | 58 | 42.740 | 15.512 | 12.472 | 1.00 | 32.61 | 6 | C |
| ATOM | 465 | CG | PHE | B | 58 | 42.598 | 14.868 | 13.834 | 1.00 | 34.37 | 6 | C |
| ATOM | 466 | CD1 | PHE | B | 58 | 42.144 | 15.523 | 14.932 | 1.00 | 34.10 | 6 | C |
| ATOM | 467 | CD2 | PHE | B | 58 | 42.953 | 13.535 | 14.002 | 1.00 | 36.23 | 6 | C |
| ATOM | 468 | CE1 | PHE | B | 58 | 42.027 | 14.927 | 16.157 | 1.00 | 35.92 | 6 | C |
| ATOM | 469 | CE2 | PHE | B | 58 | 42.836 | 12.922 | 15.244 | 1.00 | 36.05 | 6 | C |
| ATOM | 470 | CZ | PHE | B | 58 | 42.375 | 13.606 | 16.336 | 1.00 | 35.52 | 6 | C |
| ATOM | 471 | N | PRO | B | 59 | 45.258 | 16.233 | 9.899 | 1.00 | 31.81 | 7 | N |
| ATOM | 472 | CA | PRO | B | 59 | 45.495 | 16.763 | 8.596 | 1.00 | 30.40 | 6 | C |
| ATOM | 473 | C | PRO | B | 59 | 44.426 | 16.644 | 7.553 | 1.00 | 33.46 | 6 | C |
| ATOM | 474 | O | PRO | B | 59 | 44.442 | 17.431 | 6.597 | 1.00 | 34.65 | 8 | O |
| ATOM | 475 | CB | PRO | B | 59 | 46.697 | 15.935 | 8.133 | 1.00 | 27.43 | 6 | C |
| ATOM | 476 | CG | PRO | B | 59 | 47.474 | 15.468 | 9.308 | 1.00 | 24.40 | 6 | C |
| ATOM | 477 | CD | PRO | B | 59 | 46.440 | 15.362 | 10.354 | 1.00 | 31.31 | 6 | C |
| ATOM | 478 | N | ASP | B | 60 | 43.585 | 15.617 | 7.598 | 1.00 | 32.30 | 7 | N |
| ATOM | 479 | CA | ASP | B | 60 | 42.575 | 15.368 | 6.593 | 1.00 | 32.25 | 6 | C |
| ATOM | 480 | C | ASP | B | 60 | 41.229 | 15.696 | 7.209 | 1.00 | 31.41 | 6 | C |
| ATOM | 481 | O | ASP | B | 60 | 40.179 | 15.461 | 6.613 | 1.00 | 30.80 | 8 | O |
| ATOM | 482 | CB | ASP | B | 60 | 42.652 | 13.894 | 6.095 | 1.00 | 35.82 | 6 | C |
| ATOM | 483 | CG | ASP | B | 60 | 43.699 | 13.539 | 5.019 | 1.00 | 41.10 | 6 | C |
| ATOM | 484 | OD1 | ASP | B | 60 | 43.750 | 13.963 | 3.855 | 1.00 | 42.33 | 8 | O |
| ATOM | 485 | OD2 | ASP | B | 60 | 44.625 | 12.712 | 5.351 | 1.00 | 43.52 | 8 | O |
| ATOM | 486 | N | SER | B | 61 | 41.099 | 16.314 | 8.409 | 1.00 | 29.05 | 7 | N |
| ATOM | 487 | CA | SER | B | 61 | 39.859 | 16.614 | 9.060 | 1.00 | 27.80 | 6 | C |
| ATOM | 488 | C | SER | B | 61 | 39.018 | 17.591 | 8.247 | 1.00 | 25.25 | 6 | C |
| ATOM | 489 | O | SER | B | 61 | 37.807 | 17.711 | 8.480 | 1.00 | 25.86 | 8 | O |
| ATOM | 490 | CB | SER | B | 61 | 39.948 | 17.063 | 10.552 | 1.00 | 28.51 | 6 | C |
| ATOM | 491 | OG | SER | B | 61 | 40.869 | 18.182 | 10.508 | 1.00 | 30.78 | 8 | O |
| ATOM | 492 | N | VAL | B | 62 | 39.501 | 18.239 | 7.243 | 1.00 | 25.32 | 7 | N |
| ATOM | 493 | CA | VAL | B | 62 | 38.796 | 19.101 | 6.339 | 1.00 | 23.96 | 6 | C |
| ATOM | 494 | C | VAL | B | 62 | 37.884 | 18.139 | 5.591 | 1.00 | 27.59 | 6 | C |
| ATOM | 495 | O | VAL | B | 62 | 36.827 | 18.606 | 5.133 | 1.00 | 29.34 | 8 | O |
| ATOM | 496 | CB | VAL | B | 62 | 39.739 | 19.693 | 5.266 | 1.00 | 25.54 | 6 | C |
| ATOM | 497 | CG1 | VAL | B | 62 | 40.432 | 18.640 | 4.437 | 1.00 | 22.14 | 6 | C |
| ATOM | 498 | CG2 | VAL | B | 62 | 39.019 | 20.639 | 4.294 | 1.00 | 22.52 | 6 | C |
| ATOM | 499 | N | PHE | B | 63 | 38.171 | 16.833 | 5.455 | 1.00 | 26.99 | 7 | N |
| ATOM | 500 | CA | PHE | B | 63 | 37.238 | 15.947 | 4.741 | 1.00 | 28.20 | 6 | C |
| ATOM | 501 | C | PHE | B | 63 | 36.190 | 15.397 | 5.680 | 1.00 | 29.66 | 6 | C |
| ATOM | 502 | O | PHE | B | 63 | 36.043 | 14.211 | 6.074 | 1.00 | 30.88 | 8 | O |
| ATOM | 503 | CB | PHE | B | 63 | 38.154 | 14.914 | 4.080 | 1.00 | 28.53 | 6 | C |
| ATOM | 504 | CG | PHE | B | 63 | 38.967 | 15.393 | 2.915 | 1.00 | 28.72 | 6 | C |
| ATOM | 505 | CD1 | PHE | B | 63 | 40.267 | 14.926 | 2.792 | 1.00 | 30.01 | 6 | C |
| ATOM | 506 | CD2 | PHE | B | 63 | 38.567 | 16.243 | 1.961 | 1.00 | 28.37 | 6 | C |
| ATOM | 507 | CE1 | PHE | B | 63 | 41.106 | 15.277 | 1.784 | 1.00 | 28.57 | 6 | C |
| ATOM | 508 | CE2 | PHE | B | 63 | 39.326 | 16.667 | 0.902 | 1.00 | 29.15 | 6 | C |
| ATOM | 509 | CZ | PHE | B | 63 | 40.599 | 16.151 | 0.854 | 1.00 | 32.62 | 6 | C |
| ATOM | 510 | N | VAL | B | 64 | 35.266 | 16.246 | 6.132 | 1.00 | 29.17 | 7 | N |
| ATOM | 511 | CA | VAL | B | 64 | 34.233 | 15.906 | 7.138 | 1.00 | 28.69 | 6 | C |
| ATOM | 512 | C | VAL | B | 64 | 33.287 | 14.892 | 6.605 | 1.00 | 28.04 | 6 | C |
| ATOM | 513 | O | VAL | B | 64 | 32.539 | 14.293 | 7.376 | 1.00 | 28.00 | 8 | O |
| ATOM | 514 | CB | VAL | B | 64 | 33.499 | 17.124 | 7.807 | 1.00 | 30.24 | 6 | C |
| ATOM | 515 | CG1 | VAL | B | 64 | 34.597 | 18.113 | 8.251 | 1.00 | 32.67 | 6 | C |
| ATOM | 516 | CG2 | VAL | B | 64 | 32.584 | 17.874 | 6.861 | 1.00 | 27.31 | 6 | C |
| ATOM | 517 | N | GLU | B | 65 | 33.214 | 14.633 | 5.295 | 1.00 | 28.08 | 7 | N |
| ATOM | 518 | CA | GLU | B | 65 | 32.302 | 13.568 | 4.811 | 1.00 | 26.34 | 6 | C |
| ATOM | 519 | C | GLU | B | 65 | 32.709 | 12.218 | 5.414 | 1.00 | 27.29 | 6 | C |
| ATOM | 520 | O | GLU | B | 65 | 31.775 | 11.470 | 5.657 | 1.00 | 26.81 | 8 | O |
| ATOM | 521 | CB | GLU | B | 65 | 32.369 | 13.427 | 3.288 | 1.00 | 27.27 | 6 | C |
| ATOM | 522 | CG | GLU | B | 65 | 31.980 | 12.129 | 2.614 | 1.00 | 28.06 | 6 | C |
| ATOM | 523 | CD | GLU | B | 65 | 31.652 | 12.280 | 1.132 | 1.00 | 29.42 | 6 | C |
| ATOM | 524 | OE1 | GLU | B | 65 | 32.535 | 12.066 | 0.241 | 1.00 | 33.67 | 8 | O |
| ATOM | 525 | OE2 | GLU | B | 65 | 30.530 | 12.625 | 0.736 | 1.00 | 27.39 | 8 | O |
| ATOM | 526 | N | ASP | B | 66 | 34.012 | 11.901 | 5.728 | 1.00 | 27.65 | 7 | N |
| ATOM | 527 | CA | ASP | B | 66 | 34.288 | 10.572 | 6.234 | 1.00 | 27.31 | 6 | C |
| ATOM | 528 | C | ASP | B | 66 | 33.805 | 10.142 | 7.556 | 1.00 | 29.71 | 6 | C |
| ATOM | 529 | O | ASP | B | 66 | 33.232 | 9.046 | 7.600 | 1.00 | 31.57 | 8 | O |
| ATOM | 530 | CB | ASP | B | 66 | 35.752 | 10.153 | 6.004 | 1.00 | 29.68 | 6 | C |
| ATOM | 531 | CG | ASP | B | 66 | 35.878 | 10.185 | 4.467 | 1.00 | 30.27 | 6 | C |
| ATOM | 532 | OD1 | ASP | B | 66 | 35.073 | 9.586 | 3.707 | 1.00 | 29.40 | 8 | O |
| ATOM | 533 | OD2 | ASP | B | 66 | 36.831 | 10.863 | 4.067 | 1.00 | 32.32 | 8 | O |
| ATOM | 534 | N | PRO | B | 67 | 33.895 | 10.888 | 8.620 | 1.00 | 32.42 | 7 | N |
| ATOM | 535 | CA | PRO | B | 67 | 33.492 | 10.452 | 9.943 | 1.00 | 29.38 | 6 | C |
| ATOM | 536 | C | PRO | B | 67 | 32.069 | 10.470 | 10.300 | 1.00 | 31.31 | 6 | C |
| ATOM | 537 | O | PRO | B | 67 | 31.721 | 10.152 | 11.456 | 1.00 | 34.21 | 8 | O |
| ATOM | 538 | CB | PRO | B | 67 | 34.336 | 11.369 | 10.847 | 1.00 | 32.40 | 6 | C |
| ATOM | 539 | CG | PRO | B | 67 | 34.256 | 12.637 | 10.050 | 1.00 | 32.58 | 6 | C |
| ATOM | 540 | CD | PRO | B | 67 | 34.617 | 12.208 | 8.646 | 1.00 | 32.58 | 6 | C |
| ATOM | 541 | N | VAL | B | 68 | 31.031 | 10.705 | 9.533 | 1.00 | 31.12 | 7 | N |
| ATOM | 542 | CA | VAL | B | 68 | 29.665 | 10.709 | 9.949 | 1.00 | 30.61 | 6 | C |
| ATOM | 543 | C | VAL | B | 68 | 28.770 | 10.515 | 8.714 | 1.00 | 30.18 | 6 | C |
| ATOM | 544 | O | VAL | B | 68 | 29.157 | 11.104 | 7.756 | 1.00 | 29.48 | 8 | O |
| ATOM | 545 | CB | VAL | B | 68 | 29.079 | 12.101 | 10.461 | 1.00 | 29.80 | 6 | C |
| ATOM | 546 | CG1 | VAL | B | 68 | 27.757 | 11.920 | 11.257 | 1.00 | 24.42 | 6 | C |
| ATOM | 547 | CG2 | VAL | B | 68 | 30.096 | 12.931 | 11.174 | 1.00 | 27.14 | 6 | C |
| ATOM | 548 | N | LEU | B | 69 | 27.667 | 9.821 | 8.829 | 1.00 | 29.80 | 7 | N |
| ATOM | 549 | CA | LEU | B | 69 | 26.757 | 9.710 | 7.715 | 1.00 | 31.09 | 6 | C |
| ATOM | 550 | C | LEU | B | 69 | 25.515 | 10.454 | 8.223 | 1.00 | 32.12 | 6 | C |
| ATOM | 551 | O | LEU | B | 69 | 25.094 | 10.024 | 9.331 | 1.00 | 33.34 | 8 | O |
| ATOM | 552 | CB | LEU | B | 69 | 26.315 | 8.288 | 7.402 | 1.00 | 29.41 | 6 | C |
| ATOM | 553 | CG | LEU | B | 69 | 25.336 | 8.098 | 6.233 | 1.00 | 27.88 | 6 | C |
| ATOM | 554 | CD1 | LEU | B | 69 | 26.036 | 8.651 | 5.037 | 1.00 | 24.06 | 6 | C |
| ATOM | 555 | CD2 | LEU | B | 69 | 24.842 | 6.669 | 6.044 | 1.00 | 26.77 | 6 | C |
| ATOM | 556 | N | CYS | B | 70 | 25.055 | 11.475 | 7.503 | 1.00 | 28.60 | 7 | N |
| ATOM | 557 | CA | CYS | B | 70 | 23.872 | 12.165 | 8.057 | 1.00 | 31.55 | 6 | C |
| ATOM | 558 | C | CYS | B | 70 | 22.657 | 11.772 | 7.237 | 1.00 | 33.58 | 6 | C |
| ATOM | 559 | O | CYS | B | 70 | 22.688 | 12.055 | 6.046 | 1.00 | 33.52 | 8 | O |
| ATOM | 560 | CB | CYS | B | 70 | 23.974 | 13.697 | 8.020 | 1.00 | 29.40 | 6 | C |
| ATOM | 561 | SG | CYS | B | 70 | 25.279 | 14.187 | 9.105 | 1.00 | 28.56 | 16 | S |
| ATOM | 562 | N | THR | B | 71 | 21.637 | 11.187 | 7.857 | 1.00 | 36.16 | 7 | N |
| ATOM | 563 | CA | THR | B | 71 | 20.465 | 10.806 | 7.037 | 1.00 | 38.64 | 6 | C |
| ATOM | 564 | C | THR | B | 71 | 19.293 | 11.640 | 7.496 | 1.00 | 39.50 | 6 | C |
| ATOM | 565 | O | THR | B | 71 | 19.424 | 12.566 | 8.304 | 1.00 | 39.95 | 8 | O |
| ATOM | 566 | CB | THR | B | 71 | 20.205 | 9.278 | 7.164 | 1.00 | 38.71 | 6 | C |
| ATOM | 567 | OG1 | THR | B | 71 | 19.707 | 8.976 | 8.465 | 1.00 | 38.17 | 8 | O |
| ATOM | 568 | CG2 | THR | B | 71 | 21.532 | 8.534 | 7.001 | 1.00 | 37.87 | 6 | C |
| ATOM | 569 | N | SER | B | 72 | 18.074 | 11.330 | 7.093 | 1.00 | 38.97 | 7 | N |
| ATOM | 570 | CA | SER | B | 72 | 16.926 | 12.153 | 7.456 | 1.00 | 36.76 | 6 | C |
| ATOM | 571 | C | SER | B | 72 | 16.267 | 11.648 | 8.726 | 1.00 | 40.40 | 6 | C |
| ATOM | 572 | O | SER | B | 72 | 15.236 | 12.211 | 9.155 | 1.00 | 40.70 | 8 | O |
| ATOM | 573 | CB | SER | B | 72 | 15.845 | 12.097 | 6.382 | 1.00 | 33.75 | 6 | C |
| ATOM | 574 | OG | SER | B | 72 | 15.656 | 10.697 | 6.364 | 1.00 | 39.66 | 8 | O |
| ATOM | 575 | N | ARG | B | 73 | 16.872 | 10.567 | 9.279 | 1.00 | 41.30 | 7 | N |
| ATOM | 576 | CA | ARG | B | 73 | 16.336 | 9.983 | 10.511 | 1.00 | 40.24 | 6 | C |
| ATOM | 577 | C | ARG | B | 73 | 17.366 | 9.859 | 11.583 | 1.00 | 40.33 | 6 | C |
| ATOM | 578 | O | ARG | B | 73 | 16.865 | 9.888 | 12.704 | 1.00 | 44.89 | 8 | O |
| ATOM | 579 | CB | ARG | B | 73 | 15.649 | 8.642 | 10.250 | 1.00 | 40.64 | 6 | C |
| ATOM | 580 | CG | ARG | B | 73 | 14.533 | 8.909 | 9.220 | 1.00 | 43.07 | 6 | C |
| ATOM | 581 | CD | ARG | B | 73 | 13.381 | 8.031 | 9.105 | 1.00 | 46.97 | 6 | C |
| ATOM | 582 | NE | ARG | B | 73 | 12.878 | 7.954 | 7.720 | 1.00 | 52.51 | 7 | N |
| ATOM | 583 | CZ | ARG | B | 73 | 12.508 | 6.739 | 7.269 | 1.00 | 54.93 | 6 | C |
| ATOM | 584 | NH1 | ARG | B | 73 | 12.595 | 5.645 | 8.042 | 1.00 | 56.12 | 7 | N |
| ATOM | 585 | NH2 | ARG | B | 73 | 12.042 | 6.576 | 6.035 | 1.00 | 57.41 | 7 | N |
| ATOM | 586 | N | CYS | B | 74 | 18.650 | 9.820 | 11.357 | 1.00 | 36.47 | 7 | N |
| ATOM | 587 | CA | CYS | B | 74 | 19.622 | 9.688 | 12.425 | 1.00 | 36.75 | 6 | C |
| ATOM | 588 | C | CYS | B | 74 | 20.909 | 10.138 | 11.730 | 1.00 | 35.64 | 6 | C |
| ATOM | 589 | O | CYS | B | 74 | 20.773 | 10.460 | 10.564 | 1.00 | 38.53 | 8 | O |
| ATOM | 590 | CB | CYS | B | 74 | 19.909 | 8.216 | 12.834 | 1.00 | 37.46 | 6 | C |
| ATOM | 591 | SG | CYS | B | 74 | 20.136 | 7.115 | 11.380 | 1.00 | 37.59 | 16 | S |
| ATOM | 592 | N | ALA | B | 75 | 22.000 | 10.071 | 12.404 | 1.00 | 33.17 | 7 | N |
| ATOM | 593 | CA | ALA | B | 75 | 23.329 | 10.369 | 12.023 | 1.00 | 33.65 | 6 | C |
| ATOM | 594 | C | ALA | B | 75 | 24.159 | 9.175 | 12.596 | 1.00 | 34.81 | 6 | C |
| ATOM | 595 | O | ALA | B | 75 | 23.802 | 8.789 | 13.704 | 1.00 | 34.11 | 8 | O |
| ATOM | 596 | CB | ALA | B | 75 | 23.852 | 11.658 | 12.649 | 1.00 | 26.62 | 6 | C |
| ATOM | 597 | N | ILE | B | 76 | 25.145 | 8.654 | 11.824 | 1.00 | 31.15 | 7 | N |
| ATOM | 598 | CA | ILE | B | 76 | 25.901 | 7.552 | 12.351 | 1.00 | 29.78 | 6 | C |
| ATOM | 599 | C | ILE | B | 76 | 27.343 | 8.012 | 12.444 | 1.00 | 30.84 | 6 | C |
| ATOM | 600 | O | ILE | B | 76 | 27.771 | 8.411 | 11.347 | 1.00 | 33.08 | 8 | O |
| ATOM | 601 | CB | ILE | B | 76 | 25.790 | 6.265 | 11.483 | 1.00 | 28.63 | 6 | C |
| ATOM | 602 | CG1 | ILE | B | 76 | 24.348 | 5.715 | 11.492 | 1.00 | 25.35 | 6 | C |
| ATOM | 603 | CG2 | ILE | B | 76 | 26.709 | 5.113 | 11.959 | 1.00 | 26.69 | 6 | C |
| ATOM | 604 | CD1 | ILE | B | 76 | 23.539 | 6.458 | 10.420 | 1.00 | 22.89 | 6 | C |
| ATOM | 605 | N | ILE | B | 77 | 27.956 | 8.056 | 13.592 | 1.00 | 30.09 | 7 | N |
| ATOM | 606 | CA | ILE | B | 77 | 29.341 | 8.464 | 13.727 | 1.00 | 29.98 | 6 | C |
| ATOM | 607 | C | ILE | B | 77 | 30.075 | 7.299 | 13.103 | 1.00 | 34.01 | 6 | C |
| ATOM | 608 | O | ILE | B | 77 | 29.820 | 6.189 | 13.579 | 1.00 | 39.53 | 8 | O |
| ATOM | 609 | CB | ILE | B | 77 | 29.716 | 8.686 | 15.162 | 1.00 | 26.31 | 6 | C |
| ATOM | 610 | CG1 | ILE | B | 77 | 28.970 | 9.812 | 15.892 | 1.00 | 31.61 | 6 | C |
| ATOM | 611 | CG2 | ILE | B | 77 | 31.159 | 8.992 | 15.247 | 1.00 | 23.83 | 6 | C |
| ATOM | 612 | CD1 | ILE | B | 77 | 28.823 | 11.124 | 15.162 | 1.00 | 36.62 | 6 | C |
| ATOM | 613 | N | THR | B | 78 | 30.860 | 7.449 | 12.064 | 1.00 | 34.45 | 7 | N |
| ATOM | 614 | CA | THR | B | 78 | 31.518 | 6.294 | 11.489 | 1.00 | 34.40 | 6 | C |
| ATOM | 615 | C | THR | B | 78 | 32.733 | 5.870 | 12.287 | 1.00 | 37.50 | 6 | C |
| ATOM | 616 | O | THR | B | 78 | 32.864 | 6.307 | 13.452 | 1.00 | 40.27 | 8 | O |
| ATOM | 617 | CB | THR | B | 78 | 31.863 | 6.664 | 10.022 | 1.00 | 35.34 | 6 | C |
| ATOM | 618 | OG1 | THR | B | 78 | 32.884 | 7.638 | 9.901 | 1.00 | 31.85 | 8 | O |
| ATOM | 619 | CG2 | THR | B | 78 | 30.604 | 7.019 | 9.264 | 1.00 | 32.16 | 6 | C |
| ATOM | 620 | N | ARG | B | 79 | 33.634 | 5.058 | 11.710 | 1.00 | 36.76 | 7 | N |
| ATOM | 621 | CA | ARG | B | 79 | 34.861 | 4.607 | 12.362 | 1.00 | 35.04 | 6 | C |
| ATOM | 622 | C | ARG | B | 79 | 35.860 | 4.327 | 11.232 | 1.00 | 36.44 | 6 | C |
| ATOM | 623 | O | ARG | B | 79 | 35.960 | 3.230 | 10.733 | 1.00 | 35.64 | 8 | O |
| ATOM | 624 | CB | ARG | B | 79 | 34.648 | 3.308 | 13.154 | 1.00 | 34.05 | 6 | C |
| ATOM | 625 | CG | ARG | B | 79 | 35.802 | 2.861 | 13.978 | 1.00 | 31.80 | 6 | C |
| ATOM | 626 | CD | ARG | B | 79 | 35.456 | 1.753 | 15.026 | 1.00 | 36.53 | 6 | C |
| ATOM | 627 | NE | ARG | B | 79 | 36.811 | 1.555 | 15.519 | 1.00 | 39.26 | 7 | N |
| ATOM | 628 | CZ | ARG | B | 79 | 37.696 | 0.661 | 15.112 | 1.00 | 41.66 | 6 | C |
| ATOM | 629 | NH1 | ARG | B | 79 | 37.233 | −0.126 | 14.154 | 1.00 | 39.87 | 7 | N |
| ATOM | 630 | NH2 | ARG | B | 79 | 38.913 | 0.672 | 15.720 | 1.00 | 43.81 | 7 | N |
| ATOM | 631 | N | PRO | B | 80 | 36.581 | 5.363 | 10.827 | 1.00 | 38.23 | 7 | N |
| ATOM | 632 | CA | PRO | B | 80 | 37.528 | 5.359 | 9.785 | 1.00 | 39.29 | 6 | C |
| ATOM | 633 | C | PRO | B | 80 | 38.592 | 4.243 | 9.846 | 1.00 | 39.91 | 6 | C |
| ATOM | 634 | O | PRO | B | 80 | 39.040 | 3.836 | 10.913 | 1.00 | 38.77 | 8 | O |
| ATOM | 635 | CB | PRO | B | 80 | 38.174 | 6.718 | 9.756 | 1.00 | 35.25 | 6 | C |
| ATOM | 636 | CG | PRO | B | 80 | 37.292 | 7.553 | 10.552 | 1.00 | 36.37 | 6 | C |
| ATOM | 637 | CD | PRO | B | 80 | 36.443 | 6.720 | 11.408 | 1.00 | 38.31 | 6 | C |
| ATOM | 638 | N | GLY | B | 81 | 38.902 | 3.915 | 8.572 | 1.00 | 39.92 | 7 | N |
| ATOM | 639 | CA | GLY | B | 81 | 39.874 | 2.848 | 8.402 | 1.00 | 43.26 | 6 | C |
| ATOM | 640 | C | GLY | B | 81 | 41.200 | 3.417 | 8.834 | 1.00 | 44.29 | 6 | C |
| ATOM | 641 | O | GLY | B | 81 | 41.753 | 2.697 | 9.667 | 1.00 | 45.90 | 8 | O |
| ATOM | 642 | N | ALA | B | 82 | 41.563 | 4.555 | 8.234 | 1.00 | 44.34 | 7 | N |
| ATOM | 643 | CA | ALA | B | 82 | 42.878 | 5.073 | 8.698 | 1.00 | 44.59 | 6 | C |
| ATOM | 644 | C | ALA | B | 82 | 42.780 | 5.397 | 10.176 | 1.00 | 47.27 | 6 | C |
| ATOM | 645 | O | ALA | B | 82 | 41.802 | 6.037 | 10.661 | 1.00 | 46.93 | 8 | O |
| ATOM | 646 | CB | ALA | B | 82 | 43.327 | 6.349 | 8.000 | 1.00 | 44.15 | 6 | C |
| ATOM | 647 | N | GLU | B | 83 | 43.838 | 4.974 | 10.886 | 1.00 | 49.39 | 7 | N |
| ATOM | 648 | CA | GLU | B | 83 | 43.835 | 5.246 | 12.339 | 1.00 | 50.04 | 6 | C |
| ATOM | 649 | C | GLU | B | 83 | 44.184 | 6.705 | 12.543 | 1.00 | 48.35 | 6 | C |
| ATOM | 650 | O | GLU | B | 83 | 43.818 | 7.193 | 13.607 | 1.00 | 50.09 | 8 | O |
| ATOM | 651 | CB | GLU | B | 83 | 44.877 | 4.461 | 13.156 | 1.00 | 54.31 | 6 | C |
| ATOM | 652 | CG | GLU | B | 83 | 44.952 | 4.589 | 14.616 | 0.00 | 25.00 | 6 | C |
| ATOM | 653 | CD | GLU | B | 83 | 45.959 | 3.701 | 15.321 | 0.00 | 25.00 | 6 | C |
| ATOM | 654 | OE1 | GLU | B | 83 | 46.636 | 2.909 | 14.634 | 0.00 | 25.00 | 8 | O |
| ATOM | 655 | OE2 | GLU | B | 83 | 46.070 | 3.797 | 16.562 | 0.00 | 25.00 | 8 | O |
| ATOM | 656 | N | SER | B | 84 | 44.866 | 7.388 | 11.643 | 1.00 | 45.62 | 7 | N |
| ATOM | 657 | CA | SER | B | 84 | 45.150 | 8.798 | 11.945 | 1.00 | 44.32 | 6 | C |
| ATOM | 658 | C | SER | B | 84 | 43.853 | 9.620 | 11.806 | 1.00 | 43.59 | 6 | C |
| ATOM | 659 | O | SER | B | 84 | 43.688 | 10.749 | 12.223 | 1.00 | 44.43 | 8 | O |
| ATOM | 660 | CB | SER | B | 84 | 46.187 | 9.340 | 10.942 | 1.00 | 41.75 | 6 | C |
| ATOM | 661 | OG | SER | B | 84 | 45.592 | 9.067 | 9.670 | 1.00 | 40.88 | 8 | O |
| ATOM | 662 | N | ARG | B | 85 | 42.791 | 9.122 | 11.207 | 1.00 | 42.21 | 7 | N |
| ATOM | 663 | CA | ARG | B | 85 | 41.574 | 9.856 | 11.035 | 1.00 | 40.36 | 6 | C |
| ATOM | 664 | C | ARG | B | 85 | 40.525 | 9.387 | 12.012 | 1.00 | 43.01 | 6 | C |
| ATOM | 665 | O | ARG | B | 85 | 39.387 | 9.881 | 12.065 | 1.00 | 43.24 | 8 | O |
| ATOM | 666 | CB | ARG | B | 85 | 41.080 | 9.857 | 9.621 | 1.00 | 36.83 | 6 | C |
| ATOM | 667 | CG | ARG | B | 85 | 42.139 | 10.107 | 8.563 | 1.00 | 30.39 | 6 | C |
| ATOM | 668 | CD | ARG | B | 85 | 41.462 | 10.353 | 7.219 | 1.00 | 33.58 | 6 | C |
| ATOM | 669 | NE | ARG | B | 85 | 40.263 | 11.191 | 7.518 | 1.00 | 37.12 | 7 | N |
| ATOM | 670 | CZ | ARG | B | 85 | 39.242 | 11.576 | 6.736 | 1.00 | 32.99 | 6 | C |
| ATOM | 671 | NH1 | ARG | B | 85 | 39.102 | 11.269 | 5.467 | 1.00 | 28.35 | 7 | N |
| ATOM | 672 | NH2 | ARG | B | 85 | 38.323 | 12.324 | 7.306 | 1.00 | 29.95 | 7 | N |
| ATOM | 673 | N | ARG | B | 86 | 40.953 | 8.448 | 12.857 | 1.00 | 43.02 | 7 | N |
| ATOM | 674 | CA | ARG | B | 86 | 39.938 | 7.954 | 13.793 | 1.00 | 44.16 | 6 | C |
| ATOM | 675 | C | ARG | B | 86 | 39.485 | 9.013 | 14.769 | 1.00 | 43.25 | 6 | C |
| ATOM | 676 | O | ARG | B | 86 | 38.295 | 9.219 | 15.050 | 1.00 | 41.23 | 8 | O |
| ATOM | 677 | CB | ARG | B | 86 | 40.477 | 6.655 | 14.419 | 1.00 | 48.03 | 6 | C |
| ATOM | 678 | CG | ARG | B | 86 | 39.367 | 5.922 | 15.181 | 1.00 | 50.46 | 6 | C |
| ATOM | 679 | CD | ARG | B | 86 | 39.546 | 4.429 | 15.097 | 1.00 | 53.10 | 6 | C |
| ATOM | 680 | NE | ARG | B | 86 | 39.781 | 3.952 | 13.716 | 1.00 | 55.19 | 7 | N |
| ATOM | 681 | CZ | ARG | B | 86 | 40.347 | 2.725 | 13.590 | 1.00 | 57.50 | 6 | C |
| ATOM | 682 | NH1 | ARG | B | 86 | 40.693 | 1.952 | 14.622 | 1.00 | 57.03 | 7 | N |
| ATOM | 683 | NH2 | ARG | B | 86 | 40.580 | 2.244 | 12.376 | 1.00 | 58.92 | 7 | N |
| ATOM | 684 | N | GLY | B | 87 | 40.438 | 9.760 | 15.365 | 1.00 | 42.69 | 7 | N |
| ATOM | 685 | CA | GLY | B | 87 | 40.013 | 10.829 | 16.313 | 1.00 | 39.83 | 6 | C |
| ATOM | 686 | C | GLY | B | 87 | 39.084 | 11.849 | 15.657 | 1.00 | 39.04 | 6 | C |
| ATOM | 687 | O | GLY | B | 87 | 38.337 | 12.516 | 16.398 | 1.00 | 39.11 | 8 | O |
| ATOM | 688 | N | GLU | B | 88 | 38.971 | 12.018 | 14.312 | 1.00 | 34.71 | 7 | N |
| ATOM | 689 | CA | GLU | B | 88 | 38.043 | 12.967 | 13.729 | 1.00 | 33.19 | 6 | C |
| ATOM | 690 | C | GLU | B | 88 | 36.633 | 12.690 | 14.140 | 1.00 | 34.80 | 6 | C |
| ATOM | 691 | O | GLU | B | 88 | 35.765 | 13.576 | 14.159 | 1.00 | 39.09 | 8 | O |
| ATOM | 692 | CB | GLU | B | 88 | 38.090 | 13.143 | 12.189 | 1.00 | 31.20 | 6 | C |
| ATOM | 693 | CG | GLU | B | 88 | 39.544 | 13.085 | 11.777 | 1.00 | 29.34 | 6 | C |
| ATOM | 694 | CD | GLU | B | 88 | 39.787 | 13.201 | 10.318 | 1.00 | 32.64 | 6 | C |
| ATOM | 695 | OE1 | GLU | B | 88 | 38.835 | 13.427 | 9.539 | 1.00 | 34.29 | 8 | O |
| ATOM | 696 | OE2 | GLU | B | 88 | 40.965 | 13.070 | 9.888 | 1.00 | 31.61 | 8 | O |
| ATOM | 697 | N | THR | B | 89 | 36.277 | 11.529 | 14.602 | 1.00 | 34.57 | 7 | N |
| ATOM | 698 | CA | THR | B | 89 | 34.935 | 11.273 | 15.099 | 1.00 | 35.38 | 6 | C |
| ATOM | 699 | C | THR | B | 89 | 34.769 | 12.003 | 16.419 | 1.00 | 35.89 | 6 | C |
| ATOM | 700 | O | THR | B | 89 | 33.575 | 12.273 | 16.706 | 1.00 | 38.88 | 8 | O |
| ATOM | 701 | CB | THR | B | 89 | 34.641 | 9.774 | 15.322 | 1.00 | 35.76 | 6 | C |
| ATOM | 702 | OG1 | THR | B | 89 | 35.489 | 9.096 | 16.277 | 1.00 | 43.26 | 8 | O |
| ATOM | 703 | CG2 | THR | B | 89 | 35.122 | 9.189 | 14.004 | 1.00 | 37.54 | 6 | C |
| ATOM | 704 | N | GLU | B | 90 | 35.775 | 12.245 | 17.246 | 1.00 | 35.28 | 7 | N |
| ATOM | 705 | CA | GLU | B | 90 | 35.493 | 12.823 | 18.568 | 1.00 | 40.16 | 6 | C |
| ATOM | 706 | C | GLU | B | 90 | 35.289 | 14.324 | 18.497 | 1.00 | 38.17 | 6 | C |
| ATOM | 707 | O | GLU | B | 90 | 34.648 | 14.926 | 19.332 | 1.00 | 38.54 | 8 | O |
| ATOM | 708 | CB | GLU | B | 90 | 36.609 | 12.423 | 19.569 | 1.00 | 42.50 | 6 | C |
| ATOM | 709 | CG | GLU | B | 90 | 36.573 | 10.981 | 19.988 | 1.00 | 53.93 | 6 | C |
| ATOM | 710 | CD | GLU | B | 90 | 37.638 | 10.489 | 20.956 | 1.00 | 62.24 | 6 | C |
| ATOM | 711 | OE1 | GLU | B | 90 | 38.672 | 11.205 | 21.177 | 1.00 | 67.37 | 8 | O |
| ATOM | 712 | OE2 | GLU | B | 90 | 37.552 | 9.383 | 21.566 | 1.00 | 65.45 | 8 | O |
| ATOM | 713 | N | ILE | B | 91 | 35.877 | 14.948 | 17.499 | 1.00 | 36.26 | 7 | N |
| ATOM | 714 | CA | ILE | B | 91 | 35.820 | 16.388 | 17.427 | 1.00 | 33.14 | 6 | C |
| ATOM | 715 | C | ILE | B | 91 | 34.668 | 16.816 | 16.573 | 1.00 | 34.91 | 6 | C |
| ATOM | 716 | O | ILE | B | 91 | 34.601 | 17.996 | 16.237 | 1.00 | 38.56 | 8 | O |
| ATOM | 717 | CB | ILE | B | 91 | 37.128 | 17.108 | 16.937 | 1.00 | 30.68 | 6 | C |
| ATOM | 718 | CG1 | ILE | B | 91 | 37.458 | 16.782 | 15.490 | 1.00 | 28.99 | 6 | C |
| ATOM | 719 | CG2 | ILE | B | 91 | 38.332 | 16.862 | 17.878 | 1.00 | 26.11 | 6 | C |
| ATOM | 720 | CD1 | ILE | B | 91 | 38.719 | 17.401 | 14.886 | 1.00 | 26.12 | 6 | C |
| ATOM | 721 | N | ILE | B | 92 | 33.779 | 15.941 | 16.125 | 1.00 | 33.56 | 7 | N |
| ATOM | 722 | CA | ILE | B | 92 | 32.713 | 16.404 | 15.261 | 1.00 | 30.05 | 6 | C |
| ATOM | 723 | C | ILE | B | 92 | 31.426 | 15.972 | 15.945 | 1.00 | 28.90 | 6 | C |
| ATOM | 724 | O | ILE | B | 92 | 30.378 | 16.501 | 15.568 | 1.00 | 31.74 | 8 | O |
| ATOM | 725 | CB | ILE | B | 92 | 32.748 | 15.846 | 13.827 | 1.00 | 31.59 | 6 | C |
| ATOM | 726 | CG1 | ILE | B | 92 | 31.798 | 16.709 | 12.926 | 1.00 | 30.23 | 6 | C |
| ATOM | 727 | CG2 | ILE | B | 92 | 32.117 | 14.437 | 13.736 | 1.00 | 27.81 | 6 | C |
| ATOM | 728 | CD1 | ILE | B | 92 | 32.167 | 16.803 | 11.482 | 1.00 | 30.78 | 6 | C |
| ATOM | 729 | N | GLU | B | 93 | 31.504 | 15.062 | 16.873 | 1.00 | 28.89 | 7 | N |
| ATOM | 730 | CA | GLU | B | 93 | 30.268 | 14.640 | 17.509 | 1.00 | 35.45 | 6 | C |
| ATOM | 731 | C | GLU | B | 93 | 29.491 | 15.712 | 18.240 | 1.00 | 34.44 | 6 | C |
| ATOM | 732 | O | GLU | B | 93 | 28.263 | 15.578 | 18.326 | 1.00 | 36.73 | 8 | O |
| ATOM | 733 | CB | GLU | B | 93 | 30.598 | 13.525 | 18.518 | 1.00 | 40.07 | 6 | C |
| ATOM | 734 | CG | GLU | B | 93 | 29.353 | 12.765 | 18.918 | 1.00 | 44.70 | 6 | C |
| ATOM | 735 | CD | GLU | B | 93 | 29.711 | 11.391 | 19.469 | 1.00 | 50.92 | 6 | C |
| ATOM | 736 | OE1 | GLU | B | 93 | 30.827 | 10.856 | 19.155 | 1.00 | 51.61 | 8 | O |
| ATOM | 737 | OE2 | GLU | B | 93 | 28.782 | 10.921 | 20.223 | 1.00 | 53.15 | 8 | O |
| ATOM | 738 | N | GLU | B | 94 | 30.090 | 16.741 | 18.773 | 1.00 | 34.39 | 7 | N |
| ATOM | 739 | CA | GLU | B | 94 | 29.265 | 17.705 | 19.514 | 1.00 | 38.62 | 6 | C |
| ATOM | 740 | C | GLU | B | 94 | 28.196 | 18.274 | 18.557 | 1.00 | 36.04 | 6 | C |
| ATOM | 741 | O | GLU | B | 94 | 27.006 | 18.293 | 18.757 | 1.00 | 31.38 | 8 | O |
| ATOM | 742 | CB | GLU | B | 94 | 30.195 | 18.606 | 20.292 | 1.00 | 40.91 | 6 | C |
| ATOM | 743 | CG | GLU | B | 94 | 29.427 | 19.538 | 21.225 | 1.00 | 48.97 | 6 | C |
| ATOM | 744 | CD | GLU | B | 94 | 30.417 | 20.417 | 22.006 | 1.00 | 55.09 | 6 | C |
| ATOM | 745 | OE1 | GLU | B | 94 | 31.206 | 19.903 | 22.827 | 1.00 | 55.37 | 8 | O |
| ATOM | 746 | OE2 | GLU | B | 94 | 30.365 | 21.664 | 21.751 | 1.00 | 60.55 | 8 | O |
| ATOM | 747 | N | THR | B | 95 | 28.660 | 18.737 | 17.393 | 1.00 | 34.73 | 7 | N |
| ATOM | 748 | CA | THR | B | 95 | 27.929 | 19.329 | 16.278 | 1.00 | 31.45 | 6 | C |
| ATOM | 749 | C | THR | B | 95 | 26.889 | 18.341 | 15.841 | 1.00 | 32.92 | 6 | C |
| ATOM | 750 | O | THR | B | 95 | 25.719 | 18.732 | 15.765 | 1.00 | 35.08 | 8 | O |
| ATOM | 751 | CB | THR | B | 95 | 28.942 | 19.517 | 15.122 | 1.00 | 31.31 | 6 | C |
| ATOM | 752 | OG1 | THR | B | 95 | 30.049 | 20.307 | 15.622 | 1.00 | 33.37 | 8 | O |
| ATOM | 753 | CG2 | THR | B | 95 | 28.419 | 20.313 | 13.988 | 1.00 | 28.11 | 6 | C |
| ATOM | 754 | N | VAL | B | 96 | 27.282 | 17.062 | 15.626 | 1.00 | 31.67 | 7 | N |
| ATOM | 755 | CA | VAL | B | 96 | 26.280 | 16.125 | 15.237 | 1.00 | 31.65 | 6 | C |
| ATOM | 756 | C | VAL | B | 96 | 25.204 | 15.998 | 16.281 | 1.00 | 34.00 | 6 | C |
| ATOM | 757 | O | VAL | B | 96 | 23.990 | 16.068 | 15.900 | 1.00 | 34.23 | 8 | O |
| ATOM | 758 | CB | VAL | B | 96 | 26.847 | 14.753 | 14.807 | 1.00 | 31.85 | 6 | C |
| ATOM | 759 | CG1 | VAL | B | 96 | 25.644 | 13.870 | 14.537 | 1.00 | 26.93 | 6 | C |
| ATOM | 760 | CG2 | VAL | B | 96 | 27.728 | 14.870 | 13.600 | 1.00 | 30.75 | 6 | C |
| ATOM | 761 | N | GLN | B | 97 | 25.583 | 15.822 | 17.585 | 1.00 | 35.53 | 7 | N |
| ATOM | 762 | CA | GLN | B | 97 | 24.535 | 15.707 | 18.591 | 1.00 | 39.42 | 6 | C |
| ATOM | 763 | C | GLN | B | 97 | 23.508 | 16.833 | 18.568 | 1.00 | 40.87 | 6 | C |
| ATOM | 764 | O | GLN | B | 97 | 22.251 | 16.696 | 18.646 | 1.00 | 42.58 | 8 | O |
| ATOM | 765 | CB | GLN | B | 97 | 25.222 | 15.713 | 19.979 | 1.00 | 43.19 | 6 | C |
| ATOM | 766 | CG | GLN | B | 97 | 24.522 | 14.820 | 20.999 | 1.00 | 43.73 | 6 | C |
| ATOM | 767 | CD | GLN | B | 97 | 24.830 | 13.365 | 20.753 | 1.00 | 43.90 | 6 | C |
| ATOM | 768 | OE1 | GLN | B | 97 | 23.890 | 12.595 | 20.727 | 1.00 | 45.57 | 8 | O |
| ATOM | 769 | NE2 | GLN | B | 97 | 26.074 | 12.926 | 20.568 | 1.00 | 45.69 | 7 | N |
| ATOM | 770 | N | ARG | B | 98 | 24.031 | 18.072 | 18.413 | 1.00 | 39.48 | 7 | N |
| ATOM | 771 | CA | ARG | B | 98 | 23.128 | 19.226 | 18.440 | 1.00 | 40.24 | 6 | C |
| ATOM | 772 | C | ARG | B | 98 | 22.098 | 19.195 | 17.341 | 1.00 | 39.75 | 6 | C |
| ATOM | 773 | O | ARG | B | 98 | 21.002 | 19.709 | 17.489 | 1.00 | 40.11 | 8 | O |
| ATOM | 774 | CB | ARG | B | 98 | 23.952 | 20.478 | 18.429 | 1.00 | 44.31 | 6 | C |
| ATOM | 775 | CG | ARG | B | 98 | 23.194 | 21.802 | 18.364 | 1.00 | 47.62 | 6 | C |
| ATOM | 776 | CD | ARG | B | 98 | 24.299 | 22.864 | 18.409 | 1.00 | 51.78 | 6 | C |
| ATOM | 777 | NE | ARG | B | 98 | 25.365 | 22.690 | 19.401 | 1.00 | 56.13 | 7 | N |
| ATOM | 778 | CZ | ARG | B | 98 | 26.651 | 22.479 | 19.150 | 1.00 | 58.93 | 6 | C |
| ATOM | 779 | NH1 | ARG | B | 98 | 27.068 | 22.408 | 17.880 | 1.00 | 60.22 | 7 | N |
| ATOM | 780 | NH2 | ARG | B | 98 | 27.514 | 22.342 | 20.170 | 1.00 | 59.27 | 7 | N |
| ATOM | 781 | N | PHE | B | 99 | 22.384 | 18.624 | 16.172 | 1.00 | 36.80 | 7 | N |
| ATOM | 782 | CA | PHE | B | 99 | 21.448 | 18.496 | 15.062 | 1.00 | 32.26 | 6 | C |
| ATOM | 783 | C | PHE | B | 99 | 20.634 | 17.223 | 15.153 | 1.00 | 32.21 | 6 | C |
| ATOM | 784 | O | PHE | B | 99 | 19.543 | 17.051 | 14.611 | 1.00 | 26.79 | 8 | O |
| ATOM | 785 | CB | PHE | B | 99 | 22.228 | 18.509 | 13.751 | 1.00 | 32.53 | 6 | C |
| ATOM | 786 | CG | PHE | B | 99 | 22.757 | 19.814 | 13.242 | 1.00 | 32.58 | 6 | C |
| ATOM | 787 | CD1 | PHE | B | 99 | 24.099 | 20.146 | 13.375 | 1.00 | 33.27 | 6 | C |
| ATOM | 788 | CD2 | PHE | B | 99 | 21.918 | 20.720 | 12.623 | 1.00 | 31.75 | 6 | C |
| ATOM | 789 | CE1 | PHE | B | 99 | 24.591 | 21.364 | 12.900 | 1.00 | 30.86 | 6 | C |
| ATOM | 790 | CE2 | PHE | B | 99 | 22.383 | 21.911 | 12.151 | 1.00 | 31.14 | 6 | C |
| ATOM | 791 | CZ | PHE | B | 99 | 23.712 | 22.220 | 12.294 | 1.00 | 30.12 | 6 | C |
| ATOM | 792 | N | TYR | B | 100 | 21.179 | 16.174 | 15.830 | 1.00 | 32.52 | 7 | N |
| ATOM | 793 | CA | TYR | B | 100 | 20.407 | 14.956 | 16.039 | 1.00 | 34.89 | 6 | C |
| ATOM | 794 | C | TYR | B | 100 | 20.314 | 14.484 | 17.504 | 1.00 | 38.07 | 6 | C |
| ATOM | 795 | O | TYR | B | 100 | 20.820 | 13.421 | 17.940 | 1.00 | 39.72 | 8 | O |
| ATOM | 796 | CB | TYR | B | 100 | 21.116 | 13.906 | 15.223 | 1.00 | 34.13 | 6 | C |
| ATOM | 797 | CG | TYR | B | 100 | 21.204 | 14.102 | 13.753 | 1.00 | 32.10 | 6 | C |
| ATOM | 798 | CD1 | TYR | B | 100 | 22.161 | 14.880 | 13.136 | 1.00 | 30.26 | 6 | C |
| ATOM | 799 | CD2 | TYR | B | 100 | 20.246 | 13.446 | 12.972 | 1.00 | 31.08 | 6 | C |
| ATOM | 800 | CE1 | TYR | B | 100 | 22.174 | 15.009 | 11.772 | 1.00 | 31.79 | 6 | C |
| ATOM | 801 | CE2 | TYR | B | 100 | 20.271 | 13.582 | 11.610 | 1.00 | 31.02 | 6 | C |
| ATOM | 802 | CZ | TYR | B | 100 | 21.236 | 14.363 | 11.009 | 1.00 | 33.06 | 6 | C |
| ATOM | 803 | OH | TYR | B | 100 | 21.206 | 14.458 | 9.626 | 1.00 | 33.56 | 8 | O |
| ATOM | 804 | N | PRO | B | 101 | 19.691 | 15.256 | 18.375 | 1.00 | 39.52 | 7 | N |
| ATOM | 805 | CA | PRO | B | 101 | 19.523 | 14.889 | 19.759 | 1.00 | 39.48 | 6 | C |
| ATOM | 806 | C | PRO | B | 101 | 18.812 | 13.548 | 19.897 | 1.00 | 40.13 | 6 | C |
| ATOM | 807 | O | PRO | B | 101 | 17.784 | 13.279 | 19.269 | 1.00 | 40.64 | 8 | O |
| ATOM | 808 | CB | PRO | B | 101 | 18.777 | 16.048 | 20.360 | 1.00 | 41.05 | 6 | C |
| ATOM | 809 | CG | PRO | B | 101 | 18.127 | 16.807 | 19.236 | 1.00 | 41.11 | 6 | C |
| ATOM | 810 | CD | PRO | B | 101 | 18.961 | 16.530 | 18.012 | 1.00 | 40.28 | 6 | C |
| ATOM | 811 | N | GLY | B | 102 | 19.434 | 12.655 | 20.702 | 1.00 | 40.38 | 7 | N |
| ATOM | 812 | CA | GLY | B | 102 | 18.908 | 11.324 | 20.916 | 1.00 | 44.58 | 6 | C |
| ATOM | 813 | C | GLY | B | 102 | 18.792 | 10.520 | 19.619 | 1.00 | 47.15 | 6 | C |
| ATOM | 814 | O | GLY | B | 102 | 17.912 | 9.648 | 19.505 | 1.00 | 47.62 | 8 | O |
| ATOM | 815 | N | LYS | B | 103 | 19.600 | 10.794 | 18.568 | 1.00 | 45.68 | 7 | N |
| ATOM | 816 | CA | LYS | B | 103 | 19.549 | 10.132 | 17.288 | 1.00 | 43.82 | 6 | C |
| ATOM | 817 | C | LYS | B | 103 | 20.891 | 9.975 | 16.598 | 1.00 | 42.18 | 6 | C |
| ATOM | 818 | O | LYS | B | 103 | 20.938 | 10.057 | 15.383 | 1.00 | 39.86 | 8 | O |
| ATOM | 819 | CB | LYS | B | 103 | 18.464 | 10.777 | 16.414 | 1.00 | 44.11 | 6 | C |
| ATOM | 820 | CG | LYS | B | 103 | 17.115 | 10.111 | 16.771 | 1.00 | 46.78 | 6 | C |
| ATOM | 821 | CD | LYS | B | 103 | 16.056 | 10.719 | 15.871 | 1.00 | 50.69 | 6 | C |
| ATOM | 822 | CE | LYS | B | 103 | 14.672 | 10.284 | 16.385 | 1.00 | 52.78 | 6 | C |
| ATOM | 823 | NZ | LYS | B | 103 | 14.492 | 8.812 | 16.477 | 1.00 | 53.43 | 7 | N |
| ATOM | 824 | N | VAL | B | 104 | 21.952 | 9.725 | 17.338 | 1.00 | 41.60 | 7 | N |
| ATOM | 825 | CA | VAL | B | 104 | 23.333 | 9.524 | 16.989 | 1.00 | 41.58 | 6 | C |
| ATOM | 826 | C | VAL | B | 104 | 23.683 | 8.054 | 17.352 | 1.00 | 43.83 | 6 | C |
| ATOM | 827 | O | VAL | B | 104 | 23.445 | 7.552 | 18.444 | 1.00 | 43.31 | 8 | O |
| ATOM | 828 | CB | VAL | B | 104 | 24.383 | 10.384 | 17.709 | 1.00 | 40.91 | 6 | C |
| ATOM | 829 | CG1 | VAL | B | 104 | 25.786 | 10.161 | 17.176 | 1.00 | 39.66 | 6 | C |
| ATOM | 830 | CG2 | VAL | B | 104 | 24.017 | 11.853 | 17.699 | 1.00 | 39.95 | 6 | C |
| ATOM | 831 | N | GLU | B | 105 | 24.176 | 7.351 | 16.338 | 1.00 | 43.65 | 7 | N |
| ATOM | 832 | CA | GLU | B | 105 | 24.534 | 5.953 | 16.280 | 1.00 | 42.51 | 6 | C |
| ATOM | 833 | C | GLU | B | 105 | 26.015 | 5.860 | 15.994 | 1.00 | 41.76 | 6 | C |
| ATOM | 834 | O | GLU | B | 105 | 26.391 | 6.960 | 15.643 | 1.00 | 44.48 | 8 | O |
| ATOM | 835 | CB | GLU | B | 105 | 23.845 | 5.290 | 15.089 | 1.00 | 40.86 | 6 | C |
| ATOM | 836 | CG | GLU | B | 105 | 22.349 | 5.310 | 15.183 | 1.00 | 39.35 | 6 | C |
| ATOM | 837 | CD | GLU | B | 105 | 21.798 | 4.232 | 16.090 | 1.00 | 41.59 | 6 | C |
| ATOM | 838 | OE1 | GLU | B | 105 | 22.597 | 3.551 | 16.795 | 1.00 | 43.64 | 8 | O |
| ATOM | 839 | OE2 | GLU | B | 105 | 20.552 | 4.061 | 16.105 | 1.00 | 37.60 | 8 | O |
| ATOM | 840 | N | ARG | B | 106 | 26.724 | 4.797 | 16.175 | 1.00 | 40.20 | 7 | N |
| ATOM | 841 | CA | ARG | B | 106 | 28.137 | 4.733 | 15.923 | 1.00 | 42.67 | 6 | C |
| ATOM | 842 | C | ARG | B | 106 | 28.474 | 3.308 | 15.451 | 1.00 | 45.10 | 6 | C |
| ATOM | 843 | O | ARG | B | 106 | 27.714 | 2.376 | 15.650 | 1.00 | 45.19 | 8 | O |
| ATOM | 844 | CB | ARG | B | 106 | 28.931 | 5.146 | 17.138 | 1.00 | 43.37 | 6 | C |
| ATOM | 845 | CG | ARG | B | 106 | 28.446 | 4.552 | 18.444 | 1.00 | 46.18 | 6 | C |
| ATOM | 846 | CD | ARG | B | 106 | 29.143 | 5.365 | 19.555 | 1.00 | 48.07 | 6 | C |
| ATOM | 847 | NE | ARG | B | 106 | 28.239 | 6.492 | 19.822 | 1.00 | 51.33 | 7 | N |
| ATOM | 848 | CZ | ARG | B | 106 | 28.544 | 7.746 | 19.624 | 1.00 | 51.92 | 6 | C |
| ATOM | 849 | NH1 | ARG | B | 106 | 29.750 | 7.995 | 19.151 | 1.00 | 48.60 | 7 | N |
| ATOM | 850 | NH2 | ARG | B | 106 | 27.543 | 8.567 | 19.949 | 1.00 | 56.04 | 7 | N |
| ATOM | 851 | N | ILE | B | 107 | 29.576 | 3.141 | 14.751 | 1.00 | 43.69 | 7 | N |
| ATOM | 852 | CA | ILE | B | 107 | 30.129 | 1.931 | 14.233 | 1.00 | 41.71 | 6 | C |
| ATOM | 853 | C | ILE | B | 107 | 31.125 | 1.501 | 15.321 | 1.00 | 43.67 | 6 | C |
| ATOM | 854 | O | ILE | B | 107 | 31.848 | 2.407 | 15.791 | 1.00 | 43.55 | 8 | O |
| ATOM | 855 | CB | ILE | B | 107 | 30.872 | 2.229 | 12.921 | 1.00 | 39.20 | 6 | C |
| ATOM | 856 | CG1 | ILE | B | 107 | 29.916 | 2.751 | 11.880 | 1.00 | 34.88 | 6 | C |
| ATOM | 857 | CG2 | ILE | B | 107 | 31.682 | 1.007 | 12.454 | 1.00 | 38.36 | 6 | C |
| ATOM | 858 | CD1 | ILE | B | 107 | 28.907 | 1.922 | 11.238 | 1.00 | 33.24 | 6 | C |
| ATOM | 859 | N | GLU | B | 108 | 31.167 | 0.235 | 15.725 | 1.00 | 45.03 | 7 | N |
| ATOM | 860 | CA | GLU | B | 108 | 32.041 | −0.219 | 16.805 | 1.00 | 43.40 | 6 | C |
| ATOM | 861 | C | GLU | B | 108 | 33.157 | −1.081 | 16.281 | 1.00 | 44.95 | 6 | C |
| ATOM | 862 | O | GLU | B | 108 | 32.985 | −1.716 | 15.268 | 1.00 | 46.74 | 8 | O |
| ATOM | 863 | CB | GLU | B | 108 | 31.330 | −0.762 | 17.947 | 0.00 | 25.00 | 6 | C |
| ATOM | 864 | CG | GLU | B | 108 | 30.185 | 0.121 | 18.410 | 0.00 | 25.00 | 6 | C |
| ATOM | 865 | CD | GLU | B | 108 | 29.338 | −0.627 | 19.419 | 0.00 | 25.00 | 6 | C |
| ATOM | 866 | OE1 | GLU | B | 108 | 29.478 | −1.864 | 19.515 | 0.00 | 25.00 | 8 | O |
| ATOM | 867 | OE2 | GLU | B | 108 | 28.531 | 0.025 | 20.117 | 0.00 | 25.00 | 8 | O |
| ATOM | 868 | N | ALA | B | 109 | 34.275 | −1.131 | 16.962 | 1.00 | 45.26 | 7 | N |
| ATOM | 869 | CA | ALA | B | 109 | 35.403 | −2.010 | 16.651 | 1.00 | 46.19 | 6 | C |
| ATOM | 870 | C | ALA | B | 109 | 34.725 | −3.371 | 16.549 | 1.00 | 44.23 | 6 | C |
| ATOM | 871 | O | ALA | B | 109 | 33.738 | −3.603 | 17.277 | 1.00 | 49.73 | 8 | O |
| ATOM | 872 | CB | ALA | B | 109 | 36.445 | −1.881 | 17.773 | 1.00 | 43.79 | 6 | C |
| ATOM | 873 | N | PRO | B | 110 | 35.147 | −4.201 | 15.637 | 1.00 | 40.81 | 7 | N |
| ATOM | 874 | CA | PRO | B | 110 | 36.277 | −4.023 | 14.743 | 1.00 | 39.15 | 6 | C |
| ATOM | 875 | C | PRO | B | 110 | 35.860 | −3.574 | 13.354 | 1.00 | 39.84 | 6 | C |
| ATOM | 876 | O | PRO | B | 110 | 36.490 | −3.601 | 12.271 | 1.00 | 37.25 | 8 | O |
| ATOM | 877 | CB | PRO | B | 110 | 36.819 | −5.469 | 14.566 | 1.00 | 35.24 | 6 | C |
| ATOM | 878 | CG | PRO | B | 110 | 35.528 | −6.169 | 14.376 | 1.00 | 36.94 | 6 | C |
| ATOM | 879 | CD | PRO | B | 110 | 34.554 | −5.571 | 15.402 | 1.00 | 40.51 | 6 | C |
| ATOM | 880 | N | GLY | B | 111 | 34.566 | −3.152 | 13.380 | 1.00 | 40.10 | 7 | N |
| ATOM | 881 | CA | GLY | B | 111 | 33.890 | −2.626 | 12.209 | 1.00 | 40.74 | 6 | C |
| ATOM | 882 | C | GLY | B | 111 | 34.559 | −1.264 | 11.868 | 1.00 | 42.01 | 6 | C |
| ATOM | 883 | O | GLY | B | 111 | 34.876 | −0.490 | 12.738 | 1.00 | 39.53 | 8 | O |
| ATOM | 884 | N | THR | B | 112 | 34.764 | −1.022 | 10.584 | 1.00 | 41.30 | 7 | N |
| ATOM | 885 | CA | THR | B | 112 | 35.395 | 0.188 | 10.060 | 1.00 | 40.36 | 6 | C |
| ATOM | 886 | C | THR | B | 112 | 34.541 | 0.737 | 8.934 | 1.00 | 39.71 | 6 | C |
| ATOM | 887 | O | THR | B | 112 | 33.941 | −0.101 | 8.227 | 1.00 | 39.57 | 8 | O |
| ATOM | 888 | CB | THR | B | 112 | 36.777 | −0.322 | 9.671 | 1.00 | 40.12 | 6 | C |
| ATOM | 889 | OG1 | THR | B | 112 | 37.807 | 0.417 | 10.296 | 1.00 | 44.76 | 8 | O |
| ATOM | 890 | CG2 | THR | B | 112 | 36.967 | −0.121 | 8.195 | 1.00 | 35.62 | 6 | C |
| ATOM | 891 | N | VAL | B | 113 | 34.381 | 2.025 | 8.732 | 1.00 | 37.76 | 7 | N |
| ATOM | 892 | CA | VAL | B | 113 | 33.585 | 2.724 | 7.743 | 1.00 | 36.05 | 6 | C |
| ATOM | 893 | C | VAL | B | 113 | 33.980 | 4.228 | 7.620 | 1.00 | 38.06 | 6 | C |
| ATOM | 894 | O | VAL | B | 113 | 34.192 | 5.017 | 8.542 | 1.00 | 37.06 | 8 | O |
| ATOM | 895 | CB | VAL | B | 113 | 32.077 | 2.861 | 8.066 | 1.00 | 35.13 | 6 | C |
| ATOM | 896 | CG1 | VAL | B | 113 | 31.241 | 3.512 | 6.927 | 1.00 | 32.91 | 6 | C |
| ATOM | 897 | CG2 | VAL | B | 113 | 31.253 | 1.637 | 8.470 | 1.00 | 29.05 | 6 | C |
| ATOM | 898 | N | GLU | B | 114 | 34.069 | 4.700 | 6.366 | 1.00 | 37.61 | 7 | N |
| ATOM | 899 | CA | GLU | B | 114 | 34.367 | 6.080 | 5.991 | 1.00 | 33.55 | 6 | C |
| ATOM | 900 | C | GLU | B | 114 | 33.205 | 6.403 | 5.061 | 1.00 | 33.96 | 6 | C |
| ATOM | 901 | O | GLU | B | 114 | 32.951 | 5.804 | 4.002 | 1.00 | 32.65 | 8 | O |
| ATOM | 902 | CB | GLU | B | 114 | 35.657 | 6.179 | 5.242 | 1.00 | 35.94 | 6 | C |
| ATOM | 903 | CG | GLU | B | 114 | 36.826 | 5.855 | 6.180 | 1.00 | 32.47 | 6 | C |
| ATOM | 904 | CD | GLU | B | 114 | 38.177 | 6.300 | 5.818 | 1.00 | 30.93 | 6 | C |
| ATOM | 905 | OE1 | GLU | B | 114 | 38.471 | 6.837 | 4.713 | 1.00 | 29.08 | 8 | O |
| ATOM | 906 | OE2 | GLU | B | 114 | 38.944 | 6.038 | 6.823 | 1.00 | 36.30 | 8 | O |
| ATOM | 907 | N | ALA | B | 115 | 32.354 | 7.362 | 5.499 | 1.00 | 30.75 | 7 | N |
| ATOM | 908 | CA | ALA | B | 115 | 31.189 | 7.770 | 4.760 | 1.00 | 27.99 | 6 | C |
| ATOM | 909 | C | ALA | B | 115 | 31.472 | 8.266 | 3.352 | 1.00 | 28.85 | 6 | C |
| ATOM | 910 | O | ALA | B | 115 | 30.528 | 8.306 | 2.549 | 1.00 | 30.04 | 8 | O |
| ATOM | 911 | CB | ALA | B | 115 | 30.335 | 8.520 | 5.682 | 1.00 | 23.95 | 6 | C |
| ATOM | 912 | N | GLY | B | 116 | 32.668 | 8.536 | 2.859 | 1.00 | 29.35 | 7 | N |
| ATOM | 913 | CA | GLY | B | 116 | 32.866 | 8.859 | 1.460 | 1.00 | 31.95 | 6 | C |
| ATOM | 914 | C | GLY | B | 116 | 32.685 | 7.585 | 0.592 | 1.00 | 33.73 | 6 | C |
| ATOM | 915 | O | GLY | B | 116 | 32.609 | 7.635 | −0.626 | 1.00 | 32.12 | 8 | O |
| ATOM | 916 | N | ASP | B | 117 | 32.609 | 6.404 | 1.238 | 1.00 | 34.95 | 7 | N |
| ATOM | 917 | CA | ASP | B | 117 | 32.320 | 5.158 | 0.497 | 1.00 | 35.94 | 6 | C |
| ATOM | 918 | C | ASP | B | 117 | 30.794 | 5.020 | 0.404 | 1.00 | 37.55 | 6 | C |
| ATOM | 919 | O | ASP | B | 117 | 30.365 | 4.039 | −0.168 | 1.00 | 39.48 | 8 | O |
| ATOM | 920 | CB | ASP | B | 117 | 32.773 | 3.840 | 1.153 | 1.00 | 32.97 | 6 | C |
| ATOM | 921 | CG | ASP | B | 117 | 34.291 | 3.737 | 0.961 | 1.00 | 35.32 | 6 | C |
| ATOM | 922 | OD1 | ASP | B | 117 | 34.648 | 3.967 | −0.227 | 1.00 | 37.24 | 8 | O |
| ATOM | 923 | OD2 | ASP | B | 117 | 35.119 | 3.460 | 1.857 | 1.00 | 33.30 | 8 | O |
| ATOM | 924 | N | ILE | B | 118 | 29.958 | 5.918 | 0.894 | 1.00 | 36.88 | 7 | N |
| ATOM | 925 | CA | ILE | B | 118 | 28.535 | 5.725 | 0.878 | 1.00 | 38.60 | 6 | C |
| ATOM | 926 | C | ILE | B | 118 | 27.775 | 6.695 | 0.023 | 1.00 | 39.93 | 6 | C |
| ATOM | 927 | O | ILE | B | 118 | 27.946 | 7.885 | 0.173 | 1.00 | 39.34 | 8 | O |
| ATOM | 928 | CB | ILE | B | 118 | 28.087 | 5.901 | 2.381 | 1.00 | 39.31 | 6 | C |
| ATOM | 929 | CG1 | ILE | B | 118 | 28.725 | 4.876 | 3.331 | 1.00 | 39.76 | 6 | C |
| ATOM | 930 | CG2 | ILE | B | 118 | 26.591 | 5.769 | 2.480 | 1.00 | 35.77 | 6 | C |
| ATOM | 931 | CD1 | ILE | B | 118 | 28.380 | 5.090 | 4.813 | 1.00 | 40.77 | 6 | C |
| ATOM | 932 | N | MET | B | 119 | 26.924 | 6.317 | −0.908 | 1.00 | 40.56 | 7 | N |
| ATOM | 933 | CA | MET | B | 119 | 26.172 | 7.155 | −1.812 | 1.00 | 36.86 | 6 | C |
| ATOM | 934 | C | MET | B | 119 | 24.673 | 7.079 | −1.488 | 1.00 | 38.06 | 6 | C |
| ATOM | 935 | O | MET | B | 119 | 24.066 | 6.002 | −1.604 | 1.00 | 40.53 | 8 | O |
| ATOM | 936 | CB | MET | B | 119 | 26.271 | 6.611 | −3.236 | 1.00 | 32.64 | 6 | C |
| ATOM | 937 | CG | MET | B | 119 | 25.582 | 7.617 | −4.186 | 1.00 | 33.59 | 6 | C |
| ATOM | 938 | SD | MET | B | 119 | 25.930 | 7.493 | −5.943 | 1.00 | 29.84 | 16 | S |
| ATOM | 939 | CE | MET | B | 119 | 25.211 | 5.840 | −6.168 | 1.00 | 30.68 | 6 | C |
| ATOM | 940 | N | MET | B | 120 | 24.075 | 8.166 | −1.129 | 1.00 | 35.23 | 7 | N |
| ATOM | 941 | CA | MET | B | 120 | 22.701 | 8.282 | −0.789 | 1.00 | 36.36 | 6 | C |
| ATOM | 942 | C | MET | B | 120 | 21.793 | 8.443 | −2.026 | 1.00 | 37.09 | 6 | C |
| ATOM | 943 | O | MET | B | 120 | 21.962 | 9.385 | −2.798 | 1.00 | 35.44 | 8 | O |
| ATOM | 944 | CB | MET | B | 120 | 22.441 | 9.517 | 0.143 | 1.00 | 31.36 | 6 | C |
| ATOM | 945 | CG | MET | B | 120 | 21.022 | 9.526 | 0.646 | 1.00 | 22.26 | 6 | C |
| ATOM | 946 | SD | MET | B | 120 | 20.634 | 10.869 | 1.794 | 1.00 | 22.70 | 16 | S |
| ATOM | 947 | CE | MET | B | 120 | 20.868 | 12.229 | 0.616 | 1.00 | 15.45 | 6 | C |
| ATOM | 948 | N | VAL | B | 121 | 20.805 | 7.569 | −2.191 | 1.00 | 36.03 | 7 | N |
| ATOM | 949 | CA | VAL | B | 121 | 19.870 | 7.646 | −3.295 | 1.00 | 37.51 | 6 | C |
| ATOM | 950 | C | VAL | B | 121 | 18.474 | 7.472 | −2.655 | 1.00 | 40.29 | 6 | C |
| ATOM | 951 | O | VAL | B | 121 | 18.029 | 6.412 | −2.243 | 1.00 | 42.42 | 8 | O |
| ATOM | 952 | CB | VAL | B | 121 | 19.998 | 6.666 | −4.461 | 1.00 | 35.14 | 6 | C |
| ATOM | 953 | CG1 | VAL | B | 121 | 19.023 | 6.984 | −5.582 | 1.00 | 31.14 | 6 | C |
| ATOM | 954 | CG2 | VAL | B | 121 | 21.382 | 6.709 | −5.107 | 1.00 | 35.61 | 6 | C |
| ATOM | 955 | N | GLY | B | 122 | 17.767 | 8.586 | −2.561 | 1.00 | 39.89 | 7 | N |
| ATOM | 956 | CA | GLY | B | 122 | 16.481 | 8.618 | −1.949 | 1.00 | 39.44 | 6 | C |
| ATOM | 957 | C | GLY | B | 122 | 16.700 | 8.032 | −0.540 | 1.00 | 42.45 | 6 | C |
| ATOM | 958 | O | GLY | B | 122 | 17.363 | 8.639 | 0.293 | 1.00 | 39.63 | 8 | O |
| ATOM | 959 | N | ASP | B | 123 | 16.173 | 6.833 | −0.224 | 1.00 | 45.45 | 7 | N |
| ATOM | 960 | CA | ASP | B | 123 | 16.352 | 6.323 | 1.123 | 1.00 | 45.21 | 6 | C |
| ATOM | 961 | C | ASP | B | 123 | 17.237 | 5.076 | 1.093 | 1.00 | 43.17 | 6 | C |
| ATOM | 962 | O | ASP | B | 123 | 17.363 | 4.454 | 2.157 | 1.00 | 41.69 | 8 | O |
| ATOM | 963 | CB | ASP | B | 123 | 15.132 | 6.149 | 1.947 | 1.00 | 48.52 | 6 | C |
| ATOM | 964 | CG | ASP | B | 123 | 14.499 | 7.291 | 2.660 | 1.00 | 52.47 | 6 | C |
| ATOM | 965 | OD1 | ASP | B | 123 | 14.796 | 8.449 | 3.001 | 1.00 | 54.40 | 8 | O |
| ATOM | 966 | OD2 | ASP | B | 123 | 13.320 | 7.050 | 3.045 | 1.00 | 57.91 | 8 | O |
| ATOM | 967 | N | HIS | B | 124 | 17.859 | 4.926 | −0.068 | 1.00 | 40.33 | 7 | N |
| ATOM | 968 | CA | HIS | B | 124 | 18.764 | 3.788 | −0.169 | 1.00 | 41.77 | 6 | C |
| ATOM | 969 | C | HIS | B | 124 | 20.184 | 4.327 | −0.075 | 1.00 | 41.51 | 6 | C |
| ATOM | 970 | O | HIS | B | 124 | 20.474 | 5.384 | −0.539 | 1.00 | 39.70 | 8 | O |
| ATOM | 971 | CB | HIS | B | 124 | 18.443 | 2.915 | −1.411 | 1.00 | 39.31 | 6 | C |
| ATOM | 972 | CG | HIS | B | 124 | 19.327 | 1.707 | −1.387 | 1.00 | 40.46 | 6 | C |
| ATOM | 973 | ND1 | HIS | B | 124 | 19.014 | 0.669 | −0.549 | 1.00 | 40.16 | 7 | N |
| ATOM | 974 | CD2 | HIS | B | 124 | 20.464 | 1.341 | −2.035 | 1.00 | 40.50 | 6 | C |
| ATOM | 975 | CE1 | HIS | B | 124 | 19.910 | −0.301 | −0.671 | 1.00 | 39.18 | 6 | C |
| ATOM | 976 | NE2 | HIS | B | 124 | 20.793 | 0.081 | −1.563 | 1.00 | 40.33 | 7 | N |
| ATOM | 977 | N | PHE | B | 125 | 21.054 | 3.596 | 0.613 | 1.00 | 41.51 | 7 | N |
| ATOM | 978 | CA | PHE | B | 125 | 22.433 | 3.890 | 0.783 | 1.00 | 40.24 | 6 | C |
| ATOM | 979 | C | PHE | B | 125 | 23.316 | 2.758 | 0.175 | 1.00 | 40.86 | 6 | C |
| ATOM | 980 | O | PHE | B | 125 | 23.375 | 1.667 | 0.724 | 1.00 | 41.27 | 8 | O |
| ATOM | 981 | CB | PHE | B | 125 | 22.846 | 4.130 | 2.252 | 1.00 | 37.30 | 6 | C |
| ATOM | 982 | CG | PHE | B | 125 | 22.111 | 5.345 | 2.786 | 1.00 | 36.19 | 6 | C |
| ATOM | 983 | CD1 | PHE | B | 125 | 20.765 | 5.232 | 3.079 | 1.00 | 34.48 | 6 | C |
| ATOM | 984 | CD2 | PHE | B | 125 | 22.738 | 6.570 | 2.991 | 1.00 | 32.25 | 6 | C |
| ATOM | 985 | CE1 | PHE | B | 125 | 20.042 | 6.313 | 3.562 | 1.00 | 34.75 | 6 | C |
| ATOM | 986 | CE2 | PHE | B | 125 | 21.964 | 7.584 | 3.474 | 1.00 | 33.63 | 6 | C |
| ATOM | 987 | CZ | PHE | B | 125 | 20.634 | 7.519 | 3.774 | 1.00 | 31.68 | 6 | C |
| ATOM | 988 | N | TYR | B | 126 | 24.029 | 3.062 | −0.915 | 1.00 | 37.60 | 7 | N |
| ATOM | 989 | CA | TYR | B | 126 | 24.974 | 2.185 | −1.534 | 1.00 | 38.23 | 6 | C |
| ATOM | 990 | C | TYR | B | 126 | 26.273 | 2.294 | −0.794 | 1.00 | 40.31 | 6 | C |
| ATOM | 991 | O | TYR | B | 126 | 26.700 | 3.408 | −0.547 | 1.00 | 42.77 | 8 | O |
| ATOM | 992 | CB | TYR | B | 126 | 25.165 | 2.604 | −2.979 | 1.00 | 38.88 | 6 | C |
| ATOM | 993 | CG | TYR | B | 126 | 23.983 | 2.322 | −3.855 | 1.00 | 36.06 | 6 | C |
| ATOM | 994 | CD1 | TYR | B | 126 | 23.209 | 3.359 | −4.287 | 1.00 | 36.47 | 6 | C |
| ATOM | 995 | CD2 | TYR | B | 126 | 23.629 | 1.032 | −4.252 | 1.00 | 39.47 | 6 | C |
| ATOM | 996 | CE1 | TYR | B | 126 | 22.120 | 3.115 | −5.090 | 1.00 | 38.21 | 6 | C |
| ATOM | 997 | CE2 | TYR | B | 126 | 22.513 | 0.768 | −5.076 | 1.00 | 38.32 | 6 | C |
| ATOM | 998 | CZ | TYR | B | 126 | 21.777 | 1.832 | −5.482 | 1.00 | 37.26 | 6 | C |
| ATOM | 999 | OH | TYR | B | 126 | 20.684 | 1.725 | −6.273 | 1.00 | 39.55 | 8 | O |
| ATOM | 1000 | N | ILE | B | 127 | 26.901 | 1.211 | −0.329 | 1.00 | 43.25 | 7 | N |
| ATOM | 1001 | CA | ILE | B | 127 | 28.132 | 1.298 | 0.482 | 1.00 | 40.57 | 6 | C |
| ATOM | 1002 | C | ILE | B | 127 | 29.281 | 0.562 | −0.131 | 1.00 | 43.11 | 6 | C |
| ATOM | 1003 | O | ILE | B | 127 | 29.193 | −0.667 | −0.228 | 1.00 | 49.30 | 8 | O |
| ATOM | 1004 | CB | ILE | B | 127 | 27.847 | 0.665 | 1.851 | 1.00 | 34.65 | 6 | C |
| ATOM | 1005 | CG1 | ILE | B | 127 | 26.605 | 1.269 | 2.513 | 1.00 | 34.17 | 6 | C |
| ATOM | 1006 | CG2 | ILE | B | 127 | 29.004 | 0.749 | 2.779 | 1.00 | 33.07 | 6 | C |
| ATOM | 1007 | CD1 | ILE | B | 127 | 26.176 | 0.538 | 3.789 | 1.00 | 27.95 | 6 | C |
| ATOM | 1008 | N | GLY | B | 128 | 30.371 | 1.107 | −0.581 | 1.00 | 42.78 | 7 | N |
| ATOM | 1009 | CA | GLY | B | 128 | 31.448 | 0.336 | −1.179 | 1.00 | 43.25 | 6 | C |
| ATOM | 1010 | C | GLY | B | 128 | 32.309 | −0.370 | −0.183 | 1.00 | 45.46 | 6 | C |
| ATOM | 1011 | O | GLY | B | 128 | 32.552 | 0.155 | 0.892 | 1.00 | 47.44 | 8 | O |
| ATOM | 1012 | N | GLU | B | 129 | 32.778 | −1.582 | −0.511 | 1.00 | 48.28 | 7 | N |
| ATOM | 1013 | CA | GLU | B | 129 | 33.649 | −2.259 | 0.467 | 1.00 | 48.01 | 6 | C |
| ATOM | 1014 | C | GLU | B | 129 | 35.019 | −1.761 | 0.012 | 1.00 | 45.40 | 6 | C |
| ATOM | 1015 | O | GLU | B | 129 | 35.102 | −1.714 | −1.205 | 1.00 | 42.64 | 8 | O |
| ATOM | 1016 | CB | GLU | B | 129 | 33.323 | −3.708 | 0.438 | 1.00 | 51.12 | 6 | C |
| ATOM | 1017 | CG | GLU | B | 129 | 34.143 | −4.434 | 1.495 | 1.00 | 56.38 | 6 | C |
| ATOM | 1018 | CD | GLU | B | 129 | 33.924 | −5.944 | 1.442 | 1.00 | 61.81 | 6 | C |
| ATOM | 1019 | OE1 | GLU | B | 129 | 33.062 | −6.392 | 0.620 | 1.00 | 62.96 | 8 | O |
| ATOM | 1020 | OE2 | GLU | B | 129 | 34.665 | −6.568 | 2.259 | 1.00 | 62.20 | 8 | O |
| ATOM | 1021 | N | SER | B | 130 | 35.936 | −1.454 | 0.932 | 1.00 | 42.28 | 7 | N |
| ATOM | 1022 | CA | SER | B | 130 | 37.180 | −0.910 | 0.382 | 1.00 | 43.30 | 6 | C |
| ATOM | 1023 | C | SER | B | 130 | 38.299 | −1.017 | 1.355 | 1.00 | 42.55 | 6 | C |
| ATOM | 1024 | O | SER | B | 130 | 38.074 | −1.560 | 2.403 | 1.00 | 43.03 | 8 | O |
| ATOM | 1025 | CB | SER | B | 130 | 36.874 | 0.600 | 0.122 | 1.00 | 44.95 | 6 | C |
| ATOM | 1026 | OG | SER | B | 130 | 36.588 | 1.247 | 1.391 | 1.00 | 41.31 | 8 | O |
| ATOM | 1027 | N | ALA | B | 131 | 39.451 | −0.432 | 1.034 | 1.00 | 45.01 | 7 | N |
| ATOM | 1028 | CA | ALA | B | 131 | 40.512 | −0.539 | 2.073 | 1.00 | 47.73 | 6 | C |
| ATOM | 1029 | C | ALA | B | 131 | 39.916 | 0.069 | 3.327 | 1.00 | 47.49 | 6 | C |
| ATOM | 1030 | O | ALA | B | 131 | 39.947 | −0.497 | 4.394 | 1.00 | 51.26 | 8 | O |
| ATOM | 1031 | CB | ALA | B | 131 | 41.604 | 0.389 | 1.573 | 1.00 | 52.68 | 6 | C |
| ATOM | 1032 | N | ARG | B | 132 | 39.286 | 1.246 | 3.204 | 1.00 | 45.50 | 7 | N |
| ATOM | 1033 | CA | ARG | B | 132 | 38.766 | 1.944 | 4.374 | 1.00 | 42.43 | 6 | C |
| ATOM | 1034 | C | ARG | B | 132 | 37.438 | 1.490 | 4.865 | 1.00 | 40.73 | 6 | C |
| ATOM | 1035 | O | ARG | B | 132 | 37.166 | 1.806 | 6.048 | 1.00 | 40.78 | 8 | O |
| ATOM | 1036 | CB | ARG | B | 132 | 39.078 | 3.458 | 4.131 | 1.00 | 40.48 | 6 | C |
| ATOM | 1037 | CG | ARG | B | 132 | 40.029 | 4.269 | 3.563 | 0.00 | 25.00 | 6 | C |
| ATOM | 1038 | CD | ARG | B | 132 | 41.110 | 4.444 | 4.592 | 0.00 | 25.00 | 6 | C |
| ATOM | 1039 | NE | ARG | B | 132 | 42.264 | 5.181 | 4.075 | 0.00 | 25.00 | 7 | N |
| ATOM | 1040 | CZ | ARG | B | 132 | 42.376 | 6.494 | 3.868 | 0.00 | 25.00 | 6 | C |
| ATOM | 1041 | NH1 | ARG | B | 132 | 41.376 | 7.360 | 4.124 | 0.00 | 25.00 | 7 | N |
| ATOM | 1042 | NH2 | ARG | B | 132 | 43.535 | 6.959 | 3.383 | 0.00 | 25.00 | 7 | N |
| ATOM | 1043 | N | THR | B | 133 | 36.545 | 0.811 | 4.201 | 1.00 | 39.30 | 7 | N |
| ATOM | 1044 | CA | THR | B | 133 | 35.272 | 0.429 | 4.817 | 1.00 | 40.95 | 6 | C |
| ATOM | 1045 | C | THR | B | 133 | 35.168 | −1.115 | 4.794 | 1.00 | 41.88 | 6 | C |
| ATOM | 1046 | O | THR | B | 133 | 34.858 | −1.732 | 3.793 | 1.00 | 40.26 | 8 | O |
| ATOM | 1047 | CB | THR | B | 133 | 34.121 | 1.098 | 4.070 | 1.00 | 40.96 | 6 | C |
| ATOM | 1048 | OG1 | THR | B | 133 | 34.054 | 2.552 | 4.216 | 1.00 | 42.16 | 8 | O |
| ATOM | 1049 | CG2 | THR | B | 133 | 32.756 | 0.516 | 4.424 | 1.00 | 36.15 | 6 | C |
| ATOM | 1050 | N | ASN | B | 134 | 35.445 | −1.774 | 5.929 | 1.00 | 41.65 | 7 | N |
| ATOM | 1051 | CA | ASN | B | 134 | 35.436 | −3.241 | 5.996 | 1.00 | 39.37 | 6 | C |
| ATOM | 1052 | C | ASN | B | 134 | 34.093 | −3.886 | 5.927 | 1.00 | 39.34 | 6 | C |
| ATOM | 1053 | O | ASN | B | 134 | 33.069 | −3.220 | 6.081 | 1.00 | 41.85 | 8 | O |
| ATOM | 1054 | CB | ASN | B | 134 | 36.341 | −3.594 | 7.168 | 1.00 | 34.76 | 6 | C |
| ATOM | 1055 | CG | ASN | B | 134 | 35.829 | −3.484 | 8.533 | 1.00 | 35.67 | 6 | C |
| ATOM | 1056 | OD1 | ASN | B | 134 | 34.584 | −3.460 | 8.689 | 1.00 | 33.58 | 8 | O |
| ATOM | 1057 | ND2 | ASN | B | 134 | 36.821 | −3.432 | 9.477 | 1.00 | 36.21 | 7 | N |
| ATOM | 1058 | N | ALA | B | 135 | 33.924 | −5.183 | 5.718 | 1.00 | 39.70 | 7 | N |
| ATOM | 1059 | CA | ALA | B | 135 | 32.622 | −5.856 | 5.687 | 1.00 | 40.46 | 6 | C |
| ATOM | 1060 | C | ALA | B | 135 | 31.911 | −5.763 | 7.023 | 1.00 | 40.58 | 6 | C |
| ATOM | 1061 | O | ALA | B | 135 | 30.686 | −5.560 | 7.142 | 1.00 | 40.83 | 8 | O |
| ATOM | 1062 | CB | ALA | B | 135 | 32.744 | −7.328 | 5.258 | 1.00 | 42.50 | 6 | C |
| ATOM | 1063 | N | GLU | B | 136 | 32.645 | −5.811 | 8.141 | 1.00 | 41.50 | 7 | N |
| ATOM | 1064 | CA | GLU | B | 136 | 31.938 | −5.653 | 9.426 | 1.00 | 41.21 | 6 | C |
| ATOM | 1065 | C | GLU | B | 136 | 31.286 | −4.262 | 9.455 | 1.00 | 43.76 | 6 | C |
| ATOM | 1066 | O | GLU | B | 136 | 30.140 | −3.916 | 9.837 | 1.00 | 44.13 | 8 | O |
| ATOM | 1067 | CB | GLU | B | 136 | 32.965 | −5.957 | 10.516 | 1.00 | 43.15 | 6 | C |
| ATOM | 1068 | CG | GLU | B | 136 | 32.520 | −5.653 | 11.938 | 1.00 | 46.87 | 6 | C |
| ATOM | 1069 | CD | GLU | B | 136 | 31.174 | −6.340 | 12.158 | 1.00 | 48.23 | 6 | C |
| ATOM | 1070 | OE1 | GLU | B | 136 | 30.915 | −7.296 | 11.347 | 1.00 | 50.53 | 8 | O |
| ATOM | 1071 | OE2 | GLU | B | 136 | 30.486 | −5.906 | 13.085 | 1.00 | 47.90 | 8 | O |
| ATOM | 1072 | N | GLY | B | 137 | 32.058 | −3.261 | 8.975 | 1.00 | 43.51 | 7 | N |
| ATOM | 1073 | CA | GLY | B | 137 | 31.651 | −1.860 | 8.948 | 1.00 | 41.79 | 6 | C |
| ATOM | 1074 | C | GLY | B | 137 | 30.320 | −1.749 | 8.202 | 1.00 | 41.07 | 6 | C |
| ATOM | 1075 | O | GLY | B | 137 | 29.281 | −1.284 | 8.682 | 1.00 | 42.20 | 8 | O |
| ATOM | 1076 | N | ALA | B | 138 | 30.366 | −2.188 | 6.945 | 1.00 | 38.21 | 7 | N |
| ATOM | 1077 | CA | ALA | B | 138 | 29.198 | −2.165 | 6.089 | 1.00 | 37.54 | 6 | C |
| ATOM | 1078 | C | ALA | B | 138 | 28.023 | −2.869 | 6.749 | 1.00 | 40.46 | 6 | C |
| ATOM | 1079 | O | ALA | B | 138 | 26.918 | −2.385 | 6.673 | 1.00 | 40.61 | 8 | O |
| ATOM | 1080 | CB | ALA | B | 138 | 29.547 | −2.908 | 4.809 | 1.00 | 35.18 | 6 | C |
| ATOM | 1081 | N | ARG | B | 139 | 28.282 | −4.065 | 7.341 | 1.00 | 41.57 | 7 | N |
| ATOM | 1082 | CA | ARG | B | 139 | 27.246 | −4.808 | 8.005 | 1.00 | 43.16 | 6 | C |
| ATOM | 1083 | C | ARG | B | 139 | 26.694 | −3.985 | 9.162 | 1.00 | 42.14 | 6 | C |
| ATOM | 1084 | O | ARG | B | 139 | 25.460 | −3.815 | 9.152 | 1.00 | 44.47 | 8 | O |
| ATOM | 1085 | CB | ARG | B | 139 | 27.720 | −6.202 | 8.507 | 1.00 | 43.18 | 6 | C |
| ATOM | 1086 | CG | ARG | B | 139 | 27.150 | −7.131 | 9.361 | 0.00 | 25.00 | 6 | C |
| ATOM | 1087 | CD | ARG | B | 139 | 27.860 | −8.093 | 10.302 | 0.00 | 25.00 | 6 | C |
| ATOM | 1088 | NE | ARG | B | 139 | 28.382 | −7.409 | 11.482 | 0.00 | 25.00 | 7 | N |
| ATOM | 1089 | CZ | ARG | B | 139 | 27.615 | −6.973 | 12.472 | 0.00 | 25.00 | 6 | C |
| ATOM | 1090 | NH1 | ARG | B | 139 | 26.304 | −7.162 | 12.411 | 0.00 | 25.00 | 7 | N |
| ATOM | 1091 | NH2 | ARG | B | 139 | 28.149 | −6.351 | 13.512 | 0.00 | 25.00 | 7 | N |
| ATOM | 1092 | N | GLN | B | 140 | 27.501 | −3.480 | 10.086 | 1.00 | 39.52 | 7 | N |
| ATOM | 1093 | CA | GLN | B | 140 | 26.805 | −2.709 | 11.160 | 1.00 | 39.49 | 6 | C |
| ATOM | 1094 | C | GLN | B | 140 | 26.096 | −1.547 | 10.486 | 1.00 | 40.72 | 6 | C |
| ATOM | 1095 | O | GLN | B | 140 | 24.979 | −1.247 | 10.925 | 1.00 | 45.21 | 8 | O |
| ATOM | 1096 | CB | GLN | B | 140 | 27.638 | −2.238 | 12.333 | 1.00 | 36.81 | 6 | C |
| ATOM | 1097 | CG | GLN | B | 140 | 28.528 | −3.125 | 13.168 | 1.00 | 33.96 | 6 | C |
| ATOM | 1098 | CD | GLN | B | 140 | 29.659 | −2.363 | 13.790 | 1.00 | 37.91 | 6 | C |
| ATOM | 1099 | OE1 | GLN | B | 140 | 29.326 | −1.177 | 14.119 | 1.00 | 43.19 | 8 | O |
| ATOM | 1100 | NE2 | GLN | B | 140 | 30.900 | −2.791 | 14.013 | 1.00 | 33.63 | 7 | N |
| ATOM | 1101 | N | MET | B | 141 | 26.640 | −0.878 | 9.467 | 1.00 | 41.05 | 7 | N |
| ATOM | 1102 | CA | MET | B | 141 | 26.014 | 0.281 | 8.816 | 1.00 | 39.36 | 6 | C |
| ATOM | 1103 | C | MET | B | 141 | 24.664 | −0.076 | 8.308 | 1.00 | 37.23 | 6 | C |
| ATOM | 1104 | O | MET | B | 141 | 23.673 | 0.530 | 8.633 | 1.00 | 38.84 | 8 | O |
| ATOM | 1105 | CB | MET | B | 141 | 26.794 | 0.985 | 7.699 | 1.00 | 41.40 | 6 | C |
| ATOM | 1106 | CG | MET | B | 141 | 26.256 | 2.275 | 7.105 | 1.00 | 40.35 | 6 | C |
| ATOM | 1107 | SD | MET | B | 141 | 26.172 | 3.737 | 8.244 | 1.00 | 39.15 | 16 | S |
| ATOM | 1108 | CE | MET | B | 141 | 27.832 | 4.067 | 8.525 | 1.00 | 37.72 | 6 | C |
| ATOM | 1109 | N | ILE | B | 142 | 24.508 | −1.122 | 7.536 | 1.00 | 38.06 | 7 | N |
| ATOM | 1110 | CA | ILE | B | 142 | 23.200 | −1.595 | 7.040 | 1.00 | 37.74 | 6 | C |
| ATOM | 1111 | C | ILE | B | 142 | 22.199 | −1.917 | 8.162 | 1.00 | 36.93 | 6 | C |
| ATOM | 1112 | O | ILE | B | 142 | 21.016 | −1.641 | 8.045 | 1.00 | 35.06 | 8 | O |
| ATOM | 1113 | CB | ILE | B | 142 | 23.433 | −2.852 | 6.190 | 1.00 | 36.41 | 6 | C |
| ATOM | 1114 | CG1 | ILE | B | 142 | 24.232 | −2.333 | 4.997 | 1.00 | 36.64 | 6 | C |
| ATOM | 1115 | CG2 | ILE | B | 142 | 22.109 | −3.458 | 5.761 | 1.00 | 34.43 | 6 | C |
| ATOM | 1116 | CD1 | ILE | B | 142 | 24.719 | −3.358 | 4.027 | 1.00 | 35.31 | 6 | C |
| ATOM | 1117 | N | ALA | B | 143 | 22.690 | −2.447 | 9.259 | 1.00 | 36.65 | 7 | N |
| ATOM | 1118 | CA | ALA | B | 143 | 21.886 | −2.769 | 10.424 | 1.00 | 39.80 | 6 | C |
| ATOM | 1119 | C | ALA | B | 143 | 21.308 | −1.474 | 10.971 | 1.00 | 43.07 | 6 | C |
| ATOM | 1120 | O | ALA | B | 143 | 20.084 | −1.438 | 11.189 | 1.00 | 45.19 | 8 | O |
| ATOM | 1121 | CB | ALA | B | 143 | 22.663 | −3.504 | 11.517 | 1.00 | 36.00 | 6 | C |
| ATOM | 1122 | N | ILE | B | 144 | 22.161 | −0.456 | 11.096 | 1.00 | 44.32 | 7 | N |
| ATOM | 1123 | CA | ILE | B | 144 | 21.597 | 0.813 | 11.648 | 1.00 | 41.90 | 6 | C |
| ATOM | 1124 | C | ILE | B | 144 | 20.617 | 1.463 | 10.734 | 1.00 | 43.02 | 6 | C |
| ATOM | 1125 | O | ILE | B | 144 | 19.573 | 1.972 | 11.148 | 1.00 | 45.82 | 8 | O |
| ATOM | 1126 | CB | ILE | B | 144 | 22.739 | 1.787 | 11.926 | 1.00 | 40.70 | 6 | C |
| ATOM | 1127 | CG1 | ILE | B | 144 | 23.594 | 1.269 | 13.108 | 1.00 | 36.88 | 6 | C |
| ATOM | 1128 | CG2 | ILE | B | 144 | 22.095 | 3.120 | 12.214 | 1.00 | 40.48 | 6 | C |
| ATOM | 1129 | CD1 | ILE | B | 144 | 25.047 | 1.614 | 13.034 | 1.00 | 35.40 | 6 | C |
| ATOM | 1130 | N | LEU | B | 145 | 20.893 | 1.452 | 9.430 | 1.00 | 42.82 | 7 | N |
| ATOM | 1131 | CA | LEU | B | 145 | 19.932 | 2.090 | 8.509 | 1.00 | 43.51 | 6 | C |
| ATOM | 1132 | C | LEU | B | 145 | 18.599 | 1.365 | 8.614 | 1.00 | 47.49 | 6 | C |
| ATOM | 1133 | O | LEU | B | 145 | 17.539 | 1.980 | 8.466 | 1.00 | 47.82 | 8 | O |
| ATOM | 1134 | CB | LEU | B | 145 | 20.538 | 2.156 | 7.099 | 1.00 | 39.56 | 6 | C |
| ATOM | 1135 | CG | LEU | B | 145 | 21.733 | 3.104 | 6.962 | 1.00 | 40.75 | 6 | C |
| ATOM | 1136 | CD1 | LEU | B | 145 | 22.648 | 2.654 | 5.789 | 1.00 | 36.14 | 6 | C |
| ATOM | 1137 | CD2 | LEU | B | 145 | 21.226 | 4.557 | 6.726 | 1.00 | 39.09 | 6 | C |
| ATOM | 1138 | N | GLU | B | 146 | 18.658 | 0.032 | 8.830 | 1.00 | 50.74 | 7 | N |
| ATOM | 1139 | CA | GLU | B | 146 | 17.481 | −0.817 | 8.933 | 1.00 | 52.77 | 6 | C |
| ATOM | 1140 | C | GLU | B | 146 | 16.740 | −0.510 | 10.224 | 1.00 | 51.42 | 6 | C |
| ATOM | 1141 | O | GLU | B | 146 | 15.523 | −0.326 | 10.128 | 1.00 | 49.82 | 8 | O |
| ATOM | 1142 | CB | GLU | B | 146 | 17.824 | −2.291 | 8.862 | 1.00 | 56.62 | 6 | C |
| ATOM | 1143 | CG | GLU | B | 146 | 17.503 | −2.883 | 7.481 | 1.00 | 59.19 | 6 | C |
| ATOM | 1144 | CD | GLU | B | 146 | 18.348 | −4.087 | 7.089 | 1.00 | 61.12 | 6 | C |
| ATOM | 1145 | OE1 | GLU | B | 146 | 18.854 | −4.788 | 8.016 | 1.00 | 61.39 | 8 | O |
| ATOM | 1146 | OE2 | GLU | B | 146 | 18.522 | −4.355 | 5.874 | 1.00 | 60.98 | 8 | O |
| ATOM | 1147 | N | LYS | B | 147 | 17.495 | −0.352 | 11.316 | 1.00 | 50.01 | 7 | N |
| ATOM | 1148 | CA | LYS | B | 147 | 16.788 | 0.031 | 12.549 | 1.00 | 50.19 | 6 | C |
| ATOM | 1149 | C | LYS | B | 147 | 16.004 | 1.310 | 12.295 | 1.00 | 52.73 | 6 | C |
| ATOM | 1150 | O | LYS | B | 147 | 14.905 | 1.478 | 12.825 | 1.00 | 55.50 | 8 | O |
| ATOM | 1151 | CB | LYS | B | 147 | 17.752 | 0.223 | 13.687 | 1.00 | 48.73 | 6 | C |
| ATOM | 1152 | CG | LYS | B | 147 | 17.377 | 0.973 | 14.945 | 1.00 | 50.24 | 6 | C |
| ATOM | 1153 | CD | LYS | B | 147 | 18.689 | 1.254 | 15.705 | 1.00 | 50.76 | 6 | C |
| ATOM | 1154 | CE | LYS | B | 147 | 18.419 | 1.909 | 17.044 | 1.00 | 51.95 | 6 | C |
| ATOM | 1155 | NZ | LYS | B | 147 | 19.650 | 1.717 | 17.898 | 1.00 | 56.98 | 7 | N |
| ATOM | 1156 | N | HIS | B | 148 | 16.481 | 2.316 | 11.566 | 1.00 | 52.09 | 7 | N |
| ATOM | 1157 | CA | HIS | B | 148 | 15.725 | 3.560 | 11.364 | 1.00 | 49.25 | 6 | C |
| ATOM | 1158 | C | HIS | B | 148 | 14.849 | 3.550 | 10.121 | 1.00 | 48.50 | 6 | C |
| ATOM | 1159 | O | HIS | B | 148 | 14.448 | 4.608 | 9.636 | 1.00 | 47.26 | 8 | O |
| ATOM | 1160 | CB | HIS | B | 148 | 16.712 | 4.750 | 11.304 | 1.00 | 46.10 | 6 | C |
| ATOM | 1161 | CG | HIS | B | 148 | 17.424 | 4.879 | 12.623 | 1.00 | 41.85 | 6 | C |
| ATOM | 1162 | ND1 | HIS | B | 148 | 16.847 | 5.506 | 13.690 | 1.00 | 42.90 | 7 | N |
| ATOM | 1163 | CD2 | HIS | B | 148 | 18.613 | 4.482 | 13.049 | 1.00 | 41.74 | 6 | C |
| ATOM | 1164 | CE1 | HIS | B | 148 | 17.678 | 5.486 | 14.745 | 1.00 | 43.19 | 6 | C |
| ATOM | 1165 | NE2 | HIS | B | 148 | 18.774 | 4.855 | 14.353 | 1.00 | 43.09 | 7 | N |
| ATOM | 1166 | N | GLY | B | 149 | 14.583 | 2.348 | 9.559 | 1.00 | 45.98 | 7 | N |
| ATOM | 1167 | CA | GLY | B | 149 | 13.724 | 2.336 | 8.392 | 1.00 | 46.52 | 6 | C |
| ATOM | 1168 | C | GLY | B | 149 | 14.280 | 2.707 | 7.052 | 1.00 | 47.20 | 6 | C |
| ATOM | 1169 | O | GLY | B | 149 | 13.435 | 2.950 | 6.183 | 1.00 | 48.67 | 8 | O |
| ATOM | 1170 | N | LEU | B | 150 | 15.572 | 2.794 | 6.876 | 1.00 | 46.03 | 7 | N |
| ATOM | 1171 | CA | LEU | B | 150 | 16.104 | 3.168 | 5.568 | 1.00 | 46.32 | 6 | C |
| ATOM | 1172 | C | LEU | B | 150 | 16.706 | 1.882 | 4.992 | 1.00 | 47.26 | 6 | C |
| ATOM | 1173 | O | LEU | B | 150 | 16.355 | 0.880 | 5.620 | 1.00 | 48.53 | 8 | O |
| ATOM | 1174 | CB | LEU | B | 150 | 17.148 | 4.279 | 5.762 | 1.00 | 44.83 | 6 | C |
| ATOM | 1175 | CG | LEU | B | 150 | 16.748 | 5.576 | 6.428 | 1.00 | 44.34 | 6 | C |
| ATOM | 1176 | CD1 | LEU | B | 150 | 17.903 | 6.303 | 7.072 | 1.00 | 45.87 | 6 | C |
| ATOM | 1177 | CD2 | LEU | B | 150 | 15.983 | 6.490 | 5.527 | 1.00 | 43.42 | 6 | C |
| ATOM | 1178 | N | SER | B | 151 | 17.500 | 1.814 | 3.941 | 1.00 | 46.48 | 7 | N |
| ATOM | 1179 | CA | SER | B | 151 | 18.093 | 0.551 | 3.484 | 1.00 | 47.24 | 6 | C |
| ATOM | 1180 | C | SER | B | 151 | 19.518 | 0.736 | 2.950 | 1.00 | 47.78 | 6 | C |
| ATOM | 1181 | O | SER | B | 151 | 19.852 | 1.843 | 2.539 | 1.00 | 51.22 | 8 | O |
| ATOM | 1182 | CB | SER | B | 151 | 17.232 | −0.203 | 2.520 | 1.00 | 44.47 | 6 | C |
| ATOM | 1183 | CG | SER | B | 151 | 16.836 | 0.577 | 1.408 | 1.00 | 44.70 | 8 | O |
| ATOM | 1184 | N | GLY | B | 152 | 20.402 | −0.214 | 2.970 | 1.00 | 43.86 | 7 | N |
| ATOM | 1185 | CA | GLY | B | 152 | 21.765 | −0.145 | 2.465 | 1.00 | 46.38 | 6 | C |
| ATOM | 1186 | C | GLY | B | 152 | 22.101 | −1.397 | 1.633 | 1.00 | 48.12 | 6 | C |
| ATOM | 1187 | O | GLY | B | 152 | 21.290 | −2.315 | 1.627 | 1.00 | 47.50 | 8 | O |
| ATOM | 1188 | N | SER | B | 153 | 23.194 | −1.440 | 0.890 | 1.00 | 49.94 | 7 | N |
| ATOM | 1189 | CA | SER | B | 153 | 23.667 | −2.546 | 0.067 | 1.00 | 50.27 | 6 | C |
| ATOM | 1190 | C | SER | B | 153 | 25.115 | −2.318 | −0.290 | 1.00 | 52.71 | 6 | C |
| ATOM | 1191 | O | SER | B | 153 | 25.577 | −1.162 | −0.234 | 1.00 | 57.22 | 8 | O |
| ATOM | 1192 | CB | SER | B | 153 | 22.824 | −2.849 | −1.153 | 1.00 | 48.63 | 6 | C |
| ATOM | 1193 | OG | SER | B | 153 | 22.566 | −1.664 | −1.883 | 1.00 | 47.38 | 8 | O |
| ATOM | 1194 | N | VAL | B | 154 | 25.901 | −3.322 | −0.596 | 1.00 | 54.95 | 7 | N |
| ATOM | 1195 | CA | VAL | B | 154 | 27.336 | −3.116 | −0.887 | 1.00 | 54.88 | 6 | C |
| ATOM | 1196 | C | VAL | B | 154 | 27.617 | −3.139 | −2.367 | 1.00 | 55.13 | 6 | C |
| ATOM | 1197 | O | VAL | B | 154 | 26.864 | −3.703 | −3.163 | 1.00 | 53.23 | 8 | O |
| ATOM | 1198 | CB | VAL | B | 154 | 28.212 | −4.159 | −0.190 | 1.00 | 55.62 | 6 | C |
| ATOM | 1199 | CG1 | VAL | B | 154 | 29.612 | −3.586 | 0.003 | 1.00 | 56.32 | 6 | C |
| ATOM | 1200 | CG2 | VAL | B | 154 | 27.692 | −4.574 | 1.188 | 1.00 | 56.49 | 6 | C |
| ATOM | 1201 | N | VAL | B | 155 | 28.670 | −2.472 | −2.846 | 1.00 | 55.15 | 7 | N |
| ATOM | 1202 | CA | VAL | B | 155 | 28.984 | −2.478 | −4.288 | 1.00 | 52.00 | 6 | C |
| ATOM | 1203 | C | VAL | B | 155 | 30.449 | −2.854 | −4.407 | 1.00 | 55.08 | 6 | C |
| ATOM | 1204 | O | VAL | B | 155 | 30.998 | −2.354 | −3.398 | 1.00 | 54.21 | 8 | O |
| ATOM | 1205 | CB | VAL | B | 155 | 28.940 | −1.031 | −4.768 | 1.00 | 50.77 | 6 | C |
| ATOM | 1206 | CG1 | VAL | B | 155 | 29.469 | −0.843 | −6.184 | 1.00 | 47.68 | 6 | C |
| ATOM | 1207 | CG2 | VAL | B | 155 | 27.525 | −0.520 | −4.579 | 1.00 | 48.00 | 6 | C |
| ATOM | 1208 | N | ARG | B | 156 | 31.082 | −3.547 | −5.434 | 1.00 | 57.38 | 7 | N |
| ATOM | 1209 | CA | ARG | B | 156 | 32.555 | −3.565 | −5.049 | 1.00 | 55.68 | 6 | C |
| ATOM | 1210 | C | ARG | B | 156 | 33.447 | −2.756 | −5.919 | 1.00 | 53.31 | 6 | C |
| ATOM | 1211 | O | ARG | B | 156 | 33.120 | −2.361 | −7.051 | 1.00 | 56.50 | 8 | O |
| ATOM | 1212 | CB | ARG | B | 156 | 33.150 | −4.854 | −4.569 | 1.00 | 56.87 | 6 | C |
| ATOM | 1213 | CG | ARG | B | 156 | 32.504 | −5.541 | −3.382 | 1.00 | 52.67 | 6 | C |
| ATOM | 1214 | CD | ARG | B | 156 | 33.295 | −6.889 | −3.023 | 0.00 | 25.00 | 6 | C |
| ATOM | 1215 | NE | ARG | B | 156 | 34.550 | −6.503 | −2.386 | 0.00 | 25.00 | 7 | N |
| ATOM | 1216 | CZ | ARG | B | 156 | 35.487 | −7.364 | −2.001 | 0.00 | 25.00 | 6 | C |
| ATOM | 1217 | NH1 | ARG | B | 156 | 35.312 | −8.663 | −2.191 | 0.00 | 25.00 | 7 | N |
| ATOM | 1218 | NH2 | ARG | B | 156 | 36.599 | −6.920 | −1.430 | 0.00 | 25.00 | 7 | N |
| ATOM | 1219 | N | LEU | B | 157 | 34.486 | −2.736 | −4.703 | 0.00 | 25.00 | 7 | N |
| ATOM | 1220 | CA | LEU | B | 157 | 35.407 | −1.837 | −5.405 | 0.00 | 25.00 | 6 | C |
| ATOM | 1221 | C | LEU | B | 157 | 36.850 | −2.394 | −5.449 | 0.00 | 25.00 | 6 | C |
| ATOM | 1222 | O | LEU | B | 157 | 37.217 | −3.171 | −4.565 | 0.00 | 25.00 | 8 | O |
| ATOM | 1223 | CB | LEU | B | 157 | 35.591 | −0.511 | −4.699 | 0.00 | 25.00 | 6 | C |
| ATOM | 1224 | CG | LEU | B | 157 | 34.618 | 0.306 | −3.909 | 0.00 | 25.00 | 6 | C |
| ATOM | 1225 | CD1 | LEU | B | 157 | 35.436 | 1.147 | −2.911 | 0.00 | 25.00 | 6 | C |
| ATOM | 1226 | CD2 | LEU | B | 157 | 33.802 | 1.251 | −4.802 | 0.00 | 25.00 | 6 | C |
| ATOM | 1227 | N | GLU | B | 158 | 37.627 | −1.922 | −6.442 | 0.00 | 25.00 | 7 | N |
| ATOM | 1228 | CA | GLU | B | 158 | 39.029 | −2.353 | −6.518 | 0.00 | 25.00 | 6 | C |
| ATOM | 1229 | C | GLU | B | 158 | 40.049 | −1.289 | −6.954 | 0.00 | 25.00 | 6 | C |
| ATOM | 1230 | O | GLU | B | 158 | 41.197 | −1.281 | −6.481 | 0.00 | 25.00 | 8 | O |
| ATOM | 1231 | CB | GLU | B | 158 | 39.250 | −3.521 | −7.519 | 0.00 | 25.00 | 6 | C |
| ATOM | 1232 | CG | GLU | B | 158 | 38.759 | −4.665 | −7.077 | 0.00 | 25.00 | 6 | C |
| ATOM | 1233 | CD | GLU | B | 158 | 39.008 | −5.794 | −8.067 | 0.00 | 25.00 | 6 | C |
| ATOM | 1234 | OE1 | GLU | B | 158 | 39.498 | −5.518 | −9.182 | 0.00 | 25.00 | 8 | O |
| ATOM | 1235 | OE2 | GLU | B | 158 | 38.709 | −6.962 | −7.725 | 0.00 | 25.00 | 8 | O |
| ATOM | 1236 | N | LYS | B | 159 | 39.673 | −0.415 | −7.891 | 0.00 | 25.00 | 7 | N |
| ATOM | 1237 | CA | LYS | B | 159 | 40.600 | 0.575 | −8.425 | 0.00 | 25.00 | 6 | C |
| ATOM | 1238 | C | LYS | B | 159 | 40.434 | 1.919 | −7.724 | 0.00 | 25.00 | 6 | C |
| ATOM | 1239 | O | LYS | B | 159 | 41.274 | 2.808 | −8.005 | 0.00 | 25.00 | 8 | O |
| ATOM | 1240 | CB | LYS | B | 159 | 40.399 | 0.740 | −9.933 | 0.00 | 25.00 | 6 | C |
| ATOM | 1241 | CG | LYS | B | 159 | 40.782 | −0.485 | −10.747 | 0.00 | 25.00 | 6 | C |
| ATOM | 1242 | CD | LYS | B | 159 | 40.558 | −0.254 | −12.232 | 0.00 | 25.00 | 6 | C |
| ATOM | 1243 | CE | LYS | B | 159 | 40.940 | −1.479 | −13.046 | 0.00 | 25.00 | 6 | C |
| ATOM | 1244 | NZ | LYS | B | 159 | 40.726 | −1.266 | −14.505 | 0.00 | 25.00 | 7 | N |
| ATOM | 1245 | N | VAL | B | 160 | 39.386 | 2.069 | −6.890 | 0.00 | 25.00 | 7 | N |
| ATOM | 1246 | CA | VAL | B | 160 | 39.288 | 3.395 | −6.269 | 0.00 | 25.00 | 6 | C |
| ATOM | 1247 | C | VAL | B | 160 | 39.276 | 3.316 | −4.735 | 0.00 | 25.00 | 6 | C |
| ATOM | 1248 | O | VAL | B | 160 | 38.902 | 2.289 | −4.154 | 0.00 | 25.00 | 8 | O |
| ATOM | 1249 | CB | VAL | B | 160 | 38.134 | 4.261 | −6.791 | 0.00 | 25.00 | 6 | C |
| ATOM | 1250 | CG1 | VAL | B | 160 | 38.642 | 5.113 | −7.964 | 0.00 | 25.00 | 6 | C |
| ATOM | 1251 | CG2 | VAL | B | 160 | 36.906 | 3.427 | −7.161 | 0.00 | 25.00 | 6 | C |
| ATOM | 1252 | N | LEU | B | 161 | 39.695 | 4.458 | −4.153 | 0.00 | 25.00 | 7 | N |
| ATOM | 1253 | CA | LEU | B | 161 | 39.745 | 4.444 | −2.696 | 0.00 | 25.00 | 6 | C |
| ATOM | 1254 | C | LEU | B | 161 | 38.343 | 4.411 | −2.098 | 0.00 | 25.00 | 6 | C |
| ATOM | 1255 | O | LEU | B | 161 | 38.074 | 3.737 | −1.097 | 0.00 | 25.00 | 8 | O |
| ATOM | 1256 | CB | LEU | B | 161 | 40.511 | 5.660 | −2.173 | 0.00 | 25.00 | 6 | C |
| ATOM | 1257 | CG | LEU | B | 161 | 41.998 | 5.729 | −2.529 | 0.00 | 25.00 | 6 | C |
| ATOM | 1258 | CD1 | LEU | B | 161 | 42.600 | 7.050 | −2.076 | 0.00 | 25.00 | 6 | C |
| ATOM | 1259 | CD2 | LEU | B | 161 | 42.764 | 4.602 | −1.853 | 0.00 | 25.00 | 6 | C |
| ATOM | 1260 | N | HIS | B | 162 | 37.559 | 4.933 | −2.633 | 1.00 | 52.56 | 7 | N |
| ATOM | 1261 | CA | HIS | B | 162 | 36.220 | 5.320 | −2.212 | 1.00 | 54.49 | 6 | C |
| ATOM | 1262 | C | HIS | B | 162 | 35.194 | 5.133 | −3.348 | 1.00 | 52.36 | 6 | C |
| ATOM | 1263 | O | HIS | B | 162 | 35.531 | 5.227 | −4.513 | 1.00 | 50.07 | 8 | O |
| ATOM | 1264 | CB | HIS | B | 162 | 36.041 | 6.746 | −1.701 | 1.00 | 58.47 | 6 | C |
| ATOM | 1265 | CG | HIS | B | 162 | 36.592 | 7.109 | −0.361 | 1.00 | 62.11 | 6 | C |
| ATOM | 1266 | ND1 | HIS | B | 162 | 36.835 | 8.400 | 0.060 | 1.00 | 62.70 | 7 | N |
| ATOM | 1267 | CD2 | HIS | B | 162 | 36.954 | 6.317 | 0.673 | 1.00 | 63.47 | 6 | C |
| ATOM | 1268 | CE1 | HIS | B | 162 | 37.323 | 8.375 | 1.294 | 1.00 | 63.10 | 6 | C |
| ATOM | 1269 | NE2 | HIS | B | 162 | 37.403 | 7.136 | 1.686 | 1.00 | 62.82 | 7 | N |
| ATOM | 1270 | N | LEU | B | 163 | 33.940 | 4.880 | −3.005 | 1.00 | 50.09 | 7 | N |
| ATOM | 1271 | CA | LEU | B | 163 | 32.947 | 4.713 | −4.072 | 1.00 | 48.67 | 6 | C |
| ATOM | 1272 | C | LEU | B | 163 | 32.824 | 6.011 | −4.819 | 1.00 | 48.94 | 6 | C |
| ATOM | 1273 | O | LEU | B | 163 | 32.860 | 6.171 | −6.052 | 1.00 | 51.81 | 8 | O |
| ATOM | 1274 | CB | LEU | B | 163 | 31.638 | 4.314 | −3.398 | 1.00 | 48.84 | 6 | C |
| ATOM | 1275 | CG | LEU | B | 163 | 30.359 | 4.101 | −4.229 | 1.00 | 48.55 | 6 | C |
| ATOM | 1276 | CD1 | LEU | B | 163 | 30.364 | 2.786 | −4.972 | 1.00 | 43.41 | 6 | C |
| ATOM | 1277 | CD2 | LEU | B | 163 | 29.161 | 4.172 | −3.284 | 1.00 | 45.08 | 6 | C |
| ATOM | 1278 | N | LYS | B | 164 | 32.686 | 7.109 | −4.071 | 1.00 | 47.99 | 7 | N |
| ATOM | 1279 | CA | LYS | B | 164 | 32.450 | 8.452 | −4.626 | 1.00 | 46.06 | 6 | C |
| ATOM | 1280 | C | LYS | B | 164 | 33.584 | 9.062 | −5.380 | 1.00 | 46.34 | 6 | C |
| ATOM | 1281 | O | LYS | B | 164 | 33.432 | 10.123 | −6.011 | 1.00 | 47.44 | 8 | O |
| ATOM | 1282 | CB | LYS | B | 164 | 31.754 | 9.297 | −3.552 | 1.00 | 45.20 | 6 | C |
| ATOM | 1283 | CG | LYS | B | 164 | 30.355 | 8.675 | −3.339 | 1.00 | 46.17 | 6 | C |
| ATOM | 1284 | CD | LYS | B | 164 | 29.218 | 9.472 | −2.730 | 1.00 | 42.78 | 6 | C |
| ATOM | 1285 | CE | LYS | B | 164 | 29.771 | 10.218 | −1.514 | 1.00 | 42.61 | 6 | C |
| ATOM | 1286 | NZ | LYS | B | 164 | 28.804 | 10.670 | −0.482 | 1.00 | 38.08 | 7 | N |
| ATOM | 1287 | N | THR | B | 165 | 34.725 | 8.395 | −5.411 | 1.00 | 44.07 | 7 | N |
| ATOM | 1288 | CA | THR | B | 165 | 35.785 | 8.905 | −6.255 | 1.00 | 43.47 | 6 | C |
| ATOM | 1289 | C | THR | B | 165 | 35.181 | 8.737 | −7.637 | 1.00 | 44.45 | 6 | C |
| ATOM | 1290 | O | THR | B | 165 | 35.205 | 9.677 | −8.450 | 1.00 | 48.41 | 8 | O |
| ATOM | 1291 | CB | THR | B | 165 | 37.024 | 8.067 | −5.978 | 1.00 | 44.53 | 6 | C |
| ATOM | 1292 | OG1 | THR | B | 165 | 37.305 | 8.284 | −4.588 | 1.00 | 46.53 | 8 | O |
| ATOM | 1293 | CG2 | THR | B | 165 | 38.196 | 8.433 | −6.848 | 1.00 | 45.02 | 6 | C |
| ATOM | 1294 | N | GLY | B | 166 | 34.598 | 7.611 | −8.015 | 1.00 | 44.45 | 7 | N |
| ATOM | 1295 | CA | GLY | B | 166 | 34.013 | 7.458 | −9.344 | 1.00 | 45.59 | 6 | C |
| ATOM | 1296 | C | GLY | B | 166 | 32.556 | 7.610 | −9.596 | 1.00 | 47.19 | 6 | C |
| ATOM | 1297 | O | GLY | B | 166 | 32.087 | 7.413 | −10.749 | 1.00 | 51.07 | 8 | O |
| ATOM | 1298 | N | LEU | B | 167 | 31.692 | 8.018 | −8.633 | 1.00 | 46.82 | 7 | N |
| ATOM | 1299 | CA | LEU | B | 167 | 30.264 | 8.179 | −8.922 | 1.00 | 41.85 | 6 | C |
| ATOM | 1300 | C | LEU | B | 167 | 29.683 | 9.385 | −8.211 | 1.00 | 41.55 | 6 | C |
| ATOM | 1301 | O | LEU | B | 167 | 30.299 | 9.806 | −7.237 | 1.00 | 45.19 | 8 | O |
| ATOM | 1302 | CB | LEU | B | 167 | 29.401 | 7.074 | −8.253 | 1.00 | 41.52 | 6 | C |
| ATOM | 1303 | CG | LEU | B | 167 | 29.463 | 5.666 | −8.785 | 1.00 | 39.71 | 6 | C |
| ATOM | 1304 | CD1 | LEU | B | 167 | 29.165 | 4.592 | −7.794 | 1.00 | 37.38 | 6 | C |
| ATOM | 1305 | CD2 | LEU | B | 167 | 28.434 | 5.548 | −9.911 | 1.00 | 43.45 | 6 | C |
| ATOM | 1306 | N | ALA | B | 168 | 28.487 | 9.801 | −8.618 | 1.00 | 37.23 | 7 | N |
| ATOM | 1307 | CA | ALA | B | 168 | 27.843 | 10.915 | −7.992 | 1.00 | 32.18 | 6 | C |
| ATOM | 1308 | C | ALA | B | 168 | 26.359 | 10.897 | −8.395 | 1.00 | 34.79 | 6 | C |
| ATOM | 1309 | O | ALA | B | 168 | 26.038 | 10.939 | −9.603 | 1.00 | 34.61 | 8 | O |
| ATOM | 1310 | CB | ALA | B | 168 | 28.421 | 12.258 | −8.383 | 1.00 | 32.00 | 6 | C |
| ATOM | 1311 | N | TYR | B | 169 | 25.496 | 10.858 | −7.370 | 1.00 | 31.50 | 7 | N |
| ATOM | 1312 | CA | TYR | B | 169 | 24.078 | 10.830 | −7.643 | 1.00 | 32.04 | 6 | C |
| ATOM | 1313 | C | TYR | B | 169 | 23.625 | 12.249 | −7.654 | 1.00 | 34.26 | 6 | C |
| ATOM | 1314 | O | TYR | B | 169 | 23.795 | 12.948 | −6.626 | 1.00 | 36.38 | 8 | O |
| ATOM | 1315 | CB | TYR | B | 169 | 23.268 | 10.180 | −6.490 | 1.00 | 35.64 | 6 | C |
| ATOM | 1316 | CG | TYR | B | 169 | 21.803 | 10.136 | −6.824 | 1.00 | 34.75 | 6 | C |
| ATOM | 1317 | CD1 | TYR | B | 169 | 21.447 | 9.658 | −8.112 | 1.00 | 36.15 | 6 | C |
| ATOM | 1318 | CD2 | TYR | B | 169 | 20.806 | 10.517 | −5.986 | 1.00 | 33.59 | 6 | C |
| ATOM | 1319 | CE1 | TYR | B | 169 | 20.142 | 9.577 | −8.521 | 1.00 | 32.27 | 6 | C |
| ATOM | 1320 | CE2 | TYR | B | 169 | 19.491 | 10.448 | −6.373 | 1.00 | 33.06 | 6 | C |
| ATOM | 1321 | CZ | TYR | B | 169 | 19.174 | 9.981 | −7.624 | 1.00 | 31.84 | 6 | C |
| ATOM | 1322 | OH | TYR | B | 169 | 17.850 | 9.924 | −7.974 | 1.00 | 33.37 | 8 | O |
| ATOM | 1323 | N | LEU | B | 170 | 23.029 | 12.715 | −8.747 | 1.00 | 34.58 | 7 | N |
| ATOM | 1324 | CA | LEU | B | 170 | 22.617 | 14.100 | −8.800 | 1.00 | 32.70 | 6 | C |
| ATOM | 1325 | C | LEU | B | 170 | 21.161 | 14.323 | −8.677 | 1.00 | 33.53 | 6 | C |
| ATOM | 1326 | O | LEU | B | 170 | 20.636 | 15.383 | −9.119 | 1.00 | 35.20 | 8 | O |
| ATOM | 1327 | CB | LEU | B | 170 | 23.158 | 14.816 | −10.045 | 1.00 | 32.24 | 6 | C |
| ATOM | 1328 | CG | LEU | B | 170 | 24.695 | 14.707 | −10.225 | 1.00 | 33.05 | 6 | C |
| ATOM | 1329 | CD1 | LEU | B | 170 | 24.981 | 15.327 | −11.589 | 1.00 | 31.64 | 6 | C |
| ATOM | 1330 | CD2 | LEU | B | 170 | 25.522 | 15.275 | −9.107 | 1.00 | 24.18 | 6 | C |
| ATOM | 1331 | N | GLU | B | 171 | 20.541 | 13.354 | −8.011 | 1.00 | 36.79 | 7 | N |
| ATOM | 1332 | CA | GLU | B | 171 | 19.084 | 13.393 | −7.856 | 1.00 | 38.36 | 6 | C |
| ATOM | 1333 | C | GLU | B | 171 | 18.466 | 13.189 | −9.252 | 1.00 | 41.79 | 6 | C |
| ATOM | 1334 | O | GLU | B | 171 | 18.947 | 13.238 | −10.375 | 1.00 | 39.49 | 8 | O |
| ATOM | 1335 | CB | GLU | B | 171 | 18.562 | 14.650 | −7.200 | 1.00 | 35.96 | 6 | C |
| ATOM | 1336 | CG | GLU | B | 171 | 18.971 | 14.890 | −5.768 | 1.00 | 37.63 | 6 | C |
| ATOM | 1337 | CD | GLU | B | 171 | 18.696 | 13.699 | −4.871 | 1.00 | 39.41 | 6 | C |
| ATOM | 1338 | OE1 | GLU | B | 171 | 17.513 | 13.227 | −4.838 | 1.00 | 36.67 | 8 | O |
| ATOM | 1339 | OE2 | GLU | B | 171 | 19.652 | 13.251 | −4.214 | 1.00 | 42.60 | 8 | O |
| ATOM | 1340 | N | HIS | B | 172 | 17.157 | 12.898 | −9.334 | 1.00 | 47.10 | 7 | N |
| ATOM | 1341 | CA | HIS | B | 172 | 16.367 | 12.707 | −10.541 | 1.00 | 49.24 | 6 | C |
| ATOM | 1342 | C | HIS | B | 172 | 16.812 | 11.508 | −11.364 | 1.00 | 51.04 | 6 | C |
| ATOM | 1343 | O | HIS | B | 172 | 16.782 | 11.369 | −12.604 | 1.00 | 53.68 | 8 | O |
| ATOM | 1344 | CB | HIS | B | 172 | 16.479 | 14.022 | −11.365 | 1.00 | 49.14 | 6 | C |
| ATOM | 1345 | CG | HIS | B | 172 | 16.044 | 15.236 | −10.584 | 1.00 | 47.23 | 6 | C |
| ATOM | 1346 | ND1 | HIS | B | 172 | 14.832 | 15.400 | −10.038 | 1.00 | 46.91 | 7 | N |
| ATOM | 1347 | CD2 | HIS | B | 172 | 16.719 | 16.369 | −10.265 | 1.00 | 45.53 | 6 | C |
| ATOM | 1348 | CE1 | HIS | B | 172 | 14.749 | 16.573 | −9.407 | 1.00 | 46.65 | 6 | C |
| ATOM | 1349 | NE2 | HIS | B | 172 | 15.921 | 17.190 | −9.543 | 1.00 | 44.49 | 7 | N |
| ATOM | 1350 | N | ASN | B | 173 | 17.394 | 10.524 | −10.697 | 1.00 | 50.07 | 7 | N |
| ATOM | 1351 | CA | ASN | B | 173 | 17.913 | 9.329 | −11.339 | 1.00 | 51.48 | 6 | C |
| ATOM | 1352 | C | ASN | B | 173 | 19.106 | 9.640 | −12.202 | 1.00 | 47.82 | 6 | C |
| ATOM | 1353 | O | ASN | B | 173 | 19.653 | 8.758 | −12.843 | 1.00 | 50.44 | 8 | O |
| ATOM | 1354 | CB | ASN | B | 173 | 16.813 | 8.582 | −12.104 | 1.00 | 55.02 | 6 | C |
| ATOM | 1355 | CG | ASN | B | 173 | 17.007 | 7.081 | −11.926 | 1.00 | 59.02 | 6 | C |
| ATOM | 1356 | OD1 | ASN | B | 173 | 18.019 | 6.462 | −12.212 | 1.00 | 59.24 | 8 | O |
| ATOM | 1357 | ND2 | ASN | B | 173 | 15.957 | 6.455 | −11.409 | 1.00 | 63.60 | 7 | N |
| ATOM | 1358 | N | ASN | B | 174 | 19.636 | 10.826 | −12.167 | 1.00 | 46.41 | 7 | N |
| ATOM | 1359 | CA | ASN | B | 174 | 20.826 | 11.232 | −12.876 | 1.00 | 47.11 | 6 | C |
| ATOM | 1360 | C | ASN | B | 174 | 22.038 | 10.719 | −12.095 | 1.00 | 44.22 | 6 | C |
| ATOM | 1361 | O | ASN | B | 174 | 22.076 | 10.892 | −10.902 | 1.00 | 45.66 | 8 | O |
| ATOM | 1362 | CB | ASN | B | 174 | 20.846 | 12.755 | −13.090 | 1.00 | 47.08 | 6 | C |
| ATOM | 1363 | CG | ASN | B | 174 | 19.692 | 13.186 | −13.983 | 1.00 | 49.01 | 6 | C |
| ATOM | 1364 | OD1 | ASN | B | 174 | 19.582 | 12.477 | −15.004 | 1.00 | 51.12 | 8 | O |
| ATOM | 1365 | ND2 | ASN | B | 174 | 18.922 | 14.209 | −13.657 | 1.00 | 46.23 | 7 | N |
| ATOM | 1366 | N | LEU | B | 175 | 22.989 | 10.074 | −12.735 | 1.00 | 42.95 | 7 | N |
| ATOM | 1367 | CA | LEU | B | 175 | 24.152 | 9.623 | −11.997 | 1.00 | 41.94 | 6 | C |
| ATOM | 1368 | C | LEU | B | 175 | 25.403 | 9.700 | −12.831 | 1.00 | 43.63 | 6 | C |
| ATOM | 1369 | O | LEU | B | 175 | 25.401 | 9.071 | −13.896 | 1.00 | 50.53 | 8 | O |
| ATOM | 1370 | CB | LEU | B | 175 | 23.908 | 8.236 | −11.460 | 1.00 | 40.84 | 6 | C |
| ATOM | 1371 | CG | LEU | B | 175 | 25.137 | 7.535 | −10.971 | 1.00 | 42.62 | 6 | C |
| ATOM | 1372 | CD1 | LEU | B | 175 | 24.976 | 6.760 | −9.709 | 1.00 | 42.82 | 6 | C |
| ATOM | 1373 | CD2 | LEU | B | 175 | 25.447 | 6.523 | −12.108 | 1.00 | 47.08 | 6 | C |
| ATOM | 1374 | N | LEU | B | 176 | 26.424 | 10.393 | −12.369 | 1.00 | 40.42 | 7 | N |
| ATOM | 1375 | CA | LEU | B | 176 | 27.712 | 10.476 | −12.989 | 1.00 | 39.12 | 6 | C |
| ATOM | 1376 | C | LEU | B | 176 | 28.497 | 9.196 | −12.713 | 1.00 | 40.42 | 6 | C |
| ATOM | 1377 | O | LEU | B | 176 | 28.381 | 8.701 | −11.569 | 1.00 | 41.65 | 8 | O |
| ATOM | 1378 | CB | LEU | B | 176 | 28.507 | 11.637 | −12.394 | 1.00 | 37.03 | 6 | C |
| ATOM | 1379 | CG | LEU | B | 176 | 27.933 | 13.041 | −12.528 | 1.00 | 35.10 | 6 | C |
| ATOM | 1380 | CD1 | LEU | B | 176 | 29.054 | 14.032 | −12.302 | 1.00 | 35.04 | 6 | C |
| ATOM | 1381 | CD2 | LEU | B | 176 | 27.208 | 13.356 | −13.814 | 1.00 | 33.65 | 6 | C |
| ATOM | 1382 | N | ALA | B | 177 | 29.269 | 8.699 | −13.693 | 1.00 | 43.13 | 7 | N |
| ATOM | 1383 | CA | ALA | B | 177 | 30.026 | 7.455 | −13.443 | 1.00 | 45.69 | 6 | C |
| ATOM | 1384 | C | ALA | B | 177 | 31.299 | 7.396 | −14.270 | 1.00 | 48.44 | 6 | C |
| ATOM | 1385 | O | ALA | B | 177 | 31.232 | 7.820 | −15.430 | 1.00 | 53.06 | 8 | O |
| ATOM | 1386 | CB | ALA | B | 177 | 29.267 | 6.165 | −13.754 | 1.00 | 39.09 | 6 | C |
| ATOM | 1387 | N | ALA | B | 178 | 32.328 | 6.845 | −13.678 | 1.00 | 49.97 | 7 | N |
| ATOM | 1388 | CA | ALA | B | 178 | 33.614 | 6.714 | −14.328 | 1.00 | 53.79 | 6 | C |
| ATOM | 1389 | C | ALA | B | 178 | 34.432 | 5.484 | −13.949 | 1.00 | 57.76 | 6 | C |
| ATOM | 1390 | O | ALA | B | 178 | 34.658 | 5.243 | −12.729 | 1.00 | 61.29 | 8 | O |
| ATOM | 1391 | CB | ALA | B | 178 | 34.422 | 7.922 | −13.784 | 1.00 | 48.20 | 6 | C |
| ATOM | 1392 | N | GLY | B | 179 | 34.997 | 4.767 | −14.919 | 1.00 | 59.84 | 7 | N |
| ATOM | 1393 | CA | GLY | B | 179 | 35.857 | 3.601 | −14.619 | 1.00 | 59.80 | 6 | C |
| ATOM | 1394 | C | GLY | B | 179 | 34.977 | 2.381 | −14.325 | 1.00 | 60.32 | 6 | C |
| ATOM | 1395 | O | GLY | B | 179 | 33.942 | 2.252 | −14.995 | 1.00 | 60.67 | 8 | O |
| ATOM | 1396 | N | GLU | B | 180 | 35.370 | 1.556 | −13.345 | 1.00 | 59.69 | 7 | N |
| ATOM | 1397 | CA | GLU | B | 180 | 34.513 | 0.409 | −13.032 | 1.00 | 61.12 | 6 | C |
| ATOM | 1398 | C | GLU | B | 180 | 33.057 | 0.792 | −13.020 | 1.00 | 59.99 | 6 | C |
| ATOM | 1399 | O | GLU | B | 180 | 32.323 | 0.462 | −13.939 | 1.00 | 65.83 | 8 | O |
| ATOM | 1400 | CB | GLU | B | 180 | 34.746 | −0.294 | −11.693 | 1.00 | 63.18 | 6 | C |
| ATOM | 1401 | CG | GLU | B | 180 | 35.928 | 0.165 | −10.933 | 1.00 | 68.29 | 6 | C |
| ATOM | 1402 | CD | GLU | B | 180 | 36.335 | −0.341 | −9.582 | 1.00 | 71.72 | 6 | C |
| ATOM | 1403 | OE1 | GLU | B | 180 | 35.618 | −1.117 | −8.889 | 1.00 | 73.73 | 8 | O |
| ATOM | 1404 | OE2 | GLU | B | 180 | 37.477 | 0.102 | −9.224 | 1.00 | 72.41 | 8 | O |
| ATOM | 1405 | N | PHE | B | 181 | 32.451 | 1.538 | −12.136 | 1.00 | 57.73 | 7 | N |
| ATOM | 1406 | CA | PHE | B | 181 | 31.030 | 1.887 | −12.148 | 1.00 | 53.86 | 6 | C |
| ATOM | 1407 | C | PHE | B | 181 | 30.346 | 2.204 | −13.437 | 1.00 | 52.34 | 6 | C |
| ATOM | 1408 | O | PHE | B | 181 | 29.092 | 2.289 | −13.351 | 1.00 | 55.27 | 8 | O |
| ATOM | 1409 | CB | PHE | B | 181 | 30.723 | 3.063 | −11.176 | 1.00 | 48.61 | 6 | C |
| ATOM | 1410 | CG | PHE | B | 181 | 31.653 | 2.968 | −10.006 | 1.00 | 44.43 | 6 | C |
| ATOM | 1411 | CD1 | PHE | B | 181 | 32.711 | 3.801 | −9.846 | 1.00 | 43.88 | 6 | C |
| ATOM | 1412 | CD2 | PHE | B | 181 | 31.408 | 1.987 | −9.069 | 1.00 | 44.07 | 6 | C |
| ATOM | 1413 | CE1 | PHE | B | 181 | 33.539 | 3.686 | −8.759 | 1.00 | 44.19 | 6 | C |
| ATOM | 1414 | CE2 | PHE | B | 181 | 32.216 | 1.833 | −7.968 | 1.00 | 41.53 | 6 | C |
| ATOM | 1415 | CZ | PHE | B | 181 | 33.270 | 2.696 | −7.840 | 1.00 | 44.97 | 6 | C |
| ATOM | 1416 | N | VAL | B | 182 | 30.915 | 2.410 | −14.587 | 1.00 | 54.60 | 7 | N |
| ATOM | 1417 | CA | VAL | B | 182 | 30.020 | 2.724 | −15.726 | 1.00 | 58.86 | 6 | C |
| ATOM | 1418 | C | VAL | B | 182 | 29.195 | 1.478 | −16.096 | 1.00 | 61.67 | 6 | C |
| ATOM | 1419 | O | VAL | B | 182 | 27.995 | 1.643 | −16.364 | 1.00 | 61.18 | 8 | O |
| ATOM | 1420 | CB | VAL | B | 182 | 30.727 | 3.292 | −16.950 | 1.00 | 59.48 | 6 | C |
| ATOM | 1421 | CG1 | VAL | B | 182 | 29.693 | 3.662 | −18.050 | 1.00 | 59.92 | 6 | C |
| ATOM | 1422 | CG2 | VAL | B | 182 | 31.546 | 4.513 | −16.641 | 1.00 | 58.92 | 6 | C |
| ATOM | 1423 | N | SER | B | 183 | 29.792 | 0.274 | −16.095 | 1.00 | 62.99 | 7 | N |
| ATOM | 1424 | CA | SER | B | 183 | 29.000 | −0.884 | −16.487 | 1.00 | 64.63 | 6 | C |
| ATOM | 1425 | C | SER | B | 183 | 28.555 | −1.748 | −15.336 | 1.00 | 66.35 | 6 | C |
| ATOM | 1426 | O | SER | B | 183 | 28.003 | −2.834 | −15.571 | 1.00 | 68.41 | 8 | O |
| ATOM | 1427 | CB | SER | B | 183 | 29.717 | −1.718 | −17.551 | 1.00 | 66.30 | 6 | C |
| ATOM | 1428 | OG | SER | B | 183 | 29.754 | −0.980 | −18.779 | 1.00 | 67.37 | 8 | O |
| ATOM | 1429 | N | LYS | B | 184 | 28.729 | −1.328 | −14.084 | 1.00 | 66.29 | 7 | N |
| ATOM | 1430 | CA | LYS | B | 184 | 28.219 | −2.056 | −12.899 | 1.00 | 62.80 | 6 | C |
| ATOM | 1431 | C | LYS | B | 184 | 26.720 | −2.304 | −13.053 | 1.00 | 59.93 | 6 | C |
| ATOM | 1432 | O | LYS | B | 184 | 25.953 | −1.424 | −13.371 | 1.00 | 59.75 | 8 | O |
| ATOM | 1433 | CB | LYS | B | 184 | 28.446 | −1.222 | −11.642 | 1.00 | 61.88 | 6 | C |
| ATOM | 1434 | CG | LYS | B | 184 | 29.656 | −1.020 | −10.945 | 0.00 | 25.00 | 6 | C |
| ATOM | 1435 | CD | LYS | B | 184 | 29.823 | −2.244 | −10.047 | 0.00 | 25.00 | 6 | C |
| ATOM | 1436 | CE | LYS | B | 184 | 31.200 | −2.222 | −9.369 | 0.00 | 25.00 | 6 | C |
| ATOM | 1437 | NZ | LYS | B | 184 | 31.563 | −3.640 | −9.110 | 0.00 | 25.00 | 7 | N |
| ATOM | 1438 | N | PRO | B | 185 | 26.222 | −3.520 | −12.897 | 1.00 | 60.33 | 7 | N |
| ATOM | 1439 | CA | PRO | B | 185 | 24.826 | −3.856 | −13.073 | 1.00 | 59.56 | 6 | C |
| ATOM | 1440 | C | PRO | B | 185 | 23.857 | −3.053 | −12.209 | 1.00 | 59.48 | 6 | C |
| ATOM | 1441 | O | PRO | B | 185 | 22.868 | −2.443 | −12.647 | 1.00 | 59.90 | 8 | O |
| ATOM | 1442 | CB | PRO | B | 185 | 24.723 | −5.350 | −12.744 | 1.00 | 57.96 | 6 | C |
| ATOM | 1443 | CG | PRO | B | 185 | 26.107 | −5.834 | −12.584 | 1.00 | 58.07 | 6 | C |
| ATOM | 1444 | CD | PRO | B | 185 | 27.069 | −4.705 | −12.516 | 1.00 | 58.46 | 6 | C |
| ATOM | 1445 | N | GLU | B | 186 | 24.114 | −2.954 | −10.928 | 1.00 | 57.94 | 7 | N |
| ATOM | 1446 | CA | GLU | B | 186 | 23.426 | −2.261 | −9.877 | 1.00 | 57.15 | 6 | C |
| ATOM | 1447 | C | GLU | B | 186 | 22.988 | −0.851 | −10.212 | 1.00 | 55.79 | 6 | C |
| ATOM | 1448 | O | GLU | B | 186 | 21.955 | −0.331 | −9.744 | 1.00 | 54.82 | 8 | O |
| ATOM | 1449 | CB | GLU | B | 186 | 24.348 | −2.336 | −8.630 | 1.00 | 58.99 | 6 | C |
| ATOM | 1450 | CG | GLU | B | 186 | 25.747 | −2.815 | −9.021 | 1.00 | 63.74 | 6 | C |
| ATOM | 1451 | CD | GLU | B | 186 | 26.619 | −3.383 | −7.942 | 1.00 | 66.02 | 6 | C |
| ATOM | 1452 | OE1 | GLU | B | 186 | 26.369 | −3.147 | −6.737 | 1.00 | 67.71 | 8 | O |
| ATOM | 1453 | OE2 | GLU | B | 186 | 27.610 | −4.105 | −8.200 | 1.00 | 68.32 | 8 | O |
| ATOM | 1454 | N | PHE | B | 187 | 23.654 | −0.125 | −11.085 | 1.00 | 53.74 | 7 | N |
| ATOM | 1455 | CA | PHE | B | 187 | 23.308 | 1.258 | −11.410 | 1.00 | 54.01 | 6 | C |
| ATOM | 1456 | C | PHE | B | 187 | 22.751 | 1.413 | −12.804 | 1.00 | 58.29 | 6 | C |
| ATOM | 1457 | O | PHE | B | 187 | 22.825 | 2.457 | −13.454 | 1.00 | 57.88 | 8 | O |
| ATOM | 1458 | CB | PHE | B | 187 | 24.630 | 2.076 | −11.275 | 1.00 | 46.99 | 6 | C |
| ATOM | 1459 | CG | PHE | B | 187 | 25.285 | 1.983 | −9.910 | 1.00 | 39.17 | 6 | C |
| ATOM | 1460 | CD1 | PHE | B | 187 | 26.618 | 1.771 | −9.807 | 1.00 | 40.00 | 6 | C |
| ATOM | 1461 | CD2 | PHE | B | 187 | 24.589 | 2.098 | −8.753 | 1.00 | 34.80 | 6 | C |
| ATOM | 1462 | CE1 | PHE | B | 187 | 27.325 | 1.667 | −8.612 | 1.00 | 38.87 | 6 | C |
| ATOM | 1463 | CE2 | PHE | B | 187 | 25.174 | 2.010 | −7.542 | 1.00 | 37.00 | 6 | C |
| ATOM | 1464 | CZ | PHE | B | 187 | 26.554 | 1.793 | −7.458 | 1.00 | 40.19 | 6 | C |
| ATOM | 1465 | N | GLN | B | 188 | 22.146 | 0.309 | −13.283 | 1.00 | 63.83 | 7 | N |
| ATOM | 1466 | CA | GLN | B | 188 | 21.752 | 0.429 | −14.712 | 1.00 | 66.35 | 6 | C |
| ATOM | 1467 | C | GLN | B | 188 | 20.410 | 0.985 | −15.032 | 1.00 | 66.04 | 6 | C |
| ATOM | 1468 | O | GLN | B | 188 | 20.089 | 1.394 | −16.157 | 1.00 | 66.83 | 8 | O |
| ATOM | 1469 | CB | GLN | B | 188 | 22.189 | −0.895 | −15.350 | 1.00 | 68.62 | 6 | C |
| ATOM | 1470 | CG | GLN | B | 188 | 23.689 | −1.067 | −15.148 | 1.00 | 71.58 | 6 | C |
| ATOM | 1471 | CD | GLN | B | 188 | 24.629 | −0.144 | −15.882 | 1.00 | 73.82 | 6 | C |
| ATOM | 1472 | OE1 | GLN | B | 188 | 24.350 | 0.299 | −17.013 | 1.00 | 76.62 | 8 | O |
| ATOM | 1473 | NE2 | GLN | B | 188 | 25.780 | 0.184 | −15.283 | 1.00 | 73.31 | 7 | N |
| ATOM | 1474 | N | ASP | B | 189 | 19.596 | 1.134 | −13.979 | 1.00 | 64.23 | 7 | N |
| ATOM | 1475 | CA | ASP | B | 189 | 18.262 | 1.729 | −14.206 | 1.00 | 62.39 | 6 | C |
| ATOM | 1476 | C | ASP | B | 189 | 18.381 | 3.237 | −14.006 | 1.00 | 60.70 | 6 | C |
| ATOM | 1477 | O | ASP | B | 189 | 17.425 | 3.992 | −13.974 | 1.00 | 61.73 | 8 | O |
| ATOM | 1478 | CB | ASP | B | 189 | 17.240 | 0.990 | −13.530 | 0.00 | 25.00 | 6 | C |
| ATOM | 1479 | CG | ASP | B | 189 | 17.091 | −0.442 | −14.024 | 0.00 | 25.00 | 6 | C |
| ATOM | 1480 | OD1 | ASP | B | 189 | 17.760 | −0.849 | −15.007 | 0.00 | 25.00 | 8 | O |
| ATOM | 1481 | OD2 | ASP | B | 189 | 16.276 | −1.163 | −13.397 | 0.00 | 25.00 | 8 | O |
| ATOM | 1482 | N | PHE | B | 190 | 19.581 | 3.760 | −13.833 | 1.00 | 60.13 | 7 | N |
| ATOM | 1483 | CA | PHE | B | 190 | 19.906 | 5.158 | −13.737 | 1.00 | 56.59 | 6 | C |
| ATOM | 1484 | C | PHE | B | 190 | 20.248 | 5.692 | −15.148 | 1.00 | 53.53 | 6 | C |
| ATOM | 1485 | O | PHE | B | 190 | 20.494 | 4.896 | −16.046 | 1.00 | 53.48 | 8 | O |
| ATOM | 1486 | CB | PHE | B | 190 | 21.178 | 5.393 | −12.912 | 1.00 | 54.25 | 6 | C |
| ATOM | 1487 | CG | PHE | B | 190 | 20.925 | 5.180 | −11.448 | 1.00 | 54.63 | 6 | C |
| ATOM | 1488 | CD1 | PHE | B | 190 | 21.227 | 3.954 | −10.881 | 1.00 | 54.20 | 6 | C |
| ATOM | 1489 | CD2 | PHE | B | 190 | 20.397 | 6.152 | −10.607 | 1.00 | 52.87 | 6 | C |
| ATOM | 1490 | CE1 | PHE | B | 190 | 21.055 | 3.604 | −9.573 | 1.00 | 50.19 | 6 | C |
| ATOM | 1491 | CE2 | PHE | B | 190 | 20.207 | 5.851 | −9.288 | 1.00 | 51.39 | 6 | C |
| ATOM | 1492 | CZ | PHE | B | 190 | 20.523 | 4.617 | −8.779 | 1.00 | 52.70 | 6 | C |
| ATOM | 1493 | N | ASN | B | 191 | 20.291 | 7.002 | −15.240 | 1.00 | 49.84 | 7 | N |
| ATOM | 1494 | CA | ASN | B | 191 | 20.726 | 7.692 | −16.419 | 1.00 | 48.81 | 6 | C |
| ATOM | 1495 | C | ASN | B | 191 | 22.234 | 7.862 | −16.266 | 1.00 | 48.38 | 6 | C |
| ATOM | 1496 | O | ASN | B | 191 | 22.792 | 8.893 | −15.915 | 1.00 | 50.30 | 8 | O |
| ATOM | 1497 | CB | ASN | B | 191 | 20.022 | 9.042 | −16.507 | 1.00 | 51.57 | 6 | C |
| ATOM | 1498 | CG | ASN | B | 191 | 20.431 | 9.757 | −17.791 | 1.00 | 53.47 | 6 | C |
| ATOM | 1499 | OD1 | ASN | B | 191 | 20.972 | 9.056 | −18.638 | 1.00 | 56.99 | 8 | O |
| ATOM | 1500 | ND2 | ASN | B | 191 | 20.226 | 11.056 | −18.004 | 1.00 | 54.56 | 7 | N |
| ATOM | 1501 | N | ILE | B | 192 | 23.059 | 6.843 | −16.427 | 1.00 | 49.43 | 7 | N |
| ATOM | 1502 | CA | ILE | B | 192 | 24.488 | 6.954 | −16.317 | 1.00 | 48.17 | 6 | C |
| ATOM | 1503 | C | ILE | B | 192 | 24.824 | 8.115 | −17.243 | 1.00 | 50.31 | 6 | C |
| ATOM | 1504 | O | ILE | B | 192 | 24.158 | 8.480 | −18.218 | 1.00 | 48.57 | 8 | O |
| ATOM | 1505 | CB | ILE | B | 192 | 25.159 | 5.645 | −16.646 | 1.00 | 48.06 | 6 | C |
| ATOM | 1506 | CG1 | ILE | B | 192 | 24.898 | 4.596 | −15.525 | 1.00 | 46.61 | 6 | C |
| ATOM | 1507 | CG2 | ILE | B | 192 | 26.636 | 5.910 | −16.909 | 1.00 | 47.40 | 6 | C |
| ATOM | 1508 | CD1 | ILE | B | 192 | 25.321 | 3.196 | −15.975 | 1.00 | 47.78 | 6 | C |
| ATOM | 1509 | N | ILE | B | 193 | 25.796 | 8.869 | −16.819 | 1.00 | 53.89 | 7 | N |
| ATOM | 1510 | CA | ILE | B | 193 | 26.445 | 10.029 | −17.356 | 1.00 | 54.78 | 6 | C |
| ATOM | 1511 | C | ILE | B | 193 | 27.879 | 9.512 | −17.210 | 1.00 | 59.31 | 6 | C |
| ATOM | 1512 | O | ILE | B | 193 | 28.555 | 8.796 | −16.468 | 1.00 | 59.52 | 8 | O |
| ATOM | 1513 | CB | ILE | B | 193 | 26.422 | 11.344 | −16.638 | 1.00 | 54.82 | 6 | C |
| ATOM | 1514 | CG1 | ILE | B | 193 | 24.952 | 11.507 | −16.173 | 1.00 | 55.15 | 6 | C |
| ATOM | 1515 | CG2 | ILE | B | 193 | 26.845 | 12.557 | −17.438 | 1.00 | 53.20 | 6 | C |
| ATOM | 1516 | CD1 | ILE | B | 193 | 23.957 | 12.086 | −17.099 | 1.00 | 54.70 | 6 | C |
| ATOM | 1517 | N | GLU | B | 194 | 28.448 | 10.039 | −18.210 | 1.00 | 63.65 | 7 | N |
| ATOM | 1518 | CA | GLU | B | 194 | 29.778 | 9.678 | −18.611 | 1.00 | 67.51 | 6 | C |
| ATOM | 1519 | C | GLU | B | 194 | 30.897 | 10.573 | −18.286 | 1.00 | 65.79 | 6 | C |
| ATOM | 1520 | O | GLU | B | 194 | 30.885 | 11.777 | −18.519 | 1.00 | 70.27 | 8 | O |
| ATOM | 1521 | CB | GLU | B | 194 | 29.500 | 9.658 | −20.098 | 1.00 | 73.09 | 6 | C |
| ATOM | 1522 | CG | GLU | B | 194 | 28.357 | 8.685 | −20.433 | 1.00 | 79.56 | 6 | C |
| ATOM | 1523 | CD | GLU | B | 194 | 28.117 | 8.582 | −21.935 | 1.00 | 83.34 | 6 | C |
| ATOM | 1524 | OE1 | GLU | B | 194 | 28.666 | 9.426 | −22.708 | 1.00 | 84.18 | 8 | O |
| ATOM | 1525 | OE2 | GLU | B | 194 | 27.370 | 7.644 | −22.347 | 1.00 | 84.63 | 8 | O |
| ATOM | 1526 | N | ILE | B | 195 | 31.816 | 9.871 | −17.643 | 1.00 | 61.84 | 7 | N |
| ATOM | 1527 | CA | ILE | B | 195 | 32.965 | 10.691 | −17.304 | 1.00 | 61.75 | 6 | C |
| ATOM | 1528 | C | ILE | B | 195 | 34.114 | 10.088 | −18.131 | 1.00 | 62.34 | 6 | C |
| ATOM | 1529 | O | ILE | B | 195 | 34.588 | 8.979 | −17.839 | 1.00 | 61.32 | 8 | O |
| ATOM | 1530 | CB | ILE | B | 195 | 33.082 | 10.868 | −15.813 | 1.00 | 60.24 | 6 | C |
| ATOM | 1531 | CG1 | ILE | B | 195 | 31.803 | 11.382 | −15.151 | 1.00 | 58.99 | 6 | C |
| ATOM | 1532 | CG2 | ILE | B | 195 | 34.223 | 11.836 | −15.518 | 1.00 | 59.34 | 6 | C |
| ATOM | 1533 | CD1 | ILE | B | 195 | 31.138 | 12.639 | −15.670 | 1.00 | 56.46 | 6 | C |
| ATOM | 1534 | N | PRO | B | 196 | 34.464 | 10.915 | −19.118 | 1.00 | 59.41 | 7 | N |
| ATOM | 1535 | CA | PRO | B | 196 | 35.548 | 10.578 | −19.975 | 1.00 | 60.38 | 6 | C |
| ATOM | 1536 | C | PRO | B | 196 | 36.765 | 10.373 | −19.094 | 1.00 | 62.48 | 6 | C |
| ATOM | 1537 | O | PRO | B | 196 | 37.260 | 11.307 | −18.467 | 1.00 | 64.80 | 8 | O |
| ATOM | 1538 | CB | PRO | B | 196 | 35.762 | 11.788 | −20.850 | 1.00 | 61.13 | 6 | C |
| ATOM | 1539 | CG | PRO | B | 196 | 34.502 | 12.572 | −20.782 | 1.00 | 62.01 | 6 | C |
| ATOM | 1540 | CD | PRO | B | 196 | 33.910 | 12.256 | −19.417 | 1.00 | 59.45 | 6 | C |
| ATOM | 1541 | N | GLU | B | 197 | 37.272 | 9.163 | −19.082 | 1.00 | 62.41 | 7 | N |
| ATOM | 1542 | CA | GLU | B | 197 | 38.408 | 8.765 | −18.326 | 1.00 | 61.74 | 6 | C |
| ATOM | 1543 | C | GLU | B | 197 | 39.369 | 9.830 | −17.851 | 1.00 | 62.39 | 6 | C |
| ATOM | 1544 | O | GLU | B | 197 | 40.019 | 9.693 | −16.776 | 1.00 | 66.01 | 8 | O |
| ATOM | 1545 | CB | GLU | B | 197 | 39.232 | 7.731 | −19.153 | 1.00 | 61.07 | 6 | C |
| ATOM | 1546 | CG | GLU | B | 197 | 38.416 | 6.432 | −19.575 | 0.00 | 25.00 | 6 | C |
| ATOM | 1547 | CD | GLU | B | 197 | 39.311 | 5.409 | −20.239 | 0.00 | 25.00 | 6 | C |
| ATOM | 1548 | OE1 | GLU | B | 197 | 40.386 | 5.801 | −20.740 | 0.00 | 25.00 | 8 | O |
| ATOM | 1549 | OE2 | GLU | B | 197 | 38.939 | 4.218 | −20.257 | 0.00 | 25.00 | 8 | O |
| ATOM | 1550 | N | GLU | B | 198 | 39.667 | 10.910 | −18.552 | 1.00 | 59.96 | 7 | N |
| ATOM | 1551 | CA | GLU | B | 198 | 40.701 | 11.811 | −18.047 | 1.00 | 58.26 | 6 | C |
| ATOM | 1552 | C | GLU | B | 198 | 40.197 | 13.149 | −17.525 | 1.00 | 56.65 | 6 | C |
| ATOM | 1553 | O | GLU | B | 198 | 40.848 | 14.164 | −17.283 | 1.00 | 52.93 | 8 | O |
| ATOM | 1554 | CB | GLU | B | 198 | 41.777 | 11.895 | −19.106 | 1.00 | 59.96 | 6 | C |
| ATOM | 1555 | CG | GLU | B | 198 | 42.339 | 10.218 | −19.160 | 0.00 | 25.00 | 6 | C |
| ATOM | 1556 | CD | GLU | B | 198 | 42.778 | 9.823 | −20.544 | 0.00 | 25.00 | 6 | C |
| ATOM | 1557 | OE1 | GLU | B | 198 | 42.808 | 10.706 | −21.419 | 0.00 | 25.00 | 8 | O |
| ATOM | 1558 | OE2 | GLU | B | 198 | 43.131 | 8.640 | −20.734 | 0.00 | 25.00 | 8 | O |
| ATOM | 1559 | N | GLU | B | 199 | 38.891 | 13.080 | −17.337 | 1.00 | 53.10 | 7 | N |
| ATOM | 1560 | CA | GLU | B | 199 | 38.034 | 14.002 | −16.720 | 1.00 | 50.39 | 6 | C |
| ATOM | 1561 | C | GLU | B | 199 | 37.470 | 13.172 | −15.565 | 1.00 | 50.04 | 6 | C |
| ATOM | 1562 | O | GLU | B | 199 | 36.479 | 13.478 | −14.923 | 1.00 | 49.58 | 8 | O |
| ATOM | 1563 | CB | GLU | B | 199 | 36.982 | 14.621 | −17.635 | 1.00 | 54.37 | 6 | C |
| ATOM | 1564 | CG | GLU | B | 199 | 37.418 | 15.978 | −18.189 | 1.00 | 56.19 | 6 | C |
| ATOM | 1565 | CD | GLU | B | 199 | 36.305 | 16.836 | −18.698 | 1.00 | 60.19 | 6 | C |
| ATOM | 1566 | OE1 | GLU | B | 199 | 35.115 | 16.713 | −18.316 | 1.00 | 60.05 | 8 | O |
| ATOM | 1567 | OE2 | GLU | B | 199 | 36.447 | 17.759 | −19.550 | 1.00 | 63.53 | 8 | O |
| ATOM | 1568 | N | SER | B | 200 | 38.064 | 12.038 | −15.197 | 1.00 | 50.83 | 7 | N |
| ATOM | 1569 | CA | SER | B | 200 | 37.597 | 11.154 | −14.151 | 1.00 | 47.31 | 6 | C |
| ATOM | 1570 | C | SER | B | 200 | 37.374 | 11.877 | −12.826 | 1.00 | 46.88 | 6 | C |
| ATOM | 1571 | O | SER | B | 200 | 36.276 | 11.721 | −12.256 | 1.00 | 44.55 | 8 | O |
| ATOM | 1572 | CB | SER | B | 200 | 38.564 | 9.997 | −13.998 | 1.00 | 49.88 | 6 | C |
| ATOM | 1573 | OG | SER | B | 200 | 39.569 | 10.107 | −12.991 | 1.00 | 53.63 | 8 | O |
| ATOM | 1574 | N | TYR | B | 201 | 38.331 | 12.635 | −12.292 | 1.00 | 42.74 | 7 | N |
| ATOM | 1575 | CA | TYR | B | 201 | 38.153 | 13.381 | −11.068 | 1.00 | 40.48 | 6 | C |
| ATOM | 1576 | C | TYR | B | 201 | 36.801 | 14.072 | −11.012 | 1.00 | 42.78 | 6 | C |
| ATOM | 1577 | O | TYR | B | 201 | 36.183 | 14.146 | −9.955 | 1.00 | 41.39 | 8 | O |
| ATOM | 1578 | CB | TYR | B | 201 | 39.088 | 14.601 | −10.936 | 1.00 | 37.63 | 6 | C |
| ATOM | 1579 | CG | TYR | B | 201 | 39.167 | 15.286 | −9.611 | 1.00 | 33.60 | 6 | C |
| ATOM | 1580 | CD1 | TYR | B | 201 | 39.395 | 14.635 | −8.409 | 1.00 | 30.95 | 6 | C |
| ATOM | 1581 | CD2 | TYR | B | 201 | 39.001 | 16.700 | −9.551 | 1.00 | 30.82 | 6 | C |
| ATOM | 1582 | CE1 | TYR | B | 201 | 39.458 | 15.333 | −7.204 | 1.00 | 28.50 | 6 | C |
| ATOM | 1583 | CE2 | TYR | B | 201 | 39.065 | 17.408 | −8.383 | 1.00 | 26.77 | 6 | C |
| ATOM | 1584 | CZ | TYR | B | 201 | 39.295 | 16.724 | −7.196 | 1.00 | 26.41 | 6 | C |
| ATOM | 1585 | OH | TYR | B | 201 | 39.350 | 17.446 | −6.030 | 1.00 | 24.65 | 8 | O |
| ATOM | 1586 | N | ALA | B | 202 | 36.260 | 14.605 | −12.121 | 1.00 | 44.21 | 7 | N |
| ATOM | 1587 | CA | ALA | B | 202 | 34.976 | 15.261 | −12.166 | 1.00 | 42.10 | 6 | C |
| ATOM | 1588 | C | ALA | B | 202 | 33.831 | 14.329 | −11.907 | 1.00 | 43.50 | 6 | C |
| ATOM | 1589 | O | ALA | B | 202 | 32.625 | 14.698 | −11.928 | 1.00 | 47.54 | 8 | O |
| ATOM | 1590 | CB | ALA | B | 202 | 34.688 | 15.937 | −13.489 | 1.00 | 42.96 | 6 | C |
| ATOM | 1591 | N | ALA | B | 203 | 34.051 | 13.068 | −11.588 | 1.00 | 43.06 | 7 | N |
| ATOM | 1592 | CA | ALA | B | 203 | 32.902 | 12.216 | −11.268 | 1.00 | 42.92 | 6 | C |
| ATOM | 1593 | C | ALA | B | 203 | 32.482 | 12.527 | −9.830 | 1.00 | 41.28 | 6 | C |
| ATOM | 1594 | O | ALA | B | 203 | 31.483 | 11.996 | −9.333 | 1.00 | 42.49 | 8 | O |
| ATOM | 1595 | CB | ALA | B | 203 | 33.188 | 10.712 | −11.423 | 1.00 | 42.56 | 6 | C |
| ATOM | 1596 | N | ASN | B | 204 | 33.238 | 13.277 | −9.061 | 1.00 | 37.98 | 7 | N |
| ATOM | 1597 | CA | ASN | B | 204 | 32.886 | 13.645 | −7.684 | 1.00 | 35.34 | 6 | C |
| ATOM | 1598 | C | ASN | B | 204 | 32.407 | 15.101 | −7.790 | 1.00 | 34.62 | 6 | C |
| ATOM | 1599 | O | ASN | B | 204 | 33.148 | 16.070 | −8.013 | 1.00 | 34.32 | 8 | O |
| ATOM | 1600 | CB | ASN | B | 204 | 34.033 | 13.344 | −6.753 | 1.00 | 35.46 | 6 | C |
| ATOM | 1601 | CG | ASN | B | 204 | 34.044 | 14.083 | −5.463 | 1.00 | 38.66 | 6 | C |
| ATOM | 1602 | OD1 | ASN | B | 204 | 33.091 | 14.714 | −5.040 | 1.00 | 38.32 | 8 | O |
| ATOM | 1603 | ND2 | ASN | B | 204 | 35.173 | 14.050 | −4.738 | 1.00 | 41.78 | 7 | N |
| ATOM | 1604 | N | CYS | B | 205 | 31.099 | 15.227 | −7.716 | 1.00 | 27.97 | 7 | N |
| ATOM | 1605 | CA | CYS | B | 205 | 30.402 | 16.460 | −7.831 | 1.00 | 29.14 | 6 | C |
| ATOM | 1606 | C | CYS | B | 205 | 29.297 | 16.419 | −6.769 | 1.00 | 28.62 | 6 | C |
| ATOM | 1607 | O | CYS | B | 205 | 29.025 | 15.319 | −6.420 | 1.00 | 28.89 | 8 | O |
| ATOM | 1608 | CB | CYS | B | 205 | 29.678 | 16.605 | −9.204 | 1.00 | 26.33 | 6 | C |
| ATOM | 1609 | SG | CYS | B | 205 | 30.947 | 16.933 | −10.481 | 1.00 | 35.46 | 16 | S |
| ATOM | 1610 | N | ILE | B | 206 | 28.641 | 17.504 | −6.350 | 1.00 | 26.31 | 7 | N |
| ATOM | 1611 | CA | ILE | B | 206 | 27.575 | 17.312 | −5.431 | 1.00 | 22.89 | 6 | C |
| ATOM | 1612 | C | ILE | B | 206 | 26.420 | 18.180 | −5.863 | 1.00 | 23.42 | 6 | C |
| ATOM | 1613 | O | ILE | B | 206 | 26.466 | 19.338 | −6.337 | 1.00 | 26.59 | 8 | O |
| ATOM | 1614 | CB | ILE | B | 206 | 27.918 | 17.743 | −4.002 | 1.00 | 27.58 | 6 | C |
| ATOM | 1615 | CG1 | ILE | B | 206 | 28.728 | 19.059 | −4.027 | 1.00 | 27.92 | 6 | C |
| ATOM | 1616 | CG2 | ILE | B | 206 | 28.716 | 16.735 | −3.216 | 1.00 | 28.55 | 6 | C |
| ATOM | 1617 | CD1 | ILE | B | 206 | 28.658 | 19.887 | −2.826 | 1.00 | 28.37 | 6 | C |
| ATOM | 1618 | N | TRP | B | 207 | 25.287 | 17.567 | −5.660 | 1.00 | 20.80 | 7 | N |
| ATOM | 1619 | CA | TRP | B | 207 | 23.974 | 18.121 | −5.899 | 1.00 | 21.62 | 6 | C |
| ATOM | 1620 | C | TRP | B | 207 | 23.600 | 18.855 | −4.647 | 1.00 | 25.93 | 6 | C |
| ATOM | 1621 | O | TRP | B | 207 | 23.653 | 18.328 | −3.487 | 1.00 | 26.88 | 8 | O |
| ATOM | 1622 | CB | TRP | B | 207 | 22.962 | 17.076 | −6.317 | 1.00 | 23.19 | 6 | C |
| ATOM | 1623 | CG | TRP | B | 207 | 21.572 | 17.619 | −6.386 | 1.00 | 27.81 | 6 | C |
| ATOM | 1624 | CD1 | TRP | B | 207 | 20.981 | 18.049 | −7.520 | 1.00 | 28.98 | 6 | C |
| ATOM | 1625 | CD2 | TRP | B | 207 | 20.594 | 17.804 | −5.320 | 1.00 | 28.79 | 6 | C |
| ATOM | 1626 | NE1 | TRP | B | 207 | 19.688 | 18.497 | −7.250 | 1.00 | 31.38 | 7 | N |
| ATOM | 1627 | CE2 | TRP | B | 207 | 19.437 | 18.352 | −5.891 | 1.00 | 28.76 | 6 | C |
| ATOM | 1628 | CE3 | TRP | B | 207 | 20.613 | 17.551 | −3.935 | 1.00 | 27.53 | 6 | C |
| ATOM | 1629 | CZ2 | TRP | B | 207 | 18.289 | 18.668 | −5.183 | 1.00 | 24.45 | 6 | C |
| ATOM | 1630 | CZ3 | TRP | B | 207 | 19.475 | 17.852 | −3.190 | 1.00 | 29.18 | 6 | C |
| ATOM | 1631 | CH2 | TRP | B | 207 | 18.310 | 18.414 | −3.826 | 1.00 | 25.51 | 6 | C |
| ATOM | 1632 | N | VAL | B | 208 | 23.224 | 20.150 | −4.755 | 1.00 | 27.07 | 7 | N |
| ATOM | 1633 | CA | VAL | B | 208 | 22.807 | 20.821 | −3.538 | 1.00 | 25.26 | 6 | C |
| ATOM | 1634 | C | VAL | B | 208 | 21.586 | 21.661 | −3.853 | 1.00 | 27.05 | 6 | C |
| ATOM | 1635 | O | VAL | B | 208 | 21.781 | 22.783 | −4.266 | 1.00 | 26.98 | 8 | O |
| ATOM | 1636 | CB | VAL | B | 208 | 23.828 | 21.736 | −2.785 | 1.00 | 25.03 | 6 | C |
| ATOM | 1637 | CG1 | VAL | B | 208 | 23.144 | 22.221 | −1.467 | 1.00 | 20.94 | 6 | C |
| ATOM | 1638 | CG2 | VAL | B | 208 | 25.098 | 21.016 | −2.445 | 1.00 | 20.89 | 6 | C |
| ATOM | 1639 | N | ASN | B | 209 | 20.359 | 21.175 | −3.650 | 1.00 | 27.42 | 7 | N |
| ATOM | 1640 | CA | ASN | B | 209 | 19.223 | 22.022 | −3.881 | 1.00 | 27.77 | 6 | C |
| ATOM | 1641 | C | ASN | B | 209 | 19.167 | 22.536 | −5.298 | 1.00 | 29.35 | 6 | C |
| ATOM | 1642 | O | ASN | B | 209 | 18.687 | 23.650 | −5.521 | 1.00 | 31.67 | 8 | O |
| ATOM | 1643 | CB | ASN | B | 209 | 18.950 | 23.149 | −2.830 | 1.00 | 28.45 | 6 | C |
| ATOM | 1644 | CG | ASN | B | 209 | 18.980 | 22.659 | −1.415 | 1.00 | 29.44 | 6 | C |
| ATOM | 1645 | OD1 | ASN | B | 209 | 18.763 | 21.448 | −1.097 | 1.00 | 30.40 | 8 | O |
| ATOM | 1646 | ND2 | ASN | B | 209 | 19.253 | 23.531 | −0.471 | 1.00 | 30.16 | 7 | N |
| ATOM | 1647 | N | GLU | B | 210 | 19.513 | 21.707 | −6.281 | 1.00 | 33.04 | 7 | N |
| ATOM | 1648 | CA | GLU | B | 210 | 19.375 | 21.974 | −7.700 | 1.00 | 36.06 | 6 | C |
| ATOM | 1649 | C | GLU | B | 210 | 20.503 | 22.731 | −8.348 | 1.00 | 38.43 | 6 | C |
| ATOM | 1650 | O | GLU | B | 210 | 20.401 | 23.002 | −9.537 | 1.00 | 40.10 | 8 | O |
| ATOM | 1651 | CB | GLU | B | 210 | 18.000 | 22.497 | −8.079 | 1.00 | 35.70 | 6 | C |
| ATOM | 1652 | CG | GLU | B | 210 | 16.819 | 21.674 | −7.706 | 1.00 | 35.27 | 6 | C |
| ATOM | 1653 | CD | GLU | B | 210 | 16.711 | 20.214 | −8.012 | 1.00 | 41.37 | 6 | C |
| ATOM | 1654 | OE1 | GLU | B | 210 | 17.551 | 19.438 | −8.499 | 1.00 | 44.61 | 8 | O |
| ATOM | 1655 | OE2 | GLU | B | 210 | 15.589 | 19.651 | −7.739 | 1.00 | 46.00 | 8 | O |
| ATOM | 1656 | N | ARG | B | 211 | 21.593 | 22.959 | −7.607 | 1.00 | 37.25 | 7 | N |
| ATOM | 1657 | CA | ARG | B | 211 | 22.788 | 23.491 | −8.218 | 1.00 | 35.35 | 6 | C |
| ATOM | 1658 | C | ARG | B | 211 | 23.671 | 22.249 | −8.049 | 1.00 | 36.22 | 6 | C |
| ATOM | 1659 | O | ARG | B | 211 | 23.314 | 21.298 | −7.283 | 1.00 | 33.62 | 8 | O |
| ATOM | 1660 | CB | ARG | B | 211 | 23.417 | 24.656 | −7.487 | 1.00 | 37.24 | 6 | C |
| ATOM | 1661 | CG | ARG | B | 211 | 22.669 | 25.189 | −6.335 | 1.00 | 33.96 | 6 | C |
| ATOM | 1662 | CD | ARG | B | 211 | 21.931 | 26.453 | −6.516 | 1.00 | 36.55 | 6 | C |
| ATOM | 1663 | NE | ARG | B | 211 | 20.769 | 26.650 | −5.666 | 1.00 | 33.87 | 7 | N |
| ATOM | 1664 | CZ | ARG | B | 211 | 19.877 | 27.198 | −4.956 | 1.00 | 33.75 | 6 | C |
| ATOM | 1665 | NH1 | ARG | B | 211 | 19.594 | 28.433 | −4.572 | 1.00 | 33.90 | 7 | N |
| ATOM | 1666 | MH2 | ARG | B | 211 | 18.921 | 26.390 | −4.403 | 1.00 | 34.54 | 7 | N |
| ATOM | 1667 | N | VAL | B | 212 | 24.840 | 22.276 | −8.729 | 1.00 | 33.29 | 7 | N |
| ATOM | 1668 | CA | VAL | B | 212 | 25.764 | 21.184 | −8.641 | 1.00 | 25.67 | 6 | C |
| ATOM | 1669 | C | VAL | B | 212 | 27.160 | 21.833 | −8.622 | 1.00 | 27.37 | 6 | C |
| ATOM | 1670 | O | VAL | B | 212 | 27.548 | 22.580 | −9.546 | 1.00 | 25.09 | 8 | O |
| ATOM | 1671 | CB | VAL | B | 212 | 25.662 | 20.246 | −9.855 | 1.00 | 25.34 | 6 | C |
| ATOM | 1672 | CG1 | VAL | B | 212 | 26.625 | 19.055 | −9.737 | 1.00 | 22.91 | 6 | C |
| ATOM | 1673 | CG2 | VAL | B | 212 | 24.298 | 19.689 | −10.090 | 1.00 | 25.28 | 6 | C |
| ATOM | 1674 | N | ILE | B | 213 | 27.903 | 21.514 | −7.523 | 1.00 | 26.12 | 7 | N |
| ATOM | 1675 | CA | ILE | B | 213 | 29.243 | 22.052 | −7.397 | 1.00 | 24.04 | 6 | C |
| ATOM | 1676 | C | ILE | B | 213 | 30.170 | 20.987 | −7.933 | 1.00 | 26.98 | 6 | C |
| ATOM | 1677 | O | ILE | B | 213 | 30.113 | 19.868 | −7.444 | 1.00 | 29.90 | 8 | O |
| ATOM | 1678 | CB | ILE | B | 213 | 29.506 | 22.432 | −5.950 | 1.00 | 25.10 | 6 | C |
| ATOM | 1679 | CG1 | ILE | B | 213 | 28.393 | 23.410 | −5.516 | 1.00 | 23.98 | 6 | C |
| ATOM | 1680 | CG2 | ILE | B | 213 | 30.861 | 23.078 | −5.575 | 1.00 | 21.32 | 6 | C |
| ATOM | 1681 | CD1 | ILE | B | 213 | 28.353 | 23.508 | −3.997 | 1.00 | 26.97 | 6 | C |
| ATOM | 1682 | N | MET | B | 214 | 30.985 | 21.315 | −8.963 | 1.00 | 28.18 | 7 | N |
| ATOM | 1683 | CA | MET | B | 214 | 31.926 | 20.356 | −9.544 | 1.00 | 28.81 | 6 | C |
| ATOM | 1684 | C | MET | B | 214 | 33.304 | 20.986 | −9.447 | 1.00 | 28.06 | 6 | C |
| ATOM | 1685 | O | MET | B | 214 | 33.431 | 22.211 | −9.331 | 1.00 | 26.98 | 8 | O |
| ATOM | 1686 | CB | MET | B | 214 | 31.301 | 20.225 | −10.945 | 1.00 | 28.22 | 6 | C |
| ATOM | 1687 | CG | MET | B | 214 | 31.695 | 21.335 | −11.926 | 1.00 | 34.38 | 6 | C |
| ATOM | 1688 | SD | MET | B | 214 | 31.137 | 20.931 | −13.632 | 1.00 | 37.90 | 16 | S |
| ATOM | 1689 | CE | MET | B | 214 | 32.236 | 19.556 | −13.964 | 1.00 | 26.63 | 6 | C |
| ATOM | 1690 | N | PRO | B | 215 | 34.384 | 20.260 | −9.498 | 1.00 | 30.52 | 7 | N |
| ATOM | 1691 | CA | PRO | B | 215 | 35.749 | 20.856 | −9.538 | 1.00 | 31.42 | 6 | C |
| ATOM | 1692 | C | PRO | B | 215 | 35.814 | 21.645 | −10.844 | 1.00 | 34.04 | 6 | C |
| ATOM | 1693 | O | PRO | B | 215 | 35.038 | 21.210 | −11.701 | 1.00 | 33.76 | 8 | O |
| ATOM | 1694 | CB | PRO | B | 215 | 36.674 | 19.692 | −9.803 | 1.00 | 25.81 | 6 | C |
| ATOM | 1695 | CG | PRO | B | 215 | 35.824 | 18.557 | −10.240 | 1.00 | 24.44 | 6 | C |
| ATOM | 1696 | CD | PRO | B | 215 | 34.429 | 18.788 | −9.682 | 1.00 | 27.57 | 6 | C |
| ATOM | 1697 | N | ALA | B | 216 | 36.608 | 22.707 | −10.872 | 1.00 | 37.28 | 7 | N |
| ATOM | 1698 | CA | ALA | B | 216 | 36.711 | 23.420 | −12.165 | 1.00 | 39.55 | 6 | C |
| ATOM | 1699 | C | ALA | B | 216 | 37.763 | 22.787 | −13.054 | 1.00 | 38.16 | 6 | C |
| ATOM | 1700 | O | ALA | B | 216 | 38.692 | 22.179 | −12.550 | 1.00 | 39.24 | 8 | O |
| ATOM | 1701 | CB | ALA | B | 216 | 37.180 | 24.860 | −11.924 | 1.00 | 35.38 | 6 | C |
| ATOM | 1702 | N | GLY | B | 217 | 37.763 | 23.908 | −14.358 | 1.00 | 43.03 | 7 | N |
| ATOM | 1703 | CA | GLY | B | 217 | 38.772 | 22.480 | −15.280 | 1.00 | 42.21 | 6 | O |
| ATOM | 1704 | C | GLY | B | 217 | 38.595 | 21.134 | −15.920 | 1.00 | 40.83 | 6 | C |
| ATOM | 1705 | O | GLY | B | 217 | 39.502 | 20.412 | −16.358 | 1.00 | 47.08 | 8 | O |
| ATOM | 1706 | N | SER | B | 218 | 37.392 | 20.751 | −16.117 | 1.00 | 38.83 | 7 | N |
| ATOM | 1707 | CA | SER | B | 218 | 36.801 | 19.543 | −16.594 | 1.00 | 41.37 | 6 | C |
| ATOM | 1708 | C | SER | B | 218 | 35.636 | 19.972 | −17.450 | 1.00 | 42.03 | 6 | C |
| ATOM | 1709 | O | SER | B | 218 | 34.519 | 19.752 | −17.092 | 1.00 | 41.86 | 8 | O |
| ATOM | 1710 | CB | SER | B | 218 | 36.418 | 18.736 | −15.311 | 1.00 | 42.49 | 6 | C |
| ATOM | 1711 | OG | SER | B | 218 | 37.571 | 18.152 | −14.611 | 1.00 | 44.64 | 8 | O |
| ATOM | 1712 | N | PRO | B | 219 | 35.901 | 20.578 | −18.626 | 1.00 | 45.51 | 7 | N |
| ATOM | 1713 | CA | PRO | B | 219 | 34.924 | 21.059 | −19.559 | 1.00 | 44.25 | 6 | C |
| ATOM | 1714 | C | PRO | B | 219 | 33.929 | 20.055 | −20.142 | 1.00 | 47.64 | 6 | C |
| ATOM | 1715 | O | PRO | B | 219 | 32.749 | 20.409 | −20.229 | 1.00 | 46.90 | 8 | O |
| ATOM | 1716 | CB | PRO | B | 219 | 35.752 | 21.493 | −20.776 | 1.00 | 43.16 | 6 | C |
| ATOM | 1717 | CG | PRO | B | 219 | 37.160 | 21.490 | −20.362 | 1.00 | 40.58 | 6 | C |
| ATOM | 1718 | CD | PRO | B | 219 | 37.323 | 20.771 | −19.131 | 1.00 | 41.32 | 6 | C |
| ATOM | 1719 | N | ARG | B | 220 | 34.415 | 18.900 | −20.579 | 1.00 | 49.25 | 7 | N |
| ATOM | 1720 | CA | ARG | B | 220 | 33.470 | 17.932 | −21.188 | 1.00 | 53.32 | 6 | C |
| ATOM | 1721 | C | ARG | B | 220 | 32.435 | 17.755 | −20.102 | 1.00 | 54.00 | 6 | C |
| ATOM | 1722 | O | ARG | B | 220 | 31.302 | 18.279 | −20.279 | 1.00 | 54.06 | 8 | O |
| ATOM | 1723 | CB | ARG | B | 220 | 34.352 | 16.794 | −21.695 | 1.00 | 56.85 | 6 | C |
| ATOM | 1724 | CG | ARG | B | 220 | 34.927 | 17.028 | −23.088 | 1.00 | 60.15 | 6 | C |
| ATOM | 1725 | CD | ARG | B | 220 | 35.551 | 15.747 | −23.650 | 1.00 | 64.80 | 6 | C |
| ATOM | 1726 | NE | ARG | B | 220 | 34.534 | 14.675 | −23.687 | 1.00 | 68.90 | 7 | N |
| ATOM | 1727 | CZ | ARG | B | 220 | 33.652 | 14.544 | −24.682 | 1.00 | 69.79 | 6 | C |
| ATOM | 1728 | NH1 | ARG | B | 220 | 33.717 | 15.439 | −25.683 | 1.00 | 72.53 | 7 | N |
| ATOM | 1729 | NH2 | ARG | B | 220 | 32.752 | 13.573 | −24.684 | 1.00 | 68.980 | 7 | N |
| ATOM | 1730 | N | THR | B | 221 | 32.718 | 17.163 | −18.899 | 1.00 | 50.81 | 7 | N |
| ATOM | 1731 | CA | THR | B | 221 | 31.669 | 17.139 | −17.846 | 1.00 | 46.53 | 6 | C |
| ATOM | 1732 | C | THR | B | 221 | 30.840 | 18.384 | −17.484 | 1.00 | 45.24 | 6 | C |
| ATOM | 1733 | O | THR | B | 221 | 29.599 | 18.182 | −17.261 | 1.00 | 38.57 | 8 | O |
| ATOM | 1734 | CB | THR | B | 221 | 32.313 | 16.655 | −16.564 | 1.00 | 46.37 | 6 | C |
| ATOM | 1735 | OG1 | THR | B | 221 | 32.858 | 15.364 | −16.786 | 1.00 | 49.91 | 8 | O |
| ATOM | 1736 | CG2 | THR | B | 221 | 31.377 | 16.470 | −15.386 | 1.00 | 45.72 | 6 | C |
| ATOM | 1737 | N | ARG | B | 222 | 31.280 | 19.647 | −17.274 | 1.00 | 44.87 | 7 | N |
| ATOM | 1738 | CA | ARG | B | 222 | 30.351 | 20.737 | −16.974 | 1.00 | 45.37 | 6 | C |
| ATOM | 1739 | C | ARG | B | 222 | 29.242 | 20.739 | −18.033 | 1.00 | 45.10 | 6 | C |
| ATOM | 1740 | O | ARG | B | 222 | 28.123 | 21.008 | −17.589 | 1.00 | 46.01 | 8 | O |
| ATOM | 1741 | CB | ARG | B | 222 | 30.931 | 22.163 | −17.015 | 1.00 | 47.21 | 6 | C |
| ATOM | 1742 | CG | ARG | B | 222 | 29.997 | 23.276 | −16.557 | 1.00 | 51.54 | 6 | C |
| ATOM | 1743 | CD | ARG | B | 222 | 30.366 | 24.685 | −16.945 | 1.00 | 54.20 | 6 | C |
| ATOM | 1744 | NE | ARG | B | 222 | 29.218 | 25.622 | −16.913 | 1.00 | 58.11 | 7 | N |
| ATOM | 1745 | CZ | ARG | B | 222 | 28.226 | 25.618 | −17.817 | 1.00 | 59.44 | 6 | C |
| ATOM | 1746 | NH1 | ARG | B | 222 | 28.275 | 24.715 | −18.807 | 1.00 | 62.59 | 7 | N |
| ATOM | 1747 | NH2 | ARG | B | 222 | 27.209 | 26.464 | −17.773 | 1.00 | 59.29 | 7 | N |
| ATOM | 1748 | N | GLU | B | 223 | 29.511 | 20.569 | −19.334 | 1.00 | 45.24 | 7 | N |
| ATOM | 1749 | CA | GLU | B | 223 | 28.464 | 20.590 | −20.368 | 1.00 | 45.06 | 6 | C |
| ATOM | 1750 | C | GLU | B | 223 | 27.606 | 19.340 | −20.176 | 1.00 | 46.26 | 6 | C |
| ATOM | 1751 | O | GLU | B | 223 | 26.381 | 19.456 | −20.025 | 1.00 | 45.82 | 8 | O |
| ATOM | 1752 | CB | GLU | B | 223 | 29.013 | 20.663 | −21.799 | 1.00 | 48.10 | 6 | C |
| ATOM | 1753 | CG | GLU | B | 223 | 29.778 | 21.915 | −22.207 | 0.00 | 25.00 | 6 | C |
| ATOM | 1754 | CD | GLU | B | 223 | 28.909 | 23.156 | −22.143 | 0.00 | 25.00 | 6 | C |
| ATOM | 1755 | OE1 | GLU | B | 223 | 27.743 | 23.087 | −22.583 | 0.00 | 25.00 | 8 | O |
| ATOM | 1756 | OE2 | GLU | B | 223 | 29.395 | 24.196 | −21.650 | 0.00 | 25.00 | 8 | O |
| ATOM | 1757 | N | LYS | B | 224 | 28.213 | 18.125 | −20.109 | 1.00 | 47.12 | 7 | N |
| ATOM | 1758 | CA | LYS | B | 224 | 27.315 | 16.975 | −19.859 | 1.00 | 48.40 | 6 | C |
| ATOM | 1759 | C | LYS | B | 224 | 26.481 | 17.328 | −18.623 | 1.00 | 50.64 | 6 | C |
| ATOM | 1760 | O | LYS | B | 224 | 25.295 | 16.951 | −18.657 | 1.00 | 52.79 | 8 | O |
| ATOM | 1761 | CB | LYS | B | 224 | 27.965 | 15.637 | −19.788 | 1.00 | 49.30 | 6 | C |
| ATOM | 1762 | CC | LYS | B | 224 | 29.018 | 14.917 | −20.589 | 1.00 | 50.38 | 6 | C |
| ATOM | 1763 | CD | LYS | B | 224 | 29.232 | 13.311 | −20.822 | 0.00 | 25.00 | 6 | C |
| ATOM | 1764 | CE | LYS | B | 224 | 30.487 | 12.882 | −21.581 | 0.00 | 25.00 | 6 | C |
| ATOM | 1765 | NZ | LYS | B | 224 | 30.375 | 11.499 | −22.128 | 0.00 | 25.00 | 7 | N |
| ATOM | 1766 | N | ILE | B | 225 | 26.954 | 17.991 | −17.517 | 1.00 | 49.96 | 7 | N |
| ATOM | 1767 | CA | ILE | B | 225 | 26.019 | 18.313 | −16.454 | 1.00 | 47.77 | 6 | C |
| ATOM | 1768 | C | ILE | B | 225 | 25.105 | 19.500 | −16.744 | 1.00 | 46.18 | 6 | C |
| ATOM | 1769 | O | ILE | B | 225 | 23.891 | 19.435 | −16.532 | 1.00 | 45.48 | 8 | O |
| ATOM | 1770 | CB | ILE | B | 225 | 26.706 | 18.579 | −15.104 | 1.00 | 47.65 | 6 | C |
| ATOM | 1771 | CG1 | ILE | B | 225 | 27.542 | 17.361 | −14.703 | 1.00 | 47.20 | 6 | C |
| ATOM | 1772 | CG2 | ILE | B | 225 | 25.702 | 18.918 | −13.985 | 1.00 | 45.10 | 6 | C |
| ATOM | 1773 | CD1 | ILE | B | 225 | 28.377 | 17.702 | −13.458 | 1.00 | 42.77 | 6 | C |
| ATOM | 1774 | N | ALA | B | 226 | 25.534 | 20.686 | −17.142 | 1.00 | 47.70 | 7 | N |
| ATOM | 1775 | CA | ALA | B | 226 | 24.691 | 21.864 | −17.407 | 1.00 | 48.34 | 6 | C |
| ATOM | 1776 | C | ALA | B | 226 | 23.533 | 21.553 | −18.346 | 1.00 | 52.38 | 6 | C |
| ATOM | 1777 | O | ALA | B | 226 | 22.392 | 22.016 | −18.177 | 1.00 | 53.93 | 8 | O |
| ATOM | 1778 | CB | ALA | B | 226 | 25.548 | 22.989 | −17.956 | 1.00 | 49.59 | 6 | C |
| ATOM | 1779 | N | ARG | B | 227 | 23.740 | 20.746 | −19.401 | 1.00 | 53.96 | 7 | N |
| ATOM | 1780 | CA | ARG | B | 227 | 22.555 | 20.434 | −20.228 | 1.00 | 59.43 | 6 | C |
| ATOM | 1781 | C | ARG | B | 227 | 21.888 | 19.268 | −19.482 | 1.00 | 58.70 | 6 | C |
| ATOM | 1782 | O | ARG | B | 227 | 22.305 | 18.132 | −19.756 | 1.00 | 62.82 | 8 | O |
| ATOM | 1783 | CB | ARG | B | 227 | 22.872 | 20.008 | −21.665 | 1.00 | 59.79 | 6 | C |
| ATOM | 1784 | CG | ARG | B | 227 | 24.477 | 19.338 | −22.017 | 0.00 | 25.00 | 6 | C |
| ATOM | 1785 | CD | ARG | B | 227 | 24.888 | 18.211 | −22.951 | 0.00 | 25.00 | 6 | C |
| ATOM | 1786 | NE | ARG | B | 227 | 25.960 | 18.613 | −23.855 | 0.00 | 25.00 | 7 | N |
| ATOM | 1787 | CZ | ARG | B | 227 | 26.502 | 17.817 | −24.772 | 0.00 | 25.00 | 6 | C |
| ATOM | 1788 | NH1 | ARG | B | 227 | 27.034 | 17.247 | −23.996 | 1.00 | 48.61 | 7 | N |
| ATOM | 1789 | NH2 | ARG | B | 227 | 26.711 | 18.895 | −25.737 | 1.00 | 39.83 | 7 | N |
| ATOM | 1790 | N | LEU | B | 228 | 21.000 | 19.468 | −18.573 | 1.00 | 53.96 | 7 | N |
| ATOM | 1791 | CA | LEU | B | 228 | 20.289 | 18.471 | −17.766 | 1.00 | 49.26 | 6 | C |
| ATOM | 1792 | C | LEU | B | 228 | 19.443 | 19.464 | −16.911 | 1.00 | 45.31 | 6 | C |
| ATOM | 1793 | O | LEU | B | 228 | 18.514 | 19.219 | −16.209 | 1.00 | 43.53 | 8 | O |
| ATOM | 1794 | CB | LEU | B | 228 | 21.005 | 17.481 | −16.893 | 1.00 | 46.87 | 6 | C |
| ATOM | 1795 | CG | LEU | B | 228 | 21.854 | 16.320 | −17.309 | 1.00 | 48.28 | 6 | C |
| ATOM | 1796 | CD1 | LEU | B | 228 | 22.670 | 15.754 | −16.132 | 1.00 | 47.79 | 6 | C |
| ATOM | 1797 | CD2 | LEU | B | 228 | 21.072 | 15.107 | −17.844 | 1.00 | 47.43 | 6 | C |
| ATOM | 1798 | N | GLY | B | 229 | 19.822 | 20.756 | −17.007 | 1.00 | 42.40 | 7 | N |
| ATOM | 1799 | CA | GLY | B | 229 | 19.144 | 21.789 | −16.328 | 1.00 | 41.81 | 6 | C |
| ATOM | 1800 | C | GLY | B | 229 | 19.659 | 22.255 | −14.975 | 1.00 | 44.76 | 6 | C |
| ATOM | 1801 | O | GLY | B | 229 | 18.986 | 23.202 | −14.458 | 1.00 | 43.23 | 8 | O |
| ATOM | 1802 | N | TYR | B | 230 | 20.751 | 21.687 | −14.432 | 1.00 | 42.84 | 7 | N |
| ATOM | 1803 | CA | TYR | B | 230 | 21.125 | 22.245 | −13.142 | 1.00 | 42.46 | 6 | C |
| ATOM | 1804 | C | TYR | B | 230 | 22.012 | 23.482 | −13.334 | 1.00 | 43.47 | 6 | C |
| ATOM | 1805 | O | TYR | B | 230 | 22.808 | 23.628 | −14.236 | 1.00 | 44.42 | 8 | O |
| ATOM | 1806 | CB | TYR | B | 230 | 21.884 | 21.280 | −12.236 | 1.00 | 40.53 | 6 | C |
| ATOM | 1807 | CG | TYR | B | 230 | 21.402 | 19.851 | −12.062 | 1.00 | 40.23 | 6 | C |
| ATOM | 1808 | CD1 | TYR | B | 230 | 20.493 | 19.473 | −11.052 | 1.00 | 39.45 | 6 | C |
| ATOM | 1809 | CD2 | TYR | B | 230 | 21.846 | 18.857 | −12.911 | 1.00 | 38.18 | 6 | C |
| ATOM | 1810 | CE1 | TYR | B | 230 | 20.059 | 18.177 | −10.904 | 1.00 | 37.62 | 6 | C |
| ATOM | 1811 | CE2 | TYR | B | 230 | 21.442 | 17.552 | −12.798 | 1.00 | 36.99 | 6 | C |
| ATOM | 1812 | CZ | TYR | B | 230 | 20.554 | 17.225 | −11.798 | 1.00 | 37.90 | 6 | C |
| ATOM | 1813 | OH | TYR | B | 230 | 20.173 | 15.913 | −11.704 | 1.00 | 37.00 | 8 | O |
| ATOM | 1814 | N | ARG | B | 231 | 21.919 | 24.360 | −12.340 | 1.00 | 44.96 | 7 | N |
| ATOM | 1815 | CA | ARG | B | 231 | 22.808 | 25.506 | −12.284 | 1.00 | 46.21 | 6 | C |
| ATOM | 1816 | C | ARG | B | 231 | 24.154 | 24.938 | −11.835 | 1.00 | 44.69 | 6 | C |
| ATOM | 1817 | O | ARG | B | 231 | 24.126 | 24.385 | −10.726 | 1.00 | 46.00 | 8 | O |
| ATOM | 1818 | CB | ARG | B | 231 | 22.262 | 26.485 | −11.265 | 1.00 | 51.01 | 6 | C |
| ATOM | 1819 | CG | ARG | B | 231 | 23.179 | 27.726 | −11.171 | 1.00 | 57.81 | 6 | C |
| ATOM | 1820 | CD | ARG | B | 231 | 22.506 | 28.843 | −10.382 | 1.00 | 62.05 | 6 | C |
| ATOM | 1821 | NE | ARG | B | 231 | 21.199 | 29.134 | −11.029 | 1.00 | 67.92 | 7 | N |
| ATOM | 1822 | CZ | ARG | B | 231 | 20.244 | 29.845 | −10.398 | 1.00 | 69.53 | 6 | C |
| ATOM | 1823 | NH1 | ARG | B | 231 | 20.384 | 30.350 | −9.147 | 1.00 | 69.58 | 7 | N |
| ATOM | 1824 | NH2 | ARG | B | 231 | 19.124 | 30.023 | −11.107 | 1.00 | 67.94 | 7 | N |
| ATOM | 1825 | N | VAL | B | 232 | 25.214 | 24.956 | −12.600 | 1.00 | 41.93 | 7 | N |
| ATOM | 1826 | CA | VAL | B | 232 | 26.513 | 24.422 | −12.292 | 1.00 | 37.07 | 6 | C |
| ATOM | 1827 | C | VAL | B | 232 | 27.273 | 25.604 | −11.681 | 1.00 | 37.99 | 6 | C |
| ATOM | 1828 | O | VAL | B | 232 | 26.987 | 26.792 | −11.933 | 1.00 | 38.44 | 8 | O |
| ATOM | 1829 | CB | VAL | B | 232 | 27.282 | 23.859 | −13.504 | 1.00 | 37.74 | 6 | C |
| ATOM | 1830 | CG1 | VAL | B | 232 | 28.508 | 23.055 | −13.068 | 1.00 | 37.72 | 6 | C |
| ATOM | 1831 | CG2 | VAL | B | 232 | 26.413 | 22.870 | −14.327 | 1.00 | 33.68 | 6 | C |
| ATOM | 1832 | N | ILE | B | 233 | 28.186 | 25.333 | −10.762 | 1.00 | 31.97 | 7 | N |
| ATOM | 1833 | CA | ILE | B | 233 | 29.030 | 26.225 | −10.086 | 1.00 | 30.98 | 6 | C |
| ATOM | 1834 | C | ILE | B | 233 | 30.469 | 25.614 | −10.087 | 1.00 | 30.13 | 6 | C |
| ATOM | 1835 | O | ILE | B | 233 | 30.547 | 24.562 | −9.455 | 1.00 | 30.79 | 8 | O |
| ATOM | 1836 | CB | ILE | B | 233 | 28.604 | 26.453 | −8.602 | 1.00 | 32.03 | 6 | C |
| ATOM | 1837 | CG1 | ILE | B | 233 | 27.181 | 26.777 | −8.412 | 1.00 | 33.09 | 6 | C |
| ATOM | 1838 | GG2 | ILE | B | 233 | 29.520 | 27.511 | −8.037 | 1.00 | 29.87 | 6 | C |
| ATOM | 1839 | CD1 | ILE | B | 233 | 26.484 | 27.099 | −7.134 | 1.00 | 34.06 | 6 | C |
| ATOM | 1840 | N | GLU | B | 234 | 31.523 | 26.105 | −10.713 | 1.00 | 28.58 | 7 | N |
| ATOM | 1841 | CA | GLU | B | 234 | 32.763 | 25.393 | −10.636 | 1.00 | 30.37 | 6 | C |
| ATOM | 1842 | C | GLU | B | 234 | 33.663 | 25.942 | −9.520 | 1.00 | 29.96 | 6 | C |
| ATOM | 1843 | O | GLU | B | 234 | 33.934 | 27.121 | −9.279 | 1.00 | 32.51 | 8 | O |
| ATOM | 1844 | CB | GLU | B | 234 | 33.593 | 25.433 | −11.956 | 1.00 | 32.68 | 6 | C |
| ATOM | 1845 | CG | GLU | B | 234 | 32.780 | 24.910 | −13.125 | 1.00 | 36.41 | 6 | C |
| ATOM | 1846 | CD | GLU | B | 234 | 33.306 | 25.486 | −14.438 | 1.00 | 39.30 | 6 | C |
| ATOM | 1847 | OE1 | GLU | B | 234 | 34.451 | 25.169 | −14.810 | 1.00 | 40.99 | 8 | O |
| ATOM | 1848 | OE2 | GLU | B | 234 | 32.482 | 26.247 | −15.001 | 1.00 | 43.53 | 8 | O |
| ATOM | 1849 | N | VAL | B | 235 | 34.233 | 25.118 | −8.722 | 1.00 | 25.15 | 7 | N |
| ATOM | 1850 | CA | VAL | B | 235 | 35.182 | 25.458 | −7.721 | 1.00 | 27.49 | 6 | C |
| ATOM | 1851 | C | VAL | B | 235 | 36.527 | 24.748 | −7.963 | 1.00 | 31.55 | 6 | C |
| ATOM | 1852 | O | VAL | B | 235 | 36.597 | 23.523 | −8.137 | 1.00 | 35.85 | 8 | O |
| ATOM | 1853 | CB | VAL | B | 235 | 34.702 | 25.096 | −6.315 | 1.00 | 26.41 | 6 | C |
| ATOM | 1854 | CG1 | VAL | B | 235 | 35.806 | 25.465 | −5.258 | 1.00 | 26.81 | 6 | C |
| ATOM | 1855 | CG2 | VAL | B | 235 | 33.445 | 25.897 | −6.028 | 1.00 | 20.80 | 6 | C |
| ATOM | 1856 | N | ASP | B | 236 | 37.596 | 25.487 | −8.030 | 1.00 | 31.38 | 7 | N |
| ATOM | 1857 | CA | ASP | B | 236 | 38.884 | 24.874 | −8.183 | 1.00 | 31.79 | 6 | C |
| ATOM | 1858 | C | ASP | B | 236 | 39.175 | 24.212 | −6.838 | 1.00 | 32.95 | 6 | C |
| ATOM | 1859 | O | ASP | B | 236 | 39.246 | 24.792 | −5.718 | 1.00 | 31.64 | 8 | O |
| ATOM | 1860 | CB | ASP | B | 236 | 39.891 | 25.896 | −8.649 | 1.00 | 31.49 | 6 | C |
| ATOM | 1861 | CG | ASP | B | 236 | 41.262 | 25.293 | −8.837 | 1.00 | 35.92 | 6 | C |
| ATOM | 1862 | OD1 | ASP | B | 236 | 41.415 | 24.071 | −8.662 | 1.00 | 35.41 | 8 | O |
| ATOM | 1863 | OD2 | ASP | B | 236 | 42.218 | 26.087 | −9.169 | 1.00 | 41.11 | 8 | O |
| ATOM | 1864 | N | THR | B | 237 | 39.433 | 22.892 | −6.906 | 1.00 | 30.69 | 7 | N |
| ATOM | 1865 | CA | THR | B | 237 | 39.770 | 22.098 | −5.783 | 1.00 | 31.00 | 6 | C |
| ATOM | 1866 | C | THR | B | 237 | 41.093 | 21.330 | −5.967 | 1.00 | 33.30 | 6 | C |
| ATOM | 1867 | O | THR | B | 237 | 41.340 | 20.284 | −5.290 | 1.00 | 33.11 | 8 | O |
| ATOM | 1868 | CB | THR | B | 237 | 38.691 | 21.056 | −5.431 | 1.00 | 32.05 | 6 | C |
| ATOM | 1869 | OG1 | THR | B | 237 | 38.400 | 20.105 | −6.480 | 1.00 | 33.86 | 8 | O |
| ATOM | 1870 | CG2 | THR | B | 237 | 37.433 | 21.712 | −4.986 | 1.00 | 28.50 | 6 | C |
| ATOM | 1871 | N | SER | B | 238 | 41.940 | 21.809 | −6.853 | 1.00 | 33.02 | 7 | N |
| ATOM | 1872 | CA | SER | B | 238 | 43.209 | 21.215 | −7.222 | 1.00 | 28.20 | 6 | C |
| ATOM | 1873 | C | SER | B | 238 | 43.978 | 20.887 | −6.002 | 1.00 | 30.04 | 6 | C |
| ATOM | 1874 | O | SER | B | 238 | 44.472 | 19.794 | −5.984 | 1.00 | 35.26 | 8 | O |
| ATOM | 1875 | CB | SER | B | 238 | 44.037 | 22.164 | −8.073 | 1.00 | 27.71 | 6 | C |
| ATOM | 1876 | OG | SER | B | 238 | 44.576 | 23.172 | −7.247 | 1.00 | 30.17 | 8 | O |
| ATOM | 1877 | N | GLU | B | 239 | 44.059 | 21.642 | −4.920 | 1.00 | 32.03 | 7 | N |
| ATOM | 1878 | CA | GLU | B | 239 | 44.873 | 21.168 | −3.772 | 1.00 | 30.79 | 6 | C |
| ATOM | 1879 | C | GLU | B | 239 | 44.329 | 19.978 | −3.077 | 1.00 | 31.99 | 6 | C |
| ATOM | 1880 | O | GLU | B | 239 | 44.978 | 19.200 | −2.421 | 1.00 | 35.69 | 8 | O |
| ATOM | 1881 | CB | GLU | B | 239 | 45.065 | 22.341 | −2.797 | 1.00 | 28.84 | 6 | C |
| ATOM | 1882 | CG | GLU | B | 239 | 45.883 | 23.462 | −3.378 | 1.00 | 29.10 | 6 | C |
| ATOM | 1883 | CD | GLU | B | 239 | 47.013 | 23.103 | −4.317 | 1.00 | 34.18 | 6 | C |
| ATOM | 1884 | OE1 | GLU | B | 239 | 48.130 | 22.773 | −3.858 | 1.00 | 37.03 | 8 | O |
| ATOM | 1885 | OE2 | GLU | B | 239 | 46.744 | 23.168 | −5.545 | 1.00 | 35.24 | 8 | O |
| ATOM | 1886 | N | TYR | B | 240 | 43.043 | 19.709 | −3.051 | 1.00 | 33.88 | 7 | N |
| ATOM | 1887 | CA | TYR | B | 240 | 42.336 | 18.628 | −2.424 | 1.00 | 31.12 | 6 | C |
| ATOM | 1888 | C | TYR | B | 240 | 42.509 | 17.402 | −3.345 | 1.00 | 33.18 | 6 | C |
| ATOM | 1889 | O | TYR | B | 240 | 42.759 | 16.314 | −2.808 | 1.00 | 30.04 | 8 | O |
| ATOM | 1890 | CB | TYR | B | 240 | 40.895 | 19.004 | −2.037 | 1.00 | 27.65 | 6 | C |
| ATOM | 1891 | CG | TYR | B | 240 | 40.914 | 20.188 | −1.068 | 1.00 | 24.69 | 6 | C |
| ATOM | 1892 | CD1 | TYR | B | 240 | 40.740 | 21.496 | −1.477 | 1.00 | 25.31 | 6 | C |
| ATOM | 1893 | CD2 | TYR | B | 240 | 41.117 | 19.988 | 0.290 | 1.00 | 21.30 | 6 | C |
| ATOM | 1894 | CE1 | TYR | B | 240 | 40.776 | 22.534 | −0.523 | 1.00 | 23.85 | 6 | C |
| ATOM | 1895 | CE2 | TYR | B | 240 | 41.156 | 20.962 | 1.225 | 1.00 | 16.53 | 6 | C |
| ATOM | 1896 | CZ | TYR | B | 240 | 40.982 | 22.244 | 0.791 | 1.00 | 21.38 | 6 | C |
| ATOM | 1897 | OH | TYR | B | 240 | 41.006 | 23.285 | 1.676 | 1.00 | 25.33 | 8 | O |
| ATOM | 1898 | N | ARG | B | 241 | 42.535 | 17.576 | −4.665 | 1.00 | 31.36 | 7 | N |
| ATOM | 1899 | CA | ARG | B | 241 | 42.753 | 16.397 | −5.549 | 1.00 | 33.51 | 6 | C |
| ATOM | 1900 | C | ARG | B | 241 | 44.030 | 15.694 | −5.174 | 1.00 | 35.42 | 6 | C |
| ATOM | 1901 | O | ARG | B | 241 | 44.181 | 14.510 | −4.805 | 1.00 | 33.84 | 8 | O |
| ATOM | 1902 | CB | ARG | B | 241 | 42.674 | 16.901 | −6.992 | 1.00 | 35.31 | 6 | C |
| ATOM | 1903 | CG | ARG | B | 241 | 43.130 | 15.792 | −7.969 | 1.00 | 38.56 | 6 | C |
| ATOM | 1904 | CD | ARG | B | 241 | 42.591 | 16.304 | −9.331 | 1.00 | 40.35 | 6 | C |
| ATOM | 1905 | NE | ARG | B | 241 | 43.416 | 15.631 | −10.232 | 1.00 | 46.19 | 7 | N |
| ATOM | 1906 | CZ | ARG | B | 241 | 44.541 | 15.602 | −10.867 | 1.00 | 49.86 | 6 | C |
| ATOM | 1907 | NH1 | ARG | B | 241 | 45.427 | 16.571 | −10.715 | 1.00 | 50.56 | 7 | N |
| ATOM | 1908 | NH2 | ARG | B | 241 | 44.744 | 14.531 | −11.684 | 1.00 | 52.16 | 7 | N |
| ATOM | 1909 | N | LYS | B | 242 | 45.115 | 16.468 | −4.982 | 1.00 | 39.13 | 7 | N |
| ATOM | 1910 | CA | LYS | B | 242 | 46.438 | 16.050 | −4.582 | 1.00 | 36.72 | 6 | C |
| ATOM | 1911 | C | LYS | B | 242 | 46.359 | 15.057 | −3.448 | 1.00 | 38.02 | 6 | C |
| ATOM | 1912 | O | LYS | B | 242 | 47.242 | 14.204 | −3.298 | 1.00 | 41.22 | 8 | O |
| ATOM | 1913 | CB | LYS | B | 242 | 47.346 | 17.215 | −4.171 | 1.00 | 33.98 | 6 | C |
| ATOM | 1914 | CG | LYS | B | 242 | 47.666 | 18.124 | −5.326 | 1.00 | 30.99 | 6 | C |
| ATOM | 1915 | CD | LYS | B | 242 | 48.338 | 19.408 | −4.888 | 1.00 | 33.78 | 6 | C |
| ATOM | 1916 | CE | LYS | B | 242 | 48.653 | 20.298 | −6.069 | 1.00 | 35.39 | 6 | C |
| ATOM | 1917 | NZ | LYS | B | 242 | 49.541 | 21.437 | −5.544 | 1.00 | 40.42 | 7 | N |
| ATOM | 1918 | N | ILE | B | 243 | 45.372 | 15.160 | −2.580 | 1.00 | 36.52 | 7 | N |
| ATOM | 1919 | CA | ILE | B | 243 | 45.381 | 14.149 | −1.503 | 1.00 | 36.49 | 6 | C |
| ATOM | 1920 | C | ILE | B | 243 | 44.083 | 13.350 | −1.621 | 1.00 | 42.73 | 6 | C |
| ATOM | 1921 | O | ILE | B | 243 | 43.357 | 12.985 | −0.676 | 1.00 | 40.86 | 8 | O |
| ATOM | 1922 | CB | ILE | B | 243 | 45.478 | 14.836 | −0.183 | 1.00 | 34.76 | 6 | C |
| ATOM | 1923 | CG1 | ILE | B | 243 | 44.460 | 15.962 | −0.107 | 1.00 | 31.25 | 6 | C |
| ATOM | 1924 | CG2 | ILE | B | 243 | 46.908 | 15.362 | 0.055 | 1.00 | 37.74 | 6 | C |
| ATOM | 1925 | CD1 | ILE | B | 243 | 44.517 | 16.678 | 1.254 | 1.00 | 23.62 | 6 | C |
| ATOM | 1926 | N | ASP | B | 244 | 43.731 | 13.182 | −2.923 | 1.00 | 43.71 | 7 | N |
| ATOM | 1927 | CA | ASP | B | 244 | 42.565 | 12.449 | −3.307 | 1.00 | 45.58 | 6 | C |
| ATOM | 1928 | C | ASP | B | 244 | 41.267 | 12.824 | −2.632 | 1.00 | 41.72 | 6 | C |
| ATOM | 1929 | O | ASP | B | 244 | 40.376 | 12.064 | −2.167 | 1.00 | 37.85 | 8 | O |
| ATOM | 1930 | CB | ASP | B | 244 | 42.884 | 10.920 | −3.131 | 1.00 | 52.26 | 6 | C |
| ATOM | 1931 | CG | ASP | B | 244 | 43.957 | 10.513 | −4.114 | 1.00 | 57.25 | 6 | C |
| ATOM | 1932 | OD1 | ASP | B | 244 | 44.241 | 11.031 | −5.222 | 1.00 | 59.06 | 8 | O |
| ATOM | 1933 | OD2 | ASP | B | 244 | 44.669 | 9.546 | −3.817 | 1.00 | 61.58 | 8 | O |
| ATOM | 1934 | N | GLY | B | 245 | 41.009 | 14.148 | −2.682 | 1.00 | 39.49 | 7 | N |
| ATOM | 1935 | CA | GLY | B | 245 | 39.704 | 14.552 | −2.090 | 1.00 | 35.37 | 6 | C |
| ATOM | 1936 | C | GLY | B | 245 | 39.012 | 15.416 | −3.114 | 1.00 | 32.59 | 6 | C |
| ATOM | 1937 | O | GLY | B | 245 | 39.581 | 15.902 | −4.065 | 1.00 | 33.94 | 8 | O |
| ATOM | 1938 | N | GLY | B | 246 | 37.786 | 15.708 | −2.896 | 1.00 | 29.93 | 7 | N |
| ATOM | 1939 | CA | GLY | B | 246 | 37.048 | 16.585 | −3.785 | 1.00 | 28.70 | 6 | C |
| ATOM | 1940 | C | GLY | B | 246 | 35.852 | 17.169 | −3.066 | 1.00 | 26.29 | 6 | C |
| ATOM | 1941 | O | GLY | B | 246 | 35.727 | 17.157 | −1.868 | 1.00 | 28.74 | 8 | O |
| ATOM | 1942 | N | VAL | B | 247 | 34.983 | 17.748 | −3.903 | 1.00 | 24.88 | 7 | N |
| ATOM | 1943 | CA | VAL | B | 247 | 33.845 | 18.432 | −3.354 | 1.00 | 24.67 | 6 | C |
| ATOM | 1944 | C | VAL | B | 247 | 32.949 | 17.648 | −2.452 | 1.00 | 25.70 | 6 | C |
| ATOM | 1945 | O | VAL | B | 247 | 32.579 | 18.222 | −1.381 | 1.00 | 27.07 | 8 | O |
| ATOM | 1946 | CB | VAL | B | 247 | 33.008 | 19.182 | −4.387 | 1.00 | 25.42 | 6 | C |
| ATOM | 1947 | CG1 | VAL | B | 247 | 33.898 | 20.319 | −4.929 | 1.00 | 21.03 | 6 | C |
| ATOM | 1948 | CG2 | VAL | B | 247 | 32.410 | 18.212 | −5.382 | 1.00 | 22.00 | 6 | C |
| ATOM | 1949 | N | SER | B | 248 | 32.636 | 16.384 | −2.774 | 1.00 | 26.73 | 7 | N |
| ATOM | 1950 | CA | SER | B | 248 | 31.755 | 15.721 | −1.851 | 1.00 | 28.20 | 6 | C |
| ATOM | 1951 | C | SER | B | 248 | 32.445 | 15.468 | −0.529 | 1.00 | 28.24 | 6 | C |
| ATOM | 1952 | O | SER | B | 248 | 31.758 | 15.424 | 0.472 | 1.00 | 27.86 | 8 | O |
| ATOM | 1953 | CB | SER | B | 248 | 31.222 | 14.441 | −2.416 | 1.00 | 31.64 | 6 | C |
| ATOM | 1954 | OG | SER | B | 248 | 31.847 | 13.358 | −1.719 | 1.00 | 36.92 | 8 | O |
| ATOM | 1955 | N | CYS | B | 249 | 33.753 | 15.294 | −0.505 | 1.00 | 31.30 | 7 | N |
| ATOM | 1956 | CA | CYS | B | 249 | 34.405 | 15.008 | 0.779 | 1.00 | 31.73 | 6 | C |
| ATOM | 1957 | C | CYS | B | 249 | 34.431 | 16.145 | 1.749 | 1.00 | 29.65 | 6 | C |
| ATOM | 1958 | O | CYS | B | 249 | 34.692 | 15.990 | 2.961 | 1.00 | 33.43 | 8 | O |
| ATOM | 1959 | CB | CYS | B | 249 | 35.875 | 14.751 | 0.380 | 1.00 | 34.09 | 6 | C |
| ATOM | 1960 | SG | CYS | B | 249 | 36.021 | 13.393 | −0.854 | 1.00 | 42.17 | 16 | S |
| ATOM | 1961 | N | MET | B | 250 | 34.291 | 17.368 | 1.319 | 1.00 | 26.99 | 7 | N |
| ATOM | 1962 | CA | MET | B | 250 | 34.442 | 18.560 | 2.055 | 1.00 | 24.35 | 6 | C |
| ATOM | 1963 | C | MET | B | 250 | 33.228 | 19.031 | 2.795 | 1.00 | 25.38 | 6 | C |
| ATOM | 1964 | O | MET | B | 250 | 33.362 | 20.066 | 3.513 | 1.00 | 29.84 | 8 | O |
| ATOM | 1965 | CB | MET | B | 250 | 35.133 | 19.577 | 1.238 | 1.00 | 27.95 | 6 | C |
| ATOM | 1966 | CG | MET | B | 250 | 36.608 | 19.405 | 0.829 | 1.00 | 30.01 | 6 | C |
| ATOM | 1967 | SD | MET | B | 250 | 36.703 | 20.280 | −0.822 | 1.00 | 34.62 | 16 | S |
| ATOM | 1968 | CE | MET | B | 250 | 37.358 | 21.661 | 0.056 | 1.00 | 28.78 | 6 | C |
| ATOM | 1969 | N | SER | B | 251 | 32.115 | 18.299 | 2.787 | 1.00 | 20.49 | 7 | N |
| ATOM | 1970 | CA | SER | B | 251 | 31.024 | 18.793 | 3.585 | 1.00 | 19.65 | 6 | C |
| ATOM | 1971 | C | SER | B | 251 | 30.111 | 17.651 | 3.933 | 1.00 | 23.84 | 6 | C |
| ATOM | 1972 | O | SER | B | 251 | 30.282 | 16.647 | 3.303 | 1.00 | 23.98 | 8 | O |
| ATOM | 1973 | CB | SER | B | 251 | 30.178 | 19.799 | 2.931 | 1.00 | 20.37 | 6 | C |
| ATOM | 1974 | OG | SER | B | 251 | 29.583 | 19.515 | 1.691 | 1.00 | 21.98 | 8 | O |
| ATOM | 1975 | N | LEU | B | 252 | 29.198 | 17.884 | 4.855 | 1.00 | 26.82 | 7 | N |
| ATOM | 1976 | CA | LEU | B | 252 | 28.146 | 16.886 | 5.211 | 1.00 | 24.00 | 6 | C |
| ATOM | 1977 | C | LEU | B | 252 | 26.922 | 17.718 | 4.852 | 1.00 | 25.46 | 6 | C |
| ATOM | 1978 | O | LEU | B | 252 | 26.790 | 18.935 | 5.077 | 1.00 | 29.33 | 8 | O |
| ATOM | 1979 | CB | LEU | B | 252 | 28.229 | 16.679 | 6.675 | 1.00 | 22.51 | 6 | C |
| ATOM | 1980 | CG | LEU | B | 252 | 28.766 | 15.589 | 7.543 | 1.00 | 25.06 | 6 | C |
| ATOM | 1981 | CD1 | LEU | B | 252 | 29.266 | 14.325 | 6.849 | 1.00 | 19.78 | 6 | C |
| ATOM | 1982 | CD2 | LEU | B | 252 | 29.656 | 16.067 | 8.712 | 1.00 | 21.09 | 6 | C |
| ATOM | 1983 | N | ARG | B | 253 | 25.910 | 17.258 | 4.190 | 1.00 | 26.65 | 7 | N |
| ATOM | 1984 | CA | ARG | B | 253 | 24.670 | 17.784 | 3.703 | 1.00 | 24.05 | 6 | C |
| ATOM | 1985 | C | ARG | B | 253 | 23.480 | 17.011 | 4.215 | 1.00 | 25.30 | 6 | C |
| ATOM | 1986 | O | ARG | B | 253 | 23.508 | 15.792 | 4.067 | 1.00 | 28.18 | 8 | O |
| ATOM | 1987 | CB | ARG | B | 253 | 24.685 | 17.633 | 2.175 | 1.00 | 22.22 | 6 | C |
| ATOM | 1988 | CG | ARG | B | 253 | 25.952 | 18.238 | 1.582 | 1.00 | 26.92 | 6 | C |
| ATOM | 1989 | CD | ARG | B | 253 | 26.350 | 17.944 | 0.182 | 1.00 | 26.27 | 6 | C |
| ATOM | 1990 | NE | ARG | B | 253 | 26.797 | 16.545 | −0.067 | 1.00 | 27.87 | 7 | N |
| ATOM | 1991 | CZ | ARG | B | 253 | 28.084 | 16.225 | 0.185 | 1.00 | 27.75 | 6 | C |
| ATOM | 1992 | NH1 | ARG | B | 253 | 28.466 | 14.977 | −0.043 | 1.00 | 28.89 | 7 | N |
| ATOM | 1993 | NE2 | ARG | B | 253 | 29.087 | 16.967 | 0.644 | 1.00 | 24.41 | 7 | N |
| ATOM | 1994 | N | PHE | B | 254 | 22.423 | 17.610 | 4.775 | 1.00 | 26.50 | 7 | N |
| ATOM | 1995 | CA | PHE | B | 254 | 21.301 | 16.898 | 5.297 | 1.00 | 24.53 | 6 | C |
| ATOM | 1996 | C | PHE | B | 254 | 19.960 | 17.623 | 5.559 | 1.00 | 26.44 | 6 | C |
| ATOM | 1997 | O | PHE | B | 254 | 20.143 | 18.852 | 5.323 | 1.00 | 30.06 | 8 | O |
| ATOM | 1998 | CB | PHE | B | 254 | 21.671 | 16.373 | 6.642 | 1.00 | 22.92 | 6 | C |
| ATOM | 1999 | CG | PHE | B | 254 | 22.341 | 17.144 | 7.699 | 1.00 | 23.97 | 6 | C |
| ATOM | 2000 | CD1 | PHE | B | 254 | 21.590 | 17.732 | 8.708 | 1.00 | 25.52 | 6 | C |
| ATOM | 2001 | CD2 | PHE | B | 254 | 23.686 | 17.288 | 7.728 | 1.00 | 25.50 | 6 | C |
| ATOM | 2002 | CE1 | PHE | B | 254 | 22.168 | 18.449 | 9.714 | 1.00 | 27.04 | 6 | C |
| ATOM | 2003 | CE2 | PHE | B | 254 | 24.264 | 18.025 | 8.757 | 1.00 | 30.49 | 6 | C |
| ATOM | 2004 | CZ | PHE | B | 254 | 23.520 | 18.602 | 9.742 | 1.00 | 28.56 | 6 | C |
| TER | | | | | | | | | | | | |
| ATOM | 1 | O1 | HOH | X | 1 | 24.542 | 23.882 | 9.504 | 1.00 | 35.03 | 8 | O |
| ATOM | 2 | O1 | HOH | X | 2 | 35.318 | 21.073 | 4.942 | 1.00 | 26.50 | 8 | O |
| ATOM | 3 | O1 | HOH | X | 3 | 41.377 | 20.696 | 10.101 | 1.00 | 38.80 | 8 | O |
| ATOM | 4 | O1 | HOH | X | 4 | 42.357 | 19.447 | 7.581 | 1.00 | 33.98 | 8 | O |
| ATOM | 5 | O1 | HOH | X | 5 | 21.470 | 30.853 | −0.085 | 1.00 | 25.96 | 8 | O |
| ATOM | 6 | O1 | HOH | X | 6 | 18.950 | 28.014 | 8.318 | 1.00 | 41.75 | 8 | O |
| ATOM | 7 | O1 | HOH | X | 7 | 43.559 | 13.715 | 9.783 | 1.00 | 26.39 | 8 | O |
| ATOM | 8 | O1 | HOH | X | 8 | 36.771 | 15.500 | 9.672 | 1.00 | 28.13 | 8 | O |
| ATOM | 9 | O1 | HOH | X | 9 | 26.132 | 12.315 | 5.414 | 1.00 | 32.09 | 8 | O |
| ATOM | 10 | O1 | HOH | X | 10 | 21.623 | 13.736 | 4.006 | 1.00 | 29.02 | 8 | O |
| ATOM | 11 | O1 | HOH | X | 11 | 45.271 | 11.728 | 8.694 | 1.00 | 67.76 | 8 | O |
| ATOM | 12 | O1 | HOH | X | 12 | 42.735 | 9.217 | 15.128 | 1.00 | 65.03 | 8 | O |
| ATOM | 13 | O1 | HOH | X | 13 | 32.037 | 19.264 | 16.726 | 1.00 | 40.63 | 8 | O |
| ATOM | 14 | O1 | HOH | X | 14 | 32.892 | 16.947 | 19.263 | 1.00 | 46.29 | 8 | O |
| ATOM | 15 | O1 | HOH | X | 15 | 24.890 | 3.070 | 17.310 | 1.00 | 40.23 | 8 | O |
| ATOM | 16 | O1 | HOH | X | 16 | 32.380 | 5.267 | 15.469 | 1.00 | 49.07 | 8 | O |
| ATOM | 17 | O1 | HOH | X | 17 | 34.753 | 0.707 | 18.627 | 1.00 | 59.61 | 8 | O |
| ATOM | 18 | O1 | HOH | X | 18 | 28.311 | 10.090 | 2.671 | 1.00 | 28.37 | 8 | O |
| ATOM | 19 | O1 | HOH | X | 19 | 25.263 | 10.597 | −0.686 | 1.00 | 41.06 | 8 | O |
| ATOM | 20 | O1 | HOH | X | 20 | 23.460 | 11.655 | −2.756 | 1.00 | 33.02 | 8 | O |
| ATOM | 21 | O1 | HOH | X | 21 | 19.479 | 11.112 | −2.466 | 1.00 | 45.12 | 8 | O |
| ATOM | 22 | O1 | HOH | X | 22 | 22.257 | 13.231 | −4.357 | 1.00 | 44.71 | 8 | O |
| ATOM | 23 | O1 | HOH | X | 23 | 17.776 | 11.619 | −0.278 | 1.00 | 63.16 | 8 | O |
| ATOM | 24 | O1 | HOH | X | 24 | 16.385 | 11.422 | −3.683 | 1.00 | 50.40 | 8 | O |
| ATOM | 25 | O1 | HOH | X | 25 | 39.627 | −1.157 | 7.618 | 1.00 | 41.09 | 8 | O |
| ATOM | 26 | O1 | HOH | X | 26 | 19.010 | −3.375 | 3.753 | 1.00 | 61.26 | 8 | O |
| ATOM | 27 | O1 | HOH | X | 27 | 36.210 | 18.243 | −6.356 | 1.00 | 45.91 | 8 | O |
| ATOM | 28 | O1 | HOH | X | 28 | 35.748 | 16.042 | −7.633 | 1.00 | 44.29 | 8 | O |
| ATOM | 29 | O1 | HOH | X | 29 | 29.319 | 12.535 | 3.046 | 1.00 | 63.89 | 8 | O |
| ATOM | 30 | O1 | HOH | X | 30 | 26.521 | 14.325 | 3.238 | 1.00 | 29.90 | 8 | O |
| ATOM | 31 | O1 | HOH | X | 31 | 28.848 | 14.581 | 2.929 | 1.00 | 58.43 | 8 | O |
| ATOM | 32 | O1 | HOH | X | 32 | 24.945 | 14.476 | −0.833 | 1.00 | 33.94 | 8 | O |
| ATOM | 33 | O1 | HOH | X | 33 | 33.088 | 30.207 | −5.151 | 1.00 | 36.10 | 8 | O |

TABLE III
Structural coordinates for C249S + citrulline

| | | | | | | |
|---|---|---|---|---|---|---|
| CRYST1 | 69.814 | 61.798 | 54.618 | 90.00 | 95.15 | 90.00 |

| | | | | |
|---|---|---|---|---|
| SCALE1 | 0.014324 | 0.000000 | 0.001291 | 0.00000 |
| SCALE2 | 0.000000 | 0.016182 | 0.000000 | 0.00000 |
| SCALE3 | 0.000000 | 0.000000 | 0.018383 | 0.00000 |

| | | | | | | | | | | | | |
|---|---|---|---|---|---|---|---|---|---|---|---|---|
| ATOM | 1 | N | PHE | A | 0 | 10.486 | −6.599 | 29.319 | 1.00 | 34.84 | 7 | N |
| ATOM | 2 | CA | PHE | A | 0 | 11.878 | −6.928 | 28.903 | 1.00 | 33.46 | 6 | C |
| ATOM | 3 | C | PHE | A | 0 | 12.747 | −5.694 | 29.139 | 1.00 | 31.95 | 6 | C |
| ATOM | 4 | O | PHE | A | 0 | 12.228 | −4.596 | 29.328 | 1.00 | 31.99 | 8 | O |
| ATOM | 5 | CB | PHE | A | 0 | 12.002 | −7.409 | 27.466 | 1.00 | 34.68 | 6 | C |
| ATOM | 6 | CG | PHE | A | 0 | 12.507 | −6.481 | 26.401 | 1.00 | 35.21 | 6 | C |
| ATOM | 7 | CD1 | PHE | A | 0 | 13.646 | −6.803 | 25.668 | 1.00 | 35.82 | 6 | C |
| ATOM | 8 | CD2 | PHE | A | 0 | 11.729 | −5.431 | 25.926 | 1.00 | 35.33 | 6 | C |
| ATOM | 9 | CE1 | PHE | A | 0 | 14.025 | −6.038 | 24.578 | 1.00 | 35.25 | 6 | C |
| ATOM | 10 | CE2 | PHE | A | 0 | 11.915 | −4.930 | 24.649 | 1.00 | 35.33 | 6 | C |
| ATOM | 11 | CZ | PHE | A | 0 | 13.122 | −5.158 | 24.018 | 1.00 | 35.03 | 6 | C |
| ATOM | 12 | N | MET | A | 1 | 14.054 | −5.896 | 29.152 | 1.00 | 29.75 | 7 | N |
| ATOM | 13 | CA | MET | A | 1 | 14.993 | −4.809 | 29.339 | 1.00 | 28.18 | 6 | C |
| ATOM | 14 | C | MET | A | 1 | 16.334 | −5.234 | 28.713 | 1.00 | 25.83 | 6 | C |
| ATOM | 15 | O | MET | A | 1 | 16.742 | −6.388 | 28.794 | 1.00 | 25.39 | 8 | O |
| ATOM | 16 | CB | MET | A | 1 | 15.230 | −4.411 | 30.791 | 1.00 | 30.34 | 6 | C |
| ATOM | 17 | CG | MET | A | 1 | 14.044 | −3.804 | 31.523 | 1.00 | 32.69 | 6 | C |
| ATOM | 18 | SD | MET | A | 1 | 13.362 | −2.313 | 30.759 | 1.00 | 35.54 | 16 | S |
| ATOM | 19 | CE | MET | A | 1 | 11.767 | −2.225 | 31.562 | 1.00 | 35.20 | 6 | C |
| ATOM | 20 | N | PHE | A | 2 | 16.993 | −4.265 | 28.110 | 1.00 | 22.15 | 7 | N |
| ATOM | 21 | CA | PHE | A | 2 | 18.304 | −4.520 | 27.516 | 1.00 | 19.52 | 6 | C |
| ATOM | 22 | C | PHE | A | 2 | 19.340 | −4.522 | 28.643 | 1.00 | 18.72 | 6 | C |
| ATOM | 23 | O | PHE | A | 2 | 19.138 | −3.798 | 29.620 | 1.00 | 19.53 | 8 | O |
| ATOM | 24 | CB | PHE | A | 2 | 18.662 | −3.423 | 26.534 | 1.00 | 19.10 | 6 | C |
| ATOM | 25 | CG | PHE | A | 2 | 17.908 | −3.428 | 25.238 | 1.00 | 18.51 | 6 | C |
| ATOM | 26 | CD1 | PHE | A | 2 | 17.469 | −2.233 | 24.684 | 1.00 | 17.79 | 6 | C |
| ATOM | 27 | CD2 | PHE | A | 2 | 17.659 | −4.622 | 24.577 | 1.00 | 17.34 | 6 | C |
| ATOM | 28 | CE1 | PHE | A | 2 | 16.785 | −2.246 | 23.485 | 1.00 | 18.64 | 6 | C |
| ATOM | 29 | CE2 | PHE | A | 2 | 16.969 | −4.631 | 23.381 | 1.00 | 18.28 | 6 | C |
| ATOM | 30 | CZ | PHE | A | 2 | 16.531 | −3.443 | 22.832 | 1.00 | 17.96 | 6 | C |
| ATOM | 31 | N | LYS | A | 3 | 20.396 | −5.315 | 28.503 | 1.00 | 18.95 | 7 | N |
| ATOM | 32 | CA | LYS | A | 3 | 21.424 | −5.342 | 29.536 | 1.00 | 19.96 | 6 | C |
| ATOM | 33 | C | LYS | A | 3 | 22.712 | −4.736 | 28.974 | 1.00 | 19.23 | 6 | C |
| ATOM | 34 | O | LYS | A | 3 | 23.535 | −4.261 | 29.748 | 1.00 | 17.13 | 8 | O |
| ATOM | 35 | CB | LYS | A | 3 | 21.733 | −6.757 | 30.030 | 1.00 | 22.69 | 6 | C |
| ATOM | 36 | CG | LYS | A | 3 | 20.535 | −7.564 | 30.491 | 1.00 | 25.16 | 6 | C |
| ATOM | 37 | CD | LYS | A | 3 | 20.875 | −8.633 | 31.518 | 1.00 | 26.61 | 6 | C |
| ATOM | 38 | CE | LYS | A | 3 | 19.644 | −9.088 | 32.280 | 1.00 | 28.55 | 6 | C |
| ATOM | 39 | NZ | LYS | A | 3 | 18.776 | −10.061 | 31.558 | 1.00 | 28.28 | 7 | N |
| ATOM | 40 | N | HIS | A | 4 | 22.880 | −4.799 | 27.650 | 1.00 | 18.22 | 7 | N |
| ATOM | 41 | CA | HIS | A | 4 | 24.089 | −4.307 | 27.021 | 1.00 | 20.26 | 6 | C |
| ATOM | 42 | C | HIS | A | 4 | 23.921 | −3.535 | 25.713 | 1.00 | 20.77 | 6 | C |
| ATOM | 43 | O | HIS | A | 4 | 22.982 | −3.680 | 24.949 | 1.00 | 21.47 | 8 | O |
| ATOM | 44 | CB | HIS | A | 4 | 24.985 | −5.480 | 26.574 | 1.00 | 21.95 | 6 | C |
| ATOM | 45 | CG | HIS | A | 4 | 25.356 | −6.520 | 27.572 | 1.00 | 22.92 | 6 | C |
| ATOM | 46 | ND1 | HIS | A | 4 | 26.287 | −6.300 | 28.558 | 1.00 | 23.96 | 7 | N |
| ATOM | 47 | CD2 | HIS | A | 4 | 24.930 | −7.793 | 27.743 | 1.00 | 24.02 | 6 | C |
| ATOM | 48 | CE1 | HIS | A | 4 | 26.427 | −7.383 | 29.307 | 1.00 | 24.68 | 6 | C |
| ATOM | 49 | NE2 | HIS | A | 4 | 25.612 | −8.307 | 28.829 | 1.00 | 25.19 | 7 | N |
| ATOM | 50 | N | ILE | A | 5 | 24.950 | −2.742 | 25.449 | 1.00 | 20.42 | 7 | N |
| ATOM | 51 | CA | ILE | A | 5 | 25.109 | −1.940 | 24.259 | 1.00 | 20.43 | 6 | C |
| ATOM | 52 | C | ILE | A | 5 | 26.539 | −2.108 | 23.755 | 1.00 | 19.34 | 6 | C |
| ATOM | 53 | O | ILE | A | 5 | 27.462 | −2.197 | 24.582 | 1.00 | 18.36 | 8 | O |
| ATOM | 54 | CB | ILE | A | 5 | 24.804 | −0.463 | 24.603 | 1.00 | 23.35 | 6 | C |
| ATOM | 55 | CG1 | ILE | A | 5 | 23.297 | −0.280 | 24.336 | 1.00 | 24.22 | 6 | C |
| ATOM | 56 | CG2 | ILE | A | 5 | 25.650 | 0.551 | 23.876 | 1.00 | 24.72 | 6 | C |
| ATOM | 57 | CD1 | ILE | A | 5 | 22.540 | 0.546 | 25.326 | 1.00 | 26.51 | 6 | C |
| ATOM | 58 | N | ILE | A | 6 | 26.721 | −2.158 | 22.444 | 1.00 | 15.55 | 7 | N |
| ATOM | 59 | CA | ILE | A | 6 | 28.027 | −2.202 | 21.813 | 1.00 | 15.75 | 6 | C |
| ATOM | 60 | C | ILE | A | 6 | 28.080 | −0.957 | 20.908 | 1.00 | 15.53 | 6 | C |
| ATOM | 61 | O | ILE | A | 6 | 27.102 | −0.646 | 20.210 | 1.00 | 13.82 | 8 | O |
| ATOM | 62 | CB | ILE | A | 6 | 28.346 | −3.436 | 20.967 | 1.00 | 14.78 | 6 | C |
| ATOM | 63 | CG1 | ILE | A | 6 | 28.335 | −4.695 | 21.838 | 1.00 | 16.97 | 6 | C |
| ATOM | 64 | CG2 | ILE | A | 6 | 29.669 | −3.306 | 20.228 | 1.00 | 16.50 | 6 | C |
| ATOM | 65 | CD1 | ILE | A | 6 | 28.152 | −5.975 | 21.046 | 1.00 | 16.36 | 6 | C |
| ATOM | 66 | N | ALA | A | 7 | 29.204 | −0.263 | 20.884 | 1.00 | 15.16 | 7 | N |
| ATOM | 67 | CA | ALA | A | 7 | 29.380 | 0.929 | 20.045 | 1.00 | 14.15 | 6 | C |
| ATOM | 68 | C | ALA | A | 7 | 30.831 | 1.004 | 19.584 | 1.00 | 15.49 | 6 | C |
| ATOM | 69 | O | ALA | A | 7 | 31.662 | 0.243 | 20.109 | 1.00 | 14.95 | 8 | O |
| ATOM | 70 | CB | ALA | A | 7 | 29.024 | 2.205 | 20.798 | 1.00 | 15.14 | 6 | C |
| ATOM | 71 | N | ARG | A | 8 | 31.172 | 1.887 | 18.648 | 1.00 | 15.36 | 7 | N |
| ATOM | 72 | CA | ARG | A | 8 | 32.589 | 1.971 | 18.231 | 1.00 | 15.29 | 6 | C |
| ATOM | 73 | C | ARG | A | 8 | 32.938 | 3.431 | 17.971 | 1.00 | 16.35 | 6 | C |
| ATOM | 74 | O | ARG | A | 8 | 32.201 | 4.129 | 17.276 | 1.00 | 16.53 | 8 | O |
| ATOM | 75 | CB | ARG | A | 8 | 32.878 | 1.137 | 16.995 | 1.00 | 14.87 | 6 | C |
| ATOM | 76 | CG | ARG | A | 8 | 34.325 | 1.148 | 16.495 | 1.00 | 15.82 | 6 | C |
| ATOM | 77 | CD | ARG | A | 8 | 34.500 | 0.495 | 15.143 | 1.00 | 16.29 | 6 | C |
| ATOM | 78 | NE | ARG | A | 8 | 34.246 | −0.934 | 15.102 | 1.00 | 17.37 | 7 | N |
| ATOM | 79 | CZ | ARG | A | 8 | 35.062 | −1.874 | 15.555 | 1.00 | 18.45 | 6 | C |
| ATOM | 80 | NH1 | ARG | A | 8 | 36.222 | −1.535 | 16.106 | 1.00 | 18.63 | 7 | N |
| ATOM | 81 | NH2 | ARG | A | 8 | 34.711 | −3.155 | 15.457 | 1.00 | 19.40 | 7 | N |
| ATOM | 82 | N | THR | A | 9 | 34.038 | 3.887 | 18.528 | 1.00 | 15.44 | 7 | N |
| ATOM | 83 | CA | THR | A | 9 | 34.504 | 5.259 | 18.332 | 1.00 | 17.16 | 6 | C |
| ATOM | 84 | C | THR | A | 9 | 34.633 | 5.552 | 16.853 | 1.00 | 16.48 | 6 | C |
| ATOM | 85 | O | THR | A | 9 | 35.123 | 4.709 | 16.112 | 1.00 | 16.25 | 8 | O |
| ATOM | 86 | CB | THR | A | 9 | 35.902 | 5.362 | 19.006 | 1.00 | 17.76 | 6 | C |
| ATOM | 87 | OG1 | THR | A | 9 | 35.737 | 4.970 | 20.380 | 1.00 | 16.44 | 8 | O |
| ATOM | 88 | CG2 | THR | A | 9 | 36.488 | 6.759 | 18.971 | 1.00 | 17.88 | 6 | C |
| ATOM | 89 | N | PRO | A | 10 | 34.227 | 6.734 | 16.397 | 1.00 | 17.09 | 7 | N |
| ATOM | 90 | CA | PRO | A | 10 | 34.355 | 7.089 | 14.983 | 1.00 | 16.42 | 6 | C |
| ATOM | 91 | C | PRO | A | 10 | 35.811 | 7.261 | 14.583 | 1.00 | 16.74 | 6 | C |
| ATOM | 92 | O | PRO | A | 10 | 36.640 | 7.694 | 15.412 | 1.00 | 17.10 | 8 | O |
| ATOM | 93 | CB | PRO | A | 10 | 33.564 | 8.391 | 14.888 | 1.00 | 16.10 | 6 | C |
| ATOM | 94 | CG | PRO | A | 10 | 33.641 | 8.980 | 16.255 | 1.00 | 16.61 | 6 | C |
| ATOM | 95 | CD | PRO | A | 10 | 33.627 | 7.809 | 17.216 | 1.00 | 15.55 | 6 | C |
| ATOM | 96 | N | ALA | A | 11 | 36.166 | 6.915 | 13.361 | 1.00 | 16.21 | 7 | N |
| ATOM | 97 | CA | ALA | A | 11 | 37.529 | 7.035 | 12.862 | 1.00 | 17.38 | 6 | C |
| ATOM | 98 | C | ALA | A | 11 | 37.710 | 8.399 | 12.216 | 1.00 | 18.68 | 6 | C |
| ATOM | 99 | O | ALA | A | 11 | 36.721 | 9.086 | 11.900 | 1.00 | 18.73 | 8 | O |
| ATOM | 100 | CB | ALA | A | 11 | 37.803 | 5.969 | 11.813 | 1.00 | 17.84 | 6 | C |
| ATOM | 101 | N | ARG | A | 12 | 38.954 | 8.810 | 11.963 | 1.00 | 19.90 | 7 | N |
| ATOM | 102 | CA | ARG | A | 12 | 39.173 | 10.090 | 11.293 | 1.00 | 20.96 | 6 | C |
| ATOM | 103 | C | ARG | A | 12 | 38.612 | 9.981 | 9.877 | 1.00 | 20.75 | 6 | C |
| ATOM | 104 | O | ARG | A | 12 | 38.130 | 10.978 | 9.312 | 1.00 | 21.87 | 8 | O |
| ATOM | 105 | CB | ARG | A | 12 | 40.647 | 10.467 | 11.208 | 1.00 | 23.96 | 6 | C |
| ATOM | 106 | CG | ARG | A | 12 | 41.267 | 10.928 | 12.525 | 1.00 | 26.00 | 6 | C |
| ATOM | 107 | CD | ARG | A | 12 | 42.765 | 11.172 | 12.374 | 1.00 | 28.75 | 6 | C |
| ATOM | 108 | NE | ARG | A | 12 | 43.518 | 9.959 | 12.086 | 1.00 | 31.76 | 7 | N |
| ATOM | 109 | CZ | ARG | A | 12 | 43.906 | 9.081 | 13.011 | 1.00 | 33.15 | 6 | C |
| ATOM | 110 | NH1 | ARG | A | 12 | 43.601 | 9.298 | 14.282 | 1.00 | 33.71 | 7 | N |
| ATOM | 111 | NH2 | ARG | A | 12 | 44.591 | 7.998 | 12.677 | 1.00 | 33.75 | 7 | N |
| ATOM | 112 | N | SER | A | 13 | 38.655 | 8.792 | 9.275 | 1.00 | 19.58 | 7 | N |
| ATOM | 113 | CA | SER | A | 13 | 38.140 | 8.558 | 7.932 | 1.00 | 19.39 | 6 | C |
| ATOM | 114 | C | SER | A | 13 | 36.625 | 8.667 | 7.822 | 1.00 | 17.92 | 6 | C |
| ATOM | 115 | O | SER | A | 13 | 36.091 | 8.563 | 6.704 | 1.00 | 18.45 | 8 | O |
| ATOM | 116 | CB | SER | A | 13 | 38.610 | 7.228 | 7.340 | 1.00 | 19.66 | 6 | C |
| ATOM | 117 | OG | SER | A | 13 | 38.204 | 6.145 | 8.157 | 1.00 | 19.25 | 8 | O |
| ATOM | 118 | N | LEU | A | 14 | 35.916 | 8.914 | 8.904 | 1.00 | 17.74 | 7 | N |
| ATOM | 119 | CA | LEU | A | 14 | 34.485 | 9.133 | 8.978 | 1.00 | 18.03 | 6 | C |
| ATOM | 120 | C | LEU | A | 14 | 34.095 | 10.251 | 7.999 | 1.00 | 18.19 | 6 | C |
| ATOM | 121 | O | LEU | A | 14 | 33.022 | 10.196 | 7.398 | 1.00 | 17.91 | 8 | O |
| ATOM | 122 | CB | LEU | A | 14 | 34.050 | 9.655 | 10.353 | 1.00 | 18.01 | 6 | C |
| ATOM | 123 | CG | LEU | A | 14 | 32.554 | 9.922 | 10.554 | 1.00 | 18.54 | 6 | C |
| ATOM | 124 | CD1 | LEU | A | 14 | 31.839 | 8.615 | 10.885 | 1.00 | 17.99 | 6 | C |
| ATOM | 125 | CD2 | LEU | A | 14 | 32.252 | 10.951 | 11.626 | 1.00 | 18.06 | 6 | C |
| ATOM | 126 | N | VAL | A | 15 | 34.953 | 11.254 | 7.815 | 1.00 | 18.18 | 7 | N |
| ATOM | 127 | CA | VAL | A | 15 | 34.694 | 12.351 | 6.898 | 1.00 | 19.10 | 6 | C |
| ATOM | 128 | C | VAL | A | 15 | 34.430 | 11.850 | 5.481 | 1.00 | 19.56 | 6 | C |
| ATOM | 129 | O | VAL | A | 15 | 33.736 | 12.528 | 4.711 | 1.00 | 18.47 | 8 | O |
| ATOM | 130 | CB | VAL | A | 15 | 35.814 | 13.405 | 6.865 | 1.00 | 19.70 | 6 | C |
| ATOM | 131 | CG1 | VAL | A | 15 | 36.037 | 13.988 | 8.260 | 1.00 | 19.99 | 6 | C |
| ATOM | 132 | CG2 | VAL | A | 15 | 37.094 | 12.838 | 6.289 | 1.00 | 19.14 | 6 | C |
| ATOM | 133 | N | ASP | A | 16 | 34.991 | 10.692 | 5.130 | 1.00 | 18.79 | 7 | N |
| ATOM | 134 | CA | ASP | A | 16 | 34.823 | 10.050 | 3.850 | 1.00 | 20.81 | 6 | C |
| ATOM | 135 | C | ASP | A | 16 | 33.811 | 8.904 | 3.892 | 1.00 | 19.88 | 6 | C |
| ATOM | 136 | O | ASP | A | 16 | 33.881 | 7.986 | 3.053 | 1.00 | 20.23 | 8 | O |
| ATOM | 137 | CB | ASP | A | 16 | 36.181 | 9.482 | 3.376 | 1.00 | 23.30 | 6 | C |
| ATOM | 138 | CG | ASP | A | 16 | 37.237 | 10.553 | 3.213 | 1.00 | 26.45 | 6 | C |
| ATOM | 139 | OD1 | ASP | A | 16 | 36.877 | 11.686 | 2.819 | 1.00 | 28.16 | 8 | O |
| ATOM | 140 | OD2 | ASP | A | 16 | 38.441 | 10.344 | 3.453 | 1.00 | 28.97 | 8 | O |
| ATOM | 141 | N | GLY | A | 17 | 32.882 | 8.914 | 4.826 | 1.00 | 18.94 | 7 | N |
| ATOM | 142 | CA | GLY | A | 17 | 31.871 | 7.875 | 4.939 | 1.00 | 19.40 | 6 | C |
| ATOM | 143 | C | GLY | A | 17 | 30.930 | 7.922 | 3.726 | 1.00 | 19.51 | 6 | C |
| ATOM | 144 | O | GLY | A | 17 | 30.764 | 8.979 | 3.126 | 1.00 | 18.08 | 8 | O |
| ATOM | 145 | N | LEU | A | 18 | 30.342 | 6.778 | 3.409 | 1.00 | 19.11 | 7 | N |
| ATOM | 146 | CA | LEU | A | 18 | 29.416 | 6.717 | 2.279 | 1.00 | 19.21 | 6 | C |
| ATOM | 147 | C | LEU | A | 18 | 28.210 | 7.621 | 2.504 | 1.00 | 19.09 | 6 | C |
| ATOM | 148 | O | LEU | A | 18 | 27.741 | 7.778 | 3.636 | 1.00 | 17.62 | 8 | O |
| ATOM | 149 | CB | LEU | A | 18 | 28.891 | 5.292 | 2.147 | 1.00 | 18.12 | 6 | C |
| ATOM | 150 | CG | LEU | A | 18 | 29.879 | 4.191 | 1.775 | 1.00 | 18.04 | 6 | C |
| ATOM | 151 | CD1 | LEU | A | 18 | 29.157 | 2.861 | 1.947 | 1.00 | 17.06 | 6 | C |
| ATOM | 152 | CD2 | LEU | A | 18 | 30.434 | 4.384 | 0.374 | 1.00 | 18.90 | 6 | C |
| ATOM | 153 | N | THR | A | 19 | 27.684 | 8.215 | 1.423 | 1.00 | 18.60 | 7 | N |
| ATOM | 154 | CA | THR | A | 19 | 26.518 | 9.054 | 1.583 | 1.00 | 17.96 | 6 | C |
| ATOM | 155 | C | THR | A | 19 | 25.807 | 9.340 | 0.250 | 1.00 | 19.50 | 6 | C |
| ATOM | 156 | O | THR | A | 19 | 26.409 | 9.408 | −0.804 | 1.00 | 18.63 | 8 | O |
| ATOM | 157 | CB | THR | A | 19 | 26.761 | 10.421 | 2.252 | 1.00 | 17.80 | 6 | C |
| ATOM | 158 | OG1 | THR | A | 19 | 25.480 | 11.017 | 2.551 | 1.00 | 16.21 | 8 | O |
| ATOM | 159 | CG2 | THR | A | 19 | 27.550 | 11.364 | 1.373 | 1.00 | 17.31 | 6 | C |
| ATOM | 160 | N | SER | A | 20 | 24.508 | 9.505 | 0.397 | 1.00 | 20.48 | 7 | N |
| ATOM | 161 | CA | SER | A | 20 | 23.650 | 9.837 | −0.738 | 1.00 | 22.66 | 6 | C |
| ATOM | 162 | C | SER | A | 20 | 23.045 | 11.218 | −0.466 | 1.00 | 22.62 | 6 | C |
| ATOM | 163 | O | SER | A | 20 | 22.193 | 11.685 | −1.218 | 1.00 | 22.26 | 8 | O |
| ATOM | 164 | CB | SER | A | 20 | 22.525 | 8.818 | −0.899 | 1.00 | 23.45 | 6 | C |
| ATOM | 165 | OG | SER | A | 20 | 21.673 | 8.836 | 0.245 | 1.00 | 25.71 | 8 | O |
| ATOM | 166 | N | SER | A | 21 | 23.485 | 11.867 | 0.604 | 1.00 | 21.39 | 7 | N |
| ATOM | 167 | CA | SER | A | 21 | 22.967 | 13.184 | 0.963 | 1.00 | 20.49 | 6 | C |
| ATOM | 168 | C | SER | A | 21 | 23.877 | 14.299 | 0.496 | 1.00 | 19.70 | 6 | C |
| ATOM | 169 | O | SER | A | 21 | 24.952 | 14.039 | −0.043 | 1.00 | 17.56 | 8 | O |
| ATOM | 170 | CB | SER | A | 21 | 22.710 | 13.263 | 2.473 | 1.00 | 21.50 | 6 | C |
| ATOM | 171 | OG | SER | A | 21 | 21.736 | 12.259 | 2.769 | 1.00 | 21.95 | 8 | O |
| ATOM | 172 | N | HIS | A | 22 | 23.406 | 15.543 | 0.632 | 1.00 | 20.04 | 7 | N |
| ATOM | 173 | CA | HIS | A | 22 | 24.160 | 16.725 | 0.216 | 1.00 | 21.49 | 6 | C |
| ATOM | 174 | C | HIS | A | 22 | 24.086 | 17.749 | 1.349 | 1.00 | 22.45 | 6 | C |
| ATOM | 175 | O | HIS | A | 22 | 23.735 | 18.929 | 1.221 | 1.00 | 21.66 | 8 | O |
| ATOM | 176 | CB | HIS | A | 22 | 23.660 | 17.272 | −1.128 | 1.00 | 22.85 | 6 | C |
| ATOM | 177 | CG | HIS | A | 22 | 23.723 | 16.305 | −2.277 | 1.00 | 22.85 | 6 | C |
| ATOM | 178 | ND1 | HIS | A | 22 | 24.786 | 16.167 | −3.131 | 1.00 | 23.94 | 7 | N |
| ATOM | 179 | CD2 | HIS | A | 22 | 22.794 | 15.400 | −2.691 | 1.00 | 24.13 | 6 | C |
| ATOM | 180 | CE1 | HIS | A | 22 | 24.520 | 15.224 | −4.025 | 1.00 | 23.85 | 6 | C |
| ATOM | 181 | NE2 | HIS | A | 22 | 23.312 | 14.743 | −3.776 | 1.00 | 23.65 | 7 | N |
| ATOM | 182 | N | LEU | A | 23 | 24.438 | 17.268 | 2.553 | 1.00 | 21.92 | 7 | N |
| ATOM | 183 | CA | LEU | A | 23 | 24.409 | 18.088 | 3.754 | 1.00 | 22.19 | 6 | C |
| ATOM | 184 | C | LEU | A | 23 | 25.779 | 18.520 | 4.251 | 1.00 | 22.87 | 6 | C |
| ATOM | 185 | O | LEU | A | 23 | 25.882 | 19.006 | 5.390 | 1.00 | 23.17 | 8 | O |
| ATOM | 186 | CB | LEU | A | 23 | 23.688 | 17.331 | 4.885 | 1.00 | 22.46 | 6 | C |
| ATOM | 187 | CG | LEU | A | 23 | 22.197 | 17.092 | 4.645 | 1.00 | 22.42 | 6 | C |
| ATOM | 188 | CD1 | LEU | A | 23 | 21.671 | 15.942 | 5.495 | 1.00 | 21.58 | 6 | C |
| ATOM | 189 | CD2 | LEU | A | 23 | 21.428 | 18.375 | 4.922 | 1.00 | 21.84 | 6 | C |
| ATOM | 190 | N | GLY | A | 24 | 26.794 | 18.355 | 3.430 | 1.00 | 23.18 | 7 | N |
| ATOM | 191 | CA | GLY | A | 24 | 28.168 | 18.701 | 3.750 | 1.00 | 23.92 | 6 | C |
| ATOM | 192 | C | GLY | A | 24 | 28.910 | 17.464 | 4.264 | 1.00 | 24.21 | 6 | C |
| ATOM | 193 | O | GLY | A | 24 | 28.275 | 16.416 | 4.415 | 1.00 | 22.41 | 8 | O |
| ATOM | 194 | N | LYS | A | 25 | 30.215 | 17.575 | 4.490 | 1.00 | 23.26 | 7 | N |
| ATOM | 195 | CA | LYS | A | 25 | 30.993 | 16.454 | 4.992 | 1.00 | 21.64 | 6 | C |
| ATOM | 196 | C | LYS | A | 25 | 31.150 | 16.573 | 6.506 | 1.00 | 21.27 | 6 | C |
| ATOM | 197 | O | LYS | A | 25 | 31.271 | 17.683 | 7.029 | 1.00 | 21.25 | 8 | O |
| ATOM | 198 | CB | LYS | A | 25 | 32.386 | 16.392 | 4.365 | 1.00 | 21.93 | 6 | C |
| ATOM | 199 | CG | LYS | A | 25 | 33.251 | 17.550 | 4.612 | 0.00 | 20.00 | 6 | C |
| ATOM | 200 | CD | LYS | A | 25 | 34.594 | 17.375 | 3.920 | 0.00 | 20.00 | 6 | C |
| ATOM | 201 | CE | LYS | A | 25 | 35.490 | 18.582 | 4.145 | 0.00 | 20.00 | 6 | C |
| ATOM | 202 | NZ | LYS | A | 25 | 36.810 | 18.426 | 3.472 | 0.00 | 20.00 | 7 | N |
| ATOM | 203 | N | PRO | A | 26 | 31.165 | 15.434 | 7.183 | 1.00 | 19.41 | 7 | N |
| ATOM | 204 | CA | PRO | A | 26 | 31.312 | 15.404 | 8.622 | 1.00 | 19.94 | 6 | C |
| ATOM | 205 | C | PRO | A | 26 | 32.593 | 16.102 | 9.065 | 1.00 | 19.95 | 6 | C |
| ATOM | 206 | O | PRO | A | 26 | 33.622 | 16.028 | 8.404 | 1.00 | 19.52 | 8 | O |
| ATOM | 207 | CB | PRO | A | 26 | 31.392 | 13.921 | 8.976 | 1.00 | 18.91 | 6 | C |
| ATOM | 208 | CG | PRO | A | 26 | 30.803 | 13.204 | 7.807 | 1.00 | 19.03 | 6 | C |
| ATOM | 209 | CD | PRO | A | 26 | 31.034 | 14.076 | 6.608 | 1.00 | 18.61 | 6 | C |
| ATOM | 210 | N | ASP | A | 27 | 32.489 | 16.780 | 10.192 | 1.00 | 20.51 | 7 | N |
| ATOM | 211 | CA | ASP | A | 27 | 33.614 | 17.464 | 10.837 | 1.00 | 21.51 | 6 | C |
| ATOM | 212 | C | ASP | A | 27 | 34.044 | 16.433 | 11.898 | 1.00 | 20.72 | 6 | C |
| ATOM | 213 | O | ASP | A | 27 | 33.264 | 16.147 | 12.817 | 1.00 | 20.76 | 8 | O |
| ATOM | 214 | CB | ASP | A | 27 | 33.151 | 18.767 | 11.416 | 1.00 | 23.24 | 6 | C |
| ATOM | 215 | CG | ASP | A | 27 | 33.776 | 19.389 | 12.626 | 1.00 | 25.63 | 6 | C |
| ATOM | 216 | OD1 | ASP | A | 27 | 34.786 | 18.959 | 13.216 | 1.00 | 23.50 | 8 | O |
| ATOM | 217 | OD2 | ASP | A | 27 | 33.192 | 20.429 | 13.058 | 1.00 | 28.20 | 8 | O |
| ATOM | 218 | N | TYR | A | 28 | 35.213 | 15.839 | 11.733 | 1.00 | 20.04 | 7 | N |
| ATOM | 219 | CA | TYR | A | 28 | 35.658 | 14.817 | 12.680 | 1.00 | 21.45 | 6 | C |
| ATOM | 220 | C | TYR | A | 28 | 35.631 | 15.244 | 14.129 | 1.00 | 20.40 | 6 | C |
| ATOM | 221 | O | TYR | A | 28 | 35.065 | 14.535 | 14.978 | 1.00 | 19.23 | 8 | O |
| ATOM | 222 | CB | TYR | A | 28 | 37.038 | 14.243 | 12.295 | 1.00 | 24.21 | 6 | C |
| ATOM | 223 | CG | TYR | A | 28 | 37.448 | 13.177 | 13.302 | 1.00 | 27.40 | 6 | C |
| ATOM | 224 | CD1 | TYR | A | 28 | 36.682 | 12.027 | 13.431 | 1.00 | 29.10 | 6 | C |
| ATOM | 225 | CD2 | TYR | A | 28 | 38.565 | 13.320 | 14.108 | 1.00 | 29.34 | 6 | C |
| ATOM | 226 | CE1 | TYR | A | 28 | 37.023 | 11.047 | 14.340 | 1.00 | 30.61 | 6 | C |
| ATOM | 227 | CE2 | TYR | A | 28 | 38.913 | 12.333 | 15.019 | 1.00 | 30.99 | 6 | C |
| ATOM | 228 | CZ | TYR | A | 28 | 38.139 | 11.205 | 15.127 | 1.00 | 31.25 | 6 | C |
| ATOM | 229 | OH | TYR | A | 28 | 38.435 | 10.193 | 16.018 | 1.00 | 34.64 | 8 | O |
| ATOM | 230 | N | ALA | A | 29 | 36.191 | 16.396 | 14.505 | 1.00 | 19.93 | 7 | N |
| ATOM | 231 | CA | ALA | A | 29 | 36.201 | 16.838 | 15.897 | 1.00 | 19.71 | 6 | C |
| ATOM | 232 | C | ALA | A | 29 | 34.799 | 16.938 | 16.478 | 1.00 | 19.88 | 6 | C |
| ATOM | 233 | O | ALA | A | 29 | 34.516 | 16.490 | 17.590 | 1.00 | 18.53 | 8 | O |
| ATOM | 234 | CB | ALA | A | 29 | 36.934 | 18.166 | 16.031 | 1.00 | 21.35 | 6 | C |
| ATOM | 235 | N | LYS | A | 30 | 33.883 | 17.535 | 15.713 | 1.00 | 18.73 | 7 | N |
| ATOM | 236 | CA | LYS | A | 30 | 32.498 | 17.661 | 16.151 | 1.00 | 18.36 | 6 | C |
| ATOM | 237 | C | LYS | A | 30 | 31.818 | 16.307 | 16.267 | 1.00 | 17.40 | 6 | C |
| ATOM | 238 | O | LYS | A | 30 | 31.034 | 16.063 | 17.186 | 1.00 | 16.96 | 8 | O |
| ATOM | 239 | CB | LYS | A | 30 | 31.761 | 18.590 | 15.183 | 1.00 | 19.98 | 6 | C |
| ATOM | 240 | CG | LYS | A | 30 | 30.334 | 18.899 | 15.605 | 1.00 | 22.24 | 6 | C |
| ATOM | 241 | CD | LYS | A | 30 | 29.776 | 19.997 | 14.689 | 1.00 | 25.22 | 6 | C |
| ATOM | 242 | CE | LYS | A | 30 | 28.297 | 20.206 | 14.995 | 1.00 | 26.95 | 6 | C |
| ATOM | 243 | NZ | LYS | A | 30 | 27.628 | 20.937 | 13.871 | 1.00 | 30.17 | 7 | N |
| ATOM | 244 | N | ALA | A | 31 | 32.119 | 15.373 | 15.363 | 1.00 | 17.53 | 7 | N |
| ATOM | 245 | CA | ALA | A | 31 | 31.525 | 14.035 | 15.385 | 1.00 | 17.65 | 6 | C |
| ATOM | 246 | C | ALA | A | 31 | 31.963 | 13.286 | 16.639 | 1.00 | 17.96 | 6 | C |
| ATOM | 247 | O | ALA | A | 31 | 31.189 | 12.542 | 17.227 | 1.00 | 18.21 | 8 | O |
| ATOM | 248 | CB | ALA | A | 31 | 31.939 | 13.224 | 14.174 | 1.00 | 17.70 | 6 | C |
| ATOM | 249 | N | LEU | A | 32 | 33.215 | 13.514 | 17.024 | 1.00 | 18.75 | 7 | N |
| ATOM | 250 | CA | LEU | A | 32 | 33.740 | 12.863 | 18.241 | 1.00 | 19.52 | 6 | C |
| ATOM | 251 | C | LEU | A | 32 | 32.943 | 13.341 | 19.436 | 1.00 | 19.04 | 6 | C |
| ATOM | 252 | O | LEU | A | 32 | 32.533 | 12.550 | 20.290 | 1.00 | 17.48 | 8 | O |
| ATOM | 253 | CB | LEU | A | 32 | 35.223 | 13.198 | 18.319 | 1.00 | 21.98 | 6 | C |
| ATOM | 254 | CG | LEU | A | 32 | 36.086 | 12.424 | 19.307 | 1.00 | 23.27 | 6 | C |
| ATOM | 255 | CD1 | LEU | A | 32 | 36.149 | 10.960 | 18.900 | 1.00 | 22.40 | 6 | C |
| ATOM | 256 | CD2 | LEU | A | 32 | 37.479 | 13.054 | 19.357 | 1.00 | 24.71 | 6 | C |
| ATOM | 257 | N | GLU | A | 33 | 32.692 | 14.652 | 19.520 | 1.00 | 19.01 | 7 | N |
| ATOM | 258 | CA | GLU | A | 33 | 31.927 | 15.189 | 20.637 | 1.00 | 20.58 | 6 | C |
| ATOM | 259 | C | GLU | A | 33 | 30.506 | 14.642 | 20.623 | 1.00 | 19.18 | 6 | C |
| ATOM | 260 | O | GLU | A | 33 | 29.937 | 14.371 | 21.671 | 1.00 | 17.50 | 8 | O |
| ATOM | 261 | CB | GLU | A | 33 | 31.899 | 16.720 | 20.645 | 1.00 | 22.94 | 6 | C |
| ATOM | 262 | CG | GLU | A | 33 | 33.254 | 17.382 | 20.782 | 1.00 | 26.56 | 6 | C |
| ATOM | 263 | CD | GLU | A | 33 | 33.862 | 17.278 | 22.168 | 1.00 | 28.61 | 6 | C |
| ATOM | 264 | OE1 | GLU | A | 33 | 33.145 | 17.353 | 23.181 | 1.00 | 30.54 | 8 | O |
| ATOM | 265 | OE2 | GLU | A | 33 | 35.095 | 17.115 | 22.258 | 1.00 | 31.92 | 8 | O |
| ATOM | 266 | N | GLN | A | 34 | 29.890 | 14.490 | 19.436 | 1.00 | 17.83 | 7 | N |
| ATOM | 267 | CA | GLN | A | 34 | 28.523 | 13.974 | 19.427 | 1.00 | 16.80 | 6 | C |
| ATOM | 268 | C | GLN | A | 34 | 28.466 | 12.504 | 19.842 | 1.00 | 15.85 | 6 | C |
| ATOM | 269 | O | GLN | A | 34 | 27.514 | 12.098 | 20.507 | 1.00 | 15.66 | 8 | O |
| ATOM | 270 | CB | GLN | A | 34 | 27.900 | 14.159 | 18.021 | 1.00 | 17.48 | 6 | C |
| ATOM | 271 | CG | GLN | A | 34 | 27.829 | 15.634 | 17.635 | 1.00 | 17.32 | 6 | C |
| ATOM | 272 | CD | GLN | A | 34 | 27.099 | 15.916 | 16.337 | 1.00 | 18.32 | 6 | C |
| ATOM | 273 | OE1 | GLN | A | 34 | 27.015 | 15.081 | 15.434 | 1.00 | 17.17 | 8 | O |
| ATOM | 274 | NE2 | GLN | A | 34 | 26.559 | 17.143 | 16.241 | 1.00 | 18.36 | 7 | N |
| ATOM | 275 | N | HIS | A | 35 | 29.478 | 11.724 | 19.444 | 1.00 | 15.51 | 7 | N |
| ATOM | 276 | CA | HIS | A | 35 | 29.496 | 10.299 | 19.796 | 1.00 | 15.77 | 6 | C |
| ATOM | 277 | C | HIS | A | 35 | 29.683 | 10.162 | 21.313 | 1.00 | 15.75 | 6 | C |
| ATOM | 278 | O | HIS | A | 35 | 29.108 | 9.302 | 21.960 | 1.00 | 15.76 | 8 | O |
| ATOM | 279 | CB | HIS | A | 35 | 30.587 | 9.540 | 19.043 | 1.00 | 14.81 | 6 | C |
| ATOM | 280 | CG | HIS | A | 35 | 30.523 | 8.046 | 19.187 | 1.00 | 15.47 | 6 | C |
| ATOM | 281 | ND1 | HIS | A | 35 | 29.855 | 7.212 | 18.335 | 1.00 | 15.73 | 7 | N |
| ATOM | 282 | CD2 | HIS | A | 35 | 31.062 | 7.228 | 20.131 | 1.00 | 14.24 | 6 | C |
| ATOM | 283 | CE1 | HIS | A | 35 | 29.976 | 5.948 | 18.723 | 1.00 | 13.38 | 6 | C |
| ATOM | 284 | NE2 | HIS | A | 35 | 30.706 | 5.940 | 19.814 | 1.00 | 17.20 | 7 | N |
| ATOM | 285 | N | ASN | A | 36 | 30.490 | 11.048 | 21.876 | 1.00 | 15.94 | 7 | N |
| ATOM | 286 | CA | ASN | A | 36 | 30.732 | 11.059 | 23.319 | 1.00 | 16.61 | 6 | C |
| ATOM | 287 | C | ASN | A | 36 | 29.408 | 11.397 | 23.982 | 1.00 | 15.95 | 6 | C |
| ATOM | 288 | O | ASN | A | 36 | 28.987 | 10.736 | 24.922 | 1.00 | 15.95 | 8 | O |
| ATOM | 289 | CB | ASN | A | 36 | 31.819 | 12.062 | 23.688 | 1.00 | 17.34 | 6 | C |
| ATOM | 290 | CG | ASN | A | 36 | 32.117 | 12.082 | 25.174 | 1.00 | 19.60 | 6 | C |
| ATOM | 291 | OD1 | ASN | A | 36 | 32.469 | 11.051 | 25.779 | 1.00 | 21.25 | 8 | O |
| ATOM | 292 | ND2 | ASN | A | 36 | 31.966 | 13.260 | 25.768 | 1.00 | 18.15 | 7 | N |
| ATOM | 293 | N | ALA | A | 37 | 28.678 | 12.392 | 23.475 | 1.00 | 16.01 | 7 | N |
| ATOM | 294 | CA | ALA | A | 37 | 27.388 | 12.770 | 24.047 | 1.00 | 15.68 | 6 | C |
| ATOM | 295 | C | ALA | A | 37 | 26.415 | 11.603 | 24.014 | 1.00 | 17.39 | 6 | C |
| ATOM | 296 | O | ALA | A | 37 | 25.585 | 11.406 | 24.912 | 1.00 | 16.75 | 8 | O |
| ATOM | 297 | CB | ALA | A | 37 | 26.839 | 14.004 | 23.346 | 1.00 | 17.33 | 6 | C |
| ATOM | 298 | N | TYR | A | 38 | 26.509 | 10.809 | 22.945 | 1.00 | 17.71 | 7 | N |
| ATOM | 299 | CA | TYR | A | 38 | 25.660 | 9.636 | 22.755 | 1.00 | 17.39 | 6 | C |
| ATOM | 300 | C | TYR | A | 38 | 25.996 | 8.594 | 23.820 | 1.00 | 17.49 | 6 | C |
| ATOM | 301 | O | TYR | A | 38 | 25.100 | 8.003 | 24.412 | 1.00 | 16.46 | 8 | O |
| ATOM | 302 | CB | TYR | A | 38 | 25.855 | 9.098 | 21.360 | 1.00 | 17.02 | 6 | C |
| ATOM | 303 | CG | TYR | A | 38 | 25.177 | 7.849 | 20.871 | 1.00 | 16.94 | 6 | C |
| ATOM | 304 | CD1 | TYR | A | 38 | 23.792 | 7.792 | 20.702 | 1.00 | 16.91 | 6 | C |
| ATOM | 305 | CD2 | TYR | A | 38 | 25.914 | 6.709 | 20.565 | 1.00 | 17.02 | 6 | C |
| ATOM | 306 | CE1 | TYR | A | 38 | 23.189 | 6.628 | 20.240 | 1.00 | 16.10 | 6 | C |
| ATOM | 307 | CE2 | TYR | A | 38 | 25.312 | 5.547 | 20.103 | 1.00 | 16.67 | 6 | C |
| ATOM | 308 | CZ | TYR | A | 38 | 23.944 | 5.520 | 19.945 | 1.00 | 16.35 | 6 | C |
| ATOM | 309 | OH | TYR | A | 38 | 23.312 | 4.374 | 19.484 | 1.00 | 17.37 | 8 | O |
| ATOM | 310 | N | ILE | A | 39 | 27.281 | 8.356 | 24.043 | 1.00 | 17.68 | 7 | N |
| ATOM | 311 | CA | ILE | A | 39 | 27.680 | 7.382 | 25.079 | 1.00 | 17.98 | 6 | C |
| ATOM | 312 | C | ILE | A | 39 | 27.241 | 7.822 | 26.463 | 1.00 | 17.81 | 6 | C |
| ATOM | 313 | O | ILE | A | 39 | 26.765 | 7.013 | 27.282 | 1.00 | 19.30 | 8 | O |
| ATOM | 314 | CB | ILE | A | 39 | 29.200 | 7.148 | 25.007 | 1.00 | 17.61 | 6 | C |
| ATOM | 315 | CG1 | ILE | A | 39 | 29.561 | 6.509 | 23.664 | 1.00 | 17.33 | 6 | C |
| ATOM | 316 | CG2 | ILE | A | 39 | 29.700 | 6.266 | 26.154 | 1.00 | 17.15 | 6 | C |
| ATOM | 317 | CD1 | ILE | A | 39 | 28.921 | 5.158 | 23.399 | 1.00 | 18.02 | 6 | C |
| ATOM | 318 | N | ARG | A | 40 | 27.356 | 9.122 | 26.766 | 1.00 | 17.14 | 7 | N |
| ATOM | 319 | CA | ARG | A | 40 | 26.971 | 9.655 | 28.061 | 1.00 | 16.41 | 6 | C |
| ATOM | 320 | C | ARG | A | 40 | 25.482 | 9.505 | 28.296 | 1.00 | 16.56 | 6 | C |
| ATOM | 321 | O | ARG | A | 40 | 25.062 | 9.310 | 29.429 | 1.00 | 16.61 | 8 | O |
| ATOM | 322 | CB | ARG | A | 40 | 27.391 | 11.125 | 28.242 | 1.00 | 17.11 | 6 | C |
| ATOM | 323 | CG | ARG | A | 40 | 28.898 | 11.298 | 28.127 | 1.00 | 17.31 | 6 | C |
| ATOM | 324 | CD | ARG | A | 40 | 29.322 | 12.711 | 28.542 | 1.00 | 21.96 | 6 | C |
| ATOM | 325 | NE | ARG | A | 40 | 29.211 | 12.837 | 30.001 | 1.00 | 23.03 | 7 | N |
| ATOM | 326 | CZ | ARG | A | 40 | 29.590 | 13.930 | 30.664 | 1.00 | 25.55 | 6 | C |
| ATOM | 327 | NH1 | ARG | A | 40 | 30.085 | 14.959 | 29.986 | 1.00 | 25.55 | 7 | N |
| ATOM | 328 | NH2 | ARG | A | 40 | 29.477 | 14.018 | 31.990 | 1.00 | 26.48 | 7 | N |
| ATOM | 329 | N | ALA | A | 41 | 24.683 | 9.621 | 27.232 | 1.00 | 15.54 | 7 | N |
| ATOM | 330 | CA | ALA | A | 41 | 23.239 | 9.421 | 27.333 | 1.00 | 15.30 | 6 | C |
| ATOM | 331 | C | ALA | A | 41 | 23.014 | 7.936 | 27.632 | 1.00 | 15.16 | 6 | C |
| ATOM | 332 | O | ALA | A | 41 | 22.207 | 7.563 | 28.497 | 1.00 | 16.83 | 8 | O |
| ATOM | 333 | CB | ALA | A | 41 | 22.590 | 9.775 | 25.996 | 1.00 | 14.59 | 6 | C |
| ATOM | 334 | N | LEU | A | 42 | 23.717 | 7.048 | 26.927 | 1.00 | 15.85 | 7 | N |
| ATOM | 335 | CA | LEU | A | 42 | 23.587 | 5.607 | 27.119 | 1.00 | 16.10 | 6 | C |
| ATOM | 336 | C | LEU | A | 42 | 24.051 | 5.158 | 28.490 | 1.00 | 16.83 | 6 | C |
| ATOM | 337 | O | LEU | A | 42 | 23.558 | 4.158 | 29.027 | 1.00 | 17.17 | 8 | O |
| ATOM | 338 | CB | LEU | A | 42 | 24.309 | 4.790 | 26.035 | 1.00 | 16.72 | 6 | C |
| ATOM | 339 | CG | LEU | A | 42 | 23.614 | 4.796 | 24.666 | 1.00 | 17.58 | 6 | C |
| ATOM | 340 | CD1 | LEU | A | 42 | 24.590 | 4.456 | 23.552 | 1.00 | 16.82 | 6 | C |
| ATOM | 341 | CD2 | LEU | A | 42 | 22.410 | 3.865 | 24.656 | 1.00 | 16.86 | 6 | C |
| ATOM | 342 | N | GLN | A | 43 | 24.942 | 5.909 | 29.146 | 1.00 | 16.42 | 7 | N |
| ATOM | 343 | CA | GLN | A | 43 | 25.388 | 5.594 | 30.493 | 1.00 | 17.10 | 6 | C |
| ATOM | 344 | C | GLN | A | 43 | 24.286 | 5.802 | 31.533 | 1.00 | 17.80 | 6 | C |
| ATOM | 345 | O | GLN | A | 43 | 24.437 | 5.348 | 32.671 | 1.00 | 17.36 | 8 | O |
| ATOM | 346 | CB | GLN | A | 43 | 26.583 | 6.478 | 30.866 | 1.00 | 17.64 | 6 | C |
| ATOM | 347 | CG | GLN | A | 43 | 27.929 | 6.099 | 30.301 | 1.00 | 18.31 | 6 | C |
| ATOM | 348 | CD | GLN | A | 43 | 29.028 | 7.089 | 30.652 | 1.00 | 19.64 | 6 | C |
| ATOM | 349 | OE1 | GLN | A | 43 | 29.661 | 7.005 | 31.727 | 1.00 | 21.94 | 8 | O |
| ATOM | 350 | NE2 | GLN | A | 43 | 29.300 | 8.029 | 29.787 | 1.00 | 18.72 | 7 | N |
| ATOM | 351 | N | THR | A | 44 | 23.193 | 6.483 | 31.181 | 1.00 | 18.12 | 7 | N |
| ATOM | 352 | CA | THR | A | 44 | 22.071 | 6.723 | 32.068 | 1.00 | 19.00 | 6 | C |
| ATOM | 353 | C | THR | A | 44 | 20.923 | 5.744 | 31.841 | 1.00 | 19.45 | 6 | C |
| ATOM | 354 | O | THR | A | 44 | 19.869 | 5.851 | 32.486 | 1.00 | 20.44 | 8 | O |
| ATOM | 355 | CB | THR | A | 44 | 21.490 | 8.144 | 31.922 | 1.00 | 19.93 | 6 | C |
| ATOM | 356 | OG1 | THR | A | 44 | 20.755 | 8.315 | 30.700 | 1.00 | 20.90 | 8 | O |
| ATOM | 357 | CG2 | THR | A | 44 | 22.609 | 9.181 | 31.919 | 1.00 | 20.24 | 6 | C |
| ATOM | 358 | N | CYH | A | 45 | 21.095 | 4.763 | 30.964 | 1.00 | 19.74 | 7 | N |
| ATOM | 359 | CA | CYH | A | 45 | 20.033 | 3.814 | 30.657 | 1.00 | 20.07 | 6 | C |
| ATOM | 360 | C | CYH | A | 45 | 20.125 | 2.441 | 31.286 | 1.00 | 20.92 | 6 | C |
| ATOM | 361 | O | CYH | A | 45 | 19.515 | 1.491 | 30.801 | 1.00 | 20.53 | 8 | O |
| ATOM | 362 | CB | CYH | A | 45 | 19.914 | 3.676 | 29.127 | 1.00 | 19.22 | 6 | C |
| ATOM | 363 | SG | CYH | A | 45 | 19.561 | 5.230 | 28.274 | 1.00 | 19.20 | 16 | S |
| ATOM | 364 | N | ASP | A | 46 | 20.864 | 2.273 | 32.373 | 1.00 | 21.40 | 7 | N |
| ATOM | 365 | CA | ASP | A | 46 | 20.985 | 1.018 | 33.081 | 1.00 | 22.30 | 6 | C |
| ATOM | 366 | C | ASP | A | 46 | 21.443 | −0.153 | 32.235 | 1.00 | 21.77 | 6 | C |
| ATOM | 367 | O | ASP | A | 46 | 20.938 | −1.269 | 32.315 | 1.00 | 20.87 | 8 | O |
| ATOM | 368 | CB | ASP | A | 46 | 19.659 | 0.716 | 33.809 | 1.00 | 26.21 | 6 | C |
| ATOM | 369 | CG | ASP | A | 46 | 19.417 | 1.826 | 34.832 | 1.00 | 29.62 | 6 | C |
| ATOM | 370 | OD1 | ASP | A | 46 | 20.399 | 2.505 | 35.205 | 1.00 | 32.76 | 8 | O |
| ATOM | 371 | OD2 | ASP | A | 46 | 18.253 | 1.996 | 35.232 | 1.00 | 31.85 | 8 | O |
| ATOM | 372 | N | VAL | A | 47 | 22.457 | 0.083 | 31.404 | 1.00 | 19.76 | 7 | N |
| ATOM | 373 | CA | VAL | A | 47 | 23.029 | −0.962 | 30.571 | 1.00 | 19.18 | 6 | C |
| ATOM | 374 | C | VAL | A | 47 | 24.547 | −0.894 | 30.721 | 1.00 | 18.35 | 6 | C |
| ATOM | 375 | O | VAL | A | 47 | 25.029 | 0.175 | 31.119 | 1.00 | 18.07 | 8 | O |
| ATOM | 376 | CB | VAL | A | 47 | 22.724 | −0.745 | 29.073 | 1.00 | 18.59 | 6 | C |
| ATOM | 377 | CG1 | VAL | A | 47 | 21.245 | −0.960 | 28.763 | 1.00 | 19.18 | 6 | C |
| ATOM | 378 | CG2 | VAL | A | 47 | 23.135 | 0.658 | 28.632 | 1.00 | 18.31 | 6 | C |
| ATOM | 379 | N | ASP | A | 48 | 25.287 | −1.965 | 30.429 | 1.00 | 18.46 | 7 | N |
| ATOM | 380 | CA | ASP | A | 48 | 26.746 | −1.760 | 30.469 | 1.00 | 20.15 | 6 | C |
| ATOM | 381 | C | ASP | A | 48 | 27.075 | −1.528 | 28.990 | 1.00 | 19.10 | 6 | C |
| ATOM | 382 | O | ASP | A | 48 | 26.306 | −1.942 | 28.112 | 1.00 | 17.75 | 8 | O |
| ATOM | 383 | CB | ASP | A | 48 | 27.629 | −2.740 | 31.181 | 1.00 | 23.02 | 6 | C |
| ATOM | 384 | CG | ASP | A | 48 | 27.243 | −4.163 | 30.870 | 1.00 | 25.01 | 6 | C |
| ATOM | 385 | OD1 | ASP | A | 48 | 27.140 | −4.354 | 29.641 | 1.00 | 27.97 | 8 | O |
| ATOM | 386 | OD2 | ASP | A | 48 | 27.079 | −4.968 | 31.807 | 1.00 | 26.87 | 8 | O |
| ATOM | 387 | N | ILE | A | 49 | 28.158 | −0.822 | 28.730 | 1.00 | 17.74 | 7 | N |
| ATOM | 388 | CA | ILE | A | 49 | 28.545 | −0.425 | 27.394 | 1.00 | 17.46 | 6 | C |
| ATOM | 389 | C | ILE | A | 49 | 29.886 | −1.009 | 26.993 | 1.00 | 18.13 | 6 | C |
| ATOM | 390 | O | ILE | A | 49 | 30.792 | −0.980 | 27.830 | 1.00 | 19.06 | 8 | O |
| ATOM | 391 | CB | ILE | A | 49 | 28.656 | 1.127 | 27.403 | 1.00 | 17.25 | 6 | C |
| ATOM | 392 | CG1 | ILE | A | 49 | 27.285 | 1.761 | 27.656 | 1.00 | 16.41 | 6 | C |
| ATOM | 393 | CG2 | ILE | A | 49 | 29.307 | 1.683 | 26.152 | 1.00 | 16.85 | 6 | C |
| ATOM | 394 | CD1 | ILE | A | 49 | 27.277 | 3.115 | 28.344 | 1.00 | 17.77 | 6 | C |
| ATOM | 395 | N | THR | A | 50 | 29.954 | −1.559 | 25.795 | 1.00 | 17.09 | 7 | N |
| ATOM | 396 | CA | THR | A | 50 | 31.230 | −2.089 | 25.290 | 1.00 | 16.16 | 6 | C |
| ATOM | 397 | C | THR | A | 50 | 31.599 | −1.115 | 24.173 | 1.00 | 16.37 | 6 | C |
| ATOM | 398 | O | THR | A | 50 | 30.912 | −1.092 | 23.166 | 1.00 | 14.97 | 8 | O |
| ATOM | 399 | CB | THR | A | 50 | 31.123 | −3.529 | 24.782 | 1.00 | 16.19 | 6 | C |
| ATOM | 400 | OG1 | THR | A | 50 | 30.762 | −4.342 | 25.907 | 1.00 | 16.27 | 8 | O |
| ATOM | 401 | CG2 | THR | A | 50 | 32.432 | −3.990 | 24.158 | 1.00 | 15.83 | 6 | C |
| ATOM | 402 | N | LEU | A | 51 | 32.634 | −0.314 | 24.355 | 1.00 | 16.48 | 7 | N |
| ATOM | 403 | CA | LEU | A | 51 | 33.040 | 0.676 | 23.370 | 1.00 | 16.70 | 6 | C |
| ATOM | 404 | C | LEU | A | 51 | 34.300 | 0.244 | 22.656 | 1.00 | 17.07 | 6 | C |
| ATOM | 405 | O | LEU | A | 51 | 35.358 | 0.205 | 23.276 | 1.00 | 17.10 | 8 | O |
| ATOM | 406 | CB | LEU | A | 51 | 33.205 | 2.001 | 24.126 | 1.00 | 18.72 | 6 | C |
| ATOM | 407 | CG | LEU | A | 51 | 33.676 | 3.241 | 23.377 | 1.00 | 19.48 | 6 | C |
| ATOM | 408 | CD1 | LEU | A | 51 | 32.832 | 3.523 | 22.139 | 1.00 | 19.63 | 6 | C |
| ATOM | 409 | CD2 | LEU | A | 51 | 33.655 | 4.441 | 24.331 | 1.00 | 18.92 | 6 | C |
| ATOM | 410 | N | LEU | A | 52 | 34.165 | −0.096 | 21.370 | 1.00 | 16.03 | 7 | N |
| ATOM | 411 | CA | LEU | A | 52 | 35.329 | −0.548 | 20.590 | 1.00 | 16.58 | 6 | C |
| ATOM | 412 | C | LEU | A | 52 | 36.124 | 0.601 | 20.007 | 1.00 | 17.29 | 6 | C |
| ATOM | 413 | O | LEU | A | 52 | 35.593 | 1.686 | 19.808 | 1.00 | 17.67 | 8 | O |
| ATOM | 414 | CB | LEU | A | 52 | 34.820 | −1.503 | 19.493 | 1.00 | 16.47 | 6 | C |
| ATOM | 415 | CG | LEU | A | 52 | 33.908 | −2.606 | 20.102 | 1.00 | 17.34 | 6 | C |
| ATOM | 416 | CD1 | LEU | A | 52 | 33.290 | −3.428 | 18.988 | 1.00 | 17.64 | 6 | C |
| ATOM | 417 | CD2 | LEU | A | 52 | 34.774 | −3.479 | 21.012 | 1.00 | 17.88 | 6 | C |
| ATOM | 418 | N | PRO | A | 53 | 37.408 | 0.359 | 19.735 | 1.00 | 18.13 | 7 | N |
| ATOM | 419 | CA | PRO | A | 53 | 38.300 | 1.358 | 19.174 | 1.00 | 18.68 | 6 | C |
| ATOM | 420 | C | PRO | A | 53 | 38.012 | 1.662 | 17.708 | 1.00 | 18.23 | 6 | C |
| ATOM | 421 | O | PRO | A | 53 | 37.458 | 0.853 | 16.954 | 1.00 | 18.12 | 8 | O |
| ATOM | 422 | CB | PRO | A | 53 | 39.676 | 0.724 | 19.328 | 1.00 | 19.31 | 6 | C |
| ATOM | 423 | CG | PRO | A | 53 | 39.425 | −0.749 | 19.319 | 1.00 | 20.13 | 6 | C |
| ATOM | 424 | CD | PRO | A | 53 | 38.078 | −0.942 | 19.971 | 1.00 | 19.07 | 6 | C |
| ATOM | 425 | N | PRO | A | 54 | 38.450 | 2.832 | 17.256 | 1.00 | 19.31 | 7 | N |
| ATOM | 426 | CA | PRO | A | 54 | 38.249 | 3.235 | 15.873 | 1.00 | 19.69 | 6 | C |
| ATOM | 427 | C | PRO | A | 54 | 39.051 | 2.383 | 14.917 | 1.00 | 20.19 | 6 | C |
| ATOM | 428 | O | PRO | A | 54 | 40.099 | 1.822 | 15.273 | 1.00 | 19.58 | 8 | O |
| ATOM | 429 | CB | PRO | A | 54 | 38.709 | 4.688 | 15.837 | 1.00 | 19.42 | 6 | C |
| ATOM | 430 | CG | PRO | A | 54 | 39.619 | 4.853 | 17.003 | 1.00 | 19.42 | 6 | C |
| ATOM | 431 | CD | PRO | A | 54 | 39.159 | 3.871 | 18.053 | 1.00 | 19.10 | 6 | C |
| ATOM | 432 | N | ASP | A | 55 | 38.590 | 2.273 | 13.669 | 1.00 | 20.51 | 7 | N |
| ATOM | 433 | CA | ASP | A | 55 | 39.334 | 1.500 | 12.671 | 1.00 | 20.52 | 6 | C |
| ATOM | 434 | C | ASP | A | 55 | 39.378 | 2.393 | 11.434 | 1.00 | 20.75 | 6 | C |
| ATOM | 435 | O | ASP | A | 55 | 38.343 | 2.526 | 10.787 | 1.00 | 19.74 | 8 | O |
| ATOM | 436 | CB | ASP | A | 55 | 38.715 | 0.158 | 12.336 | 1.00 | 21.30 | 6 | C |
| ATOM | 437 | CG | ASP | A | 55 | 39.668 | −0.725 | 11.545 | 1.00 | 22.15 | 6 | C |
| ATOM | 438 | OD1 | ASP | A | 55 | 40.404 | −0.215 | 10.677 | 1.00 | 22.35 | 8 | O |
| ATOM | 439 | OD2 | ASP | A | 55 | 39.677 | −1.947 | 11.808 | 1.00 | 22.23 | 8 | O |
| ATOM | 440 | N | GLU | A | 56 | 40.530 | 2.981 | 11.127 | 1.00 | 20.95 | 7 | N |
| ATOM | 441 | CA | GLU | A | 56 | 40.652 | 3.872 | 9.983 | 1.00 | 21.91 | 6 | C |
| ATOM | 442 | C | GLU | A | 56 | 40.374 | 3.266 | 8.628 | 1.00 | 22.14 | 6 | C |
| ATOM | 443 | O | GLU | A | 56 | 40.005 | 3.976 | 7.675 | 1.00 | 22.30 | 8 | O |
| ATOM | 444 | CB | GLU | A | 56 | 42.073 | 4.494 | 9.986 | 1.00 | 21.51 | 6 | C |
| ATOM | 445 | CG | GLU | A | 56 | 42.320 | 5.381 | 11.200 | 1.00 | 22.25 | 6 | C |
| ATOM | 446 | CD | GLU | A | 56 | 41.351 | 6.548 | 11.256 | 1.00 | 22.92 | 6 | C |
| ATOM | 447 | OE1 | GLU | A | 56 | 40.899 | 6.988 | 10.168 | 1.00 | 23.40 | 8 | O |
| ATOM | 448 | OE2 | GLU | A | 56 | 41.037 | 7.030 | 12.361 | 1.00 | 23.13 | 8 | O |
| ATOM | 449 | N | ARG | A | 57 | 40.512 | 1.952 | 8.488 | 1.00 | 21.32 | 7 | N |
| ATOM | 450 | CA | ARG | A | 57 | 40.253 | 1.275 | 7.232 | 1.00 | 22.00 | 6 | C |
| ATOM | 451 | C | ARG | A | 57 | 38.764 | 1.275 | 6.896 | 1.00 | 20.96 | 6 | C |
| ATOM | 452 | O | ARG | A | 57 | 38.411 | 1.000 | 5.753 | 1.00 | 20.22 | 8 | O |
| ATOM | 453 | CB | ARG | A | 57 | 40.701 | −0.191 | 7.284 | 1.00 | 23.05 | 6 | C |
| ATOM | 454 | CG | ARG | A | 57 | 42.168 | −0.438 | 7.590 | 1.00 | 25.32 | 6 | C |
| ATOM | 455 | CD | ARG | A | 57 | 42.410 | −1.946 | 7.718 | 1.00 | 26.59 | 6 | C |
| ATOM | 456 | NE | ARG | A | 57 | 41.705 | −2.457 | 8.897 | 1.00 | 27.97 | 7 | N |
| ATOM | 457 | CZ | ARG | A | 57 | 41.826 | −3.688 | 9.363 | 1.00 | 29.42 | 6 | C |
| ATOM | 458 | NH1 | ARG | A | 57 | 42.643 | −4.531 | 8.730 | 1.00 | 30.82 | 7 | N |
| ATOM | 459 | NH2 | ARG | A | 57 | 41.147 | −4.058 | 10.439 | 1.00 | 29.69 | 7 | N |
| ATOM | 460 | N | PHE | A | 58 | 37.886 | 1.509 | 7.860 | 1.00 | 19.02 | 7 | N |
| ATOM | 461 | CA | PHE | A | 58 | 36.451 | 1.497 | 7.665 | 1.00 | 18.61 | 6 | C |
| ATOM | 462 | C | PHE | A | 58 | 35.799 | 2.790 | 8.130 | 1.00 | 17.30 | 6 | C |
| ATOM | 463 | O | PHE | A | 58 | 35.326 | 2.928 | 9.254 | 1.00 | 19.92 | 8 | O |
| ATOM | 464 | CB | PHE | A | 58 | 35.825 | 0.291 | 8.398 | 1.00 | 18.10 | 6 | C |
| ATOM | 465 | CG | PHE | A | 58 | 36.324 | −1.029 | 7.867 | 1.00 | 19.72 | 6 | C |
| ATOM | 466 | CD1 | PHE | A | 58 | 37.331 | −1.703 | 8.546 | 1.00 | 19.32 | 6 | C |
| ATOM | 467 | CD2 | PHE | A | 58 | 35.815 | −1.580 | 6.705 | 1.00 | 19.53 | 6 | C |
| ATOM | 468 | CE1 | PHE | A | 58 | 37.818 | −2.905 | 8.082 | 1.00 | 19.70 | 6 | C |
| ATOM | 469 | CE2 | PHE | A | 58 | 36.299 | −2.783 | 6.233 | 1.00 | 19.56 | 6 | C |
| ATOM | 470 | CZ | PHE | A | 58 | 37.297 | −3.441 | 6.921 | 1.00 | 19.55 | 6 | C |
| ATOM | 471 | N | PRO | A | 59 | 35.713 | 3.782 | 7.235 | 1.00 | 17.17 | 7 | N |
| ATOM | 472 | CA | PRO | A | 59 | 35.118 | 5.071 | 7.525 | 1.00 | 16.18 | 6 | C |
| ATOM | 473 | C | PRO | A | 59 | 33.761 | 5.046 | 8.204 | 1.00 | 15.94 | 6 | C |
| ATOM | 474 | O | PRO | A | 59 | 33.440 | 5.870 | 9.079 | 1.00 | 15.97 | 8 | O |
| ATOM | 475 | CB | PRO | A | 59 | 34.949 | 5.669 | 6.114 | 1.00 | 17.24 | 6 | C |
| ATOM | 476 | CG | PRO | A | 59 | 36.128 | 5.133 | 5.372 | 1.00 | 17.95 | 6 | C |
| ATOM | 477 | CD | PRO | A | 59 | 36.256 | 3.696 | 5.844 | 1.00 | 16.89 | 6 | C |
| ATOM | 478 | N | ASP | A | 60 | 32.898 | 4.102 | 7.819 | 1.00 | 15.33 | 7 | N |
| ATOM | 479 | CA | ASP | A | 60 | 31.553 | 3.978 | 8.379 | 1.00 | 15.01 | 6 | C |
| ATOM | 480 | C | ASP | A | 60 | 31.451 | 2.947 | 9.485 | 1.00 | 15.24 | 6 | C |
| ATOM | 481 | O | ASP | A | 60 | 30.338 | 2.601 | 9.916 | 1.00 | 14.28 | 8 | O |
| ATOM | 482 | CB | ASP | A | 60 | 30.598 | 3.567 | 7.240 | 1.00 | 15.95 | 6 | C |
| ATOM | 483 | CG | ASP | A | 60 | 30.338 | 4.672 | 6.229 | 1.00 | 16.82 | 6 | C |
| ATOM | 484 | OD1 | ASP | A | 60 | 29.712 | 5.661 | 6.662 | 1.00 | 17.32 | 8 | O |
| ATOM | 485 | OD2 | ASP | A | 60 | 30.750 | 4.526 | 5.063 | 1.00 | 18.51 | 8 | O |
| ATOM | 486 | N | SER | A | 61 | 32.572 | 2.471 | 10.051 | 1.00 | 16.42 | 7 | N |
| ATOM | 487 | CA | SER | A | 61 | 32.540 | 1.452 | 11.102 | 1.00 | 16.87 | 6 | C |
| ATOM | 488 | C | SER | A | 61 | 31.916 | 1.860 | 12.418 | 1.00 | 16.66 | 6 | C |
| ATOM | 489 | O | SER | A | 61 | 31.558 | 1.028 | 13.274 | 1.00 | 15.45 | 8 | O |
| ATOM | 490 | CB | SER | A | 61 | 33.919 | 0.792 | 11.243 | 1.00 | 16.12 | 6 | C |
| ATOM | 491 | OG | SER | A | 61 | 34.881 | 1.734 | 11.651 | 1.00 | 16.56 | 8 | O |
| ATOM | 492 | N | VAL | A | 62 | 31.646 | 3.141 | 12.640 | 1.00 | 16.03 | 7 | N |
| ATOM | 493 | CA | VAL | A | 62 | 30.966 | 3.691 | 13.791 | 1.00 | 15.34 | 6 | C |
| ATOM | 494 | C | VAL | A | 62 | 29.528 | 3.181 | 13.774 | 1.00 | 14.51 | 6 | C |
| ATOM | 495 | O | VAL | A | 62 | 28.872 | 3.054 | 14.809 | 1.00 | 14.22 | 8 | O |
| ATOM | 496 | CB | VAL | A | 62 | 30.957 | 5.233 | 13.779 | 1.00 | 15.67 | 6 | C |
| ATOM | 497 | CG1 | VAL | A | 62 | 30.222 | 5.782 | 12.552 | 1.00 | 16.45 | 6 | C |
| ATOM | 498 | CG2 | VAL | A | 62 | 30.334 | 5.800 | 15.041 | 1.00 | 15.95 | 6 | C |
| ATOM | 499 | N | PHE | A | 63 | 29.003 | 2.865 | 12.578 | 1.00 | 14.14 | 7 | N |
| ATOM | 500 | CA | PHE | A | 63 | 27.651 | 2.354 | 12.440 | 1.00 | 14.70 | 6 | C |
| ATOM | 501 | C | PHE | A | 63 | 27.618 | 0.845 | 12.632 | 1.00 | 15.04 | 6 | C |
| ATOM | 502 | O | PHE | A | 63 | 27.327 | 0.032 | 11.744 | 1.00 | 14.19 | 8 | O |
| ATOM | 503 | CB | PHE | A | 63 | 27.057 | 2.727 | 11.066 | 1.00 | 14.74 | 6 | C |
| ATOM | 504 | CG | PHE | A | 63 | 27.046 | 4.232 | 10.883 | 1.00 | 14.96 | 6 | C |
| ATOM | 505 | CD1 | PHE | A | 63 | 27.646 | 4.800 | 9.769 | 1.00 | 16.53 | 6 | C |
| ATOM | 506 | CD2 | PHE | A | 63 | 26.441 | 5.039 | 11.823 | 1.00 | 16.14 | 6 | C |
| ATOM | 507 | CE1 | PHE | A | 63 | 27.635 | 6.176 | 9.605 | 1.00 | 16.87 | 6 | C |
| ATOM | 508 | CE2 | PHE | A | 63 | 26.426 | 6.419 | 11.665 | 1.00 | 17.07 | 6 | C |
| ATOM | 509 | CZ | PHE | A | 63 | 27.026 | 6.978 | 10.553 | 1.00 | 16.78 | 6 | C |
| ATOM | 510 | N | VAL | A | 64 | 27.869 | 0.424 | 13.876 | 1.00 | 14.70 | 7 | N |
| ATOM | 511 | CA | VAL | A | 64 | 27.905 | −0.973 | 14.276 | 1.00 | 14.29 | 6 | C |
| ATOM | 512 | C | VAL | A | 64 | 26.582 | −1.704 | 14.158 | 1.00 | 15.65 | 6 | C |
| ATOM | 513 | O | VAL | A | 64 | 26.571 | −2.958 | 14.145 | 1.00 | 14.07 | 8 | O |
| ATOM | 514 | CB | VAL | A | 64 | 28.444 | −1.149 | 15.714 | 1.00 | 14.63 | 6 | C |
| ATOM | 515 | CG1 | VAL | A | 64 | 29.886 | −0.655 | 15.761 | 1.00 | 14.17 | 6 | C |
| ATOM | 516 | CG2 | VAL | A | 64 | 27.554 | −0.445 | 16.721 | 1.00 | 16.21 | 6 | C |
| ATOM | 517 | N | GLU | A | 65 | 25.475 | −0.994 | 14.008 | 1.00 | 14.90 | 7 | N |
| ATOM | 518 | CA | GLU | A | 65 | 24.199 | −1.666 | 13.802 | 1.00 | 15.80 | 6 | C |
| ATOM | 519 | C | GLU | A | 65 | 24.235 | −2.532 | 12.543 | 1.00 | 15.72 | 6 | C |
| ATOM | 520 | O | GLU | A | 65 | 23.622 | −3.595 | 12.597 | 1.00 | 16.48 | 8 | O |
| ATOM | 521 | CB | GLU | A | 65 | 23.086 | −0.626 | 13.622 | 1.00 | 16.23 | 6 | C |
| ATOM | 522 | CG | GLU | A | 65 | 21.765 | −1.188 | 13.141 | 1.00 | 16.17 | 6 | C |
| ATOM | 523 | CD | GLU | A | 65 | 20.570 | −0.373 | 13.570 | 1.00 | 15.38 | 6 | C |
| ATOM | 524 | OE1 | GLU | A | 65 | 20.234 | 0.621 | 12.892 | 1.00 | 14.27 | 8 | O |
| ATOM | 525 | OE2 | GLU | A | 65 | 19.942 | −0.703 | 14.585 | 1.00 | 16.01 | 8 | O |
| ATOM | 526 | N | ASP | A | 66 | 24.895 | −2.146 | 11.458 | 1.00 | 15.62 | 7 | N |
| ATOM | 527 | CA | ASP | A | 66 | 24.838 | −2.969 | 10.237 | 1.00 | 15.25 | 6 | C |
| ATOM | 528 | C | ASP | A | 66 | 25.492 | −4.320 | 10.196 | 1.00 | 16.58 | 6 | C |
| ATOM | 529 | O | ASP | A | 66 | 24.912 | −5.303 | 9.696 | 1.00 | 14.96 | 8 | O |
| ATOM | 530 | CB | ASP | A | 66 | 25.212 | −2.071 | 9.041 | 1.00 | 15.64 | 6 | C |
| ATOM | 531 | CG | ASP | A | 66 | 24.239 | −0.901 | 8.977 | 1.00 | 15.18 | 6 | C |
| ATOM | 532 | OD1 | ASP | A | 66 | 23.014 | −1.156 | 9.034 | 1.00 | 15.88 | 8 | O |
| ATOM | 533 | OD2 | ASP | A | 66 | 24.701 | 0.244 | 8.883 | 1.00 | 15.87 | 8 | O |
| ATOM | 534 | N | PRO | A | 67 | 26.708 | −4.504 | 10.710 | 1.00 | 17.19 | 7 | N |
| ATOM | 535 | CA | PRO | A | 67 | 27.389 | −5.779 | 10.656 | 1.00 | 17.55 | 6 | C |
| ATOM | 536 | C | PRO | A | 67 | 26.848 | −6.912 | 11.484 | 1.00 | 16.75 | 6 | C |
| ATOM | 537 | O | PRO | A | 67 | 27.325 | −8.048 | 11.316 | 1.00 | 16.41 | 8 | O |
| ATOM | 538 | CB | PRO | A | 67 | 28.825 | −5.437 | 11.067 | 1.00 | 18.03 | 6 | C |
| ATOM | 539 | CG | PRO | A | 67 | 28.778 | −4.114 | 11.740 | 1.00 | 18.89 | 6 | C |
| ATOM | 540 | CD | PRO | A | 67 | 27.543 | −3.413 | 11.265 | 1.00 | 17.99 | 6 | C |
| ATOM | 541 | N | VAL | A | 68 | 25.907 | −6.759 | 12.410 | 1.00 | 16.46 | 7 | N |
| ATOM | 542 | CA | VAL | A | 68 | 25.408 | −7.821 | 13.237 | 1.00 | 17.04 | 6 | C |
| ATOM | 543 | C | VAL | A | 68 | 23.972 | −7.571 | 13.695 | 1.00 | 16.60 | 6 | C |
| ATOM | 544 | O | VAL | A | 68 | 23.536 | −6.436 | 13.815 | 1.00 | 17.81 | 8 | O |
| ATOM | 545 | CB | VAL | A | 68 | 26.150 | −7.958 | 14.603 | 1.00 | 19.25 | 6 | C |
| ATOM | 546 | CG1 | VAL | A | 68 | 25.925 | −9.360 | 15.181 | 1.00 | 18.50 | 6 | C |
| ATOM | 547 | CG2 | VAL | A | 68 | 27.631 | −7.654 | 14.614 | 1.00 | 19.81 | 6 | C |
| ATOM | 548 | N | LEU | A | 69 | 23.270 | −8.663 | 13.960 | 1.00 | 15.24 | 7 | N |
| ATOM | 549 | CA | LEU | A | 69 | 21.938 | −8.622 | 14.523 | 1.00 | 15.34 | 6 | C |
| ATOM | 550 | C | LEU | A | 69 | 22.014 | −9.403 | 15.847 | 1.00 | 16.19 | 6 | C |
| ATOM | 551 | O | LEU | A | 69 | 22.241 | −10.617 | 15.819 | 1.00 | 14.93 | 8 | O |
| ATOM | 552 | CB | LEU | A | 69 | 20.852 | −9.255 | 13.665 | 1.00 | 15.39 | 6 | C |
| ATOM | 553 | CG | LEU | A | 69 | 19.458 | −9.220 | 14.306 | 1.00 | 15.12 | 6 | C |
| ATOM | 554 | CD1 | LEU | A | 69 | 18.923 | −7.794 | 14.299 | 1.00 | 15.02 | 6 | C |
| ATOM | 555 | CD2 | LEU | A | 69 | 18.532 | −10.198 | 13.583 | 1.00 | 17.14 | 6 | C |
| ATOM | 556 | N | CYH | A | 70 | 21.863 | −8.714 | 16.971 | 1.00 | 16.02 | 7 | N |
| ATOM | 557 | CA | CYH | A | 70 | 21.891 | −9.395 | 18.261 | 1.00 | 16.43 | 6 | C |
| ATOM | 558 | C | CYH | A | 70 | 20.478 | −9.614 | 18.776 | 1.00 | 17.66 | 6 | C |
| ATOM | 559 | O | CYH | A | 70 | 19.735 | −8.644 | 18.902 | 1.00 | 18.21 | 8 | O |
| ATOM | 560 | CB | CYH | A | 70 | 22.638 | −8.521 | 19.285 | 1.00 | 14.65 | 6 | C |
| ATOM | 561 | SG | CYH | A | 70 | 24.324 | −8.245 | 18.730 | 1.00 | 15.51 | 16 | S |
| ATOM | 562 | N | THR | A | 71 | 20.122 | −10.861 | 19.074 | 1.00 | 19.11 | 7 | N |
| ATOM | 563 | CA | THR | A | 71 | 18.795 | −11.150 | 19.602 | 1.00 | 20.68 | 6 | C |
| ATOM | 564 | C | THR | A | 71 | 18.929 | −11.737 | 20.998 | 1.00 | 21.80 | 6 | C |
| ATOM | 565 | O | THR | A | 71 | 20.036 | −11.832 | 21.542 | 1.00 | 23.70 | 8 | O |
| ATOM | 566 | CB | THR | A | 71 | 18.003 | −12.115 | 18.691 | 1.00 | 21.33 | 6 | C |
| ATOM | 567 | OG1 | THR | A | 71 | 18.451 | −13.462 | 18.910 | 1.00 | 21.84 | 8 | O |
| ATOM | 568 | CG2 | THR | A | 71 | 18.198 | −11.785 | 17.218 | 1.00 | 20.37 | 6 | C |
| ATOM | 569 | N | SER | A | 72 | 17.830 | −12.158 | 21.626 | 1.00 | 23.10 | 7 | N |
| ATOM | 570 | CA | SER | A | 72 | 17.911 | −12.762 | 22.953 | 1.00 | 24.29 | 6 | C |
| ATOM | 571 | C | SER | A | 72 | 18.323 | −14.227 | 22.889 | 1.00 | 24.83 | 6 | C |
| ATOM | 572 | O | SER | A | 72 | 18.585 | −14.843 | 23.926 | 1.00 | 26.58 | 8 | O |
| ATOM | 573 | CB | SER | A | 72 | 16.528 | −12.722 | 23.632 | 1.00 | 23.63 | 6 | C |
| ATOM | 574 | OG | SER | A | 72 | 15.591 | −13.337 | 22.750 | 1.00 | 24.62 | 8 | O |
| ATOM | 575 | N | ARG | A | 73 | 18.346 | −14.808 | 21.698 | 1.00 | 25.24 | 7 | N |
| ATOM | 576 | CA | ARG | A | 73 | 18.684 | −16.207 | 21.513 | 1.00 | 25.43 | 6 | C |
| ATOM | 577 | C | ARG | A | 73 | 19.994 | −16.446 | 20.789 | 1.00 | 23.96 | 6 | C |
| ATOM | 578 | O | ARG | A | 73 | 20.616 | −17.492 | 20.987 | 1.00 | 24.45 | 8 | O |
| ATOM | 579 | CB | ARG | A | 73 | 17.583 | −16.898 | 20.688 | 1.00 | 27.43 | 6 | C |
| ATOM | 580 | CG | ARG | A | 73 | 16.160 | −16.600 | 21.097 | 1.00 | 29.59 | 6 | C |
| ATOM | 581 | CD | ARG | A | 73 | 15.552 | −17.613 | 22.045 | 1.00 | 29.78 | 6 | C |
| ATOM | 582 | NE | ARG | A | 73 | 14.244 | −17.165 | 22.502 | 1.00 | 31.19 | 7 | N |
| ATOM | 583 | CZ | ARG | A | 73 | 13.059 | −17.406 | 21.966 | 1.00 | 31.65 | 6 | C |
| ATOM | 584 | NH1 | ARG | A | 73 | 12.927 | −18.142 | 20.874 | 1.00 | 31.37 | 7 | N |
| ATOM | 585 | NH2 | ARG | A | 73 | 11.973 | −16.895 | 22.549 | 1.00 | 32.48 | 7 | N |
| ATOM | 586 | N | CYH | A | 74 | 20.425 | −15.539 | 19.927 | 1.00 | 23.06 | 7 | N |
| ATOM | 587 | CA | CYH | A | 74 | 21.658 | −15.679 | 19.176 | 1.00 | 21.25 | 6 | C |
| ATOM | 588 | C | CYH | A | 74 | 22.055 | −14.367 | 18.506 | 1.00 | 19.75 | 6 | C |
| ATOM | 589 | O | CYH | A | 74 | 21.308 | −13.400 | 18.537 | 1.00 | 20.11 | 8 | O |
| ATOM | 590 | CB | CYH | A | 74 | 21.503 | −16.712 | 18.041 | 1.00 | 22.29 | 6 | C |
| ATOM | 591 | SG | CYH | A | 74 | 20.311 | −16.169 | 16.777 | 1.00 | 22.85 | 16 | S |
| ATOM | 592 | N | ALA | A | 75 | 23.247 | −14.370 | 17.917 | 1.00 | 18.42 | 7 | N |
| ATOM | 593 | CA | ALA | A | 75 | 23.754 | −13.227 | 17.187 | 1.00 | 18.43 | 6 | C |
| ATOM | 594 | C | ALA | A | 75 | 24.026 | −13.742 | 15.758 | 1.00 | 18.09 | 6 | C |
| ATOM | 595 | O | ALA | A | 75 | 24.549 | −14.841 | 15.567 | 1.00 | 18.60 | 8 | O |
| ATOM | 596 | CB | ALA | A | 75 | 25.007 | −12.585 | 17.724 | 1.00 | 18.72 | 6 | C |
| ATOM | 597 | N | ILE | A | 76 | 23.657 | −12.923 | 14.784 | 1.00 | 16.76 | 7 | N |
| ATOM | 598 | CA | ILE | A | 76 | 23.860 | −13.279 | 13.382 | 1.00 | 16.43 | 6 | C |
| ATOM | 599 | C | ILE | A | 76 | 24.745 | −12.208 | 12.744 | 1.00 | 17.72 | 6 | C |
| ATOM | 600 | O | ILE | A | 76 | 24.313 | −11.055 | 12.677 | 1.00 | 17.31 | 8 | O |
| ATOM | 601 | CB | ILE | A | 76 | 22.533 | −13.300 | 12.603 | 1.00 | 17.55 | 6 | C |
| ATOM | 602 | CG1 | ILE | A | 76 | 21.469 | −14.169 | 13.278 | 1.00 | 17.48 | 6 | C |
| ATOM | 603 | CG2 | ILE | A | 76 | 22.729 | −13.789 | 11.169 | 1.00 | 17.08 | 6 | C |
| ATOM | 604 | CD1 | ILE | A | 76 | 20.494 | −13.409 | 14.160 | 1.00 | 19.45 | 6 | C |
| ATOM | 605 | N | ILE | A | 77 | 25.940 | −12.584 | 12.332 | 1.00 | 16.21 | 7 | N |
| ATOM | 606 | CA | ILE | A | 77 | 26.852 | −11.670 | 11.666 | 1.00 | 16.85 | 6 | C |
| ATOM | 607 | C | ILE | A | 77 | 26.269 | −11.512 | 10.258 | 1.00 | 17.30 | 6 | C |
| ATOM | 608 | O | ILE | A | 77 | 25.934 | −12.525 | 9.639 | 1.00 | 16.99 | 8 | O |
| ATOM | 609 | CB | ILE | A | 77 | 28.268 | −12.246 | 11.564 | 1.00 | 18.20 | 6 | C |
| ATOM | 610 | CG1 | ILE | A | 77 | 28.798 | −12.576 | 12.964 | 1.00 | 19.73 | 6 | C |
| ATOM | 611 | CG2 | ILE | A | 77 | 29.233 | −11.305 | 10.852 | 1.00 | 18.59 | 6 | C |
| ATOM | 612 | CD1 | ILE | A | 77 | 28.896 | −11.387 | 13.897 | 1.00 | 20.69 | 6 | C |
| ATOM | 613 | N | THR | A | 78 | 26.115 | −10.274 | 9.825 | 1.00 | 17.26 | 7 | N |
| ATOM | 614 | CA | THR | A | 78 | 25.504 | −10.045 | 8.511 | 1.00 | 16.84 | 6 | C |
| ATOM | 615 | C | THR | A | 78 | 26.521 | −10.107 | 7.396 | 1.00 | 17.30 | 6 | C |
| ATOM | 616 | O | THR | A | 78 | 27.604 | −10.700 | 7.503 | 1.00 | 17.35 | 8 | O |
| ATOM | 617 | CB | THR | A | 78 | 24.741 | −8.707 | 8.556 | 1.00 | 16.17 | 6 | C |
| ATOM | 618 | OG1 | THR | A | 78 | 25.676 | −7.632 | 8.705 | 1.00 | 16.64 | 8 | O |
| ATOM | 619 | CG2 | THR | A | 78 | 23.733 | −8.681 | 9.696 | 1.00 | 16.57 | 6 | C |
| ATOM | 620 | N | ARG | A | 79 | 26.175 | −9.542 | 6.239 | 1.00 | 17.52 | 7 | N |
| ATOM | 621 | CA | ARG | A | 79 | 27.022 | −9.513 | 5.061 | 1.00 | 17.69 | 6 | C |
| ATOM | 622 | C | ARG | A | 79 | 26.664 | −8.249 | 4.286 | 1.00 | 17.66 | 6 | C |
| ATOM | 623 | O | ARG | A | 79 | 25.827 | −8.247 | 3.378 | 1.00 | 16.27 | 8 | O |
| ATOM | 624 | CB | ARG | A | 79 | 26.806 | −10.792 | 4.232 | 1.00 | 19.26 | 6 | C |
| ATOM | 625 | CG | ARG | A | 79 | 27.821 | −10.945 | 3.103 | 1.00 | 20.03 | 6 | C |
| ATOM | 626 | CD | ARG | A | 79 | 27.965 | −12.413 | 2.676 | 1.00 | 22.06 | 6 | C |
| ATOM | 627 | NE | ARG | A | 79 | 28.917 | −12.520 | 1.569 | 1.00 | 23.24 | 7 | N |
| ATOM | 628 | CZ | ARG | A | 79 | 28.640 | −12.296 | 0.289 | 1.00 | 25.29 | 6 | C |
| ATOM | 629 | NH1 | ARG | A | 79 | 27.414 | −11.951 | −0.080 | 1.00 | 25.80 | 7 | N |
| ATOM | 630 | NH2 | ARG | A | 79 | 29.593 | −12.420 | −0.639 | 1.00 | 25.28 | 7 | N |
| ATOM | 631 | N | PRO | A | 80 | 27.241 | −7.127 | 4.695 | 1.00 | 17.87 | 7 | N |
| ATOM | 632 | CA | PRO | A | 80 | 26.947 | −5.831 | 4.108 | 1.00 | 18.57 | 6 | C |
| ATOM | 633 | C | PRO | A | 80 | 26.952 | −5.786 | 2.598 | 1.00 | 18.02 | 6 | C |
| ATOM | 634 | O | PRO | A | 80 | 27.813 | −6.338 | 1.916 | 1.00 | 18.53 | 8 | O |
| ATOM | 635 | CB | PRO | A | 80 | 27.955 | −4.884 | 4.741 | 1.00 | 18.38 | 6 | C |
| ATOM | 636 | CG | PRO | A | 80 | 28.225 | −5.505 | 6.081 | 1.00 | 18.76 | 6 | C |
| ATOM | 637 | CD | PRO | A | 80 | 28.213 | −6.999 | 5.812 | 1.00 | 18.81 | 6 | C |
| ATOM | 638 | N | GLY | A | 81 | 25.940 | −5.090 | 2.064 | 1.00 | 18.24 | 7 | N |
| ATOM | 639 | CA | GLY | A | 81 | 25.824 | −4.938 | 0.611 | 1.00 | 19.26 | 6 | C |
| ATOM | 640 | C | GLY | A | 81 | 26.864 | −3.923 | 0.138 | 1.00 | 19.95 | 6 | C |
| ATOM | 641 | O | GLY | A | 81 | 27.348 | −4.018 | −0.980 | 1.00 | 19.91 | 8 | O |
| ATOM | 642 | N | ALA | A | 82 | 27.186 | −2.928 | 0.966 | 1.00 | 20.24 | 7 | N |
| ATOM | 643 | CA | ALA | A | 82 | 28.157 | −1.914 | 0.569 | 1.00 | 20.36 | 6 | C |
| ATOM | 644 | C | ALA | A | 82 | 29.565 | −2.502 | 0.540 | 1.00 | 21.45 | 6 | C |
| ATOM | 645 | O | ALA | A | 82 | 30.014 | −2.923 | 1.600 | 1.00 | 20.63 | 8 | O |
| ATOM | 646 | CB | ALA | A | 82 | 28.095 | −0.731 | 1.522 | 1.00 | 19.67 | 6 | C |
| ATOM | 647 | N | GLU | A | 83 | 30.222 | −2.520 | −0.608 | 1.00 | 21.74 | 7 | N |
| ATOM | 648 | CA | GLU | A | 83 | 31.561 | −3.055 | −0.784 | 1.00 | 21.33 | 6 | C |
| ATOM | 649 | C | GLU | A | 83 | 32.571 | −2.633 | 0.278 | 1.00 | 21.21 | 6 | C |
| ATOM | 650 | O | GLU | A | 83 | 33.281 | −3.465 | 0.855 | 1.00 | 20.94 | 8 | O |
| ATOM | 651 | CB | GLU | A | 83 | 32.125 | −2.580 | −2.149 | 1.00 | 22.51 | 6 | C |
| ATOM | 652 | CG | GLU | A | 83 | 31.752 | −4.104 | −3.103 | 0.00 | 20.00 | 6 | C |
| ATOM | 653 | CD | GLU | A | 83 | 32.220 | −5.470 | −2.640 | 0.00 | 20.00 | 6 | C |
| ATOM | 654 | OE1 | GLU | A | 83 | 31.406 | −6.165 | −1.981 | 0.00 | 20.00 | 8 | O |
| ATOM | 655 | OE2 | GLU | A | 83 | 33.379 | −5.833 | −2.924 | 0.00 | 20.00 | 8 | O |
| ATOM | 656 | N | SER | A | 84 | 32.667 | −1.339 | 0.553 | 1.00 | 20.10 | 7 | N |
| ATOM | 657 | CA | SER | A | 84 | 33.579 | −0.795 | 1.551 | 1.00 | 19.82 | 6 | C |
| ATOM | 658 | C | SER | A | 84 | 33.262 | −1.147 | 2.995 | 1.00 | 19.64 | 6 | C |
| ATOM | 659 | O | SER | A | 84 | 34.099 | −0.914 | 3.889 | 1.00 | 19.24 | 8 | O |
| ATOM | 660 | CB | SER | A | 84 | 33.613 | 0.743 | 1.458 | 1.00 | 20.01 | 6 | C |
| ATOM | 661 | OG | SER | A | 84 | 32.328 | 1.267 | 1.805 | 1.00 | 19.32 | 8 | O |
| ATOM | 662 | N | ARG | A | 85 | 32.090 | −1.699 | 3.306 | 1.00 | 18.53 | 7 | N |
| ATOM | 663 | CA | ARG | A | 85 | 31.707 | −2.050 | 4.652 | 1.00 | 17.97 | 6 | C |
| ATOM | 664 | C | ARG | A | 85 | 31.614 | −3.551 | 4.904 | 1.00 | 18.87 | 6 | C |
| ATOM | 665 | O | ARG | A | 85 | 31.405 | −3.923 | 6.061 | 1.00 | 16.55 | 8 | O |
| ATOM | 666 | CB | ARG | A | 85 | 30.326 | −1.415 | 4.949 | 1.00 | 18.53 | 6 | C |
| ATOM | 667 | CG | ARG | A | 85 | 30.320 | 0.100 | 4.769 | 1.00 | 17.62 | 6 | C |
| ATOM | 668 | CD | ARG | A | 85 | 29.192 | 0.739 | 5.567 | 1.00 | 17.33 | 6 | C |
| ATOM | 669 | NE | ARG | A | 85 | 29.260 | 0.309 | 6.969 | 1.00 | 16.97 | 7 | N |
| ATOM | 670 | CZ | ARG | A | 85 | 28.255 | 0.441 | 7.825 | 1.00 | 16.26 | 6 | C |
| ATOM | 671 | NH1 | ARG | A | 85 | 27.106 | 0.990 | 7.459 | 1.00 | 16.16 | 7 | N |
| ATOM | 672 | NH2 | ARG | A | 85 | 28.405 | 0.011 | 9.068 | 1.00 | 14.30 | 7 | N |
| ATOM | 673 | N | ARG | A | 86 | 31.781 | −4.411 | 3.891 | 1.00 | 19.50 | 7 | N |
| ATOM | 674 | CA | ARG | A | 86 | 31.679 | −5.846 | 4.115 | 1.00 | 21.87 | 6 | C |
| ATOM | 675 | C | ARG | A | 86 | 32.661 | −6.354 | 5.165 | 1.00 | 21.45 | 6 | C |
| ATOM | 676 | O | ARG | A | 86 | 32.281 | −7.148 | 6.019 | 1.00 | 20.90 | 8 | O |
| ATOM | 677 | CB | ARG | A | 86 | 31.828 | −6.690 | 2.842 | 1.00 | 24.23 | 6 | C |
| ATOM | 678 | CG | ARG | A | 86 | 31.336 | −8.124 | 3.062 | 1.00 | 26.63 | 6 | C |
| ATOM | 679 | CD | ARG | A | 86 | 31.027 | −8.797 | 1.743 | 1.00 | 30.30 | 6 | C |
| ATOM | 680 | NE | ARG | A | 86 | 29.892 | −8.193 | 1.059 | 1.00 | 33.30 | 7 | N |
| ATOM | 681 | CZ | ARG | A | 86 | 29.743 | −8.164 | −0.261 | 1.00 | 35.57 | 6 | C |
| ATOM | 682 | NH1 | ARG | A | 86 | 30.656 | −8.701 | −1.070 | 1.00 | 36.41 | 7 | N |
| ATOM | 683 | NH2 | ARG | A | 86 | 28.678 | −7.590 | −0.799 | 1.00 | 36.55 | 7 | N |
| ATOM | 684 | N | GLY | A | 87 | 33.894 | −5.877 | 5.167 | 1.00 | 20.95 | 7 | N |
| ATOM | 685 | CA | GLY | A | 87 | 34.929 | −6.260 | 6.100 | 1.00 | 20.84 | 6 | C |
| ATOM | 686 | C | GLY | A | 87 | 34.702 | −5.854 | 7.537 | 1.00 | 20.72 | 6 | C |
| ATOM | 687 | O | GLY | A | 87 | 35.445 | −6.271 | 8.442 | 1.00 | 20.75 | 8 | O |
| ATOM | 688 | N | GLU | A | 88 | 33.690 | −5.034 | 7.844 | 1.00 | 18.97 | 7 | N |
| ATOM | 689 | CA | GLU | A | 88 | 33.375 | −4.626 | 9.187 | 1.00 | 18.67 | 6 | C |
| ATOM | 690 | C | GLU | A | 88 | 32.905 | −5.790 | 10.055 | 1.00 | 18.56 | 6 | C |
| ATOM | 691 | O | GLU | A | 88 | 32.980 | −5.718 | 11.288 | 1.00 | 17.95 | 8 | O |
| ATOM | 692 | CB | GLU | A | 88 | 32.266 | −3.556 | 9.233 | 1.00 | 17.87 | 6 | C |
| ATOM | 693 | CG | GLU | A | 88 | 32.795 | −2.260 | 8.621 | 1.00 | 17.40 | 6 | C |
| ATOM | 694 | CD | GLU | A | 88 | 31.793 | −1.125 | 8.686 | 1.00 | 17.16 | 6 | C |
| ATOM | 695 | OE1 | GLU | A | 88 | 30.889 | −1.179 | 9.545 | 1.00 | 17.99 | 8 | O |
| ATOM | 696 | OE2 | GLU | A | 88 | 31.933 | −0.196 | 7.857 | 1.00 | 16.44 | 8 | O |
| ATOM | 697 | N | THR | A | 89 | 32.423 | −6.869 | 9.434 | 1.00 | 17.66 | 7 | N |
| ATOM | 698 | CA | THR | A | 89 | 31.993 | −8.029 | 10.205 | 1.00 | 18.74 | 6 | C |
| ATOM | 699 | C | THR | A | 89 | 33.178 | −8.669 | 10.930 | 1.00 | 19.66 | 6 | C |
| ATOM | 700 | O | THR | A | 89 | 33.039 | −9.187 | 12.035 | 1.00 | 19.57 | 8 | O |
| ATOM | 701 | CB | THR | A | 89 | 31.333 | −9.096 | 9.295 | 1.00 | 19.39 | 6 | C |
| ATOM | 702 | OG1 | THR | A | 89 | 32.214 | −9.432 | 8.234 | 1.00 | 19.75 | 8 | O |
| ATOM | 703 | CG2 | THR | A | 89 | 30.031 | −8.523 | 8.741 | 1.00 | 19.11 | 6 | C |
| ATOM | 704 | N | GLU | A | 90 | 34.353 | −8.641 | 10.300 | 1.00 | 19.95 | 7 | N |
| ATOM | 705 | CA | GLU | A | 90 | 35.557 | −9.234 | 10.849 | 1.00 | 21.86 | 6 | C |
| ATOM | 706 | C | GLU | A | 90 | 36.080 | −8.557 | 12.110 | 1.00 | 21.25 | 6 | C |
| ATOM | 707 | O | GLU | A | 90 | 36.564 | −9.251 | 13.012 | 1.00 | 21.17 | 8 | O |
| ATOM | 708 | CB | GLU | A | 90 | 36.678 | −9.243 | 9.793 | 1.00 | 23.24 | 6 | C |
| ATOM | 709 | CG | GLU | A | 90 | 36.320 | −9.988 | 8.519 | 1.00 | 27.58 | 6 | C |
| ATOM | 710 | CD | GLU | A | 90 | 36.220 | −11.484 | 8.683 | 1.00 | 30.23 | 6 | C |
| ATOM | 711 | OE1 | GLU | A | 90 | 35.249 | −12.079 | 8.171 | 1.00 | 32.62 | 8 | O |
| ATOM | 712 | OE2 | GLU | A | 90 | 37.096 | −12.101 | 9.328 | 1.00 | 31.88 | 8 | O |
| ATOM | 713 | N | ILE | A | 91 | 35.986 | −7.239 | 12.176 | 1.00 | 20.41 | 7 | N |
| ATOM | 714 | CA | ILE | A | 91 | 36.482 | −6.492 | 13.329 | 1.00 | 20.23 | 6 | C |
| ATOM | 715 | C | ILE | A | 91 | 35.504 | −6.471 | 14.481 | 1.00 | 20.38 | 6 | C |
| ATOM | 716 | O | ILE | A | 91 | 35.845 | −6.016 | 15.585 | 1.00 | 21.64 | 8 | O |
| ATOM | 717 | CB | ILE | A | 91 | 36.910 | −5.071 | 12.909 | 1.00 | 20.22 | 6 | C |
| ATOM | 718 | CG1 | ILE | A | 91 | 35.781 | −4.306 | 12.217 | 1.00 | 19.30 | 6 | C |
| ATOM | 719 | CG2 | ILE | A | 91 | 38.126 | −5.149 | 11.995 | 1.00 | 19.70 | 6 | C |
| ATOM | 720 | CD1 | ILE | A | 91 | 36.123 | −2.832 | 12.060 | 1.00 | 18.76 | 6 | C |
| ATOM | 721 | N | ILE | A | 92 | 34.287 | −6.974 | 14.300 | 1.00 | 20.71 | 7 | N |
| ATOM | 722 | CA | ILE | A | 92 | 33.317 | −7.020 | 15.386 | 1.00 | 20.29 | 6 | C |
| ATOM | 723 | C | ILE | A | 92 | 33.054 | −8.437 | 15.868 | 1.00 | 20.58 | 6 | C |
| ATOM | 724 | O | ILE | A | 92 | 32.453 | −8.612 | 16.933 | 1.00 | 20.59 | 8 | O |
| ATOM | 725 | CB | ILE | A | 92 | 32.003 | −6.331 | 15.019 | 1.00 | 21.38 | 6 | C |
| ATOM | 726 | CG1 | ILE | A | 92 | 31.274 | −5.890 | 16.296 | 1.00 | 21.52 | 6 | C |
| ATOM | 727 | CG2 | ILE | A | 92 | 31.131 | −7.253 | 14.174 | 1.00 | 21.71 | 6 | C |
| ATOM | 728 | CD1 | ILE | A | 92 | 30.238 | −4.819 | 16.070 | 1.00 | 21.61 | 6 | C |
| ATOM | 729 | N | GLU | A | 93 | 33.497 | −9.439 | 15.111 | 1.00 | 21.73 | 7 | N |
| ATOM | 730 | CA | GLU | A | 93 | 33.273 | −10.827 | 15.453 | 1.00 | 22.68 | 6 | C |
| ATOM | 731 | C | GLU | A | 93 | 33.758 | −11.261 | 16.828 | 1.00 | 21.42 | 6 | C |
| ATOM | 732 | O | GLU | A | 93 | 33.052 | −12.008 | 17.499 | 1.00 | 20.00 | 8 | O |
| ATOM | 733 | CB | GLU | A | 93 | 33.843 | −11.780 | 14.385 | 1.00 | 26.54 | 6 | C |
| ATOM | 734 | CG | GLU | A | 93 | 33.244 | −13.167 | 14.576 | 1.00 | 31.50 | 6 | C |
| ATOM | 735 | CD | GLU | A | 93 | 33.020 | −14.001 | 13.343 | 1.00 | 34.57 | 6 | C |
| ATOM | 736 | OE1 | GLU | A | 93 | 32.747 | −13.441 | 12.254 | 1.00 | 36.36 | 8 | O |
| ATOM | 737 | OE2 | GLU | A | 93 | 33.118 | −15.254 | 13.475 | 1.00 | 36.18 | 8 | O |
| ATOM | 738 | N | GLU | A | 94 | 34.923 | −10.808 | 17.272 | 1.00 | 19.91 | 7 | N |
| ATOM | 739 | CA | GLU | A | 94 | 35.426 | −11.223 | 18.592 | 1.00 | 18.82 | 6 | C |
| ATOM | 740 | C | GLU | A | 94 | 34.522 | −10.782 | 19.725 | 1.00 | 18.38 | 6 | C |
| ATOM | 741 | O | GLU | A | 94 | 34.227 | −11.515 | 20.677 | 1.00 | 16.83 | 8 | O |
| ATOM | 742 | CB | GLU | A | 94 | 36.863 | −10.714 | 18.720 | 1.00 | 20.85 | 6 | C |
| ATOM | 743 | CG | GLU | A | 94 | 37.463 | −11.044 | 20.147 | 0.00 | 20.00 | 6 | C |
| ATOM | 744 | CD | GLU | A | 94 | 38.919 | −10.694 | 20.332 | 0.00 | 20.00 | 6 | C |
| ATOM | 745 | OE1 | GLU | A | 94 | 39.581 | −10.140 | 19.442 | 0.00 | 20.00 | 8 | O |
| ATOM | 746 | OE2 | GLU | A | 94 | 39.456 | −10.979 | 21.433 | 0.00 | 20.00 | 8 | O |
| ATOM | 747 | N | THR | A | 95 | 34.015 | −9.547 | 19.644 | 1.00 | 17.39 | 7 | N |
| ATOM | 748 | CA | THR | A | 95 | 33.103 | −9.040 | 20.672 | 1.00 | 17.71 | 6 | C |
| ATOM | 749 | C | THR | A | 95 | 31.818 | −9.846 | 20.705 | 1.00 | 17.11 | 6 | C |
| ATOM | 750 | O | THR | A | 95 | 31.323 | −10.205 | 21.772 | 1.00 | 17.49 | 8 | O |
| ATOM | 751 | CB | THR | A | 95 | 32.765 | −7.562 | 20.358 | 1.00 | 17.77 | 6 | C |
| ATOM | 752 | OG1 | THR | A | 95 | 33.963 | −6.790 | 20.350 | 1.00 | 17.71 | 8 | O |
| ATOM | 753 | CG2 | THR | A | 95 | 31.783 | −6.967 | 21.330 | 1.00 | 17.04 | 6 | C |
| ATOM | 754 | N | VAL | A | 96 | 31.247 | −10.107 | 19.519 | 1.00 | 18.28 | 7 | N |
| ATOM | 755 | CA | VAL | A | 96 | 29.983 | −10.857 | 19.463 | 1.00 | 19.18 | 6 | C |
| ATOM | 756 | C | VAL | A | 96 | 30.167 | −12.246 | 20.061 | 1.00 | 19.16 | 6 | C |
| ATOM | 757 | O | VAL | A | 96 | 29.355 | −12.752 | 20.834 | 1.00 | 15.65 | 8 | O |
| ATOM | 758 | CB | VAL | A | 96 | 29.460 | −10.929 | 18.016 | 1.00 | 19.21 | 6 | C |
| ATOM | 759 | CG1 | VAL | A | 96 | 28.274 | −11.872 | 17.878 | 1.00 | 19.66 | 6 | C |
| ATOM | 760 | CG2 | VAL | A | 96 | 29.061 | −9.520 | 17.560 | 1.00 | 20.45 | 6 | C |
| ATOM | 761 | N | GLN | A | 97 | 31.273 | −12.875 | 19.670 | 1.00 | 20.21 | 7 | N |
| ATOM | 762 | CA | GLN | A | 97 | 31.615 | −14.221 | 20.139 | 1.00 | 20.83 | 6 | C |
| ATOM | 763 | C | GLN | A | 97 | 31.714 | −14.270 | 21.654 | 1.00 | 19.10 | 6 | C |
| ATOM | 764 | O | GLN | A | 97 | 31.274 | −15.215 | 22.319 | 1.00 | 18.47 | 8 | O |
| ATOM | 765 | CB | GLN | A | 97 | 32.926 | −14.646 | 19.460 | 1.00 | 24.02 | 6 | C |
| ATOM | 766 | CG | GLN | A | 97 | 33.122 | −16.154 | 19.440 | 1.00 | 28.94 | 6 | C |
| ATOM | 767 | CD | GLN | A | 97 | 32.161 | −16.861 | 18.501 | 1.00 | 30.73 | 6 | C |
| ATOM | 768 | OE1 | GLN | A | 97 | 31.385 | −17.681 | 18.991 | 1.00 | 33.19 | 8 | O |
| ATOM | 769 | NE2 | GLN | A | 97 | 32.208 | −16.555 | 17.209 | 1.00 | 32.73 | 7 | N |
| ATOM | 770 | N | ARG | A | 98 | 32.238 | −13.211 | 22.274 | 1.00 | 18.73 | 7 | N |
| ATOM | 771 | CA | ARG | A | 98 | 32.347 | −13.142 | 23.731 | 1.00 | 16.98 | 6 | C |
| ATOM | 772 | C | ARG | A | 98 | 30.990 | −13.034 | 24.409 | 1.00 | 16.40 | 6 | C |
| ATOM | 773 | O | ARG | A | 98 | 30.776 | −13.555 | 25.487 | 1.00 | 16.31 | 8 | O |
| ATOM | 774 | CB | ARG | A | 98 | 33.232 | −11.962 | 24.150 | 1.00 | 18.64 | 6 | C |
| ATOM | 775 | CG | ARG | A | 98 | 33.605 | −12.024 | 25.630 | 1.00 | 21.41 | 6 | C |
| ATOM | 776 | CD | ARG | A | 98 | 34.641 | −10.986 | 25.990 | 1.00 | 23.93 | 6 | C |
| ATOM | 777 | NE | ARG | A | 98 | 35.437 | −10.503 | 24.865 | 1.00 | 26.86 | 7 | N |
| ATOM | 778 | CZ | ARG | A | 98 | 35.274 | −9.263 | 24.385 | 1.00 | 26.97 | 6 | C |
| ATOM | 779 | NH1 | ARG | A | 98 | 34.391 | −8.428 | 24.907 | 1.00 | 29.13 | 7 | N |
| ATOM | 780 | NH2 | ARG | A | 98 | 36.039 | −8.918 | 23.375 | 1.00 | 29.19 | 7 | N |
| ATOM | 781 | N | PHE | A | 99 | 30.015 | −12.364 | 23.778 | 1.00 | 15.60 | 7 | N |
| ATOM | 782 | CA | PHE | A | 99 | 28.686 | −12.247 | 24.364 | 1.00 | 16.73 | 6 | C |
| ATOM | 783 | C | PHE | A | 99 | 27.733 | −13.396 | 24.062 | 1.00 | 17.36 | 6 | C |
| ATOM | 784 | O | PHE | A | 99 | 26.732 | −13.618 | 24.763 | 1.00 | 18.31 | 8 | O |
| ATOM | 785 | CB | PHE | A | 99 | 27.998 | −10.976 | 23.833 | 1.00 | 16.35 | 6 | C |
| ATOM | 786 | CG | PHE | A | 99 | 28.443 | −9.737 | 24.567 | 1.00 | 16.90 | 6 | C |
| ATOM | 787 | CD1 | PHE | A | 99 | 29.297 | −8.828 | 23.977 | 1.00 | 18.51 | 6 | C |
| ATOM | 788 | CD2 | PHE | A | 99 | 27.990 | −9.512 | 25.851 | 1.00 | 16.40 | 6 | C |
| ATOM | 789 | CE1 | PHE | A | 99 | 29.690 | −7.693 | 24.677 | 1.00 | 18.49 | 6 | C |
| ATOM | 790 | CE2 | PHE | A | 99 | 28.376 | −8.387 | 26.560 | 1.00 | 17.86 | 6 | C |
| ATOM | 791 | CZ | PHE | A | 99 | 29.230 | −7.480 | 25.955 | 1.00 | 18.42 | 6 | C |
| ATOM | 792 | N | TYR | A | 100 | 28.005 | −14.120 | 22.988 | 1.00 | 18.68 | 7 | N |
| ATOM | 793 | CA | TYR | A | 100 | 27.171 | −15.221 | 22.507 | 1.00 | 19.60 | 6 | C |
| ATOM | 794 | C | TYR | A | 100 | 27.949 | −16.493 | 22.227 | 1.00 | 20.98 | 6 | C |
| ATOM | 795 | O | TYR | A | 100 | 28.073 | −16.985 | 21.092 | 1.00 | 20.59 | 8 | O |
| ATOM | 796 | CB | TYR | A | 100 | 26.567 | −14.706 | 21.168 | 1.00 | 18.50 | 6 | C |
| ATOM | 797 | CG | TYR | A | 100 | 25.488 | −13.654 | 21.296 | 1.00 | 18.50 | 6 | C |
| ATOM | 798 | CD1 | TYR | A | 100 | 25.737 | −12.292 | 21.124 | 1.00 | 18.99 | 6 | C |
| ATOM | 799 | CD2 | TYR | A | 100 | 24.190 | −14.039 | 21.597 | 1.00 | 18.92 | 6 | C |
| ATOM | 800 | CE1 | TYR | A | 100 | 24.717 | −11.364 | 21.253 | 1.00 | 18.06 | 6 | C |
| ATOM | 801 | CE2 | TYR | A | 100 | 23.163 | −13.120 | 21.730 | 1.00 | 19.65 | 6 | C |
| ATOM | 802 | CZ | TYR | A | 100 | 23.436 | −11.777 | 21.553 | 1.00 | 18.58 | 6 | C |
| ATOM | 803 | OH | TYR | A | 100 | 22.420 | −10.868 | 21.684 | 1.00 | 18.53 | 8 | O |
| ATOM | 804 | N | PRO | A | 101 | 28.528 | −17.088 | 23.261 | 1.00 | 21.64 | 7 | N |
| ATOM | 805 | CA | PRO | A | 101 | 29.336 | −18.293 | 23.093 | 1.00 | 22.66 | 6 | C |
| ATOM | 806 | C | PRO | A | 101 | 28.505 | −19.455 | 22.589 | 1.00 | 22.95 | 6 | C |
| ATOM | 807 | O | PRO | A | 101 | 27.466 | −19.815 | 23.126 | 1.00 | 24.97 | 8 | O |
| ATOM | 808 | CB | PRO | A | 101 | 29.978 | −18.519 | 24.456 | 1.00 | 22.79 | 6 | C |
| ATOM | 809 | CG | PRO | A | 101 | 29.126 | −17.785 | 25.422 | 1.00 | 22.22 | 6 | C |
| ATOM | 810 | CD | PRO | A | 101 | 28.492 | −16.654 | 24.672 | 1.00 | 22.49 | 6 | C |
| ATOM | 811 | N | GLY | A | 102 | 28.953 | −20.029 | 21.477 | 1.00 | 24.57 | 7 | N |
| ATOM | 812 | CA | GLY | A | 102 | 28.276 | −21.149 | 20.835 | 1.00 | 24.66 | 6 | C |
| ATOM | 813 | C | GLY | A | 102 | 26.934 | −20.722 | 20.271 | 1.00 | 25.38 | 6 | C |
| ATOM | 814 | O | GLY | A | 102 | 26.033 | −21.556 | 20.095 | 1.00 | 25.75 | 8 | O |
| ATOM | 815 | N | LYS | A | 103 | 26.747 | −19.424 | 19.979 | 1.00 | 24.54 | 7 | N |
| ATOM | 816 | CA | LYS | A | 103 | 25.472 | −18.951 | 19.456 | 1.00 | 24.39 | 6 | C |
| ATOM | 817 | C | LYS | A | 103 | 25.623 | −17.874 | 18.384 | 1.00 | 23.38 | 6 | C |
| ATOM | 818 | O | LYS | A | 103 | 24.801 | −16.954 | 18.331 | 1.00 | 22.41 | 8 | O |
| ATOM | 819 | CB | LYS | A | 103 | 24.593 | −18.404 | 20.582 | 1.00 | 26.13 | 6 | C |
| ATOM | 820 | CG | LYS | A | 103 | 23.945 | −19.436 | 21.494 | 1.00 | 27.62 | 6 | C |
| ATOM | 821 | CD | LYS | A | 103 | 23.110 | −18.732 | 22.549 | 1.00 | 30.03 | 6 | C |
| ATOM | 822 | CE | LYS | A | 103 | 22.439 | −19.760 | 23.457 | 1.00 | 31.01 | 6 | C |
| ATOM | 823 | NZ | LYS | A | 103 | 22.005 | −19.148 | 24.739 | 1.00 | 30.98 | 7 | N |
| ATOM | 824 | N | VAL | A | 104 | 26.655 | −17.986 | 17.568 | 1.00 | 21.14 | 7 | N |
| ATOM | 825 | CA | VAL | A | 104 | 26.902 | −17.009 | 16.508 | 1.00 | 22.00 | 6 | C |
| ATOM | 826 | C | VAL | A | 104 | 26.635 | −17.646 | 15.149 | 1.00 | 22.46 | 6 | C |
| ATOM | 827 | O | VAL | A | 104 | 27.267 | −18.636 | 14.774 | 1.00 | 24.42 | 8 | O |
| ATOM | 828 | CB | VAL | A | 104 | 28.342 | −16.472 | 16.516 | 1.00 | 22.01 | 6 | C |
| ATOM | 829 | CG1 | VAL | A | 104 | 28.578 | −15.530 | 15.339 | 1.00 | 21.75 | 6 | C |
| ATOM | 830 | CG2 | VAL | A | 104 | 28.596 | −15.724 | 17.817 | 1.00 | 21.75 | 6 | C |
| ATOM | 831 | N | GLU | A | 105 | 25.720 | −17.053 | 14.410 | 1.00 | 20.64 | 7 | N |
| ATOM | 832 | CA | GLU | A | 105 | 25.318 | −17.517 | 13.090 | 1.00 | 21.19 | 6 | C |
| ATOM | 833 | C | GLU | A | 105 | 25.817 | −16.500 | 12.068 | 1.00 | 20.81 | 6 | C |
| ATOM | 834 | O | GLU | A | 105 | 26.237 | −15.416 | 12.490 | 1.00 | 20.45 | 8 | O |
| ATOM | 835 | CB | GLU | A | 105 | 23.799 | −17.603 | 13.031 | 1.00 | 22.28 | 6 | C |
| ATOM | 836 | CG | GLU | A | 105 | 23.102 | −18.460 | 14.054 | 1.00 | 24.14 | 6 | C |
| ATOM | 837 | CD | GLU | A | 105 | 23.011 | −19.935 | 13.723 | 1.00 | 25.23 | 6 | C |
| ATOM | 838 | OE1 | GLU | A | 105 | 23.503 | −20.367 | 12.663 | 1.00 | 24.94 | 8 | O |
| ATOM | 839 | OE2 | GLU | A | 105 | 22.428 | −20.685 | 14.548 | 1.00 | 25.76 | 8 | O |
| ATOM | 840 | N | ARG | A | 106 | 25.790 | −16.822 | 10.778 | 1.00 | 20.46 | 7 | N |
| ATOM | 841 | CA | ARG | A | 106 | 26.282 | −15.846 | 9.815 | 1.00 | 20.88 | 6 | C |
| ATOM | 842 | C | ARG | A | 106 | 25.533 | −15.930 | 8.483 | 1.00 | 20.65 | 6 | C |
| ATOM | 843 | O | ARG | A | 106 | 25.104 | −17.008 | 8.103 | 1.00 | 18.99 | 8 | O |
| ATOM | 844 | CB | ARG | A | 106 | 27.780 | −16.036 | 9.572 | 1.00 | 23.75 | 6 | C |
| ATOM | 845 | CG | ARG | A | 106 | 28.220 | −17.442 | 9.249 | 1.00 | 28.27 | 6 | C |
| ATOM | 846 | CD | ARG | A | 106 | 29.625 | −17.572 | 8.744 | 1.00 | 32.56 | 6 | C |
| ATOM | 847 | NE | ARG | A | 106 | 30.758 | −17.238 | 9.536 | 1.00 | 36.69 | 7 | N |
| ATOM | 848 | CZ | ARG | A | 106 | 31.024 | −16.882 | 10.770 | 1.00 | 38.12 | 6 | C |
| ATOM | 849 | NH1 | ARG | A | 106 | 30.083 | −16.728 | 11.677 | 1.00 | 39.25 | 7 | N |
| ATOM | 850 | NH2 | ARG | A | 106 | 32.284 | −16.653 | 11.151 | 1.00 | 39.60 | 7 | N |
| ATOM | 851 | N | ILE | A | 107 | 25.431 | −14.789 | 7.822 | 1.00 | 19.08 | 7 | N |
| ATOM | 852 | CA | ILE | A | 107 | 24.821 | −14.735 | 6.492 | 1.00 | 19.20 | 6 | C |
| ATOM | 853 | C | ILE | A | 107 | 25.933 | −15.092 | 5.511 | 1.00 | 20.60 | 6 | C |
| ATOM | 854 | O | ILE | A | 107 | 27.068 | −14.684 | 5.761 | 1.00 | 20.44 | 8 | O |
| ATOM | 855 | CB | ILE | A | 107 | 24.314 | −13.323 | 6.164 | 1.00 | 17.34 | 6 | C |
| ATOM | 856 | CG1 | ILE | A | 107 | 23.277 | −12.864 | 7.172 | 1.00 | 17.26 | 6 | C |
| ATOM | 857 | CG2 | ILE | A | 107 | 23.792 | −13.246 | 4.720 | 1.00 | 17.31 | 6 | C |
| ATOM | 858 | CD1 | ILE | A | 107 | 21.982 | −13.637 | 7.234 | 1.00 | 18.86 | 6 | C |
| ATOM | 859 | N | GLU | A | 108 | 25.637 | −15.804 | 4.423 | 1.00 | 22.59 | 7 | N |
| ATOM | 860 | CA | GLU | A | 108 | 26.651 | −16.147 | 3.451 | 1.00 | 25.12 | 6 | C |
| ATOM | 861 | C | GLU | A | 108 | 26.250 | −15.757 | 2.022 | 1.00 | 24.79 | 6 | C |
| ATOM | 862 | O | GLU | A | 108 | 25.096 | −15.437 | 1.777 | 1.00 | 25.02 | 8 | O |
| ATOM | 863 | CB | GLU | A | 108 | 26.855 | −17.647 | 3.344 | 1.00 | 28.51 | 6 | C |
| ATOM | 864 | CG | GLU | A | 108 | 27.198 | −18.470 | 4.549 | 1.00 | 32.86 | 6 | C |
| ATOM | 865 | CD | GLU | A | 108 | 27.174 | −19.938 | 4.126 | 1.00 | 35.94 | 6 | C |
| ATOM | 866 | OE1 | GLU | A | 108 | 26.667 | −20.241 | 3.031 | 1.00 | 37.52 | 8 | O |
| ATOM | 867 | OE2 | GLU | A | 108 | 27.681 | −20.735 | 4.935 | 1.00 | 38.02 | 8 | O |
| ATOM | 868 | N | ALA | A | 109 | 27.206 | −15.832 | 1.099 | 1.00 | 24.76 | 7 | N |
| ATOM | 869 | CA | ALA | A | 109 | 26.860 | −15.518 | −0.296 | 1.00 | 25.18 | 6 | C |
| ATOM | 870 | C | ALA | A | 109 | 25.866 | −16.570 | −0.768 | 1.00 | 24.97 | 6 | C |
| ATOM | 871 | O | ALA | A | 109 | 25.843 | −17.699 | −0.281 | 1.00 | 23.19 | 8 | O |
| ATOM | 872 | CB | ALA | A | 109 | 28.099 | −15.557 | −1.178 | 1.00 | 26.77 | 6 | C |
| ATOM | 873 | N | CPR | A | 110 | 24.995 | −16.198 | −1.702 | 1.00 | 24.48 | 7 | N |
| ATOM | 874 | CA | CPR | A | 110 | 24.957 | −14.885 | −2.318 | 1.00 | 24.48 | 6 | C |
| ATOM | 875 | C | CPR | A | 110 | 24.047 | −13.865 | −1.654 | 1.00 | 24.12 | 6 | C |
| ATOM | 876 | O | CPR | A | 110 | 23.522 | −12.933 | −2.286 | 1.00 | 23.83 | 8 | O |
| ATOM | 877 | CB | CPR | A | 110 | 24.425 | −15.250 | −3.714 | 1.00 | 24.65 | 6 | C |
| ATOM | 878 | CG | CPR | A | 110 | 23.443 | −16.345 | −3.448 | 1.00 | 24.70 | 6 | C |
| ATOM | 879 | CD | CPR | A | 110 | 23.976 | −17.115 | −2.271 | 1.00 | 24.77 | 6 | C |
| ATOM | 880 | N | GLY | A | 111 | 23.857 | −13.986 | −0.352 | 1.00 | 22.42 | 7 | N |
| ATOM | 881 | CA | GLY | A | 111 | 22.996 | −13.089 | 0.416 | 1.00 | 21.93 | 6 | C |
| ATOM | 882 | C | GLY | A | 111 | 23.797 | −11.865 | 0.861 | 1.00 | 21.71 | 6 | C |
| ATOM | 883 | O | GLY | A | 111 | 25.008 | −11.883 | 1.016 | 1.00 | 21.47 | 8 | O |
| ATOM | 884 | N | THR | A | 112 | 23.075 | −10.786 | 1.058 | 1.00 | 21.73 | 7 | N |
| ATOM | 885 | CA | THR | A | 112 | 23.649 | −9.490 | 1.480 | 1.00 | 20.56 | 6 | C |
| ATOM | 886 | C | THR | A | 112 | 22.691 | −8.974 | 2.532 | 1.00 | 20.10 | 6 | C |
| ATOM | 887 | O | THR | A | 112 | 21.458 | −8.981 | 2.324 | 1.00 | 17.76 | 8 | O |
| ATOM | 888 | CB | THR | A | 112 | 23.733 | −8.793 | 0.105 | 1.00 | 23.31 | 6 | C |
| ATOM | 889 | OG1 | THR | A | 112 | 25.076 | −8.697 | −0.421 | 1.00 | 23.48 | 8 | O |
| ATOM | 890 | CG2 | THR | A | 112 | 22.988 | −7.501 | 0.043 | 1.00 | 21.75 | 6 | C |
| ATOM | 891 | N | VAL | A | 113 | 23.134 | −8.610 | 3.748 | 1.00 | 17.43 | 7 | N |
| ATOM | 892 | CA | VAL | A | 113 | 22.222 | −8.105 | 4.769 | 1.00 | 17.82 | 6 | C |
| ATOM | 893 | C | VAL | A | 113 | 22.919 | −6.983 | 5.555 | 1.00 | 17.33 | 6 | C |
| ATOM | 894 | O | VAL | A | 113 | 24.080 | −7.166 | 5.880 | 1.00 | 16.70 | 8 | O |
| ATOM | 895 | CB | VAL | A | 113 | 21.757 | −9.093 | 5.851 | 1.00 | 17.34 | 6 | C |
| ATOM | 896 | CG1 | VAL | A | 113 | 20.962 | −8.438 | 6.990 | 1.00 | 17.11 | 6 | C |
| ATOM | 897 | CG2 | VAL | A | 113 | 20.848 | −10.187 | 5.303 | 1.00 | 17.73 | 6 | C |
| ATOM | 898 | N | GLU | A | 114 | 22.218 | −5.907 | 5.853 | 1.00 | 17.12 | 7 | N |
| ATOM | 899 | CA | GLU | A | 114 | 22.736 | −4.838 | 6.705 | 1.00 | 17.08 | 6 | C |
| ATOM | 900 | C | GLU | A | 114 | 21.636 | −4.708 | 7.768 | 1.00 | 16.97 | 6 | C |
| ATOM | 901 | O | GLU | A | 114 | 20.498 | −4.427 | 7.395 | 1.00 | 15.88 | 8 | O |
| ATOM | 902 | CB | GLU | A | 114 | 23.080 | −3.517 | 6.038 | 1.00 | 16.82 | 6 | C |
| ATOM | 903 | CG | GLU | A | 114 | 24.358 | −3.636 | 5.228 | 1.00 | 16.48 | 6 | C |
| ATOM | 904 | CD | GLU | A | 114 | 24.735 | −2.441 | 4.384 | 1.00 | 17.32 | 6 | C |
| ATOM | 905 | OE1 | GLU | A | 114 | 24.217 | −1.334 | 4.621 | 1.00 | 17.04 | 8 | O |
| ATOM | 906 | OE2 | GLU | A | 114 | 25.572 | −2.639 | 3.476 | 1.00 | 18.05 | 8 | O |
| ATOM | 907 | N | ALA | A | 115 | 21.976 | −4.910 | 9.052 | 1.00 | 16.45 | 7 | N |
| ATOM | 908 | CA | ALA | A | 115 | 21.001 | −4.904 | 10.126 | 1.00 | 15.18 | 6 | C |
| ATOM | 909 | C | ALA | A | 115 | 20.195 | −3.657 | 10.379 | 1.00 | 15.13 | 6 | C |
| ATOM | 910 | O | ALA | A | 115 | 19.192 | −3.705 | 11.093 | 1.00 | 14.40 | 8 | O |
| ATOM | 911 | CB | ALA | A | 115 | 21.584 | −5.425 | 11.457 | 1.00 | 15.94 | 6 | C |
| ATOM | 912 | N | GLY | A | 116 | 20.513 | −2.532 | 9.750 | 1.00 | 15.78 | 7 | N |
| ATOM | 913 | CA | GLY | A | 116 | 19.739 | −1.300 | 9.858 | 1.00 | 16.57 | 6 | C |
| ATOM | 914 | C | GLY | A | 116 | 18.400 | −1.514 | 9.137 | 1.00 | 16.27 | 6 | C |
| ATOM | 915 | O | GLY | A | 116 | 17.425 | −0.790 | 9.322 | 1.00 | 15.64 | 8 | O |
| ATOM | 916 | N | ASP | A | 117 | 18.322 | −2.557 | 8.301 | 1.00 | 16.72 | 7 | N |
| ATOM | 917 | CA | ASP | A | 117 | 17.138 | −2.955 | 7.572 | 1.00 | 17.01 | 6 | C |
| ATOM | 918 | C | ASP | A | 117 | 16.229 | −3.841 | 8.430 | 1.00 | 17.80 | 6 | C |
| ATOM | 919 | O | ASP | A | 117 | 15.163 | −4.258 | 7.989 | 1.00 | 19.09 | 8 | O |
| ATOM | 920 | CB | ASP | A | 117 | 17.523 | −3.737 | 6.307 | 1.00 | 16.92 | 6 | C |
| ATOM | 921 | CG | ASP | A | 117 | 17.733 | −2.859 | 5.089 | 1.00 | 16.64 | 6 | C |
| ATOM | 922 | OD1 | ASP | A | 117 | 16.860 | −1.992 | 4.855 | 1.00 | 16.06 | 8 | O |
| ATOM | 923 | OD2 | ASP | A | 117 | 18.751 | −3.034 | 4.375 | 1.00 | 16.26 | 8 | O |
| ATOM | 924 | N | ILE | A | 118 | 16.663 | −4.140 | 9.660 | 1.00 | 17.25 | 7 | N |
| ATOM | 925 | CA | ILE | A | 118 | 15.904 | −5.005 | 10.559 | 1.00 | 16.61 | 6 | C |
| ATOM | 926 | C | ILE | A | 118 | 15.345 | −4.305 | 11.776 | 1.00 | 17.42 | 6 | C |
| ATOM | 927 | O | ILE | A | 118 | 16.031 | −3.687 | 12.606 | 1.00 | 16.87 | 8 | O |
| ATOM | 928 | CB | ILE | A | 118 | 16.762 | −6.226 | 10.972 | 1.00 | 16.59 | 6 | C |
| ATOM | 929 | CG1 | ILE | A | 118 | 17.276 | −6.961 | 9.731 | 1.00 | 16.43 | 6 | C |
| ATOM | 930 | CG2 | ILE | A | 118 | 15.939 | −7.203 | 11.833 | 1.00 | 16.16 | 6 | C |
| ATOM | 931 | CD1 | ILE | A | 118 | 18.284 | −8.065 | 9.996 | 1.00 | 17.93 | 6 | C |
| ATOM | 932 | N | MET | A | 119 | 14.017 | −4.446 | 11.956 | 1.00 | 17.12 | 7 | N |
| ATOM | 933 | CA | MET | A | 119 | 13.310 | −3.881 | 13.086 | 1.00 | 17.19 | 6 | C |
| ATOM | 934 | C | MET | A | 119 | 12.711 | −4.964 | 13.991 | 1.00 | 17.81 | 6 | C |
| ATOM | 935 | O | MET | A | 119 | 11.854 | −5.722 | 13.551 | 1.00 | 17.29 | 8 | O |
| ATOM | 936 | CB | MET | A | 119 | 12.194 | −2.941 | 12.624 | 1.00 | 17.53 | 6 | C |
| ATOM | 937 | CG | MET | A | 119 | 11.537 | −2.207 | 13.788 | 1.00 | 19.81 | 6 | C |
| ATOM | 938 | SD | MET | A | 119 | 10.283 | −1.020 | 13.276 | 1.00 | 18.46 | 16 | S |
| ATOM | 939 | CE | MET | A | 119 | 9.142 | −2.101 | 12.395 | 1.00 | 21.15 | 6 | C |
| ATOM | 940 | N | MET | A | 120 | 13.168 | −5.064 | 15.235 | 1.00 | 15.58 | 7 | N |
| ATOM | 941 | CA | MET | A | 120 | 12.710 | −6.059 | 16.191 | 1.00 | 15.75 | 6 | C |
| ATOM | 942 | C | MET | A | 120 | 11.492 | −5.605 | 16.973 | 1.00 | 15.97 | 6 | C |
| ATOM | 943 | O | MET | A | 120 | 11.515 | −4.571 | 17.660 | 1.00 | 16.43 | 8 | O |
| ATOM | 944 | CB | MET | A | 120 | 13.834 | −6.355 | 17.205 | 1.00 | 16.59 | 6 | C |
| ATOM | 945 | CG | MET | A | 120 | 13.470 | −7.291 | 18.336 | 1.00 | 16.54 | 6 | C |
| ATOM | 946 | SD | MET | A | 120 | 14.890 | −7.715 | 19.407 | 1.00 | 17.57 | 16 | S |
| ATOM | 947 | CE | MET | A | 120 | 14.917 | −6.219 | 20.413 | 1.00 | 18.74 | 6 | C |
| ATOM | 948 | N | VAL | A | 121 | 10.420 | −6.377 | 16.877 | 1.00 | 17.00 | 7 | N |
| ATOM | 949 | CA | VAL | A | 121 | 9.174 | −6.081 | 17.602 | 1.00 | 17.56 | 6 | C |
| ATOM | 950 | C | VAL | A | 121 | 8.827 | −7.363 | 18.360 | 1.00 | 19.01 | 6 | C |
| ATOM | 951 | O | VAL | A | 121 | 8.249 | −8.249 | 17.731 | 1.00 | 17.79 | 8 | O |
| ATOM | 952 | CB | VAL | A | 121 | 7.984 | −5.707 | 16.710 | 1.00 | 18.36 | 6 | C |
| ATOM | 953 | CG1 | VAL | A | 121 | 6.758 | −5.327 | 17.549 | 1.00 | 16.86 | 6 | C |
| ATOM | 954 | CG2 | VAL | A | 121 | 8.347 | −4.562 | 15.778 | 1.00 | 17.67 | 6 | C |
| ATOM | 955 | N | GLY | A | 122 | 9.213 | −7.456 | 19.632 | 1.00 | 19.78 | 7 | N |
| ATOM | 956 | CA | GLY | A | 122 | 8.928 | −8.721 | 20.341 | 1.00 | 22.19 | 6 | C |
| ATOM | 957 | C | GLY | A | 122 | 9.821 | −9.791 | 19.712 | 1.00 | 22.59 | 6 | C |
| ATOM | 958 | O | GLY | A | 122 | 11.020 | −9.550 | 19.489 | 1.00 | 21.74 | 8 | O |
| ATOM | 959 | N | ASP | A | 123 | 9.263 | −10.955 | 19.383 | 1.00 | 22.60 | 7 | N |
| ATOM | 960 | CA | ASP | A | 123 | 10.091 | −11.996 | 18.754 | 1.00 | 23.03 | 6 | C |
| ATOM | 961 | C | ASP | A | 123 | 9.973 | −11.965 | 17.230 | 1.00 | 22.78 | 6 | C |
| ATOM | 962 | O | ASP | A | 123 | 10.463 | −12.863 | 16.527 | 1.00 | 23.84 | 8 | O |
| ATOM | 963 | CB | ASP | A | 123 | 9.719 | −13.361 | 19.325 | 1.00 | 24.99 | 6 | C |
| ATOM | 964 | CG | ASP | A | 123 | 9.970 | −13.510 | 20.809 | 1.00 | 26.48 | 6 | C |
| ATOM | 965 | OD1 | ASP | A | 123 | 10.944 | −12.932 | 21.341 | 1.00 | 26.50 | 8 | O |
| ATOM | 966 | OD2 | ASP | A | 123 | 9.179 | −14.222 | 21.473 | 1.00 | 27.69 | 8 | O |
| ATOM | 967 | N | HIS | A | 124 | 9.342 | −10.933 | 16.670 | 1.00 | 22.44 | 7 | N |
| ATOM | 968 | CA | HIS | A | 124 | 9.184 | −10.808 | 15.225 | 1.00 | 20.66 | 6 | C |
| ATOM | 969 | C | HIS | A | 124 | 10.207 | −9.801 | 14.696 | 1.00 | 20.78 | 6 | C |
| ATOM | 970 | O | HIS | A | 124 | 10.418 | −8.767 | 15.340 | 1.00 | 20.50 | 8 | O |
| ATOM | 971 | CB | HIS | A | 124 | 7.785 | −10.357 | 14.790 | 1.00 | 20.71 | 6 | C |
| ATOM | 972 | CG | HIS | A | 124 | 7.598 | −10.514 | 13.306 | 1.00 | 20.90 | 6 | C |
| ATOM | 973 | ND1 | HIS | A | 124 | 7.243 | −11.730 | 12.751 | 1.00 | 21.98 | 7 | N |
| ATOM | 974 | CD2 | HIS | A | 124 | 7.718 | −9.649 | 12.273 | 1.00 | 20.62 | 6 | C |
| ATOM | 975 | CE1 | HIS | A | 124 | 7.147 | −11.599 | 11.432 | 1.00 | 21.32 | 6 | C |
| ATOM | 976 | NE2 | HIS | A | 124 | 7.432 | −10.342 | 11.120 | 1.00 | 20.94 | 7 | N |
| ATOM | 977 | N | PHE | A | 125 | 10.823 | −10.129 | 13.566 | 1.00 | 18.77 | 7 | N |
| ATOM | 978 | CA | PHE | A | 125 | 11.835 | −9.259 | 12.964 | 1.00 | 18.58 | 6 | C |
| ATOM | 979 | C | PHE | A | 125 | 11.421 | −8.813 | 11.568 | 1.00 | 18.44 | 6 | C |
| ATOM | 980 | O | PHE | A | 125 | 11.288 | −9.638 | 10.663 | 1.00 | 17.19 | 8 | O |
| ATOM | 981 | CB | PHE | A | 125 | 13.204 | −9.959 | 12.921 | 1.00 | 18.85 | 6 | C |
| ATOM | 982 | CG | PHE | A | 125 | 13.817 | −10.154 | 14.282 | 1.00 | 19.01 | 6 | C |
| ATOM | 983 | CD1 | PHE | A | 125 | 13.413 | −11.229 | 15.068 | 1.00 | 20.24 | 6 | C |
| ATOM | 984 | CD2 | PHE | A | 125 | 14.765 | −9.287 | 14.783 | 1.00 | 18.97 | 6 | C |
| ATOM | 985 | CE1 | PHE | A | 125 | 13.971 | −11.409 | 16.330 | 1.00 | 19.75 | 6 | C |
| ATOM | 986 | CE2 | PHE | A | 125 | 15.323 | −9.454 | 16.032 | 1.00 | 19.58 | 6 | C |
| ATOM | 987 | CZ | PHE | A | 125 | 14.910 | −10.531 | 16.805 | 1.00 | 20.22 | 6 | C |
| ATOM | 988 | N | TYR | A | 126 | 11.186 | −7.508 | 11.386 | 1.00 | 18.48 | 7 | N |
| ATOM | 989 | CA | TYR | A | 126 | 10.789 | −6.992 | 10.068 | 1.00 | 17.77 | 6 | C |
| ATOM | 990 | C | TYR | A | 126 | 12.069 | −6.643 | 9.315 | 1.00 | 17.48 | 6 | C |
| ATOM | 991 | O | TYR | A | 126 | 12.912 | −5.919 | 9.853 | 1.00 | 17.76 | 8 | O |
| ATOM | 992 | CB | TYR | A | 126 | 9.935 | −5.729 | 10.135 | 1.00 | 17.74 | 6 | C |
| ATOM | 993 | CG | TYR | A | 126 | 8.579 | −5.972 | 10.757 | 1.00 | 17.43 | 6 | C |
| ATOM | 994 | CD1 | TYR | A | 126 | 8.398 | −5.795 | 12.111 | 1.00 | 18.79 | 6 | C |
| ATOM | 995 | CD2 | TYR | A | 126 | 7.498 | −6.374 | 9.989 | 1.00 | 18.61 | 6 | C |
| ATOM | 996 | CE1 | TYR | A | 126 | 7.166 | −6.010 | 12.713 | 1.00 | 18.91 | 6 | C |
| ATOM | 997 | CE2 | TYR | A | 126 | 6.256 | −6.595 | 10.569 | 1.00 | 19.64 | 6 | C |
| ATOM | 998 | CZ | TYR | A | 126 | 6.108 | −6.412 | 11.927 | 1.00 | 20.01 | 6 | C |
| ATOM | 999 | OH | TYR | A | 126 | 4.876 | −6.621 | 12.534 | 1.00 | 19.92 | 8 | O |
| ATOM | 1000 | N | ILE | A | 127 | 12.216 | −7.179 | 8.116 | 1.00 | 16.69 | 7 | N |
| ATOM | 1001 | CA | LE | A | 127 | 13.408 | −6.978 | 7.318 | 1.00 | 16.11 | 6 | C |
| ATOM | 1002 | C | ILE | A | 127 | 13.140 | −6.275 | 5.994 | 1.00 | 16.73 | 6 | C |
| ATOM | 1003 | O | ILE | A | 127 | 12.428 | −6.831 | 5.153 | 1.00 | 15.46 | 8 | O |
| ATOM | 1004 | CB | ILE | A | 127 | 14.041 | −8.361 | 7.004 | 1.00 | 16.81 | 6 | C |
| ATOM | 1005 | CG1 | ILE | A | 127 | 14.370 | −9.160 | 8.270 | 1.00 | 17.54 | 6 | C |
| ATOM | 1006 | CG2 | ILE | A | 127 | 15.302 | −8.146 | 6.170 | 1.00 | 15.38 | 6 | C |
| ATOM | 1007 | CD1 | ILE | A | 127 | 14.843 | −10.572 | 7.962 | 1.00 | 18.57 | 6 | C |
| ATOM | 1008 | N | GLY | A | 128 | 13.716 | −5.086 | 5.803 | 1.00 | 16.59 | 7 | N |
| ATOM | 1009 | CA | GLY | A | 128 | 13.506 | −4.374 | 4.545 | 1.00 | 17.40 | 6 | C |
| ATOM | 1010 | C | GLY | A | 128 | 14.355 | −4.895 | 3.390 | 1.00 | 19.28 | 6 | C |
| ATOM | 1011 | O | GLY | A | 128 | 15.517 | −5.313 | 3.512 | 1.00 | 18.81 | 8 | O |
| ATOM | 1012 | N | GLU | A | 129 | 13.740 | −4.901 | 2.200 | 1.00 | 18.92 | 7 | N |
| ATOM | 1013 | CA | GLU | A | 129 | 14.410 | −5.317 | 0.974 | 1.00 | 18.60 | 6 | C |
| ATOM | 1014 | C | GLU | A | 129 | 14.956 | −4.005 | 0.399 | 1.00 | 18.73 | 6 | C |
| ATOM | 1015 | O | GLU | A | 129 | 14.176 | −3.227 | −0.150 | 1.00 | 18.40 | 8 | O |
| ATOM | 1016 | CB | GLU | A | 129 | 13.520 | −6.031 | −0.030 | 1.00 | 18.87 | 6 | C |
| ATOM | 1017 | CG | GLU | A | 129 | 14.251 | −6.404 | −1.330 | 1.00 | 19.11 | 6 | C |
| ATOM | 1018 | CD | GLU | A | 129 | 13.317 | −7.047 | −2.344 | 1.00 | 21.11 | 6 | C |
| ATOM | 1019 | OE1 | GLU | A | 129 | 12.125 | −6.689 | −2.337 | 1.00 | 21.19 | 8 | O |
| ATOM | 1020 | OE2 | GLU | A | 129 | 13.776 | −7.906 | −3.127 | 1.00 | 21.49 | 8 | O |
| ATOM | 1021 | N | SER | A | 130 | 16.240 | −3.742 | 0.551 | 1.00 | 17.84 | 7 | N |
| ATOM | 1022 | CA | SER | A | 130 | 16.789 | −2.474 | 0.065 | 1.00 | 17.66 | 6 | C |
| ATOM | 1023 | C | SER | A | 130 | 17.951 | −2.662 | −0.877 | 1.00 | 17.82 | 6 | C |
| ATOM | 1024 | O | SER | A | 130 | 18.185 | −3.763 | −1.376 | 1.00 | 18.37 | 8 | O |
| ATOM | 1025 | CB | SER | A | 130 | 17.252 | −1.705 | 1.331 | 1.00 | 18.49 | 6 | C |
| ATOM | 1026 | OG | SER | A | 130 | 18.354 | −2.416 | 1.889 | 1.00 | 17.00 | 8 | O |
| ATOM | 1027 | N | ALA | A | 131 | 18.718 | −1.591 | −1.105 | 1.00 | 18.18 | 7 | N |
| ATOM | 1028 | CA | ALA | A | 131 | 19.895 | −1.687 | −1.966 | 1.00 | 19.72 | 6 | C |
| ATOM | 1029 | C | ALA | A | 131 | 21.026 | −2.401 | −1.227 | 1.00 | 19.78 | 6 | C |
| ATOM | 1030 | O | ALA | A | 131 | 21.985 | −2.873 | −1.852 | 1.00 | 19.62 | 8 | O |
| ATOM | 1031 | CB | ALA | A | 131 | 20.365 | −0.299 | −2.379 | 1.00 | 20.76 | 6 | C |
| ATOM | 1032 | N | ARG | A | 132 | 20.920 | −2.464 | 0.099 | 1.00 | 19.88 | 7 | N |
| ATOM | 1033 | CA | ARG | A | 132 | 21.919 | −3.100 | 0.945 | 1.00 | 19.48 | 6 | C |
| ATOM | 1034 | C | ARG | A | 132 | 21.518 | −4.486 | 1.428 | 1.00 | 19.66 | 6 | C |
| ATOM | 1035 | O | ARG | A | 132 | 22.389 | −5.305 | 1.728 | 1.00 | 22.44 | 8 | O |
| ATOM | 1036 | CB | ARG | A | 132 | 22.162 | −2.204 | 2.166 | 1.00 | 18.99 | 6 | C |
| ATOM | 1037 | CG | ARG | A | 132 | 22.607 | −0.789 | 1.816 | 1.00 | 19.20 | 6 | C |
| ATOM | 1038 | CD | ARG | A | 132 | 24.025 | −0.808 | 1.252 | 1.00 | 19.21 | 6 | C |
| ATOM | 1039 | NE | ARG | A | 132 | 24.449 | 0.539 | 0.852 | 1.00 | 19.49 | 7 | N |
| ATOM | 1040 | CZ | ARG | A | 132 | 24.902 | 1.409 | 1.748 | 1.00 | 20.42 | 6 | C |
| ATOM | 1041 | NH1 | ARG | A | 132 | 24.992 | 1.066 | 3.033 | 1.00 | 20.41 | 7 | N |
| ATOM | 1042 | NH2 | ARG | A | 132 | 25.305 | 2.641 | 1.440 | 1.00 | 20.39 | 7 | N |
| ATOM | 1043 | N | THR | A | 133 | 20.243 | −4.789 | 1.553 | 1.00 | 17.40 | 7 | N |
| ATOM | 1044 | CA | THR | A | 133 | 19.748 | −6.080 | 2.001 | 1.00 | 17.55 | 6 | C |
| ATOM | 1045 | C | THR | A | 133 | 18.910 | −6.718 | 0.890 | 1.00 | 17.95 | 6 | C |
| ATOM | 1046 | O | THR | A | 133 | 17.809 | −6.228 | 0.675 | 1.00 | 16.30 | 8 | O |
| ATOM | 1047 | CB | THR | A | 133 | 18.889 | −5.928 | 3.264 | 1.00 | 17.41 | 6 | C |
| ATOM | 1048 | OG1 | THR | A | 133 | 19.752 | −5.471 | 4.322 | 1.00 | 16.82 | 8 | O |
| ATOM | 1049 | CG2 | THR | A | 133 | 18.258 | −7.232 | 3.735 | 1.00 | 19.29 | 6 | C |
| ATOM | 1050 | N | ASN | A | 134 | 19.427 | −7.776 | 0.284 | 1.00 | 17.65 | 7 | N |
| ATOM | 1051 | CA | ASN | A | 134 | 18.680 | −8.426 | −0.803 | 1.00 | 17.36 | 6 | C |
| ATOM | 1052 | C | ASN | A | 134 | 17.788 | −9.543 | −0.288 | 1.00 | 17.32 | 6 | C |
| ATOM | 1053 | O | ASN | A | 134 | 17.869 | −9.994 | 0.851 | 1.00 | 16.56 | 8 | O |
| ATOM | 1054 | CB | ASN | A | 134 | 19.630 | −8.857 | −1.900 | 1.00 | 17.19 | 6 | C |
| ATOM | 1055 | CG | ASN | A | 134 | 20.589 | −9.972 | −1.571 | 1.00 | 17.42 | 6 | C |
| ATOM | 1056 | OD1 | ASN | A | 134 | 20.444 | −10.674 | −0.570 | 1.00 | 17.45 | 8 | O |
| ATOM | 1057 | ND2 | ASN | A | 134 | 21.590 | −10.142 | −2.434 | 1.00 | 17.63 | 7 | N |
| ATOM | 1058 | N | ALA | A | 135 | 16.858 | −10.002 | −1.131 | 1.00 | 17.60 | 7 | N |
| ATOM | 1059 | CA | ALA | A | 135 | 15.916 | −11.056 | −0.776 | 1.00 | 18.44 | 6 | C |
| ATOM | 1060 | C | ALA | A | 135 | 16.555 | −12.362 | −0.335 | 1.00 | 18.49 | 6 | C |
| ATOM | 1061 | O | ALA | A | 135 | 16.001 | −13.082 | 0.507 | 1.00 | 18.20 | 8 | O |
| ATOM | 1062 | CB | ALA | A | 135 | 15.013 | −11.305 | −1.991 | 1.00 | 19.02 | 6 | C |
| ATOM | 1063 | N | GLU | A | 136 | 17.711 | −12.707 | −0.888 | 1.00 | 18.70 | 7 | N |
| ATOM | 1064 | CA | GLU | A | 136 | 18.405 | −13.942 | −0.507 | 1.00 | 19.08 | 6 | C |
| ATOM | 1065 | C | GLU | A | 136 | 18.928 | −13.779 | 0.927 | 1.00 | 18.39 | 6 | C |
| ATOM | 1066 | O | GLU | A | 136 | 18.832 | −14.694 | 1.726 | 1.00 | 18.42 | 8 | O |
| ATOM | 1067 | CB | GLU | A | 136 | 19.552 | −14.291 | −1.432 | 1.00 | 19.03 | 6 | C |
| ATOM | 1068 | CG | GLU | A | 136 | 20.480 | −15.402 | −0.996 | 1.00 | 20.57 | 6 | C |
| ATOM | 1069 | CD | GLU | A | 136 | 19.848 | −16.746 | −0.730 | 1.00 | 21.96 | 6 | C |
| ATOM | 1070 | OE1 | GLU | A | 136 | 18.726 | −17.004 | −1.231 | 1.00 | 22.96 | 8 | O |
| ATOM | 1071 | OE2 | GLU | A | 136 | 20.460 | −17.575 | −0.013 | 1.00 | 21.43 | 8 | O |
| ATOM | 1072 | N | GLY | A | 137 | 19.449 | −12.595 | 1.228 | 1.00 | 18.02 | 7 | N |
| ATOM | 1073 | CA | GLY | A | 137 | 19.971 | −12.296 | 2.565 | 1.00 | 17.74 | 6 | C |
| ATOM | 1074 | C | GLY | A | 137 | 18.835 | −12.329 | 3.577 | 1.00 | 17.14 | 6 | C |
| ATOM | 1075 | O | GLY | A | 137 | 18.955 | −12.871 | 4.678 | 1.00 | 17.23 | 8 | O |
| ATOM | 1076 | N | ALA | A | 138 | 17.714 | −11.715 | 3.223 | 1.00 | 16.38 | 7 | N |
| ATOM | 1077 | CA | ALA | A | 138 | 16.538 | −11.709 | 4.082 | 1.00 | 16.52 | 6 | C |
| ATOM | 1078 | C | ALA | A | 138 | 16.106 | −13.145 | 4.347 | 1.00 | 17.36 | 6 | C |
| ATOM | 1079 | O | ALA | A | 138 | 15.761 | −13.520 | 5.462 | 1.00 | 17.35 | 8 | O |
| ATOM | 1080 | CB | ALA | A | 138 | 15.379 | −10.963 | 3.422 | 1.00 | 17.23 | 6 | C |
| ATOM | 1081 | N | ARG | A | 139 | 16.077 | −13.973 | 3.299 | 1.00 | 18.51 | 7 | N |
| ATOM | 1082 | CA | ARG | A | 139 | 15.693 | −15.375 | 3.397 | 1.00 | 19.63 | 6 | C |
| ATOM | 1083 | C | ARG | A | 139 | 16.580 | −16.126 | 4.387 | 1.00 | 20.20 | 6 | C |
| ATOM | 1084 | O | ARG | A | 139 | 16.094 | −16.837 | 5.254 | 1.00 | 19.75 | 8 | O |
| ATOM | 1085 | CB | ARG | A | 139 | 15.824 | −16.045 | 2.023 | 1.00 | 22.27 | 6 | C |
| ATOM | 1086 | CG | ARG | A | 139 | 15.388 | −17.507 | 2.010 | 1.00 | 24.93 | 6 | C |
| ATOM | 1087 | CD | ARG | A | 139 | 15.868 | −18.202 | 0.746 | 1.00 | 26.99 | 6 | C |
| ATOM | 1088 | NE | ARG | A | 139 | 17.285 | −18.520 | 0.760 | 1.00 | 29.07 | 7 | N |
| ATOM | 1089 | CZ | ARG | A | 139 | 17.862 | −19.494 | 1.455 | 1.00 | 30.03 | 6 | C |
| ATOM | 1090 | NH1 | ARG | A | 139 | 17.108 | −20.274 | 2.230 | 1.00 | 29.98 | 7 | N |
| ATOM | 1091 | NH2 | ARG | A | 139 | 19.175 | −19.692 | 1.382 | 1.00 | 29.77 | 7 | N |
| ATOM | 1092 | N | GLN | A | 140 | 17.897 | −15.962 | 4.230 | 1.00 | 19.21 | 7 | N |
| ATOM | 1093 | CA | GLN | A | 140 | 18.846 | −16.621 | 5.119 | 1.00 | 18.71 | 6 | C |
| ATOM | 1094 | C | GLN | A | 140 | 18.610 | −16.188 | 6.557 | 1.00 | 18.45 | 6 | C |
| ATOM | 1095 | O | GLN | A | 140 | 18.615 | −17.028 | 7.446 | 1.00 | 17.58 | 8 | O |
| ATOM | 1096 | CB | GLN | A | 140 | 20.271 | −16.310 | 4.665 | 1.00 | 18.99 | 6 | C |
| ATOM | 1097 | CG | GLN | A | 140 | 20.725 | −17.195 | 3.510 | 1.00 | 20.49 | 6 | C |
| ATOM | 1098 | CD | GLN | A | 140 | 22.171 | −16.865 | 3.174 | 1.00 | 20.82 | 6 | C |
| ATOM | 1099 | OE1 | GLN | A | 140 | 22.993 | −16.769 | 4.084 | 1.00 | 21.16 | 8 | O |
| ATOM | 1100 | NE2 | GLN | A | 140 | 22.449 | −16.694 | 1.889 | 1.00 | 21.04 | 7 | N |
| ATOM | 1101 | N | MET | A | 141 | 18.402 | −14.889 | 6.787 | 1.00 | 18.26 | 7 | N |
| ATOM | 1102 | CA | MET | A | 141 | 18.151 | −14.372 | 8.127 | 1.00 | 17.13 | 6 | C |
| ATOM | 1103 | C | MET | A | 141 | 16.852 | −14.913 | 8.701 | 1.00 | 16.54 | 6 | C |
| ATOM | 1104 | O | MET | A | 141 | 16.758 | −15.321 | 9.849 | 1.00 | 15.43 | 8 | O |
| ATOM | 1105 | CB | MET | A | 141 | 18.133 | −12.837 | 8.106 | 1.00 | 17.64 | 6 | C |
| ATOM | 1106 | CG | MET | A | 141 | 17.909 | −12.225 | 9.487 | 1.00 | 18.24 | 6 | C |
| ATOM | 1107 | SD | MET | A | 141 | 19.327 | −12.468 | 10.592 | 1.00 | 17.25 | 16 | S |
| ATOM | 1108 | CE | MET | A | 141 | 20.480 | −11.262 | 9.939 | 1.00 | 17.97 | 6 | C |
| ATOM | 1109 | N | ILE | A | 142 | 15.774 | −14.932 | 7.886 | 1.00 | 15.84 | 7 | N |
| ATOM | 1110 | CA | ILE | A | 142 | 14.511 | −15.465 | 8.438 | 1.00 | 17.22 | 6 | C |
| ATOM | 1111 | C | ILE | A | 142 | 14.678 | −26.924 | 8.841 | 1.00 | 17.33 | 6 | C |
| ATOM | 1112 | O | ILE | A | 142 | 14.156 | −17.341 | 9.883 | 1.00 | 18.42 | 8 | O |
| ATOM | 1113 | CB | ILE | A | 142 | 13.337 | −15.274 | 7.452 | 1.00 | 18.00 | 6 | C |
| ATOM | 1114 | CG1 | ILE | A | 142 | 13.000 | −13.767 | 7.422 | 1.00 | 19.18 | 6 | C |
| ATOM | 1115 | CG2 | ILE | A | 142 | 12.122 | −16.071 | 7.868 | 1.00 | 16.81 | 6 | C |
| ATOM | 1116 | CD1 | ILE | A | 142 | 12.176 | −13.316 | 6.237 | 1.00 | 17.82 | 6 | C |
| ATOM | 1117 | N | ALA | A | 143 | 15.406 | −17.722 | 8.060 | 1.00 | 18.10 | 7 | N |
| ATOM | 1118 | CA | ALA | A | 143 | 15.611 | −19.139 | 8.361 | 1.00 | 17.33 | 6 | C |
| ATOM | 1119 | C | ALA | A | 143 | 16.365 | −19.324 | 9.677 | 1.00 | 17.51 | 6 | C |
| ATOM | 1120 | O | ALA | A | 143 | 16.020 | −20.189 | 10.479 | 1.00 | 17.31 | 8 | O |
| ATOM | 1121 | CB | ALA | A | 143 | 16.309 | −19.842 | 7.209 | 1.00 | 16.91 | 6 | C |
| ATOM | 1122 | N | ILE | A | 144 | 17.378 | −18.497 | 9.914 | 1.00 | 17.89 | 7 | N |
| ATOM | 1123 | CA | ILE | A | 144 | 18.154 | −18.577 | 11.156 | 1.00 | 18.83 | 6 | C |
| ATOM | 1124 | C | ILE | A | 144 | 17.275 | −18.234 | 12.343 | 1.00 | 19.22 | 6 | C |
| ATOM | 1125 | O | ILE | A | 144 | 17.256 | −18.925 | 13.367 | 1.00 | 20.22 | 8 | O |
| ATOM | 1126 | CB | ILE | A | 144 | 19.369 | −17.639 | 11.102 | 1.00 | 17.51 | 6 | C |
| ATOM | 1127 | CG1 | ILE | A | 144 | 20.404 | −18.130 | 10.100 | 1.00 | 17.59 | 6 | C |
| ATOM | 1128 | CG2 | ILE | A | 144 | 19.998 | −17.530 | 12.493 | 1.00 | 18.52 | 6 | C |
| ATOM | 1129 | CD1 | ILE | A | 144 | 21.390 | −17.048 | 9.690 | 1.00 | 17.88 | 6 | C |
| ATOM | 1130 | N | LEU | A | 145 | 16.517 | −17.139 | 12.223 | 1.00 | 19.03 | 7 | N |
| ATOM | 1131 | CA | LEU | A | 145 | 15.599 | −16.715 | 13.270 | 1.00 | 18.79 | 6 | C |
| ATOM | 1132 | C | LEU | A | 145 | 14.612 | −17.849 | 13.566 | 1.00 | 19.52 | 6 | C |
| ATOM | 1133 | O | LEU | A | 145 | 14.344 | −18.141 | 14.730 | 1.00 | 20.05 | 8 | O |
| ATOM | 1134 | CB | LEU | A | 145 | 14.846 | −15.435 | 12.905 | 1.00 | 17.09 | 6 | C |
| ATOM | 1135 | CG | LEU | A | 145 | 15.716 | −14.169 | 12.814 | 1.00 | 16.79 | 6 | C |
| ATOM | 1136 | CD1 | LEU | A | 145 | 15.008 | −13.004 | 12.133 | 1.00 | 17.04 | 6 | C |
| ATOM | 1137 | CD2 | LEU | A | 145 | 16.115 | −13.742 | 14.227 | 1.00 | 16.63 | 6 | C |
| ATOM | 1138 | N | GLU | A | 146 | 14.075 | −18.485 | 12.522 | 1.00 | 20.35 | 7 | N |
| ATOM | 1139 | CA | GLU | A | 146 | 13.137 | −19.599 | 12.751 | 1.00 | 21.26 | 6 | C |
| ATOM | 1140 | C | GLU | A | 146 | 13.804 | −20.770 | 13.447 | 1.00 | 20.63 | 6 | C |
| ATOM | 1141 | O | GLU | A | 146 | 13.162 | −21.386 | 14.317 | 1.00 | 21.46 | 8 | O |
| ATOM | 1142 | CB | GLU | A | 146 | 12.470 | −20.015 | 11.435 | 1.00 | 21.41 | 6 | C |
| ATOM | 1143 | CG | GLU | A | 146 | 11.438 | −18.986 | 10.985 | 1.00 | 23.10 | 6 | C |
| ATOM | 1144 | CD | GLU | A | 146 | 10.736 | −19.275 | 9.679 | 1.00 | 24.14 | 6 | C |
| ATOM | 1145 | OE1 | GLU | A | 146 | 11.046 | −20.254 | 8.969 | 1.00 | 24.98 | 8 | O |
| ATOM | 1146 | OE2 | GLU | A | 146 | 9.831 | −18.473 | 9.346 | 1.00 | 23.46 | 8 | O |
| ATOM | 1147 | N | LYS | A | 147 | 15.048 | −21.097 | 13.150 | 1.00 | 20.86 | 7 | N |
| ATOM | 1148 | CA | LYS | A | 147 | 15.813 | −22.175 | 13.773 | 1.00 | 21.74 | 6 | C |
| ATOM | 1149 | C | LYS | A | 147 | 15.880 | −21.943 | 15.279 | 1.00 | 22.20 | 6 | C |
| ATOM | 1150 | O | LYS | A | 147 | 15.875 | −22.869 | 16.103 | 1.00 | 22.23 | 8 | O |
| ATOM | 1151 | CB | LYS | A | 147 | 17.217 | −22.285 | 13.160 | 1.00 | 22.16 | 6 | C |
| ATOM | 1152 | CG | LYS | A | 147 | 18.166 | −23.243 | 13.859 | 1.00 | 23.78 | 6 | C |
| ATOM | 1153 | CD | LYS | A | 147 | 19.637 | −22.913 | 13.675 | 1.00 | 25.60 | 6 | C |
| ATOM | 1154 | CE | LYS | A | 147 | 20.514 | −23.627 | 14.701 | 1.00 | 26.58 | 6 | C |
| ATOM | 1155 | NZ | LYS | A | 147 | 21.969 | −23.381 | 14.460 | 1.00 | 27.02 | 7 | N |
| ATOM | 1156 | N | HIS | A | 148 | 15.905 | −20.667 | 15.664 | 1.00 | 21.59 | 7 | N |
| ATOM | 1157 | CA | HIS | A | 148 | 15.949 | −20.248 | 17.050 | 1.00 | 22.17 | 6 | C |
| ATOM | 1158 | C | HIS | A | 148 | 14.593 | −19.888 | 17.612 | 1.00 | 22.33 | 6 | C |
| ATOM | 1159 | O | HIS | A | 148 | 14.527 | −19.315 | 18.720 | 1.00 | 20.28 | 8 | O |
| ATOM | 1160 | CB | HIS | A | 148 | 16.900 | −19.041 | 17.196 | 1.00 | 22.19 | 6 | C |
| ATOM | 1161 | CG | HIS | A | 148 | 18.307 | −19.517 | 16.952 | 1.00 | 22.86 | 6 | C |
| ATOM | 1162 | ND1 | HIS | A | 148 | 19.083 | −20.058 | 17.948 | 1.00 | 22.69 | 7 | N |
| ATOM | 1163 | CD2 | HIS | A | 148 | 19.051 | −19.534 | 15.829 | 1.00 | 22.82 | 6 | C |
| ATOM | 1164 | CE1 | HIS | A | 148 | 20.264 | −20.387 | 17.442 | 1.00 | 23.14 | 6 | C |
| ATOM | 1165 | NE2 | HIS | A | 148 | 20.265 | −20.076 | 16.160 | 1.00 | 23.72 | 7 | N |
| ATOM | 1166 | N | GLY | A | 149 | 13.478 | −20.264 | 16.994 | 1.00 | 22.29 | 7 | N |
| ATOM | 1167 | CA | GLY | A | 149 | 12.167 | −19.973 | 17.549 | 1.00 | 23.27 | 6 | C |
| ATOM | 1168 | C | GLY | A | 149 | 11.672 | −18.550 | 17.374 | 1.00 | 22.39 | 6 | C |
| ATOM | 1169 | O | GLY | A | 149 | 10.639 | −18.193 | 17.957 | 1.00 | 22.59 | 8 | O |
| ATOM | 1170 | N | LEU | A | 150 | 12.355 | −17.730 | 16.594 | 1.00 | 21.60 | 7 | N |
| ATOM | 1171 | CA | LEU | A | 150 | 11.908 | −16.364 | 16.354 | 1.00 | 22.15 | 6 | C |
| ATOM | 1172 | C | LEU | A | 150 | 11.232 | −16.311 | 14.985 | 1.00 | 22.27 | 6 | C |
| ATOM | 1173 | O | LEU | A | 150 | 11.243 | −17.307 | 14.247 | 1.00 | 22.47 | 8 | O |
| ATOM | 1174 | CB | LEU | A | 150 | 13.095 | −15.398 | 16.444 | 1.00 | 20.77 | 6 | C |
| ATOM | 1175 | CG | LEU | A | 150 | 13.781 | −15.424 | 17.822 | 1.00 | 19.86 | 6 | C |
| ATOM | 1176 | CD1 | LEU | A | 150 | 15.155 | −14.765 | 17.777 | 1.00 | 18.07 | 6 | C |
| ATOM | 1177 | CD2 | LEU | A | 150 | 12.875 | −14.733 | 18.827 | 1.00 | 21.34 | 6 | C |
| ATOM | 1178 | N | SER | A | 151 | 10.649 | −15.175 | 14.628 | 1.00 | 23.16 | 7 | N |
| ATOM | 1179 | CA | SER | A | 151 | 9.986 | −15.062 | 13.326 | 1.00 | 23.76 | 6 | C |
| ATOM | 1180 | C | SER | A | 151 | 10.484 | −13.850 | 12.555 | 1.00 | 22.85 | 6 | C |
| ATOM | 1181 | O | SER | A | 151 | 11.145 | −12.977 | 13.131 | 1.00 | 22.69 | 8 | O |
| ATOM | 1182 | CB | SER | A | 151 | 8.470 | −15.067 | 13.546 | 1.00 | 25.91 | 6 | C |
| ATOM | 1183 | OG | SER | A | 151 | 8.034 | −13.878 | 14.191 | 1.00 | 29.31 | 8 | O |
| ATOM | 1184 | N | GLY | A | 152 | 10.209 | −13.784 | 11.252 | 1.00 | 20.90 | 7 | N |
| ATOM | 1185 | CA | GLY | A | 152 | 10.662 | −12.648 | 10.459 | 1.00 | 19.94 | 6 | C |
| ATOM | 1186 | C | GLY | A | 152 | 9.821 | −12.500 | 9.196 | 1.00 | 20.29 | 6 | C |
| ATOM | 1187 | O | GLY | A | 152 | 9.175 | −13.470 | 8.818 | 1.00 | 21.01 | 8 | O |
| ATOM | 1188 | N | SER | A | 153 | 9.826 | −11.335 | 8.577 | 1.00 | 19.88 | 7 | N |
| ATOM | 1189 | CA | SER | A | 153 | 9.057 | −11.098 | 7.356 | 1.00 | 19.63 | 6 | C |
| ATOM | 1190 | C | SER | A | 153 | 9.761 | −10.031 | 6.531 | 1.00 | 20.62 | 6 | C |
| ATOM | 1191 | O | SER | A | 153 | 10.612 | −9.332 | 7.103 | 1.00 | 20.96 | 8 | O |
| ATOM | 1192 | CB | SER | A | 153 | 7.620 | −10.695 | 7.670 | 1.00 | 20.15 | 6 | C |
| ATOM | 1193 | OG | SER | A | 153 | 7.541 | −9.530 | 8.465 | 1.00 | 22.41 | 8 | O |
| ATOM | 1194 | N | VAL | A | 154 | 9.448 | −9.947 | 5.235 | 1.00 | 19.33 | 7 | N |
| ATOM | 1195 | CA | VAL | A | 154 | 10.106 | −8.947 | 4.398 | 1.00 | 18.92 | 6 | C |
| ATOM | 1196 | C | VAL | A | 154 | 9.185 | −7.753 | 4.170 | 1.00 | 18.31 | 6 | C |
| ATOM | 1197 | O | VAL | A | 154 | 7.986 | −7.945 | 3.947 | 1.00 | 17.21 | 8 | O |
| ATOM | 1198 | CB | VAL | A | 154 | 10.529 | −9.513 | 3.031 | 1.00 | 18.09 | 6 | C |
| ATOM | 1199 | CG1 | VAL | A | 154 | 11.146 | −8.484 | 2.101 | 1.00 | 18.36 | 6 | C |
| ATOM | 1200 | GG2 | VAL | A | 154 | 11.525 | −10.646 | 3.252 | 1.00 | 19.84 | 6 | C |
| ATOM | 1201 | N | VAL | A | 155 | 9.757 | −6.562 | 4.244 | 1.00 | 17.96 | 7 | N |
| ATOM | 1202 | CA | VAL | A | 155 | 9.034 | −5.323 | 4.004 | 1.00 | 18.14 | 6 | C |
| ATOM | 1203 | C | VAL | A | 155 | 9.639 | −4.712 | 2.739 | 1.00 | 19.37 | 6 | C |
| ATOM | 1204 | O | VAL | A | 155 | 10.862 | −4.567 | 2.658 | 1.00 | 18.84 | 8 | O |
| ATOM | 1205 | CB | VAL | A | 155 | 9.160 | −4.310 | 5.165 | 1.00 | 18.30 | 6 | C |
| ATOM | 1206 | CG1 | VAL | A | 155 | 8.474 | −2.991 | 4.810 | 1.00 | 17.05 | 6 | C |
| ATOM | 1207 | CG2 | VAL | A | 155 | 8.561 | −4.867 | 6.448 | 1.00 | 18.30 | 6 | C |
| ATOM | 1208 | N | ARG | A | 156 | 8.809 | −4.349 | 1.759 | 1.00 | 19.66 | 7 | N |
| ATOM | 1209 | CA | ARG | A | 156 | 9.348 | −3.765 | 0.533 | 1.00 | 20.17 | 6 | C |
| ATOM | 1210 | C | ARG | A | 156 | 9.583 | −2.269 | 0.705 | 1.00 | 18.82 | 6 | C |
| ATOM | 1211 | O | ARG | A | 156 | 8.747 | −1.622 | 1.353 | 1.00 | 18.51 | 8 | O |
| ATOM | 1212 | CB | ARG | A | 156 | 8.349 | −4.055 | −0.601 | 1.00 | 21.10 | 6 | C |
| ATOM | 1213 | CG | ARG | A | 156 | 7.900 | −5.511 | −0.675 | 1.00 | 20.86 | 6 | C |
| ATOM | 1214 | CD | ARG | A | 156 | 9.049 | −6.490 | −0.936 | 1.00 | 21.44 | 6 | C |
| ATOM | 1215 | NE | ARG | A | 156 | 8.595 | −7.859 | −0.699 | 1.00 | 21.21 | 7 | N |
| ATOM | 1216 | CZ | ARG | A | 156 | 9.175 | −9.005 | −0.969 | 1.00 | 21.71 | 6 | C |
| ATOM | 1217 | NH1 | ARG | A | 156 | 10.369 | −9.062 | −1.552 | 1.00 | 21.53 | 7 | N |
| ATOM | 1218 | NH2 | ARG | A | 156 | 8.560 | −10.141 | −0.653 | 1.00 | 22.03 | 7 | N |
| ATOM | 1219 | N | LEU | A | 157 | 10.666 | −1.722 | 0.173 | 1.00 | 18.60 | 7 | N |
| ATOM | 1220 | CA | LEU | A | 157 | 10.915 | −0.283 | 0.303 | 1.00 | 19.61 | 6 | C |
| ATOM | 1221 | C | LEU | A | 157 | 11.612 | 0.248 | −0.935 | 1.00 | 21.59 | 6 | C |
| ATOM | 1222 | O | LEU | A | 157 | 12.235 | −0.500 | −1.690 | 1.00 | 22.60 | 8 | O |
| ATOM | 1223 | CB | LEU | A | 157 | 11.703 | 0.053 | −1.580 | 1.00 | 19.45 | 6 | C |
| ATOM | 1224 | CG | LEU | A | 157 | 13.130 | −0.477 | 1.688 | 1.00 | 19.36 | 6 | C |
| ATOM | 1225 | CD1 | LEU | A | 157 | 14.137 | 0.419 | 0.986 | 1.00 | 21.00 | 6 | C |
| ATOM | 1226 | CD2 | LEU | A | 157 | 13.502 | −0.642 | 3.169 | 1.00 | 19.57 | 6 | C |
| ATOM | 1227 | N | GLU | A | 158 | 11.521 | 1.554 | −1.203 | 1.00 | 23.99 | 7 | N |
| ATOM | 1228 | CA | GLU | A | 158 | 12.171 | 2.090 | −2.394 | 1.00 | 25.75 | 6 | C |
| ATOM | 1229 | C | GLU | A | 158 | 12.818 | 3.449 | −2.151 | 1.00 | 25.61 | 6 | C |
| ATOM | 1230 | O | GLU | A | 158 | 13.796 | 3.770 | −2.829 | 1.00 | 24.68 | 8 | O |
| ATOM | 1231 | CB | GLU | A | 158 | 11.183 | 2.225 | −3.555 | 1.00 | 28.27 | 6 | C |
| ATOM | 1232 | CG | GLU | A | 158 | 10.687 | 0.989 | −4.260 | 1.00 | 31.70 | 6 | C |
| ATOM | 1233 | CD | GLU | A | 158 | 11.727 | 0.040 | −4.795 | 1.00 | 34.04 | 6 | C |
| ATOM | 1234 | OE1 | GLU | A | 158 | 12.642 | 0.505 | −5.513 | 1.00 | 36.03 | 8 | O |
| ATOM | 1235 | OE2 | GLU | A | 158 | 11.681 | −1.184 | −4.529 | 1.00 | 36.26 | 8 | O |
| ATOM | 1236 | N | LYS | A | 159 | 12.290 | 4.225 | −1.217 | 1.00 | 24.87 | 7 | N |
| ATOM | 1237 | CA | LYS | A | 159 | 12.784 | 5.563 | −0.940 | 1.00 | 24.95 | 6 | C |
| ATOM | 1238 | C | LYS | A | 159 | 13.810 | 5.799 | 0.147 | 1.00 | 25.09 | 6 | C |
| ATOM | 1239 | O | LYS | A | 159 | 14.260 | 6.966 | 0.296 | 1.00 | 25.42 | 8 | O |
| ATOM | 1240 | CB | LYS | A | 159 | 11.548 | 6.453 | −0.654 | 1.00 | 26.06 | 6 | C |
| ATOM | 1241 | CG | LYS | A | 159 | 10.715 | 6.745 | −1.903 | 1.00 | 28.38 | 6 | C |
| ATOM | 1242 | CD | LYS | A | 159 | 9.389 | 7.398 | −1.513 | 1.00 | 28.90 | 6 | C |
| ATOM | 1243 | CE | LYS | A | 159 | 8.596 | 7.818 | −2.739 | 1.00 | 30.95 | 6 | C |
| ATOM | 1244 | NZ | LYS | A | 159 | 7.288 | 8.442 | −2.350 | 1.00 | 32.67 | 7 | N |
| ATOM | 1245 | N | VAL | A | 160 | 14.221 | 4.827 | 0.948 | 1.00 | 22.91 | 7 | N |
| ATOM | 1246 | CA | VAL | A | 160 | 15.217 | 5.064 | 1.987 | 1.00 | 22.21 | 6 | C |
| ATOM | 1247 | C | VAL | A | 160 | 16.401 | 4.105 | 1.906 | 1.00 | 22.17 | 6 | C |
| ATOM | 1248 | O | VAL | A | 160 | 16.318 | 3.079 | 1.242 | 1.00 | 21.48 | 8 | O |
| ATOM | 1249 | CB | VAL | A | 160 | 14.625 | 4.956 | 3.403 | 1.00 | 23.01 | 6 | C |
| ATOM | 1250 | CG1 | VAL | A | 160 | 13.808 | 6.203 | 3.738 | 1.00 | 23.23 | 6 | C |
| ATOM | 1251 | CG2 | VAL | A | 160 | 13.767 | 3.697 | 3.517 | 1.00 | 23.14 | 6 | C |
| ATOM | 1252 | N | LEU | A | 161 | 17.486 | 4.458 | 2.607 | 1.00 | 20.56 | 7 | N |
| ATOM | 1253 | CA | LEU | A | 161 | 18.672 | 3.614 | 2.601 | 1.00 | 19.22 | 6 | C |
| ATOM | 1254 | C | LEU | A | 161 | 18.402 | 2.279 | 3.280 | 1.00 | 17.13 | 6 | C |
| ATOM | 1255 | O | LEU | A | 161 | 18.745 | 1.240 | 2.755 | 1.00 | 16.00 | 8 | O |
| ATOM | 1256 | CB | LEU | A | 161 | 19.829 | 4.329 | 3.330 | 1.00 | 19.43 | 6 | C |
| ATOM | 1257 | CG | LEU | A | 161 | 21.153 | 3.557 | 3.341 | 1.00 | 19.69 | 6 | C |
| ATOM | 1258 | CD1 | LEU | A | 161 | 21.689 | 3.334 | 1.939 | 1.00 | 20.11 | 6 | C |
| ATOM | 1259 | CD2 | LEU | A | 161 | 22.169 | 4.308 | 4.207 | 1.00 | 20.46 | 6 | C |
| ATOM | 1260 | N | HIS | A | 162 | 17.806 | 2.310 | 4.462 | 1.00 | 17.28 | 7 | N |
| ATOM | 1261 | CA | HIS | A | 162 | 17.487 | 1.115 | 5.217 | 1.00 | 16.55 | 6 | C |
| ATOM | 1262 | C | HIS | A | 162 | 16.087 | 1.293 | 5.811 | 1.00 | 15.34 | 6 | C |
| ATOM | 1263 | O | HIS | A | 162 | 15.646 | 2.423 | 5.982 | 1.00 | 16.70 | 8 | O |
| ATOM | 1264 | CB | HIS | A | 162 | 18.381 | 0.868 | 6.433 | 1.00 | 16.97 | 6 | C |
| ATOM | 1265 | CG | HIS | A | 162 | 19.827 | 0.597 | 6.172 | 1.00 | 18.30 | 6 | C |
| ATOM | 1266 | ND1 | HIS | A | 162 | 20.807 | 1.541 | 6.390 | 1.00 | 18.05 | 7 | N |
| ATOM | 1267 | CD2 | HIS | A | 162 | 20.442 | −0.502 | 5.706 | 1.00 | 17.90 | 6 | C |
| ATOM | 1268 | CE1 | HIS | A | 162 | 21.982 | 1.018 | 6.063 | 1.00 | 18.64 | 6 | C |
| ATOM | 1269 | NE2 | HIS | A | 162 | 21.794 | −0.215 | 5.650 | 1.00 | 18.25 | 7 | N |
| ATOM | 1270 | N | LEU | A | 163 | 15.450 | 0.194 | 6.160 | 1.00 | 13.08 | 7 | N |
| ATOM | 1271 | CA | LEU | A | 163 | 14.131 | 0.263 | 6.762 | 1.00 | 14.25 | 6 | C |
| ATOM | 1272 | C | LEU | A | 163 | 14.055 | 1.220 | 7.943 | 1.00 | 16.22 | 6 | C |
| ATOM | 1273 | O | LEU | A | 163 | 13.177 | 2.075 | 8.026 | 1.00 | 14.46 | 8 | O |
| ATOM | 1274 | CB | LEU | A | 163 | 13.766 | −1.146 | 7.240 | 1.00 | 15.67 | 6 | C |
| ATOM | 1275 | CG | LEU | A | 163 | 12.357 | −1.344 | 7.807 | 1.00 | 16.19 | 6 | C |
| ATOM | 1276 | CD1 | LEU | A | 163 | 11.348 | −1.447 | 6.671 | 1.00 | 16.96 | 6 | C |
| ATOM | 1277 | CD2 | LEU | A | 163 | 12.327 | −2.607 | 8.653 | 1.00 | 15.98 | 6 | C |
| ATOM | 1278 | N | LYS | A | 164 | 15.022 | 1.116 | 8.882 | 1.00 | 16.05 | 7 | N |
| ATOM | 1279 | CA | LYS | A | 164 | 15.027 | 1.937 | 10.081 | 1.00 | 17.07 | 6 | C |
| ATOM | 1280 | C | LYS | A | 164 | 15.381 | 3.394 | 9.914 | 1.00 | 17.42 | 6 | C |
| ATOM | 1281 | O | LYS | A | 164 | 15.346 | 4.178 | 10.871 | 1.00 | 18.48 | 8 | O |
| ATOM | 1282 | CB | LYS | A | 164 | 15.796 | 1.275 | 11.235 | 1.00 | 16.84 | 6 | C |
| ATOM | 1283 | CG | LYS | A | 164 | 15.027 | 0.051 | 11.726 | 1.00 | 17.41 | 6 | C |
| ATOM | 1284 | CD | LYS | A | 164 | 15.448 | −0.385 | 13.117 | 1.00 | 17.20 | 6 | C |
| ATOM | 1285 | CE | LYS | A | 164 | 16.927 | −0.767 | 13.181 | 1.00 | 19.09 | 6 | C |
| ATOM | 1286 | NZ | LYS | A | 164 | 17.151 | −1.717 | 14.319 | 1.00 | 18.11 | 7 | N |
| ATOM | 1287 | N | THR | A | 165 | 15.625 | 3.837 | 8.696 | 1.00 | 17.74 | 7 | N |
| ATOM | 1288 | CA | THR | A | 165 | 15.844 | 5.232 | 8.353 | 1.00 | 17.32 | 6 | C |
| ATOM | 1289 | C | THR | A | 165 | 14.511 | 5.950 | 8.601 | 1.00 | 17.49 | 6 | C |
| ATOM | 1290 | O | THR | A | 165 | 14.487 | 7.128 | 8.983 | 1.00 | 14.86 | 8 | O |
| ATOM | 1291 | CB | THR | A | 165 | 16.194 | 5.399 | 6.873 | 1.00 | 18.49 | 6 | C |
| ATOM | 1292 | OG1 | THR | A | 165 | 17.520 | 4.891 | 6.613 | 1.00 | 19.17 | 8 | O |
| ATOM | 1293 | CG2 | THR | A | 165 | 16.120 | 6.840 | 6.393 | 1.00 | 19.64 | 6 | C |
| ATOM | 1294 | N | GLY | A | 166 | 13.415 | 5.239 | 8.320 | 1.00 | 16.16 | 7 | N |
| ATOM | 1295 | CA | GLY | A | 166 | 12.092 | 5.814 | 8.486 | 1.00 | 17.61 | 6 | C |
| ATOM | 1296 | C | GLY | A | 166 | 11.169 | 5.097 | 9.428 | 1.00 | 18.31 | 6 | C |
| ATOM | 1297 | O | GLY | A | 166 | 9.936 | 5.267 | 9.387 | 1.00 | 19.06 | 8 | O |
| ATOM | 1298 | N | LEU | A | 167 | 11.723 | 4.292 | 10.351 | 1.00 | 17.78 | 7 | N |
| ATOM | 1299 | CA | LEU | A | 167 | 10.872 | 3.568 | 11.284 | 1.00 | 17.81 | 6 | C |
| ATOM | 1300 | C | LEU | A | 167 | 11.630 | 3.274 | 12.590 | 1.00 | 17.52 | 6 | C |
| ATOM | 1301 | O | LEU | A | 167 | 12.848 | 3.105 | 12.559 | 1.00 | 17.11 | 8 | O |
| ATOM | 1302 | CB | LEU | A | 167 | 10.513 | 2.199 | 10.718 | 1.00 | 19.35 | 6 | C |
| ATOM | 1303 | CG | LEU | A | 167 | 9.354 | 2.001 | 9.754 | 1.00 | 19.43 | 6 | C |
| ATOM | 1304 | CD1 | LEU | A | 167 | 9.493 | 0.637 | 9.090 | 1.00 | 20.42 | 6 | C |
| ATOM | 1305 | CD2 | LEU | A | 167 | 8.043 | 2.133 | 10.521 | 1.00 | 19.61 | 6 | C |
| ATOM | 1306 | N | ALA | A | 168 | 10.879 | 3.186 | 13.658 | 1.00 | 17.23 | 7 | N |
| ATOM | 1307 | CA | ALA | A | 168 | 11.447 | 2.891 | 14.967 | 1.00 | 17.83 | 6 | C |
| ATOM | 1308 | C | ALA | A | 168 | 10.382 | 2.350 | 15.908 | 1.00 | 18.02 | 6 | C |
| ATOM | 1309 | O | ALA | A | 168 | 9.362 | 2.997 | 16.129 | 1.00 | 18.31 | 8 | O |
| ATOM | 1310 | CB | ALA | A | 168 | 12.084 | 4.112 | 15.618 | 1.00 | 17.48 | 6 | C |
| ATOM | 1311 | N | TYR | A | 169 | 10.648 | 1.179 | 16.478 | 1.00 | 17.69 | 7 | N |
| ATOM | 1312 | CA | TYR | A | 169 | 9.700 | 0.592 | 17.428 | 1.00 | 17.99 | 6 | C |
| ATOM | 1313 | C | TYR | A | 169 | 10.115 | 0.962 | 18.841 | 1.00 | 18.41 | 6 | C |
| ATOM | 1314 | O | TYR | A | 169 | 11.250 | 0.709 | 19.234 | 1.00 | 17.21 | 8 | O |
| ATOM | 1315 | CB | TYR | A | 169 | 9.607 | −0.924 | 17.258 | 1.00 | 18.47 | 6 | C |
| ATOM | 1316 | CG | TYR | A | 169 | 8.578 | −1.564 | 18.173 | 1.00 | 19.18 | 6 | C |
| ATOM | 1317 | CD1 | TYR | A | 169 | 7.248 | −1.187 | 18.114 | 1.00 | 20.54 | 6 | C |
| ATOM | 1318 | CD2 | TYR | A | 169 | 8.929 | −2.541 | 19.093 | 1.00 | 19.63 | 6 | C |
| ATOM | 1319 | CE1 | TYR | A | 169 | 6.293 | −1.755 | 18.931 | 1.00 | 20.41 | 6 | C |
| ATOM | 1320 | CE2 | TYR | A | 169 | 7.993 | −3.121 | 19.924 | 1.00 | 20.25 | 6 | C |
| ATOM | 1321 | CZ | TYR | A | 169 | 6.675 | −2.725 | 19.837 | 1.00 | 21.08 | 6 | C |
| ATOM | 1322 | OH | TYR | A | 169 | 5.730 | −3.300 | 20.661 | 1.00 | 20.59 | 8 | O |
| ATOM | 1323 | N | LEU | A | 170 | 9.208 | 1.593 | 19.598 | 1.00 | 18.68 | 7 | N |
| ATOM | 1324 | CA | LEU | A | 170 | 9.503 | 2.021 | 20.954 | 1.00 | 19.37 | 6 | C |
| ATOM | 1325 | C | LEU | A | 170 | 8.857 | 1.184 | 22.048 | 1.00 | 20.39 | 6 | C |
| ATOM | 1326 | O | LEU | A | 170 | 8.613 | 1.651 | 23.169 | 1.00 | 21.84 | 8 | O |
| ATOM | 1327 | CB | LEU | A | 170 | 9.075 | 3.491 | 21.111 | 1.00 | 19.37 | 6 | C |
| ATOM | 1328 | CG | LEU | A | 170 | 9.532 | 4.481 | 20.033 | 1.00 | 19.60 | 6 | C |
| ATOM | 1329 | CD1 | LEU | A | 170 | 8.998 | 5.876 | 20.331 | 1.00 | 19.10 | 6 | C |
| ATOM | 1330 | CD2 | LEU | A | 170 | 11.051 | 4.496 | 19.910 | 1.00 | 18.71 | 6 | C |
| ATOM | 1331 | N | GLU | A | 171 | 8.588 | −0.081 | 21.770 | 1.00 | 20.99 | 7 | N |
| ATOM | 1332 | CA | GLU | A | 171 | 7.960 | −1.007 | 22.703 | 1.00 | 21.35 | 6 | C |
| ATOM | 1333 | C | GLU | A | 171 | 6.538 | −0.532 | 22.967 | 1.00 | 22.12 | 6 | C |
| ATOM | 1334 | O | GLU | A | 171 | 6.145 | 0.578 | 22.604 | 1.00 | 21.68 | 8 | O |
| ATOM | 1335 | CB | GLU | A | 171 | 8.752 | −1.140 | 24.004 | 1.00 | 21.46 | 6 | C |
| ATOM | 1336 | CG | GLU | A | 171 | 10.123 | −1.773 | 23.848 | 1.00 | 20.52 | 6 | C |
| ATOM | 1337 | CD | GLU | A | 171 | 10.138 | −3.040 | 23.014 | 1.00 | 21.83 | 6 | C |
| ATOM | 1338 | OE1 | GLU | A | 171 | 9.359 | −3.973 | 23.300 | 1.00 | 22.63 | 8 | O |
| ATOM | 1339 | OE2 | GLU | A | 171 | 10.932 | −3.116 | 22.065 | 1.00 | 20.98 | 8 | O |
| ATOM | 1340 | N | HIS | A | 172 | 5.713 | −1.356 | 23.599 | 1.00 | 23.36 | 7 | N |
| ATOM | 1341 | CA | HIS | A | 172 | 4.337 | −1.055 | 23.937 | 1.00 | 23.51 | 6 | C |
| ATOM | 1342 | C | HIS | A | 172 | 3.433 | −0.728 | 22.755 | 1.00 | 23.94 | 6 | C |
| ATOM | 1343 | O | HIS | A | 172 | 2.443 | −0.005 | 22.909 | 1.00 | 24.55 | 8 | O |
| ATOM | 1344 | CB | HIS | A | 172 | 4.250 | 0.077 | 24.980 | 1.00 | 23.95 | 6 | C |
| ATOM | 1345 | CG | HIS | A | 172 | 5.203 | −0.126 | 26.124 | 1.00 | 25.24 | 6 | C |
| ATOM | 1346 | ND1 | HIS | A | 172 | 5.097 | −1.149 | 27.036 | 1.00 | 26.05 | 7 | N |
| ATOM | 1347 | CD2 | HIS | A | 172 | 6.303 | 0.590 | 26.470 | 1.00 | 25.34 | 6 | C |
| ATOM | 1348 | CE1 | HIS | A | 172 | 6.091 | −1.056 | 27.907 | 1.00 | 25.92 | 6 | C |
| ATOM | 1349 | NE2 | HIS | A | 172 | 6.840 | −0.007 | 27.584 | 1.00 | 25.89 | 7 | N |
| ATOM | 1350 | N | ASN | A | 173 | 3.705 | −1.223 | 21.569 | 1.00 | 23.62 | 7 | N |
| ATOM | 1351 | CA | ASN | A | 173 | 2.948 | −1.029 | 20.353 | 1.00 | 24.31 | 6 | C |
| ATOM | 1352 | C | ASN | A | 173 | 3.089 | 0.358 | 19.747 | 1.00 | 24.56 | 6 | C |
| ATOM | 1353 | O | ASN | A | 173 | 2.354 | 0.693 | 18.815 | 1.00 | 24.91 | 8 | O |
| ATOM | 1354 | CB | ASN | A | 173 | 1.453 | −1.353 | 20.586 | 1.00 | 23.82 | 6 | C |
| ATOM | 1355 | CG | ASN | A | 173 | 0.776 | −1.845 | 19.323 | 1.00 | 24.19 | 6 | C |
| ATOM | 1356 | OD1 | ASN | A | 173 | 1.351 | −2.612 | 18.551 | 1.00 | 23.12 | 8 | O |
| ATOM | 1357 | ND2 | ASN | A | 173 | −0.459 | −1.428 | 19.064 | 1.00 | 23.58 | 7 | N |
| ATOM | 1358 | N | ASN | A | 174 | 4.028 | 1.174 | 20.222 | 1.00 | 23.00 | 7 | N |
| ATOM | 1359 | CA | ASN | A | 174 | 4.219 | 2.524 | 19.707 | 1.00 | 22.14 | 6 | C |
| ATOM | 1360 | C | ASN | A | 174 | 5.253 | 2.488 | 18.590 | 1.00 | 22.53 | 6 | C |
| ATOM | 1361 | O | ASN | A | 174 | 6.390 | 2.115 | 18.885 | 1.00 | 22.39 | 8 | O |
| ATOM | 1362 | CB | ASN | A | 174 | 4.663 | 3.476 | 20.826 | 1.00 | 21.92 | 6 | C |
| ATOM | 1363 | CG | ASN | A | 174 | 3.589 | 3.587 | 21.891 | 1.00 | 21.52 | 6 | C |
| ATOM | 1364 | OD1 | ASN | A | 174 | 2.411 | 3.660 | 21.516 | 1.00 | 23.82 | 8 | O |
| ATOM | 1365 | ND2 | ASN | A | 174 | 3.908 | 3.602 | 23.173 | 1.00 | 20.48 | 7 | N |
| ATOM | 1366 | N | LEU | A | 175 | 4.844 | 2.832 | 17.378 | 1.00 | 20.45 | 7 | N |
| ATOM | 1367 | CA | LEU | A | 175 | 5.739 | 2.805 | 16.234 | 1.00 | 19.21 | 6 | C |
| ATOM | 1368 | C | LEU | A | 175 | 5.958 | 4.158 | 15.572 | 1.00 | 19.72 | 6 | C |
| ATOM | 1369 | O | LEU | A | 175 | 4.981 | 4.777 | 15.139 | 1.00 | 20.59 | 8 | O |
| ATOM | 1370 | CB | LEU | A | 175 | 5.141 | 1.847 | 15.180 | 1.00 | 18.84 | 6 | C |
| ATOM | 1371 | CG | LEU | A | 175 | 5.948 | 1.646 | 13.890 | 1.00 | 18.45 | 6 | C |
| ATOM | 1372 | CD1 | LEU | A | 175 | 7.272 | 0.938 | 14.188 | 1.00 | 18.48 | 6 | C |
| ATOM | 1373 | CD2 | LEU | A | 175 | 5.162 | 0.843 | 12.860 | 1.00 | 18.10 | 6 | C |
| ATOM | 1374 | N | LEU | A | 176 | 7.204 | 4.608 | 15.440 | 1.00 | 18.64 | 7 | N |
| ATOM | 1375 | CA | LEU | A | 176 | 7.458 | 5.866 | 14.727 | 1.00 | 19.35 | 6 | C |
| ATOM | 1376 | C | LEU | A | 176 | 7.494 | 5.524 | 13.229 | 1.00 | 19.38 | 6 | C |
| ATOM | 1377 | O | LEU | A | 176 | 8.128 | 4.542 | 12.825 | 1.00 | 18.01 | 8 | O |
| ATOM | 1378 | CB | LEU | A | 176 | 8.801 | 6.515 | 15.084 | 1.00 | 19.79 | 6 | C |
| ATOM | 1379 | CG | LEU | A | 176 | 9.032 | 6.919 | 16.547 | 1.00 | 20.47 | 6 | C |
| ATOM | 1380 | CD1 | LEU | A | 176 | 10.393 | 7.591 | 16.697 | 1.00 | 20.30 | 6 | C |
| ATOM | 1381 | CD2 | LEU | A | 176 | 7.925 | 7.851 | 17.035 | 1.00 | 19.69 | 6 | C |
| ATOM | 1382 | N | ALA | A | 177 | 6.812 | 6.297 | 12.387 | 1.00 | 19.88 | 7 | N |
| ATOM | 1383 | CA | ALA | A | 177 | 6.824 | 6.021 | 10.964 | 1.00 | 20.22 | 6 | C |
| ATOM | 1384 | C | ALA | A | 177 | 6.810 | 7.309 | 10.143 | 1.00 | 20.22 | 6 | C |
| ATOM | 1385 | O | ALA | A | 177 | 6.121 | 8.277 | 10.447 | 1.00 | 19.81 | 8 | O |
| ATOM | 1386 | CB | ALA | A | 177 | 5.671 | 5.142 | 10.508 | 1.00 | 20.62 | 6 | C |
| ATOM | 1387 | N | ALA | A | 178 | 7.632 | 7.266 | 9.100 | 1.00 | 20.05 | 7 | N |
| ATOM | 1388 | CA | ALA | A | 178 | 7.777 | 8.342 | 8.147 | 1.00 | 21.94 | 6 | C |
| ATOM | 1389 | C | ALA | A | 178 | 7.690 | 7.808 | 6.719 | 1.00 | 22.56 | 6 | C |
| ATOM | 1390 | O | ALA | A | 178 | 7.922 | 6.639 | 6.424 | 1.00 | 22.87 | 8 | O |
| ATOM | 1391 | CB | ALA | A | 178 | 9.128 | 9.047 | 8.269 | 1.00 | 21.64 | 6 | C |
| ATOM | 1392 | N | GLY | A | 179 | 7.371 | 8.728 | 5.814 | 1.00 | 22.74 | 7 | N |
| ATOM | 1393 | CA | GLY | A | 179 | 7.282 | 8.459 | 4.407 | 1.00 | 22.62 | 6 | C |
| ATOM | 1394 | C | GLY | A | 179 | 6.397 | 7.296 | 4.011 | 1.00 | 22.42 | 6 | C |
| ATOM | 1395 | O | GLY | A | 179 | 5.243 | 7.177 | 4.406 | 1.00 | 20.38 | 8 | O |
| ATOM | 1396 | N | GLU | A | 180 | 6.979 | 6.396 | 3.209 | 1.00 | 22.46 | 7 | N |
| ATOM | 1397 | CA | GLU | A | 180 | 6.254 | 5.243 | 2.701 | 1.00 | 23.16 | 6 | C |
| ATOM | 1398 | C | GLU | A | 180 | 5.864 | 4.178 | 3.706 | 1.00 | 23.00 | 6 | C |
| ATOM | 1399 | O | GLU | A | 180 | 5.105 | 3.282 | 3.318 | 1.00 | 22.71 | 8 | O |
| ATOM | 1400 | CB | GLU | A | 180 | 7.107 | 4.614 | 1.585 | 1.00 | 22.99 | 6 | C |
| ATOM | 1401 | CG | GLU | A | 180 | 8.286 | 3.820 | 2.114 | 1.00 | 22.26 | 6 | C |
| ATOM | 1402 | CD | GLU | A | 180 | 9.349 | 3.515 | 1.095 | 1.00 | 21.60 | 6 | C |
| ATOM | 1403 | OE1 | GLU | A | 180 | 9.182 | 2.631 | 0.229 | 1.00 | 21.67 | 8 | O |
| ATOM | 1404 | OE2 | GLU | A | 180 | 10.406 | 4.178 | 1.150 | 1.00 | 22.58 | 8 | O |
| ATOM | 1405 | N | PHE | A | 181 | 6.328 | 4.210 | 4.956 | 1.00 | 22.28 | 7 | N |
| ATOM | 1406 | CA | PHE | A | 181 | 5.945 | 3.154 | 5.889 | 1.00 | 21.26 | 6 | C |
| ATOM | 1407 | C | PHE | A | 181 | 4.637 | 3.455 | 6.606 | 1.00 | 22.40 | 6 | C |
| ATOM | 1408 | O | PHE | A | 181 | 3.963 | 2.538 | 7.082 | 1.00 | 23.21 | 8 | O |
| ATOM | 1409 | CB | PHE | A | 181 | 7.080 | 2.899 | 6.895 | 1.00 | 20.41 | 6 | C |
| ATOM | 1410 | CG | PHE | A | 181 | 8.367 | 2.567 | 6.188 | 1.00 | 19.24 | 6 | C |
| ATOM | 1411 | CD1 | PHE | A | 181 | 9.443 | 3.427 | 6.258 | 1.00 | 18.10 | 6 | C |
| ATOM | 1412 | CD2 | PHE | A | 181 | 8.492 | 1.395 | 5.451 | 1.00 | 18.47 | 6 | C |
| ATOM | 1413 | CE1 | PHE | A | 181 | 10.626 | 3.130 | 5.608 | 1.00 | 17.52 | 6 | C |
| ATOM | 1414 | CE2 | PHE | A | 181 | 9.674 | 1.093 | 4.801 | 1.00 | 17.24 | 6 | C |
| ATOM | 1415 | CZ | PHE | A | 181 | 10.740 | 1.965 | 4.879 | 1.00 | 17.01 | 6 | C |
| ATOM | 1416 | N | VAL | A | 182 | 4.258 | 4.730 | 6.678 | 1.00 | 22.10 | 7 | N |
| ATOM | 1417 | CA | VAL | A | 182 | 3.050 | 5.147 | 7.367 | 1.00 | 23.74 | 6 | C |
| ATOM | 1418 | C | VAL | A | 182 | 1.781 | 4.401 | 7.009 | 1.00 | 25.08 | 6 | C |
| ATOM | 1419 | O | VAL | A | 182 | 1.006 | 4.038 | 7.904 | 1.00 | 26.18 | 8 | O |
| ATOM | 1420 | CB | VAL | A | 182 | 2.821 | 6.668 | 7.166 | 1.00 | 23.39 | 6 | C |
| ATOM | 1421 | CG1 | VAL | A | 182 | 1.560 | 7.159 | 7.872 | 1.00 | 22.43 | 6 | C |
| ATOM | 1422 | CG2 | VAL | A | 182 | 4.015 | 7.461 | 7.663 | 1.00 | 22.67 | 6 | C |
| ATOM | 1423 | N | SER | A | 183 | 1.499 | 4.177 | 5.735 | 1.00 | 26.65 | 7 | N |
| ATOM | 1424 | CA | SER | A | 183 | 0.278 | 3.498 | 5.325 | 1.00 | 28.00 | 6 | C |
| ATOM | 1425 | C | SER | A | 183 | 0.435 | 2.023 | 5.002 | 1.00 | 27.48 | 6 | C |
| ATOM | 1426 | O | SER | A | 183 | −0.455 | 1.450 | 4.362 | 1.00 | 27.48 | 8 | O |
| ATOM | 1427 | CB | SER | A | 183 | −0.281 | 4.207 | 4.078 | 1.00 | 29.10 | 6 | C |
| ATOM | 1428 | OG | SER | A | 183 | −0.910 | 5.434 | 4.424 | 1.00 | 31.54 | 8 | O |
| ATOM | 1429 | N | LYS | A | 184 | 1.523 | 1.385 | 5.405 | 1.00 | 26.59 | 7 | N |
| ATOM | 1430 | CA | LYS | A | 184 | 1.705 | −0.032 | 5.112 | 1.00 | 26.12 | 6 | C |
| ATOM | 1431 | C | LYS | A | 184 | 0.703 | −0.888 | 5.866 | 1.00 | 25.33 | 6 | C |
| ATOM | 1432 | O | LYS | A | 184 | 0.558 | −0.709 | 7.065 | 1.00 | 25.76 | 8 | O |
| ATOM | 1433 | CB | LYS | A | 184 | 3.081 | −0.519 | 5.599 | 1.00 | 25.91 | 6 | C |
| ATOM | 1434 | CG | LYS | A | 184 | 4.217 | 0.075 | 4.789 | 1.00 | 26.00 | 6 | C |
| ATOM | 1435 | CD | LYS | A | 184 | 4.432 | −0.765 | 3.540 | 1.00 | 25.50 | 6 | C |
| ATOM | 1436 | CE | LYS | A | 184 | 5.764 | −0.421 | 2.889 | 1.00 | 25.43 | 6 | C |
| ATOM | 1437 | NZ | LYS | A | 184 | 5.959 | −1.196 | 1.631 | 1.00 | 25.69 | 7 | N |
| ATOM | 1438 | N | PRO | A | 185 | 0.073 | −1.825 | 5.178 | 1.00 | 24.81 | 7 | N |
| ATOM | 1439 | CA | PRO | A | 185 | −0.888 | −2.725 | 5.790 | 1.00 | 24.53 | 6 | C |
| ATOM | 1440 | C | PRO | A | 185 | −0.304 | −3.505 | 6.953 | 1.00 | 24.13 | 6 | C |
| ATOM | 1441 | O | PRO | A | 185 | −1.005 | −3.704 | 7.949 | 1.00 | 23.04 | 8 | O |
| ATOM | 1442 | CB | PRO | A | 185 | −1.316 | −3.663 | 4.670 | 1.00 | 24.58 | 6 | C |
| ATOM | 1443 | CG | PRO | A | 185 | −1.003 | −2.921 | 3.414 | 1.00 | 25.72 | 6 | C |
| ATOM | 1444 | CD | PRO | A | 185 | 0.221 | −2.093 | 3.728 | 1.00 | 24.93 | 6 | C |
| ATOM | 1445 | N | GLU | A | 186 | 0.955 | −3.940 | 6.847 | 1.00 | 24.97 | 7 | N |
| ATOM | 1446 | CA | GLU | A | 186 | 1.592 | −4.707 | 7.909 | 1.00 | 24.48 | 6 | C |
| ATOM | 1447 | C | GLU | A | 186 | 1.730 | −3.937 | 9.224 | 1.00 | 23.93 | 6 | C |
| ATOM | 1448 | O | GLU | A | 186 | 1.914 | −4.585 | 10.261 | 1.00 | 22.58 | 8 | O |
| ATOM | 1449 | CB | GLU | A | 186 | 3.010 | −5.126 | 7.594 | 1.00 | 26.12 | 6 | C |
| ATOM | 1450 | CG | GLU | A | 186 | 3.757 | −5.491 | 6.394 | 1.00 | 27.68 | 6 | C |
| ATOM | 1451 | CD | GLU | A | 186 | 3.980 | −4.455 | 5.322 | 1.00 | 27.82 | 6 | C |
| ATOM | 1452 | OE1 | GLU | A | 186 | 5.109 | −4.276 | 4.816 | 1.00 | 27.28 | 8 | O |
| ATOM | 1453 | OE2 | GLU | A | 186 | 3.000 | −3.777 | 4.933 | 1.00 | 26.89 | 8 | O |
| ATOM | 1454 | N | PHE | A | 187 | 1.724 | −2.607 | 9.188 | 1.00 | 23.09 | 7 | N |
| ATOM | 1455 | CA | PHE | A | 187 | 1.909 | −1.841 | 10.418 | 1.00 | 23.45 | 6 | C |
| ATOM | 1456 | C | PHE | A | 187 | 0.641 | −1.187 | 10.957 | 1.00 | 24.14 | 6 | C |
| ATOM | 1457 | O | PHE | A | 187 | 0.709 | −0.451 | 11.958 | 1.00 | 22.73 | 8 | O |
| ATOM | 1458 | CB | PHE | A | 187 | 2.987 | −0.778 | 10.182 | 1.00 | 22.97 | 6 | C |
| ATOM | 1459 | CG | PHE | A | 187 | 4.337 | −1.205 | 9.700 | 1.00 | 23.06 | 6 | C |
| ATOM | 1460 | CD1 | PHE | A | 187 | 5.018 | −0.446 | 8.750 | 1.00 | 23.35 | 6 | C |
| ATOM | 1461 | CD2 | PHE | A | 187 | 4.950 | −2.349 | 10.177 | 1.00 | 22.68 | 6 | C |
| ATOM | 1462 | CE1 | PHE | A | 187 | 6.273 | −0.831 | 8.296 | 1.00 | 23.87 | 6 | C |
| ATOM | 1463 | CE2 | PHE | A | 187 | 6.191 | −2.736 | 9.732 | 1.00 | 22.40 | 6 | C |
| ATOM | 1464 | CZ | PHE | A | 187 | 6.854 | −1.983 | 8.795 | 1.00 | 23.34 | 6 | C |
| ATOM | 1465 | N | GLN | A | 188 | −0.526 | −1.443 | 10.366 | 1.00 | 25.69 | 7 | N |
| ATOM | 1466 | CA | GLN | A | 188 | −1.751 | −0.798 | 10.830 | 1.00 | 26.36 | 6 | C |
| ATOM | 1467 | C | GLN | A | 188 | −2.288 | −1.204 | 12.181 | 1.00 | 26.42 | 6 | C |
| ATOM | 1468 | O | GLN | A | 188 | −3.126 | −0.455 | 12.710 | 1.00 | 24.16 | 8 | O |
| ATOM | 1469 | CB | GLN | A | 188 | −2.835 | −0.853 | 9.738 | 1.00 | 27.93 | 6 | C |
| ATOM | 1470 | CG | GLN | A | 188 | −2.463 | −0.104 | 8.472 | 1.00 | 29.03 | 6 | C |
| ATOM | 1471 | CD | GLN | A | 188 | −1.937 | 1.301 | 8.633 | 1.00 | 29.40 | 6 | C |
| ATOM | 1472 | OE1 | GLN | A | 188 | −2.616 | 2.218 | 9.098 | 1.00 | 29.04 | 8 | O |
| ATOM | 1473 | NE2 | GLN | A | 188 | −0.670 | 1.488 | 8.239 | 1.00 | 28.70 | 7 | N |
| ATOM | 1474 | N | ASP | A | 189 | −1.847 | −2.307 | 12.804 | 1.00 | 26.68 | 7 | N |
| ATOM | 1475 | CA | ASP | A | 189 | −2.401 | −2.601 | 14.136 | 1.00 | 28.48 | 6 | C |
| ATOM | 1476 | C | ASP | A | 189 | −1.507 | −1.954 | 15.190 | 1.00 | 27.75 | 6 | C |
| ATOM | 1477 | O | ASP | A | 189 | −1.753 | −2.029 | 16.387 | 1.00 | 26.53 | 8 | O |
| ATOM | 1478 | CB | ASP | A | 189 | −2.738 | −4.059 | 14.353 | 1.00 | 31.61 | 6 | C |
| ATOM | 1479 | CG | ASP | A | 189 | −4.020 | −4.483 | 13.650 | 1.00 | 34.21 | 6 | C |
| ATOM | 1480 | OD1 | ASP | A | 189 | −4.831 | −3.639 | 13.210 | 1.00 | 35.47 | 8 | O |
| ATOM | 1481 | OD2 | ASP | A | 189 | −4.219 | −5.713 | 13.533 | 1.00 | 37.56 | 8 | O |
| ATOM | 1482 | N | PHE | A | 190 | −0.447 | −1.264 | 14.752 | 1.00 | 27.40 | 7 | N |
| ATOM | 1483 | CA | PHE | A | 190 | −0.440 | −0.568 | 15.676 | 1.00 | 26.77 | 6 | C |
| ATOM | 1484 | C | PHE | A | 190 | −0.143 | 0.826 | 15.946 | 1.00 | 26.91 | 6 | C |
| ATOM | 1485 | O | PHE | A | 190 | −0.980 | 1.281 | 15.168 | 1.00 | 27.50 | 8 | O |
| ATOM | 1486 | CB | PHE | A | 190 | 1.821 | −0.316 | 15.075 | 1.00 | 27.37 | 6 | C |
| ATOM | 1487 | CG | PHE | A | 190 | 2.728 | −1.503 | 14.933 | 1.00 | 27.45 | 6 | C |
| ATOM | 1488 | CD1 | PHE | A | 190 | 2.693 | −2.272 | 13.790 | 1.00 | 27.73 | 6 | C |
| ATOM | 1489 | CD2 | PHE | A | 190 | 3.618 | −1.850 | 15.934 | 1.00 | 27.99 | 6 | C |
| ATOM | 1490 | CE1 | PHE | A | 190 | 3.513 | −3.369 | 13.625 | 1.00 | 27.82 | 6 | C |
| ATOM | 1491 | CE2 | PHE | A | 190 | 4.448 | −2.947 | 15.788 | 1.00 | 28.00 | 6 | C |
| ATOM | 1492 | CZ | PHE | A | 190 | 4.398 | −3.705 | 14.634 | 1.00 | 28.16 | 6 | C |
| ATOM | 1493 | N | ASN | A | 191 | 0.322 | 1.508 | 16.981 | 1.00 | 26.62 | 7 | N |
| ATOM | 1494 | CA | ASN | A | 191 | −0.125 | 2.888 | 17.211 | 1.00 | 26.91 | 6 | C |
| ATOM | 1495 | C | ASN | A | 191 | 0.894 | 3.686 | 16.379 | 1.00 | 27.03 | 6 | C |
| ATOM | 1496 | O | ASN | A | 191 | 2.008 | 3.853 | 16.884 | 1.00 | 26.68 | 8 | O |
| ATOM | 1497 | CB | ASN | A | 191 | −0.058 | 3.303 | 18.661 | 1.00 | 28.48 | 6 | C |
| ATOM | 1498 | CG | ASN | A | 191 | −0.227 | 4.797 | 18.891 | 1.00 | 29.25 | 6 | C |
| ATOM | 1499 | OD1 | ASN | A | 191 | −0.951 | 5.486 | 18.170 | 1.00 | 30.13 | 8 | O |
| ATOM | 1500 | ND2 | ASN | A | 191 | 0.475 | 5.269 | 19.921 | 1.00 | 28.77 | 7 | N |
| ATOM | 1501 | N | ILE | A | 192 | 0.536 | 4.063 | 15.158 | 1.00 | 26.25 | 7 | N |
| ATOM | 1502 | CA | ILE | A | 192 | 1.505 | 4.762 | 14.328 | 1.00 | 25.77 | 6 | C |
| ATOM | 1503 | C | ILE | A | 192 | 1.613 | 6.232 | 14.688 | 1.00 | 25.97 | 6 | C |
| ATOM | 1504 | O | ILE | A | 192 | 0.632 | 6.958 | 14.800 | 1.00 | 24.17 | 8 | O |
| ATOM | 1505 | CB | ILE | A | 192 | 1.232 | 4.612 | 12.823 | 1.00 | 26.16 | 6 | C |
| ATOM | 1506 | CG1 | ILE | A | 192 | 1.110 | 3.132 | 12.449 | 1.00 | 26.35 | 6 | C |
| ATOM | 1507 | CG2 | ILE | A | 192 | 2.356 | 5.268 | 12.033 | 1.00 | 26.27 | 6 | C |
| ATOM | 1508 | CD1 | ILE | A | 192 | 0.466 | 2.909 | 11.090 | 1.00 | 26.82 | 6 | C |
| ATOM | 1509 | N | ILE | A | 193 | 2.872 | 6.616 | 14.891 | 1.00 | 25.38 | 7 | N |
| ATOM | 1510 | CA | ILE | A | 193 | 3.237 | 7.994 | 15.213 | 1.00 | 26.05 | 6 | C |
| ATOM | 1511 | C | ILE | A | 193 | 3.916 | 8.526 | 13.953 | 1.00 | 25.47 | 6 | C |
| ATOM | 1512 | O | ILE | A | 193 | 5.080 | 8.278 | 13.645 | 1.00 | 24.43 | 8 | O |
| ATOM | 1513 | CB | ILE | A | 193 | 4.132 | 8.070 | 16.454 | 1.00 | 26.39 | 6 | C |
| ATOM | 1514 | CG1 | ILE | A | 193 | 3.420 | 7.423 | 17.651 | 1.00 | 27.17 | 6 | C |
| ATOM | 1515 | CG2 | ILE | A | 193 | 4.508 | 9.509 | 16.751 | 1.00 | 26.91 | 6 | C |
| ATOM | 1516 | CD1 | ILE | A | 193 | 4.388 | 6.855 | 18.664 | 1.00 | 27.04 | 6 | C |
| ATOM | 1517 | N | GLU | A | 194 | 3.106 | 9.248 | 13.165 | 1.00 | 25.39 | 7 | N |
| ATOM | 1518 | CA | GLU | A | 194 | 3.568 | 9.817 | 11.910 | 1.00 | 24.62 | 6 | C |
| ATOM | 1519 | C | GLU | A | 194 | 4.497 | 10.998 | 12.162 | 1.00 | 24.98 | 6 | C |
| ATOM | 1520 | O | GLU | A | 194 | 4.125 | 11.957 | 12.831 | 1.00 | 25.67 | 8 | O |
| ATOM | 1521 | CB | GLU | A | 194 | 2.384 | 10.238 | 11.038 | 1.00 | 24.73 | 6 | C |
| ATOM | 1522 | CG | GLU | A | 194 | 1.519 | 9.058 | 10.538 | 0.00 | 20.00 | 6 | C |
| ATOM | 1523 | CD | GLU | A | 194 | 0.344 | 9.504 | 9.691 | 0.00 | 20.00 | 6 | C |
| ATOM | 1524 | OE1 | GLU | A | 194 | 0.070 | 10.721 | 9.646 | 0.00 | 20.00 | 8 | O |
| ATOM | 1525 | OE2 | GLU | A | 194 | −0.303 | 8.634 | 9.070 | 0.00 | 20.00 | 8 | O |
| ATOM | 1526 | N | ILE | A | 195 | 5.707 | 10.918 | 11.628 | 1.00 | 24.28 | 7 | N |
| ATOM | 1527 | CA | ILE | A | 195 | 6.691 | 11.991 | 11.796 | 1.00 | 24.01 | 6 | C |
| ATOM | 1528 | C | ILE | A | 195 | 6.452 | 13.048 | 10.727 | 1.00 | 25.07 | 6 | C |
| ATOM | 1529 | O | ILE | A | 195 | 6.336 | 12.690 | 9.558 | 1.00 | 23.72 | 8 | O |
| ATOM | 1530 | CB | ILE | A | 195 | 8.100 | 11.379 | 11.656 | 1.00 | 23.50 | 6 | C |
| ATOM | 1531 | CG1 | ILE | A | 195 | 8.331 | 10.272 | 12.685 | 1.00 | 22.60 | 6 | C |
| ATOM | 1532 | CG2 | ILE | A | 195 | 9.179 | 12.447 | 11.771 | 1.00 | 23.57 | 6 | C |
| ATOM | 1533 | CD1 | ILE | A | 195 | 8.133 | 10.627 | 14.134 | 1.00 | 22.07 | 6 | C |
| ATOM | 1534 | N | PRO | A | 196 | 6.361 | 14.316 | 11.090 | 1.00 | 25.77 | 7 | N |
| ATOM | 1535 | CA | PRO | A | 196 | 6.147 | 15.402 | 10.137 | 1.00 | 26.34 | 6 | C |
| ATOM | 1536 | C | PRO | A | 196 | 7.272 | 15.479 | 9.118 | 1.00 | 26.73 | 6 | C |
| ATOM | 1537 | O | PRO | A | 196 | 8.407 | 15.175 | 9.487 | 1.00 | 26.66 | 8 | O |
| ATOM | 1538 | CB | PRO | A | 196 | 6.092 | 16.653 | 10.996 | 1.00 | 26.61 | 6 | C |
| ATOM | 1539 | CG | PRO | A | 196 | 5.912 | 16.203 | 12.403 | 1.00 | 26.62 | 6 | C |
| ATOM | 1540 | CD | PRO | A | 196 | 6.488 | 14.805 | 12.475 | 1.00 | 26.63 | 6 | C |
| ATOM | 1541 | N | GLU | A | 197 | 7.008 | 15.896 | 7.878 | 1.00 | 27.22 | 7 | N |
| ATOM | 1542 | CA | GLU | A | 197 | 8.042 | 15.940 | 6.853 | 1.00 | 28.33 | 6 | C |
| ATOM | 1543 | C | GLU | A | 197 | 9.299 | 16.705 | 7.179 | 1.00 | 27.30 | 6 | C |
| ATOM | 1544 | O | GLU | A | 197 | 10.386 | 16.191 | 6.889 | 1.00 | 26.37 | 8 | O |
| ATOM | 1545 | CB | GLU | A | 197 | 7.469 | 16.400 | 5.515 | 1.00 | 30.07 | 6 | C |
| ATOM | 1546 | CG | GLU | A | 197 | 6.964 | 17.827 | 5.443 | 1.00 | 32.37 | 6 | C |
| ATOM | 1547 | CD | GLU | A | 197 | 6.620 | 18.137 | 3.983 | 1.00 | 33.19 | 6 | C |
| ATOM | 1548 | OE1 | GLU | A | 197 | 7.575 | 18.431 | 3.225 | 1.00 | 33.05 | 8 | O |
| ATOM | 1549 | OE2 | GLU | A | 197 | 5.428 | 18.071 | 3.652 | 1.00 | 33.61 | 8 | O |
| ATOM | 1550 | N | GLU | A | 198 | 9.232 | 17.881 | 7.776 | 1.00 | 27.01 | 7 | N |
| ATOM | 1551 | CA | GLU | A | 198 | 10.431 | 18.642 | 8.118 | 1.00 | 27.58 | 6 | C |
| ATOM | 1552 | C | GLU | A | 198 | 11.324 | 17.920 | 9.119 | 1.00 | 26.44 | 6 | C |
| ATOM | 1553 | O | GLU | A | 198 | 12.533 | 18.162 | 9.196 | 1.00 | 27.72 | 8 | O |
| ATOM | 1554 | CB | GLU | A | 198 | 9.930 | 19.958 | 8.757 | 1.00 | 29.42 | 6 | C |
| ATOM | 1555 | CG | GLU | A | 198 | 9.165 | 19.601 | 10.033 | 1.00 | 31.31 | 6 | C |
| ATOM | 1556 | CD | GLU | A | 198 | 7.755 | 20.138 | 10.037 | 1.00 | 31.76 | 6 | C |
| ATOM | 1557 | OE1 | GLU | A | 198 | 7.084 | 20.077 | 8.984 | 1.00 | 33.08 | 8 | O |
| ATOM | 1558 | OE2 | GLU | A | 198 | 7.409 | 20.600 | 11.145 | 1.00 | 32.34 | 8 | O |
| ATOM | 1559 | N | GLU | A | 199 | 10.734 | 17.032 | 9.911 | 1.00 | 23.98 | 7 | N |
| ATOM | 1560 | CA | GLU | A | 199 | 11.406 | 16.270 | 10.938 | 1.00 | 22.84 | 6 | C |
| ATOM | 1561 | C | GLU | A | 199 | 11.629 | 14.807 | 10.590 | 1.00 | 21.79 | 6 | C |
| ATOM | 1562 | O | GLU | A | 199 | 11.897 | 14.011 | 11.502 | 1.00 | 21.82 | 8 | O |
| ATOM | 1563 | CB | GLU | A | 199 | 10.553 | 16.324 | 12.223 | 1.00 | 21.92 | 6 | C |
| ATOM | 1564 | CG | GLU | A | 199 | 10.375 | 17.757 | 12.720 | 1.00 | 22.63 | 6 | C |
| ATOM | 1565 | CD | GLU | A | 199 | 10.059 | 17.854 | 14.186 | 1.00 | 23.27 | 6 | C |
| ATOM | 1566 | OE1 | GLU | A | 199 | 9.866 | 16.822 | 14.862 | 1.00 | 23.08 | 8 | O |
| ATOM | 1567 | OE2 | GLU | A | 199 | 9.992 | 18.980 | 14.723 | 1.00 | 24.02 | 8 | O |
| ATOM | 1568 | N | SER | A | 200 | 11.536 | 14.430 | 9.325 | 1.00 | 21.70 | 7 | N |
| ATOM | 1569 | CA | SER | A | 200 | 11.718 | 13.055 | 8.879 | 1.00 | 21.83 | 6 | C |
| ATOM | 1570 | C | SER | A | 200 | 12.903 | 12.324 | 9.487 | 1.00 | 20.73 | 6 | C |
| ATOM | 1571 | O | SER | A | 200 | 12.785 | 11.132 | 9.792 | 1.00 | 18.50 | 8 | O |
| ATOM | 1572 | CB | SER | A | 200 | 11.869 | 12.995 | 7.347 | 1.00 | 22.88 | 6 | C |
| ATOM | 1573 | OG | SER | A | 200 | 13.051 | 13.701 | 6.968 | 1.00 | 27.44 | 8 | O |
| ATOM | 1574 | N | TYR | A | 201 | 14.055 | 12.977 | 9.646 | 1.00 | 20.73 | 7 | N |
| ATOM | 1575 | CA | TYR | A | 201 | 15.239 | 12.357 | 10.234 | 1.00 | 21.72 | 6 | C |
| ATOM | 1576 | C | TYR | A | 201 | 14.917 | 11.753 | 11.589 | 1.00 | 20.66 | 6 | C |
| ATOM | 1577 | O | TYR | A | 201 | 15.415 | 10.675 | 11.914 | 1.00 | 20.15 | 8 | O |
| ATOM | 1578 | CB | TYR | A | 201 | 16.369 | 13.371 | 10.430 | 1.00 | 22.13 | 6 | C |
| ATOM | 1579 | CG | TYR | A | 201 | 17.693 | 12.886 | 10.969 | 1.00 | 22.75 | 6 | C |
| ATOM | 1580 | CD1 | TYR | A | 201 | 18.475 | 11.965 | 10.270 | 1.00 | 23.12 | 6 | C |
| ATOM | 1581 | CD2 | TYR | A | 201 | 18.181 | 13.354 | 12.185 | 1.00 | 22.51 | 6 | C |
| ATOM | 1582 | CE1 | TYR | A | 201 | 19.691 | 11.532 | 10.773 | 1.00 | 23.47 | 6 | C |
| ATOM | 1583 | CE2 | TYR | A | 201 | 19.392 | 12.924 | 12.688 | 1.00 | 22.81 | 6 | C |
| ATOM | 1584 | CZ | TYR | A | 201 | 20.149 | 12.017 | 11.985 | 1.00 | 23.29 | 6 | C |
| ATOM | 1585 | OH | TYR | A | 201 | 21.363 | 11.580 | 12.471 | 1.00 | 21.41 | 8 | O |
| ATOM | 1586 | N | ALA | A | 202 | 14.071 | 12.404 | 12.385 | 1.00 | 19.51 | 7 | N |
| ATOM | 1587 | CA | ALA | A | 202 | 13.695 | 11.933 | 13.704 | 1.00 | 18.95 | 6 | C |
| ATOM | 1588 | C | ALA | A | 202 | 12.847 | 10.687 | 13.764 | 1.00 | 18.73 | 6 | C |
| ATOM | 1589 | O | ALA | A | 202 | 12.430 | 10.278 | 14.855 | 1.00 | 19.37 | 8 | O |
| ATOM | 1590 | CB | ALA | A | 202 | 13.070 | 13.058 | 14.527 | 1.00 | 20.14 | 6 | C |
| ATOM | 1591 | N | ALA | A | 203 | 12.594 | 9.990 | 12.654 | 1.00 | 19.16 | 7 | N |
| ATOM | 1592 | CA | ALA | A | 203 | 11.864 | 8.734 | 12.665 | 1.00 | 19.15 | 6 | C |
| ATOM | 1593 | C | ALA | A | 203 | 12.836 | 7.661 | 13.185 | 1.00 | 19.93 | 6 | C |
| ATOM | 1594 | O | ALA | A | 203 | 12.425 | 6.570 | 13.592 | 1.00 | 19.88 | 8 | O |
| ATOM | 1595 | CB | ALA | A | 203 | 11.365 | 8.307 | 11.303 | 1.00 | 19.46 | 6 | C |
| ATOM | 1596 | N | ASN | A | 204 | 14.138 | 7.991 | 13.127 | 1.00 | 18.23 | 7 | N |
| ATOM | 1597 | CA | ASN | A | 204 | 15.141 | 7.047 | 13.640 | 1.00 | 18.46 | 6 | C |
| ATOM | 1598 | C | ASN | A | 204 | 15.439 | 7.447 | 15.090 | 1.00 | 18.04 | 6 | C |
| ATOM | 1599 | O | ASN | A | 204 | 16.051 | 8.486 | 15.346 | 1.00 | 16.74 | 8 | O |
| ATOM | 1600 | CB | ASN | A | 204 | 16.378 | 7.089 | 12.759 | 1.00 | 18.18 | 6 | C |
| ATOM | 1601 | CG | ASN | A | 204 | 17.429 | 6.060 | 13.089 | 1.00 | 20.67 | 6 | C |
| ATOM | 1602 | OD1 | ASN | A | 204 | 17.833 | 5.245 | 12.250 | 1.00 | 22.55 | 8 | O |
| ATOM | 1603 | ND2 | ASN | A | 204 | 17.914 | 6.052 | 14.311 | 1.00 | 20.41 | 7 | N |
| ATOM | 1604 | N | CYH | A | 205 | 14.952 | 6.662 | 16.044 | 1.00 | 18.20 | 7 | N |
| ATOM | 1605 | CA | CYH | A | 205 | 15.111 | 6.860 | 17.465 | 1.00 | 17.46 | 6 | C |
| ATOM | 1606 | C | CYH | A | 205 | 15.321 | 5.491 | 18.116 | 1.00 | 17.44 | 6 | C |
| ATOM | 1607 | O | CYH | A | 205 | 15.029 | 4.489 | 17.469 | 1.00 | 18.02 | 8 | O |
| ATOM | 1608 | CB | CYH | A | 205 | 13.899 | 7.489 | 18.162 | 1.00 | 17.85 | 6 | C |
| ATOM | 1609 | SG | CYH | A | 205 | 13.520 | 9.185 | 17.634 | 1.00 | 17.21 | 16 | S |
| ATOM | 1610 | N | ILE | A | 206 | 15.846 | 5.459 | 19.345 | 1.00 | 17.51 | 7 | N |
| ATOM | 1611 | CA | ILE | A | 206 | 16.007 | 4.170 | 20.011 | 1.00 | 16.74 | 6 | C |
| ATOM | 1612 | C | ILE | A | 206 | 15.418 | 4.115 | 21.416 | 1.00 | 17.76 | 6 | C |
| ATOM | 1613 | O | ILE | A | 206 | 15.635 | 4.957 | 22.283 | 1.00 | 18.75 | 8 | O |
| ATOM | 1614 | CB | ILE | A | 206 | 17.473 | 3.701 | 20.152 | 1.00 | 16.12 | 6 | C |
| ATOM | 1615 | CG1 | ILE | A | 206 | 18.349 | 4.796 | 20.762 | 1.00 | 14.91 | 6 | C |
| ATOM | 1616 | CG2 | ILE | A | 206 | 17.997 | 3.224 | 18.809 | 1.00 | 14.96 | 6 | C |
| ATOM | 1617 | CD1 | ILE | A | 206 | 19.807 | 4.371 | 20.956 | 1.00 | 15.94 | 6 | C |
| ATOM | 1618 | N | TRP | A | 207 | 14.634 | 3.065 | 21.650 | 1.00 | 18.53 | 7 | N |
| ATOM | 1619 | CA | TRP | A | 207 | 14.056 | 2.794 | 22.953 | 1.00 | 18.99 | 6 | C |
| ATOM | 1620 | C | TRP | A | 207 | 15.157 | 1.993 | 23.669 | 1.00 | 18.45 | 6 | C |
| ATOM | 1621 | O | TRP | A | 207 | 15.746 | 1.093 | 23.068 | 1.00 | 17.12 | 8 | O |
| ATOM | 1622 | CB | TRP | A | 207 | 12.806 | 1.927 | 22.883 | 1.00 | 18.96 | 6 | C |
| ATOM | 1623 | CG | TRP | A | 207 | 12.266 | 1.462 | 24.200 | 1.00 | 19.37 | 6 | C |
| ATOM | 1624 | CD1 | TRP | A | 207 | 11.309 | 2.103 | 24.944 | 1.00 | 19.32 | 6 | C |
| ATOM | 1625 | CD2 | TRP | A | 207 | 12.633 | 0.294 | 24.927 | 1.00 | 19.14 | 6 | C |
| ATOM | 1626 | NE1 | TRP | A | 207 | 11.057 | 1.390 | 26.098 | 1.00 | 20.24 | 7 | N |
| ATOM | 1627 | CE2 | TRP | A | 207 | 11.863 | 0.272 | 26.104 | 1.00 | 20.17 | 6 | C |
| ATOM | 1628 | CE3 | TRP | A | 207 | 13.542 | −0.750 | 24.700 | 1.00 | 19.82 | 6 | C |
| ATOM | 1629 | CZ2 | TRP | A | 207 | 11.968 | −0.745 | 27.053 | 1.00 | 20.63 | 6 | C |
| ATOM | 1630 | CZ3 | TRP | A | 207 | 13.649 | −1.760 | 25.642 | 1.00 | 19.84 | 6 | C |
| ATOM | 1631 | CH2 | TRP | A | 207 | 12.864 | −1.755 | 26.810 | 1.00 | 20.72 | 6 | C |
| ATOM | 1632 | N | VAL | A | 208 | 15.480 | 2.363 | 24.896 | 1.00 | 19.46 | 7 | N |
| ATOM | 1633 | CA | VAL | A | 208 | 16.457 | 1.665 | 25.714 | 1.00 | 18.65 | 6 | C |
| ATOM | 1634 | C | VAL | A | 208 | 15.919 | 1.669 | 27.152 | 1.00 | 18.51 | 6 | C |
| ATOM | 1635 | O | VAL | A | 208 | 16.056 | 2.672 | 27.866 | 1.00 | 18.53 | 8 | O |
| ATOM | 1636 | CB | VAL | A | 208 | 17.894 | 2.221 | 25.735 | 1.00 | 18.71 | 6 | C |
| ATOM | 1637 | CG1 | VAL | A | 208 | 18.841 | 1.287 | 26.497 | 1.00 | 17.63 | 6 | C |
| ATOM | 1638 | CG2 | VAL | A | 208 | 18.495 | 2.479 | 24.360 | 1.00 | 19.19 | 6 | C |
| ATOM | 1639 | N | ASN | A | 209 | 15.288 | 0.598 | 27.620 | 1.00 | 18.42 | 7 | N |
| ATOM | 1640 | CA | ASN | A | 209 | 14.818 | 0.540 | 28.999 | 1.00 | 19.92 | 6 | C |
| ATOM | 1641 | C | ASN | A | 209 | 13.987 | 1.693 | 29.517 | 1.00 | 20.22 | 6 | C |
| ATOM | 1642 | O | ASN | A | 209 | 14.147 | 2.271 | 30.606 | 1.00 | 19.91 | 8 | O |
| ATOM | 1643 | CB | ASN | A | 209 | 16.088 | 0.372 | 29.874 | 1.00 | 20.08 | 6 | C |
| ATOM | 1644 | CG | ASN | A | 209 | 16.769 | −0.963 | 29.641 | 1.00 | 20.53 | 6 | C |
| ATOM | 1645 | OD1 | ASN | A | 209 | 16.236 | −1.848 | 28.967 | 1.00 | 21.40 | 8 | O |
| ATOM | 1646 | ND2 | ASN | A | 209 | 17.972 | −1.151 | 30.195 | 1.00 | 19.40 | 7 | N |
| ATOM | 1647 | N | GLU | A | 210 | 12.999 | 2.078 | 28.731 | 1.00 | 21.23 | 7 | N |
| ATOM | 1648 | CA | GLU | A | 210 | 12.019 | 3.119 | 28.985 | 1.00 | 21.73 | 6 | C. |
| ATOM | 1649 | C | GLU | A | 210 | 12.514 | 4.529 | 28.735 | 1.00 | 21.32 | 6 | C |
| ATOM | 1650 | O | GLU | A | 210 | 11.766 | 5.499 | 28.905 | 1.00 | 21.06 | 8 | O |
| ATOM | 1651 | CB | GLU | A | 210 | 11.295 | 2.948 | 30.310 | 1.00 | 23.97 | 6 | C |
| ATOM | 1652 | CG | GLU | A | 210 | 10.520 | 1.663 | 30.487 | 1.00 | 26.15 | 6 | C |
| ATOM | 1653 | CD | GLU | A | 210 | 9.616 | 1.142 | 29.398 | 1.00 | 28.00 | 6 | C |
| ATOM | 1654 | OE1 | GLU | A | 210 | 9.199 | 1.860 | 28.471 | 1.00 | 26.92 | 8 | O |
| ATOM | 1655 | OE2 | GLU | A | 210 | 9.302 | −0.080 | 29.477 | 1.00 | 29.47 | 8 | O |
| ATOM | 1656 | N | ARG | A | 211 | 13.760 | 4.676 | 28.307 | 1.00 | 20.42 | 7 | N |
| ATOM | 1657 | CA | ARG | A | 211 | 14.353 | 5.932 | 27.916 | 1.00 | 20.26 | 6 | C |
| ATOM | 1658 | C | ARG | A | 211 | 14.236 | 5.907 | 26.369 | 1.00 | 19.19 | 6 | C |
| ATOM | 1659 | O | ARG | A | 211 | 14.214 | 4.803 | 25.792 | 1.00 | 19.00 | 8 | O |
| ATOM | 1660 | CB | ARG | A | 211 | 15.844 | 6.084 | 28.198 | 1.00 | 21.37 | 6 | C |
| ATOM | 1661 | CG | ARG | A | 211 | 16.382 | 5.228 | 29.329 | 1.00 | 25.62 | 6 | C |
| ATOM | 1662 | CD | ARG | A | 211 | 16.317 | 5.857 | 30.661 | 1.00 | 24.95 | 6 | C |
| ATOM | 1663 | NE | ARG | A | 211 | 16.816 | 5.286 | 31.850 | 1.00 | 24.48 | 7 | N |
| ATOM | 1664 | CZ | ARG | A | 211 | 16.847 | 4.305 | 32.700 | 1.00 | 24.23 | 6 | C |
| ATOM | 1665 | NH1 | ARG | A | 211 | 17.629 | 4.406 | 33.786 | 1.00 | 23.57 | 7 | N |
| ATOM | 1666 | NH2 | ARG | A | 211 | 16.142 | 3.180 | 32.551 | 1.00 | 23.60 | 7 | N |
| ATOM | 1667 | N | VAL | A | 212 | 14.140 | 7.052 | 25.733 | 1.00 | 18.18 | 7 | N |
| ATOM | 1668 | CA | VAL | A | 212 | 14.096 | 7.057 | 24.262 | 1.00 | 17.19 | 6 | C |
| ATOM | 1669 | C | VAL | A | 212 | 15.160 | .8.084 | 23.862 | 1.00 | 18.50 | 6 | C |
| ATOM | 1670 | O | VAL | A | 212 | 15.053 | 9.264 | 24.223 | 1.00 | 20.05 | 8 | O |
| ATOM | 1671 | CB | VAL | A | 212 | 12.763 | 7.306 | 23.578 | 1.00 | 18.07 | 6 | C |
| ATOM | 1672 | CG1 | VAL | A | 212 | 12.995 | 7.592 | 22.083 | 1.00 | 16.31 | 6 | C |
| ATOM | 1673 | CG2 | VAL | A | 212 | 11.824 | 6.104 | 23.634 | 1.00 | 16.24 | 6 | C |
| ATOM | 1674 | N | ILE | A | 213 | 16.187 | 7.634 | 23.162 | 1.00 | 17.23 | 7 | N |
| ATOM | 1675 | CA | ILE | A | 213 | 17.269 | 8.524 | 22.726 | 1.00 | 17.43 | 6 | C |
| ATOM | 1676 | C | ILE | A | 213 | 16.946 | 9.001 | 21.316 | 1.00 | 16.63 | 6 | C |
| ATOM | 1677 | O | ILE | A | 213 | 16.682 | 8.155 | 20.452 | 1.00 | 16.00 | 8 | O |
| ATOM | 1678 | CB | ILE | A | 213 | 18.639 | 7.842 | 22.823 | 1.00 | 18.86 | 6 | C |
| ATOM | 1679 | CG1 | ILE | A | 213 | 18.978 | 7.581 | 24.306 | 1.00 | 20.36 | 6 | C |
| ATOM | 1680 | CG2 | ILE | A | 213 | 19.723 | 8.695 | 22.184 | 1.00 | 19.06 | 6 | C |
| ATOM | 1681 | CD1 | ILE | A | 213 | 20.196 | 6.711 | 24.545 | 1.00 | 20.60 | 6 | C |
| ATOM | 1682 | N | MET | A | 214 | 16.943 | 10.303 | 21.071 | 1.00 | 16.36 | 7 | N |
| ATOM | 1683 | CA | MET | A | 214 | 16.594 | 10.806 | 19.740 | 1.00 | 16.59 | 6 | C |
| ATOM | 1684 | C | MET | A | 214 | 17.494 | 11.968 | 19.361 | 1.00 | 18.17 | 6 | C |
| ATOM | 1685 | O | MET | A | 214 | 18.125 | 12.563 | 20.254 | 1.00 | 18.22 | 8 | O |
| ATOM | 1686 | CB | MET | A | 214 | 15.114 | 11.287 | 19.767 | 1.00 | 17.07 | 6 | C |
| ATOM | 1687 | CG | MET | A | 214 | 14.976 | 12.564 | 20.594 | 1.00 | 16.98 | 6 | C |
| ATOM | 1688 | SD | MET | A | 214 | 13.318 | 13.301 | 20.624 | 1.00 | 16.73 | 16 | S |
| ATOM | 1689 | CE | MET | A | 214 | 13.119 | 13.649 | 18.884 | 1.00 | 15.25 | 6 | C |
| ATOM | 1690 | N | PRO | A | 215 | 17.560 | 12.283 | 18.080 | 1.00 | 18.01 | 7 | N |
| ATOM | 1691 | CA | PRO | A | 215 | 18.353 | 13.409 | 17.601 | 1.00 | 18.79 | 6 | C |
| ATOM | 1692 | C | PRO | A | 215 | 17.789 | 14.714 | 18.155 | 1.00 | 19.48 | 6 | C |
| ATOM | 1693 | O | PRO | A | 215 | 16.565 | 14.888 | 18.294 | 1.00 | 19.18 | 8 | O |
| ATOM | 1694 | CB | PRO | A | 215 | 18.202 | 13.366 | 16.091 | 1.00 | 18.85 | 6 | C |
| ATOM | 1695 | CG | PRO | A | 215 | 16.950 | 12.595 | 15.835 | 1.00 | 19.64 | 6 | C |
| ATOM | 1696 | CD | PRO | A | 215 | 16.843 | 11.607 | 16.973 | 1.00 | 18.95 | 6 | C |
| ATOM | 1697 | N | ALA | A | 216 | 18.690 | 15.642 | 18.482 | 1.00 | 19.80 | 7 | N |
| ATOM | 1698 | CA | ALA | A | 216 | 18.273 | 16.939 | 19.013 | 1.00 | 20.58 | 6 | C |
| ATOM | 1699 | C | ALA | A | 216 | 17.847 | 17.841 | 17.845 | 1.00 | 20.94 | 6 | C |
| ATOM | 1700 | O | ALA | A | 216 | 18.416 | 17.694 | 16.769 | 1.00 | 20.67 | 8 | O |
| ATOM | 1701 | CB | ALA | A | 216 | 19.416 | 17.601 | 19.761 | 1.00 | 20.83 | 6 | C |
| ATOM | 1702 | N | GLY | A | 217 | 16.880 | 18.724 | 18.074 | 1.00 | 21.90 | 7 | N |
| ATOM | 1703 | CA | GLY | A | 217 | 16.418 | 19.627 | 17.031 | 1.00 | 20.96 | 6 | C |
| ATOM | 1704 | C | GLY | A | 217 | 15.142 | 19.245 | 16.328 | 1.00 | 22.06 | 6 | C |
| ATOM | 1705 | O | GLY | A | 217 | 14.815 | 19.759 | 15.242 | 1.00 | 22.32 | 8 | O |
| ATOM | 1706 | N | TYR | A | 218 | 14.355 | 18.317 | 16.863 | 1.00 | 20.52 | 7 | N |
| ATOM | 1707 | CA | TYR | A | 218 | 13.095 | 17.823 | 16.277 | 1.00 | 20.87 | 6 | C |
| ATOM | 1708 | C | TYR | A | 218 | 12.042 | 17.893 | 17.368 | 1.00 | 20.81 | 6 | C |
| ATOM | 1709 | O | TYR | A | 218 | 11.639 | 16.915 | 17.994 | 1.00 | 20.94 | 8 | O |
| ATOM | 1710 | CB | TYR | A | 218 | 13.338 | 16.412 | 15.689 | 1.00 | 20.31 | 6 | C |
| ATOM | 1711 | CG | TYR | A | 218 | 14.457 | 16.446 | 14.649 | 1.00 | 20.88 | 6 | C |
| ATOM | 1712 | CD1 | TYR | A | 218 | 15.754 | 16.045 | 14.963 | 1.00 | 21.21 | 6 | C |
| ATOM | 1713 | CD2 | TYR | A | 218 | 14.215 | 16.908 | 13.359 | 1.00 | 20.98 | 6 | C |
| ATOM | 1714 | CE1 | TYR | A | 218 | 16.756 | 16.098 | 14.010 | 1.00 | 20.95 | 6 | C |
| ATOM | 1715 | CE2 | TYR | A | 218 | 15.214 | 16.960 | 12.412 | 1.00 | 21.20 | 6 | C |
| ATOM | 1716 | CZ | TYR | A | 218 | 16.491 | 16.550 | 12.736 | 1.00 | 20.49 | 6 | C |
| ATOM | 1717 | OH | TYR | A | 218 | 17.477 | 16.607 | 11.786 | 1.00 | 19.44 | 8 | O |
| ATOM | 1718 | N | PRO | A | 219 | 11.565 | 19.114 | 17.669 | 1.00 | 21.54 | 7 | N |
| ATOM | 1719 | CA | PRO | A | 219 | 10.608 | 19.342 | 18.729 | 1.00 | 21.50 | 6 | C |
| ATOM | 1720 | C | PRO | A | 219 | 9.243 | 18.706 | 18.643 | 1.00 | 22.06 | 6 | C |
| ATOM | 1721 | O | PRO | A | 219 | 8.710 | 18.332 | 19.704 | 1.00 | 21.09 | 8 | O |
| ATOM | 1722 | CB | PRO | A | 219 | 10.481 | 20.860 | 18.837 | 1.00 | 20.28 | 6 | C |
| ATOM | 1723 | CG | PRO | A | 219 | 11.169 | 21.451 | 17.672 | 1.00 | 21.21 | 6 | C |
| ATOM | 1724 | CD | PRO | A | 219 | 11.963 | 20.374 | 17.003 | 1.00 | 21.32 | 6 | C |
| ATOM | 1725 | N | ARG | A | 220 | 8.664 | 18.575 | 17.454 | 1.00 | 22.05 | 7 | N |
| ATOM | 1726 | CA | ARG | A | 220 | 7.330 | 17.980 | 17.368 | 1.00 | 23.17 | 6 | C |
| ATOM | 1727 | C | ARG | A | 220 | 7.364 | 16.496 | 17.725 | 1.00 | 22.85 | 6 | C |
| ATOM | 1728 | O | ARG | A | 220 | 6.513 | 15.958 | 18.420 | 1.00 | 21.85 | 8 | O |
| ATOM | 1729 | CB | ARG | A | 220 | 6.707 | 18.197 | 15.991 | 1.00 | 23.83 | 6 | C |
| ATOM | 1730 | CG | ARG | A | 220 | 6.322 | 19.653 | 15.725 | 1.00 | 25.67 | 6 | C |
| ATOM | 1731 | CD | ARG | A | 220 | 5.798 | 19.825 | 14.295 | 1.00 | 26.64 | 6 | C |
| ATOM | 1732 | NE | ARG | A | 220 | 4.519 | 19.125 | 14.167 | 1.00 | 28.69 | 7 | N |
| ATOM | 1733 | CZ | ARG | A | 220 | 3.815 | 18.968 | 13.052 | 1.00 | 29.17 | 6 | C |
| ATOM | 1734 | NH1 | ARG | A | 220 | 4.233 | 19.461 | 11.892 | 1.00 | 28.92 | 7 | N |
| ATOM | 1735 | NH2 | ARG | A | 220 | 2.667 | 18.301 | 13.101 | 1.00 | 29.71 | 7 | N |
| ATOM | 1736 | N | THR | A | 221 | 8.394 | 15.835 | 17.202 | 1.00 | 21.77 | 7 | N |
| ATOM | 1737 | CA | THR | A | 221 | 8.598 | 14.412 | 17.439 | 1.00 | 21.40 | 6 | C |
| ATOM | 1738 | C | THR | A | 221 | 8.927 | 14.161 | 18.901 | 1.00 | 21.15 | 6 | C |
| ATOM | 1739 | O | THR | A | 221 | 8.436 | 13.227 | 19.535 | 1.00 | 19.07 | 8 | O |
| ATOM | 1740 | CB | THR | A | 221 | 9.718 | 13.870 | 16.530 | 1.00 | 21.00 | 6 | C |
| ATOM | 1741 | OG1 | THR | A | 221 | 9.312 | 13.975 | 15.158 | 1.00 | 21.10 | 8 | O |
| ATOM | 1742 | CG2 | THR | A | 221 | 9.999 | 12.416 | 16.874 | 1.00 | 20.88 | 6 | C |
| ATOM | 1743 | N | ARG | A | 222 | 9.749 | 15.036 | 19.487 | 1.00 | 22.16 | 7 | N |
| ATOM | 1744 | CA | ARG | A | 222 | 10.101 | 14.919 | 20.901 | 1.00 | 24.31 | 6 | C |
| ATOM | 1745 | C | ARG | A | 222 | 8.853 | 14.968 | 21.768 | 1.00 | 24.22 | 6 | C |
| ATOM | 1746 | O | ARG | A | 222 | 8.722 | 14.232 | 22.750 | 1.00 | 22.96 | 8 | O |
| ATOM | 1747 | CB | ARG | A | 222 | 11.091 | 16.029 | 21.259 | 1.00 | 25.49 | 6 | C |
| ATOM | 1748 | CG | ARG | A | 222 | 11.526 | 16.046 | 22.719 | 1.00 | 28.91 | 6 | C |
| ATOM | 1749 | CD | ARG | A | 222 | 10.666 | 17.066 | 23.451 | 1.00 | 31.88 | 6 | C |
| ATOM | 1750 | NE | ARG | A | 222 | 10.430 | 16.764 | 24.840 | 1.00 | 34.87 | 7 | N |
| ATOM | 1751 | CZ | ARG | A | 222 | 11.322 | 16.630 | 25.807 | 1.00 | 36.10 | 6 | C |
| ATOM | 1752 | NH1 | ARG | A | 222 | 12.617 | 16.770 | 25.557 | 1.00 | 37.50 | 7 | N |
| ATOM | 1753 | NH2 | ARG | A | 222 | 10.933 | 16.350 | 27.046 | 1.00 | 37.08 | 7 | N |
| ATOM | 1754 | N | GLU | A | 223 | 7.892 | 15.833 | 21.427 | 1.00 | 25.65 | 7 | N |
| ATOM | 1755 | CA | GLU | A | 223 | 6.660 | 15.911 | 22.223 | 1.00 | 26.66 | 6 | C |
| ATOM | 1756 | C | GLU | A | 223 | 5.750 | 14.718 | 21.982 | 1.00 | 26.96 | 6 | C |
| ATOM | 1757 | O | GLU | A | 223 | 5.073 | 14.251 | 22.915 | 1.00 | 26.89 | 8 | O |
| ATOM | 1758 | CB | GLU | A | 223 | 5.954 | 17.247 | 21.971 | 1.00 | 29.05 | 6 | C |
| ATOM | 1759 | CG | GLU | A | 223 | 4.721 | 17.453 | 22.834 | 1.00 | 32.22 | 6 | C |
| ATOM | 1760 | CD | GLU | A | 223 | 4.989 | 7.529 | 24.321 | 1.00 | 34.06 | 6 | C |
| ATOM | 1761 | OE1 | GLU | A | 223 | 6.157 | 17.640 | 24.751 | 1.00 | 35.48 | 8 | O |
| ATOM | 1762 | OE2 | GLU | A | 223 | 4.002 | 17.478 | 25.094 | 1.00 | 36.26 | 8 | O |
| ATOM | 1763 | N | LYS | A | 224 | 5.724 | 14.187 | 20.764 | 1.00 | 26.21 | 7 | N |
| ATOM | 1764 | CA | LYS | A | 224 | 4.900 | 13.015 | 20.459 | 1.00 | 26.55 | 6 | C |
| ATOM | 1765 | C | LYS | A | 224 | 5.393 | 11.824 | 21.274 | 1.00 | 26.18 | 6 | C |
| ATOM | 1766 | O | LYS | A | 224 | 4.595 | 11.041 | 21.791 | 1.00 | 26.77 | 8 | O |
| ATOM | 1767 | CB | LYS | A | 224 | 4.855 | 12.753 | 18.957 | 1.00 | 28.51 | 6 | C |
| ATOM | 1768 | CG | LYS | A | 224 | 4.221 | 13.855 | 18.126 | 1.00 | 30.72 | 6 | C |
| ATOM | 1769 | CD | LYS | A | 224 | 4.110 | 13.495 | 16.653 | 1.00 | 32.20 | 6 | C |
| ATOM | 1770 | CE | LYS | A | 224 | 3.616 | 14.673 | 15.822 | 1.00 | 33.79 | 6 | C |
| ATOM | 1771 | NZ | LYS | A | 224 | 2.575 | 14.227 | 14.858 | 1.00 | 35.71 | 7 | N |
| ATOM | 1772 | N | ILE | A | 225 | 6.708 | 11.670 | 21.440 | 1.00 | 24.40 | 7 | N |
| ATOM | 1773 | CA | ILE | A | 225 | 7.285 | 10.589 | 22.233 | 1.00 | 23.71 | 6 | C |
| ATOM | 1774 | C | ILE | A | 225 | 7.118 | 10.817 | 23.728 | 1.00 | 24.26 | 6 | C |
| ATOM | 1775 | O | ILE | A | 225 | 6.660 | 9.960 | 24.487 | 1.00 | 23.93 | 8 | O |
| ATOM | 1776 | CB | ILE | A | 225 | 8.795 | 10.472 | 21.896 | 1.00 | 23.02 | 6 | C |
| ATOM | 1777 | CG1 | ILE | A | 225 | 8.920 | 10.275 | 20.380 | 1.00 | 22.03 | 6 | C |
| ATOM | 1778 | CG2 | ILE | A | 225 | 9.442 | 9.341 | 22.672 | 1.00 | 21.86 | 6 | C |
| ATOM | 1779 | CD1 | ILE | A | 225 | 10.324 | 10.373 | 19.822 | 1.00 | 22.62 | 6 | C |
| ATOM | 1780 | N | ALA | A | 226 | 7.468 | 12.014 | 24.201 | 1.00 | 24.58 | 7 | N |
| ATOM | 1781 | CA | ALA | A | 226 | 7.375 | 12.383 | 25.607 | 1.00 | 25.90 | 6 | C |
| ATOM | 1782 | C | ALA | A | 226 | 6.004 | 12.146 | 26.213 | 1.00 | 25.99 | 6 | C |
| ATOM | 1783 | O | ALA | A | 226 | 5.838 | 11.618 | 27.327 | 1.00 | 25.98 | 8 | O |
| ATOM | 1784 | CB | ALA | A | 226 | 7.715 | 13.872 | 25.765 | 1.00 | 25.74 | 6 | C |
| ATOM | 1785 | N | ARG | A | 227 | 4.959 | 12.515 | 25.474 | 1.00 | 26.58 | 7 | N |
| ATOM | 1786 | CA | ARG | A | 227 | 3.596 | 12.321 | 25.981 | 1.00 | 28.10 | 6 | C |
| ATOM | 1787 | C | ARG | A | 227 | 3.224 | 10.868 | 26.163 | 1.00 | 27.85 | 6 | C |
| ATOM | 1788 | O | ARG | A | 227 | 2.332 | 10.556 | 26.959 | 1.00 | 28.64 | 8 | O |
| ATOM | 1789 | CB | ARG | A | 227 | 2.621 | 13.092 | 25.099 | 1.00 | 28.39 | 6 | C |
| ATOM | 1790 | CG | ARG | A | 227 | 2.948 | 14.584 | 25.165 | 1.00 | 30.15 | 6 | C |
| ATOM | 1791 | CD | ARG | A | 227 | 1.873 | 15.343 | 24.394 | 1.00 | 31.45 | 6 | C |
| ATOM | 1792 | NE | ARG | A | 227 | 0.565 | 15.082 | 25.010 | 1.00 | 31.98 | 7 | N |
| ATOM | 1793 | CZ | ARG | A | 227 | 0.158 | 15.732 | 26.096 | 1.00 | 32.26 | 6 | C |
| ATOM | 1794 | NH1 | ARG | A | 227 | 0.942 | 16.648 | 26.653 | 1.00 | 32.71 | 7 | N |
| ATOM | 1795 | NH2 | ARG | A | 227 | −1.036 | 15.444 | 26.597 | 1.00 | 33.16 | 7 | N |
| ATOM | 1796 | N | LEU | A | 228 | 3.899 | 9.941 | 25.489 | 1.00 | 27.85 | 7 | N |
| ATOM | 1797 | CA | LEU | A | 228 | 3.608 | 8.523 | 25.657 | 1.00 | 27.15 | 6 | C |
| ATOM | 1798 | C | LEU | A | 228 | 4.104 | 8.030 | 27.004 | 1.00 | 27.08 | 6 | C |
| ATOM | 1799 | O | LEU | A | 228 | 3.607 | 6.993 | 27.459 | 1.00 | 28.40 | 8 | O |
| ATOM | 1800 | CB | LEU | A | 228 | 4.152 | 7.735 | 24.466 | 1.00 | 27.46 | 6 | C |
| ATOM | 1801 | CG | LEU | A | 228 | 3.307 | 7.985 | 23.198 | 1.00 | 26.79 | 6 | C |
| ATOM | 1802 | CD1 | LEU | A | 228 | 4.124 | 7.734 | 21.948 | 1.00 | 27.68 | 6 | C |
| ATOM | 1803 | CD2 | LEU | A | 228 | 2.054 | 7.131 | 23.243 | 1.00 | 26.06 | 6 | C |
| ATOM | 1804 | N | GLY | A | 229 | 5.032 | 8.721 | 27.664 | 1.00 | 26.34 | 7 | N |
| ATOM | 1805 | CA | GLY | A | 229 | 5.492 | 8.316 | 28.977 | 1.00 | 25.42 | 6 | C |
| ATOM | 1806 | C | GLY | A | 229 | 6.968 | 7.972 | 29.068 | 1.00 | 25.21 | 6 | C |
| ATOM | 1807 | O | GLY | A | 229 | 7.454 | 7.608 | 30.139 | 1.00 | 25.16 | 8 | O |
| ATOM | 1808 | N | TYR | A | 230 | 7.684 | 8.074 | 27.957 | 1.00 | 24.55 | 7 | N |
| ATOM | 1809 | CA | TYR | A | 230 | 9.100 | 7.765 | 27.925 | 1.00 | 24.03 | 6 | C |
| ATOM | 1810 | C | TYR | A | 230 | 9.976 | 8.921 | 28.400 | 1.00 | 24.53 | 6 | C |
| ATOM | 1811 | O | TYR | A | 230 | 9.639 | 10.083 | 28.236 | 1.00 | 23.72 | 8 | O |
| ATOM | 1812 | CB | TYR | A | 230 | 9.535 | 7.443 | 26.490 | 1.00 | 23.08 | 6 | C |
| ATOM | 1813 | CG | TYR | A | 230 | 8.782 | 6.307 | 25.841 | 1.00 | 22.19 | 6 | C |
| ATOM | 1814 | CD1 | TYR | A | 230 | 9.031 | 4.993 | 26.199 | 1.00 | 21.74 | 6 | C |
| ATOM | 1815 | CD2 | TYR | A | 230 | 7.821 | 6.548 | 24.870 | 1.00 | 22.54 | 6 | C |
| ATOM | 1816 | CE1 | TYR | A | 230 | 8.351 | 3.939 | 25.610 | 1.00 | 22.29 | 6 | C |
| ATOM | 1817 | CE2 | TYR | A | 230 | 7.129 | 5.512 | 24.269 | 1.00 | 21.94 | 6 | C |
| ATOM | 1818 | CZ | TYR | A | 230 | 7.399 | 4.212 | 24.645 | 1.00 | 22.28 | 6 | C |
| ATOM | 1819 | OH | TYR | A | 230 | 6.702 | 3.191 | 24.038 | 1.00 | 23.27 | 8 | O |
| ATOM | 1820 | N | ARG | A | 231 | 11.131 | 8.570 | 28.974 | 1.00 | 25.30 | 7 | N |
| ATOM | 1821 | CA | ARG | A | 231 | 12.093 | 9.584 | 29.398 | 1.00 | 25.87 | 6 | C |
| ATOM | 1822 | C | ARG | A | 231 | 12.892 | 9.916 | 28.130 | 1.00 | 25.03 | 6 | C |
| ATOM | 1823 | O | ARG | A | 231 | 13.687 | 9.077 | 27.697 | 1.00 | 23.97 | 8 | O |
| ATOM | 1824 | CB | ARG | A | 231 | 13.050 | 9.086 | 30.472 | 1.00 | 28.26 | 6 | C |
| ATOM | 1825 | CG | ARG | A | 231 | 12.355 | 8.833 | 31.800 | 1.00 | 33.12 | 6 | C |
| ATOM | 1826 | CD | ARG | A | 231 | 13.307 | 8.314 | 32.861 | 1.00 | 35.40 | 6 | C |
| ATOM | 1827 | NE | ARG | A | 231 | 14.391 | 9.251 | 33.145 | 1.00 | 37.82 | 7 | N |
| ATOM | 1828 | CZ | ARG | A | 231 | 15.365 | 8.969 | 34.000 | 1.00 | 39.64 | 6 | C |
| ATOM | 1829 | NH1 | ARG | A | 231 | 15.409 | 7.817 | 34.651 | 1.00 | 39.95 | 7 | N |
| ATOM | 1830 | NH2 | ARG | A | 231 | 16.330 | 9.858 | 34.225 | 1.00 | 41.55 | 7 | N |
| ATOM | 1831 | N | VAL | A | 232 | 12.651 | 11.094 | 27.556 | 1.00 | 23.31 | 7 | N |
| ATOM | 1832 | CA | VAL | A | 232 | 13.362 | 11.420 | 26.317 | 1.00 | 23.40 | 6 | C |
| ATOM | 1833 | C | VAL | A | 232 | 14.688 | 12.125 | 26.529 | 1.00 | 23.66 | 6 | C |
| ATOM | 1834 | O | VAL | A | 232 | 14.821 | 13.082 | 27.290 | 1.00 | 23.96 | 8 | O |
| ATOM | 1835 | CB | VAL | A | 232 | 12.434 | 12.208 | 25.368 | 1.00 | 23.57 | 6 | C |
| ATOM | 1836 | CG1 | VAL | A | 232 | 13.100 | 12.478 | 24.023 | 1.00 | 23.45 | 6 | C |
| ATOM | 1837 | CG2 | VAL | A | 232 | 11.125 | 11.448 | 25.179 | 1.00 | 23.12 | 6 | C |
| ATOM | 1838 | N | ILE | A | 233 | 15.704 | 11.612 | 25.836 | 1.00 | 21.72 | 7 | N |
| ATOM | 1839 | CA | ILE | A | 233 | 17.055 | 12.151 | 25.890 | 1.00 | 22.61 | 6 | C |
| ATOM | 1840 | C | ILE | A | 233 | 17.415 | 12.657 | 24.490 | 1.00 | 21.63 | 6 | C |
| ATOM | 1841 | O | ILE | A | 233 | 17.354 | 11.872 | 23.540 | 1.00 | 22.10 | 8 | O |
| ATOM | 1842 | CB | ILE | A | 233 | 18.088 | 11.093 | 26.327 | 1.00 | 22.07 | 6 | C |
| ATOM | 1843 | CG1 | ILE | A | 233 | 17.693 | 10.469 | 27.667 | 1.00 | 23.64 | 6 | C |
| ATOM | 1844 | CG2 | ILE | A | 233 | 19.481 | 11.712 | 26.380 | 1.00 | 22.58 | 6 | C |
| ATOM | 1845 | CD1 | ILE | A | 233 | 18.523 | 9.259 | 28.056 | 1.00 | 22.98 | 6 | C |
| ATOM | 1846 | N | GLU | A | 234 | 17.771 | 13.932 | 24.347 | 1.00 | 20.67 | 7 | N |
| ATOM | 1847 | CA | GLU | A | 234 | 18.117 | 14.455 | 23.029 | 1.00 | 20.08 | 6 | C |
| ATOM | 1848 | C | GLU | A | 234 | 19.629 | 14.545 | 22.868 | 1.00 | 19.94 | 6 | C |
| ATOM | 1849 | O | GLU | A | 234 | 20.310 | 15.075 | 23.756 | 1.00 | 17.71 | 8 | O |
| ATOM | 1850 | CB | GLU | A | 234 | 17.534 | 15.862 | 22.804 | 1.00 | 21.77 | 6 | C |
| ATOM | 1851 | CG | GLU | A | 234 | 16.017 | 15.886 | 22.718 | 1.00 | 24.10 | 6 | C |
| ATOM | 1852 | CD | GLU | A | 234 | 15.458 | 17.306 | 22.699 | 1.00 | 26.00 | 6 | C |
| ATOM | 1853 | OE1 | GLU | A | 234 | 15.131 | 17.851 | 21.629 | 1.00 | 27.63 | 8 | O |
| ATOM | 1854 | OE2 | GLU | A | 234 | 15.342 | 17.889 | 23.795 | 1.00 | 26.36 | 8 | O |
| ATOM | 1855 | N | VAL | A | 235 | 20.137 | 14.016 | 21.767 | 1.00 | 18.16 | 7 | N |
| ATOM | 1856 | CA | VAL | A | 235 | 21.559 | 14.027 | 21.464 | 1.00 | 18.73 | 6 | C |
| ATOM | 1857 | C | VAL | A | 235 | 21.766 | 14.590 | 20.059 | 1.00 | 19.41 | 6 | C |
| ATOM | 1858 | O | VAL | A | 235 | 21.174 | 14.066 | 19.111 | 1.00 | 18.00 | 8 | O |
| ATOM | 1859 | CB | VAL | A | 235 | 22.209 | 12.632 | 21.529 | 1.00 | 17.92 | 6 | C |
| ATOM | 1860 | CG1 | VAL | A | 235 | 23.665 | 12.668 | 21.071 | 1.00 | 18.76 | 6 | C |
| ATOM | 1861 | CG2 | VAL | A | 235 | 22.153 | 12.052 | 22.935 | 1.00 | 18.57 | 6 | C |
| ATOM | 1862 | N | ASP | A | 236 | 22.602 | 15.622 | 19.913 | 1.00 | 19.80 | 7 | N |
| ATOM | 1863 | CA | ASP | A | 236 | 22.796 | 16.157 | 18.547 | 1.00 | 19.66 | 6 | C |
| ATOM | 1864 | C | ASP | A | 236 | 23.608 | 15.166 | 17.731 | 1.00 | 19.50 | 6 | C |
| ATOM | 1865 | O | ASP | A | 236 | 24.700 | 14.781 | 18.165 | 1.00 | 17.44 | 8 | O |
| ATOM | 1866 | CB | ASP | A | 236 | 23.469 | 17.524 | 18.609 | 1.00 | 20.81 | 6 | C |
| ATOM | 1867 | CG | ASP | A | 236 | 23.594 | 18.256 | 17.289 | 1.00 | 21.85 | 6 | C |
| ATOM | 1868 | OD1 | ASP | A | 236 | 23.060 | 17.829 | 16.243 | 1.00 | 19.75 | 8 | O |
| ATOM | 1869 | OD2 | ASP | A | 236 | 24.262 | 19.323 | 17.297 | 1.00 | 22.10 | 8 | O |
| ATOM | 1870 | N | THR | A | 237 | 23.106 | 14.698 | 16.591 | 1.00 | 18.51 | 7 | N |
| ATOM | 1871 | CA | THR | A | 237 | 23.826 | 13.749 | 15.755 | 1.00 | 18.19 | 6 | C |
| ATOM | 1872 | C | THR | A | 237 | 23.990 | 14.304 | 14.338 | 1.00 | 18.70 | 6 | C |
| ATOM | 1873 | O | THR | A | 237 | 24.121 | 13.572 | 13.353 | 1.00 | 20.04 | 8 | O |
| ATOM | 1874 | CB | THR | A | 237 | 23.142 | 12.371 | 15.649 | 1.00 | 19.37 | 6 | C |
| ATOM | 1875 | OG1 | THR | A | 237 | 21.820 | 12.501 | 15.059 | 1.00 | 19.13 | 8 | O |
| ATOM | 1876 | CG2 | THR | A | 237 | 22.981 | 11.673 | 16.994 | 1.00 | 18.54 | 6 | C |
| ATOM | 1877 | N | SER | A | 238 | 24.034 | 15.623 | 14.197 | 1.00 | 18.41 | 7 | N |
| ATOM | 1878 | CA | SER | A | 238 | 24.144 | 16.283 | 12.908 | 1.00 | 18.50 | 6 | C |
| ATOM | 1879 | C | SER | A | 238 | 25.284 | 15.803 | 12.037 | 1.00 | 18.04 | 6 | C |
| ATOM | 1880 | O | SER | A | 238 | 25.122 | 15.749 | 10.804 | 1.00 | 16.58 | 8 | O |
| ATOM | 1881 | CB | SER | A | 238 | 24.244 | 17.804 | 13.103 | 1.00 | 19.29 | 6 | C |
| ATOM | 1882 | OG | SER | A | 238 | 25.573 | 18.173 | 13.435 | 1.00 | 20.90 | 8 | O |
| ATOM | 1883 | N | GLU | A | 239 | 26.421 | 15.427 | 12.596 | 1.00 | 16.91 | 7 | N |
| ATOM | 1884 | CA | GLU | A | 239 | 27.553 | 14.960 | 11.786 | 1.00 | 17.30 | 6 | C |
| ATOM | 1885 | C | GLU | A | 239 | 27.290 | 13.574 | 11.217 | 1.00 | 17.93 | 6 | C |
| ATOM | 1886 | O | GLU | A | 239 | 27.825 | 13.247 | 10.153 | 1.00 | 17.75 | 8 | O |
| ATOM | 1887 | CB | GLU | A | 239 | 28.848 | 15.042 | 12.596 | 1.00 | 18.76 | 6 | C |
| ATOM | 1888 | CG | GLU | A | 239 | 29.199 | 16.464 | 13.013 | 1.00 | 19.04 | 6 | C |
| ATOM | 1889 | CD | GLU | A | 239 | 29.011 | 17.507 | 11.926 | 1.00 | 21.16 | 6 | C |
| ATOM | 1890 | OE1 | GLU | A | 239 | 29.815 | 17.652 | 10.980 | 1.00 | 22.26 | 8 | O |
| ATOM | 1891 | OE2 | GLU | A | 239 | 27.984 | 18.210 | 12.052 | 1.00 | 22.83 | 8 | O |
| ATOM | 1892 | N | TYR | A | 240 | 26.458 | 12.789 | 11.897 | 1.00 | 17.97 | 7 | N |
| ATOM | 1893 | CA | TYR | A | 240 | 26.133 | 11.449 | 11.367 | 1.00 | 18.50 | 6 | C |
| ATOM | 1894 | C | TYR | A | 240 | 25.018 | 11.564 | 10.344 | 1.00 | 17.44 | 6 | C |
| ATOM | 1895 | O | TYR | A | 240 | 24.976 | 10.772 | 9.391 | 1.00 | 15.89 | 8 | O |
| ATOM | 1896 | CB | TYR | A | 240 | 25.863 | 10.463 | 12.505 | 1.00 | 18.57 | 6 | C |
| ATOM | 1897 | CG | TYR | A | 240 | 27.109 | 10.420 | 13.386 | 1.00 | 17.89 | 6 | C |
| ATOM | 1898 | CD1 | TYR | A | 240 | 27.177 | 11.138 | 14.566 | 1.00 | 18.23 | 6 | C |
| ATOM | 1899 | CD2 | TYR | A | 240 | 28.200 | 9.662 | 12.993 | 1.00 | 18.13 | 6 | C |
| ATOM | 1900 | CE1 | TYR | A | 240 | 28.321 | 11.097 | 15.354 | 1.00 | 18.33 | 6 | C |
| ATOM | 1901 | CE2 | TYR | A | 240 | 29.345 | 9.608 | 13.764 | 1.00 | 18.20 | 6 | C |
| ATOM | 1902 | CZ | TYR | A | 240 | 29.394 | 10.330 | 14.942 | 1.00 | 18.65 | 6 | C |
| ATOM | 1903 | OH | TYR | A | 240 | 30.530 | 10.292 | 15.723 | 1.00 | 16.42 | 8 | O |
| ATOM | 1904 | N | ARG | A | 241 | 24.116 | 12.532 | 10.490 | 1.00 | 18.43 | 7 | N |
| ATOM | 1905 | CA | ARG | A | 241 | 23.041 | 12.783 | 9.530 | 1.00 | 19.19 | 6 | C |
| ATOM | 1906 | C | ARG | A | 241 | 23.609 | 13.029 | 8.145 | 1.00 | 19.02 | 6 | C |
| ATOM | 1907 | O | ARG | A | 241 | 23.073 | 12.609 | 7.091 | 1.00 | 18.91 | 8 | O |
| ATOM | 1908 | CB | ARG | A | 241 | 22.165 | 13.968 | 9.988 | 1.00 | 20.02 | 6 | C |
| ATOM | 1909 | CG | ARG | A | 241 | 21.087 | 14.325 | 8.956 | 1.00 | 22.75 | 6 | C |
| ATOM | 1910 | CD | ARG | A | 241 | 20.057 | 15.275 | 9.541 | 1.00 | 25.11 | 6 | C |
| ATOM | 1911 | NE | ARG | A | 241 | 19.186 | 15.852 | 8.522 | 1.00 | 27.08 | 7 | N |
| ATOM | 1912 | CZ | ARG | A | 241 | 19.279 | 17.033 | 7.933 | 1.00 | 29.17 | 6 | C |
| ATOM | 1913 | NH1 | ARG | A | 241 | 20.249 | 17.898 | 8.224 | 1.00 | 29.86 | 7 | N |
| ATOM | 1914 | NH2 | ARG | A | 241 | 18.371 | 17.381 | 7.014 | 1.00 | 27.92 | 7 | N |
| ATOM | 1915 | N | LYS | A | 242 | 24.774 | 13.691 | 8.070 | 1.00 | 18.20 | 7 | N |
| ATOM | 1916 | CA | LYS | A | 242 | 25.480 | 13.972 | 6.847 | 1.00 | 18.03 | 6 | C |
| ATOM | 1917 | C | LYS | A | 242 | 25.857 | 12.714 | 6.069 | 1.00 | 17.62 | 6 | C |
| ATOM | 1918 | O | LYS | A | 242 | 25.978 | 12.777 | 4.843 | 1.00 | 17.63 | 8 | O |
| ATOM | 1919 | CB | LYS | A | 242 | 26.773 | 14.769 | 7.080 | 1.00 | 17.22 | 6 | C |
| ATOM | 1920 | CG | LYS | A | 242 | 26.509 | 16.211 | 7.487 | 1.00 | 17.00 | 6 | C |
| ATOM | 1921 | CD | LYS | A | 242 | 27.802 | 16.864 | 7.984 | 1.00 | 18.03 | 6 | C |
| ATOM | 1922 | CE | LYS | A | 242 | 27.538 | 18.306 | 8.410 | 1.00 | 18.91 | 6 | C |
| ATOM | 1923 | NZ | LYS | A | 242 | 28.841 | 18.933 | 8.805 | 1.00 | 20.60 | 7 | N |
| ATOM | 1924 | N | ILE | A | 243 | 26.077 | 11.608 | 6.780 | 1.00 | 17.54 | 7 | N |
| ATOM | 1925 | CA | ILE | A | 243 | 26.426 | 10.352 | 6.142 | 1.00 | 17.39 | 6 | C |
| ATOM | 1926 | C | ILE | A | 243 | 25.327 | 9.323 | 6.402 | 1.00 | 16.97 | 6 | C |
| ATOM | 1927 | O | ILE | A | 243 | 25.583 | 8.140 | 6.634 | 1.00 | 16.25 | 8 | O |
| ATOM | 1928 | CB | ILE | A | 243 | 27.846 | 9.837 | 6.442 | 1.00 | 17.98 | 6 | C |
| ATOM | 1929 | CG1 | ILE | A | 243 | 28.115 | 9.747 | 7.943 | 1.00 | 18.02 | 6 | C |
| ATOM | 1930 | CG2 | ILE | A | 243 | 28.873 | 10.743 | 5.752 | 1.00 | 17.31 | 6 | C |
| ATOM | 1931 | CD1 | ILE | A | 243 | 29.518 | 9.288 | 8.323 | 1.00 | 19.90 | 6 | C |
| ATOM | 1932 | N | ASP | A | 244 | 24.076 | 9.779 | 6.355 | 1.00 | 15.92 | 7 | N |
| ATOM | 1933 | CA | ASP | A | 244 | 22.878 | 8.966 | 6.479 | 1.00 | 16.66 | 6 | C |
| ATOM | 1934 | C | ASP | A | 244 | 22.770 | 8.010 | 7.653 | 1.00 | 16.51 | 6 | C |
| ATOM | 1935 | O | ASP | A | 244 | 22.238 | 6.905 | 7.518 | 1.00 | 16.01 | 8 | O |
| ATOM | 1936 | CB | ASP | A | 244 | 22.735 | 8.125 | 5.185 | 1.00 | 19.19 | 6 | C |
| ATOM | 1937 | CG | ASP | A | 244 | 22.664 | 8.909 | 3.893 | 1.00 | 21.53 | 6 | C |
| ATOM | 1938 | OD1 | ASP | A | 244 | 22.198 | 10.070 | 3.935 | 1.00 | 22.46 | 8 | O |
| ATOM | 1939 | OD2 | ASP | A | 244 | 23.079 | 8.377 | 2.825 | 1.00 | 19.93 | 8 | O |
| ATOM | 1940 | N | GLY | A | 245 | 23.216 | 8.429 | 8.834 | 1.00 | 15.82 | 7 | N |
| ATOM | 1941 | CA | GLY | A | 245 | 23.132 | 7.582 | 10.022 | 1.00 | 17.08 | 6 | C |
| ATOM | 1942 | C | GLY | A | 245 | 22.547 | 8.366 | 11.200 | 1.00 | 17.92 | 6 | C |
| ATOM | 1943 | O | GLY | A | 245 | 22.580 | 9.599 | 11.213 | 1.00 | 19.55 | 8 | O |
| ATOM | 1944 | N | GLY | A | 246 | 22.052 | 7.646 | 12.190 | 1.00 | 18.13 | 7 | N |
| ATOM | 1945 | CA | GLY | A | 246 | 21.463 | 8.233 | 13.381 | 1.00 | 18.79 | 6 | C |
| ATOM | 1946 | C | GLY | A | 246 | 21.759 | 7.412 | 14.639 | 1.00 | 18.51 | 6 | C |
| ATOM | 1947 | O | GLY | A | 246 | 22.697 | 6.625 | 14.696 | 1.00 | 17.02 | 8 | O |
| ATOM | 1948 | N | VAL | A | 247 | 20.900 | 7.588 | 15.643 | 1.00 | 18.26 | 7 | N |
| ATOM | 1949 | CA | VAL | A | 247 | 21.028 | 6.901 | 16.910 | 1.00 | 17.02 | 6 | C |
| ATOM | 1950 | C | VAL | A | 247 | 20.815 | 5.397 | 16.811 | 1.00 | 16.78 | 6 | C |
| ATOM | 1951 | O | VAL | A | 247 | 21.485 | 4.707 | 17.571 | 1.00 | 14.90 | 8 | O |
| ATOM | 1952 | CB | VAL | A | 247 | 20.151 | 7.505 | 18.013 | 1.00 | 17.66 | 6 | C |
| ATOM | 1953 | CG1 | VAL | A | 247 | 20.571 | 8.956 | 18.289 | 1.00 | 17.87 | 6 | C |
| ATOM | 1954 | CG2 | VAL | A | 247 | 18.659 | 7.481 | 17.712 | 1.00 | 17.27 | 6 | C |
| ATOM | 1955 | N | SER | A | 248 | 19.977 | 4.842 | 15.928 | 1.00 | 15.01 | 7 | N |
| ATOM | 1956 | CA | SER | A | 248 | 19.850 | 3.385 | 15.903 | 1.00 | 16.22 | 6 | C |
| ATOM | 1957 | C | SER | A | 248 | 21.118 | 2.779 | 15.312 | 1.00 | 15.38 | 6 | C |
| ATOM | 1958 | O | SER | A | 248 | 21.620 | 1.732 | 15.685 | 1.00 | 16.72 | 8 | O |
| ATOM | 1959 | CB | SER | A | 248 | 18.649 | 2.902 | 15.080 | 1.00 | 16.96 | 6 | C |
| ATOM | 1960 | OG | SER | A | 248 | 18.987 | 2.881 | 13.709 | 1.00 | 18.74 | 8 | O |
| ATOM | 1961 | N | SER | A | 249 | 21.689 | 3.474 | 14.348 | 1.00 | 14.73 | 7 | N |
| ATOM | 1962 | CA | SER | A | 249 | 22.862 | 3.102 | 13.602 | 1.00 | 15.13 | 6 | C |
| ATOM | 1963 | C | SER | A | 249 | 24.132 | 2.906 | 14.408 | 1.00 | 15.57 | 6 | C |
| ATOM | 1964 | O | SER | A | 249 | 24.965 | 2.095 | 14.023 | 1.00 | 14.24 | 8 | O |
| ATOM | 1965 | CB | SER | A | 249 | 23.194 | 4.254 | 12.612 | 1.00 | 13.25 | 6 | C |
| ATOM | 1966 | OG | SER | A | 249 | 21.956 | 4.758 | 12.111 | 1.00 | 15.85 | 8 | O |
| ATOM | 1967 | N | MET | A | 250 | 24.273 | 3.693 | 15.460 | 1.00 | 15.07 | 7 | N |
| ATOM | 1968 | CA | MET | A | 250 | 25.476 | 3.728 | 16.266 | 1.00 | 15.73 | 6 | C |
| ATOM | 1969 | C | MET | A | 250 | 25.609 | 2.735 | 17.392 | 1.00 | 15.38 | 6 | C |
| ATOM | 1970 | O | MET | A | 250 | 26.597 | 2.812 | 18.153 | 1.00 | 14.65 | 8 | O |
| ATOM | 1971 | CB | MET | A | 250 | 25.652 | 5.201 | 16.711 | 1.00 | 15.84 | 6 | C |
| ATOM | 1972 | CG | MET | A | 250 | 26.100 | 5.999 | 15.474 | 1.00 | 17.65 | 6 | C |
| ATOM | 1973 | SD | MET | A | 250 | 26.603 | 7.690 | 15.880 | 1.00 | 20.47 | 16 | S |
| ATOM | 1974 | CE | MET | A | 250 | 25.035 | 8.280 | 16.521 | 1.00 | 17.66 | 6 | C |
| ATOM | 1975 | N | SER | A | 251 | 24.660 | 1.799 | 17.531 | 1.00 | 14.25 | 7 | N |
| ATOM | 1976 | CA | SER | A | 251 | 24.840 | 0.823 | 18.604 | 1.00 | 14.07 | 6 | C |
| ATOM | 1977 | C | SER | A | 251 | 24.130 | −0.486 | 18.289 | 1.00 | 15.68 | 6 | C |
| ATOM | 1978 | O | SER | A | 251 | 23.326 | −0.563 | 17.371 | 1.00 | 15.01 | 8 | O |
| ATOM | 1979 | CB | SER | A | 251 | 24.396 | 1.316 | 19.967 | 1.00 | 15.16 | 6 | C |
| ATOM | 1980 | OG | SER | A | 251 | 23.085 | 1.829 | 20.089 | 1.00 | 13.29 | 8 | O |
| ATOM | 1981 | N | LEU | A | 252 | 24.525 | −1.481 | 19.063 | 1.00 | 14.44 | 7 | N |
| ATOM | 1982 | CA | LEU | A | 252 | 23.952 | −2.819 | 19.087 | 1.00 | 16.14 | 6 | C |
| ATOM | 1983 | C | LEU | A | 252 | 23.320 | −2.862 | 20.492 | 1.00 | 16.24 | 6 | C |
| ATOM | 1984 | O | LEU | A | 252 | 23.999 | −2.484 | 21.461 | 1.00 | 16.21 | 8 | O |
| ATOM | 1985 | CB | LEU | A | 252 | 24.942 | −3.937 | 18.943 | 1.00 | 16.95 | 6 | C |
| ATOM | 1986 | CG | LEU | A | 252 | 25.367 | −4.646 | 17.676 | 1.00 | 19.53 | 6 | C |
| ATOM | 1987 | CD1 | LEU | A | 252 | 24.646 | −4.231 | 16.422 | 1.00 | 18.10 | 6 | C |
| ATOM | 1988 | CD2 | LEU | A | 252 | 26.874 | −4.715 | 17.496 | 1.00 | 18.43 | 6 | C |
| ATOM | 1989 | N | ARG | A | 253 | 22.064 | −3.213 | 20.654 | 1.00 | 16.41 | 7 | N |
| ATOM | 1990 | CA | ARG | A | 253 | 21.324 | −3.228 | 21.898 | 1.00 | 16.03 | 6 | C |
| ATOM | 1991 | C | ARG | A | 253 | 20.722 | −4.600 | 22.196 | 1.00 | 17.45 | 6 | C |
| ATOM | 1992 | O | ARG | A | 253 | 19.979 | −5.104 | 21.360 | 1.00 | 16.39 | 8 | O |
| ATOM | 1993 | CB | ARG | A | 253 | 20.185 | −2.192 | 21.764 | 1.00 | 15.49 | 6 | C |
| ATOM | 1994 | CG | ARG | A | 253 | 20.690 | −0.791 | 21.468 | 1.00 | 13.94 | 6 | C |
| ATOM | 1995 | CD | ARG | A | 253 | 19.624 | 0.188 | 21.007 | 1.00 | 13.72 | 6 | C |
| ATOM | 1996 | NE | ARG | A | 253 | 19.235 | −0.096 | 19.619 | 1.00 | 11.97 | 7 | N |
| ATOM | 1997 | CZ | ARG | A | 253 | 19.854 | 0.264 | 18.517 | 1.00 | 13.18 | 6 | C |
| ATOM | 1998 | NH1 | ARG | A | 253 | 19.299 | −0.105 | 17.343 | 1.00 | 14.32 | 7 | N |
| ATOM | 1999 | NH2 | ARG | A | 253 | 20.975 | 0.971 | 18.508 | 1.00 | 12.64 | 7 | N |
| ATOM | 2000 | N | PHE | A | 254 | 21.061 | −5.222 | 23.321 | 1.00 | 17.19 | 7 | N |
| ATOM | 2001 | CA | PHE | A | 254 | 20.571 | −6.548 | 23.650 | 1.00 | 17.99 | 6 | C |
| ATOM | 2002 | C | PHE | A | 254 | 20.654 | −6.944 | 25.125 | 1.00 | 19.16 | 6 | C |
| ATOM | 2003 | O | PHE | A | 254 | 20.966 | −6.025 | 25.938 | 1.00 | 17.69 | 8 | O |
| ATOM | 2004 | CB | PHE | A | 254 | 21.364 | −7.577 | 22.812 | 1.00 | 17.71 | 6 | C |
| ATOM | 2005 | CG | PHE | A | 254 | 22.853 | −7.600 | 23.036 | 1.00 | 18.58 | 6 | C |
| ATOM | 2006 | CD1 | PHE | A | 254 | 23.435 | −8.482 | 23.926 | 1.00 | 18.65 | 6 | C |
| ATOM | 2007 | CD2 | PHE | A | 254 | 23.676 | −6.727 | 22.348 | 1.00 | 17.76 | 6 | C |
| ATOM | 2008 | CE1 | PHE | A | 254 | 24.810 | −8.487 | 24.124 | 1.00 | 19.64 | 6 | C |
| ATOM | 2009 | CE2 | PHE | A | 254 | 25.047 | −6.726 | 22.528 | 1.00 | 20.27 | 6 | C |
| ATOM | 2010 | CZ | PHE | A | 254 | 25.624 | −7.618 | 23.426 | 1.00 | 19.50 | 6 | C |
| TER | | | | | | | | | | | | |
| ATOM | 9 | C1 | CIT | L | 1 | 26.383 | 3.969 | 4.880 | 1.00 | 18.49 | 6 | C |
| ATOM | 11 | O1 | CIT | L | 1 | 26.445 | 2.626 | 4.972 | 1.00 | 17.89 | 8 | O |
| ATOM | 10 | O2 | CIT | L | 1 | 26.091 | 4.486 | 3.823 | 1.00 | 18.49 | 8 | O |
| ATOM | 8 | C2 | CIT | L | 1 | 26.629 | 4.760 | 6.142 | 1.00 | 17.64 | 6 | C |
| ATOM | 12 | N2 | CIT | L | 1 | 27.235 | 6.047 | 5.751 | 1.00 | 17.56 | 7 | N |
| ATOM | 7 | C3 | CIT | L | 1 | 25.309 | 4.948 | 6.903 | 1.00 | 17.39 | 6 | C |
| ATOM | 6 | C4 | CIT | L | 1 | 24.879 | 3.598 | 7.515 | 1.00 | 17.03 | 6 | C |
| ATOM | 5 | C5 | CIT | L | 1 | 23.561 | 3.795 | 8.251 | 1.00 | 18.81 | 6 | C |
| ATOM | 4 | N6 | CIT | L | 1 | 23.116 | 2.545 | 8.863 | 1.00 | 18.52 | 7 | N |
| ATOM | 2 | C7 | CIT | L | 1 | 22.092 | 2.463 | 9.708 | 1.00 | 19.27 | 6 | C |
| ATOM | 3 | O7 | CIT | L | 1 | 21.402 | 3.409 | 10.078 | 1.00 | 18.88 | 8 | O |
| ATOM | 1 | N7 | CIT | L | 1 | 21.791 | 1.229 | 10.205 | 1.00 | 19.31 | 7 | N |
| TER | | | | | | | | | | | | |
| ATOM | 1 | O1 | HOH | Y | 1 | 29.062 | 3.474 | 17.537 | 1.00 | 15.96 | 8 | O |
| ATOM | 2 | O1 | HOH | Y | 2 | 20.323 | −3.682 | 18.259 | 1.00 | 20.75 | 8 | O |
| ATOM | 3 | O1 | HOH | Y | 3 | 21.445 | −2.124 | 16.238 | 1.00 | 17.04 | 8 | O |
| ATOM | 4 | O1 | HOH | Y | 4 | 33.556 | 1.521 | 6.459 | 1.00 | 16.87 | 8 | O |
| ATOM | 5 | O1 | HOH | Y | 5 | 18.912 | −4.075 | 13.865 | 1.00 | 15.74 | 8 | O |
| ATOM | 6 | O1 | HOH | Y | 6 | 16.718 | −1.733 | 19.066 | 1.00 | 16.13 | 8 | O |
| ATOM | 7 | O1 | HOH | Y | 7 | 32.333 | −6.481 | 25.915 | 1.00 | 60.29 | 8 | O |
| ATOM | 8 | O1 | HOH | Y | 8 | 10.979 | −5.361 | 20.462 | 1.00 | 19.40 | 8 | O |
| ATOM | 9 | O1 | HOH | Y | 9 | 13.195 | −2.535 | 17.977 | 1.00 | 19.61 | 8 | O |
| ATOM | 10 | O1 | HOH | Y | 10 | 12.359 | −1.563 | 20.681 | 1.00 | 18.63 | 8 | O |
| ATOM | 11 | O1 | HOH | Y | 11 | 32.603 | −3.419 | 13.213 | 1.00 | 20.01 | 8 | O |
| ATOM | 12 | O1 | HOH | Y | 12 | 21.509 | −4.522 | 14.646 | 1.00 | 13.91 | 8 | O |
| ATOM | 13 | O1 | HOH | Y | 13 | 15.075 | 3.567 | 14.148 | 1.00 | 18.95 | 8 | O |
| ATOM | 14 | O1 | HOH | Y | 14 | 13.956 | 1.524 | 19.398 | 1.00 | 17.14 | 8 | O |
| ATOM | 15 | O1 | HOH | Y | 15 | 21.003 | −6.055 | 16.919 | 1.00 | 19.00 | 8 | O |
| ATOM | 16 | O1 | HOH | Y | 16 | 32.167 | 8.294 | 32.298 | 1.00 | 19.00 | 8 | O |
| ATOM | 17 | O1 | HOH | Y | 17 | 35.589 | −7.181 | 18.133 | 1.00 | 20.66 | 8 | O |
| ATOM | 18 | O1 | HOH | Y | 18 | 22.621 | 3.599 | 34.009 | 1.00 | 20.96 | 8 | O |
| ATOM | 19 | O1 | HOH | Y | 19 | 30.923 | −1.511 | 12.305 | 1.00 | 20.05 | 8 | O |
| ATOM | 20 | O1 | HOH | Y | 20 | 35.724 | 3.555 | 13.449 | 1.00 | 20.95 | 8 | O |
| ATOM | 21 | O1 | HOH | Y | 21 | 12.062 | −7.713 | 21.688 | 1.00 | 23.83 | 8 | O |
| ATOM | 22 | O1 | HOH | Y | 22 | 15.011 | −22.594 | 9.691 | 1.00 | 21.01 | 8 | O |
| ATOM | 23 | O1 | HOH | Y | 23 | 2.643 | −7.183 | 10.821 | 1.00 | 26.23 | 8 | O |
| ATOM | 24 | O1 | HOH | Y | 24 | 15.066 | 1.849 | 16.396 | 1.00 | 19.85 | 8 | O |
| ATOM | 25 | O1 | HOH | Y | 25 | 15.126 | −3.402 | 16.116 | 1.00 | 19.57 | 8 | O |
| ATOM | 26 | O1 | HOH | Y | 26 | 32.656 | 2.915 | 3.918 | 1.00 | 19.80 | 8 | O |
| ATOM | 27 | O1 | HOH | Y | 27 | 28.202 | −4.029 | 26.810 | 1.00 | 20.30 | 8 | O |
| ATOM | 28 | O1 | HOH | Y | 28 | 16.425 | 8.911 | 9.683 | 1.00 | 20.68 | 8 | O |
| ATOM | 29 | O1 | HOH | Y | 29 | 13.191 | −0.276 | 16.179 | 1.00 | 21.08 | 8 | O |
| ATOM | 30 | O1 | HOH | Y | 30 | 24.468 | 13.242 | 26.524 | 1.00 | 23.31 | 8 | O |
| ATOM | 31 | O1 | HOH | Y | 31 | 13.588 | −18.049 | 4.960 | 1.00 | 20.13 | 8 | O |
| ATOM | 32 | O1 | HOH | Y | 32 | 35.490 | −6.520 | 22.597 | 1.00 | 25.94 | 8 | O |
| ATOM | 33 | O1 | HOH | Y | 33 | 24.980 | 20.832 | 15.135 | 1.00 | 23.73 | 8 | O |
| ATOM | 34 | O1 | HOH | Y | 34 | 17.830 | −4.296 | 19.108 | 1.00 | 21.03 | 8 | O |
| ATOM | 35 | O1 | HOH | Y | 35 | 19.902 | 10.374 | 15.090 | 1.00 | 21.32 | 8 | O |
| ATOM | 36 | O1 | HOH | Y | 36 | 14.598 | 16.681 | 19.217 | 1.00 | 22.22 | 8 | O |
| ATOM | 37 | O1 | HOH | Y | 37 | 38.054 | 3.952 | 21.350 | 1.00 | 21.27 | 8 | O |
| ATOM | 38 | O1 | HOH | Y | 38 | 5.734 | −7.825 | 7.432 | 1.00 | 20.54 | 8 | O |
| ATOM | 39 | O1 | HOH | Y | 39 | 18.556 | −11.765 | −3.800 | 1.00 | 22.03 | 8 | O |
| ATOM | 40 | O1 | HOH | Y | 40 | 20.336 | 16.185 | 12.754 | 1.00 | 26.13 | 8 | O |
| ATOM | 41 | O1 | HOH | Y | 41 | 17.638 | −6.870 | 17.851 | 1.00 | 27.88 | 8 | O |
| ATOM | 42 | O1 | HOH | Y | 42 | 20.695 | 16.322 | 15.604 | 1.00 | 21.48 | 8 | O |
| ATOM | 43 | O1 | HOH | Y | 43 | 10.008 | 6.471 | 2.603 | 1.00 | 26.59 | 8 | O |
| ATOM | 44 | O1 | HOH | Y | 44 | 27.263 | 1.931 | 31.447 | 1.00 | 22.23 | 8 | O |
| ATOM | 45 | O1 | HOH | Y | 45 | 9.253 | 21.264 | 13.505 | 1.00 | 28.25 | 8 | O |

TABLE IV
Functionally important residues in PaDDAH and the their
equivalents in other superfamily members.

| PaDDAW | DDAHI | DDAHII | AGAT | IPAT | DEIM | |
|---|---|---|---|---|---|---|
| 60 | D* | L | Y | H | N* | |
| 63 | F/Y | L | M | C | F* | |
| 65 | E | G | R | R | R* | |
| 66 | D | D | D | D | D/E | Not same |
| | | | | | | contacts |
| | | | | | | in PA, AT |
| 85 | R | R | R | R | R* | paired with |
| | | | | | | 66 |
| 88 | E* | E | E | E | E* | |
| 114+ | D/E | D | D | D | E* | |
| 117 | D* | D | D | N | D* | |
| 132 | R* | W | V | S | R* | |
| 161 | L | S | M | T | M* | |
| 162+ | H* | H | H | H | H* | |
| 164 | K | R | D | D | D | |
| 165## | T/S | A | A | S | T | |
| 201## | AYSIM | A | L | I | FYMH | |
| 249+ | C* | C at+2 | C | C | C* | |

+catalytic
##contact residue in AT, but not in DDAH
*conserved in known members of family

1. Use of the structural coordinates obtainable by subjecting a crystal comprising a dimethylarginine dimethylaminohydrolase (DDAH) or a fragment thereof or an arginine deiminase (DI) or a fragment thereof to X-ray diffraction measurements and deducing the structural coordinates from the diffraction measurements, to identify, screen, characterise, design or modify a chemical entity.

2. Use according to claim 1, wherein the DDAH is a bacterial DDAH.

3. Use according to claim 2, wherein the bacterial DDAH is Pseudomonas aeruginosa DDAH (PaDDAH).

4. Use according to claim 1, wherein the DI is a Pseudonionas aeruginosa DI (PaDI).

5. Use according to any one of the preceding claims, wherein the DDAH or DI is an inactive mutant of a wild-type DDAH or DI.

6. Use according to claim 5, wherein the DDAH or DI has been rendered inactive by mutating an amino acid equivalent to E114, H162 or C249 of PaDDAH or PaDI to a different amino acid.

7. Use according to claim 6, wherein the different amino acid is alanine or a sterically similar amino acid.

8. Use according to any one of claims 5 to 7, wherein the crystal is a crystal of the DDAH or DI bound to a substrate or product.

9. Use of the structural coordinates set out in Table II to identify, screen, characterise, design or modify a chemical entity.

10. Use according to any one of the preceding claims, wherein the chemical entity is a modified DDAH or DI.

11. Use according to claim 10, wherein the modified DDAH or DI is inactive.

12. Use according to any one of claims 1 to 9, wherein the chemical entity binds to a DDAH or a DI.

13. Use according to claim 12, wherein the chemical entity is an inhibitor or an activator of a DDAH or a DI.

14. A chemical entity as defined in any one of the preceding claims.

15. A chemical entity as defined in claim 14 for use in a method for treatment of the human or animal body by therapy.

16. A chemical entity according to claim 15 for use in a method for treatment of a condition in which abnormal nitric oxide metabolism is implicated or for use in a method for treatment of a bacterial infection.

17. A method for identifying, screening, characterising or designing a chemical entity which is a modified DDAH or DI or binds to a DDAH or DI, which method comprises comparing a structural model of the DDAH or DI with a structural model for said chemical entity, and thereby determining whether said chemical entity is likely to be a modified DDAH or DI or bind to the DDAH or DI, wherein said structural model of the DDAH is derived from structural coordinates determined by subjecting to X-Ray diffraction measurements a crystal comprising a DDAH or DI as defined in any one of claims 1 to 8 or a fragment thereof.

18. A chemical entity identified by a method according to claim 17.

19. A chemical entity according to claim 18 for use in a method for treatment of the human or animal body by therapy.

20. A chemical entity according to claim 19 for use in a method for treatment of a condition in which abnormal nitric oxide metabolism is implicated or for use in a method for treatment of a bacterial infection.

21. A pharmaceutical composition comprising a chemical entity as defined in claim 14 or a chemical entity according to claim 18 and a pharmaceutically acceptable carrier or diluent.

22. A method for treating a host suffering from a condition in which abnormal nitric oxide metabolism is implicated or a bacterial infection, which method comprises administering to the host a therapeutically effective amount of a chemical entity as defined in claim 14 or a chemical entity according to claim 18.

23. A method for identifying the presence or absence of an asymmetrically methylated arginine derivative in a sample, which method comprises:
(a) contacting the sample with a DDAH as defined in any one of claims 5 to 7, a modified DDAH as defined in claim 10 or 11 or a modified DDAH identified by a method according to claim 17; and
(b) determining whether the DDAH binds to an asymmetrically methylated arginine derivative.

24. A crystal comprising a DDAH or a fragment thereof or a DI or a fragment thereof.

25. A crystal according to claim 24, wherein the DDAH is a DDAH as defined in any one of claims 1 to 3 or 5 to 8.

26. A crystal according to claim 24, wherein the DI is a DI as defined in any one of claims 1 or 4 to 8.

27. A method for preparing a crystal, which method comprises forming a crystal of a substance comprising a DDAH as defined in any one of claims 1 to 3 or 5 to 8.

28. A method for preparing a crystal, which method comprises forming a crystal of a substance comprising a DI as defined in any one of claims 1 or 4 to 8.

29. A DDAH or DI as defined in any one of claims 5 to 8.

30. A machine readable data storage medium comprising a data storage material encoded with machine readable data which when read by an appropriate machine is capable of displaying a three dimensional representation of a crystal as defined in claim 24 to 26.
